(19) United States
(12) Patent Application Publication
(10) Pub. No.: US 2023/0128499 A1
(43) Pub. Date: Apr. 27, 2023

(54) BISPECIFIC COMBINATION THERAPY FOR TREATING PROLIFERATIVE DISEASES AND AUTOIMMUNE DISEASES

(71) Applicants: Carl Uli BIALUCHA, Winchester, MA (US); Brian GRANDA, Cambridge, MA (US); Novartis AG, Basel (CH)

(72) Inventors: Carl Uli BIALUCHA, Winchester, MA (US); Brian GRANDA, Cambridge, MA (US)

(73) Assignee: Novartis AG, Basel (CH)

(21) Appl. No.: 17/907,300

(22) PCT Filed: Mar. 26, 2021
(86) PCT No.: PCT/US2021/024383

§ 371 Date: Sep. 26, 2022

Related U.S. Application Data
(60) Provisional application No. 63/111,852, filed on Nov. 10, 2020.
(60) Provisional application No. 63/000,693, filed on Mar. 27, 2020.

(51) Int. Cl. C07K 16/28; A61P 35/00; C07K 16/18

(52) U.S. Cl. CPC C07K 16/2806; C07K 16/2809; C07K 16/2824; A61P 35/00; C07K 16/2878; C07K 16/2803; C07K 16/18; C07K 16/2818; C07K 2317/31; C07K 2317/33; C07K 2317/76; C07K 2317/92; C07K 2317/52; C07K 2317/73; C07K 2317/622; C07K 2317/55

(57) ABSTRACT

The present disclosure provides methods of treating a subject having a proliferative disease or an autoimmune disorder with a combination of (a) a first multispecific binding molecule (MBM) that binds specifically to (i) human CD2 and (ii) a human tumor-associated antigen and/or a human tumor microenvironment antigen, and (b) a second multispecific binding molecule that binds specifically to (i) a component of a human T-cell receptor (TCR) complex or a secondary T-cell signaling molecule and (ii) a human tumor-associated antigen and/or human tumor microenvironment antigen. The disclosure further provides MBMs and combinations of MBMs that can be used in the methods of the disclosure.

### 1. CROSS-REFERENCE TO RELATED APPLICATIONS

This application claims the priority benefit of U.S. provisional application Nos. 63/000,693, filed Mar. 27, 2020, and 63/111,852, filed Nov. 10, 2020, the contents of both of which are incorporated herein in their entireties by reference thereto.

### 2. SEQUENCE LISTING

The instant application contains a Sequence Listing which has been submitted electronically in ASCII format and is hereby incorporated by reference in its entirety. Said ASCII copy, created on Mar. 22, 2021, is named NOV-009WO_SL.txt and is 774,367 bytes in size

### 3. FIELD OF INVENTION

This disclosure generally relates to combinations of multispecific binding molecules for treating proliferative diseases and autoimmune disorders, where a combination typically comprises (a) a first multispecific binding molecule (MBM) that binds specifically to (i) human CD2 and (ii) a human tumor-associated antigen and/or a human tumor microenvironment antigen, and (b) a second multispecific binding molecule that binds specifically to (i) a component of a human T-cell receptor (TCR) complex or a secondary T-cell signaling molecule and (ii) a human tumor-associated antigen and/or human tumor microenvironment antigen.

### 4. BACKGROUND

Redirected targeted T-cell lysis (RTCC) is an exciting mechanism for first line treatment and refractory settings. Antibodies and antibody fragments with their exquisite selectivity have been successfully engineered in a variety of formats to allow for the dual specificities required to cross-link T-cells to a single receptor on the target cell.

There is a need for improved RTCC approaches.

### 5. SUMMARY

The present disclosure extends the principles of RTCC by providing combinations of multispecific binding molecules (“MBMs”) useful for treating proliferative diseases and autoimmune disorders. Such combinations typically comprise a MBM (e.g., a bispecific binding molecule (“BBM”) or a trispecific binding molecule (“TBM”)) that engages (a) human CD2 and (b) a human tumor associated antigen (“TAA”) and/or a tumor microenvironment antigen (“TMEA”) and a MBM (e.g., a BBM or TBM) that engages (a) a component of a TCR complex on T-cells or a secondary T-cell signaling molecule and (b) a TAA and/or a TMEA. Without being bound by theory, the inventors believe that engaging two separate pathways in the same immune synapse using these two types of MBMs can induce T-cell proliferation and potentially overcome anergy.

For convenience, a MBM that engages (a) human CD2 and (b) a TAA and/or a TMEA is referred to herein as a “first MBM” and a MBM that engages (a) a component of a TCR complex or a secondary T-cell signaling molecule and (b) a TAA and/or a TMEA is referred to herein as a “second MBM.”

In one aspect, the present disclosure provides first MBMs (e.g., BBMs and TBMs), e.g., for use in combination with second MBMs, comprising (i) an antigen-binding module that binds specifically to human CD2 (referred to herein as “ABM1”) and (ii) an antigen-binding module that binds specifically to a TAA (referred to herein as “ABM2”) and/or an antigen-binding module that binds specifically to a TMEA (referred to herein as “ABM3”).

In another aspect, the present disclosure provides second MBMs (e.g., BBMs and TBMs), e.g., for use in combination with first MBMs, comprising (i) an antigen-binding module that binds specifically to a component of TCR complex or a secondary T-cell signaling molecule (referred to herein as “ABM4”) and (ii) an antigen-binding module that binds specifically to a TAA (referred to herein as “ABM5”) and/or an antigen-binding module that binds specifically to a TMEA (referred to herein as “ABM6”).

In another aspect, the present disclosure provides combinations of a first MBM and a second MBM. Such combinations can be used used, for example, to treat a subject having a proliferative disease or an autoimmune disorder.

In some embodiments, each ABM of a MBM of the disclosure or combination of MBMs is capable of binding its respective target at the same time as each of the other antigen-binding modules of the MBM or combination of MBMs is bound to its respective target. Each of ABM1, ABM2, ABM3, ABM4, ABM5, and ABM6 can be immunoglobulin- or non-immunoglobulin-based. Therefore, the MBMs (e.g., BBMs and TBMs) can include immunoglobulin-based ABMs or any combination of immunoglobulin- and non-immunoglobulin-based ABMs.

Immunoglobulin-based ABMs that can be used in the MBMs (e.g., BBMs and TBMs) are described in Section 7.2.1 and specific embodiments 42-47, 62-67, 274-279, 319-324, 328-333, 841-848, and 852-857 infra. Non-immunoglobulin-based ABMs that can be used in the MBMs (e.g., BBMs and TBMs) are described in Section 7.2.2 and specific embodiments 3-41, 60-61, 272-273, 317-318, 326-327, 841-842, 850-851, infra. Further features of exemplary ABMs that bind to human CD2 are described in Section 7.6 and specific embodiments 5-58, infra. Further features of exemplary ABMs that bind to a TAA are described in Section 7.7 and specific embodiments 334-838 and 907-908, infra. Further features of exemplary ABMs that bind to a TMEA are described in Section 7.8 and specific embodiments 858-902, infra. Further features of exemplary ABMs that bind to a component of a TCR complex are described in Section 7.9 and specific embodiments 68-270, infra. Further features of exemplary ABMs that bind to a secondary T-cell signaling molecule are described in Section 7.10 and specific embodiments 280-314, infra. The ABMs of a MBM (e.g., BBM or TBM) (or portions thereof) can be connected to each other, for example, by short peptide linkers or by an Fc domain. Methods and components for connecting ABMs to form a MBM are described in Section 7.3 and specific embodiments 909-1125, infra.

BBMs have at least two ABMs (e.g., a BBM is at least bivalent), but can also have more than two ABMs. For example, a BBM of the disclosure can have three ABMs (i.e., is trivalent) or four ABMs (i.e., is tetravalent), provided that a first BBM has at least one ABM1 and at least one ABM2 or ABM3 and a second BBM has at least one ABM4 and a least one ABM5 or ABM6. Exemplary bivalent, trivalent, and tetravalent BBM configurations are shown in FIGS. 1B-1H and described in Section 7.4 and specific embodiments 1194-1257, infra.

A TBM can have three ABMs (i.e., is trivalent), four ABMs (i.e., is tetravalent), five ABMs (i.e., is pentavalent), or six ABMs (i.e., is hexavalent). A first TBM can have at least one ABM1, at least one ABM2, and at least one ABM3, and a second TBM can have at least one ABM4, at least one ABM5, and at least one ABM6. Exemplary trivalent, tetravalent, pentavalent, and hexavalent TBM configurations are shown in FIGS. 2B-2V and described in Section 7.5 and specific embodiments 1258-1296, infra.

The disclosure further provides nucleic acids encoding the MBMs (either in a single nucleic acid or a plurality of nucleic acids) and recombinant host cells and cell lines engineered to express the nucleic acids and MBMs of the disclosure. Exemplary nucleic acids, host cells, and cell lines are described in Section 7.11 and specific embodiments 1551-1557, infra.

The present disclosure further provides drug conjugates comprising the MBMs of the disclosure. Such conjugates are referred to herein as “antibody-drug conjugates” or “ADCs” for convenience, notwithstanding that some of the ABMs can be non-immunoglobulin domains. Examples of ADCs are described in Section 7.12 and specific embodiments 1331-1369, infra.

Preparations and pharmaceutical compositions comprising the MBMs and ADCs are also provided. Examples of preparations and pharmaceutical compositions are described in Section 7.13 and specific embodiment 1550, infra.

Further provided herein are methods of using combinations of MBMs, ADCs, and pharmaceutical compositions of the disclosure, for example for treating proliferative conditions (e.g., cancers), on which TAA targeted by the MBMs are expressed, and for treating autoimmune disorders. Exemplary methods are described in Section 7.14 and specific embodiments 1-1542, infra.

The disclosure further provides methods of using the MBMs, the ADCs, and the pharmaceutical compositions in combination with other agents and therapies. Exemplary agents, therapies, and methods of combination therapy are described in Section 7.15 and specific embodiment 1543.

### 6. BRIEF DESCRIPTION OF THE FIGURES

FIGS. 1A-1AH: Exemplary BBM configurations. FIG. 1A illustrates components of the exemplary BBM configurations illustrated in FIGS. 1B-1AH. Not all regions connecting the different domains of each chain are illustrated (e.g., the linker connecting the VH and VL domains of an scFv, the hinge connecting the CH2 and CH3 domains of an Fc domain, etc., are omitted). FIGS. 1B-1F illustrate bivalent BBMs; FIGS. 1G-1Z illustrate trivalent BBMs; FIGS. 1AA-1AH illustrate tetravalent BBMs.

FIGS. 2A-2V: Exemplary TBM configurations. FIG. 2A illustrates components of the exemplary TBM configurations illustrated in FIGS. 2B-2V. Not all regions connecting the different domains of each chain are illustrated (e.g., the linker connecting the VH and VL domains of an scFv, the hinge connecting the CH2 and CH3 domains of an Fc, etc., are omitted). FIG. 2B-2P illustrates trivalent TBMs; FIGS. 2Q-2S illustrate tetravalent TBMs; FIG. 2T illustrates a pentavalent TBM, and FIGS. 2U-2V illustrate hexavalent TBMs.

FIGS. 3A-3D: Effects of CD28 or CD2 engagement on T cell proliferation (FIG. 3A) and cytokine secretion (FIG. 3B: IL2; FIG. 3C: IFNg; FIG. 3D: TNFa) in the presence or absence of CD3 stimulation with anti-CD3 antibody. “CD28” refers to anti-CD28 antibody, “CD3” refers to anti-CD3 antibody, “hCD58-Fc” refers to CD58-Fc protein, and “ISO” refers to isotype control antibody.

FIGS. 4A-4B: Effects of a CD2×CD20 BBM on CD3×CD19 BBM-induced T cell activation in a NFAT Jurkat reporter assay. FIG. 4A: NFAT reporter activity with serial dilutions of CD3×CD19 BBM in the absence or presence of CD2×CD20 BBM. FIG. 4B: NFAT reporter activity of CD2×CD20 BBM in the absence or presence of CD3×CD19 BBM. “CD2×CD20” refers to CD2×CD20 BBM and “CD3×CD19” refers to CD3×CD19 BBM.

FIGS. 5A-5B: Effects of a CD2×CD20 BBM on a CD3×CD19 BBM-induced tumor cell killing assay. FIG. 5A: Tumor cell killing activity with serial dilutions of CD3×CD19 BBM in the absence or presence of CD2×CD20 BBM. FIG. 5B: Tumor cell killing activity with serial dilutions of CD3×CD20 BBM in the absence or presence of CD3×CD19 BBM. “CD2×CD20” refers to CD2×CD20 BBM, “CD3×CD19” refers to CD3×CD19 BBM, and “TSP” refers to CD3×CD19×CD2 TBM.

### 7. DETAILED DESCRIPTION

### 7.1. Definitions

As used herein, the following terms are intended to have the following meanings:

ABM chain: Individual ABMs can exist as one (e.g., in the case of an scFv) polypeptide chain or form through the association of more than one polypeptide chains (e.g., in the case of a Fab). As used herein, the term “ABM chain” refers to all or a portion of an ABM that exists on a single polypeptide chain. The use of the term “ABM chain” is intended for convenience and descriptive purposes only and does not connote a particular configuration or method of production.

ADCC: By “ADCC” or “antibody dependent cell-mediated cytotoxicity” as used herein is meant the cell-mediated reaction where nonspecific cytotoxic cells that express FcγRs recognize bound antibody on a target cell and subsequently cause lysis of the target cell. ADCC is correlated with binding to FcγRIIIa; increased binding to FcγRIIIa leads to an increase in ADCC activity.

ADCP: By “ADCP” or antibody dependent cell-mediated phagocytosis as used herein is meant the cell-mediated reaction where nonspecific phagocytic cells that express FcγRs recognize bound antibody on a target cell and subsequently cause phagocytosis of the target cell.

Additional Agent: For convenience, an agent that is used in combination with one or more MBMs is referred to herein as an “additional” agent.

Antibody: The term “antibody” as used herein refers to a polypeptide (or set of polypeptides) of the immunoglobulin family that is capable of binding an antigen non-covalently, reversibly and specifically. For example, a naturally occurring “antibody” of the IgG type is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain (abbreviated herein as CL). The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system. The term “antibody” includes, but is not limited to, monoclonal antibodies, human antibodies, humanized antibodies, camelised antibodies, chimeric antibodies, bispecific or multispecific antibodies and anti-idiotypic (anti-Id) antibodies (including, e.g., anti-Id antibodies to antibodies of the disclosure). The antibodies can be of any isotype/class (e.g., IgG, IgE, IgM, IgD, IgA and IgY) or subclass (e.g., IgG1, IgG2, IgG3, IgG4, IgA1 and IgA2).

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen-binding site or amino-terminus of the antibody. The N-terminus is a variable region and at the C-terminus is a constant region; the CH3 and CL domains actually comprise the carboxy-terminus of the heavy and light chain, respectively.

Antibody fragment: The term “antibody fragment” of an antibody as used herein refers to one or more portions of an antibody. In some embodiments, these portions are part of the contact domain(s) of an antibody. In some other embodiments, these portion(s) are antigen-binding fragments that retain the ability of binding an antigen non-covalently, reversibly and specifically, sometimes referred to herein as the “antigen-binding fragment”, “antigen-binding fragment thereof,” “antigen-binding portion”, and the like. Examples of binding fragments include, but are not limited to, single-chain Fvs (scFv), a Fab fragment, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., (1989) Nature 341:544-546), which consists of a VH domain; and an isolated complementarity determining region (CDR). Thus, the term “antibody fragment” encompasses both proteolytic fragments of antibodies (e.g., Fab and F(ab)2 fragments) and engineered proteins comprising one or more portions of an antibody (e.g., an scFv).

Antibody fragments can also be incorporated into single domain antibodies, maxibodies, minibodies, intrabodies, diabodies, triabodies, tetrabodies, v-NAR and bis-scFv (see, e.g., Hollinger and Hudson, 2005, Nature Biotechnology 23: 1126-1136). Antibody fragments can be grafted into scaffolds based on polypeptides such as Fibronectin type III (Fn3) (see U.S. Pat. No. 6,703,199, which describes fibronectin polypeptide monobodies).

Antibody fragments can be incorporated into single chain molecules comprising a pair of tandem Fv segments (for example, VH-CH1-VH-CH1) which, together with complementary light chain polypeptides (for example, VL-VC-VL-VC), form a pair of antigen-binding regions (Zapata et al., 1995, Protein Eng. 8:1057-1062; and U.S. Pat. No. 5,641,870).

Antibody Numbering System: In the present specification, the references to numbered amino acid residues in antibody domains are based on the EU numbering system unless otherwise specified. This system was originally devised by Edelman et al., 1969, Proc. Nat'l Acad. Sci. USA 63:78-85 and is described in detail in Kabat et al., 1991, in Sequences of Proteins of Immunological Interest, US Department of Health and Human Services, NIH, USA.

Antigen-binding module: The term “antigen-binding module” or “ABM” as used herein refers to a portion of a MBM that has the ability to bind to an antigen non-covalently, reversibly and specifically. An ABM can be immunoglobulin- or non-immunoglobulin-based. As used herein, the terms “ABM1” and “CD2 ABM” (and the like) refer to an ABM that binds specifically to human CD2, the terms “ABM2” and “TAA 1 ABM” (and the like) refer to an ABM that binds specifically to a human tumor-associated antigen, the term “ABM3” and “TMEA 1 ABM” (and the like) refer to an ABM that binds specifically to a human tumor microenvironment antigen, the term “ABM4” refers to an ABM that binds specifically to a component of a human T-cell receptor (TCR) complex or a secondary T-cell signaling molecule, the term “ABM5” and “TAA 2 ABM” (and the like) refer to an ABM that binds specifically to a human tumor-associated antigen, the term “ABM6” and “TMEA 2 ABM” (and the like) refer to an ABM that binds specifically to a human tumor microenvironment antigen, and the term “TCR ABM” refers to an ABM4 that binds specifically to a component of a TCR complex. The terms ABM1, ABM2, ABM3, ABM4, ABM5, and ABM6 are used merely for convenience and are not intended to convey any particular configuration of a MBM. In some embodiments, an ABM4 binds to CD3 (referred to herein a “CD3 ABM” or the like). Accordingly, disclosures relating to ABM4s and TCR ABMs are also applicable to CD3 ABMs.

Antigen-binding domain: The term “antigen-binding domain” (ABD) refers to a portion of a molecule that has the ability to bind to an antigen non-covalently, reversibly and specifically. Exemplary antigen-binding domains include antigen-binding fragments and portions of both immunoglobulin and non-immunoglobulin based scaffolds that retain the ability of binding an antigen non-covalently, reversibly and specifically. As used herein, the term “antigen-binding domain” encompasses antibody fragments that retain the ability of binding an antigen non-covalently, reversibly and specifically.

Antigen-binding fragment: The term “antigen-binding fragment” of an antibody refers to a portion of an antibody that retains has the ability to bind to an antigen non-covalently, reversibly and specifically.

Associated: The term “associated” in the context of a MBM refers to a functional relationship between two or more polypeptide chains. In particular, the term “associated” means that two or more polypeptides are associated with one another, e.g., non-covalently through molecular interactions or covalently through one or more disulfide bridges or chemical cross-linkages, so as to produce a functional MBM (e.g., a BBM) in which the ABMs of the MBM can bind their respective targets. Examples of associations that might be present in a MBM include (but are not limited to) associations between Fc regions in an Fc domain (homodimeric or heterodimeric as described in Section 7.3.1.5), associations between VH and VL regions in a Fab or Fv, and associations between CH1 and CL in a Fab.

B cell: As used herein, the term “B cell” refers to a cell of B cell lineage, which is a type of white blood cell of the lymphocyte subtype. Examples of B cells include plasmablasts, plasma cells, lymphoplasmacytoid cells, memory B cells, follicular B cells, marginal zone B cells, B-1 cells, B-2 cells, and regulatory B cells.

B cell malignancy: As used herein, a B cell malignancy refers to an uncontrolled proliferation of B cells. Examples of B cell malignancy include non-Hodgkin's lymphomas (NHL), Hodgkin's lymphomas, leukemia, and myeloma. For example, a B cell malignancy can be, but is not limited to, multiple myeloma, chronic lymphocytic leukemia (CLL)/small lymphocytic lymphoma (SLL), follicular lymphoma, mantle cell lymphoma (MCL), diffuse large B-cell lymphoma (DLBCL), marginal zone lymphomas, Burkitt lymphoma, lymphoplasmacytic lymphoma (Waldenstrom macroglobulinemia), hairy cell leukemia, primary central nervous system (CNS) lymphoma, primary mediastinal large B-cell lymphoma, mediastinal grey-zone lymphoma (MGZL), splenic marginal zone B-cell lymphoma, extranodal marginal zone B-cell lymphoma of MALT, nodal marginal zone B-cell lymphoma, and primary effusion lymphoma, and plasmacytic dendritic cell neoplasms.

BCMA: As used herein, the term “BCMA” refers to B-cell maturation antigen. BCMA (also known as TNFRSF17, BCM or CD269) is a member of the tumor necrosis receptor (TNFR) family and is predominantly expressed on terminally differentiated B cells, e.g., memory B cells and plasma cells. Its ligands include B-cell activating factor (BAFF) and a proliferation-inducing ligand (APRIL). The protein BCMA is encoded by the gene TNFRSF17. Exemplary BCMA sequences are available at the Uniprot database under accession number Q02223.

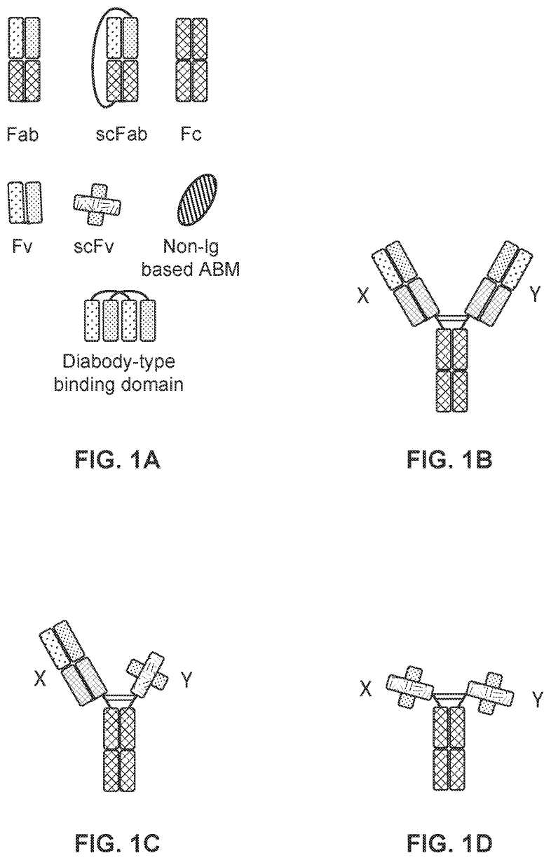

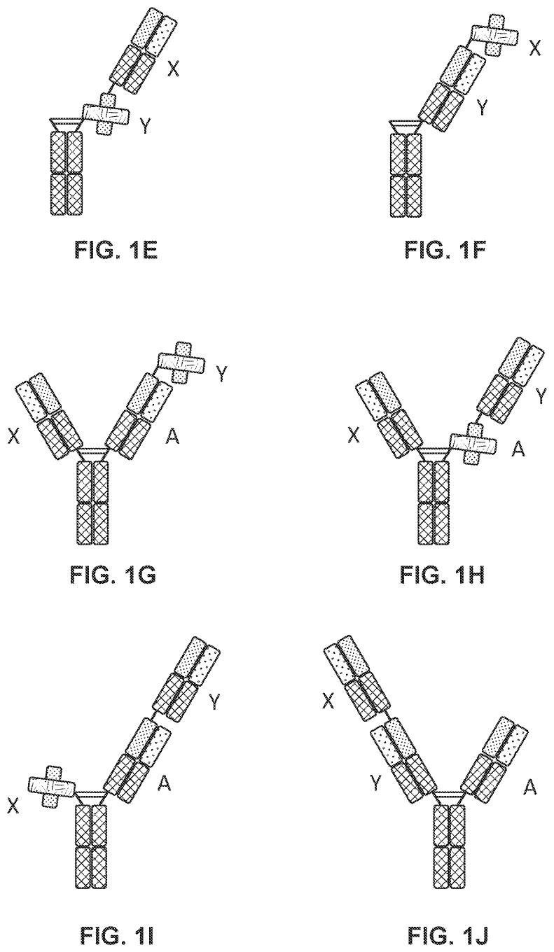

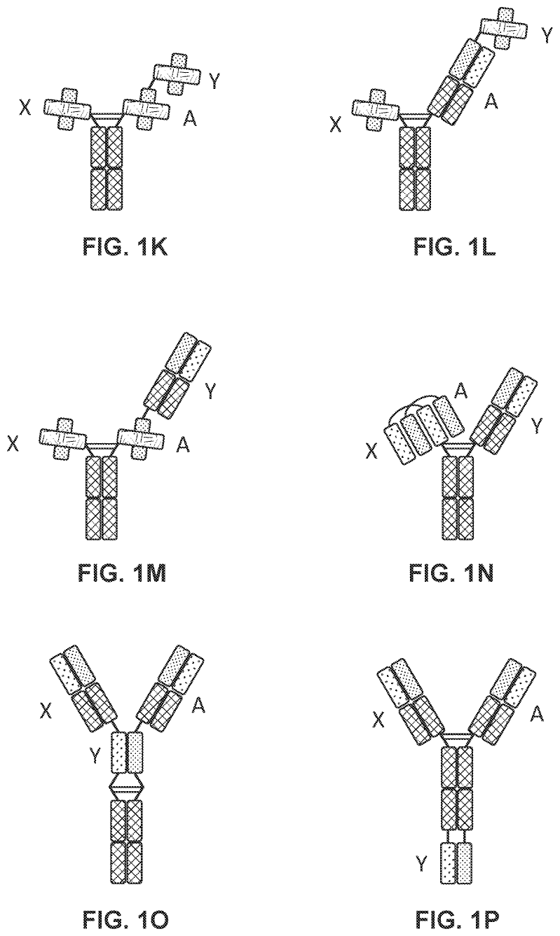

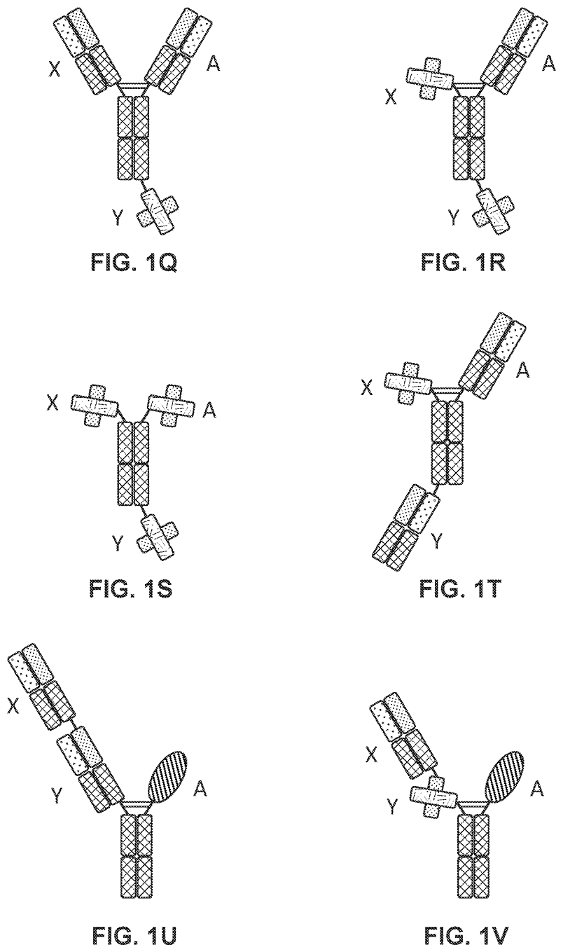

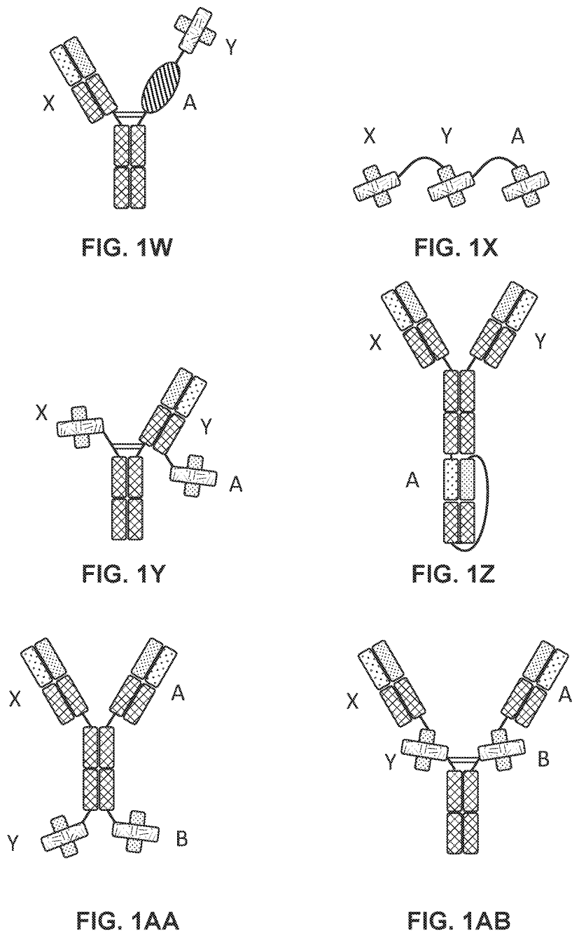

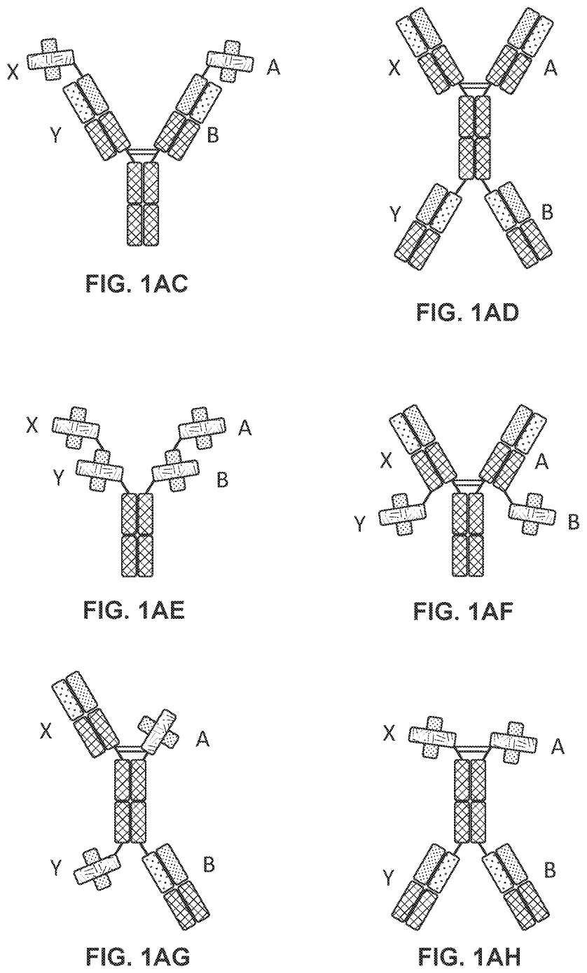

Bispecific binding molecule: The term “bispecific binding molecule” or “BBM” refers to a molecule that specifically binds to two antigens and comprises two or more ABMs. Representative BBMs are illustrated in FIG. 1B-1AH. BBMs can comprise one, two, three, four or even more polypeptide chains.

Binding Sequences: In reference to Tables 11, 14-20, and 22-23 (including subparts thereof), the term “binding sequences” means an ABM having a full set of CDRs, a VH-VL pair, or an scFv set forth in that table.

Bivalent: The term “bivalent” as used herein in the context of an antigen-binding molecule (e.g., a BBM) refers to an antigen-binding molecule that has two antigen-binding domains.

Cancer: The term “cancer” refers to a disease characterized by the uncontrolled (and often rapid) growth of aberrant cells. Cancer cells can spread locally or through the bloodstream and lymphatic system to other parts of the body. Examples of various cancers are described herein and include but are not limited to, hematological cancers such as lymphomas, leukemias, and multiple myeloma, and non-hematological cancers such as ovarian cancer, lung cancer, gastric cancer, breast cancer, hepatic cancer, pancreatic cancer, skin cancer, malignant melanoma, head and neck cancer, sarcoma, bile duct cancer, cancer of the urinary bladder, kidney cancer, colon cancer, placental choriocarcinoma, cervical cancer, testicular cancer, and uterine cancer.

CD3: The term “CD3” or “cluster of differentiation 3” refers to the cluster of differentiation 3 co-receptor of the T cell receptor. CD3 helps in activation of both cytotoxic T-cell (e.g., CD8+ naïve T cells) and T helper cells (e.g., CD4+ naïve T cells) and is composed of four distinct chains: one CD3γ chain (e.g., Genbank Accession Numbers NM_000073 and MP_000064 (human)), one CD3δ chain (e.g., Genbank Accession Numbers NM_000732, NM_001040651, NP_00732 and NP_001035741 (human)), and two CD3c chains (e.g., Genbank Accession Numbers NM_000733 and NP_00724 (human)). The chains of CD3 are highly related cell-surface proteins of the immunoglobulin superfamily containing a single extracellular immunoglobulin domain. The CD3 molecule associates with the T-cell receptor (TCR) and chain to form the T-cell receptor (TCR) complex, which functions in generating activation signals in T lymphocytes.

Unless expressly indicated otherwise, the reference to CD3 in the application can refer to the CD3 co-receptor, the CD3 co-receptor complex, or any polypeptide chain of the CD3 co-receptor complex.

Chimeric Antibody: The term “chimeric antibody” (or antigen-binding fragment thereof) is an antibody molecule (or antigen-binding fragment thereof) in which (a) the constant region, or a portion thereof, is altered, replaced or exchanged so that the antigen-binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. For example, a mouse antibody can be modified by replacing its constant region with the constant region from a human immunoglobulin. Due to the replacement with a human constant region, the chimeric antibody can retain its specificity in recognizing the antigen while having reduced antigenicity in human as compared to the original mouse antibody.

In combination: Administered “in combination,” as used herein, means that two (or more) different treatments (e.g., two BBMs as described herein) are delivered to the subject during the course of the subject's affliction with the disorder, e.g., the two or more treatments are delivered after the subject has been diagnosed with the disorder and before the disorder has been cured or eliminated or treatment has ceased for other reasons.

Complementarity Determining Reaction: The terms “complementarity determining region” or “CDR,” as used herein, refer to the sequences of amino acids within antibody variable regions which confer antigen specificity and binding affinity. For example, in general, there are three CDRs in each heavy chain variable region (e.g., CDR-H1, CDR-H2, and CDR-H3) and three CDRs in each light chain variable region (CDR-L1, CDR-L2, and CDR-L3). The precise amino acid sequence boundaries of a given CDR can be determined using any of a number of well-known schemes, including those described by Kabat et al., 1991, “Sequences of Proteins of Immunological Interest,” 5th Ed. Public Health Service, National Institutes of Health, Bethesda, Md. (“Kabat” numbering scheme), Al-Lazikani et al., 1997, JMB 273:927-948 (“Chothia” numbering scheme) and ImMunoGenTics (IMGT) numbering (Lefranc, 1999, The Immunologist 7:132-136 (1999); Lefranc et al., 2003, Dev. Comp. Immunol. 27:55-77 (“IMGT” numbering scheme). For example, for classic formats, under Kabat, the CDR amino acid residues in the heavy chain variable domain (VH) are numbered 31-35 (CDR-H1), 50-65 (CDR-H2), and 95-102 (CDR-H3); and the CDR amino acid residues in the light chain variable domain (VL) are numbered 24-34 (CDR-L1), 50-56 (CDR-L2), and 89-97 (CDR-L3). Under Chothia, the CDR amino acids in the VH are numbered 26-32 (CDR-H1), 52-56 (CDR-H2), and 95-102 (CDR-H3); and the amino acid residues in VL are numbered 26-32 (CDR-L1), 50-52 (CDR-L2), and 91-96 (CDR-L3). By combining the CDR definitions of both Kabat and Chothia, the CDRs consist of amino acid residues 26-35 (CDR-H1), 50-65 (CDR-H2), and 95-102 (CDR-H3) in human VH and amino acid residues 24-34 (CDR-L1), 50-56 (CDR-L2), and 89-97 (CDR-L3) in human VL. Under IMGT the CDR amino acid residues in the VH are numbered approximately 26-35 (CDR-H1), 51-57 (CDR-H2) and 93-102 (CDR-H3), and the CDR amino acid residues in the VL are numbered approximately 27-32 (CDR-L1), 50-52 (CDR-L2), and 89-97 (CDR-L3) (numbering according to “Kabat”). Under IMGT, the CDR regions of an antibody can be determined using the program IMGT/DomainGap Align.

Concurrently: The term “concurrently” is not limited to the administration of therapies (e.g., prophylactic or therapeutic agents) at exactly the same time, but rather it is meant that a pharmaceutical composition comprising a MBM or ADC is administered to a subject in a sequence and within a time interval such that the molecules can act together with the additional therapy(ies) to provide an increased benefit than if they were administered otherwise.

Conservative Sequence Modifications: The term “conservative sequence modifications” refers to amino acid modifications that do not significantly affect or alter the binding characteristics of a MBM or a component thereof (e.g., an ABM or an Fc region). Such conservative modifications include amino acid substitutions, additions and deletions. Modifications can be introduced into a MBM by standard techniques, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a MBM can be replaced with other amino acid residues from the same side chain family and the altered MBM can be tested for, e.g., binding to target molecules and/or effective heterodimerization and/or effector function.

Diabody: The term “diabody” as used herein refers to small antibody fragments with two antigen-binding sites, typically formed by pairing of scFv chains. Each scFv comprises a heavy chain variable domain (VH) connected to a light chain variable domain (VL) in the same polypeptide chain (VH-VL, where the VH is either N-terminal or C-terminal to the VL). Unlike a typical scFv in which the VH and VL are separated by a linker that allows the VH and VL on the same polypeptide chain to pair and form an antigen-binding domain, diabodies typically comprise a linker that is too short to allow pairing between the VH and VL domains on the same chain, forcing the VH and VL domains to pair with the complementary domains of another chain and create two antigen-binding sites. Diabodies are described more fully in, for example, EP 404,097; WO 93/11161; and Hollinger et al., 1993, Proc. Natl. Acad. Sci. USA 90:6444-6448.

dsFv: The term “dsFv” refers to disulfide-stabilized Fv fragments. In a dsFv, a VH and VL are connected by an interdomain disulfide bond. To generate such molecules, one amino acid each in the framework region of in VH and VL are mutated to a cysteine, which in turn form a stable interchain disulfide bond. Typically, position 44 in the VH and position 100 in the VL are mutated to cysteines. See Brinkmann, 2010, Antibody Engineering 181-189, DOI:10.1007/978-3-642-01147-4_14. The term dsFv encompasses both what is as a dsFv (a molecule in which the VH and VL are connected by an interchain disulfide bond but not a linker peptide) or scdsFv (a molecule in which the VH and VL are connected by a linker as well as an interchain disulfide bond).

Effector Function: The term “effector function” refers to an activity of an antibody molecule that is mediated by binding through a domain of the antibody other than the antigen-binding domain, usually mediated by binding of effector molecules. Effector function includes complement-mediated effector function, which is mediated by, for example, binding of the C1 component of the complement to the antibody. Activation of complement is important in the opsonization and lysis of cell pathogens. The activation of complement also stimulates the inflammatory response and may also be involved in autoimmune hypersensitivity. Effector function also includes Fc receptor (FcR)-mediated effector function, which can be triggered upon binding of the constant domain of an antibody to an Fc receptor (FcR). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production. An effector function of an antibody can be altered by altering, e.g., enhancing or reducing, the affinity of the antibody for an effector molecule such as an Fc receptor or a complement component. Binding affinity will generally be varied by modifying the effector molecule binding site, and in this case it is appropriate to locate the site of interest and modify at least part of the site in a suitable way. It is also envisaged that an alteration in the binding site on the antibody for the effector molecule need not alter significantly the overall binding affinity but can alter the geometry of the interaction rendering the effector mechanism ineffective as in non-productive binding. It is further envisaged that an effector function can also be altered by modifying a site not directly involved in effector molecule binding, but otherwise involved in performance of the effector function.

Epitope: An epitope, or antigenic determinant, is a portion of an antigen recognized by an antibody or other antigen-binding moiety as described herein. An epitope can be linear or conformational.

Fab: By “Fab” or “Fab region” as used herein is meant a polypeptide region that comprises the VH, CH1, VL, and CL immunoglobulin domain. These terms can refer to this region in isolation, or this region in the context of an antigen-binding molecule of the disclosure.

Fab domains are formed by association of a CH1 domain attached to a VH domain with a CL domain attached to a VL domain. The VH domain is paired with the VL domain to constitute the Fv region, and the CH1 domain is paired with the CL domain to further stabilize the binding module. A disulfide bond between the two constant domains can further stabilize the Fab domain.

Fab regions can be produced by proteolytic cleavage of immunoglobulin molecules (e.g., using enzymes such as papain) or through recombinant expression. In native immunoglobulin molecules, Fabs are formed by association of two different polypeptide chains (e.g., VH-CH1 on one chain associates with VL-CL on the other chain). The Fab regions are typically expressed recombinantly, typically on two polypeptide chains, although single chain Fabs are also contemplated herein.

Fc domain: The term “Fc domain” refers to a pair of associated Fc regions. The two Fc regions dimerize to create the Fc domain. The two Fc regions within the Fc domain can be the same (such an Fc domain being referred to herein as an “Fc homodimer”) or different from one another (such an Fc domain being referred to herein as an “Fc heterodimer”).

Fc region: The term “Fc region” or “Fc chain” as used herein is meant the polypeptide comprising the CH2-CH3 domains of an IgG molecule, and in some cases, inclusive of the hinge. In EU numbering for human IgG1, the CH2-CH3 domain comprises amino acids 231 to 447, and the hinge is 216 to 230. Thus the definition of “Fc region” includes both amino acids 231-447 (CH2-CH3) or 216-447 (hinge-CH2-CH3), or fragments thereof. An “Fc fragment” in this context can contain fewer amino acids from either or both of the N- and C-termini but still retains the ability to form a dimer with another Fc region as can be detected using standard methods, generally based on size (e.g., non-denaturing chromatography, size exclusion chromatography). Human IgG Fc regions are of particular use in the present disclosure, and can be the Fc region from human IgG1, IgG2 or IgG4.

Fv: The term “Fv” refers to the minimum antibody fragment derivable from an immunoglobulin that contains a complete target recognition and binding site. This region consists of a dimer of one heavy and one light chain variable domain in a tight, noncovalent association (VH-VL dimer). It is in this configuration that the three CDRs of each variable domain interact to define a target binding site on the surface of the VH-VL dimer. Often, the six CDRs confer target binding specificity to the antibody. However, in some instances even a single variable domain (or half of an Fv comprising only three CDRs specific for a target) can have the ability to recognize and bind target. The reference to a VH-VL dimer herein is not intended to convey any particular configuration. By way of example and not limitation, the VH and VL can come together in any configuration described herein to form a half antibody, or can each be present on a separate half antibody and come together to form an antigen binding domain when the separate half antibodies associate, for example to form a BBM of the disclosure. When present on a single polypeptide chain (e.g., a scFv), the VH and be N-terminal or C-terminal to the VL.

Half Antibody: The term “half antibody” refers to a molecule that comprises at least one ABM or ABM chain and can associate with another molecule comprising an ABM or ABM chain through, e.g., a disulfide bridge or molecular interactions (e.g., knob-in-hole interactions between Fc heterodimers). A half antibody can be composed of one polypeptide chain or more than one polypeptide chains (e.g., the two polypeptide chains of a Fab). In an embodiment, a half-antibody comprises an Fc region.

An example of a half antibody is a molecule comprising a heavy and light chain of an antibody (e.g., an IgG antibody). Another example of a half antibody is a molecule comprising a first polypeptide comprising a VL domain and a CL domain, and a second polypeptide comprising a VH domain, a CH1 domain, a hinge domain, a CH2 domain, and a CH3 domain, where the VL and VH domains form an ABM. Yet another example of a half antibody is a polypeptide comprising an scFv domain, a CH2 domain and a CH3 domain.

A half antibody might include more than one ABM, for example a half-antibody comprising (in N- to C-terminal order) an scFv domain, a CH2 domain, a CH3 domain, and another scFv domain.

Half antibodies might also include an ABM chain that when associated with another ABM chain in another half antibody forms a complete ABM.

Thus, a MBM (e.g., a BBM) can comprise one, more typically two, or even more than two half antibodies, and a half antibody can comprise one or more ABMs or ABM chains.

In some MBMs, a first half antibody will associate, e.g., heterodimerize, with a second half antibody. In other MBMs, a first half antibody will be covalently linked to a second half antibody, for example through disulfide bridges or chemical crosslinking. In yet other MBMs, a first half antibody will associate with a second half antibody through both covalent attachments and non-covalent interactions, for example disulfide bridges and knob-in-hole interactions.

The term “half antibody” is intended for descriptive purposes only and does not connote a particular configuration or method of production. Descriptions of a half antibody as a “first” half antibody, a “second” half antibody, a “left” half antibody, a “right” half antibody or the like are merely for convenience and descriptive purposes.

Hole: In the context of a knob-into-hole, a “hole” refers to at least one amino acid side chain which is recessed from the interface of a first Fc chain and is therefore positionable in a compensatory “knob” on the adjacent interfacing surface of a second Fc chain so as to stabilize the Fc heterodimer, and thereby favor Fc heterodimer formation over Fc homodimer formation, for example.

Host cell or recombinant host cell: The terms “host cell” or “recombinant host cell” refer to a cell that has been genetically-engineered, e.g., through introduction of a heterologous nucleic acid. It should be understood that such terms are intended to refer not only to the particular subject cell but to the progeny of such a cell. Because certain modifications can occur in succeeding generations due to either mutation or environmental influences, such progeny may not, in fact, be identical to the parent cell, but are still included within the scope of the term “host cell” as used herein. A host cell can carry the heterologous nucleic acid transiently, e.g., on an extrachromosomal heterologous expression vector, or stably, e.g., through integration of the heterologous nucleic acid into the host cell genome. For purposes of expressing a MBM of the disclosure, a host cell can be a cell line of mammalian origin or mammalian-like characteristics, such as monkey kidney cells (COS, e.g., COS-1, COS-7), HEK293, baby hamster kidney (BHK, e.g., BHK21), Chinese hamster ovary (CHO), NSO, PerC6, BSC-1, human hepatocellular carcinoma cells (e.g., Hep G2), SP2/0, HeLa, Madin-Darby bovine kidney (MDBK), myeloma and lymphoma cells, or derivatives and/or engineered variants thereof. The engineered variants include, e.g., glycan profile modified and/or site-specific integration site derivatives.

Human Antibody: The term “human antibody” as used herein includes antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences or antibody containing consensus framework sequences derived from human framework sequences analysis, for example, as described in Knappik et al., 2000, J Mol Biol 296, 57-86. The structures and locations of immunoglobulin variable domains, e.g., CDRs, can be defined using well known numbering schemes, e.g., the Kabat numbering scheme, the Chothia numbering scheme, or a combination of Kabat and Chothia (see, e.g., Lazikani et al., 1997, J. Mol. Bio. 273:927 948; Kabat et al., 1991, Sequences of Proteins of Immunological Interest, 5th edit., NIH Publication no. 91-3242 U.S. Department of Health and Human Services; Chothia et al., 1987, J. Mol. Biol. 196:901-917; Chothia et al., 1989, Nature 342:877-883).

Human antibodies can include amino acid residues not encoded by human sequences (e.g., mutations introduced by random or site-specific mutagenesis in vitro or by somatic mutation in vivo, or a conservative substitution to promote stability or manufacturing). However, the term “human antibody”, as used herein, is not intended to include antibodies in which CDR sequences derived from the germline of another mammalian species, such as a mouse, have been grafted onto human framework sequences.

Humanized: The term “humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired specificity, affinity, and capacity. In some instances, framework region (FR) residues of the human immunoglobulin are replaced by corresponding non-human residues. Furthermore, humanized antibodies can comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin lo sequence. The humanized antibody optionally will also comprise at least a portion of an immunoglobulin constant region (Fc), typically that of a human immunoglobulin. For further details, see Jones et al., 1986, Nature 321:522-525; Riechmann et al., 1988, Nature 332:323-329; and Presta, 1992, Curr. Op. Struct. Biol. 2:593-596. See also the following review articles and references cited therein: Vaswani and Hamilton, 1998, Ann. Allergy, Asthma & Immunol. 1:105-115; Harris, 1995, Biochem. Soc. Transactions 23:1035-1038; Hurle and Gross, 1994, Curr. Op. Biotech. 5:428-433.

Knob: In the context of a knob-into-hole, a “knob” refers to at least one amino acid side chain which projects from the interface of a first Fc chain and is therefore positionable in a compensatory “hole” in the interface with a second Fc chain so as to stabilize the Fc heterodimer, and thereby favor Fc heterodimer formation over Fc homodimer formation, for example.

Knobs and holes (or knobs-into-holes): One mechanism for Fc heterodimerization is generally referred to in the art as “knobs and holes”, or “knob-in-holes”, or “knobs-into-holes”. These terms refer to amino acid mutations that create steric influences to favor formation of Fc heterodimers over Fc homodimers, as described in, e.g., Ridgway et al., 1996, Protein Engineering 9(7):617; Atwell et al., 1997, J. Mol. Biol. 270:26; and U.S. Pat. No. 8,216,805. Knob-in-hole mutations can be combined with other strategies to improve heterodimerization, for example as described in Section 7.3.1.6.

Monoclonal Antibody: The term “monoclonal antibody” as used herein refers to polypeptides, including antibodies, antibody fragments, molecules (including BBMs), etc. that are derived from the same genetic source.

Multispecific binding molecules: The term “multispecific binding molecules” or “MBMs” refers to molecules that specifically bind to at least two antigens and comprise two or more antigen-binding domains. The antigen-binding domains can each independently be an antibody fragment (e.g., scFv, Fab, nanobody), a ligand, or a non-antibody derived binder (e.g., fibronectin, Fynomer, DARPin).

Mutation or modification: In the context of the primary amino acid sequence of a polypeptide, the terms “modification” and “mutation” refer to an amino acid substitution, insertion, and/or deletion in the polypeptide sequence relative to a reference polypeptide. Additionally, the term “modification” further encompasses an alteration to an amino acid residue, for example by chemical conjugation (e.g., of a drug or polyethylene glycol moiety) or post-translational modification (e.g., glycosylation).

Nucleic Acid: The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, and peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, as detailed below, degenerate codon substitutions can be achieved by generating sequences in which the third position of one or more selected (or all) codons is substituted with mixed-base and/or deoxyinosine residues (Batzer et al., (1991) Nucleic Acid Res. 19:5081; Ohtsuka et al., (1985) J. Biol. Chem. 260:2605-2608; and Rossolini et al., (1994) Mol. Cell. Probes 8:91-98).

Operably linked: The term “operably linked” refers to a functional relationship between two or more peptide or polypeptide domains or nucleic acid (e.g., DNA) segments. In the context of a fusion protein or other polypeptide, the term “operably linked” means that two or more amino acid segments are linked so as to produce a functional polypeptide. For example, in the context of a MBM of the disclosure, separate ABMs (or chains of an ABM) can be through peptide linker sequences. In the context of a nucleic acid encoding a fusion protein, such as a polypeptide chain of a MBM of the disclosure, “operably linked” means that the two nucleic acids are joined such that the amino acid sequences encoded by the two nucleic acids remain in-frame. In the context of transcriptional regulation, the term refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system.

Polypeptide and Protein: The terms “polypeptide” and “protein” are used interchangeably herein to refer to a polymer of amino acid residues. The terms encompass amino acid polymers in which one or more amino acid residue is an artificial chemical mimetic of a corresponding naturally occurring amino acid, as well as to naturally occurring amino acid polymers and non-naturally occurring amino acid polymer. Additionally, the terms encompass amino acid polymers that are derivatized, for example, by synthetic derivatization of one or more side chains or termini, glycosylation, PEGylation, circular permutation, cyclization, linkers to other molecules, fusion to proteins or protein domains, and addition of peptide tags or labels.

Recognize: The term “recognize” as used herein refers to an ABM that finds and interacts (e.g., binds) with its epitope.

Sequence identity: Sequence identity between two similar sequences (e.g., antibody variable domains) can be measured by algorithms such as that of Smith, T. F. & Waterman, M. S. (1981) “Comparison Of Biosequences,” Adv. Appl. Math. 2:482 [local homology algorithm]; Needleman, S. B. & Wunsch, C D. (1970) “A General Method Applicable To The Search For Similarities In The Amino Acid Sequence Of Two Proteins,” J. Mol. Biol. 48:443 [homology alignment algorithm], Pearson, W. R. & Lipman, D. J. (1988) “Improved Tools For Biological Sequence Comparison,” Proc. Natl. Acad. Sci. (U.S.A.) 85:2444 [search for similarity method]; or Altschul, S. F. et al, (1990) “Basic Local Alignment Search Tool,” J. Mol. Biol. 215:403-10, the “BLAST” algorithm, see blast.ncbi.nlm.nih.gov/Blast.cgi. When using any of the aforementioned algorithms, the default parameters (for Window length, gap penalty, etc) are used. In one embodiment, sequence identity is done using the BLAST algorithm, using default parameters.

Optionally, the identity is determined over a region that is at least about 50 nucleotides (or, in the case of a peptide or polypeptide, at least about 10 amino acids) in length, or in some cases over a region that is 100 to 500 or 1000 or more nucleotides (or 20, 50, 200 or more amino acids) in length. In some embodiments, the identity is determined over a defined domain, e.g., the VH or VL of an antibody. Unless specified otherwise, the sequence identity between two sequences is determined over the entire length of the shorter of the two sequences.

Single Chain Fab or scFab: The terms “single chain Fab” and “scFab” mean a polypeptide comprising an antibody heavy chain variable domain (VH), an antibody constant domain 1 (CH1), an antibody light chain variable domain (VL), an antibody light chain constant domain (CL) and a linker, such that the VH and VL are in association with one another and the CH1 and CL are in association with one another. In some embodiments, the antibody domains and the linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL. The linker can be a polypeptide of at least 30 amino acids, for example between 32 and 50 amino acids. The single chain Fabs are stabilized via the natural disulfide bond between the CL domain and the CH1 domain.

Single Chain Fv or scFv: The term “single-chain Fv” or “scFv” as used herein refers to antibody fragments comprise the VH and VL domains of an antibody, where these domains are present in a single polypeptide chain. The Fv polypeptide can further comprise a polypeptide linker between the VH and VL domains which enables the scFv to form the desired structure for antigen-binding. For a review of scFv see Plückthun in The Pharmacology of Monoclonal Antibodies, vol. 113, Rosenburg and Moore eds., (1994) Springer-Verlag, New York, pp. 269-315.

Specifically (or selectively) binds: The term “specifically (or selectively) binds” to an antigen or an epitope refers to a binding reaction that is determinative of the presence of a cognate antigen or an epitope in a heterogeneous population of proteins and other biologics. The binding reaction can be but need not be mediated by an antibody or antibody fragment, but can also be mediated by, for example, any type of ABM described in Section 7.2, such as a ligand, a DARPin, etc. An ABM typically also has a dissociation rate constant (KD) (koff/kon) of less than 5×10⁻²M, less than 10⁻²M, less than 5×10⁻³M, less than 10⁻³M, less than 5×10⁻⁴M, less than 10⁻⁴M, less than 5×10⁻⁵M, less than 10⁻⁵M, less than 5×10⁻⁶M, less than 10⁻⁶M, less than 5×10⁻⁷M, less than 10⁻⁷M, less than 5×10⁻⁸M, less than 10⁻⁸M, less than 5×10⁻⁹M, or less than 10⁻⁹M, and binds to the target antigen with an affinity that is at least two-fold greater than its affinity for binding to a non-specific antigen (e.g., HSA). The term “specifically binds” does not exclude cross-species reactivity. For example, an antigen-binding module (e.g., an antigen-binding fragment of an antibody) that “specifically binds” to an antigen from one species can also “specifically bind” to that antigen in one or more other species. Thus, such cross-species reactivity does not itself alter the classification of an antigen-binding module as a “specific” binder. In certain embodiments, an antigen-binding module (e.g., ABM1, ABM2, ABM3, ABM4, ABM5 and/or ABM6) that specifically binds to a human antigen has cross-species reactivity with one or more non-human mammalian species, e.g., a primate species (including but not limited to one or more of Macaca fascicularis, Macaca mulatta, and Macaca nemestrina) or a rodent species, e.g., Mus musculus. In other embodiments, the antigen-binding module (e.g., ABM1, ABM2, ABM3, ABM4, ABM5, and/or ABM6) does not have cross-species reactivity.

Subject: The term “subject” includes human and non-human animals. Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

Tandem of VH Domains: The term “a tandem of VH domains (or VHs)” as used herein refers to a string of VH domains, consisting of multiple numbers of identical VH domains of an antibody. Each of the VH domains, except the last one at the end of the tandem, has its C-terminus connected to the N-terminus of another VH domain with or without a linker. A tandem has at least 2 VH domains, and in particular embodiments of the MBMs has 3, 4, 5, 6, 7, 8, 9, or 10 VH domains. The tandem of VH can be produced by joining the encoding nucleic acids of each VH domain in a desired order using recombinant methods with or without a linker (e.g., as described in Section 7.3.3) that enables them to be made as a single polypeptide chain. The N-terminus of the first VH domain in the tandem is defined as the N-terminus of the tandem, while the C-terminus of the last VH domain in the tandem is defined as the C-terminus of the tandem.

Tandem of VL Domains: The term “a tandem of VL domains (or VLs)” as used herein refers to a string of VL domains, consisting of multiple numbers of identical VL domains of an antibody. Each of the VL domains, except the last one at the end of the tandem, has its C-terminus connected to the N-terminus of another VL with or without a linker. A tandem has at least 2 VL domains, and in particular embodiments an MBM has 3, 4, 5, 6, 7, 8, 9, or 10 VL domains. The tandem of VL can be produced by joining the encoding nucleic acids of each VL domain in a desired order using recombinant methods with or without a linker (e.g., as described in Section 7.3.3) that enables them to be made as a single polypeptide chain. The N-terminus of the first VL domain in the tandem is defined as the N-terminus of the tandem, while the C-terminus of the last VL domain in the tandem is defined as the C-terminus of the tandem.

Target Antigen: By “target antigen” as used herein is meant the molecule that is bound non-covalently, reversibly and specifically by an antigen binding domain.

Secondary T-cell signaling molecule: The term secondary T cell signaling molecule refers to a cell surface receptor or ligand that engages between T cells and accessory cells, resulting in modulation of the signal generated when the T-cell receptor (“TCR”) recognizes an antigen on accessory cells such as antigen presenting cells (such signal referred to herein as the “primary” T cell signal). Thus, a secondary T cell signaling molecule can inhibit the primary T cell signal, or it can combine with and enhance the response to the primary T-cell signal. One such example of enhancing the response to the primary signal is costimulation. In the absence of costimulation, engagement of the TCR can result in anergy or apoptosis, but when a costimulatory pathway is activated the T cells undergo proliferation and differentiation instead. Exemplary secondary T-cell signaling molecules include CD27, CD28, CD30, CD40L, CD150, CD160, CD226, CD244, BTLA, BTN3A1, B7-1, CTLA4, DR3, GITR, HVEM, ICOS, LAG3, LAIR1, LIGHT, OX40, PD1, PDL1, PDL2, TIGIT, TIM1, TIM2, TIM3, VISTA, CD70, and 4-1BB.

Tetravalent: The term “tetravalent” refers to MBM (e.g., a BBM) that has four ABMs. In certain embodiments, tetravalent BBMs generally have two ABMs that each specifically bind to the first antigen and two ABMs that each specifically bind to the second antigen, although other configurations are contemplated whereby three ABMs specifically bind to one antigen and one ABM specifically binds to a different antigen. Examples of tetravalent configurations are shown schematically in FIGS. 1AA-1AH.

Therapeutically effective amount: A “therapeutically effective amount” refers to an amount effective, at dosages and for periods of time necessary, to achieve a desired therapeutic result.

Treat, Treatment, Treating: As used herein, the terms “treat”, “treatment” and “treating” refer to the reduction or amelioration of the progression, severity and/or duration of a disease or disorder (e.g., a proliferative disorder), or the amelioration of one or more symptoms (e.g., one or more discernible symptoms) of a disorder resulting from the administration of one or more MBMs (e.g., BBMs) of the disclosure. In some embodiments, the terms “treat”, “treatment” and “treating” refer to the amelioration of at least one measurable physical parameter of a disorder, such as growth of a tumor, not necessarily discernible by the patient. In other embodiments the terms “treat”, “treatment” and “treating” refer to the inhibition of the progression of a disorder, either physically by, e.g., stabilization of a discernible symptom, physiologically by, e.g., stabilization of a physical parameter, or both. In some embodiments, the terms “treat”, “treatment” and “treating” can refer to the reduction or stabilization of tumor size or cancerous cell count.

Trivalent: The term “trivalent” refers to MBM (e.g., a BBM) that has three ABMs. Trivalent BBMs have two ABMs that bind to one antigen and one ABM that binds to a different antigen. Examples of trivalent configurations are shown schematically in FIGS. 1G-1Z.

Tumor: The term “tumor” is used interchangeably with the term “cancer” herein, e.g., both terms encompass solid and liquid, e.g., diffuse or circulating, tumors. As used herein, the term “cancer” or “tumor” includes premalignant, as well as malignant cancers and tumors.

Tumor-Associated Antigen: The term “tumor-associated antigen” or “TAA” refers to a molecule (typically a protein, carbohydrate, lipid or some combination thereof) that is expressed on the surface of a cancer cell, either entirely or as a fragment (e.g., MHC/peptide), and which is useful for the preferential targeting of a pharmacological agent to the cancer cell. In some embodiments, a TAA is a marker expressed by both normal cells and cancer cells, e.g., a lineage marker, e.g., CD19 on B cells. In some embodiments, a TAA is a cell surface molecule that is overexpressed in a cancer cell in comparison to a normal cell, for instance, 1-fold over expression, 2-fold overexpression, 3-fold overexpression or more in comparison to a normal cell. In some embodiments, a TAA is a cell surface molecule that is inappropriately synthesized in the cancer cell, for instance, a molecule that contains deletions, additions or mutations in comparison to the molecule expressed on a normal cell. In some embodiments, a TAA will be expressed exclusively on the cell surface of a cancer cell, entirely or as a fragment (e.g., MHC/peptide), and not synthesized or expressed on the surface of a normal cell. Accordingly, the term “TAA” encompasses antigens that are specific to cancer cells, sometimes known in the art as tumor-specific antigens (“TSAs”).

Tumor Microenvironment Antigen: The term “tumor microenvironment antigen” or “TMEA” refers to a molecule (typically a protein, carbohydrate, lipid or some combination thereof) that is expressed on the surface of a non-cancer cell in a tumor microenvironment or secreted by a cell in the tumor microenvironment, either entirely or as a fragment (e.g., MHC/peptide), and which is useful for the preferential targeting of a pharmacological agent to the tumor microenvironment. In some embodiments, a TMEA is a cell surface molecule that is overexpressed in a tumor microenvironment in comparison to outside of the tumor microenvironment, for instance, 1-fold over expression, 2-fold overexpression, 3-fold overexpression or more in comparison to outside of the tumor microenvironment.

Variable region: By “variable region” or “variable domain” as used herein is meant the region of an immunoglobulin that comprises one or more Ig domains substantially encoded by any of the Vκ, Vλ, and/or VH genes that make up the kappa, lambda, and heavy chain immunoglobulin genetic loci respectively, and contains the CDRs that confer antigen specificity. A “variable heavy domain” can pair with a “variable light domain” to form an antigen binding domain (“ABD”). In addition, each variable domain comprises three hypervariable regions (“complementary determining regions,” “CDRs”) (CDR-H1, CDR-H2, CDR-H3 for the variable heavy domain and CDR-L1, CDR-L2, CDR-L3 for the variable light domain) and four framework (FR) regions, arranged from amino-terminus to carboxy-terminus in the following order: FR1-CDR1-FR2-CDR2-FR3-CDR3-FR4.

Vector: The term “vector” is intended to refer to a polynucleotide molecule capable of transporting another polynucleotide to which it has been linked. One type of vector is a “plasmid”, which refers to a circular double stranded DNA loop into which additional DNA segments can be ligated. Another type of vector is a viral vector, where additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operably linked. Such vectors are referred to herein as “recombinant expression vectors” (or simply, “expression vectors”). In general, expression vectors of utility in recombinant DNA techniques are often in the form of plasmids. In the present specification, “plasmid” and “vector” can be used interchangeably as the plasmid is the most commonly used form of vector. However, the disclosure is intended to include such other forms of expression vectors, such as viral vectors (e.g., replication defective retroviruses, adenoviruses and adeno-associated viruses), which serve equivalent functions.

VH: The term “VH” refers to the variable region of an immunoglobulin heavy chain of an antibody, including the heavy chain of an Fv, scFv, dsFv or Fab.

VL: The term “VL” refers to the variable region of an immunoglobulin light chain, including the light chain of an Fv, scFv, dsFv or Fab.

VH-VL or VH-VL Pair: In reference to a VH-VL pair, whether on the same polypeptide chain or on different polypeptide chains, the terms “VH-VL” and “VH-VL pair” are used for convenience and are not intended to convey any particular orientation, unless the context dictates otherwise. Thus, a scFv comprising a “VH-VL” or “VH-VL pair” can have the VH and VL domains in any orientation, for example the VH N-terminal to the VL or the VL N-terminal to the VH.

### 7.2. Antigen Binding Modules

Typically, one or more ABMs of the MBMs comprise immunoglobulin-based antigen-binding domains, for example the sequences of antibody fragments or derivatives. These antibody fragments and derivatives typically include the CDRs of an antibody and can include larger fragments and derivatives thereof, e.g., Fabs, scFabs, Fvs, and scFvs.

Immunoglobulin-based ABMs can comprise modifications to framework residues within a VH and/or a VL, e.g. to improve the properties of a MBM containing the ABM. For example, framework modifications can be made to decrease immunogenicity of a MBM. One approach for making such framework modifications is to “back-mutate” one or more framework residues of the ABM to a corresponding germline sequence. Such residues can be identified by comparing framework sequences to germline sequences from which the ABM is derived. To “match” framework region sequences to desired germline configuration, residues can be “back-mutated” to a corresponding germline sequence by, for example, site-directed mutagenesis. MBMs having such “back-mutated” ABMs are intended to be encompassed by the disclosure.

Another type of framework modification involves mutating one or more residues within a framework region, or even within one or more CDR regions, to remove T-cell epitopes to thereby reduce potential immunogenicity of a MBM. This approach is also referred to as “deimmunization” and is described in further detail in U.S. Patent Publication No. 20030153043 by Carr et al.

ABMs can also be modified to have altered glycosylation, which can be useful, for example, to increase the affinity of a MBM for one or more of its antigens. Such carbohydrate modifications can be accomplished by, for example, altering one or more sites of glycosylation within an ABM sequence. For example, one or more amino acid substitutions can be made that result in elimination of one or more variable region framework glycosylation sites to thereby eliminate glycosylation at that site. Such aglycosylation may increase the affinity of the MBM for an antigen. Such an approach is described in, e.g., U.S. Pat. Nos. 5,714,350 and 6,350,861 by Co et al.

### 7.2.1. Immunoglobulin Based Modules

### 7.2.1.1. Fabs

In certain aspects, an ABM is a Fab domain. Fab domains can be produced by proteolytic cleavage of immunoglobulin molecules, using enzymes such as papain, or through recombinant expression. Fab domains typically comprise a CH1 domain attached to a VH domain which pairs with a CL domain attached to a VL domain.

In a wild-type immunoglobulin, the VH domain is paired with the VL domain to constitute the Fv region, and the CH1 domain is paired with the CL domain to further stabilize the binding module. A disulfide bond between the two constant domains can further stabilize the Fab domain.

For the MBMs (e.g., BBMs) of the disclosure, it is advantageous to use Fab heterodimerization strategies to permit the correct association of Fab domains belonging to the same ABM and minimize aberrant pairing of Fab domains belonging to different ABMs. For example, the Fab heterodimerization strategies shown in Table 1 below can be used:

TABLE 1
Fab Heterodimerization Strategies

| Name | STRATEGY | VH | CH1 | VL | CL | REFERENCE |
|---|---|---|---|---|---|---|
| F1 | CrossMabCH1- | WT | CL domain | WT | CH1 | Schaefer et al., |
| | CL | | | | domain | 2011, Cancer Cell |
| | | | | | | 2011; 20: 472-86; |
| | | | | | | PMID: 22014573. |
| F2 | orthogonal | 39K, 62E | H172A, | 1R, 38D, | L135Y, | Lewis et al., 2014, |
| | Fab | | F174G | (36F) | S176W | Nat Biotechnol |
| | VHVRD1CH1CRD2 - | | | | | 32: 191-8 |
| | VLVRD1CλCRD2 | | | | | |
| F3 | orthogonal | 39Y | WT | 38R | WT | Lewis et al., 2014, |
| | Fab | | | | | Nat Biotechnol |
| | VHVRD2CH1wt - | | | | | 32: 191-8 |
| | VLVRD2Cλwt | | | | | |
| F4 | TCR CαCβ | 39K | TCR Cα | 38D | TCR Cβ | Wu et al., 2015, |
| | | | | | | MAbs 7: 364-76 |
| F5 | CR3 | WT | T192E | WT | N137K, | Golay at al., 2016, J |
| | | | | | S114A | Immunol 196: 3199- |
| | | | | | | 211. |
| F6 | MUT4 | WT | L143Q, | WT | V133T, | Golay at al., 2016, J |
| | | | S188V | | S176V | Immunol 196: 3199- |
| | | | | | | 211. |
| F7 | DuetMab | WT | F126C | WT | S121C | Mazor et al., 2015, |
| | | | | | | MAbs 7: 377-89; |
| | | | | | | Mazor et al., 2015, |
| | | | | | | MAbs 7: 461-669. |

Accordingly, in certain embodiments, correct association between the two polypeptides of a Fab is promoted by exchanging the VL and VH domains of the Fab for each other or exchanging the CH1 and CL domains for each other, e.g., as described in WO 2009/080251.

Correct Fab pairing can also be promoted by introducing one or more amino acid modifications in the CH1 domain and one or more amino acid modifications in the CL domain of the Fab and/or one or more amino acid modifications in the VH domain and one or more amino acid modifications in the VL domain. The amino acids that are modified are typically part of the VH:VL and CH1:CL interface such that the Fab components preferentially pair with each other rather than with components of other Fabs.

In one embodiment, the one or amino acid modifications are limited to the conserved framework residues of the variable (VH, VL) and constant (CH1, CL) domains as indicated by the Kabat numbering of residues. Almagro, 2008, Frontiers In Bioscience 13:1619-1633 provides a definition of the framework residues on the basis of Kabat, Chothia, and IMGT numbering schemes.

In one embodiment, the modifications introduced in the VH and CH1 and/or VL and CL domains are complementary to each other. Complementarity at the heavy and light chain interface can be achieved on the basis of steric and hydrophobic contacts, electrostatic/charge interactions or any combination of the variety of interactions. The complementarity between protein surfaces is broadly described in the literature in terms of lock and key fit, knob into hole, protrusion and cavity, donor and acceptor etc., all implying the nature of structural and chemical match between the two interacting surfaces.

In one embodiment, the one or more introduced modifications introduce a new hydrogen bond across the interface of the Fab components. In one embodiment, the one or more introduced modifications introduce a new salt bridge across the interface of the Fab components. Exemplary substitutions are described in WO 2014/150973 and WO 2014/082179.

In some embodiments, the Fab domain comprises a 192E substitution in the CH1 domain and 114A and 137K substitutions in the CL domain, which introduces a salt-bridge between the CH1 and CL domains (see, Golay et al., 2016, J Immunol 196:3199-211).

In some embodiments, the Fab domain comprises a 143Q and 188V substitutions in the CH1 domain and 113T and 176V substitutions in the CL domain, which serves to swap hydrophobic and polar regions of contact between the CH1 and CL domain (see, Golay et al., 2016, J Immunol 196:3199-211).

In some embodiments, the Fab domain can comprise modifications in some or all of the VH, CH1, VL, CL domains to introduce orthogonal Fab interfaces which promote correct assembly of Fab domains (Lewis et al., 2014 Nature Biotechnology 32:191-198). In an embodiment, 39K, 62E modifications are introduced in the VH domain, H172A, F174G modifications are introduced in the CH1 domain, 1R, 38D, (36F) modifications are introduced in the VL domain, and L135Y, S176W modifications are introduced in the CL domain. In another embodiment, a 39Y modification is introduced in the VH domain and a 38R modification is introduced in the VL domain.

Fab domains can also be modified to replace the native CH1:CL disulfide bond with an engineered disulfide bond, thereby increasing the efficiency of Fab component pairing. For example, an engineered disulfide bond can be introduced by introducing a 126C in the CH1 domain and a 121C in the CL domain (see, Mazor et al., 2015, MAbs 7:377-89).

Fab domains can also be modified by replacing the CH1 domain and CL domain with alternative domains that promote correct assembly. For example, Wu et al., 2015, MAbs 7:364-76, describes substituting the CH1 domain with the constant domain of the α T cell receptor and substituting the CL domain with the β domain of the T cell receptor, and pairing these domain replacements with an additional charge-charge interaction between the VL and VH domains by introducing a 38D modification in the VL domain and a 39K modification in the VH domain.

ABMs can comprise a single chain Fab fragment, which is a polypeptide consisting of an antibody heavy chain variable domain (VH), an antibody constant domain 1 (CH1), an antibody light chain variable domain (VL), an antibody light chain constant domain (CL) and a linker. In some embodiments, the antibody domains and the linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL. The linker can be a polypeptide of at least 30 amino acids, e.g., between 32 and 50 amino acids. The single chain Fab domains are stabilized via the natural disulfide bond between the CL domain and the CH1 domain.

In an embodiment, the antibody domains and the linker in the single chain Fab fragment have one of the following orders in N-terminal to C-terminal direction: a) VH-CH1-linker-VL-CL, or b) VL-CL-linker-VH-CH1. In some cases, VL-CL-linker-VH-CH1 is used.

In another embodiment, the antibody domains and the linker in the single chain Fab fragment have one of the following orders in N-terminal to C-terminal direction: a) VH-CL-linker-VL-CH1 or b) VL-CH1-linker-VH-CL.

Optionally in the single chain Fab fragment, additionally to the natural disulfide bond between the CL-domain and the CH1 domain, also the antibody heavy chain variable domain (VH) and the antibody light chain variable domain (VL) are disulfide stabilized by introduction of a disulfide bond between the following positions: i) heavy chain variable domain position 44 to light chain variable domain position 100, ii) heavy chain variable domain position 105 to light chain variable domain position 43, or iii) heavy chain variable domain position 101 to light chain variable domain position 100 (numbering according to EU index of Kabat).

Such further disulfide stabilization of single chain Fab fragments is achieved by the introduction of a disulfide bond between the variable domains VH and VL of the single chain Fab fragments. Techniques to introduce unnatural disulfide bridges for stabilization for a single chain Fv are described e.g. in WO 94/029350, Rajagopal et al., 1997, Prot. Engin. 10:1453-59; Kobayashi et al., 1998, Nuclear Medicine & Biology, 25:387-393; and Schmidt, et al., 1999, Oncogene 18:1711-1721. In one embodiment, the optional disulfide bond between the variable domains of the single chain Fab fragments is between heavy chain variable domain position 44 and light chain variable domain position 100. In one embodiment, the optional disulfide bond between the variable domains of the single chain Fab fragments is between heavy chain variable domain position 105 and light chain variable domain position 43 (numbering according to EU index of Kabat).

### 7.2.1.2. scFvs

Single chain Fv or “scFv” antibody fragments comprise the VH and VL domains of an antibody in a single polypeptide chain, are capable of being expressed as a single chain polypeptide, and retain the specificity of the intact antibody from which it is derived. Generally, the scFv polypeptide further comprises a polypeptide linker between the VH and VL domain that enables the scFv to form the desired structure for target binding. Examples of linkers suitable for connecting the VH and VL chains of an scFV are the ABM linkers identified in Section 7.3.3, for example any of the linkers designated L1 through L54.

Unless specified, as used herein an scFv can have the VL and VH variable regions in either order, e.g., with respect to the N-terminal and C-terminal ends of the polypeptide, the scFv can comprise VL-linker-VH or can comprise VH-linker-VL.

To create an scFv-encoding nucleic acid, the VH and VL-encoding DNA fragments are operably linked to another fragment encoding a linker, e.g., encoding any of the ABM linkers described in Section 7.3.3 (such as the amino acid sequence (Gly4″Ser)3 (SEQ ID NO: 53)), such that the VH and VL sequences can be expressed as a contiguous single-chain protein, with the VL and VH regions joined by the flexible linker (see e.g., Bird et al., 1988, Science 242:423-426; Huston et al., 1988, Proc. Natl. Acad. Sci. USA 85:5879-5883; McCafferty et al., 1990, Nature 348:552-554).

### 7.2.1.3. Other Immunoglobulin-Based Modules

MBMs can also comprise ABMs having an immunoglobulin format which is other than Fab or scFv, for example Fv, dsFv, (Fab′)2, a single domain antibody (SDAB), a VH or VL domain, or a camelid VHH domain (also called a nanobody).

An ABM can be a single domain antibody composed of a single VH or VL domain which exhibits sufficient affinity to the target. In some embodiments, the single domain antibody is a camelid VHH domain (see, e.g., Riechmann, 1999, Journal of Immunological Methods 231:25-38; WO 94/04678).

### 7.2.2. Non-Immunoglobulin Based Modules

In certain embodiments, one or more of the ABMs of a MBM are derived from non-antibody scaffold proteins (including, but not limited to, designed ankyrin repeat proteins (DARPins), Avimers (short for avidity multimers), Anticalin/Lipocalins, Centyrins, Kunitz domains, Adnexins, Affilins, Affitins (also known as Nonfitins), Knottins, Pronectins, Versabodies, Duocalins, and Fynomers), ligands, receptors, cytokines or chemokines.

Non-immunoglobulin scaffolds that can be used in the MBMs include those listed in Tables 3 and 4 of Mintz and Crea, 2013, Bioprocess International 11(2):40-48; in FIG. 1, Table 1 and Figure I of Vazquez-Lombardi et al., 2015, Drug Discovery Today 20(10):1271-83; in Table 1 and Box 2 of Skrlec et al., 2015, Trends in Biotechnology 33(7):408-18. The contents of Tables 3 and 4 of Mintz and Crea, 2013, Bioprocess International 11(2):40-48; in FIG. 1, Table 1 and Figure I of Vazquez-Lombardi et al., 2015, Drug Discovery Today 20(10):1271-83; in Table 1 and Box 2 of Skrlec et al., 2015, Trends in Biotechnology 33(7):408-18 (collectively, “Scaffold Disclosures”). In a particular embodiment, the Scaffold Disclosures are incorporated by reference for what they disclose relating to Adnexins. In another embodiment, the Scaffold Disclosures are incorporated by reference for what they disclose relating to Avimers. In another embodiment, the Scaffold Disclosures are incorporated by reference for what they disclose relating to Affibodies. In yet another embodiment, the Scaffold Disclosures are incorporated by reference for what they disclose relating to Anticalins. In yet another embodiment, the Scaffold Disclosures are incorporated by reference for what they disclose relating to DARPins. In yet another embodiment, the Scaffold Disclosures are incorporated by reference for what they disclose relating to Kunitz domains. In yet another embodiment, the Scaffold Disclosures are incorporated by reference for what they disclose relating to Knottins. In yet another embodiment, the Scaffold Disclosures are incorporated by reference for what they disclose relating to Pronectins. In yet another embodiment, the Scaffold Disclosures are incorporated by reference for what they disclose relating to Nanofitins. In yet another embodiment, the Scaffold Disclosures are incorporated by reference for what they disclose relating to Affilins. In yet another embodiment, the Scaffold Disclosures are incorporated by reference for what they disclose relating to Adnectins. In yet another embodiment, the Scaffold Disclosures are incorporated by reference for what they disclose relating to ABDs. In yet another embodiment, the Scaffold Disclosures are incorporated by reference for what they disclose relating to Adhirons. In yet another embodiment, the Scaffold Disclosures are incorporated by reference for what they disclose relating to Affimers. In yet another embodiment, the Scaffold Disclosures are incorporated by reference for what they disclose relating to Alphabodies. In yet another embodiment, the Scaffold Disclosures are incorporated by reference for what they disclose relating to Armadillo Repeat Proteins. In yet another embodiment, the Scaffold Disclosures are incorporated by reference for what they disclose relating to Atrimers/Tetranectins. In yet another embodiment, the Scaffold Disclosures are incorporated by reference for what they disclose relating to Obodies/OB-folds. In yet another embodiment, the Scaffold Disclosures are incorporated by reference for what they disclose relating to Centyrins. In yet another embodiment, the Scaffold Disclosures are incorporated by reference for what they disclose relating to Repebodies. In yet another embodiment, the Scaffold Disclosures are incorporated by reference for what they disclose relating to Anticalins. In yet another embodiment, the Scaffold Disclosures are incorporated by reference for what they disclose relating to Atrimers. In yet another embodiment, the Scaffold Disclosures are incorporated by reference for what they disclose relating to bicyclic peptides. In yet another embodiment, the Scaffold Disclosures are incorporated by reference for what they disclose relating to cys-knots. In yet another embodiment, the Scaffold Disclosures are incorporated by reference for what they disclose relating to Fn3 scaffolds (including Adnectins, Centryrins, Pronectins, and Tn3).

In an embodiment, an ABM can be a designed ankyrin repeat protein (“DARPin”). DARPins are antibody mimetic proteins that typically exhibit highly specific and high-affinity target protein binding. They are typically genetically engineered and derived from natural ankyrin proteins and consist of at least three, usually four or five repeat motifs of these proteins. Their molecular mass is about 14 or 18 kDa (kilodaltons) for four- or five-repeat DARPins, respectively. Examples of DARPins can be found, for example in U.S. Pat. No. 7,417,130. Multispecific binding molecules comprising DARPin binding modules and immunoglobulin-based binding modules are disclosed in, for example, U.S. Publication No. 2015/0030596 A1.

In another embodiment, an ABM can be an Affibody. An Affibody is well known and refers to affinity proteins based on a 58 amino acid residue protein domain, derived from one of the IgG binding domain of staphylococcal protein A.

In another embodiment, an ABM can be an Anticalin. Anticalins are well known and refer to another antibody mimetic technology, where the binding specificity is derived from Lipocalins. Anticalins can also be formatted as dual targeting protein, called Duocalins.

In another embodiment, an ABM can be a Versabody. Versabodies are well known and refer to another antibody mimetic technology. They are small proteins of 3-5 kDa with >15% cysteines, which form a high disulfide density scaffold, replacing the hydrophobic core of typical proteins.

Other non-immunoglobulin ABMs include “A” domain oligomers (also known as Avimers) (see for example, U.S. Patent Application Publication Nos. 2005/0164301, 2005/0048512, and 2004/017576), Fn3 based protein scaffolds (see for example, U.S. Patent Application Publication 2003/0170753), VASP polypeptides, Avian pancreatic polypeptide (aPP), Tetranectin (based on CTLD3), Affililin (based on γB-crystallin/ubiquitin), Knottins, SH3 domains, PDZ domains, Tendamistat, Neocarzinostatin, Protein A domains, Lipocalins, Transferrin, or Kunitz domains. In one aspect, ABMs useful in the construction of the MBMs comprise fibronectin-based scaffolds as exemplified in WO 2011/130324.

### 7.3. Connectors

It is contemplated that the MBMs can in some instances include pairs of ABMs or ABM chains (e.g., the VH-CH1 or VL-CL component of a Fab) connected directly to one another, e.g., as a fusion protein without a linker. For example, the MBMs comprise connector moieties linking individual ABMs or ABM chains. The use of connector moieties can improve target binding, for example by increasing flexibility of the ABMs within a MBM and thus reducing steric hindrance. The ABMs can be connected to one another through, for example, Fc domains (each Fc domain representing a pair of associated Fc regions) and/or ABM linkers. The use of Fc domains will typically require the use of hinge regions as connectors of the ABMs or ABM chains for optimal antigen binding. Thus, the term “connector” encompasses, but is not limited to, Fc regions, Fc domains, hinge regions, and ABM linkers.

Connectors can be selected or modified to, for example, increase or decrease the biological half-life of a MBM of the disclosure. For example, to decrease biological half-life, one or more amino acid mutations can be introduced into a CH2-CH3 domain interface region of an Fc-hinge fragment such that a MBM comprising the fragment has impaired Staphylococcyl Protein A (SpA) binding relative to native Fc-hinge domain SpA binding. This approach is described in further detail in U.S. Pat. No. 6,165,745 by Ward et al. Alternatively, a MBM can be modified to increase its biological half-life. For example, one or more of the following mutations can be introduced: T252L, T254S, T256F, as described in U.S. Pat. No. 6,277,375 to Ward. Alternatively, to increase the biological half-life, a MBM can be altered within a CH1 or CL region to contain a salvage receptor binding epitope taken from two loops of a CH2 domain of an Fc region of an IgG, as described in U.S. Pat. Nos. 5,869,046 and 6,121,022 by Presta et al.

Examples of Fc domains (formed by the pairing of two Fc regions), hinge regions and ABM linkers are described in Sections 7.3.1, 7.3.2, and 7.3.3, respectively.

### 7.3.1. Fc Domains

The MBMs (e.g., BBMs) can include an Fc domain derived from any suitable species. In one embodiment, the Fc domain is derived from a human Fc domain.

The Fc domain can be derived from any suitable class of antibody, including IgA (including subclasses IgA1 and IgA2), IgD, IgE, IgG (including subclasses IgG1, IgG2, IgG3 and IgG4), and IgM. In one embodiment, the Fc domain is derived from IgG1, IgG2, IgG3 or IgG4. In one embodiment, the Fc domain is derived from IgG1. In one embodiment, the Fc domain is derived from IgG4.

The Fc domain comprises two polypeptide chains, each referred to as a heavy chain Fc region. The two heavy chain Fc regions dimerize to create the Fc domain. The two Fc regions within the Fc domain can be the same or different from one another. In a native antibody the Fc regions are typically identical, but for the purpose of producing multispecific binding molecules, e.g., the BBMs of the disclosure, the Fc regions might advantageously be different to allow for heterodimerization, as described in Section 7.3.1.5 below.

Typically each heavy chain Fc region comprises or consists of two or three heavy chain constant domains.

In native antibodies, the heavy chain Fc region of IgA, IgD and IgG is composed of two heavy chain constant domains (CH2 and CH3) and that of IgE and IgM is composed of three heavy chain constant domains (CH2, CH3 and CH4). These dimerize to create an Fc domain.

In the present disclosure, the heavy chain Fc region can comprise heavy chain constant domains from one or more different classes of antibody, for example one, two or three different classes.

In one embodiment, the heavy chain Fc region comprises CH2 and CH3 domains derived from IgG1.

In one embodiment, the heavy chain Fc region comprises CH2 and CH3 domains derived from IgG2.

In one embodiment, the heavy chain Fc region comprises CH2 and CH3 domains derived from IgG3.

In one embodiment, the heavy chain Fc region comprises CH2 and CH3 domains derived from IgG4.

In one embodiment, the heavy chain Fc region comprises a CH4 domain from IgM. The IgM CH4 domain is typically located at the C-terminus of the CH3 domain.

In one embodiment, the heavy chain Fc region comprises CH2 and CH3 domains derived from IgG and a CH4 domain derived from IgM.

It will be appreciated that the heavy chain constant domains for use in producing a heavy chain Fc region for the MBMs of the present disclosure can include variants of the naturally occurring constant domains described above. Such variants can comprise one or more amino acid variations compared to wild type constant domains. In one example the heavy chain Fc region of the present disclosure comprises at least one constant domain that varies in sequence from the wild type constant domain. It will be appreciated that the variant constant domains can be longer or shorter than the wild type constant domain. For example, the variant constant domains are at least 60% identical or similar to a wild type constant domain. In another example the variant constant domains are at least 70% identical or similar. In another example the variant constant domains are at least 75% identical or similar. In another example the variant constant domains are at least 80% identical or similar. In another example the variant constant domains are at least 85% identical or similar. In another example the variant constant domains are at least 90% identical or similar. In another example the variant constant domains are at least 95% identical or similar. In another example the variant constant domains are at least 99% identical or similar. Exemplary Fc variants are described in Sections 7.3.1.1 through 7.3.1.5, infra.

IgM and IgA occur naturally in humans as covalent multimers of the common H2L2 antibody unit. IgM occurs as a pentamer when it has incorporated a J-chain, or as a hexamer when it lacks a J-chain. IgA occurs as monomer and dimer forms. The heavy chains of IgM and IgA possess an 18 amino acid extension to the C-terminal constant domain, known as a tailpiece. The tailpiece includes a cysteine residue that forms a disulfide bond between heavy chains in the polymer, and is believed to have an important role in polymerization. The tailpiece also contains a glycosylation site. In certain embodiments, the MBMs of the present disclosure do not comprise a tailpiece.

The Fc domains that are incorporated into the MBMs (e.g., BBMs) of the present disclosure can comprise one or more modifications that alter one or more functional properties of the proteins, such as serum half-life, complement fixation, Fc receptor binding, and/or antigen-dependent cellular cytotoxicity. Furthermore, a MBM can be chemically modified (e.g., one or more chemical moieties can be attached to the MBM) or be modified to alter its glycosylation, again to alter one or more functional properties of the MBM.

Effector function of an antibody molecule includes complement-mediated effector function, which is mediated by, for example, binding of the C1 component of the complement to the antibody. Activation of complement is important in the opsonization and direct lysis of pathogens. In addition, it stimulates the inflammatory response by recruiting and activating phagocytes to the site of complement activation. Effector function includes Fc receptor (FcR)-mediated effector function, which can be triggered upon binding of the constant domains of an antibody to an Fc receptor (FcR). Antigen-antibody complex-mediated crosslinking of Fc receptors on effector cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production.

Fc regions can be altered by replacing at least one amino acid residue with a different amino acid residue to alter the effector functions. For example, one or more amino acids can be replaced with a different amino acid residue such that the Fc region has an altered affinity for an effector ligand. The effector ligand to which affinity is altered can be, for example, an Fc receptor or the C1 component of complement. This approach is described in, e.g., U.S. Pat. Nos. 5,624,821 and 5,648,260, both by Winter et al. Modified Fc regions can also alter C1q binding and/or reduce or abolish complement dependent cytotoxicity (CDC). This approach is described in, e.g., U.S. Pat. No. 6,194,551 by Idusogie et al. Modified Fc regions can also alter the ability of an Fc region to fix complement. This approach is described in, e.g., the PCT Publication WO 94/29351 by Bodmer et al. Allotypic amino acid residues include, but are not limited to, constant region of a heavy chain of the IgG1, IgG2, and IgG3 subclasses as well as constant region of a light chain of the kappa isotype as described by Jefferis et al., 2009, MAbs, 1:332-338.

Fc regions can also be modified to “silence” the effector function, for example, to reduce or eliminate the ability of a MBM to mediate antibody dependent cellular cytotoxicity (ADCC) and/or antibody dependent cellular phagocytosis (ADCP). This can be achieved, for example, by introducing a mutation in an Fc region. Such mutations have been described in the art: LALA and N297A (Strohl, 2009, Curr. Opin. Biotechnol. 20(6):685-691); and D265A (Baudino et al., 2008, J. Immunol. 181: 6664-69; Strohl, supra). Examples of silent Fc IgG1 antibodies comprise the so-called LALA mutant comprising L234A and L235A mutation in the IgG1 Fc amino acid sequence. Another example of a silent IgG1 antibody comprises the D265A mutation. Another silent IgG1 antibody comprises the so-called DAPA mutant comprising D265A and P329A mutations in the IgG1 Fc amino acid sequence. Another silent IgG1 antibody comprises the N297A mutation, which results in aglycosylated/non-glycosylated antibodies.

Fc regions can be modified to increase the ability of a MBM containing the Fc region to mediate antibody dependent cellular cytotoxicity (ADCC) and/or antibody dependent cellular phagocytosis (ADCP), for example, by modifying one or more amino acid residues to increase the affinity of the MBM for an activating Fcγ receptor, or to decrease the affinity of the MBM for an inhibitory Fcγ receptor. Human activating Fcγ receptors include FcγRIa, FcγRIIa, FcγRIIIa, and FcγRIIIb, and human inhibitory Fcγ receptor includes FcγRIIb. This approach is described in, e.g., the PCT Publication WO 00/42072 by Presta. Moreover, binding sites on human IgG1 for FcγRI, FcγRII, FcγRIII and FcRn have been mapped and variants with improved binding have been described (see Shields et al., J. Biol. Chem. 276:6591-6604, 2001). Optimization of Fc-mediated effector functions of monoclonal antibodies such as increased ADCC/ADCP function has been described (see Strohl, 2009, Current Opinion in Biotechnology 20:685-691). Mutations that can enhance ADCC/ADCP function include one or more mutations selected from G236A, S239D, F243L, P2471, D280H, K290S, R292P, S298A, S298D, S298V, Y300L, V305I, A330L, I332E, E333A, K334A, A339D, A339Q, A339T, and P396L (all positions by EU numbering).

Fc regions can also be modified to increase the ability of a MBM to mediate ADCC and/or ADCP, for example, by modifying one or more amino acids to increase the affinity of the MBM for an activating receptor that would typically not recognize the parent MBM, such as FcαRI. This approach is described in, e.g., Borrok et al., 2015, mAbs. 7(4):743-751.

Accordingly, in certain aspects, the MBMs of the present disclosure can include Fc domains with altered effector function such as, but not limited, binding to Fc-receptors such as FcRn or leukocyte receptors (for example, as described above or in Section 7.3.1.1), binding to complement (for example as described above or in Section 7.3.1.2), modified disulfide bond architecture (for example as described above or in Section 7.3.1.3), or altered glycosylation patterns (for example as described above or in Section 7.3.1.4). The Fc domains can also be altered to include modifications that improve manufacturability of asymmetric MBMs, for example by allowing heterodimerization, which is the preferential pairing of non-identical Fc regions over identical Fc regions. Heterodimerization permits the production of MBMs in which different ABMs are connected to one another by an Fc domain containing Fc regions that differ in sequence. Examples of heterodimerization strategies are exemplified in Section 7.3.1.5 (and subsections thereof).

It will be appreciated that any of the modifications described in Sections 7.3.1.1 through 7.3.1.5 can be combined in any suitable manner to achieve the desired functional properties and/or combined with other modifications to alter the properties of the MBMs.

### 7.3.1.1. Fc Domains with Altered FcR Binding

The Fc domains of the MBMs (e.g., BBMs) can show altered binding to one or more Fc-receptors (FcRs) in comparison with the corresponding native immunoglobulin. The binding to any particular Fc-receptor can be increased or decreased. In one embodiment, the Fc domain comprises one or more modifications which alter its Fc-receptor binding profile.

Human cells can express a number of membrane bound FcRs selected from FcαR, FcεR, FcγR, FcRn and glycan receptors. Some cells are also capable of expressing soluble (ectodomain) FcR (Fridman et al., 1993, J Leukocyte Biology 54: 504-512). FcγR can be further divided by affinity of IgG binding (high/low) and biological effect (activating/inhibiting). Human FcγRI is widely considered to be the sole ‘high affinity’ receptor whilst all of the others are considered as medium to low. FcγRIIb is the sole receptor with ‘inhibitory’ functionality by virtue of its intracellular ITIM motif whilst all of the others are considered as ‘activating’ by virtue of ITAM motifs or pairing with the common FcγR-γchain. FcγRIIIb is also unique in that although activatory it associates with the cell via a GPI anchor. In total, humans express six “standard” FcγRs: FcγRI, FcγRIIa, FcγRIIb, FcγRIIc, FcγRIIIa, and FcγRIIIb. In addition to these sequences there are a large number of sequence or allotypic variants spread across these families. Some of these have been found to have important functional consequence and so are sometimes considered to be receptor sub-types of their own. Examples include FcγRIIa^{H134R}, FcγRIIb^{I190T}, FcγRIIIa^{F158V}, FcγRIIIb^{NA1}, FcγRIII^{NA2}, and FcγRIII^{SH}. Each receptor sequence has been shown to have different affinities for the 4 sub-classes of IgG: IgG1, IgG2, IgG3 and IgG4 (Bruhns, 1993, Blood 113:3716-3725). Other species have somewhat different numbers and functionality of FcγR, with the mouse system being the best studied to date and comprising of 4 FcγR, FcγRI FcγRIIb FcγRIII FcγRIV (Bruhns, 2012, Blood 119:5640-5649). Human FcγRI on cells is normally considered to be ‘occupied’ by monomeric IgG in normal serum conditions due to its affinity for IgG1/IgG3/IgG4 (about 10⁻⁸ M) and the concentration of these IgG in serum (about 10 mg/ml). Hence cells bearing FcγRI on their surface are considered to be capable for “screening” or “sampling” of their antigenic environment vicariously through the bound polyspecific IgG. The other receptors having lower affinities for IgG sub-classes (in the range of about 10⁻⁵-10⁻⁷ M) are normally considered to be “unoccupied.” The low affinity receptors are hence inherently sensitive to the detection of and activation by antibody involved immune complexes. The increased Fc density in an antibody immune complex results in increased functional affinity of binding avidity to low affinity FcγR. This has been demonstrated in vitro using a number of methods (Shields et al., 2001, J Biol Chem 276(9):6591-6604; Lux et al., 2013, J Immunol 190:4315-4323). It has also been implicated as being one of the primary modes of action in the use of anti-RhD to treat ITP in humans (Crow, 2008, Transfusion Medicine Reviews 22:103-116).

Many cell types express multiple types of FcγR and so binding of IgG or antibody immune complex to cells bearing FcγR can have multiple and complex outcomes depending upon the biological context. Most simply, cells can either receive an activatory, inhibitory or mixed signal. This can result in events such as phagocytosis (e.g., macrophages and neutrophils), antigen processing (e.g., dendritic cells), reduced IgG production (e.g., B-cells) or degranulation (e.g., neutrophils, mast cells). There are data to support that the inhibitory signal from FcγRIIb can dominate that of activatory signals (Proulx, 2010, Clinical Immunology 135:422-429).

There are a number of useful Fc substitutions that can be made to alter binding to one or more of the FcγR receptors. Substitutions that result in increased binding as well as decreased binding can be useful. For example, it is known that increased binding to FcγRIIIa generally results in increased ADCC (antibody dependent cell-mediated cytotoxicity; the cell-mediated reaction where nonspecific cytotoxic cells that express FcγRs recognize bound antibody on a target cell and subsequently cause lysis of the target cell). Similarly, decreased binding to FcγRIIb (an inhibitory receptor) can be beneficial as well in some circumstances. Amino acid substitutions that find use in the present disclosure include those listed in US 2006/0024298 (particularly FIG. 41), US 2006/0121032, US 2006/0235208, and US 2007/0148170. Particular variants that find use include, but are not limited to, 236A, 239D, 239E, 332E, 332D, 239D/332E, 267D, 267E, 328F, 267E/328F, 236A/332E, 239D/332E/330Y, 239D, 332E/330L, 243A, 243L, 264A, 264V and 299T.

FcRn has a crucial role in maintaining the long half-life of IgG in the serum of adults and children. The receptor binds IgG in acidified vesicles (pH<6.5) protecting the IgG molecule from degradation, and then releasing it at the higher pH of 7.4 in blood.

FcRn is unlike leukocyte Fc receptors, and instead, has structural similarity to MHC class I molecules. It is a heterodimer composed of a β₂-microglobulin chain, non-covalently attached to a membrane-bound chain that includes three extracellular domains. One of these domains, including a carbohydrate chain, together with β₂-microglobulin interacts with a site between the CH2 and CH3 domains of Fc. The interaction includes salt bridges made to histidine residues on IgG that are positively charged at pH<6.5. At higher pH, the His residues lose their positive charges, the FcRn-IgG interaction is weakened and IgG dissociates.

In one embodiment, a MBM comprises an Fc domain that binds to human FcRn.

In one embodiment, the Fc domain has an (e.g., one or two) Fc regions comprising a histidine residue at position 310, and in some cases also at position 435. These histidine residues are important for human FcRn binding. In one embodiment, the histidine residues at positions 310 and 435 are native residues, i.e., positions 310 and 435 are not modified. Alternatively, one or both of these histidine residues can be present as a result of a modification.

The MBMs can comprise one or more Fc regions that alter Fc binding to FcRn. The altered binding can be increased binding or decreased binding.

In one embodiment, the MBM comprises an Fc domain in which at least one (and optionally both) Fc regions comprises one or more modifications such that it binds to FcRn with greater affinity and avidity than the corresponding native immunoglobulin.

Fc substitutions that increase binding to the FcRn receptor and increase serum half life are described in US 2009/0163699, including, but not limited to, 434S, 434A, 428L, 308F, 259I, 428L/434S, 259I/308F, 436I/428L, 436I or V/434S, 436V/428L and 259I/308F/428L.

In one embodiment, the Fc region is modified by substituting the threonine residue at position 250 with a glutamine residue (T250Q).

In one embodiment, the Fc region is modified by substituting the methionine residue at position 252 with a tyrosine residue (M252Y)

In one embodiment, the Fc region is modified by substituting the serine residue at position 254 with a threonine residue (S254T).

In one embodiment, the Fc region is modified by substituting the threonine residue at position 256 with a glutamic acid residue (T256E).

In one embodiment, the Fc region is modified by substituting the threonine residue at position 307 with an alanine residue (T307A).

In one embodiment, the Fc region is modified by substituting the threonine residue at position 307 with a proline residue (T307P).

In one embodiment, the Fc region is modified by substituting the valine residue at position 308 with a cysteine residue (V308C).

In one embodiment, the Fc region is modified by substituting the valine residue at position 308 with a phenylalanine residue (V308F).

In one embodiment, the Fc region is modified by substituting the valine residue at position 308 with a proline residue (V308P).

In one embodiment, the Fc region is modified by substituting the glutamine residue at position 311 with an alanine residue (Q311A).

In one embodiment, the Fc region is modified by substituting the glutamine residue at position 311 with an arginine residue (Q311R).

In one embodiment, the Fc region is modified by substituting the methionine residue at position 428 with a leucine residue (M428L).

In one embodiment, the Fc region is modified by substituting the histidine residue at position 433 with a lysine residue (H433K).

In one embodiment, the Fc region is modified by substituting the asparagine residue at position 434 with a phenylalanine residue (N434F).

In one embodiment, the Fc region is modified by substituting the asparagine residue at position 434 with a tyrosine residue (N434Y).

In one embodiment, the Fc region is modified by substituting the methionine residue at position 252 with a tyrosine residue, the serine residue at position 254 with a threonine residue, and the threonine residue at position 256 with a glutamic acid residue (M252Y/S254T/T256E).

In one embodiment, the Fc region is modified by substituting the valine residue at position 308 with a proline residue and the asparagine residue at position 434 with a tyrosine residue (V308P/N434Y).

In one embodiment, the Fc region is modified by substituting the methionine residue at position 252 with a tyrosine residue, the serine residue at position 254 with a threonine residue, the threonine residue at position 256 with a glutamic acid residue, the histidine residue at position 433 with a lysine residue and the asparagine residue at position 434 with a phenylalanine residue (M252Y/S254T/T256E/H433K/N434F).

It will be appreciated that any of the modifications listed above can be combined to alter FcRn binding.

In one embodiment, the MBM comprises an Fc domain in which one or both Fc regions comprise one or more modifications such that the Fc domain binds to FcRn with lower affinity and avidity than the corresponding native immunoglobulin.

In one embodiment, the Fc region comprises any amino acid residue other than histidine at position 310 and/or position 435.

The MBM can comprise an Fc domain in which one or both Fc regions comprise one or more modifications which increase its binding to FcγRIIb. FcγRIIb is the only inhibitory receptor in humans and the only Fc receptor found on B cells.

In one embodiment, the Fc region is modified by substituting the proline residue at position 238 with an aspartic acid residue (P238D).

In one embodiment, the Fc region is modified by substituting the glutamic acid residue at position 258 with an alanine residue (E258A).

In one embodiment, the Fc region is modified by substituting the serine residue at position 267 with an alanine residue (S267A).

In one embodiment, the Fc region is modified by substituting the serine residue at position 267 with a glutamic acid residue (S267E).

In one embodiment, the Fc region is modified by substituting the leucine residue at position 328 with a phenylalanine residue (L328F).

In one embodiment, the Fc region is modified by substituting the glutamic acid residue at position 258 with an alanine residue and the serine residue at position 267 with an alanine residue (E258A/S267A).

In one embodiment, the Fc region is modified by substituting the serine residue at position 267 with a glutamic acid residue and the leucine residue at position 328 with a phenylalanine residue (S267E/L328F).

It will be appreciated that any of the modifications listed above can be combined to increase FcγRIIb binding.

In one embodiment, MBMs are provided comprising Fc domains which display decreased binding to FcγR.

In one embodiment, an MBM comprises an Fc domain in which one or both Fc regions comprise one or more modifications that decrease Fc binding to FcγR.

The Fc domain can be derived from IgG1.

In one embodiment, the Fc region is modified by substituting the leucine residue at position 234 with an alanine residue (L234A).

In one embodiment, the Fc region is modified by substituting the leucine residue at position 235 with an alanine residue (L235A).

In one embodiment, the Fc region is modified by substituting the glycine residue at position 236 with an arginine residue (G236R).

In one embodiment, the Fc region is modified by substituting the asparagine residue at position 297 with an alanine residue (N297A) or a glutamine residue (N297Q).

In one embodiment, the Fc region is modified by substituting the serine residue at position 298 with an alanine residue (S298A).

In one embodiment, the Fc region is modified by substituting the leucine residue at position 328 with an arginine residue (L328R).

In one embodiment, the Fc region is modified by substituting the leucine residue at position 234 with an alanine residue and the leucine residue at position 235 with an alanine residue (L234A/L235A).

In one embodiment, the Fc region is modified by substituting the phenylalanine residue at position 234 with an alanine residue and the leucine residue at position 235 with an alanine residue (F234A/L235A).

In one embodiment, the Fc region is modified by substituting the glycine residue at position 236 with an arginine residue and the leucine residue at position 328 with an arginine residue (G236R/L328R).

It will be appreciated that any of the modifications listed above can be combined to decrease FcγR binding.

In one embodiment, a MBM comprises an Fc domain in which one or both Fc regions comprise one or more modifications that decrease Fc binding to FcγRIIIa without affecting the Fc's binding to FcγRII.

In one embodiment, the Fc region is modified by substituting the serine residue at position 239 with an alanine residue (S239A).

In one embodiment, the Fc region is modified by substituting the glutamic acid residue at position 269 with an alanine residue (E269A).

In one embodiment, the Fc region is modified by substituting the glutamic acid residue at position 293 with an alanine residue (E293A).

In one embodiment, the Fc region is modified by substituting the tyrosine residue at position 296 with a phenylalanine residue (Y296F).

In one embodiment, the Fc region is modified by substituting the valine residue at position 303 with an alanine residue (V303A).

In one embodiment, the Fc region is modified by substituting the alanine residue at position 327 with a glycine residue (A327G).

In one embodiment, the Fc region is modified by substituting the lysine residue at position 338 with an alanine residue (K338A).

In one embodiment, the Fc region is modified by substituting the aspartic acid residue at position 376 with an alanine residue (D376A).

It will be appreciated that any of the modifications listed above can be combined to decrease FcγRIIIa binding.

Fc region variants with decreased FcR binding can be referred to as “FcγR ablation variants,” “FcγR silencing variants” or “Fc knock out (FcKO or KO)” variants. For some therapeutic applications, it is desirable to reduce or remove the normal binding of an Fc domain to one or more or all of the Fcγ receptors (e.g., FcγR1, FcγRIIa, FcγRIIb, FcγRIIIa) to avoid additional mechanisms of action. That is, for example, in many embodiments, particularly in the use of MBMs that bind CD3 monovalently, it is generally desirable to ablate FcγRIIIa binding to eliminate or significantly reduce ADCC activity. In some embodiments, at least one of the Fc regions of the MBMs described herein comprises one or more Fcγ receptor ablation variants. In some embodiments, both of the Fc regions comprise one or more Fcγ receptor ablation variants. These ablation variants are depicted in Table 2, and each can be independently and optionally included or excluded, with some aspects utilizing ablation variants selected from the group consisting of G236R/L328R, E233P/L234V/L235A/G236del/S239K, E233P/L234V/L235A/G236del/S267K, E233P/L234V/L235A/G236del/S239K/A327G, E233P/L234V/L235A/G236del/S267K/A327G and E233P/L234V/L235A/G236del (“del” connotes a deletion, e.g., G236del refers to a deletion of the glycine at position 236). It should be noted that the ablation variants referenced herein ablate FcγR binding but generally not FcRn binding.

TABLE 2
Ablation Variants

| Variant | Variant(s), cont. |
|---|---|
| G236R | P329K |
| S239G | A330L |
| S239K | A330S/P331S |
| S239Q | I332K |
| S239R | I332R |
| V266D | V266D/A327Q |
| S267K | V266D/P329K |
| S267R | S267R/A327Q |
| H268K | S267R/P329K |
| E269R | G236R/L328R |
| 299R | E233P/L234V/L235A/G236del/S239K |
| 299K | E233P/L234V/L235A/G236del/S267K |
| K322A | E233P/L234V/L235A/G236del/S239K/A327G |
| A327G | E233P/L234V/L235A/G236del/S267K/A327G |
| A327L | E233P/L234V/L235A/G236del |
| A327N | S239K/S267K |
| A327Q | 267K/P329K |
| L328E | |
| L328R | |
| P329A | |
| P329H | |

In some embodiments, the MBMs of the present disclosure comprises a first Fc region and a second Fc region. In some embodiments, the first Fc region and/or the second Fc region can comprise the following mutations: E233P, L234V, L235A, G236del, and S267K.

The Fc domain of human IgG1 has the highest binding to the Fcγ receptors, and thus ablation variants can be used when the constant domain (or Fc domain) in the backbone of the heterodimeric antibody is IgG1.

Alternatively, or in addition to ablation variants in an IgG1 background, mutations at the glycosylation position 297, e.g., substituting the asparagine residue at position 297 with an alanine residue (N297A) or a glutamine residue (N297Q), can significantly ablate binding to FcγRIIIa, for example. Human IgG2 and IgG4 have naturally reduced binding to the Fcγ receptors, and thus those backbones can be used with or without the ablation variants.

### 7.3.1.2. Fc Domains with Altered Complement Binding

An MBM (e.g., BBM) can comprise an Fc domain in which one or both Fc regions comprises one or more modifications that alter Fc binding to complement. Altered complement binding can be increased binding or decreased binding.

In one embodiment, the Fc region comprises one or more modifications which decrease its binding to C1q. Initiation of the classical complement pathway starts with binding of hexameric C1q protein to the CH2 domain of antigen bound IgG and IgM.

In one embodiment, the MBM comprises an Fc domain in which one or both Fc regions comprises one or more modifications to decrease Fc binding to C1q.

In one embodiment, the Fc region is modified by substituting the leucine residue at position 234 with an alanine residue (L234A).

In one embodiment, the Fc region is modified by substituting the leucine residue at position 235 with an alanine residue (L235A).

In one embodiment, the Fc region is modified by substituting the leucine residue at position 235 with a glutamic acid residue (L235E).

In one embodiment, the Fc region is modified by substituting the glycine residue at position 237 with an alanine residue (G237A).

In one embodiment, the Fc region is modified by substituting the lysine residue at position 322 with an alanine residue (K322A).

In one embodiment, the Fc region is modified by substituting the proline residue at position 331 with an alanine residue (P331A).

In one embodiment, the Fc region is modified by substituting the proline residue at position 331 with a serine residue (P331S).

In one embodiment, a MBM comprises an Fc domain derived from IgG4. IgG4 has a naturally lower complement activation profile than IgG1, but also weaker binding of FcγR. Thus, in one embodiment, the MBM comprises an IgG4 Fc domain and also comprises one or more modifications that increase FcγR binding.

It will be appreciated that any of the modifications listed above can be combined to reduce C1q binding.

### 7.3.1.3. Fc Domains with Altered Disulfide Architecture

An MBM (e.g., BBM) can include an Fc domain comprising one or more modifications to create and/or remove a cysteine residue. Cysteine residues have an important role in the spontaneous assembly of Fc-based multispecific binding molecules, by forming disulfide bridges between individual pairs of polypeptide monomers. Thus, by altering the number and/or position of cysteine residues, it is possible to modify the structure of the MBM to produce a protein with improved therapeutic properties.

A MBM can comprise an Fc domain in which one or both Fc regions, e.g., both Fc regions, comprise a cysteine residue at position 309. In one embodiment, the cysteine residue at position 309 is created by a modification, e.g., for an Fc domain derived from IgG1, the leucine residue at position 309 is substituted with a cysteine residue (L309C), for an Fc domain derived from IgG2, the valine residue at position 309 is substituted with a cysteine residue (V309C).

In one embodiment, the Fc region is modified by substituting the valine residue at position 308 with a cysteine residue (V308C).

In one embodiment, two disulfide bonds in the hinge region are removed by mutating a core hinge sequence CPPC (SEQ ID NO: 55) to SPPS (SEQ ID NO: 56).

### 7.3.1.4. Fc Domains with Altered Glycosylation

In certain aspects, MBMs (e.g., BBMs) with improved manufacturability are provided that comprise fewer glycosylation sites than a corresponding immunoglobulin. These proteins have less complex post translational glycosylation patterns and are thus simpler and less expensive to manufacture.

In one embodiment, a glycosylation site in the CH2 domain is removed by substituting the asparagine residue at position 297 with an alanine residue (N297A) or a glutamine residue (N297Q). In addition to improved manufacturability, these aglycosyl mutants also reduce FcγR binding as described herein above.

In some embodiments, a MBM can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing a MBM in a host cell with altered glycosylation machinery. Cells with altered glycosylation machinery have been described in the art and can be used as host cells in which to express MBMs to thereby produce MBM with altered glycosylation. For example, EP 1,176,195 by Hang et al. describes a cell line with a functionally disrupted FUT8 gene, which encodes a fucosyl transferase, such that antibodies expressed in such a cell line exhibit hypofucosylation. PCT Publication WO 03/035835 by Presta describes a variant CHO cell line, Lecl3 cells, with reduced ability to attach fucose to Asn(297)-linked carbohydrates, also resulting in hypofucosylation of antibodies expressed in that host cell (see also Shields et al., 2002, J. Biol. Chem. 277:26733-26740). PCT Publication WO 99/54342 by Umana et al. describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures which results in increased ADCC activity of the antibodies (see also Umana et al., Nat. Biotech. 17:176-180, 1999).

### 7.3.1.5. Fc Heterodimerization

Many multispecific molecule formats entail dimerization between two Fc regions that, unlike a native immunoglobulin, are operably linked to non-identical antigen-binding domains (or portions thereof, e.g., a VH or VH-CH1 of a Fab). Inadequate heterodimerization of two Fc regions to form an Fc domain has always been an obstacle for increasing the yield of desired multispecific molecules and represents challenges for purification. A variety of approaches available in the art can be used in for enhancing dimerization of Fc regions that might be present in the MBMs (e.g., BBMs) of the disclosure, for example as disclosed in EP 1870459A1; U.S. Pat. Nos. 5,582,996; 5,731,168; 5,910,573; 5,932,448; 6,833,441; 7,183,076; U.S. Patent Application Publication No. 2006204493A1; and PCT Publication No. WO2009/089004A1.

The present disclosure provides MBMs (e.g., BBMs) comprising Fc heterodimers, i.e., Fc domains comprising heterologous, non-identical Fc regions. Heterodimerization strategies are used to enhance dimerization of Fc regions operably linked to different ABMs (or portions thereof, e.g., a VH or VH-CH1 of a Fab) and reduce dimerization of Fc regions operably linked to the same ABM or portion thereof. Typically, each Fc region in the Fc heterodimer comprises a CH3 domain of an antibody. The CH3 domains are derived from the constant region of an antibody of any isotype, class or subclass, and in some cases, of IgG (IgG1, IgG2, IgG3 and IgG4) class, as described in the preceding section.

Typically, the MBMs comprise other antibody fragments in addition to CH3 domains, such as, CH1 domains, CH2 domains, hinge domain, VH domain(s), VL domain(s), CDR(s), and/or antigen-binding fragments described herein. In some embodiments, the two hetero-polypeptides are two heavy chains forming a bispecific or multispecific molecules. Heterodimerization of the two different heavy chains at CH3 domains give rise to the desired antibody or antibody-like molecule, while homodimerization of identical heavy chains will reduce yield of the desired antibody or molecule. In an exemplary embodiment, the two or more hetero-polypeptide chains comprise two chains comprising CH3 domains and forming the molecules of any of the multispecific molecule formats described above of the present disclosure. In an embodiment, the two hetero-polypeptide chains comprising CH3 domains comprise modifications that favor heterodimeric association of the polypeptides, relative to unmodified chains. Various examples of modification strategies are provided below in Table 3 and Sections 7.3.1.5.1 to 7.3.1.5.7.

TABLE 3
Fc Heterodimerization Strategies

| NO. | STRATEGY | CH3 DOMAIN 1 | CH3 DOMAIN 2 | REFERENCES |
|---|---|---|---|---|
| Fc 1 | knobs-into-holes | T366Y | Y407T | Ridgway et al., 1996, |
| | (Y-T) | | | Protein Eng 9: 617-21 |
| Fc 2 | knobs-into-holes | S354C, T366W | Y349C, T366S, | Atwell et al., 1997, J |
| | (CW-CSAV) | | L368A, Y407V | Mol Biol. 270(1): 26-35; |
| | | | | Merchant et al., 1998, |
| | | | | Nat Biotechnol 16: |
| | | | | 677-681 |
| Fc 3 | HA-TF | S364H, F405A | Y349T, T394F | Moore et al., 2011, |
| | | | | MAbs 3(6): 546-57 |
| Fc 4 | ZW1 (VYAV-VLLW) | T350V, L351Y, | T350V, T366L, | Von Kreudenstein et |
| | | F405A, Y407V | K392L, T394W | al., 2013, MAbs 5: |
| | | | | 646-54 |
| Fc 5 | CH3 charge pairs | K392D, K409D | E356K, D399K | Gunasekaran et al., |
| | (DD-KK) | | | 2010, J Biol Chem |
| | | | | 285: 19637-46 |
| Fc 6 | IgG1 hingE, CH3 | IgG1: D221E, | IgG1: D221R, | Strop et al., 2012, J Mol |
| | charge pairs (EEE- | P228E, L368E | P228R, K409R | Biol 420: 204-19 |
| | RRR) | | | |
| Fc 7 | IgG2 hingE, CH3 | IgG2: C223E, | IgG2: C223R, | Strop et al., 2012, J Mol |
| | charge pairs (EEE- | P228E, L368E | E225R, P228R, | Biol 420: 204-19 |
| | RRRR) | | K409R | |
| Fc 8 | EW-RVT | K360E, K409W, | Q347R, D399V, | Choi et al., 2013, Mol |
| | | | F405T | Cancer Ther 12: 2748-59 |
| Fc 9 | EW-RVTS-S | K360E, K409W, | Q347R, D399V, | Choi et al., 2015, Mol |
| | | Y349C | F405T, S354C | Immunol 65: 377-83 |
| Fc 10 | Biclonic | 366K (+351K) | 351D or E or D at | Geuijen et al., 2014, |
| | | | 349, 368, 349, or | Journal of Clinical |
| | | | 349 + 355 | Oncology 32: suppl: 560 |
| Fc 11 | DuoBody (L-R) | F405L | K409R | Labrijn et al., 2013, |
| | | | | Proc Natl Acad Sci |
| | | | | USA 110: 5145-50 |
| Fc 12 | SEEDbody | IgG/A chimera | IgG/A chimera | Davis et al., 2010, |
| | | | | Protein Eng Des Sel |
| | | | | 23: 195-202 |
| Fc 13 | BEAT | residues from | residues from TCRβ | Moretti et al., 2013, |
| | | TCRα interface | interface | BMC Proceedings |
| | | | | 7(Suppl 6): O9 |
| Fc 14 | 7.8.60 (DMA-RRVV) | K360D, D399M, | E345R, Q347R, | Leaver-Fey et al., |
| | | Y407A | T366V, K409V | Structure 24: 641-51 |
| Fc 15 | 20.8.34 (SYMV- | Y349S, K370Y, | E356G, E357D, | Leaver-Fey et al., |
| | GDQA) | T366M, K409V | S364Q, Y407A | Structure 24: 641-51 |
| Fc 16 | Skew variant 12757 | None | none | FIG. 34 of US |
| | | | | 2016/0355600 |
| Fc 17 | Skew variant 12758 | L368D, K370S | S364K | FIG. 34 of US |
| | | | | 2016/0355600 |
| Fc 18 | Skew variant 12759 | L368D, K370S | S364K, E357L | FIG. 34 of US |
| | | | | 2016/0355600 |
| Fc 19 | Skew variant 12760 | L368D, K370S | S364K, E357Q | FIG. 34 of US |
| | | | | 2016/0355600 |
| Fc 20 | Skew variant 12761 | T411E, K360E, | D401K | FIG. 34 of US |
| | | Q362E | | 2016/0355600 |
| Fc 21 | Skew variant 12496 | L368E, K370S | S364K | FIG. 34 of US |
| | | | | 2016/0355600 |
| Fc 22 | Skew variant 12511 | K370S | S364K | FIG. 34 of US |
| | | | | 2016/0355600 |
| Fc 23 | Skew variant 12840 | L368E, K370S | S364K, E357Q | FIG. 34 of US |
| | | | | 2016/0355600 |
| Fc 24 | Skew variant 12841 | K370S | S364K, E357Q | FIG. 34 of US |
| | | | | 2016/0355600 |
| Fc 25 | Skew variant 12894 | L368E, K370S | S364K | FIG. 34 of US |
| | | | | 2016/0355600 |
| Fc 26 | Skew variant 12895 | K370S | S364K | FIG. 34 of US |
| | | | | 2016/0355600 |
| Fc 27 | Skew variant 12896 | L368E, K370S | S364K, E357Q | FIG. 34 of US |
| | | | | 2016/0355600 |
| Fc 28 | Skew variant 12901 | K370S | S364K, E357Q | FIG. 34 of US |
| | | | | 2016/0355600 |
| Fc 29 | pI_ISO(−) | I199T, N203D, | | FIG. 31 of US |
| | | K274Q, R355Q, | | 2016/0355600 |
| | | N384S, K392N, | | |
| | | V397M, Q419E, | | |
| | | DEL447 | | |
| Fc 30 | pI_(−)_Isosteric_A | N208D, Q295E, | | FIG. 31 of US |
| | | N384D, Q418E, | | 2016/0355600 |
| | | N421D | | |
| Fc 31 | pI_(−)_isosteric_B | N208D, Q295E, | | FIG. 31 of US |
| | | Q418E, N421D | | 2016/0355600 |
| Fc 32 | pI_ISO(+RR) | Q196K, I199T, | | FIG. 31 of US |
| | | P217R, P228R, | | 2016/0355600 |
| | | N276K | | |
| Fc 33 | pI_ISO(+) | Q196K, I199T, | | FIG. 31 of US |
| | | N276K | | 2016/0355600 |
| Fc 34 | pI_(+) isosteric_A | E269Q, E272Q, | | FIG. 31 of US |
| | | E283Q, E357Q, | | 2016/0355600 |
| Fc 35 | pI_(+)_isosteric_B | E269Q, E272Q, | | FIG. 31 of US |
| | | E283Q | | 2016/0355600 |
| Fc 36 | PI_(+) | E269Q, E272Q | | FIG. 31 of US |
| | isosteric_E269Q, | | | 2016/0355600 |
| | E272Q | | | |
| Fc 37 | pI_(+)_isosteric_E269Q, | E269Q, E283Q | | FIG. 31 of US |
| | E283Q | | | 2016/0355600 |
| Fc 38 | PI_(+) | E272Q, E283Q | | FIG. 31 of US |
| | isosteric_E2720, | | | 2016/0355600 |
| | E283Q | | | |
| Fc 39 | pI_(+)_isosteric_E269Q | E269Q | | FIG. 31 of US |
| | | | | 2016/0355600 |
| Fc 40 | Heterodimerization | F405A | T394F | FIG. 30A of US |
| | | | | 2016/0355600 |
| Fc 41 | Heterodimerization | S364D | Y349K | FIG. 30A of US |
| | | | | 2016/0355600 |
| Fc 42 | Heterodimerization | S364E | L368K | FIG. 30A of US |
| | | | | 2016/0355600 |
| Fc 43 | Heterodimerization | S364E | Y349K | FIG. 30A of US |
| | | | | 2016/0355600 |
| Fc 44 | Heterodimerization | S364F | K370G | FIG. 30A of US |
| | | | | 2016/0355600 |
| Fc 45 | Heterodimerization | S364H | Y349K | FIG. 30A of US |
| | | | | 2016/0355600 |
| Fc 46 | Heterodimerization | S364H | Y349T | FIG. 30A of US |
| | | | | 2016/0355600 |
| Fc 47 | Heterodimerization | S364Y | K370G | FIG. 30A of US |
| | | | | 2016/0355600 |
| Fc 48 | Heterodimerization | T411K | K370E | FIG. 30A of US |
| | | | | 2016/0355600 |
| Fc 49 | Heterodimerization | V397S, F405A | T394F | FIG. 30A of US |
| | | | | 2016/0355600 |
| Fc 50 | Heterodimerization | K370R, T411K | K370E, T411E | FIG. 30A of US |
| | | | | 2016/0355600 |
| Fc 51 | Heterodimerization | L351E, S364D | Y349K, L351K | FIG. 30A of US |
| | | | | 2016/0355600 |
| Fc 52 | Heterodimerization | L351E, S364E | Y349K, L351K | FIG. 30A of US |
| | | | | 2016/0355600 |
| Fc 53 | Heterodimerization | L351E, T366D | L351K, T366K | FIG. 30A of US |
| | | | | 2016/0355600 |
| Fc 54 | Heterodimerization | P395T, V397S, | T394F | FIG. 30A of US |
| | | F405A | | 2016/0355600 |
| Fc 55 | Heterodimerization | S364D, K370G | S364Y, K370R | FIG. 30A of US |
| | | | | 2016/0355600 |
| Fc 56 | Heterodimerization | S364D, T394F | Y349K, F405A | FIG. 30A of US |
| | | | | 2016/0355600 |
| Fc 57 | Heterodimerization | S364E, F405A | Y349K, T394F | FIG. 30A of US |
| | | | | 2016/0355600 |
| Fc 58 | Heterodimerization | S364E, F405S | Y349K, T394Y | FIG. 30A of US |
| | | | | 2016/0355600 |
| Fc 59 | Heterodimerization | S364E, T411E | Y349K, D401K | FIG. 30A of US |
| | | | | 2016/0355600 |
| Fc 60 | Heterodimerization | S364H, D401K | Y349T, T411E | FIG. 30A of US |
| | | | | 2016/0355600 |
| Fc 61 | Heterodimerization | S364H, F405A | Y349T, T394F | FIG. 30A of US |
| | | | | 2016/0355600 |
| Fc 62 | Heterodimerization | S364H, T394F | Y349T, F405A | FIG. 30A of US |
| | | | | 2016/0355600 |
| Fc 63 | Heterodimerization | Y349C, S364E | Y349K, S354C | FIG. 30A of US |
| | | | | 2016/0355600 |
| Fc 64 | Heterodimerization | L351E, S364D, | Y349K, L351K, | FIG. 30A of US |
| | | F405A | T394F | 2016/0355600 |
| Fc 65 | Heterodimerization | L351K, S364H, | Y349T, L351E, | FIG. 30A of US |
| | | D401K | T411E | 2016/0355600 |
| Fc 66 | Heterodimerization | S364E, T411E, | Y349K, T394F, | FIG. 30A of US |
| | | F405A | D401K | 2016/0355600 |
| Fc 67 | Heterodimerization | S364H, D401K, | Y349T, T394F, | FIG. 30A of US |
| | | F405A | T411E | 2016/0355600 |
| Fc 68 | Heterodimerization | S364H, F405A, | Y349T, T394F, | FIG. 30A of US |
| | | T411E | D401K | 2016/0355600 |
| Fc 69 | Heterodimerization | T411E, K360E, | D401K | FIG. 30C of US |
| | | N390D | | 2016/0355600 |
| Fc 70 | Heterodimerization | T411E, Q362E, | D401K | FIG. 30C of US |
| | | N390D | | 2016/0355600 |
| Fc 71 | Heterodimerization | T411E, Q347R | D401K, K360D | FIG. 30C of US |
| | | | | 2016/0355600 |
| Fc 72 | Heterodimerization | T411E, Q347R | D401K, K360E | FIG. 30C of US |
| | | | | 2016/0355600 |
| Fc 73 | Heterodimerization | T411E, K360 | D401K, Q347K | FIG. 30C of US |
| | | | | 2016/0355600 |
| Fc 74 | Heterodimerization | T411E, K360D | D401K, Q347R | FIG. 30C of US |
| | | | | 2016/0355600 |
| Fc 75 | Heterodimerization | T411E, K360E | D401K, Q347K | FIG. 30C of US |
| | | | | 2016/0355600 |
| Fc 76 | Heterodimerization | T411E, K360E | D401K, Q347R | FIG. 30C of US |
| | | | | 2016/0355600 |
| Fc 77 | Heterodimerization | T411E, S364K | D401K, K370S | FIG. 30C of US |
| | | | | 2016/0355600 |
| Fc 78 | Heterodimerization | T411E, K370S | D401K, S364K | FIG. 30C of US |
| | | | | 2016/0355600 |
| Fc 79 | Heterodimerization | Q347E | E357Q | FIG. 30C of US |
| | | | | 2016/0355600 |
| Fc 80 | Heterodimerization | Q347E | E357Q, Q362K | FIG. 30C of US |
| | | | | 2016/0355600 |
| Fc 81 | Heterodimerization | K360D, Q362E | Q347R | FIG. 30C of US |
| | | | | 2016/0355600 |
| Fc 82 | Heterodimerization | K360D, Q362E | D401K | FIG. 30C of US |
| | | | | 2016/0355600 |
| Fc 83 | Heterodimerization | K360D, Q362E | Q347R, D401K | FIG. 30C of US |
| | | | | 2016/0355600 |
| Fc 84 | Heterodimerization | K360E, Q362E | Q347R | FIG. 30C of US |
| | | | | 2016/0355600 |
| Fc 85 | Heterodimerization | K360E, Q362E | D401K | FIG. 30C of US |
| | | | | 2016/0355600 |
| Fc 86 | Heterodimerization | K360E, Q362E | Q347R, D401K | FIG. 30C of US |
| | | | | 2016/0355600 |
| Fc 87 | Heterodimerization | Q362E, N390D | D401K | FIG. 30C of US |
| | | | | 2016/0355600 |
| Fc 88 | Heterodimerization | Q347E, K360D | D401N | FIG. 30C of US |
| | | | | 2016/0355600 |
| Fc 89 | Heterodimerization | K360D | Q347R, N390K | FIG. 30C of US |
| | | | | 2016/0355600 |
| Fc 90 | Heterodimerization | K360D | N390K, D401N | FIG. 30C of US |
| | | | | 2016/0355600 |
| Fc 91 | Heterodimerization | K360E | Y349H | FIG. 30C of US |
| | | | | 2016/0355600 |
| Fc 92 | Heterodimerization | K370S, Q347E | S364K | FIG. 30C of US |
| | | | | 2016/0355600 |
| Fc 93 | Heterodimerization | K370S, E357L | S364K | FIG. 30C of US |
| | | | | 2016/0355600 |
| Fc 94 | Heterodimerization | K370S, E357Q | S364K | FIG. 30C of US |
| | | | | 2016/0355600 |
| Fc 95 | Heterodimerization | K370S, Q347E, | S364K | FIG. 30C of US |
| | | E357L | | 2016/0355600 |
| Fc 96 | Heterodimerization | K370S, Q347E, | S364K | FIG. 30C of US |
| | | E357Q | | 2016/0355600 |
| Fc 97 | Heterodimerization | L368D, K370S, | S364K | FIG. 30D of US |
| | | Q347E | | 2016/0355600 |
| Fc 98 | Heterodimerization | L368D, K370S, | S364K | FIG. 30D of US |
| | | E357L | | 2016/0355600 |
| Fc 99 | Heterodimerization | L368D, K370S, | S364K | FIG. 30D of US |
| | | E357Q | | 2016/0355600 |
| Fc 100 | Heterodimerization | L368D, K370S, | S364K | FIG. 30D of US |
| | | Q347E, E357L | | 2016/0355600 |
| Fc 101 | Heterodimerization | L368D, K370S, | S364K | FIG. 30D of US |
| | | Q347E, E357Q | | 2016/0355600 |
| Fc 102 | Heterodimerization | L368E, K370S, | S364K | FIG. 30D of US |
| | | Q347E | | 2016/0355600 |
| Fc 103 | Heterodimerization | L368E, K370S, | S364K | FIG. 30D of US |
| | | E357L | | 2016/0355600 |
| Fc 104 | Heterodimerization | L368E, K370S, | S364K | FIG. 30D of US |
| | | E357Q | | 2016/0355600 |
| Fc 105 | Heterodimerization | L368E, K370S, | S364K | FIG. 30D of US |
| | | Q347E, E357L | | 2016/0355600 |
| Fc 106 | Heterodimerization | L368E, K370S, | S364K | FIG. 30D of US |
| | | Q347E, E357Q | | 2016/0355600 |
| Fc 107 | Heterodimerization | L368D, K370T, | S364K | FIG. 30D of US |
| | | Q347E | | 2016/0355600 |
| Fc 108 | Heterodimerization | L368D, K370T, | S364K | FIG. 30D of US |
| | | E357L | | 2016/0355600 |
| Fc 109 | Heterodimerization | L368D, K370T, | S364K | FIG. 30D of US |
| | | E357Q | | 2016/0355600 |
| Fc 110 | Heterodimerization | L368D, K370T, | S364K | FIG. 30D of US |
| | | Q347E, E357L | | 2016/0355600 |
| Fc 111 | Heterodimerization | L368D, K370T, | S364K | FIG. 30D of US |
| | | Q347E, E357Q | | 2016/0355600 |
| Fc 112 | Heterodimerization | L368E, K370T, | S364K | FIG. 30D of US |
| | | Q347E | | 2016/0355600 |
| Fc 113 | Heterodimerization | L368E, K370T, | S364K | FIG. 30D of US |
| | | E357L | | 2016/0355600 |
| Fc 114 | Heterodimerization | L368E, K370T, | S364K | FIG. 30D of US |
| | | E357Q | | 2016/0355600 |
| Fc 115 | Heterodimerization | L368E, K370T, | S364K | FIG. 30D of US |
| | | Q347E, E357L | | 2016/0355600 |
| Fc 116 | Heterodimerization | L368E, K370T, | S364K | FIG. 30D of US |
| | | Q347E, E357Q | | 2016/0355600 |
| Fc 117 | Heterodimerization | T411E, Q362E | D401K, T411K | FIG. 30D of US |
| | | | | 2016/0355600 |
| Fc 118 | Heterodimerization | T411E, N390D | D401K, T411K | FIG. 30D of US |
| | | | | 2016/0355600 |
| Fc 119 | Heterodimerization | T411E, Q362E | D401R, T411R | FIG. 30D of US |
| | | | | 2016/0355600 |
| Fc 120 | Heterodimerization | T411E, N390D | D401R, T411R | FIG. 30D of US |
| | | | | 2016/0355600 |
| Fc 121 | Heterodimerization | Y407T | T366Y | FIG. 30D of US |
| | | | | 2016/0355600 |
| Fc 122 | Heterodimerization | F405A | T394W | FIG. 30D of US |
| | | | | 2016/0355600 |
| Fc 123 | Heterodimerization | T366Y, F405A | T394W, Y407T | FIG. 30D of US |
| | | | | 2016/0355600 |
| Fc 124 | Heterodimerization | T3665, L368A, | T366W | FIG. 30D of US |
| | | Y407V | | 2016/0355600 |
| Fc 125 | Heterodimerization | T366S, L368A, | T366W, S354C | FIG. 30D of US |
| | | Y407V, Y349C | | 2016/0355600 |
| Fc 126 | Heterodimerization | K392D, K409D | E356K.D399K | FIG. 30E of US |
| | | | | 2016/0355600 |
| Fc 127 | Heterodimerization | K370D, K392D, | E356K, E357K, | FIG. 30E of US |
| | | K409D | D399K | 2016/0355600 |
| Fc 128 | Heterodimerization | I199T, N203D, | Q196K, L99T, | FIG. 30E of US |
| | | K247Q, R355Q, | P217R, P228R, | 2016/0355600 |
| | | N384S, K392N, | N276K | |
| | | V397M, Q419E, | | |
| | | K447 | | |
| Fc 129 | Heterodimerization | I199T, N203D, | Q196K, L99T, | FIG. 30E of US |
| | | K247Q, R355Q, | N276K | 2016/0355600 |
| | | N384S, K392N, | | |
| | | V397M, Q419E, | | |
| | | K447 | | |
| Fc 130 | Heterodimerization | N384S, K392N, | N276K | FIG. 30E of US |
| | | V397M, Q419E | | 2016/0355600 |
| Fc 131 | Heterodimerization | D221E, P228E, | D221R, P228R, | FIG. 30E of US |
| | | L368E | K409R | 2016/0355600 |
| Fc 132 | Heterodimerization | C220E, P228E, | C220R, E224R, | FIG. 30E of US |
| | | L368E | P228R, K409R | 2016/0355600 |
| Fc 133 | Heterodimerization | F405L | K409R | FIG. 30E of US |
| | | | | 2016/0355600 |
| Fc 134 | Heterodimerization | T366I, K392M, | F405A, Y407V | FIG. 30E of US |
| | | T394W | | 2016/0355600 |
| Fc 135 | Heterodimerization | T366V, K409F | L351Y, Y407A | FIG. 30E of US |
| | | | | 2016/0355600 |
| Fc 136 | Heterodimerization | T366A, K392E, | D399R, S400R, | FIG. 30E of US |
| | | K409F, T411E | Y407A | 2016/0355600 |
| Fc 137 | Heterodimerization | L351K | L351E | FIG. 30E of US |
| | | | | 2016/0355600 |
| Fc 138 | Heterodimerization | I199T, N203D, | Q196K, L199T, | FIG. 30E of US |
| | | K247Q, R355Q, | P217R, P228R, | 2016/0355600 |
| | | Q419E, K447 | N276K | |
| Fc 139 | Heterodimerization | I199T, N203D, | Q196K, I199T, | FIG. 30E of US |
| | | K247Q, R355Q, | N276K | 2016/0355600 |
| | | Q419E, K447 | | |
| Fc 140 | Heterodimerization | I199T, N203D, | | FIG. 30E of US |
| | | K274Q, R355Q, | | 2016/0355600 |
| | | N384S, K392N, | | |
| | | V397M, Q419E | | |
| | | DEL447 | | |
| Fc 141 | Heterodimerization | N208D, Q295E | | FIG. 30E of US |
| | | N384D, Q418E | | 2016/0355600 |
| | | N421D | | |
| Fc 142 | Heterodimerization | N208D, Q295E | | FIG. 30E of US |
| | | Q418E, N421D | | 2016/0355600 |
| Fc 143 | Heterodimerization | Q196K, I199T | | FIG. 30E of US |
| | | P217R, P228R | | 2016/0355600 |
| | | N276K | | |
| Fc 144 | Heterodimerization | Q196K, I199T | | FIG. 30E of US |
| | | N276K | | 2016/0355600 |
| Fc 145 | Heterodimerization | E269Q, E272Q | | FIG. 30E of US |
| | | E283Q, E357Q | | 2016/0355600 |
| Fc 146 | Heterodimerization | E269Q, E272Q | | FIG. 30E of US |
| | | E283Q, | | 2016/0355600 |
| Fc 147 | Heterodimerization | E269Q, E272Q | | FIG. 30E of US |
| | | | | 2016/0355600 |
| Fc 148 | Heterodimerization | E269Q, E283Q | | FIG. 30E of US |
| | | | | 2016/0355600 |
| Fc 149 | Heterodimerization | E272Q, E283Q | | FIG. 30E of US |
| | | | | 2016/0355600 |
| Fc 150 | Heterodimerization | E269Q | | FIG. 30E of US |
| | | | | 2016/0355600 |

### 7.3.1.5.1. Steric Variants

MBMs (e.g., BBMs) can comprise one or more, e.g., a plurality, of modifications to one or more of the constant domains of an Fc domain, e.g., to the CH3 domains. In one example, a MBM (e.g., a BBM) comprises two polypeptides that each comprise a heavy chain constant domain of an antibody, e.g., a CH2 or CH3 domain. In an example, the two heavy chain constant domains, e.g., the CH2 or CH3 domains of the MBM (e.g., BBM) comprise one or more modifications that allow for a heterodimeric association between the two chains. In one aspect, the one or more modifications are disposed on CH2 domains of the two heavy chains. In one aspect, the one or more modifications are disposed on CH3 domains of at least two polypeptides of the MBM.

One mechanism for Fc heterodimerization is generally referred to in the art as “knobs and holes”, or “knob-in-holes”, or “knobs-into-holes”. These terms refer to amino acid mutations that create steric influences to favor formation of Fc heterodimers over Fc homodimers, as described in, e.g., Ridgway et al., 1996, Protein Engineering 9(7):617; Atwell et al., 1997, J. Mol. Biol. 270:26; U.S. Pat. No. 8,216,805. Knob-in-hole mutations can be combined with other strategies to improve heterodimerization.

In one aspect, the one or more modifications to a first polypeptide of the MBM comprising a heavy chain constant domain can create a “knob” and the one or more modifications to a second polypeptide of the MBM creates a “hole,” such that heterodimerization of the polypeptide of the MBM comprising a heavy chain constant domain causes the “knob” to interface (e.g., interact, e.g., a CH2 domain of a first polypeptide interacting with a CH2 domain of a second polypeptide, or a CH3 domain of a first polypeptide interacting with a CH3 domain of a second polypeptide) with the “hole.” The “knob” projects from the interface of a first polypeptide of the MBM comprising a heavy chain constant domain and is therefore positionable in a compensatory “hole” in the interface with a second polypeptide of the MBM comprising a heavy chain constant domain so as to stabilize the heteromultimer, and thereby favor heteromultimer formation over homomultimer formation, for example. The knob can exist in the original interface or can be introduced synthetically (e.g. by altering nucleic acid encoding the interface). The import residues for the formation of a knob are generally naturally occurring amino acid residues and can be selected from arginine (R), phenylalanine (F), tyrosine (Y) and tryptophan (W). In some cases, tryptophan and tyrosine are selected. In an embodiment, the original residue for the formation of the protuberance has a small side chain volume, such as alanine, asparagine, aspartic acid, glycine, serine, threonine or valine.

A “hole” comprises at least one amino acid side chain which is recessed from the interface of a second polypeptide of the MBM comprising a heavy chain constant domain and therefore accommodates a corresponding knob on the adjacent interfacing surface of a first polypeptide of the MBM comprising a heavy chain constant domain. The hole can exist in the original interface or can be introduced synthetically (e.g. by altering nucleic acid encoding the interface). The import residues for the formation of a hole are usually naturally occurring amino acid residues and are in some embodiments selected from alanine (A), serine (S), threonine (T) and valine (V). In one embodiment, the amino acid residue is serine, alanine or threonine. In another embodiment, the original residue for the formation of the hole has a large side chain volume, such as tyrosine, arginine, phenylalanine or tryptophan.

In an embodiment, a first CH3 domain is modified at residue 366, 405 or 407 to create either a “knob” or a hole” (as described above), and the second CH3 domain that heterodimerizes with the first CH3 domain is modified at: residue 407 if residue 366 is modified in the first CH3 domain, residue 394 if residue 405 is modified in the first CH3 domain, or residue 366 if residue 407 is modified in the first CH3 domain to create a “hole” or “knob” complementary to the “knob” or “hole” of the first CH3 domain.

In another embodiment, a first CH3 domain is modified at residue 366, and the second CH3 domain that heterodimerizes with the first CH3 domain is modified at residues 366, 368 and/or 407, to create a “hole” or “knob” complementary to the “knob” or “hole” of the first CH3 domain. In one embodiment, the modification to the first CH3 domain introduces a tyrosine (Y) residue at position 366. In an embodiment, the modification to the first CH3 is T366Y. In one embodiment, the modification to the first CH3 domain introduces a tryptophan (W) residue at position 366. In an embodiment, the modification to the first CH3 is T366W. In some embodiments, the modification to the second CH3 domain that heterodimerizes with the first CH3 domain modified at position 366 (e.g., has a tyrosine (Y) or tryptophan (W) introduced at position 366, e.g., comprises the modification T366Y or T366W), comprises a modification at position 366, a modification at position 368 and a modification at position 407. In some embodiments, the modification at position 366 introduces a serine (S) residue, the modification at position 368 introduces an alanine (A), and the modification at position 407 introduces a valine (V). In some embodiments, the modifications comprise T366S, L368A and Y407V. In one embodiment, the first CH3 domain of the multispecific molecule comprises the modification T366Y, and the second CH3 domain that heterodimerizes with the first CH3 domain comprises the modifications T366S, L368A and Y407V, or vice versa. In one embodiment, the first CH3 domain of the multispecific molecule comprises the modification T366W, and the second CH3 domain that heterodimerizes with the first CH3 domain comprises the modifications T366S, L368A and Y407V, or vice versa.

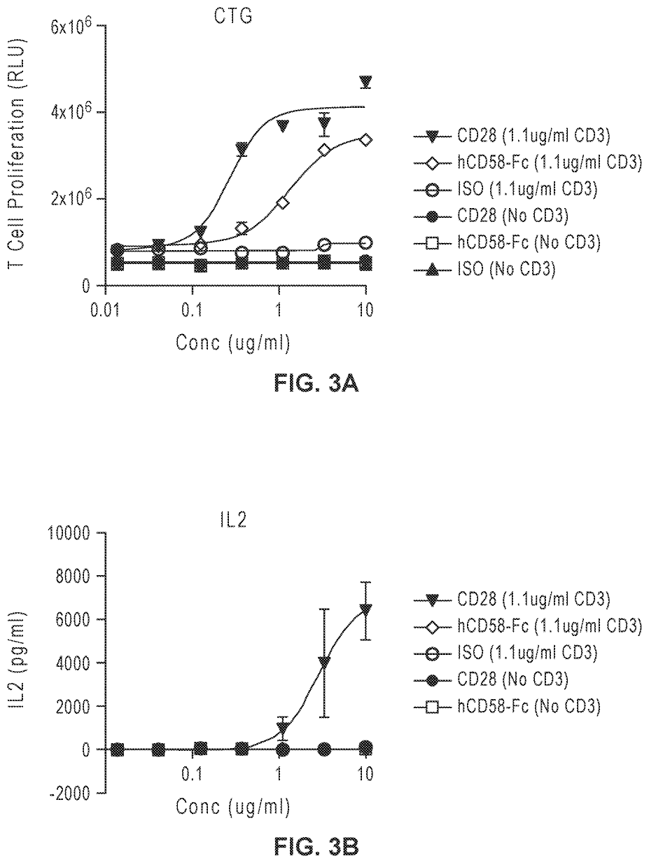

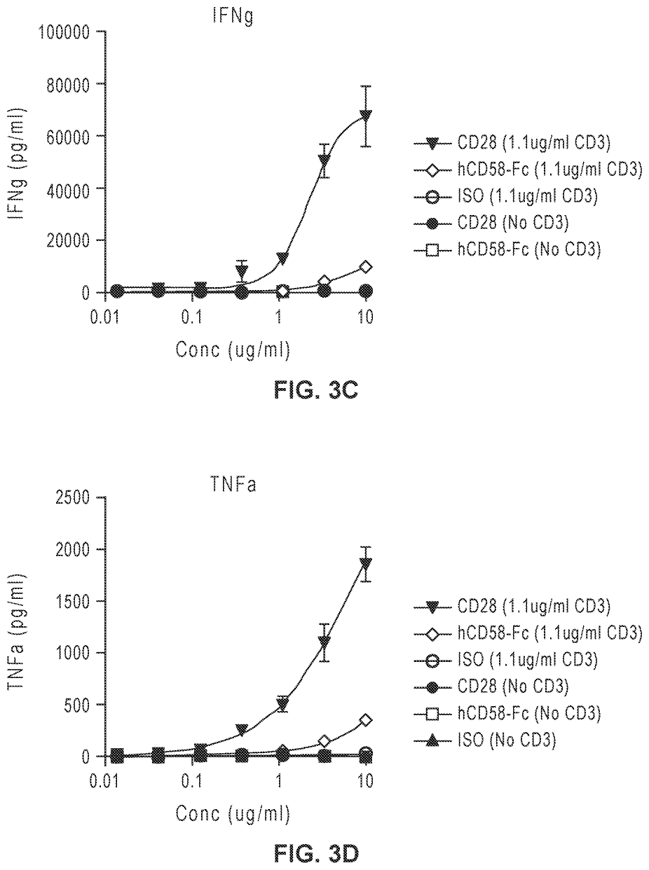

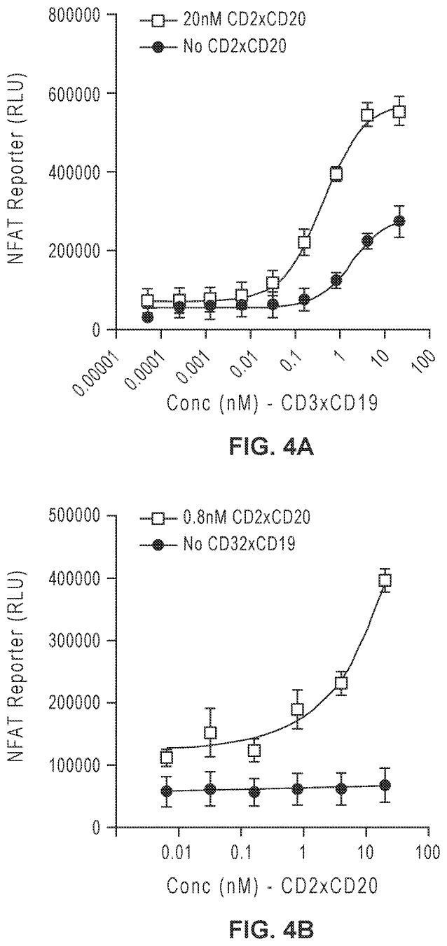

Additional steric or “skew” (e.g., knob-in-hole) modifications are described in PCT publication no. WO2014/145806 (for example, FIG. 3, FIG. 4 and FIG. 12 of WO2014/145806), PCT publication no. WO2014/110601, and PCT publication no. WO 2016/086186, WO 2016/086189, WO 2016/086196 and WO 2016/182751. An example of a KIH variant comprises a first constant chain comprising a L368D and a K370S modification, paired with a second constant chain comprising a S364K and E357Q modification.

Additional knob-in-hole modification pairs suitable for use in any of the MBMs of the present disclosure are further described in, for example, WO1996/027011, and Merchant et al., 1998, Nat. Biotechnol., 16:677-681.

In further embodiments, the CH3 domains can be additionally modified to introduce a pair of cysteine residues. Without being bound by theory, it is believed that the introduction of a pair of cysteine residues capable of forming a disulfide bond provide stability to heterodimerized MBMs (e.g., BBMs) comprising paired CH3 domains. In some embodiments, the first CH3 domain comprises a cysteine at position 354, and the second CH3 domain that heterodimerizes with the first CH3 domain comprises a cysteine at position 349. In some embodiments, the first CH3 domain comprises a cysteine at position 354 (e.g., comprises the modification S354C) and a tyrosine (Y) at position 366 (e.g., comprises the modification T366Y), and the second CH3 domain that heterodimerizes with the first CH3 domain comprises a cysteine at position 349 (e.g., comprises the modification Y349C), a serine at position 366 (e.g., comprises the modification T366S), an alanine at position 368 (e.g., comprises the modification L368A), and a valine at position 407 (e.g., comprises the modification Y407V). In some embodiments, the first CH3 domain comprises a cysteine at position 354 (e.g., comprises the modification S354C) and a tryptophan (W) at position 366 (e.g., comprises the modification T366W), and the second CH3 domain that heterodimerizes with the first CH3 domain comprises a cysteine at position 349 (e.g., comprises the modification Y349C), a serine at position 366 (e.g., comprises the modification T366S), an alanine at position 368 (e.g., comprises the modification L368A), and a valine at position 407 (e.g., comprises the modification Y407V).

An additional mechanism that finds use in the generation of heterodimers is sometimes referred to as “electrostatic steering” as described in Gunasekaran et al., 2010, J. Biol. Chem. 285(25):19637. This is sometimes referred to herein as “charge pairs”. In this embodiment, electrostatics are used to skew the formation towards heterodimerization. As a skilled artisan will appreciate, these can also have an effect on pI, and thus on purification, and thus could in some cases also be considered pI variants. However, as these were generated to force heterodimerization and were not used as purification tools, they are classified as “steric variants”. These include, but are not limited to, D221E/P228E/L368E paired with D221R/P228R/K409R and C220E/P228E/368E paired with C220R/E224R/P228R/K409R.

Additional variants that can be combined with other variants, optionally and independently in any amount, such as pI variants outlined herein or other steric variants that are shown in FIG. 37 of US 2012/0149876.

In some embodiments, the steric variants outlined herein can be optionally and independently incorporated with any pI variant (or other variants such as Fc variants, FcRn variants) into one or both Fc regions, and can be independently and optionally included or excluded from the MBMs of the disclosure.

A list of suitable skew variants is found in Table 4 showing some pairs of particular utility in many embodiments. Of particular use in many embodiments are the pairs of sets including, but not limited to, S364K/E357Q:L368D/K370S; L368D/K370S:S364K; L368E/K370S:S364K; T411T/E360E/Q362E:D401K; L368D/K370S:S364K/E357L; and K370S:S364K/E357Q. In terms of nomenclature, the pair “S364K/E357Q:L368D/K370S” means that one of the Fc regions has the double variant set S364K/E357Q and the other has the double variant set L368D/K370S.

TABLE 4
Exemplary skew variants

| Fc region 1 | Fc region 2 |
|---|---|
| F405A | T394F |
| S364D | Y349K |
| S364E | L368K |
| S364E | Y349K |
| S364F | K370G |
| S364H | Y349K |
| S364H | Y349T |
| S364Y | K370G |
| T411K | K370E |
| V397S/F405A | T394F |
| K370R/T411K | K370E/T411E |
| L351E/S364D | Y349K/L351K |
| L351E/S364E | Y349K/L351K |
| L351E/T366D | L351K/T366K |
| P395T/V397S/F405A | T394F |
| S364D/K370G | S364Y/K370R |
| S364D/T394F | Y349K/F405A |
| S364E/F405A | Y349K/T394F |
| S364E/F405S | Y349K/T394Y |
| S364E/T411E | Y349K/D401K |
| S364H/D401K | Y349T/T411E |
| S364H/F405A | Y349T/T394F |
| S364H/T394F | Y349T/F405A |
| Y349C/S364E | Y349K/S354C |
| L351E/S364D/F405A | Y349K/L351K/T394F |
| L351K/S364H/D401K | Y349T/L351E/T411E |
| S364E/T411E/F405A | Y349K/T394F/D401K |
| S364H/D401K/F405A | Y349T/T394F/T411E |
| S364H/F405A/T411E | Y349T/T394F/D401K |
| K370E/T411D | T411K |
| L368E/K409E | L368K |
| Y349T/T394F/S354C | S364H/F405A/Y349C |
| T411E | D401K |
| T411E | D401R/T411R |
| Q347E/K360E | Q347R |
| L368E | S364K |
| L368E/K370S | S364K |
| L368E/K370T | S364K |
| L368E/D401R | S364K |
| L368E/D401N | S364K |
| L368E | E357S/S364K |
| L368E | S364K/K409E |
| L368E | S364K/K409V |
| L368D | S364K |
| L368D/K370S | S364K |
| L368D/K370S | S364K/E357L |
| L368D/K370S | S364K/E357Q |
| T411E/K360E/Q362E | D401K |
| K370S | S364K |
| L368E/K370S | S364K/E357Q |
| K370S | S364K/E357Q |
| T411E/K360D | D401K |
| T411E/K360E | D401K |
| T411E/Q362E | D401K |
| T411E/N390D | D401K |
| T411E | D401K/Q347K |
| T411E | D401K/Q347R |
| T411E/K360D/Q362E | D401K |
| K392D/K409D | E356K/D399K |
| K370D/K392D/K409D | E356K/E357K/D399K |
| I199T/N203D/K247Q/R355Q/N384S/ | Q196K/I199T/P217R/P228R/N276K |
| K392N/V397M/Q419E/K447_{—} | |
| I199T/N203D/K247Q/R355Q/N384S/ | Q196K/I199T/N276K |
| K392N/V397M/Q419E/K447_{—} | |
| N384S/K392N/V397M/Q419E | N276K |
| D221E/P228E/L368E | D221R/P228R/K409R |
| C220E/P228E/L368E | C220R/E224R/P228R/ |
| | K409R |
| F405L | K409R |
| T366I/K392M/T394W | F405A/Y407V |
| T366V/K409F | L351Y/Y407A |
| T366A/K392E/K409F/T411E | D399R/S400R/Y407A |
| L351K | L351E |
| I199T/N203D/K247Q/R355Q/Q419E/K447_{—} | Q196K/I199T/P217R/P228R/N276K |
| I199T/N203D/K247Q/R355Q/Q419E/K447_{—} | Q196K/I199T/N276K |
| I199T N203D K274Q R355Q N384S | |
| K392N V397M Q419E DEL447 | |
| N208D Q295E N384D Q418E N421D | |
| N208D Q295E Q418E N421D | |
| Q196K I199T P217R P228R N276K | |
| Q196K I199T N276K | |
| E269Q E272Q E283Q E357Q | |
| E269Q E272Q E283Q | |
| E269Q E272Q | |
| E269Q E283Q | |
| E272Q E283Q | |
| E269Q | |
| T411E/K360E/N390D | D401K |
| T411E/Q362E/N390D | D401K |
| T411E/Q347R | D401K/K360D |
| T411E/Q347R | D401K/K360E |
| T411E/K360 | D401K/Q347K |
| T411E/K360D | D401K/Q347R |
| T411E/K360E | D401K/Q347K |
| T411E/K360E | D401K/Q347R |
| T411E/S364K | D401K/K370S |
| T411E/K370S | D401K/S364K |
| Q347E | E357Q |
| Q347E | E357Q/Q362K |
| K360D/Q362E | Q347R |
| K360D/Q362E | D401K |
| K360D/Q362E | Q347R/D401K |
| K360E/Q362E | Q347R |
| K360E/Q362E | D401K |
| K360E/Q362E | Q347R/D401K |
| Q362E/N390D | D401K |
| Q347E/K360D | D401N |
| K360D | Q347R/N390K |
| K360D | N390K/D401N |
| K360E | Y349H |
| K370S/Q347E | S364K |
| K370S/E357L | S364K |
| K370S/E357Q | S364K |
| K370S/Q347E/E357L | S364K |
| K370S/Q347E/E357Q | S364K |
| L368D/K370S/Q347E | S364K |
| L368D/K370S/E357L | S364K |
| L368D/K370S/E357Q | S364K |
| L368D/K370S/Q347E/E357L | S364K |
| L368D/K370S/Q347E/E357Q | S364K |
| L368E/K370S/Q347E | S364K |
| L368E/K370S/E357L | S364K |
| L368E/K370S/E357Q | S364K |
| L368E/K370S/Q347E/E357L | S364K |
| L368E/K370S/Q347E/E357Q | S364K |
| L368D/K370T/Q347E | S364K |
| L368D/K370T/E357L | S364K |
| L368D/K370T/E357Q | S364K |
| L368D/K370T/Q347E/E357L | S364K |
| L368D/K370T/Q347E/E357Q | S364K |
| L368E/K370T/Q347E | S364K |
| L368E/K370T/E357L | S364K |
| L368E/K370T/E357Q | S364K |
| L368E/K370T/Q347E/E357L | S364K |
| L368E/K370T/Q347E/E357Q | S364K |
| T411E/Q362E | D401K/T411K |
| T411E/N390D | D401K/T411K |
| T411E/Q362E | D401R/T411R |
| T411E/N390D | D401R/T411R |
| Y407T | T366Y |
| F405A | T394W |
| T366Y/F405A | T394W/Y407T |
| Y407A | T366W |
| T366S/L368A/Y407V | T366W |
| T366S/L368A/Y407V/Y349C | T366W/S354C |
| K392D/K409D | E356K/D399K |
| K370D/K392D/K409D | E356K/E357K/D399K |
| I199T/N203D/K247Q/R355Q/N384S/ | Q196K/I199T/P217R/P228R/N276K |
| K392N/V397M/Q419E/K447_{—} | |
| I199T/N203D/K247Q/R355Q/N384S/ | Q196K/I199T/N276K |
| K392N/V397M/Q419E/K447_{—} | |
| N384S/K392N/V397M/Q419E | N276K |
| D221E/P228E/L368E | D221R/P228R/K409R |
| C220E/P228E/L368E | C220R/E224R/P228R/ |
| | K409R |
| F405L | K409R |
| T366I/K392M/T394W | F405A/Y407V |
| T366V/K409F | L351Y/Y407A |
| T366A/K392E/K409F/T411E | D399R/S400R/Y407A |
| L351K | L351E |
| I199T/N203D/K247Q/R355Q/Q419E/K447_{—} | Q196K/I199T/P217R/P228R/N276K |
| I199T/N203D/K247Q/R355Q/Q419E/K447_{—} | Q196K/I199T/N276K |
| I199T N203D K274Q R355Q N384S | |
| K392N V397M Q419E DEL447 | |
| N208D Q295E N384D Q418E N421D | |
| Q295E N384D Q418E N421D | |
| N208D Q295E Q418E N421D | |
| Q295E Q418E N421D | |
| Q196K I199T P217R P228R N276K | |
| Q196K I199T N276K | |
| E269Q E272Q E283Q E357Q | |
| E269Q E272Q E283Q | |
| E269Q E272Q | |
| E269Q E283Q | |
| E272Q E283Q | |
| E269Q | |

In some embodiments, a MBM comprises a first Fc region and a second Fc region. In some embodiments, the first Fc region comprises the following mutations: L368D and K370S, and the second Fc region comprises the following mutations: S364K and E357Q. In some embodiments, the first Fc region comprises the following mutations: S364K and E357Q, and the second Fc region comprises the following mutations: L368D and K370S.

### 7.3.1.5.2. Alternative Knob and Hole: IgG Heterodimerization

Heterodimerization of polypeptide chains of a MBM (e.g., a BBM) comprising paired CH3 domains can be increased by introducing one or more modifications in a CH3 domain which is derived from the IgG1 antibody class. In an embodiment, the modifications comprise a K409R modification to one CH3 domain paired with F405L modification in the second CH3 domain. Additional modifications can also, or alternatively, be at positions 366, 368, 370, 399, 405, 407, and 409. In some cases, heterodimerization of polypeptides comprising such modifications is achieved under reducing conditions, e.g., 10-100 mM 2-MEA (e.g., 25, 50, or 100 mM 2-MEA) for 1-10, e.g., 1.5-5, e.g., 5, hours at 25-37 C, e.g., 25 C or 37 C.

The amino acid replacements described herein can be introduced into the CH3 domains using techniques which are well known (see, e.g., McPherson, ed., 1991, Directed Mutagenesis: a Practical Approach; Adelman et al., 1983, DNA, 2:183).

The IgG heterodimerization strategy is further described in, for example, WO2008/119353, WO2011/131746, and WO2013/060867.

In any of the embodiments described in this Section, the CH3 domains can be additionally modified to introduce a pair of cysteine residues as described in Section 7.3.1.3.

### 7.3.1.5.3. pI (Isoelectric Point) Variants

In general, as will be appreciated by a skilled artisan, there are two general categories of pI variants: those that increase the pI of the protein (basic changes) and those that decrease the pI of the protein (acidic changes). As described herein, all combinations of these variants can be done: one Fc region can be wild type, or a variant that does not display a significantly different pI from wild-type, and the other can be either more basic or more acidic. Alternatively, each Fc region is changed, one to more basic and one to more acidic.

Exemplary combinations of pI variants are shown in Table 5. As outlined herein and shown in Table 5, these changes are shown relative to IgG1, but all isotypes can be altered this way, as well as isotype hybrids. In the case where the heavy chain constant domain is from IgG2-4, R133E and R133Q can also be used.

TABLE 5
Exemplary pl Variant Combinations

| Variant constant region | Substitutions |
|---|---|
| pl_ISO(−) | I199T N203D K274Q R355Q N384S |
| | K392N V397M Q419E DEL447 |
| pl_(−)_isosteric_A | N208D Q295E N384D Q418E N421D |
| pl_(−)_isosteric A-Fc | Q295E N384D Q418E N421D |
| only | |
| pl_(−)_isosteric_B | N208D Q295E Q418E N421D |
| pl_(−)_isosteric_B-Fc | Q295E Q418E N421D |
| only | |
| pl_ISO(+RR) | Q196K I199T P217R P228R N276K |
| pl_ISO(+) | Q196K I199T N276K |
| pl_(+)_isosteric_A | E269Q E272Q E283Q E357Q |
| pl_(+)_isosteric_B | E269Q E272Q E283Q |
| pl_(+)_isosteric_E269Q/ | E269Q E272Q |
| E272Q | |
| pl_(+)_isosteric_E269Q/ | E269Q E283Q |
| E283Q | |
| pl_(+)_isosteric_E272Q/ | E272Q E283Q |
| E283Q | |
| pl_(+)_isosteric_E269Q | E269Q |

In one embodiment, a combination of pI variants has one Fc region (the negative Fab side) comprising 208D/295E/384D/418E/421D variants (N208D/Q295E/N384D/Q418E/N421D when relative to human IgG1) and a second Fc region (the positive scFv side) comprising a positively charged scFv linker, e.g., L36 (described in Section 7.3.3). However, as will be appreciated by a skilled artisan, the first Fc region includes a CH1 domain, including position 208. Accordingly, in constructs that do not include a CH1 domain (for example for MBMs that do not utilize a CH1 domain as one of the domains, for example in a format depicted in FIG. 1K), a negative pI variant Fc set can include 295E/384D/418E/421D variants (Q295E/N384D/Q418E/N421D when relative to human IgG1).

In some embodiments, a first Fc region has a set of substitutions from Table 5 and a second Fc region is connected to a charged linker (e.g., selected from those described in Section 7.3.3).

In some embodiments, a MBM comprises a first Fc region and a second Fc region. In some embodiments, the first Fc region comprises the following mutations: N208D, Q295E, N384D, Q418E, and N421D. In some embodiments, the second Fc region comprises the following mutations: N208D, Q295E, N384D, Q418E, and N421D.

### 7.3.1.5.4. Isotopic Variants

In addition, many embodiments of the disclosure rely on the “importation” of pI amino acids at particular positions from one IgG isotype into another, thus reducing or eliminating the possibility of unwanted immunogenicity being introduced into the variants. A number of these are shown in FIG. 21 of US Publ. 2014/0370013. That is, IgG1 is a common isotype for therapeutic antibodies for a variety of reasons, including high effector function. However, the heavy constant region of IgG1 has a higher pI than that of IgG2 (8.10 versus 7.31). By introducing IgG2 residues at particular positions into the IgG1 backbone, the pI of the resulting Fc region is lowered (or increased) and additionally exhibits longer serum half-life. For example, IgG1 has a glycine (pI 5.97) at position 137, and IgG2 has a glutamic acid (pI 3.22); importing the glutamic acid will affect the pI of the resulting protein. As is described below, a number of amino acid substitutions are generally required to significantly affect the pI of the variant antibody. However, it should be noted as discussed below that even changes in IgG2 molecules allow for increased serum half-life.

In other embodiments, non-isotypic amino acid changes are made, either to reduce the overall charge state of the resulting protein (e.g., by changing a higher pI amino acid to a lower pI amino acid), or to allow accommodations in structure for stability, as is further described below.

In addition, by pI engineering both the heavy and light constant domains of a MBM comprising two half antibodies, significant changes in each half antibody can be seen. Having the pIs of the two half antibodies differ by at least 0.5 can allow separation by ion exchange chromatography or isoelectric focusing, or other methods sensitive to isoelectric point.

### 7.3.1.5.5. Calculating pI

The pI of a half antibody comprising an Fc region and a ABM or ABM chain can depend on the pI of the variant heavy chain constant domain and the pI of the total half antibody, including the variant heavy chain constant domain and ABM or ABM chain. Thus, in some embodiments, the change in pI is calculated on the basis of the variant heavy chain constant domain, using the chart in the FIG. 19 of US Pub. 2014/0370013. As discussed herein, which half antibody to engineer is generally decided by the inherent pI of the half antibodies. Alternatively, the pI of each half antibody can be compared.

### 7.3.1.5.6. pI Variants that Also Confer Better FcRn In Vivo Binding

In the case where a pI variant decreases the pI of an Fc region, it can have the added benefit of improving serum retention in vivo.

pI variant Fc regions are believed to provide longer half-lives to antigen binding molecules in vivo, because binding to FcRn at pH 6 in an endosome sequesters the Fc (Ghetie and Ward, 1997, Immunol Today. 18(12): 592-598). The endosomal compartment then recycles the Fc to the cell surface. Once the compartment opens to the extracellular space, the higher pH ˜7.4, induces the release of Fc back into the blood. In mice, Dall' Acqua et al. showed that Fc mutants with increased FcRn binding at pH 6 and pH 7.4 actually had reduced serum concentrations and the same half life as wild-type Fc (Dall' Acqua et al., 2002, J. Immunol. 169:5171-5180). The increased affinity of Fc for FcRn at pH 7.4 is thought to forbid the release of the Fc back into the blood. Therefore, the Fc mutations that will increase Fc's half-life in vivo will ideally increase FcRn binding at the lower pH while still allowing release of Fc at higher pH. The amino acid histidine changes its charge state in the pH range of 6.0 to 7.4. Therefore, it is not surprising to find His residues at important positions in the Fc/FcRn complex.

It has been suggested that antibodies with variable regions that have lower isoelectric points may also have longer serum half-lives (Igawa et al., 2010, PEDS. 23(5): 385-392). However, the mechanism of this is still poorly understood. Moreover, variable regions differ from antibody to antibody. Constant region variants with reduced pI and extended half-life would provide a more modular approach to improving the pharmacokinetic properties of MBMs, as described herein.

### 7.3.1.5.7. Polar Bridge

Heterodimerization of polypeptide chains of MBMs (e.g., BBMs) comprising an Fc domain can be increased by introducing modifications based on the “polar-bridging” rationale, which is to make residues at the binding interface of the two polypeptide chains to interact with residues of similar (or complimentary) physical property in the heterodimer configuration, while with residues of different physical property in the homodimer configuration. In particular, these modifications are designed so that, in the heterodimer formation, polar residues interact with polar residues, while hydrophobic residues interact with hydrophobic residues. In contrast, in the homodimer formation, residues are modified so that polar residues interact with hydrophobic residues. The favorable interactions in the heterodimer configuration and the unfavorable interactions in the homodimer configuration work together to make it more likely for Fc regions to form heterodimers than to form homodimers.

In an exemplary embodiment, the above modifications are generated at one or more positions of residues 364, 368, 399, 405, 409, and 411 of a CH3 domain.

In some embodiments, one or more modifications selected from the group consisting of S364L, T366V, L368Q, N399K, F405S, K409F and R411K are introduced into one of the two CH3 domains. One or more modifications selected from the group consisting of Y407F, K409Q and T411N can be introduced into the second CH3 domain.

In another embodiment, one or more modifications selected from the group consisting of S364L, T366V, L368Q, D399K, F405S, K409F and T411K are introduced into one CH3 domain, while one or more modifications selected the group consisting of from Y407F, K409Q and T411D are introduced into the second CH3 domain.

In one exemplary embodiment, the original residue of threonine at position 366 of one CH3 domain is replaced by valine, while the original residue of tyrosine at position 407 of the other CH3 domain is replaced by phenylalanine.

In another exemplary embodiment, the original residue of serine at position 364 of one CH3 domain is replaced by leucine, while the original residue of leucine at position 368 of the same CH3 domain is replaced by glutamine.

In yet another exemplary embodiment, the original residue of phenylalanine at position 405 of one CH3 domain is replaced by serine and the original residue of lysine at position 409 of this CH3 domain is replaced by phenylalanine, while the original residue of lysine at position 409 of the other CH3 domain is replaced by glutamine.

In yet another exemplary embodiment, the original residue of aspartic acid at position 399 of one CH3 domain is replaced by lysine, and the original residue of threonine at position 411 of the same CH3 domain is replaced by lysine, while the original residue of threonine at position 411 of the other CH3 domain is replaced by aspartic acid.

The amino acid replacements described herein can be introduced into the CH3 domains using techniques which are well known (see, e.g., McPherson, ed., 1991, Directed Mutagenesis: a Practical Approach; Adelman et al., 1983, DNA, 2:183). The polar bridge strategy is described in, for example, WO2006/106905, WO2009/089004 and K. Gunasekaran, et al. (2010) JBC, 285:19637-19646.

Additional polar bridge modifications are described in, for example, PCT publication no. WO2014/145806 (for example, FIG. 6 of WO2014/145806), PCT publication no. WO2014/110601, and PCT publication no. WO 2016/086186, WO 2016/086189, WO 2016/086196 and WO 2016/182751. An example of a polar bridge variant comprises a constant chain comprising a N208D, Q295E, N384D, Q418E and N421D modification.

In any of the embodiments described herein, the CH3 domains can be additionally modified to introduce a pair of cysteine residues as described in Section 7.3.1.3.

Additional strategies for enhancing heterodimerization are described in, for example, WO2016/105450, WO2016/086186, WO2016/086189, WO2016/086196, WO2016/141378, and WO2014/145806, and WO2014/110601. Any of the strategies can be employed in a MBM described herein.

### 7.3.1.6. Combination of Heterodimerization Variants and Other Fc Variants

As will be appreciated by a skilled artisan, all of the recited heterodimerization variants (including skew and/or pI variants) can be optionally and independently combined in any way, as long as the Fc regions of an Fc domain retain their ability to dimerize. In addition, all of these variants can be combined into any of the heterodimerization formats.

In the case of pI variants, while embodiments finding particular use are shown in the Table 5, other combinations can be generated, following the basic rule of altering the pI difference between two Fc regions in an Fc heterodimer to facilitate purification.

In addition, any of the heterodimerization variants, skew and pI, are also independently and optionally combined with Fc ablation variants, Fc variants, FcRn variants, as generally outlined herein.

In some embodiments, a particular combination of skew and pI variants that finds use in the present disclosure is T366S/L368A/Y407V:T366W (optionally including a bridging disulfide, T366S/L368A/Y407V/Y349C:T366W/S354C) with one Fc region comprising Q295E/N384D/Q418E/N481D and the other a positively charged scFv linker (when the format includes an scFv domain). As will be appreciated by a skilled artisan, the “knobs in holes” variants do not change pI, and thus can be used on either one of the Fc regions in an Fc heterodimer.

In some embodiments, first and second Fc regions that find use the present disclosure include the amino acid substitutions S364K/E357Q:L368D/K370S, where the first and/or second Fc region includes the ablation variant substitutions 233P/L234V/L235A/G236del/S267K, and the first and/or second Fc region comprises the pI variant substitutions N208D/Q295E/N384D/Q418E/N421D (pI_(−)_isosteric_A).

### 7.3.2. Hinge Regions

The MBMs (e.g., BBMs) can also comprise hinge regions, e.g., connecting an antigen-binding module to an Fc region. The hinge region can be a native or a modified hinge region. Hinge regions are typically found at the N-termini of Fc regions.

A native hinge region is the hinge region that would normally be found between Fab and Fc domains in a naturally occurring antibody. A modified hinge region is any hinge that differs in length and/or composition from the native hinge region. Such hinges can include hinge regions from other species, such as human, mouse, rat, rabbit, shark, pig, hamster, camel, llama or goat hinge regions. Other modified hinge regions can comprise a complete hinge region derived from an antibody of a different class or subclass from that of the heavy chain Fc region. Alternatively, the modified hinge region can comprise part of a natural hinge or a repeating unit in which each unit in the repeat is derived from a natural hinge region. In a further alternative, the natural hinge region can be altered by converting one or more cysteine or other residues into neutral residues, such as serine or alanine, or by converting suitably placed residues into cysteine residues. By such means the number of cysteine residues in the hinge region can be increased or decreased. This approach is described further in U.S. Pat. No. 5,677,425 by Bodmer et al., Altering the number of cysteine residues in a hinge region can, for example, facilitate assembly of light and heavy chains, or increase or decrease the stability of a MBM. Other modified hinge regions can be entirely synthetic and can be designed to possess desired properties such as length, cysteine composition and flexibility.

A number of modified hinge regions have been described for example, in U.S. Pat. No. 5,677,425, WO9915549, WO2005003170, WO2005003169, WO2005003170, WO9825971 and WO2005003171.

Examples of suitable hinge sequences are shown in Table 6.

TABLE 6
Hinge Sequences

| Hinge | Hinge | | SEQ ID |
|---|---|---|---|
| Name | Description | Hinge Sequence | NO: |
| H1 | Human IgA1 | VPSTPPTPSPSTPPTPSPS | 57 |
| H2 | Human IgA2 | VPPPPP | 58 |
| H3 | Human IgD | ESPKAQASSVPTAQPQAEGSLAKATTAPATTRN | 59 |
| | | TGRGGEEKKKEKEKEEQEERETKTP | |
| H4 | Human IgG1 | EPKSCDKTHTCPPCP | 60 |
| H5 | Human IgG2 | ERKCCVECPPCP | 61 |
| H6 | Human IgG3 | ELKTPLGDTTHTCPRCPEPKSCDTPPPCPRCPE | 62 |
| | | PKSCDTPPPCPRCPEPKSCDTPPPCPRCP | |
| H7 | Human IgG4 | ESKYGPPCPSCP | 63 |
| H8 | Human IgG4(P) | ESKYGPPCPPCP | 64 |
| H9 | Engineered v1 | CPPC | 55 |
| H10 | Engineered v2 | CPSC | 65 |
| H11 | Engineered v3 | CPRC | 66 |
| H12 | Engineered v4 | SPPC | 67 |
| H13 | Engineered v5 | CPPS | 68 |
| H14 | Engineered v6 | SPPS | 56 |
| H15 | Engineered v7 | DKTHTCAA | 69 |
| H16 | Engineered v8 | DKTHTCPPCPA | 70 |
| H17 | Engineered v9 | DKTHTCPPCPATCPPCPA | 71 |
| H18 | Engineered v10 | DKTHTCPPCPATCPPCPATCPPCPA | 72 |
| H19 | Engineered v11 | DKTHTCPPCPAGKPTLYNSLVMSDTAGTCY | 73 |
| H20 | Engineered v12 | DKTHTCPPCPAGKPTHVNVSVVMAEVDGTCY | 74 |
| H21 | Engineered v13 | DKTHTCCVECPPCPA | 75 |
| H22 | Engineered v14 | DKTHTCPRCPEPKSCDTPPPCPRCPA | 76 |
| H23 | Engineered v15 | DKTHTCPSCPA | 77 |

In one embodiment, the heavy chain Fc region possesses an intact hinge region at its N-terminus.

In one embodiment, the heavy chain Fc region and hinge region are derived from IgG4 and the hinge region comprises the modified sequence CPPC (SEQ ID NO: 55). The core hinge region of human IgG4 contains the sequence CPSC (SEQ ID NO: 65) compared to IgG1 which contains the sequence CPPC (SEQ ID NO: 55). The serine residue present in the IgG4 sequence leads to increased flexibility in this region, and therefore a proportion of molecules form disulfide bonds within the same protein chain (an intrachain disulfide) rather than bridging to the other heavy chain in the IgG molecule to form the interchain disulfide. (Angel et al., 1993, Mol Immunol 30(1):105-108). Changing the serine residue to a proline to give the same core sequence as IgG1 allows complete formation of inter-chain disulfides in the IgG4 hinge region, thus reducing heterogeneity in the purified product. This altered isotype is termed IgG4P.

### 7.3.3. ABM Linkers

In certain aspects, the present disclosure provides MBMs (e.g., BBMs) comprising at least three ABMs, where two or more components of an ABM (e.g., a VH and a VL of an scFv), two or more ABMs, or an ABM and a non-ABM domain (e.g., a dimerization domain such as an Fc region) are connected to one another by a peptide linker. Such linkers are referred to herein an “ABM linkers”, as opposed to the ADC linkers used to attach drugs to MBMs as described, for example, in Section 7.12.2.

A peptide linker can range from 2 amino acids to 60 or more amino acids, and in certain aspects a peptide linker ranges from 3 amino acids to 50 amino acids, from 4 to 30 amino acids, from 5 to 25 amino acids, from 10 to 25 amino acids or from 12 to 20 amino acids. In particular embodiments, a peptide linker is 2 amino acids, 3 amino acids, 4 amino acid, 5 amino acids, 6 amino acids, 7 amino acids, 8 amino acids, 9 amino acids, 10 amino acids, 11 amino acids, 12 amino acids, 13 amino acids, 14 amino acid, 15 amino acids, 16 amino acids, 17 amino acids, 18 amino acids, 19 amino acids, 20 amino acids, 21 amino acids, 22 amino acids, 23 amino acids, 24 amino acid, 25 amino acids, 26 amino acids, 27 amino acids, 28 amino acids, 29 amino acids, 30 amino acids, 31 amino acids, 32 amino acids, 33 amino acids, 34 amino acid, 35 amino acids, 36 amino acids, 37 amino acids, 38 amino acids, 39 amino acids, 40 amino acids, 41 amino acids, 42 amino acids, 43 amino acids, 44 amino acid, 45 amino acids, 46 amino acids, 47 amino acids, 48 amino acids, 49 amino acids, or 50 amino acids in length.

Charged and/or flexible linkers can be used.

Examples of flexible ABM linkers that can be used in the MBMs include those disclosed by Chen et al., 2013, Adv Drug Deliv Rev. 65(10):1357-1369 and Klein et al., 2014, Protein Engineering, Design & Selection 27(10):325-330. A particularly useful flexible linker is (GGGGS)n (also referred to as (G4S)n) (SEQ ID NO: 1318). In some embodiments, n is any number between 1 and 10, i.e., 1, 2, 3, 4, 5, 6, 7, 8, 9, and 10, or any range bounded by any two of the foregoing numbers, e.g., 1 to 5, 2 to 5, 3 to 6, 2 to 4, 1 to 4, and so on and so forth.

Other examples of suiTable 8BM linkers for use in the MBMs of the present disclosure are shown in Table 7 below:

TABLE 7
ABM Linker Sequences

| | | SEQ |
|---|---|---|
| Linker | | ID |
| Name | Linker Sequence | NO: |
| L1 | ADAAP | 79 |
| L2 | ADAAPTVSIFP | 80 |
| L3 | ADAAPTVSIFPP | 81 |
| L4 | AKTTAP | 82 |
| L5 | AKTTAPSVYPLAP | 83 |
| L6 | AKTTPKLEEGEFSEARV | 84 |
| L7 | AKTTPKLGG | 85 |
| L8 | AKTTPP | 86 |
| L9 | AKTTPPSVTPLAP | 87 |
| L10 | ASTKGP | 88 |
| L11 | ASTKGPSVFPLAP | 89 |
| L12 | ASTKGPSVFPLAPAST | 90 |
| | KGPSVFPLAP | |
| L13 | EGKSSGSGSESKST | 91 |
| L14 | GEGESGEGESGEGES | 92 |
| L15 | GEGESGEGESGEGESGEGES | 93 |
| L16 | GEGGSGEGGSGEGGS | 94 |
| L17 | GENKVEYAPALMALS | 95 |
| L18 | GGEGSGGEGSGGEGS | 96 |
| L19 | GGGESGGEGSGEGGS | 97 |
| L20 | GGGESGGGESGGGES | 98 |
| L21 | (GGGGS)ₙ (also referred | 99 |
| | to as (G4S)ₙ), | |
| | where n can be 1-10. | |
| L22 | GGGGSGGGGS | 100 |
| L23 | GGGGSGGGGSGGGGS | 53 |
| L24 | GGGGSGGGGSGGGGSGGGGS | 101 |
| L25 | GGGKSGGGKSGGGKS | 102 |
| L26 | GGGKSGGKGSGKGGS | 103 |
| L27 | GGKGSGGKGSGGKGS | 104 |
| L28 | GGSGG | 105 |
| L29 | GGSGGGGSG | 106 |
| L30 | GGSGGGGSGGGGS | 107 |
| L31 | GHEAAAVMQVQYPAS | 108 |
| L32 | GKGGSGKGGSGKGGS | 109 |
| L33 | GKGKSGKGKSGKGKS | 110 |
| L34 | GKGKSGKGKSGKGKSGKGKS | 111 |
| L35 | GKPGSGKPGSGKPGS | 112 |
| L36 | GKPGSGKPGSGKPGSGKPGS | 113 |
| L37 | GPAKELTPLKEAKVS | 114 |
| L38 | GSAGSAAGSGEF | 115 |
| L39 | IRPRAIGGSKPRVA | 116 |
| L40 | KESGSVSSEQLAQFRSLD | 117 |
| L41 | KTTPKLEEGEFSEAR | 118 |
| L42 | QPKAAP | 119 |
| L43 | QPKAAPSVTLFPP | 120 |
| L44 | RADAAAA(G4S)₄ | 121 |
| L45 | RADAAAAGGPGS | 122 |
| L46 | RADAAP | 123 |
| L47 | RADAAPTVS | 124 |
| L48 | SAKTTP | 125 |
| L49 | SAKTTPKLEEGEFSEARV | 126 |
| L50 | SAKTTPKLGG | 127 |
| L51 | STAGDTHLGGEDFD | 128 |
| L52 | TVAAP | 129 |
| L53 | TVAAPSVFIFPP | 130 |
| L54 | TVAAPSVFIFPPTVAAPSVFIFPP | 131 |
| L55 | GSTSGSGKPGSGEGSTKG | 132 |
| L56 | PRGASKSGSASQTGSAPGS | 133 |
| L57 | GTAAAGAGAAGGAAAGAAG | 134 |
| L58 | GTSGSSGSGSGGSGSGGGG | 135 |

In various aspects, the disclosure provides a MBM (e.g., a BBM) which comprises one or more ABM linkers. Each of the ABM linkers can be range from 2 amino acids to 60 amino acids in length, e.g., 4 to 30 amino acids, from 5 to 25 amino acids, from 10 to 25 amino acids or from 12 to 20 amino acids in length, optionally selected from Table 7 above. In particular embodiments, the MBM comprises two, three, four, five or six ABM linkers. The ABM linkers can be on one, two, three, four or even more polypeptide chains of the MBM.

### 7.4. Bispecific Binding Molecule Configurations

First and second MBMs can be BBMs. First and second MBMs that are BBMs are referred to herein as “first BBMs” and “second BBMs”, respectively. Exemplary BBM configurations are shown in FIG. 1. FIG. 1A shows the components of the BBM configurations shown in FIGS. 1B-1AH. The scFv, Fab, scFab, non-immunoglobulin based ABM, and Fc domains each can have the characteristics described for these components in Sections 7.2 and 7.3. The components of the BBM configurations shown in FIG. 1 can be associated with each other by any of the means described in Section 7.3 (e.g., by direct bonds, ABM linkers, disulfide bonds, Fc domains with modified with knob-in-hole interactions, etc.). The orientations and associations of the various components shown in FIG. 1 are merely exemplary; as will be appreciated by a skilled artisan, other orientations and associations can be suitable.

BBMs are not limited to the configurations shown in FIG. 1. Other configurations that can be used are known to those skilled in the art. See, e.g., WO 2014/145806; WO 2017/124002; Liu et al., 2017, Front Immunol. 8:38; Brinkmann & Kontermann, 2017, mAbs 9:2, 182-212; US 2016/0355600; Klein et al., 2016, MAbs 8(6):1010-20; and US 2017/0145116.

### 7.4.1. Exemplary Bivalent BBMs

First and second BBMs can be bivalent. For example, a first BBM can have a single ABM1 and a single ABM2 or ABM3, and a second BBM can have a single ABM4 and a single ABM5 or ABM6.

Exemplary bivalent BBM configurations are shown in FIGS. 1B-1F.

As depicted in FIGS. 1B-1D, a BBM can comprise two half antibodies, one comprising one ABM and the other comprising one ABM, the two halves paired through an Fc domain.

In the embodiment of FIG. 1B, the first (or left) half antibody comprises a Fab and an Fc region, and the second (or right) half antibody comprises a Fab and an Fc region. The first and second half antibodies are associated through the Fc regions forming an Fc domain.

In the embodiment of FIG. 1C, the first (or left) half antibody comprises a Fab and an Fc region, and the second (or right) half antibody comprises a scFv and an Fc region. The first and second half antibodies are associated through the Fc regions forming an Fc domain.

In the embodiment of FIG. 1D, the first (or left) half antibody comprises an scFv and an Fc region, and the second (or right) half antibody comprises an scFv and an Fc region. The first and second half antibodies are associated through the Fc regions forming an Fc domain.

As depicted in FIGS. 1E-1F, a bivalent BBM can comprise two ABMs attached to one Fc region of an Fc domain.

In the embodiment of FIG. 1E, the BBM comprises a Fab, a scFv and an Fc domain, where the scFv is located between the Fab and the Fc domain.

In the embodiment of FIG. 1F, (the “one-arm scFv-mAb” configuration) BBM comprises a Fab, a scFv and an Fc domain, where the Fab is located between the scFv and the Fc domain.

In the configuration shown in FIGS. 1B-1F, when the BBM is a first BBM, each of X and Y represent either (i) ABM1 or (ii) ABM2 or ABM3, provided that the BBM comprises one ABM1 and one ABM2 or ABM3. When the BBM is a second BBM, each of X and Y represent either (i) ABM4 or (ii) ABM5 or ABM6, provided that the BBM comprises one ABM4 and one ABM5 or ABM6. Accordingly, the present disclosure provides bivalent BBMs as shown in any one of FIGS. 1B through 1F, where X is an ABM1 and Y is an ABM2 or ABM3 and provides bivalent BBMs as shown in any one of FIGS. 1B through 1F, where X is an ABM4 and Y is an ABM5 or ABM6 (such configurations of ABMs designated as “B1” for convenience). The present disclosure also provides bivalent BBMs as shown in any one of FIGS. 1B through 1F, where X is an ABM2 or ABM3 and Y is an ABM1, and provides bivalent BBMs as shown in any one of FIGS. 1B through 1F, where X is ABM5 or ABM6 and Y is ABM4 (such configurations of ABMs designated as “B2” for convenience).

### 7.4.2. Exemplary Trivalent BBMs

The BBMs can be trivalent, i.e., they have three antigen-binding domains, one or two of which binds a first target antigen and one or two of which binds a second target antigen. For example, a first BBM that is trivalent can have a single ABM1 and two ABM2s or ABM3s, or two ABM2s or ABM3s and a single ABM1. Likewise, a second BBM that is trivalent can have a single ABM4 and two ABM5s or ABM6s, or two ABM4s and a single ABM5 or ABM6.

Exemplary trivalent BBM configurations are shown in FIGS. 1G-1Z.

As depicted in FIGS. 1G-1N, 1Q-1W, 1Y-1Z a BBM can comprise two half antibodies, one comprising two ABMs and the other comprising one ABM, the two halves paired through an Fc domain.

In the embodiment of FIG. 1G, the first (or left) half antibody comprises Fab and an Fc region, and the second (or right) half antibody comprises a scFv, a Fab, and an Fc region. The first and second half antibodies are associated through the Fc regions forming an Fc domain.

In the embodiment of FIG. 1H, the first (or left) half antibody comprises a Fab and an Fc region, and the second (or right) half antibody comprises a Fab, an scFv, and an Fc region. The first and second half antibodies are associated through the Fc regions forming an Fc domain.

In the embodiment of FIG. 1I, the first (or left) half antibody comprises an scFv and an Fc region, and the second (or right) half antibody comprises two Fabs and an Fc region. The first and second half antibodies are associated through the Fc regions forming an Fc domain.

In the embodiment of FIG. 1J, the first (or left) half antibody comprises two Fav and an Fc region, and the second (or right) half antibody comprises a Fab and an Fc region. The first and second half antibodies are associated through the Fc regions forming an Fc domain.

In the embodiment of FIG. 1K, the first (or left) half antibody comprises an scFv and an Fc region, and the second (or right) half antibody comprises two scFvs and an Fc region. The first and second half antibodies are associated through the Fc regions forming an Fc domain.

In the embodiment of FIG. 1L, the first (or left) half antibody comprises an scFv and an Fc region, and the second (or right) half antibody comprises an scFv, a Fab, and an Fc region. The first and second half antibodies are associated through the Fc regions forming an Fc domain.

In the embodiment of FIG. 1M, the first (or left) half antibody comprises a scFv and an Fc region, and the second (or right) half antibody comprises a Fab, a scFv and an Fc region. The first and second half antibodies are associated through the Fc regions forming an Fc domain.

In the embodiment of FIG. 1N, the first (or left) half antibody comprises a diabody-type binding domain and an Fc region, and the second (or right) half antibody comprises a Fab and an Fc region. The first and second half antibodies are associated through the Fc regions forming an Fc domain.

In the embodiment of FIG. 1Q, the first (or left) half antibody comprises a Fab and an Fc region, and the second (or right) half antibody comprises a Fab, an Fc region, and an scFv. The first and second half antibodies are associated through the Fc regions forming an Fc domain.

In the embodiment of FIG. 1R, the first (or left) half antibody comprises a scFv and an Fc region, and the second (or right) half antibody comprises a Fab, an Fc region, and an scFv. The first and second half antibodies are associated through the Fc regions forming an Fc domain.

In the embodiment of FIG. 1S, the first (or left) half antibody comprises an scFv and an Fc region, and the second (or right) half antibody comprises an scFv, an Fc region, and a second scFv. The first and second half antibodies are associated through the Fc regions forming an Fc domain.

In the embodiment of FIG. 1T, the first (or left) half antibody comprises an scFv, an Fc region, and a Fab, and the second (or right) half antibody comprises a Fab and an Fc region. The first and second half antibodies are associated through the Fc regions forming an Fc domain.

In the embodiment of FIG. 1U, the first (or left) half antibody comprises two Fab and an Fc region, and the second (or right) half antibody comprises a non-immunoglobulin based ABM and an Fc region. The first and second half antibodies are associated through the Fc regions forming an Fc domain.

In the embodiment of FIG. 1V, the first (or left) half antibody comprises a Fab, an scFv, and an Fc region, and the second (or right) half antibody comprises a non-immunoglobulin based ABM and an Fc region. The first and second half antibodies are associated through the Fc regions forming an Fc domain.

In the embodiment of FIG. 1W, the first (or left) half antibody comprises a Fab and an Fc region, and the second (or right) half antibody comprises a scFv, a non-immunoglobulin based ABM, and an Fc region. The first and second half antibodies are associated through the Fc regions forming an Fc domain.

In the embodiment of FIG. 1Y, the first (or left) half antibody comprises an scFv and an Fc region, and the second (or right) half antibody comprises a Fab, an scFv and an Fc region. The first and second half antibodies are associated through the Fc regions forming an Fc domain.

In the embodiment of FIG. 1Z, the first (or left) half antibody comprises a Fab, an Fc region, and a scFab, and the second (or right) half antibody comprises a Fab and an Fc region. The first and second half antibodies are associated through the Fc regions forming an Fc domain.

Alternatively, as depicted in FIGS. 1O and 1P, trivalent a BBM can comprise two half antibodies, each comprising one complete ABM (a Fab in FIGS. 1O and 1P) and a portion of another ABM (one a VH, the other a VL). The two half antibodies are paired through an Fc domain, whereupon the VH and the VL associate to form a complete antigen-binding Fv domain.

The BBM can be a single chain, as shown in FIG. 1X. The BBM of FIG. 1X comprises three scFv domains connected through linkers.

In the configuration shown in FIGS. 1G-1Z, each of X, Y and A represent, in the case of a first BBM, either (i) an ABM1 or (ii) ABM2 or ABM3, provided that the BBM comprises (i) at least one ABM1 and (ii) at least one ABM2 or ABM3, and in the case of a second MBM, either (i) an ABM4 or (ii) ABM5 or ABM6, provided the BBM comprises at least one ABM4 and at least one ABM5 or ABM6. Thus, the trivalent BBMs will include one or two ABM1s and one or two ABM2s or ABM3s in the case of a first BBM and one or two ABM4s and one or two ABM5s or ABM6s in the case of a second BBM. In some embodiments, a trivalent first BBM comprises two ABM1s and one ABM2. In other embodiments, a trivalent first BBM comprises one ABM1 and two ABM2s. In some embodiments, a trivalent first BBM comprises two ABM1s and one ABM3. In other embodiments, a trivalent first BBM comprises one ABM1 and two ABM3s. In some embodiments, a trivalent second BBM comprises two ABM4s and one ABM5. In other embodiments, a trivalent second BBM comprises one ABM4 and two ABM5s. In some embodiments, a trivalent second BBM comprises two ABM4s and one ABM6. In other embodiments, a trivalent second BBM comprises one ABM4 and two ABM6s.

Accordingly, in the present disclosure provides trivalent BBMs as shown in any one of FIGS. 1G through 1Z, where X is an ABM1, Y is an ABM1 and A is an ABM2 or ABM3, and provides trivalent BBMs as shown in any one of FIGS. 1G through 1Z, where X is an ABM4, Y is an ABM4 and A is an ABM5 or ABM6 (such configurations of ABMs designated as “T1” for convenience).

The disclosure further provides trivalent BBMs as shown in any one of FIGS. 1G through 1Z, where X is an ABM1, Y is an ABM2 or ABM3 and A is an ABM1, and provides trivalent BBMs as shown in any one of FIGS. 1G through 1Z, where X is an ABM4, Y is an ABM5 or ABM6 and A is an ABM4 (such configurations of ABMs designated as “T2” for convenience).

The disclosure further provides trivalent BBMs as shown in any one of FIGS. 1G through 1Z, where X is an ABM2 or ABM3, Y is an ABM1 and A is an ABM1, and provides trivalent BBMs as shown in any one of FIGS. 1G through 1Z, where X is an ABM5 or ABM6, Y is an ABM4 and A is an ABM4 (such configurations of ABMs designated as “T3” for convenience).

The disclosure further provides trivalent BBMs as shown in any one of FIGS. 1G through 1Z, where X is an ABM1, Y is an ABM2 or ABM3 and A is an ABM2 or ABM3, and provides trivalent BBMs as shown in any one of FIGS. 1G through 1Z, where X is an ABM4, Y is an ABM5 or ABM6 and A is an ABM5 or ABM6 (such configurations of ABMs designated as “T4” for convenience).

The disclosure further provides trivalent BBMs as shown in any one of FIGS. 1G through 1Z, where X is an ABM2 or ABM3, Y is an ABM1 and A is an ABM2 or ABM3, and provides trivalent BBMs as shown in any one of FIGS. 1G through 1Z, where X is an ABM5 or ABM6, Y is an ABM4 and A is an ABM5 or ABM6 (such configurations of ABMs designated as “T5” for convenience).

The disclosure further provides trivalent BBMs as shown in any one of FIGS. 1G through 1Z, where X is an ABM2 or ABM3, Y is an ABM2 or ABM3 and A is an ABM1, and provides trivalent BBMs as shown in any one of FIGS. 1G through 1Z, where X is an ABM5 or ABM6, Y is an ABM5 or ABM6 and A is an ABM4 (such configurations of ABMs designated as “T6” for convenience).

### 7.4.3. Exemplary Tetravalent BBMs

The BBMs can be tetravalent, i.e., they have four antigen-binding domains, one, two, or three of which binds a first target antigen and one, two, or three of which binds a second target antigen. Exemplary tetravalent BBM configurations are shown in FIGS. 1AA-1AH.

As depicted in FIGS. 1AA-1AH, a tetravalent BBM can comprise two half antibodies, each comprising two complete ABMs, the two halves paired through an Fc domain.

In the embodiment of FIG. 1AA, the first (or left) half antibody comprises a Fab, an Fc region, and an scFv, and the second (or right) half antibody comprises a Fab, an Fc region, and an scFv. The first and second half antibodies are associated through the Fc regions forming an Fc domain.

In the embodiment of FIG. 1AB, the first (or left) half antibody comprises a Fab, an scFv, and an Fc region, and the second (or right) half antibody comprises a Fab, an scFv, and an Fc region. The first and second half antibodies are associated through the Fc regions forming an Fc domain.

In the embodiment of FIG. 1AC, the first (or left) half antibody comprises an scFv, a Fab, and an Fc region, and the second (or right) half antibody comprises an scFv, a Fab, and an Fc region. The first and second half antibodies are associated through the Fc regions forming an Fc domain.

In the embodiment of FIG. 1AD, the first (or left) half antibody comprises a Fab, an Fc region, and a second Fab, and the second (or right) half antibody comprises a Fab, an Fc region, and a second Fab. The first and second half antibodies are associated through the Fc regions forming an Fc domain.

In the embodiment of FIG. 1AE, the first (or left) half antibody comprises an scFv, a second scFv, and an Fc region, and the second (or right) half antibody comprises an scFv, a second scFv, and an Fc region. The first and second half antibodies are associated through the Fc regions forming an Fc domain.

In the embodiment of FIG. 1AF, the first (or left) half antibody comprises a Fab, an scFv, and an Fc region, and the second (or right) half antibody comprises a Fab, an scFv, and an Fc region. The first and second half antibodies are associated through the Fc regions forming an Fc domain.

In the embodiment of FIG. 1AG, the first (or left) half antibody comprises a Fab, an Fc region, and an scFv, and the second (or right) half antibody comprises a scFv, an Fc region, and a Fab. The first and second half antibodies are associated through the Fc regions forming an Fc domain.

In the embodiment of FIG. 1AH, the first (or left) half antibody comprises a scFv, an Fc region, and an Fab, and the second (or right) half antibody comprises a scFv, an Fc region, and a Fab. The first and second half antibodies are associated through the Fc regions forming an Fc domain.

In the configuration shown in FIGS. 1AA-1AH, for a first BBM, each of X, Y, A, and B represent (i) ABM1 or (ii) ABM2 or ABM3, although not necessarily in that order, and provided that the BBM comprises at least one ABM1 and at least one ABM2 or ABM3. Thus, the tetravalent first BBMs will include one, two, or three ABM1s and one, two, or three ABM2s or ABM3s. In some embodiments, a tetravalent BBM comprises three ABM1s and one ABM2 or ABM3. In other embodiments, a tetravalent BBM comprises two ABM1s and two ABM2s or ABM3s. In yet other embodiments, a tetravalent BBM comprises one ABM1 and three ABM2s or ABM3s. Likewise, for a second BBM, each of X, Y, A, and B represent (i) ABM4 or (ii) ABM5 or ABM6, although not necessarily in that order, and provided that the BBM comprises at least one ABM4 and at least one ABM5 or ABM6. Thus, the tetravalent second BBMs will include one, two, or three ABM4s and one, two, or three ABM5s of ABM6s. In some embodiments, a tetravalent BBM comprises three ABM4s and one ABM5 or ABM6. In other embodiments, a tetravalent BBM comprises two ABM4s and two ABM5s of ABM6s. In yet other embodiments, a tetravalent BBM comprises one ABM4 and three ABM5s or ABM6s.

Accordingly, the present disclosure provides tetravalent BBMs as shown in any one of FIGS. 1AA-1AH, where X is an ABM1 and each of Y, A, and B are ABM2s or ABM3s, and provides tetravalent BBMs as shown in any one of FIGS. 1AA-1AH, where X is an ABM4 and each of Y, A, and B are ABM5s or ABM6s (such configurations of ABMs designated as “Tv 1” for convenience).

The disclosure further provides tetravalent BBMs as shown in any one of FIGS. 1AA-1AH, where Y is an ABM1 and each of X, A, and B are ABM2s or ABM3s, and provides tetravalent BBMs as shown in any one of FIGS. 1AA-1AH, where Y is an ABM4 and each of X, A, and B are ABM5s or ABM6s, (such configurations of ABMs designated as “Tv 2” for convenience).

The disclosure further provides tetravalent BBMs as shown in any one of FIGS. 1AA-1AH, where A is an ABM1 and each of X, Y, and B are ABM2s or ABM3s, and provides tetravalent BBMs as shown in any one of FIGS. 1AA-1AH, where A is an ABM4 and each of X, Y, and B are ABM5s or ABM6s (such configurations of ABMs designated as “Tv 3” for convenience).

The disclosure further provides tetravalent BBMs as shown in any one of FIGS. 1AA-1AH, where B is an ABM1 and each of X, Y, and A are ABM2s or ABM3s, and provides tetravalent BBMs as shown in any one of FIGS. 1AA-1AH, where B is an ABM4 and each of X, Y, and A are ABM5s or ABM6s (such configurations of ABMs designated as “Tv 4” for convenience).

The disclosure further provides tetravalent BBMs as shown in any one of FIGS. 1AA-1AH, where X and Y are both ABM and both of A and B are ABM2s or ABM3s, and provides tetravalent BBMs as shown in any one of FIGS. 1AA-1AH, where X and Y are both ABM4s and both of A and B are ABM5s or ABM6s (such configurations of ABMs designated as “Tv 5” for convenience).

The disclosure further provides tetravalent BBMs as shown in any one of FIGS. 1AA-1AH, where X and A are both ABM1s and both of Y and B are ABM2s or ABM3s, and provides tetravalent BBMs as shown in any one of FIGS. 1AA-1AH, where X and A are both ABM4s and both of Y and B are ABM5s or ABM6s (such configurations of ABMs designated as “Tv 6” for convenience).

The disclosure further provides tetravalent BBMs as shown in any one of FIGS. 1AA-1AH, where X and B are both ABM1s and both of Y and A are ABM2s or ABM3s, and provides tetravalent BBMs as shown in any one of FIGS. 1AA-1AH, where X and B are both ABM4s and both of Y and A are ABM5s or ABM6s (such configurations of ABMs designated as “Tv 7” for convenience).

The disclosure further provides tetravalent BBMs as shown in any one of FIGS. 1AA-1AH, where Y and A are both ABM1s and both of X and B are ABM2s or ABM3s, and provides tetravalent BBMs as shown in any one of FIGS. 1AA-1AH, where Y and A are both ABM4s and both of X and B are ABM5s or ABM6s (such configurations of ABMs designated as “Tv 8” for convenience).

The disclosure further provides tetravalent BBMs as shown in any one of FIGS. 1AA-1AH, where Y and B are both ABM1s and both of X and A are ABM2s or ABM3s, and provides tetravalent BBMs as shown in any one of FIGS. 1AA-1AH, where Y and B are both ABM4s and both of X and A are ABM5s or ABM6s (such configurations of ABMs designated as “Tv 9” for convenience).

The disclosure further provides tetravalent BBMs as shown in any one of FIGS. 1AA-1AH, where A and B are both ABM1s and both of X and Y are ABM2s or ABM3s, and provides tetravalent BBMs as shown in any one of FIGS. 1AA-1AH, where A and B are both ABM4s and both of X and Y are ABM5s or ABM6s (such configurations of ABMs designated as “Tv 10” for convenience).

The disclosure further provides tetravalent BBMs as shown in any one of FIGS. 1AA-1AH, where each of X, Y, and A is an ABM1 and B is an ABM2 or ABM3, and provides tetravalent BBMs as shown in any one of FIGS. 1AA-1AH, where each of X, Y, and A is an ABM4 and B is an ABM5 or ABM6 (such configurations of ABMs designated as “Tv 11” for convenience).

The disclosure further provides tetravalent BBMs as shown in any one of FIGS. 1AA-1AH, where each of X, Y, and B is an ABM1 and A is an ABM2 or ABM3, and provides tetravalent BBMs as shown in any one of FIGS. 1AA-1AH, where each of X, Y, and B is an ABM4 and A is an ABM5 or ABM6 (such configurations of ABMs designated as “Tv 12” for convenience).

The disclosure further provides tetravalent BBMs as shown in any one of FIGS. 1AA-1AH, where each of X, A, and B is an ABM1 and Y is an ABM2 or ABM3, and provides tetravalent BBMs as shown in any one of FIGS. 1AA-1AH, where each of X, A, and B is an ABM4 and Y is an ABM5 or ABM6 (such configurations of ABMs designated as “Tv 13” for convenience).

The disclosure further provides tetravalent BBMs as shown in any one of FIGS. 1AA-1AH, where each of Y, A, and B is an ABM1 and X is an ABM2 or ABM3, and provides tetravalent BBMs as shown in any one of FIGS. 1AA-1AH, where each of Y, A, and B is an ABM4 and X is an ABM5 or ABM6 (such configurations of ABMs designated as “Tv 14” for convenience).

7.5. Trispecific Binding Molecule Configurations

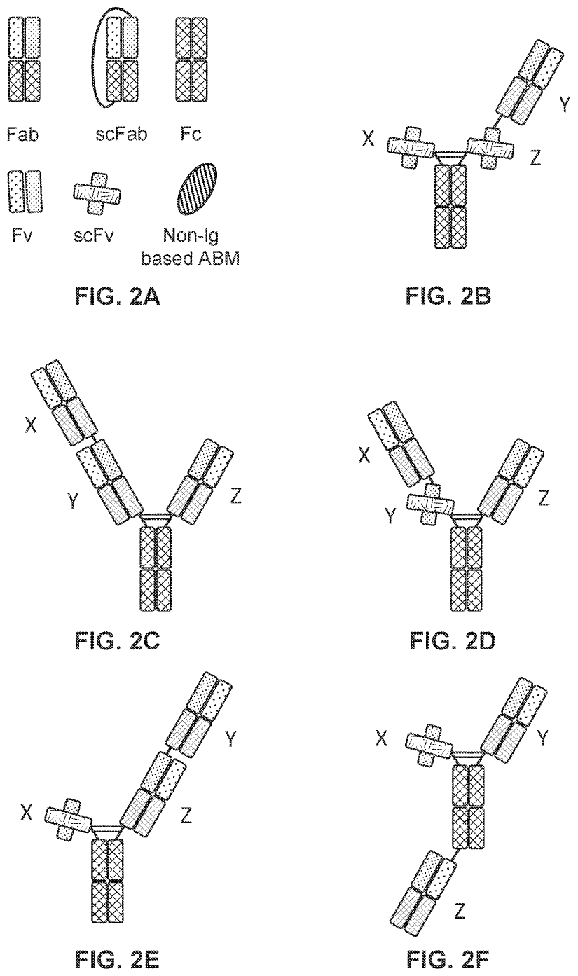

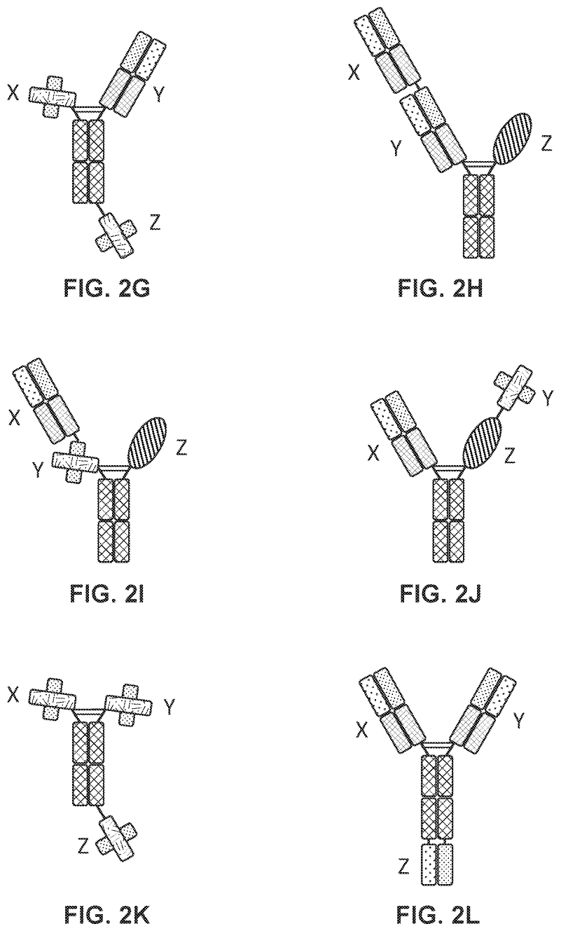

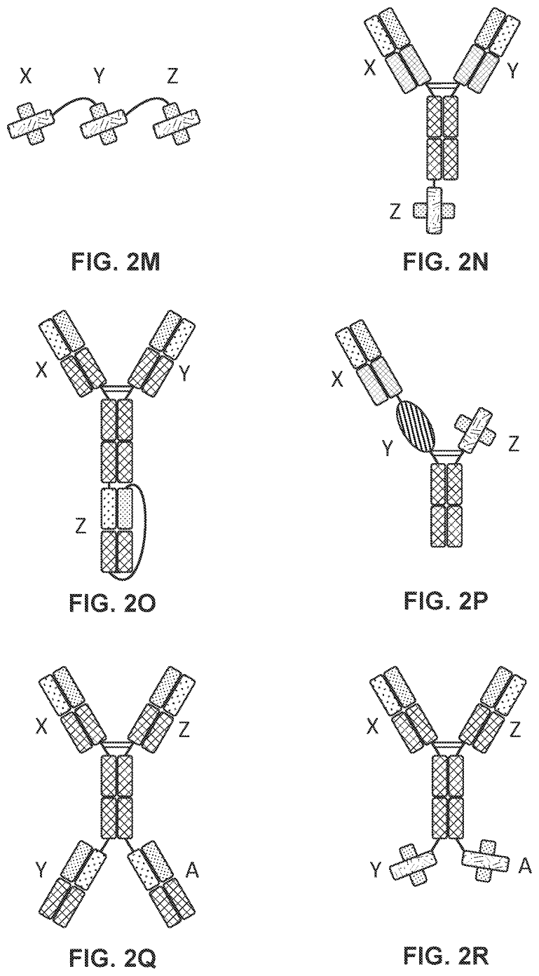

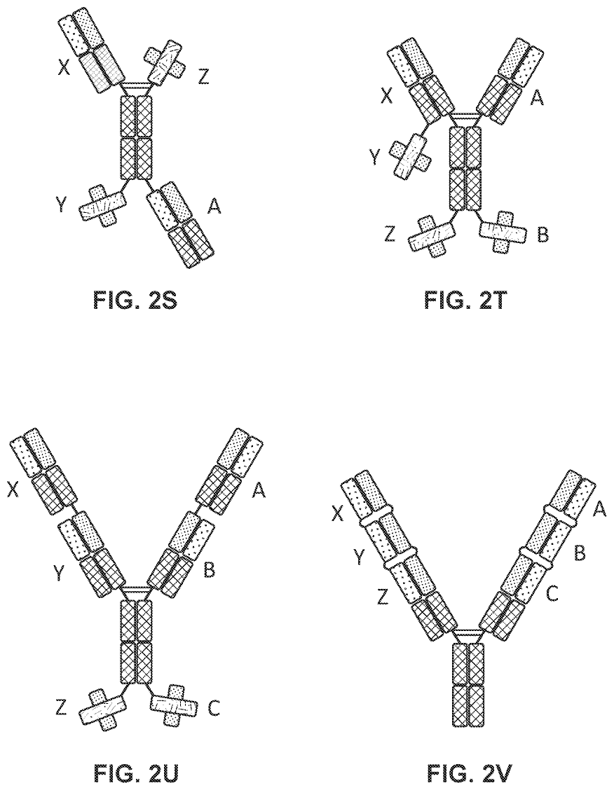

First and second MBMs can be TBMs. First and second MBMs that are TBMs are referred to herein as “first TBMs” and “second TBMs”, respectively. Exemplary TBM configurations are shown in FIG. 2. FIG. 2A shows the components of the TBM configurations shown in FIGS. 2B-2V. The scFv, Fab, non-immunoglobulin based ABM, and Fc each can have the characteristics described for these components in Sections 7.2 and 7.3. The components of the TBM configurations shown in FIG. 2 can be associated with each other by any of the means described in Section 7.3 (e.g., by direct bonds, ABM linkers, disulfide bonds, Fc domains with modified with knob in hole interactions, etc.). The orientations and associations of the various components shown in FIG. 2 are merely exemplary; as will be appreciated by a skilled artisan, other orientations and associations can be suitable (e.g., as described in Sections 7.2 and 7.3).

TBMs are not limited to the configurations shown in FIG. 2. Other configurations that can be used are known to those skilled in the art. See, e.g., WO 2014/145806; WO 2017/124002; Liu et al., 2017, Front Immunol. 8:38; Brinkmann & Kontermann, 2017, mAbs 9:2, 182-212; US 2016/0355600; Klein et al., 2016, MAbs 8(6):1010-20; and US 2017/0145116.

### 7.5.1. Exemplary Trivalent TBMs

The TBMs of the disclosure can be trivalent, e.g., they can have three antigen-binding modules, one of which binds a first target antigen, one of which binds a second target antigen, and one of which binds a third target antigen.

Exemplary trivalent TBM configurations are shown in FIGS. 2B through 2P. In the TBMs of the disclosure, a non-immunoglobulin-based domain can substitute for a Fab and/or scFv in any of the configurations illustrated.

As depicted in FIGS. 2B-2K and 2N-2P, a TBM can comprise two half antibodies, one comprising two ABMs and the other comprising one ABM, the two halves paired through an Fc domain.

In the embodiment of FIG. 2B, the first (or left) half antibody comprises an scFv and an Fc region, and the second (or right) half antibody comprises a Fab, an scFv and an Fc region. The first and second half antibodies are associated through the Fc regions forming an Fc domain.

In the embodiment of FIG. 2C, the first (or left) half antibody comprises two Fab and an Fc region, and the second (or right) half antibody comprises a Fab and an Fc region. The first and second half antibodies are associated through the Fc regions forming an Fc domain.

In the embodiment of FIG. 2D, the first (or left) half antibody comprises a Fab, an scFv and an Fc region, and the second (or right) half antibody comprises a Fab and an Fc region. The first and second half antibodies are associated through the Fc regions forming an Fc domain.

In the embodiment of FIG. 2E, the first (or left) half antibody comprises an scFv and an Fc region, and the second (or right) half antibody comprises two Fab and an Fc region. The first and second half antibodies are associated through the Fc regions forming an Fc domain.

In the embodiment of FIG. 2F, the first (or left) half antibody comprises an scFv, an Fc region, and a Fab, and the second (or right) half antibody comprises a Fab and an Fc region. The first and second half antibodies are associated through the Fc regions forming an Fc domain.

In the embodiment of FIG. 2G, the first (or left) half antibody comprises an scFv and an Fc region, and the second (or right) half antibody comprises a Fab an Fc region, and an scFV. The first and second half antibodies are associated through the Fc regions forming an Fc domain.

In the embodiment of FIG. 2H, the first (or left) half antibody comprises two Fab and an Fc region, and the second (or right) half antibody comprises a non-immunoglobulin based ABM and an Fc region. The first and second half antibodies are associated through the Fc regions forming an Fc domain.

In the embodiment of FIG. 2I, the first (or left) half antibody comprises a Fab, an scFv, and an Fc region, and the second (or right) half antibody comprises a non-immunoglobulin based ABM and an Fc region. The first and second half antibodies are associated through the Fc regions forming an Fc domain.

In the embodiment of FIG. 2J, the first (or left) half antibody comprises a Fab and an Fc region, and the second (or right) half antibody comprises an scFv, a non-immunoglobulin based ABM and an Fc region. The first and second half antibodies are associated through the Fc regions forming an Fc domain.

In the embodiment of FIG. 2K, the first (or left) half antibody comprises an scFv and an Fc region, and the second (or right) half antibody comprises an scFv, an Fc region, and a second scFv. The first and second half antibodies are associated through the Fc regions forming an Fc domain.

In the embodiment of FIG. 2N, the first (or left) half antibody comprises a Fab, an Fc region, and an scFv, and the second (or right) half antibody comprises a Fab, and an Fc region. The first and second half antibodies are associated through the Fc regions forming an Fc domain.

In the embodiment of FIG. 2O, the first (or left) half antibody comprises a Fab, an Fc region, and a scFab, and the second (or right) half antibody comprises a Fab and an Fc region. The first and second half antibodies are associated through the Fc regions forming an Fc domain.

In the embodiment of FIG. 2P, the first (or left) half antibody comprises a Fab, a non-immunoglobulin based ABM, and an Fc region, and the second (or right) half antibody comprises a scFv and an Fc region. The first and second half antibodies are associated through the Fc regions forming an Fc domain.

Alternatively, as depicted in FIG. 2L, trivalent a TBM can comprise two half antibodies, each comprising one complete ABM and a portion of another ABM (one a VH, the other a VL). The two half antibodies are paired through an Fc domain, whereupon the VH and the VL associate to form a complete antigen-binding Fv domain.

The TBM can be a single chain, as shown in FIG. 2M. The TBM of FIG. 2M comprises three scFv domains connected through linkers.

In each of the configurations shown in FIGS. 2B-2P, each of the domains designated X, Y, and Z can represent an ABM1, ABM2, or ABM3 for a first TBM, although not necessarily in that order, and can represent an ABM4, ABM5, or ABM6 for a second TBM, although not necessarily in that order. In other words, for a first TBM, X can be ABM1, ABM2, or ABM3, Y can be ABM1, ABM2, or ABM3, and Z can be ABM1, ABM2, or ABM3, provided that the TBM comprises one ABM1, one ABM2, and one ABM3. Likewise, for a second TBM, X can be ABM4, ABM5, or ABM6, Y can be ABM4, ABM5, or ABM6, and Z can be ABM4, ABM5, or ABM6, provided that the TBM comprises one ABM1, one ABM2, and one ABM3

Accordingly, in the present disclosure provides a trivalent TBM as shown in any one of FIGS. 2B through 2P, where X is an ABM1, Y is an ABM3 and Z is an ABM2, and provides a trivalent TBM as shown in any one of FIGS. 2B through 2P, where X is an ABM4, Y is an ABM6 and Z is an ABM5 (this configuration of ABMs designated as “T1” for convenience).

The present disclosure also provides a trivalent TBM as shown in any one of FIGS. 2B through 2P, where X is an ABM1, Y is an ABM2, and Z is an ABM3, and provides a trivalent TBM as shown in any one of FIGS. 2B through 2P, where X is an ABM4, Y is an ABM5, and Z is an ABM6 (this configuration of ABMs designated as “T2” for convenience).

The present disclosure further provides a trivalent TBM as shown in any one of FIGS. 2B through 2P, where X is an ABM3, Y is an ABM1, and Z is an ABM2 and provides a trivalent TBM as shown in any one of FIGS. 2B through 2P, where X is an ABM6, Y is an ABM4, and Z is an ABM5 (this configuration of ABMs designated as “T3” for convenience).

The present disclosure yet further provides a trivalent TBM as shown in any one of FIGS. 2B through 2P, where X is an ABM3, Y is an ABM2, and Z is an ABM1, and provides a trivalent TBM as shown in any one of FIGS. 2B through 2P, where X is an ABM6, Y is an ABM5, and Z is an ABM4 (this configuration of ABMs designated as “T4” for convenience).

The present disclosure yet further provides a trivalent TBM as shown in any one of FIGS. 2B through 2P, where X is an ABM2, Y is an ABM1, and Z is an ABM3, and provides a trivalent TBM as shown in any one of FIGS. 2B through 2P, where X is an ABM5, Y is an ABM4, and Z is an ABM6 (this configuration of ABMs designated as “T5” for convenience).

The present disclosure yet further provides a trivalent TBM as shown in any one of FIGS. 2B through 2P, where X is an ABM2, Y is an ABM3, and Z is an ABM1, and provides a trivalent TBM as shown in any one of FIGS. 2B through 2P, where X is an ABM5, Y is an ABM6, and Z is an ABM4 (this configuration of ABMs designated as “T6” for convenience).

### 7.5.2. Exemplary Tetravalent TBMs

The TBMs of the disclosure can be tetravalent, e.g., they can have four antigen-binding modules, one or two of which finds a first target antigen, one or two of which binds a second target antigen, and one or two of which binds a third target antigen.

Exemplary tetravalent TBM configurations are shown in FIGS. 2Q-2S. In the TBMs of the disclosure, a non-immunoglobulin-based domain can substitute for a Fab and/or scFv in any of the configurations illustrated.

As depicted in FIGS. 2Q-2S, a tetravalent TBM can comprise two half antibodies, each comprising two complete ABMs, the two halves paired through an Fc domain.

In the embodiment of FIG. 2Q, the first (or left) half antibody comprises a Fab, an Fc region, and a second Fab, and the second (or right) half antibody comprises a Fab, an Fc region, and a second Fab. The first and second half antibodies are associated through the Fc regions forming an Fc domain.

In the embodiment of FIG. 2R, the first (or left) half antibody comprises a Fab, an Fc region, and an scFv, and the second (or right) half antibody comprises a Fab, an Fc region, and an scFv. The first and second half antibodies are associated through the Fc regions forming an Fc domain.

In the embodiment of FIG. 2S, the first (or left) half antibody comprises a Fab, an Fc region, and an scFv, and the second (or right) half antibody comprises an scFv, an Fc region, and a Fab. The first and second half antibodies are associated through the Fc regions forming an Fc domain.

In the configuration shown in FIGS. 2Q-2S, for a first TBM having an ABM1, an ABM2, and an ABM3, each of X, Y, Z, and A represent an ABM1, an ABM2, or an ABM3, although not necessarily in that order, and provided that the TBM comprises at least one ABM1, at least one ABM2, and at least one ABM3. In some cases, a tetravalent first TBM has two ABM1s, two ABM2s, or two ABM3s. Likewise, for a second TBM having an ABM4, an ABM5, and an ABM6, each of X, Y, Z, and A represent an ABM4, an ABM5, or an ABM6, although not necessarily in that order, and provided that the TBM comprises at least one ABM4, at least one ABM5, and at least one ABM6. In some cases, a tetravalent TBM has two ABM4s, two ABM5s, or two ABM6s.

Accordingly, the present disclosure provides tetravalent TBMs as shown in any one of FIGS. 2Q-2S, where X, Y, Z, and A are ABM1s, ABM2s, and ABM3s (for a first TBM) or ABM4s, ABM5s, and ABM6s (for a second TBM), as shown in Table 8.

TABLE 8
ABM Permutations in Tetravalent TBMs

| Tetravalent | | | | |
|---|---|---|---|---|
| Configuration | X | Y | Z | A |
| Tv 1 | ABM1/ABM4 | ABM1/ABM4 | ABM3/ABM6 | ABM2/ABM5 |
| Tv 2 | ABM1/ABM4 | ABM1/ABM4 | ABM2/ABM5 | ABM3/ABM6 |
| Tv 3 | ABM1/ABM4 | ABM3/ABM6 | ABM1/ABM4 | ABM2/ABM5 |
| Tv 4 | ABM1/ABM4 | ABM2/ABM5 | ABM1/ABM4 | ABM3/ABM6 |
| Tv 5 | ABM1/ABM4 | ABM3/ABM6 | ABM2/ABM5 | ABM1/ABM4 |
| Tv 6 | ABM1/ABM4 | ABM2/ABM5 | ABM3/ABM6 | ABM1/ABM4 |
| Tv 7 | ABM3/ABM6 | ABM1/ABM4 | ABM1/ABM4 | ABM2/ABM5 |
| Tv 8 | ABM2/ABM5 | ABM1/ABM4 | ABM1/ABM4 | ABM3/ABM6 |
| Tv 9 | ABM3/ABM6 | ABM1/ABM4 | ABM2/ABM5 | ABM1/ABM4 |
| Tv 10 | ABM2/ABM5 | ABM1/ABM4 | ABM3/ABM6 | ABM1/ABM4 |
| Tv 11 | ABM3/ABM6 | ABM2/ABM5 | ABM1/ABM4 | ABM1/ABM4 |
| Tv 12 | ABM2/ABM5 | ABM3/ABM6 | ABM1/ABM4 | ABM1/ABM4 |
| Tv 13 | ABM1/ABM4 | ABM3/ABM6 | ABM2/ABM5 | ABM2/ABM5 |
| Tv 14 | ABM1/ABM4 | ABM2/ABM5 | ABM3/ABM6 | ABM2/ABM5 |
| Tv 15 | ABM1/ABM4 | ABM2/ABM5 | ABM2/ABM5 | ABM3/ABM6 |
| Tv 16 | ABM3/ABM6 | ABM1/ABM4 | ABM2/ABM5 | ABM2/ABM5 |
| Tv 17 | ABM2/ABM5 | ABM1/ABM4 | ABM3/ABM6 | ABM2/ABM5 |
| Tv 18 | ABM2/ABM5 | ABM1/ABM4 | ABM2/ABM5 | ABM3/ABM6 |
| Tv 19 | ABM3/ABM6 | ABM2/ABM5 | ABM1/ABM4 | ABM2/ABM5 |
| Tv 20 | ABM2/ABM5 | ABM3/ABM6 | ABM1/ABM4 | ABM2/ABM5 |
| Tv 21 | ABM2/ABM5 | ABM2/ABM5 | ABM1/ABM4 | ABM3/ABM6 |
| Tv 22 | ABM3/ABM6 | ABM2/ABM5 | ABM2/ABM5 | ABM1/ABM4 |
| Tv 23 | ABM2/ABM5 | ABM3/ABM6 | ABM2/ABM5 | ABM1/ABM4 |
| Tv 24 | ABM2/ABM5 | ABM2/ABM5 | ABM3/ABM6 | ABM1/ABM4 |

### 7.5.3. Exemplary Pentavalent TBMs

The TBMs of the disclosure can be pentavalent, e.g., they can have five antigen-binding domains, one two, or three of which finds a first target antigen, one two, or three of which binds a second target antigen, and one two, or three of which binds a third target antigen.

An exemplary pentavalent TBM configuration is shown in FIG. 2T. In the TBMs of the disclosure, a non-immunoglobulin-based domain can substitute for a Fab and/or scFv in any of the configurations illustrated.

As depicted in FIG. 2T, a pentavalent TBM can comprise two half antibodies, one of which comprises two complete ABMs and the other of which comprises one complete ABM, the two halves paired through an Fc domain.

In the embodiment of FIG. 2T, the first (or left) half antibody comprises a Fab, an scFv, and an Fc region, and the second (or right) half antibody comprises a Fab, an Fc region, and an scFv. The first and second half antibodies are associated through the Fc regions forming an Fc domain.

In the configuration shown in FIG. 2T, for a first TBM having an ABM1, an ABM2, and an ABM3, each of X, Y, Z, A, and B represent an ABM1, an ABM2, or an ABM3, although not necessarily in that order, and provided that the TBM comprises at least one ABM1, one ABM2, and one ABM3. Likewise, for a second TBM having an ABM4, an ABM5, and an ABM6, each of X, Y, Z, A, and B represent an ABM4, an ABM5, or an ABM6, although not necessarily in that order, and provided that the TBM comprises at least one ABM4, one ABM5, and one ABM6.

Accordingly, the present disclosure provides pentavalent TBMs as shown in FIG. 2T, where X, Y, Z, A, and B are ABM1s, ABM2s, and ABM3s (for a first TBM) or ABM4s, ABM5s, and ABM6s (for a second TBM), as shown in Table 9.

TABLE 9
ABM Permutations in Pentavalent TBMs

| Pentavalent | | | | | |
|---|---|---|---|---|---|
| Configuration | X | Y | Z | A | B |
| Pv 1 | ABM1/ABM4 | ABM1/ABM4 | ABM1/ABM4 | ABM3/ABM6 | ABM2/ABM5 |
| Pv 2 | ABM1/ABM4 | ABM1/ABM4 | ABM1/ABM4 | ABM2/ABM5 | ABM3/ABM6 |
| Pv 3 | ABM1/ABM4 | ABM1/ABM4 | ABM3/ABM6 | ABM1/ABM4 | ABM2/ABM5 |
| Pv 4 | ABM1/ABM4 | ABM1/ABM4 | ABM2/ABM5 | ABM1/ABM4 | ABM3/ABM6 |
| Pv 5 | ABM1/ABM4 | ABM1/ABM4 | ABM3/ABM6 | ABM2/ABM5 | ABM1/ABM4 |
| Pv 6 | ABM1/ABM4 | ABM1/ABM4 | ABM2/ABM5 | ABM3/ABM6 | ABM1/ABM4 |
| Pv 7 | ABM1/ABM4 | ABM3/ABM6 | ABM1/ABM4 | ABM1/ABM4 | ABM2/ABM5 |
| Pv 8 | ABM1/ABM4 | ABM2/ABM5 | ABM1/ABM4 | ABM1/ABM4 | ABM3/ABM6 |
| Pv 9 | ABM1/ABM4 | ABM3/ABM6 | ABM1/ABM4 | ABM2/ABM5 | ABM1/ABM4 |
| Pv 10 | ABM1/ABM4 | ABM2/ABM5 | ABM1/ABM4 | ABM3/ABM6 | ABM1/ABM4 |
| Pv 11 | ABM1/ABM4 | ABM3/ABM6 | ABM2/ABM5 | ABM1/ABM4 | ABM1/ABM4 |
| Pv 12 | ABM1/ABM4 | ABM2/ABM5 | ABM3/ABM6 | ABM1/ABM4 | ABM1/ABM4 |
| Pv 13 | ABM3/ABM6 | ABM1/ABM4 | ABM1/ABM4 | ABM1/ABM4 | ABM2/ABM5 |
| Pv 14 | ABM2/ABM5 | ABM1/ABM4 | ABM1/ABM4 | ABM1/ABM4 | ABM3/ABM6 |
| Pv 15 | ABM3/ABM6 | ABM1/ABM4 | ABM1/ABM4 | ABM2/ABM5 | ABM1/ABM4 |
| Pv 16 | ABM2/ABM5 | ABM1/ABM4 | ABM1/ABM4 | ABM3/ABM6 | ABM1/ABM4 |
| Pv 17 | ABM3/ABM6 | ABM1/ABM4 | ABM2/ABM5 | ABM1/ABM4 | ABM1/ABM4 |
| Pv 18 | ABM2/ABM5 | ABM1/ABM4 | ABM3/ABM6 | ABM1/ABM4 | ABM1/ABM4 |
| Pv 19 | ABM3/ABM6 | ABM2/ABM5 | ABM1/ABM4 | ABM1/ABM4 | ABM1/ABM4 |
| Pv 20 | ABM2/ABM5 | ABM3/ABM6 | ABM1/ABM4 | ABM1/ABM4 | ABM1/ABM4 |
| Pv 21 | ABM1/ABM4 | ABM1/ABM4 | ABM3/ABM6 | ABM3/ABM6 | ABM2/ABM5 |
| Pv 22 | ABM1/ABM4 | ABM1/ABM4 | ABM3/ABM6 | ABM2/ABM5 | ABM3/ABM6 |
| Pv 23 | ABM1/ABM4 | ABM1/ABM4 | ABM2/ABM5 | ABM3/ABM6 | ABM3/ABM6 |
| Pv 24 | ABM1/ABM4 | ABM3/ABM6 | ABM1/ABM4 | ABM3/ABM6 | ABM2/ABM5 |
| Pv 25 | ABM1/ABM4 | ABM3/ABM6 | ABM1/ABM4 | ABM2/ABM5 | ABM3/ABM6 |
| Pv 26 | ABM1/ABM4 | ABM2/ABM5 | ABM1/ABM4 | ABM3/ABM6 | ABM3/ABM6 |
| Pv 27 | ABM1/ABM4 | ABM3/ABM6 | ABM3/ABM6 | ABM1/ABM4 | ABM2/ABM5 |
| Pv 28 | ABM1/ABM4 | ABM3/ABM6 | ABM2/ABM5 | ABM1/ABM4 | ABM3/ABM6 |
| Pv 29 | ABM1/ABM4 | ABM2/ABM5 | ABM3/ABM6 | ABM1/ABM4 | ABM3/ABM6 |
| Pv 30 | ABM1/ABM4 | ABM3/ABM6 | ABM3/ABM6 | ABM2/ABM5 | ABM1/ABM4 |
| Pv 31 | ABM1/ABM4 | ABM3/ABM6 | ABM2/ABM5 | ABM3/ABM6 | ABM1/ABM4 |
| Pv 32 | ABM1/ABM4 | ABM2/ABM5 | ABM3/ABM6 | ABM3/ABM6 | ABM1/ABM4 |
| Pv 33 | ABM3/ABM6 | ABM1/ABM4 | ABM1/ABM4 | ABM3/ABM6 | ABM2/ABM5 |
| Pv 34 | ABM3/ABM6 | ABM1/ABM4 | ABM1/ABM4 | ABM2/ABM5 | ABM3/ABM6 |
| Pv 35 | ABM2/ABM5 | ABM1/ABM4 | ABM1/ABM4 | ABM3/ABM6 | ABM3/ABM6 |
| Pv 36 | ABM3/ABM6 | ABM1/ABM4 | ABM3/ABM6 | ABM1/ABM4 | ABM2/ABM5 |
| Pv 37 | ABM3/ABM6 | ABM1/ABM4 | ABM2/ABM5 | ABM1/ABM4 | ABM3/ABM6 |
| Pv 38 | ABM2/ABM5 | ABM1/ABM4 | ABM3/ABM6 | ABM1/ABM4 | ABM3/ABM6 |
| Pv 39 | ABM3/ABM6 | ABM1/ABM4 | ABM3/ABM6 | ABM2/ABM5 | ABM1/ABM4 |
| Pv 40 | ABM3/ABM6 | ABM1/ABM4 | ABM2/ABM5 | ABM3/ABM6 | ABM1/ABM4 |
| Pv 41 | ABM2/ABM5 | ABM1/ABM4 | ABM3/ABM6 | ABM3/ABM6 | ABM1/ABM4 |
| Pv 42 | ABM3/ABM6 | ABM3/ABM6 | ABM1/ABM4 | ABM1/ABM4 | ABM2/ABM5 |
| Pv 43 | ABM3/ABM6 | ABM2/ABM5 | ABM1/ABM4 | ABM1/ABM4 | ABM3/ABM6 |
| Pv 44 | ABM2/ABM5 | ABM3/ABM6 | ABM1/ABM4 | ABM1/ABM4 | ABM3/ABM6 |
| Pv 45 | ABM3/ABM6 | ABM3/ABM6 | ABM1/ABM4 | ABM2/ABM5 | ABM1/ABM4 |
| Pv 46 | ABM3/ABM6 | ABM2/ABM5 | ABM1/ABM4 | ABM3/ABM6 | ABM1/ABM4 |
| Pv 47 | ABM2/ABM5 | ABM3/ABM6 | ABM1/ABM4 | ABM3/ABM6 | ABM1/ABM4 |
| Pv 48 | ABM3/ABM6 | ABM3/ABM6 | ABM2/ABM5 | ABM1/ABM4 | ABM1/ABM4 |
| Pv 49 | ABM3/ABM6 | ABM2/ABM5 | ABM3/ABM6 | ABM1/ABM4 | ABM1/ABM4 |
| Pv 50 | ABM2/ABM5 | ABM3/ABM6 | ABM3/ABM6 | ABM1/ABM4 | ABM1/ABM4 |
| Pv 51 | ABM1/ABM4 | ABM1/ABM4 | ABM3/ABM6 | ABM2/ABM5 | ABM2/ABM5 |
| Pv 52 | ABM1/ABM4 | ABM1/ABM4 | ABM2/ABM5 | ABM3/ABM6 | ABM2/ABM5 |
| Pv 53 | ABM1/ABM4 | ABM1/ABM4 | ABM2/ABM5 | ABM2/ABM5 | ABM3/ABM6 |
| Pv 54 | ABM1/ABM4 | ABM3/ABM6 | ABM1/ABM4 | ABM2/ABM5 | ABM2/ABM5 |
| Pv 55 | ABM1/ABM4 | ABM2/ABM5 | ABM1/ABM4 | ABM3/ABM6 | ABM2/ABM5 |
| Pv 56 | ABM1/ABM4 | ABM2/ABM5 | ABM1/ABM4 | ABM2/ABM5 | ABM3/ABM6 |
| Pv 57 | ABM1/ABM4 | ABM3/ABM6 | ABM2/ABM5 | ABM1/ABM4 | ABM2/ABM5 |
| Pv 58 | ABM1/ABM4 | ABM2/ABM5 | ABM3/ABM6 | ABM1/ABM4 | ABM2/ABM5 |
| Pv 59 | ABM1/ABM4 | ABM2/ABM5 | ABM2/ABM5 | ABM1/ABM4 | ABM3/ABM6 |
| Pv 60 | ABM1/ABM4 | ABM3/ABM6 | ABM2/ABM5 | ABM2/ABM5 | ABM1/ABM4 |
| Pv 61 | ABM1/ABM4 | ABM2/ABM5 | ABM3/ABM6 | ABM2/ABM5 | ABM1/ABM4 |
| Pv 62 | ABM1/ABM4 | ABM2/ABM5 | ABM2/ABM5 | ABM3/ABM6 | ABM1/ABM4 |
| Pv 63 | ABM3/ABM6 | ABM1/ABM4 | ABM1/ABM4 | ABM2/ABM5 | ABM2/ABM5 |
| Pv 64 | ABM2/ABM5 | ABM1/ABM4 | ABM1/ABM4 | ABM3/ABM6 | ABM2/ABM5 |
| Pv 65 | ABM2/ABM5 | ABM1/ABM4 | ABM1/ABM4 | ABM2/ABM5 | ABM3/ABM6 |
| Pv 66 | ABM3/ABM6 | ABM1/ABM4 | ABM2/ABM5 | ABM1/ABM4 | ABM2/ABM5 |
| Pv 67 | ABM2/ABM5 | ABM1/ABM4 | ABM3/ABM6 | ABM1/ABM4 | ABM2/ABM5 |
| Pv 68 | ABM2/ABM5 | ABM1/ABM4 | ABM2/ABM5 | ABM1/ABM4 | ABM3/ABM6 |
| Pv 69 | ABM3/ABM6 | ABM1/ABM4 | ABM2/ABM5 | ABM2/ABM5 | ABM1/ABM4 |
| Pv 70 | ABM2/ABM5 | ABM1/ABM4 | ABM3/ABM6 | ABM2/ABM5 | ABM1/ABM4 |
| Pv 71 | ABM2/ABM5 | ABM1/ABM4 | ABM2/ABM5 | ABM3/ABM6 | ABM1/ABM4 |
| Pv 72 | ABM3/ABM6 | ABM2/ABM5 | ABM1/ABM4 | ABM1/ABM4 | ABM2/ABM5 |
| Pv 73 | ABM2/ABM5 | ABM3/ABM6 | ABM1/ABM4 | ABM1/ABM4 | ABM2/ABM5 |
| Pv 74 | ABM2/ABM5 | ABM2/ABM5 | ABM1/ABM4 | ABM1/ABM4 | ABM3/ABM6 |
| Pv 75 | ABM3/ABM6 | ABM2/ABM5 | ABM1/ABM4 | ABM2/ABM5 | ABM1/ABM4 |
| Pv 76 | ABM2/ABM5 | ABM3/ABM6 | ABM1/ABM4 | ABM2/ABM5 | ABM1/ABM4 |
| Pv 77 | ABM2/ABM5 | ABM2/ABM5 | ABM1/ABM4 | ABM3/ABM6 | ABM1/ABM4 |
| Pv 78 | ABM3/ABM6 | ABM2/ABM5 | ABM2/ABM5 | ABM1/ABM4 | ABM1/ABM4 |
| Pv 79 | ABM2/ABM5 | ABM3/ABM6 | ABM2/ABM5 | ABM1/ABM4 | ABM1/ABM4 |
| Pv 80 | ABM2/ABM5 | ABM2/ABM5 | ABM3/ABM6 | ABM1/ABM4 | ABM1/ABM4 |
| Pv 81 | ABM1/ABM4 | ABM3/ABM6 | ABM2/ABM5 | ABM2/ABM5 | ABM2/ABM5 |
| Pv 82 | ABM1/ABM4 | ABM2/ABM5 | ABM3/ABM6 | ABM2/ABM5 | ABM2/ABM5 |
| Pv 83 | ABM1/ABM4 | ABM2/ABM5 | ABM2/ABM5 | ABM3/ABM6 | ABM2/ABM5 |
| Pv 84 | ABM1/ABM4 | ABM2/ABM5 | ABM2/ABM5 | ABM2/ABM5 | ABM3/ABM6 |
| Pv 85 | ABM3/ABM6 | ABM1/ABM4 | ABM2/ABM5 | ABM2/ABM5 | ABM2/ABM5 |
| Pv 86 | ABM2/ABM5 | ABM1/ABM4 | ABM3/ABM6 | ABM2/ABM5 | ABM2/ABM5 |
| Pv 87 | ABM2/ABM5 | ABM1/ABM4 | ABM2/ABM5 | ABM3/ABM6 | ABM2/ABM5 |
| Pv 88 | ABM2/ABM5 | ABM1/ABM4 | ABM2/ABM5 | ABM2/ABM5 | ABM3/ABM6 |
| Pv 89 | ABM3/ABM6 | ABM2/ABM5 | ABM1/ABM4 | ABM2/ABM5 | ABM2/ABM5 |
| Pv 90 | ABM2/ABM5 | ABM3/ABM6 | ABM1/ABM4 | ABM2/ABM5 | ABM2/ABM5 |
| Pv 91 | ABM2/ABM5 | ABM2/ABM5 | ABM1/ABM4 | ABM3/ABM6 | ABM2/ABM5 |
| Pv 92 | ABM2/ABM5 | ABM2/ABM5 | ABM1/ABM4 | ABM2/ABM5 | ABM3/ABM6 |
| Pv 93 | ABM3/ABM6 | ABM2/ABM5 | ABM2/ABM5 | ABM1/ABM4 | ABM2/ABM5 |
| Pv 94 | ABM2/ABM5 | ABM3/ABM6 | ABM2/ABM5 | ABM1/ABM4 | ABM2/ABM5 |
| Pv 95 | ABM2/ABM5 | ABM2/ABM5 | ABM3/ABM6 | ABM1/ABM4 | ABM2/ABM5 |
| Pv 96 | ABM2/ABM5 | ABM2/ABM5 | ABM2/ABM5 | ABM1/ABM4 | ABM3/ABM6 |
| Pv 97 | ABM3/ABM6 | ABM2/ABM5 | ABM2/ABM5 | ABM2/ABM5 | ABM1/ABM4 |
| Pv 98 | ABM2/ABM5 | ABM3/ABM6 | ABM2/ABM5 | ABM2/ABM5 | ABM1/ABM4 |
| Pv 99 | ABM2/ABM5 | ABM2/ABM5 | ABM3/ABM6 | ABM2/ABM5 | ABM1/ABM4 |
| Pv 100 | ABM2/ABM5 | ABM2/ABM5 | ABM2/ABM5 | ABM3/ABM6 | ABM1/ABM4 |

### 7.5.4. Exemplary Hexavalent TBMs

The TBMs of the disclosure can be hexavalent, e.g., they can have six antigen-binding modules, one, two, three, or four of which binds a first target antigen, one, two, three, or four of which binds a second target antigen, and one, two, three, or four of which binds a third target antigen.

Exemplary hexavalent TBM configurations are shown in FIGS. 2U-2V. In the TBMs of the disclosure, a non-immunoglobulin-based domain can substitute for a Fab and/or scFv in any of the configurations illustrated.

As depicted in FIGS. 2U-2V, a hexavalent TBM can comprise two half antibodies, one of which comprises two complete ABMs and the other of which comprises one complete ABM, the two halves paired through an Fc domain.

In the embodiment of FIG. 2U, the first (or left) half antibody comprises a Fab, a second Fab, an Fc region, and an scFv, and the second (or right) half antibody comprises a Fab, a second Fab, an Fc region, and an scFv. The first and second half antibodies are associated through the Fc regions forming an Fc domain.

In the embodiment of FIG. 2V, the first (or left) half antibody comprises a first Fv, a second Fv, a third Fv, and an Fc region, and the second (or right) half antibody comprises a first Fv, a second Fv, a third Fv, and an Fc region. The first and second half antibodies are associated through the Fc regions forming an Fc domain.

In the configuration shown in FIGS. 2U-2V, for a first TBM having an ABM1, an ABM2, and an ABM3, each of X, Y, Z, A, B, and C represent an ABM1, an ABM2, or an ABM3, although not necessarily in that order, and provided that the TBM comprises at least one ABM1, one ABM2, and one ABM3. Likewise, for a second TBM having an ABM4, an ABM5, and an ABM6, each of X, Y, Z, A, B, and C represent an ABM4, an ABM5, or an ABM6, although not necessarily in that order, and provided that the TBM comprises at least one ABM4, one ABM5, and one ABM6.

Accordingly, in the present disclosure provides hexavalent TBMs as shown in any one of FIGS. 2U-2V, where X, Y, Z, A, B, and C are ABM1s, ABM2s, and ABM3s (for a first TBM) or ABM4s, ABM5s, and ABM6s (for a second TBM), as shown in Table 10.

TABLE 10
ABM Permutations in Hexavalent TBMs

| Hexavalent | | | | | | |
|---|---|---|---|---|---|---|
| Configuration | X | Y | Z | A | B | C |
| Hv 1 | ABM1/ | ABM1/ | ABM1/ | ABM1/ | ABM3/ | ABM2/ |
| | ABM4 | ABM4 | ABM4 | ABM4 | ABM6 | ABM5 |
| Hv 2 | ABM1/ | ABM1/ | ABM1/ | ABM1/ | ABM2/ | ABM3/ |
| | ABM4 | ABM4 | ABM4 | ABM4 | ABM5 | ABM6 |
| Hv 3 | ABM1/ | ABM1/ | ABM1/ | ABM3/ | ABM1/ | ABM2/ |
| | ABM4 | ABM4 | ABM4 | ABM6 | ABM4 | ABM5 |
| Hv 4 | ABM1/ | ABM1/ | ABM1/ | ABM2/ | ABM1/ | ABM3/ |
| | ABM4 | ABM4 | ABM4 | ABM5 | ABM4 | ABM6 |
| Hv 5 | ABM1/ | ABM1/ | ABM1/ | ABM3/ | ABM2/ | ABM1/ |
| | ABM4 | ABM4 | ABM4 | ABM6 | ABM5 | ABM4 |
| Hv 6 | ABM1/ | ABM1/ | ABM1/ | ABM2/ | ABM3/ | ABM1/ |
| | ABM4 | ABM4 | ABM4 | ABM5 | ABM6 | ABM4 |
| Hv 7 | ABM1/ | ABM1/ | ABM3/ | ABM1/ | ABM1/ | ABM2/ |
| | ABM4 | ABM4 | ABM6 | ABM4 | ABM4 | ABM5 |
| Hv 8 | ABM1/ | ABM1/ | ABM2/ | ABM1/ | ABM1/ | ABM3/ |
| | ABM4 | ABM4 | ABM5 | ABM4 | ABM4 | ABM6 |
| Hv 9 | ABM1/ | ABM1/ | ABM3/ | ABM1/ | ABM2/ | ABM1/ |
| | ABM4 | ABM4 | ABM6 | ABM4 | ABM5 | ABM4 |
| Hv 10 | ABM1/ | ABM1/ | ABM2/ | ABM1/ | ABM3/ | ABM1/ |
| | ABM4 | ABM4 | ABM5 | ABM4 | ABM6 | ABM4 |
| Hv 11 | ABM1/ | ABM1/ | ABM3/ | ABM2/ | ABM1/ | ABM1/ |
| | ABM4 | ABM4 | ABM6 | ABM5 | ABM4 | ABM4 |
| Hv 12 | ABM1/ | ABM1/ | ABM2/ | ABM3/ | ABM1/ | ABM1/ |
| | ABM4 | ABM4 | ABM5 | ABM6 | ABM4 | ABM4 |
| Hv 13 | ABM1/ | ABM3/ | ABM1/ | ABM1/ | ABM1/ | ABM2/ |
| | ABM4 | ABM6 | ABM4 | ABM4 | ABM4 | ABM5 |
| Hv 14 | ABM1/ | ABM2/ | ABM1/ | ABM1/ | ABM1/ | ABM3/ |
| | ABM4 | ABM5 | ABM4 | ABM4 | ABM4 | ABM6 |
| Hv 15 | ABM1/ | ABM3/ | ABM1/ | ABM1/ | ABM2/ | ABM1/ |
| | ABM4 | ABM6 | ABM4 | ABM4 | ABM5 | ABM4 |
| Hv 16 | ABM1/ | ABM2/ | ABM1/ | ABM1/ | ABM3/ | ABM1/ |
| | ABM4 | ABM5 | ABM4 | ABM4 | ABM6 | ABM4 |
| Hv 17 | ABM1/ | ABM3/ | ABM1/ | ABM2/ | ABM1/ | ABM1/ |
| | ABM4 | ABM6 | ABM4 | ABM5 | ABM4 | ABM4 |
| Hv 18 | ABM1/ | ABM2/ | ABM1/ | ABM3/ | ABM1/ | ABM1/ |
| | ABM4 | ABM5 | ABM4 | ABM6 | ABM4 | ABM4 |
| Hv 19 | ABM1/ | ABM3/ | ABM2/ | ABM1/ | ABM1/ | ABM1/ |
| | ABM4 | ABM6 | ABM5 | ABM4 | ABM4 | ABM4 |
| Hv 20 | ABM1/ | ABM2/ | ABM3/ | ABM1/ | ABM1/ | ABM1/ |
| | ABM4 | ABM5 | ABM6 | ABM4 | ABM4 | ABM4 |
| Hv 21 | ABM3/ | ABM1/ | ABM1/ | ABM1/ | ABM1/ | ABM2/ |
| | ABM6 | ABM4 | ABM4 | ABM4 | ABM4 | ABM5 |
| Hv 22 | ABM2/ | ABM1/ | ABM1/ | ABM1/ | ABM1/ | ABM3/ |
| | ABM5 | ABM4 | ABM4 | ABM4 | ABM4 | ABM6 |
| Hv 23 | ABM3/ | ABM1/ | ABM1/ | ABM1/ | ABM2/ | ABM1/ |
| | ABM6 | ABM4 | ABM4 | ABM4 | ABM5 | ABM4 |
| Hv 24 | ABM2/ | ABM1/ | ABM1/ | ABM1/ | ABM3/ | ABM1/ |
| | ABM5 | ABM4 | ABM4 | ABM4 | ABM6 | ABM4 |
| Hv 25 | ABM3/ | ABM1/ | ABM1/ | ABM2/ | ABM1/ | ABM1/ |
| | ABM6 | ABM4 | ABM4 | ABM5 | ABM4 | ABM4 |
| Hv 26 | ABM2/ | ABM1/ | ABM1/ | ABM3/ | ABM1/ | ABM1/ |
| | ABM5 | ABM4 | ABM4 | ABM6 | ABM4 | ABM4 |
| Hv 27 | ABM3/ | ABM1/ | ABM2/ | ABM1/ | ABM1/ | ABM1/ |
| | ABM6 | ABM4 | ABM5 | ABM4 | ABM4 | ABM4 |
| Hv 28 | ABM2/ | ABM1/ | ABM3/ | ABM1/ | ABM1/ | ABM1/ |
| | ABM5 | ABM4 | ABM6 | ABM4 | ABM4 | ABM4 |
| Hv 29 | ABM3/ | ABM2/ | ABM1/ | ABM1/ | ABM1/ | ABM1/ |
| | ABM6 | ABM5 | ABM4 | ABM4 | ABM4 | ABM4 |
| Hv 30 | ABM2/ | ABM3/ | ABM1/ | ABM1/ | ABM1/ | ABM1/ |
| | ABM5 | ABM6 | ABM4 | ABM4 | ABM4 | ABM4 |
| Hv 31 | ABM1/ | ABM1/ | ABM1/ | ABM3/ | ABM3/ | ABM2/ |
| | ABM4 | ABM4 | ABM4 | ABM6 | ABM6 | ABM5 |
| Hv 32 | ABM1/ | ABM1/ | ABM1/ | ABM3/ | ABM2/ | ABM3/ |
| | ABM4 | ABM4 | ABM4 | ABM6 | ABM5 | ABM6 |
| Hv 33 | ABM1/ | ABM1/ | ABM1/ | ABM2/ | ABM3/ | ABM3/ |
| | ABM4 | ABM4 | ABM4 | ABM5 | ABM6 | ABM6 |
| Hv 34 | ABM1/ | ABM1/ | ABM3/ | ABM1/ | ABM3/ | ABM2/ |
| | ABM4 | ABM4 | ABM6 | ABM4 | ABM6 | ABM5 |
| Hv 35 | ABM1/ | ABM1/ | ABM3/ | ABM1/ | ABM2/ | ABM3/ |
| | ABM4 | ABM4 | ABM6 | ABM4 | ABM5 | ABM6 |
| Hv 36 | ABM1/ | ABM1/ | ABM2/ | ABM1/ | ABM3/ | ABM3/ |
| | ABM4 | ABM4 | ABM5 | ABM4 | ABM6 | ABM6 |
| Hv 37 | ABM1/ | ABM1/ | ABM3/ | ABM3/ | ABM1/ | ABM2/ |
| | ABM4 | ABM4 | ABM6 | ABM6 | ABM4 | ABM5 |
| Hv 38 | ABM1/ | ABM1/ | ABM3/ | ABM2/ | ABM1/ | ABM3/ |
| | ABM4 | ABM4 | ABM6 | ABM5 | ABM4 | ABM6 |
| Hv 39 | ABM1/ | ABM1/ | ABM2/ | ABM3/ | ABM1/ | ABM3/ |
| | ABM4 | ABM4 | ABM5 | ABM6 | ABM4 | ABM6 |
| Hv 40 | ABM1/ | ABM1/ | ABM3/ | ABM3/ | ABM2/ | ABM1/ |
| | ABM4 | ABM4 | ABM6 | ABM6 | ABM5 | ABM4 |
| Hv 41 | ABM1/ | ABM1/ | ABM3/ | ABM2/ | ABM3/ | ABM1/ |
| | ABM4 | ABM4 | ABM6 | ABM5 | ABM6 | ABM4 |
| Hv 42 | ABM1/ | ABM1/ | ABM2/ | ABM3/ | ABM3/ | ABM1/ |
| | ABM4 | ABM4 | ABM5 | ABM6 | ABM6 | ABM4 |
| Hv 43 | ABM1/ | ABM3/ | ABM1/ | ABM1/ | ABM3/ | ABM2/ |
| | ABM4 | ABM6 | ABM4 | ABM4 | ABM6 | ABM5 |
| Hv 44 | ABM1/ | ABM3/ | ABM1/ | ABM1/ | ABM2/ | ABM3/ |
| | ABM4 | ABM6 | ABM4 | ABM4 | ABM5 | ABM6 |
| Hv 45 | ABM1/ | ABM2/ | ABM1/ | ABM1/ | ABM3/ | ABM3/ |
| | ABM4 | ABM5 | ABM4 | ABM4 | ABM6 | ABM6 |
| Hv 46 | ABM1/ | ABM3/ | ABM1/ | ABM3/ | ABM1/ | ABM2/ |
| | ABM4 | ABM6 | ABM4 | ABM6 | ABM4 | ABM5 |
| Hv 47 | ABM1/ | ABM3/ | ABM1/ | ABM2/ | ABM1/ | ABM3/ |
| | ABM4 | ABM6 | ABM4 | ABM5 | ABM4 | ABM6 |
| Hv 48 | ABM1/ | ABM2/ | ABM1/ | ABM3/ | ABM1/ | ABM3/ |
| | ABM4 | ABM5 | ABM4 | ABM6 | ABM4 | ABM6 |
| Hv 49 | ABM1/ | ABM3/ | ABM1/ | ABM3/ | ABM2/ | ABM1/ |
| | ABM4 | ABM6 | ABM4 | ABM6 | ABM5 | ABM4 |
| Hv 50 | ABM1/ | ABM3/ | ABM1/ | ABM2/ | ABM3/ | ABM1/ |
| | ABM4 | ABM6 | ABM4 | ABM5 | ABM6 | ABM4 |
| Hv 51 | ABM1/ | ABM2/ | ABM1/ | ABM3/ | ABM3/ | ABM1/ |
| | ABM4 | ABM5 | ABM4 | ABM6 | ABM6 | ABM4 |
| Hv 52 | ABM1/ | ABM3/ | ABM3/ | ABM1/ | ABM1/ | ABM2/ |
| | ABM4 | ABM6 | ABM6 | ABM4 | ABM4 | ABM5 |
| Hv 53 | ABM1/ | ABM3/ | ABM2/ | ABM1/ | ABM1/ | ABM3/ |
| | ABM4 | ABM6 | ABM5 | ABM4 | ABM4 | ABM6 |
| Hv 54 | ABM1/ | ABM2/ | ABM3/ | ABM1/ | ABM1/ | ABM3/ |
| | ABM4 | ABM5 | ABM6 | ABM4 | ABM4 | ABM6 |
| Hv 55 | ABM1/ | ABM3/ | ABM3/ | ABM1/ | ABM2/ | ABM1/ |
| | ABM4 | ABM6 | ABM6 | ABM4 | ABM5 | ABM4 |
| Hv 56 | ABM1/ | ABM3/ | ABM2/ | ABM1/ | ABM3/ | ABM1/ |
| | ABM4 | ABM6 | ABM5 | ABM4 | ABM6 | ABM4 |
| Hv 57 | ABM1/ | ABM2/ | ABM3/ | ABM1/ | ABM3/ | ABM1/ |
| | ABM4 | ABM5 | ABM6 | ABM4 | ABM6 | ABM4 |
| Hv 58 | ABM1/ | ABM3/ | ABM3/ | ABM2/ | ABM1/ | ABM1/ |
| | ABM4 | ABM6 | ABM6 | ABM5 | ABM4 | ABM4 |
| Hv 59 | ABM1/ | ABM3/ | ABM2/ | ABM3/ | ABM1/ | ABM1/ |
| | ABM4 | ABM6 | ABM5 | ABM6 | ABM4 | ABM4 |
| Hv 60 | ABM1/ | ABM2/ | ABM3/ | ABM3/ | ABM1/ | ABM1/ |
| | ABM4 | ABM5 | ABM6 | ABM6 | ABM4 | ABM4 |
| Hv 61 | ABM3/ | ABM1/ | ABM1/ | ABM1/ | ABM3/ | ABM2/ |
| | ABM6 | ABM4 | ABM4 | ABM4 | ABM6 | ABM5 |
| Hv 62 | ABM3/ | ABM1/ | ABM1/ | ABM1/ | ABM2/ | ABM3/ |
| | ABM6 | ABM4 | ABM4 | ABM4 | ABM5 | ABM6 |
| Hv 63 | ABM2/ | ABM1/ | ABM1/ | ABM1/ | ABM3/ | ABM3/ |
| | ABM5 | ABM4 | ABM4 | ABM4 | ABM6 | ABM6 |
| Hv 64 | ABM3/ | ABM1/ | ABM1/ | ABM3/ | ABM1/ | ABM2/ |
| | ABM6 | ABM4 | ABM4 | ABM6 | ABM4 | ABM5 |
| Hv 65 | ABM3/ | ABM1/ | ABM1/ | ABM2/ | ABM1/ | ABM3/ |
| | ABM6 | ABM4 | ABM4 | ABM5 | ABM4 | ABM6 |
| Hv 66 | ABM2/ | ABM1/ | ABM1/ | ABM3/ | ABM1/ | ABM3/ |
| | ABM5 | ABM4 | ABM4 | ABM6 | ABM4 | ABM6 |
| Hv 67 | ABM3/ | ABM1/ | ABM1/ | ABM3/ | ABM2/ | ABM1/ |
| | ABM6 | ABM4 | ABM4 | ABM6 | ABM5 | ABM4 |
| Hv 68 | ABM3/ | ABM1/ | ABM1/ | ABM2/ | ABM3/ | ABM1/ |
| | ABM6 | ABM4 | ABM4 | ABM5 | ABM6 | ABM4 |
| Hv 69 | ABM2/ | ABM1/ | ABM1/ | ABM3/ | ABM3/ | ABM1/ |
| | ABM5 | ABM4 | ABM4 | ABM6 | ABM6 | ABM4 |
| Hv 70 | ABM3/ | ABM1/ | ABM3/ | ABM1/ | ABM1/ | ABM2/ |
| | ABM6 | ABM4 | ABM6 | ABM4 | ABM4 | ABM5 |
| Hv 71 | ABM3/ | ABM1/ | ABM2/ | ABM1/ | ABM1/ | ABM3/ |
| | ABM6 | ABM4 | ABM5 | ABM4 | ABM4 | ABM6 |
| Hv 72 | ABM2/ | ABM1/ | ABM3/ | ABM1/ | ABM1/ | ABM3/ |
| | ABM5 | ABM4 | ABM6 | ABM4 | ABM4 | ABM6 |
| Hv 73 | ABM3/ | ABM1/ | ABM3/ | ABM1/ | ABM2/ | ABM1/ |
| | ABM6 | ABM4 | ABM6 | ABM4 | ABM5 | ABM4 |
| Hv 74 | ABM3/ | ABM1/ | ABM2/ | ABM1/ | ABM3/ | ABM1/ |
| | ABM6 | ABM4 | ABM5 | ABM4 | ABM6 | ABM4 |
| Hv 75 | ABM2/ | ABM1/ | ABM3/ | ABM1/ | ABM3/ | ABM1/ |
| | ABM5 | ABM4 | ABM6 | ABM4 | ABM6 | ABM4 |
| Hv 76 | ABM3/ | ABM1/ | ABM3/ | ABM2/ | ABM1/ | ABM1/ |
| | ABM6 | ABM4 | ABM6 | ABM5 | ABM4 | ABM4 |
| Hv 77 | ABM3/ | ABM1/ | ABM2/ | ABM3/ | ABM1/ | ABM1/ |
| | ABM6 | ABM4 | ABM5 | ABM6 | ABM4 | ABM4 |
| Hv 78 | ABM2/ | ABM1/ | ABM3/ | ABM3/ | ABM1/ | ABM1/ |
| | ABM5 | ABM4 | ABM6 | ABM6 | ABM4 | ABM4 |
| Hv 79 | ABM3/ | ABM3/ | ABM1/ | ABM1/ | ABM1/ | ABM2/ |
| | ABM6 | ABM6 | ABM4 | ABM4 | ABM4 | ABM5 |
| Hv 80 | ABM3/ | ABM2/ | ABM1/ | ABM1/ | ABM1/ | ABM3/ |
| | ABM6 | ABM5 | ABM4 | ABM4 | ABM4 | ABM6 |
| Hv 81 | ABM2/ | ABM3/ | ABM1/ | ABM1/ | ABM1/ | ABM3/ |
| | ABM5 | ABM6 | ABM4 | ABM4 | ABM4 | ABM6 |
| Hv 82 | ABM3/ | ABM3/ | ABM1/ | ABM1/ | ABM2/ | ABM1/ |
| | ABM6 | ABM6 | ABM4 | ABM4 | ABM5 | ABM4 |
| Hv 83 | ABM3/ | ABM2/ | ABM1/ | ABM1/ | ABM3/ | ABM1/ |
| | ABM6 | ABM5 | ABM4 | ABM4 | ABM6 | ABM4 |
| Hv 84 | ABM2/ | ABM3/ | ABM1/ | ABM1/ | ABM3/ | ABM1/ |
| | ABM5 | ABM6 | ABM4 | ABM4 | ABM6 | ABM4 |
| Hv 85 | ABM3/ | ABM3/ | ABM1/ | ABM2/ | ABM1/ | ABM1/ |
| | ABM6 | ABM6 | ABM4 | ABM5 | ABM4 | ABM4 |
| Hv 86 | ABM3/ | ABM2/ | ABM1/ | ABM3/ | ABM1/ | ABM1/ |
| | ABM6 | ABM5 | ABM4 | ABM6 | ABM4 | ABM4 |
| Hv 87 | ABM2/ | ABM3/ | ABM1/ | ABM3/ | ABM1/ | ABM1/ |
| | ABM5 | ABM6 | ABM4 | ABM6 | ABM4 | ABM4 |
| Hv 88 | ABM3/ | ABM3/ | ABM2/ | ABM1/ | ABM1/ | ABM1/ |
| | ABM6 | ABM6 | ABM5 | ABM4 | ABM4 | ABM4 |
| Hv 89 | ABM3/ | ABM2/ | ABM3/ | ABM1/ | ABM1/ | ABM1/ |
| | ABM6 | ABM5 | ABM6 | ABM4 | ABM4 | ABM4 |
| Hv 90 | ABM2/ | ABM3/ | ABM3/ | ABM1/ | ABM1/ | ABM1/ |
| | ABM5 | ABM6 | ABM6 | ABM4 | ABM4 | ABM4 |
| Hv 91 | ABM1/ | ABM1/ | ABM1/ | ABM3/ | ABM2/ | ABM2/ |
| | ABM4 | ABM4 | ABM4 | ABM6 | ABM5 | ABM5 |
| Hv 92 | ABM1/ | ABM1/ | ABM1/ | ABM2/ | ABM3/ | ABM2/ |
| | ABM4 | ABM4 | ABM4 | ABM5 | ABM6 | ABM5 |
| Hv 93 | ABM1/ | ABM1/ | ABM1/ | ABM2/ | ABM2/ | ABM3/ |
| | ABM4 | ABM4 | ABM4 | ABM5 | ABM5 | ABM6 |
| Hv 94 | ABM1/ | ABM1/ | ABM3/ | ABM1/ | ABM2/ | ABM2/ |
| | ABM4 | ABM4 | ABM6 | ABM4 | ABM5 | ABM5 |
| Hv 95 | ABM1/ | ABM1/ | ABM2/ | ABM1/ | ABM3/ | ABM2/ |
| | ABM4 | ABM4 | ABM5 | ABM4 | ABM6 | ABM5 |
| Hv 96 | ABM1/ | ABM1/ | ABM2/ | ABM1/ | ABM2/ | ABM3/ |
| | ABM4 | ABM4 | ABM5 | ABM4 | ABM5 | ABM6 |
| Hv 97 | ABM1/ | ABM1/ | ABM3/ | ABM2/ | ABM1/ | ABM2/ |
| | ABM4 | ABM4 | ABM6 | ABM5 | ABM4 | ABM5 |
| Hv 98 | ABM1/ | ABM1/ | ABM2/ | ABM3/ | ABM1/ | ABM2/ |
| | ABM4 | ABM4 | ABM5 | ABM6 | ABM4 | ABM5 |
| Hv 99 | ABM1/ | ABM1/ | ABM2/ | ABM2/ | ABM1/ | ABM3/ |
| | ABM4 | ABM4 | ABM5 | ABM5 | ABM4 | ABM6 |
| Hv 100 | ABM1/ | ABM1/ | ABM3/ | ABM2/ | ABM2/ | ABM1/ |
| | ABM4 | ABM4 | ABM6 | ABM5 | ABM5 | ABM4 |
| Hv 101 | ABM1/ | ABM1/ | ABM2/ | ABM3/ | ABM2/ | ABM1/ |
| | ABM4 | ABM4 | ABM5 | ABM6 | ABM5 | ABM4 |
| Hv 102 | ABM1/ | ABM1/ | ABM2/ | ABM2/ | ABM3/ | ABM1/ |
| | ABM4 | ABM4 | ABM5 | ABM5 | ABM6 | ABM4 |
| Hv 103 | ABM1/ | ABM3/ | ABM1/ | ABM1/ | ABM2/ | ABM2/ |
| | ABM4 | ABM6 | ABM4 | ABM4 | ABM5 | ABM5 |
| Hv 104 | ABM1/ | ABM2/ | ABM1/ | ABM1/ | ABM3/ | ABM2/ |
| | ABM4 | ABM5 | ABM4 | ABM4 | ABM6 | ABM5 |
| Hv 105 | ABM1/ | ABM2/ | ABM1/ | ABM1/ | ABM2/ | ABM3/ |
| | ABM4 | ABM5 | ABM4 | ABM4 | ABM5 | ABM6 |
| Hv 106 | ABM1/ | ABM3/ | ABM1/ | ABM2/ | ABM1/ | ABM2/ |
| | ABM4 | ABM6 | ABM4 | ABM5 | ABM4 | ABM5 |
| Hv 107 | ABM1/ | ABM2/ | ABM1/ | ABM3/ | ABM1/ | ABM2/ |
| | ABM4 | ABM5 | ABM4 | ABM6 | ABM4 | ABM5 |
| Hv 108 | ABM1/ | ABM2/ | ABM1/ | ABM2/ | ABM1/ | ABM3/ |
| | ABM4 | ABM5 | ABM4 | ABM5 | ABM4 | ABM6 |
| Hv 109 | ABM1/ | ABM3/ | ABM1/ | ABM2/ | ABM2/ | ABM1/ |
| | ABM4 | ABM6 | ABM4 | ABM5 | ABM5 | ABM4 |
| Hv 110 | ABM1/ | ABM2/ | ABM1/ | ABM3/ | ABM2/ | ABM1/ |
| | ABM4 | ABM5 | ABM4 | ABM6 | ABM5 | ABM4 |
| Hv 111 | ABM1/ | ABM2/ | ABM1/ | ABM2/ | ABM3/ | ABM1/ |
| | ABM4 | ABM5 | ABM4 | ABM5 | ABM6 | ABM4 |
| Hv 112 | ABM1/ | ABM3/ | ABM2/ | ABM1/ | ABM1/ | ABM2/ |
| | ABM4 | ABM6 | ABM5 | ABM4 | ABM4 | ABM5 |
| Hv 113 | ABM1/ | ABM2/ | ABM3/ | ABM1/ | ABM1/ | ABM2/ |
| | ABM4 | ABM5 | ABM6 | ABM4 | ABM4 | ABM5 |
| Hv 114 | ABM1/ | ABM2/ | ABM2/ | ABM1/ | ABM1/ | ABM3/ |
| | ABM4 | ABM5 | ABM5 | ABM4 | ABM4 | ABM6 |
| Hv 115 | ABM1/ | ABM3/ | ABM2/ | ABM1/ | ABM2/ | ABM1/ |
| | ABM4 | ABM6 | ABM5 | ABM4 | ABM5 | ABM4 |
| Hv 116 | ABM1/ | ABM2/ | ABM3/ | ABM1/ | ABM2/ | ABM1/ |
| | ABM4 | ABM5 | ABM6 | ABM4 | ABM5 | ABM4 |
| Hv 117 | ABM1/ | ABM2/ | ABM2/ | ABM1/ | ABM3/ | ABM1/ |
| | ABM4 | ABM5 | ABM5 | ABM4 | ABM6 | ABM4 |
| Hv 118 | ABM1/ | ABM3/ | ABM2/ | ABM2/ | ABM1/ | ABM1/ |
| | ABM4 | ABM6 | ABM5 | ABM5 | ABM4 | ABM4 |
| Hv 119 | ABM1/ | ABM2/ | ABM3/ | ABM2/ | ABM1/ | ABM1/ |
| | ABM4 | ABM5 | ABM6 | ABM5 | ABM4 | ABM4 |
| Hv 120 | ABM1/ | ABM2/ | ABM2/ | ABM3/ | ABM1/ | ABM1/ |
| | ABM4 | ABM5 | ABM5 | ABM6 | ABM4 | ABM4 |
| Hv 121 | ABM3/ | ABM1/ | ABM1/ | ABM1/ | ABM2/ | ABM2/ |
| | ABM6 | ABM4 | ABM4 | ABM4 | ABM5 | ABM5 |
| Hv 122 | ABM2/ | ABM1/ | ABM1/ | ABM1/ | ABM3/ | ABM2/ |
| | ABM5 | ABM4 | ABM4 | ABM4 | ABM6 | ABM5 |
| Hv 123 | ABM2/ | ABM1/ | ABM1/ | ABM1/ | ABM2/ | ABM3/ |
| | ABM5 | ABM4 | ABM4 | ABM4 | ABM5 | ABM6 |
| Hv 124 | ABM3/ | ABM1/ | ABM1/ | ABM2/ | ABM1/ | ABM2/ |
| | ABM6 | ABM4 | ABM4 | ABM5 | ABM4 | ABM5 |
| Hv 125 | ABM2/ | ABM1/ | ABM1/ | ABM3/ | ABM1/ | ABM2/ |
| | ABM5 | ABM4 | ABM4 | ABM6 | ABM4 | ABM5 |
| Hv 126 | ABM2/ | ABM1/ | ABM1/ | ABM2/ | ABM1/ | ABM3/ |
| | ABM5 | ABM4 | ABM4 | ABM5 | ABM4 | ABM6 |
| Hv 127 | ABM3/ | ABM1/ | ABM1/ | ABM2/ | ABM2/ | ABM1/ |
| | ABM6 | ABM4 | ABM4 | ABM5 | ABM5 | ABM4 |
| Hv 128 | ABM2/ | ABM1/ | ABM1/ | ABM3/ | ABM2/ | ABM1/ |
| | ABM5 | ABM4 | ABM4 | ABM6 | ABM5 | ABM4 |
| Hv 129 | ABM2/ | ABM1/ | ABM1/ | ABM2/ | ABM3/ | ABM1/ |
| | ABM5 | ABM4 | ABM4 | ABM5 | ABM6 | ABM4 |
| Hv 130 | ABM3/ | ABM1/ | ABM2/ | ABM1/ | ABM1/ | ABM2/ |
| | ABM6 | ABM4 | ABM5 | ABM4 | ABM4 | ABM5 |
| Hv 131 | ABM2/ | ABM1/ | ABM3/ | ABM1/ | ABM1/ | ABM2/ |
| | ABM5 | ABM4 | ABM6 | ABM4 | ABM4 | ABM5 |
| Hv 132 | ABM2/ | ABM1/ | ABM2/ | ABM1/ | ABM1/ | ABM3/ |
| | ABM5 | ABM4 | ABM5 | ABM4 | ABM4 | ABM6 |
| Hv 133 | ABM3/ | ABM1/ | ABM2/ | ABM1/ | ABM2/ | ABM1/ |
| | ABM6 | ABM4 | ABM5 | ABM4 | ABM5 | ABM4 |
| Hv 134 | ABM2/ | ABM1/ | ABM3/ | ABM1/ | ABM2/ | ABM1/ |
| | ABM5 | ABM4 | ABM6 | ABM4 | ABM5 | ABM4 |
| Hv 135 | ABM2/ | ABM1/ | ABM2/ | ABM1/ | ABM3/ | ABM1/ |
| | ABM5 | ABM4 | ABM5 | ABM4 | ABM6 | ABM4 |
| Hv 136 | ABM3/ | ABM1/ | ABM2/ | ABM2/ | ABM1/ | ABM1/ |
| | ABM6 | ABM4 | ABM5 | ABM5 | ABM4 | ABM4 |
| Hv 137 | ABM2/ | ABM1/ | ABM3/ | ABM2/ | ABM1/ | ABM1/ |
| | ABM5 | ABM4 | ABM6 | ABM5 | ABM4 | ABM4 |
| Hv 138 | ABM2/ | ABM1/ | ABM2/ | ABM3/ | ABM1/ | ABM1/ |
| | ABM5 | ABM4 | ABM5 | ABM6 | ABM4 | ABM4 |
| Hv 139 | ABM3/ | ABM2/ | ABM1/ | ABM1/ | ABM1/ | ABM2/ |
| | ABM6 | ABM5 | ABM4 | ABM4 | ABM4 | ABM5 |
| Hv 140 | ABM2/ | ABM3/ | ABM1/ | ABM1/ | ABM1/ | ABM2/ |
| | ABM5 | ABM6 | ABM4 | ABM4 | ABM4 | ABM5 |
| Hv 141 | ABM2/ | ABM2/ | ABM1/ | ABM1/ | ABM1/ | ABM3/ |
| | ABM5 | ABM5 | ABM4 | ABM4 | ABM4 | ABM6 |
| Hv 142 | ABM3/ | ABM2/ | ABM1/ | ABM1/ | ABM2/ | ABM1/ |
| | ABM6 | ABM5 | ABM4 | ABM4 | ABM5 | ABM4 |
| Hv 143 | ABM2/ | ABM3/ | ABM1/ | ABM1/ | ABM2/ | ABM1/ |
| | ABM5 | ABM6 | ABM4 | ABM4 | ABM5 | ABM4 |
| Hv 144 | ABM2/ | ABM2/ | ABM1/ | ABM1/ | ABM3/ | ABM1/ |
| | ABM5 | ABM5 | ABM4 | ABM4 | ABM6 | ABM4 |
| Hv 145 | ABM3/ | ABM2/ | ABM1/ | ABM2/ | ABM1/ | ABM1/ |
| | ABM6 | ABM5 | ABM4 | ABM5 | ABM4 | ABM4 |
| Hv 146 | ABM2/ | ABM3/ | ABM1/ | ABM2/ | ABM1/ | ABM1/ |
| | ABM5 | ABM6 | ABM4 | ABM5 | ABM4 | ABM4 |
| Hv 147 | ABM2/ | ABM2/ | ABM1/ | ABM3/ | ABM1/ | ABM1/ |
| | ABM5 | ABM5 | ABM4 | ABM6 | ABM4 | ABM4 |
| Hv 148 | ABM3/ | ABM2/ | ABM2/ | ABM1/ | ABM1/ | ABM1/ |
| | ABM6 | ABM5 | ABM5 | ABM4 | ABM4 | ABM4 |
| Hv 149 | ABM2/ | ABM3/ | ABM2/ | ABM1/ | ABM1/ | ABM1/ |
| | ABM5 | ABM6 | ABM5 | ABM4 | ABM4 | ABM4 |
| Hv 150 | ABM2/ | ABM2/ | ABM3/ | ABM1/ | ABM1/ | ABM1/ |
| | ABM5 | ABM5 | ABM6 | ABM4 | ABM4 | ABM4 |
| Hv 151 | ABM1/ | ABM1/ | ABM3/ | ABM3/ | ABM2/ | ABM2/ |
| | ABM4 | ABM4 | ABM6 | ABM6 | ABM5 | ABM5 |
| Hv 152 | ABM1/ | ABM1/ | ABM3/ | ABM2/ | ABM3/ | ABM2/ |
| | ABM4 | ABM4 | ABM6 | ABM5 | ABM6 | ABM5 |
| Hv 153 | ABM1/ | ABM1/ | ABM3/ | ABM2/ | ABM2/ | ABM3/ |
| | ABM4 | ABM4 | ABM6 | ABM5 | ABM5 | ABM6 |
| Hv 154 | ABM1/ | ABM1/ | ABM2/ | ABM3/ | ABM3/ | ABM2/ |
| | ABM4 | ABM4 | ABM5 | ABM6 | ABM6 | ABM5 |
| Hv 155 | ABM1/ | ABM1/ | ABM2/ | ABM3/ | ABM2/ | ABM3/ |
| | ABM4 | ABM4 | ABM5 | ABM6 | ABM5 | ABM6 |
| Hv 156 | ABM1/ | ABM1/ | ABM2/ | ABM2/ | ABM3/ | ABM3/ |
| | ABM4 | ABM4 | ABM5 | ABM5 | ABM6 | ABM6 |
| Hv 157 | ABM1/ | ABM3/ | ABM1/ | ABM3/ | ABM2/ | ABM2/ |
| | ABM4 | ABM6 | ABM4 | ABM6 | ABM5 | ABM5 |
| Hv 158 | ABM1/ | ABM3/ | ABM1/ | ABM2/ | ABM3/ | ABM2/ |
| | ABM4 | ABM6 | ABM4 | ABM5 | ABM6 | ABM5 |
| Hv 159 | ABM1/ | ABM3/ | ABM1/ | ABM2/ | ABM2/ | ABM3/ |
| | ABM4 | ABM6 | ABM4 | ABM5 | ABM5 | ABM6 |
| Hv 160 | ABM1/ | ABM2/ | ABM1/ | ABM3/ | ABM3/ | ABM2/ |
| | ABM4 | ABM5 | ABM4 | ABM6 | ABM6 | ABM5 |
| Hv 161 | ABM1/ | ABM2/ | ABM1/ | ABM3/ | ABM2/ | ABM3/ |
| | ABM4 | ABM5 | ABM4 | ABM6 | ABM5 | ABM6 |
| Hv 162 | ABM1/ | ABM2/ | ABM1/ | ABM2/ | ABM3/ | ABM3/ |
| | ABM4 | ABM5 | ABM4 | ABM5 | ABM6 | ABM6 |
| Hv 163 | ABM1/ | ABM3/ | ABM3/ | ABM1/ | ABM2/ | ABM2/ |
| | ABM4 | ABM6 | ABM6 | ABM4 | ABM5 | ABM5 |
| Hv 164 | ABM1/ | ABM3/ | ABM2/ | ABM1/ | ABM3/ | ABM2/ |
| | ABM4 | ABM6 | ABM5 | ABM4 | ABM6 | ABM5 |
| Hv 165 | ABM1/ | ABM3/ | ABM2/ | ABM1/ | ABM2/ | ABM3/ |
| | ABM4 | ABM6 | ABM5 | ABM4 | ABM5 | ABM6 |
| Hv 166 | ABM1/ | ABM2/ | ABM3/ | ABM1/ | ABM3/ | ABM2/ |
| | ABM4 | ABM5 | ABM6 | ABM4 | ABM6 | ABM5 |
| Hv 167 | ABM1/ | ABM2/ | ABM3/ | ABM1/ | ABM2/ | ABM3/ |
| | ABM4 | ABM5 | ABM6 | ABM4 | ABM5 | ABM6 |
| Hv 168 | ABM1/ | ABM2/ | ABM2/ | ABM1/ | ABM3/ | ABM3/ |
| | ABM4 | ABM5 | ABM5 | ABM4 | ABM6 | ABM6 |
| Hv 169 | ABM1/ | ABM3/ | ABM3/ | ABM2/ | ABM1/ | ABM2/ |
| | ABM4 | ABM6 | ABM6 | ABM5 | ABM4 | ABM5 |
| Hv 170 | ABM1/ | ABM3/ | ABM2/ | ABM3/ | ABM1/ | ABM2/ |
| | ABM4 | ABM6 | ABM5 | ABM6 | ABM4 | ABM5 |
| Hv 171 | ABM1/ | ABM3/ | ABM2/ | ABM2/ | ABM1/ | ABM3/ |
| | ABM4 | ABM6 | ABM5 | ABM5 | ABM4 | ABM6 |
| Hv 172 | ABM1/ | ABM2/ | ABM3/ | ABM3/ | ABM1/ | ABM2/ |
| | ABM4 | ABM5 | ABM6 | ABM6 | ABM4 | ABM5 |
| Hv 173 | ABM1/ | ABM2/ | ABM3/ | ABM2/ | ABM1/ | ABM3/ |
| | ABM4 | ABM5 | ABM6 | ABM5 | ABM4 | ABM6 |
| Hv 174 | ABM1/ | ABM2/ | ABM2/ | ABM3/ | ABM1/ | ABM3/ |
| | ABM4 | ABM5 | ABM5 | ABM6 | ABM4 | ABM6 |
| Hv 175 | ABM1/ | ABM3/ | ABM3/ | ABM2/ | ABM2/ | ABM1/ |
| | ABM4 | ABM6 | ABM6 | ABM5 | ABM5 | ABM4 |
| Hv 176 | ABM1/ | ABM3/ | ABM2/ | ABM3/ | ABM2/ | ABM1/ |
| | ABM4 | ABM6 | ABM5 | ABM6 | ABM5 | ABM4 |
| Hv 177 | ABM1/ | ABM3/ | ABM2/ | ABM2/ | ABM3/ | ABM1/ |
| | ABM4 | ABM6 | ABM5 | ABM5 | ABM6 | ABM4 |
| Hv 178 | ABM1/ | ABM2/ | ABM3/ | ABM3/ | ABM2/ | ABM1/ |
| | ABM4 | ABM5 | ABM6 | ABM6 | ABM5 | ABM4 |
| Hv 179 | ABM1/ | ABM2/ | ABM3/ | ABM2/ | ABM3/ | ABM1/ |
| | ABM4 | ABM5 | ABM6 | ABM5 | ABM6 | ABM4 |
| Hv 180 | ABM1/ | ABM2/ | ABM2/ | ABM3/ | ABM3/ | ABM1/ |
| | ABM4 | ABM5 | ABM5 | ABM6 | ABM6 | ABM4 |
| Hv 181 | ABM3/ | ABM1/ | ABM1/ | ABM3/ | ABM2/ | ABM2/ |
| | ABM6 | ABM4 | ABM4 | ABM6 | ABM5 | ABM5 |
| Hv 182 | ABM3/ | ABM1/ | ABM1/ | ABM2/ | ABM3/ | ABM2/ |
| | ABM6 | ABM4 | ABM4 | ABM5 | ABM6 | ABM5 |
| Hv 183 | ABM3/ | ABM1/ | ABM1/ | ABM2/ | ABM2/ | ABM3/ |
| | ABM6 | ABM4 | ABM4 | ABM5 | ABM5 | ABM6 |
| Hv 184 | ABM2/ | ABM1/ | ABM1/ | ABM3/ | ABM3/ | ABM2/ |
| | ABM5 | ABM4 | ABM4 | ABM6 | ABM6 | ABM5 |
| Hv 185 | ABM2/ | ABM1/ | ABM1/ | ABM3/ | ABM2/ | ABM3/ |
| | ABM5 | ABM4 | ABM4 | ABM6 | ABM5 | ABM6 |
| Hv 186 | ABM2/ | ABM1/ | ABM1/ | ABM2/ | ABM3/ | ABM3/ |
| | ABM5 | ABM4 | ABM4 | ABM5 | ABM6 | ABM6 |
| Hv 187 | ABM3/ | ABM1/ | ABM3/ | ABM1/ | ABM2/ | ABM2/ |
| | ABM6 | ABM4 | ABM6 | ABM4 | ABM5 | ABM5 |
| Hv 188 | ABM3/ | ABM1/ | ABM2/ | ABM1/ | ABM3/ | ABM2/ |
| | ABM6 | ABM4 | ABM5 | ABM4 | ABM6 | ABM5 |
| Hv 189 | ABM3/ | ABM1/ | ABM2/ | ABM1/ | ABM2/ | ABM3/ |
| | ABM6 | ABM4 | ABM5 | ABM4 | ABM5 | ABM6 |
| Hv 190 | ABM2/ | ABM1/ | ABM3/ | ABM1/ | ABM3/ | ABM2/ |
| | ABM5 | ABM4 | ABM6 | ABM4 | ABM6 | ABM5 |
| Hv 191 | ABM2/ | ABM1/ | ABM3/ | ABM1/ | ABM2/ | ABM3/ |
| | ABM5 | ABM4 | ABM6 | ABM4 | ABM5 | ABM6 |
| Hv 192 | ABM2/ | ABM1/ | ABM2/ | ABM1/ | ABM3/ | ABM3/ |
| | ABM5 | ABM4 | ABM5 | ABM4 | ABM6 | ABM6 |
| Hv 193 | ABM3/ | ABM1/ | ABM3/ | ABM2/ | ABM1/ | ABM2/ |
| | ABM6 | ABM4 | ABM6 | ABM5 | ABM4 | ABM5 |
| Hv 194 | ABM3/ | ABM1/ | ABM2/ | ABM3/ | ABM1/ | ABM2/ |
| | ABM6 | ABM4 | ABM5 | ABM6 | ABM4 | ABM5 |
| Hv 195 | ABM3/ | ABM1/ | ABM2/ | ABM2/ | ABM1/ | ABM3/ |
| | ABM6 | ABM4 | ABM5 | ABM5 | ABM4 | ABM6 |
| Hv 196 | ABM2/ | ABM1/ | ABM3/ | ABM3/ | ABM1/ | ABM2/ |
| | ABM5 | ABM4 | ABM6 | ABM6 | ABM4 | ABM5 |
| Hv 197 | ABM2/ | ABM1/ | ABM3/ | ABM2/ | ABM1/ | ABM3/ |
| | ABM5 | ABM4 | ABM6 | ABM5 | ABM4 | ABM6 |
| Hv 198 | ABM2/ | ABM1/ | ABM2/ | ABM3/ | ABM1/ | ABM3/ |
| | ABM5 | ABM4 | ABM5 | ABM6 | ABM4 | ABM6 |
| Hv 199 | ABM3/ | ABM1/ | ABM3/ | ABM2/ | ABM2/ | ABM1/ |
| | ABM6 | ABM4 | ABM6 | ABM5 | ABM5 | ABM4 |
| Hv 200 | ABM3/ | ABM1/ | ABM2/ | ABM3/ | ABM2/ | ABM1/ |
| | ABM6 | ABM4 | ABM5 | ABM6 | ABM5 | ABM4 |
| Hv 201 | ABM3/ | ABM1/ | ABM2/ | ABM2/ | ABM3/ | ABM1/ |
| | ABM6 | ABM4 | ABM5 | ABM5 | ABM6 | ABM4 |
| Hv 202 | ABM2/ | ABM1/ | ABM3/ | ABM3/ | ABM2/ | ABM1/ |
| | ABM5 | ABM4 | ABM6 | ABM6 | ABM5 | ABM4 |
| Hv 203 | ABM2/ | ABM1/ | ABM3/ | ABM2/ | ABM3/ | ABM1/ |
| | ABM5 | ABM4 | ABM6 | ABM5 | ABM6 | ABM4 |
| Hv 204 | ABM2/ | ABM1/ | ABM2/ | ABM3/ | ABM3/ | ABM1/ |
| | ABM5 | ABM4 | ABM5 | ABM6 | ABM6 | ABM4 |
| Hv 205 | ABM3/ | ABM3/ | ABM1/ | ABM1/ | ABM2/ | ABM2/ |
| | ABM6 | ABM6 | ABM4 | ABM4 | ABM5 | ABM5 |
| Hv 206 | ABM3/ | ABM2/ | ABM1/ | ABM1/ | ABM3/ | ABM2/ |
| | ABM6 | ABM5 | ABM4 | ABM4 | ABM6 | ABM5 |
| Hv 207 | ABM3/ | ABM2/ | ABM1/ | ABM1/ | ABM2/ | ABM3/ |
| | ABM6 | ABM5 | ABM4 | ABM4 | ABM5 | ABM6 |
| Hv 208 | ABM2/ | ABM3/ | ABM1/ | ABM1/ | ABM3/ | ABM2/ |
| | ABM5 | ABM6 | ABM4 | ABM4 | ABM6 | ABM5 |
| Hv 209 | ABM2/ | ABM3/ | ABM1/ | ABM1/ | ABM2/ | ABM3/ |
| | ABM5 | ABM6 | ABM4 | ABM4 | ABM5 | ABM6 |
| Hv 210 | ABM2/ | ABM2/ | ABM1/ | ABM1/ | ABM3/ | ABM3/ |
| | ABM5 | ABM5 | ABM4 | ABM4 | ABM6 | ABM6 |
| Hv 211 | ABM3/ | ABM3/ | ABM1/ | ABM2/ | ABM1/ | ABM2/ |
| | ABM6 | ABM6 | ABM4 | ABM5 | ABM4 | ABM5 |
| Hv 212 | ABM3/ | ABM2/ | ABM1/ | ABM3/ | ABM1/ | ABM2/ |
| | ABM6 | ABM5 | ABM4 | ABM6 | ABM4 | ABM5 |
| Hv 213 | ABM3/ | ABM2/ | ABM1/ | ABM2/ | ABM1/ | ABM3/ |
| | ABM6 | ABM5 | ABM4 | ABM5 | ABM4 | ABM6 |
| Hv 214 | ABM2/ | ABM3/ | ABM1/ | ABM3/ | ABM1/ | ABM2/ |
| | ABM5 | ABM6 | ABM4 | ABM6 | ABM4 | ABM5 |
| Hv 215 | ABM2/ | ABM3/ | ABM1/ | ABM2/ | ABM1/ | ABM3/ |
| | ABM5 | ABM6 | ABM4 | ABM5 | ABM4 | ABM6 |
| Hv 216 | ABM2/ | ABM2/ | ABM1/ | ABM3/ | ABM1/ | ABM3/ |
| | ABM5 | ABM5 | ABM4 | ABM6 | ABM4 | ABM6 |
| Hv 217 | ABM3/ | ABM3/ | ABM1/ | ABM2/ | ABM2/ | ABM1/ |
| | ABM6 | ABM6 | ABM4 | ABM5 | ABM5 | ABM4 |
| Hv 218 | ABM3/ | ABM2/ | ABM1/ | ABM3/ | ABM2/ | ABM1/ |
| | ABM6 | ABM5 | ABM4 | ABM6 | ABM5 | ABM4 |
| Hv 219 | ABM3/ | ABM2/ | ABM1/ | ABM2/ | ABM3/ | ABM1/ |
| | ABM6 | ABM5 | ABM4 | ABM5 | ABM6 | ABM4 |
| Hv 220 | ABM2/ | ABM3/ | ABM1/ | ABM3/ | ABM2/ | ABM1/ |
| | ABM5 | ABM6 | ABM4 | ABM6 | ABM5 | ABM4 |
| Hv 221 | ABM2/ | ABM3/ | ABM1/ | ABM2/ | ABM3/ | ABM1/ |
| | ABM5 | ABM6 | ABM4 | ABM5 | ABM6 | ABM4 |
| Hv 222 | ABM2/ | ABM2/ | ABM1/ | ABM3/ | ABM3/ | ABM1/ |
| | ABM5 | ABM5 | ABM4 | ABM6 | ABM6 | ABM4 |
| Hv 223 | ABM3/ | ABM3/ | ABM2/ | ABM1/ | ABM1/ | ABM2/ |
| | ABM6 | ABM6 | ABM5 | ABM4 | ABM4 | ABM5 |
| Hv 224 | ABM3/ | ABM2/ | ABM3/ | ABM1/ | ABM1/ | ABM2/ |
| | ABM6 | ABM5 | ABM6 | ABM4 | ABM4 | ABM5 |
| Hv 225 | ABM3/ | ABM2/ | ABM2/ | ABM1/ | ABM1/ | ABM3/ |
| | ABM6 | ABM5 | ABM5 | ABM4 | ABM4 | ABM6 |
| Hv 226 | ABM2/ | ABM3/ | ABM3/ | ABM1/ | ABM1/ | ABM2/ |
| | ABM5 | ABM6 | ABM6 | ABM4 | ABM4 | ABM5 |
| Hv 227 | ABM2/ | ABM3/ | ABM2/ | ABM1/ | ABM1/ | ABM3/ |
| | ABM5 | ABM6 | ABM5 | ABM4 | ABM4 | ABM6 |
| Hv 228 | ABM2/ | ABM2/ | ABM3/ | ABM1/ | ABM1/ | ABM3/ |
| | ABM5 | ABM5 | ABM6 | ABM4 | ABM4 | ABM6 |
| Hv 229 | ABM3/ | ABM3/ | ABM2/ | ABM1/ | ABM2/ | ABM1/ |
| | ABM6 | ABM6 | ABM5 | ABM4 | ABM5 | ABM4 |
| Hv 230 | ABM3/ | ABM2/ | ABM3/ | ABM1/ | ABM2/ | ABM1/ |
| | ABM6 | ABM5 | ABM6 | ABM4 | ABM5 | ABM4 |
| Hv 231 | ABM3/ | ABM2/ | ABM2/ | ABM1/ | ABM3/ | ABM1/ |
| | ABM6 | ABM5 | ABM5 | ABM4 | ABM6 | ABM4 |
| Hv 232 | ABM2/ | ABM3/ | ABM3/ | ABM1/ | ABM2/ | ABM1/ |
| | ABM5 | ABM6 | ABM6 | ABM4 | ABM5 | ABM4 |
| Hv 233 | ABM2/ | ABM3/ | ABM2/ | ABM1/ | ABM3/ | ABM1/ |
| | ABM5 | ABM6 | ABM5 | ABM4 | ABM6 | ABM4 |
| Hv 234 | ABM2/ | ABM2/ | ABM3/ | ABM1/ | ABM3/ | ABM1/ |
| | ABM5 | ABM5 | ABM6 | ABM4 | ABM6 | ABM4 |
| Hv 235 | ABM3/ | ABM3/ | ABM2/ | ABM2/ | ABM1/ | ABM1/ |
| | ABM6 | ABM6 | ABM5 | ABM5 | ABM4 | ABM4 |
| Hv 236 | ABM3/ | ABM2/ | ABM3/ | ABM2/ | ABM1/ | ABM1/ |
| | ABM6 | ABM5 | ABM6 | ABM5 | ABM4 | ABM4 |
| Hv 237 | ABM3/ | ABM2/ | ABM2/ | ABM3/ | ABM1/ | ABM1/ |
| | ABM6 | ABM5 | ABM5 | ABM6 | ABM4 | ABM4 |
| Hv 238 | ABM2/ | ABM3/ | ABM3/ | ABM2/ | ABM1/ | ABM1/ |
| | ABM5 | ABM6 | ABM6 | ABM5 | ABM4 | ABM4 |
| Hv 239 | ABM2/ | ABM3/ | ABM2/ | ABM3/ | ABM1/ | ABM1/ |
| | ABM5 | ABM6 | ABM5 | ABM6 | ABM4 | ABM4 |
| Hv 240 | ABM2/ | ABM2/ | ABM3/ | ABM3/ | ABM1/ | ABM1/ |
| | ABM5 | ABM5 | ABM6 | ABM6 | ABM4 | ABM4 |
| Hv 241 | ABM1/ | ABM1/ | ABM3/ | ABM2/ | ABM2/ | ABM2/ |
| | ABM4 | ABM4 | ABM6 | ABM5 | ABM5 | ABM5 |
| Hv 242 | ABM1/ | ABM1/ | ABM2/ | ABM3/ | ABM2/ | ABM2/ |
| | ABM4 | ABM4 | ABM5 | ABM6 | ABM5 | ABM5 |
| Hv 243 | ABM1/ | ABM1/ | ABM2/ | ABM2/ | ABM3/ | ABM2/ |
| | ABM4 | ABM4 | ABM5 | ABM5 | ABM6 | ABM5 |
| Hv 244 | ABM1/ | ABM1/ | ABM2/ | ABM2/ | ABM2/ | ABM3/ |
| | ABM4 | ABM4 | ABM5 | ABM5 | ABM5 | ABM6 |
| Hv 245 | ABM1/ | ABM3/ | ABM1/ | ABM2/ | ABM2/ | ABM2/ |
| | ABM4 | ABM6 | ABM4 | ABM5 | ABM5 | ABM5 |
| Hv 246 | ABM1/ | ABM2/ | ABM1/ | ABM3/ | ABM2/ | ABM2/ |
| | ABM4 | ABM5 | ABM4 | ABM6 | ABM5 | ABM5 |
| Hv 247 | ABM1/ | ABM2/ | ABM1/ | ABM2/ | ABM3/ | ABM2/ |
| | ABM4 | ABM5 | ABM4 | ABM5 | ABM6 | ABM5 |
| Hv 248 | ABM1/ | ABM2/ | ABM1/ | ABM2/ | ABM2/ | ABM3/ |
| | ABM4 | ABM5 | ABM4 | ABM5 | ABM5 | ABM6 |
| Hv 249 | ABM1/ | ABM3/ | ABM2/ | ABM1/ | ABM2/ | ABM2/ |
| | ABM4 | ABM6 | ABM5 | ABM4 | ABM5 | ABM5 |
| Hv 250 | ABM1/ | ABM2/ | ABM3/ | ABM1/ | ABM2/ | ABM2/ |
| | ABM4 | ABM5 | ABM6 | ABM4 | ABM5 | ABM5 |
| Hv 251 | ABM1/ | ABM2/ | ABM2/ | ABM1/ | ABM3/ | ABM2/ |
| | ABM4 | ABM5 | ABM5 | ABM4 | ABM6 | ABM5 |
| Hv 252 | ABM1/ | ABM2/ | ABM2/ | ABM1/ | ABM2/ | ABM3/ |
| | ABM4 | ABM5 | ABM5 | ABM4 | ABM5 | ABM6 |
| Hv 253 | ABM1/ | ABM3/ | ABM2/ | ABM2/ | ABM1/ | ABM2/ |
| | ABM4 | ABM6 | ABM5 | ABM5 | ABM4 | ABM5 |
| Hv 254 | ABM1/ | ABM2/ | ABM3/ | ABM2/ | ABM1/ | ABM2/ |
| | ABM4 | ABM5 | ABM6 | ABM5 | ABM4 | ABM5 |
| Hv 255 | ABM1/ | ABM2/ | ABM2/ | ABM3/ | ABM1/ | ABM2/ |
| | ABM4 | ABM5 | ABM5 | ABM6 | ABM4 | ABM5 |
| Hv 256 | ABM1/ | ABM2/ | ABM2/ | ABM2/ | ABM1/ | ABM3/ |
| | ABM4 | ABM5 | ABM5 | ABM5 | ABM4 | ABM6 |
| Hv 257 | ABM1/ | ABM3/ | ABM2/ | ABM2/ | ABM2/ | ABM1/ |
| | ABM4 | ABM6 | ABM5 | ABM5 | ABM5 | ABM4 |
| Hv 258 | ABM1/ | ABM2/ | ABM3/ | ABM2/ | ABM2/ | ABM1/ |
| | ABM4 | ABM5 | ABM6 | ABM5 | ABM5 | ABM4 |
| Hv 259 | ABM1/ | ABM2/ | ABM2/ | ABM3/ | ABM2/ | ABM1/ |
| | ABM4 | ABM5 | ABM5 | ABM6 | ABM5 | ABM4 |
| Hv 260 | ABM1/ | ABM2/ | ABM2/ | ABM2/ | ABM3/ | ABM1/ |
| | ABM4 | ABM5 | ABM5 | ABM5 | ABM6 | ABM4 |
| Hv 261 | ABM3/ | ABM1/ | ABM1/ | ABM2/ | ABM2/ | ABM2/ |
| | ABM6 | ABM4 | ABM4 | ABM5 | ABM5 | ABM5 |
| Hv 262 | ABM2/ | ABM1/ | ABM1/ | ABM3/ | ABM2/ | ABM2/ |
| | ABM5 | ABM4 | ABM4 | ABM6 | ABM5 | ABM5 |
| Hv 263 | ABM2/ | ABM1/ | ABM1/ | ABM2/ | ABM3/ | ABM2/ |
| | ABM5 | ABM4 | ABM4 | ABM5 | ABM6 | ABM5 |
| Hv 264 | ABM2/ | ABM1/ | ABM1/ | ABM2/ | ABM2/ | ABM3/ |
| | ABM5 | ABM4 | ABM4 | ABM5 | ABM5 | ABM6 |
| Hv 265 | ABM3/ | ABM1/ | ABM2/ | ABM1/ | ABM2/ | ABM2/ |
| | ABM6 | ABM4 | ABM5 | ABM4 | ABM5 | ABM5 |
| Hv 266 | ABM2/ | ABM1/ | ABM3/ | ABM1/ | ABM2/ | ABM2/ |
| | ABM5 | ABM4 | ABM6 | ABM4 | ABM5 | ABM5 |
| Hv 267 | ABM2/ | ABM1/ | ABM2/ | ABM1/ | ABM3/ | ABM2/ |
| | ABM5 | ABM4 | ABM5 | ABM4 | ABM6 | ABM5 |
| Hv 268 | ABM2/ | ABM1/ | ABM2/ | ABM1/ | ABM2/ | ABM3/ |
| | ABM5 | ABM4 | ABM5 | ABM4 | ABM5 | ABM6 |
| Hv 269 | ABM3/ | ABM1/ | ABM2/ | ABM2/ | ABM1/ | ABM2/ |
| | ABM6 | ABM4 | ABM5 | ABM5 | ABM4 | ABM5 |
| Hv 270 | ABM2/ | ABM1/ | ABM3/ | ABM2/ | ABM1/ | ABM2/ |
| | ABM5 | ABM4 | ABM6 | ABM5 | ABM4 | ABM5 |
| Hv 271 | ABM2/ | ABM1/ | ABM2/ | ABM3/ | ABM1/ | ABM2/ |
| | ABM5 | ABM4 | ABM5 | ABM6 | ABM4 | ABM5 |
| Hv 272 | ABM2/ | ABM1/ | ABM2/ | ABM2/ | ABM1/ | ABM3/ |
| | ABM5 | ABM4 | ABM5 | ABM5 | ABM4 | ABM6 |
| Hv 273 | ABM3/ | ABM1/ | ABM2/ | ABM2/ | ABM2/ | ABM1/ |
| | ABM6 | ABM4 | ABM5 | ABM5 | ABM5 | ABM4 |
| Hv 274 | ABM2/ | ABM1/ | ABM3/ | ABM2/ | ABM2/ | ABM1/ |
| | ABM5 | ABM4 | ABM6 | ABM5 | ABM5 | ABM4 |
| Hv 275 | ABM2/ | ABM1/ | ABM2/ | ABM3/ | ABM2/ | ABM1/ |
| | ABM5 | ABM4 | ABM5 | ABM6 | ABM5 | ABM4 |
| Hv 276 | ABM2/ | ABM1/ | ABM2/ | ABM2/ | ABM3/ | ABM1/ |
| | ABM5 | ABM4 | ABM5 | ABM5 | ABM6 | ABM4 |
| Hv 277 | ABM3/ | ABM2/ | ABM1/ | ABM1/ | ABM2/ | ABM2/ |
| | ABM6 | ABM5 | ABM4 | ABM4 | ABM5 | ABM5 |
| Hv 278 | ABM2/ | ABM3/ | ABM1/ | ABM1/ | ABM2/ | ABM2/ |
| | ABM5 | ABM6 | ABM4 | ABM4 | ABM5 | ABM5 |
| Hv 279 | ABM2/ | ABM2/ | ABM1/ | ABM1/ | ABM3/ | ABM2/ |
| | ABM5 | ABM5 | ABM4 | ABM4 | ABM6 | ABM5 |
| Hv 280 | ABM2/ | ABM2/ | ABM1/ | ABM1/ | ABM2/ | ABM3/ |
| | ABM5 | ABM5 | ABM4 | ABM4 | ABM5 | ABM6 |
| Hv 281 | ABM3/ | ABM2/ | ABM1/ | ABM2/ | ABM1/ | ABM2/ |
| | ABM6 | ABM5 | ABM4 | ABM5 | ABM4 | ABM5 |
| Hv 282 | ABM2/ | ABM3/ | ABM1/ | ABM2/ | ABM1/ | ABM2/ |
| | ABM5 | ABM6 | ABM4 | ABM5 | ABM4 | ABM5 |
| Hv 283 | ABM2/ | ABM2/ | ABM1/ | ABM3/ | ABM1/ | ABM2/ |
| | ABM5 | ABM5 | ABM4 | ABM6 | ABM4 | ABM5 |
| Hv 284 | ABM2/ | ABM2/ | ABM1/ | ABM2/ | ABM1/ | ABM3/ |
| | ABM5 | ABM5 | ABM4 | ABM5 | ABM4 | ABM6 |
| Hv 285 | ABM3/ | ABM2/ | ABM1/ | ABM2/ | ABM2/ | ABM1/ |
| | ABM6 | ABM5 | ABM4 | ABM5 | ABM5 | ABM4 |
| Hv 286 | ABM2/ | ABM3/ | ABM1/ | ABM2/ | ABM2/ | ABM1/ |
| | ABM5 | ABM6 | ABM4 | ABM5 | ABM5 | ABM4 |
| Hv 287 | ABM2/ | ABM2/ | ABM1/ | ABM3/ | ABM2/ | ABM1/ |
| | ABM5 | ABM5 | ABM4 | ABM6 | ABM5 | ABM4 |
| Hv 288 | ABM2/ | ABM2/ | ABM1/ | ABM2/ | ABM3/ | ABM1/ |
| | ABM5 | ABM5 | ABM4 | ABM5 | ABM6 | ABM4 |
| Hv 289 | ABM3/ | ABM2/ | ABM2/ | ABM1/ | ABM1/ | ABM2/ |
| | ABM6 | ABM5 | ABM5 | ABM4 | ABM4 | ABM5 |
| Hv 290 | ABM2/ | ABM3/ | ABM2/ | ABM1/ | ABM1/ | ABM2/ |
| | ABM5 | ABM6 | ABM5 | ABM4 | ABM4 | ABM5 |
| Hv 291 | ABM2/ | ABM2/ | ABM3/ | ABM1/ | ABM1/ | ABM2/ |
| | ABM5 | ABM5 | ABM6 | ABM4 | ABM4 | ABM5 |
| Hv 292 | ABM2/ | ABM2/ | ABM2/ | ABM1/ | ABM1/ | ABM3/ |
| | ABM5 | ABM5 | ABM5 | ABM4 | ABM4 | ABM6 |
| Hv 293 | ABM3/ | ABM2/ | ABM2/ | ABM1/ | ABM2/ | ABM1/ |
| | ABM6 | ABM5 | ABM5 | ABM4 | ABM5 | ABM4 |
| Hv 294 | ABM2/ | ABM3/ | ABM2/ | ABM1/ | ABM2/ | ABM1/ |
| | ABM5 | ABM6 | ABM5 | ABM4 | ABM5 | ABM4 |
| Hv 295 | ABM2/ | ABM2/ | ABM3/ | ABM1/ | ABM2/ | ABM1/ |
| | ABM5 | ABM5 | ABM6 | ABM4 | ABM5 | ABM4 |
| Hv 296 | ABM2/ | ABM2/ | ABM2/ | ABM1/ | ABM3/ | ABM1/ |
| | ABM5 | ABM5 | ABM5 | ABM4 | ABM6 | ABM4 |
| Hv 297 | ABM3/ | ABM2/ | ABM2/ | ABM2/ | ABM1/ | ABM1/ |
| | ABM6 | ABM5 | ABM5 | ABM5 | ABM4 | ABM4 |
| Hv 298 | ABM2/ | ABM3/ | ABM2/ | ABM2/ | ABM1/ | ABM1/ |
| | ABM5 | ABM6 | ABM5 | ABM5 | ABM4 | ABM4 |
| Hv 299 | ABM2/ | ABM2/ | ABM3/ | ABM2/ | ABM1/ | ABM1/ |
| | ABM5 | ABM5 | ABM6 | ABM5 | ABM4 | ABM4 |
| Hv 300 | ABM2/ | ABM2/ | ABM2/ | ABM3/ | ABM1/ | ABM1/ |
| | ABM5 | ABM5 | ABM5 | ABM6 | ABM4 | ABM4 |
| Hv 301 | ABM1/ | ABM3/ | ABM2/ | ABM2/ | ABM2/ | ABM2/ |
| | ABM4 | ABM6 | ABM5 | ABM5 | ABM5 | ABM5 |
| Hv 302 | ABM1/ | ABM2/ | ABM3/ | ABM2/ | ABM2/ | ABM2/ |
| | ABM4 | ABM5 | ABM6 | ABM5 | ABM5 | ABM5 |
| Hv 303 | ABM1/ | ABM2/ | ABM2/ | ABM3/ | ABM2/ | ABM2/ |
| | ABM4 | ABM5 | ABM5 | ABM6 | ABM5 | ABM5 |
| Hv 304 | ABM1/ | ABM2/ | ABM2/ | ABM2/ | ABM3/ | ABM2/ |
| | ABM4 | ABM5 | ABM5 | ABM5 | ABM6 | ABM5 |
| Hv 305 | ABM1/ | ABM2/ | ABM2/ | ABM2/ | ABM2/ | ABM3/ |
| | ABM4 | ABM5 | ABM5 | ABM5 | ABM5 | ABM6 |
| Hv 306 | ABM3/ | ABM1/ | ABM2/ | ABM2/ | ABM2/ | ABM2/ |
| | ABM6 | ABM4 | ABM5 | ABM5 | ABM5 | ABM5 |
| Hv 307 | ABM2/ | ABM1/ | ABM3/ | ABM2/ | ABM2/ | ABM2/ |
| | ABM5 | ABM4 | ABM6 | ABM5 | ABM5 | ABM5 |
| Hv 308 | ABM2/ | ABM1/ | ABM2/ | ABM3/ | ABM2/ | ABM2/ |
| | ABM5 | ABM4 | ABM5 | ABM6 | ABM5 | ABM5 |
| Hv 309 | ABM2/ | ABM1/ | ABM2/ | ABM2/ | ABM3/ | ABM2/ |
| | ABM5 | ABM4 | ABM5 | ABM5 | ABM6 | ABM5 |
| Hv 310 | ABM2/ | ABM1/ | ABM2/ | ABM2/ | ABM2/ | ABM3/ |
| | ABM5 | ABM4 | ABM5 | ABM5 | ABM5 | ABM6 |
| Hv 311 | ABM3/ | ABM2/ | ABM1/ | ABM2/ | ABM2/ | ABM2/ |
| | ABM6 | ABM5 | ABM4 | ABM5 | ABM5 | ABM5 |
| Hv 312 | ABM2/ | ABM3/ | ABM1/ | ABM2/ | ABM2/ | ABM2/ |
| | ABM5 | ABM6 | ABM4 | ABM5 | ABM5 | ABM5 |
| Hv 313 | ABM2/ | ABM2/ | ABM1/ | ABM3/ | ABM2/ | ABM2/ |
| | ABM5 | ABM5 | ABM4 | ABM6 | ABM5 | ABM5 |
| Hv 314 | ABM2/ | ABM2/ | ABM1/ | ABM2/ | ABM3/ | ABM2/ |
| | ABM5 | ABM5 | ABM4 | ABM5 | ABM6 | ABM5 |
| Hv 315 | ABM2/ | ABM2/ | ABM1/ | ABM2/ | ABM2/ | ABM3/ |
| | ABM5 | ABM5 | ABM4 | ABM5 | ABM5 | ABM6 |
| Hv 316 | ABM3/ | ABM2/ | ABM2/ | ABM1/ | ABM2/ | ABM2/ |
| | ABM6 | ABM5 | ABM5 | ABM4 | ABM5 | ABM5 |
| Hv 317 | ABM2/ | ABM3/ | ABM2/ | ABM1/ | ABM2/ | ABM2/ |
| | ABM5 | ABM6 | ABM5 | ABM4 | ABM5 | ABM5 |
| Hv 318 | ABM2/ | ABM2/ | ABM3/ | ABM1/ | ABM2/ | ABM2/ |
| | ABM5 | ABM5 | ABM6 | ABM4 | ABM5 | ABM5 |
| Hv 319 | ABM2/ | ABM2/ | ABM2/ | ABM1/ | ABM3/ | ABM2/ |
| | ABM5 | ABM5 | ABM5 | ABM4 | ABM6 | ABM5 |
| Hv 320 | ABM2/ | ABM2/ | ABM2/ | ABM1/ | ABM2/ | ABM3/ |
| | ABM5 | ABM5 | ABM5 | ABM4 | ABM5 | ABM6 |
| Hv 321 | ABM3/ | ABM2/ | ABM2/ | ABM2/ | ABM1/ | ABM2/ |
| | ABM6 | ABM5 | ABM5 | ABM5 | ABM4 | ABM5 |
| Hv 322 | ABM2/ | ABM3/ | ABM2/ | ABM2/ | ABM1/ | ABM2/ |
| | ABM5 | ABM6 | ABM5 | ABM5 | ABM4 | ABM5 |
| Hv 323 | ABM2/ | ABM2/ | ABM3/ | ABM2/ | ABM1/ | ABM2/ |
| | ABM5 | ABM5 | ABM6 | ABM5 | ABM4 | ABM5 |
| Hv 324 | ABM2/ | ABM2/ | ABM2/ | ABM3/ | ABM1/ | ABM2/ |
| | ABM5 | ABM5 | ABM5 | ABM6 | ABM4 | ABM5 |
| Hv 325 | ABM2/ | ABM2/ | ABM2/ | ABM2/ | ABM1/ | ABM3/ |
| | ABM5 | ABM5 | ABM5 | ABM5 | ABM4 | ABM6 |
| Hv 326 | ABM3/ | ABM2/ | ABM2/ | ABM2/ | ABM2/ | ABM1/ |
| | ABM6 | ABM5 | ABM5 | ABM5 | ABM5 | ABM4 |
| Hv 327 | ABM2/ | ABM3/ | ABM2/ | ABM2/ | ABM2/ | ABM1/ |
| | ABM5 | ABM6 | ABM5 | ABM5 | ABM5 | ABM4 |
| Hv 328 | ABM2/ | ABM2/ | ABM3/ | ABM2/ | ABM2/ | ABM1/ |
| | ABM5 | ABM5 | ABM6 | ABM5 | ABM5 | ABM4 |
| Hv 329 | ABM2/ | ABM2/ | ABM2/ | ABM3/ | ABM2/ | ABM1/ |
| | ABM5 | ABM5 | ABM5 | ABM6 | ABM5 | ABM4 |
| Hv 330 | ABM2/ | ABM2/ | ABM2/ | ABM2/ | ABM3/ | ABM1/ |
| | ABM5 | ABM5 | ABM5 | ABM5 | ABM6 | ABM4 |

### 7.6. CD2 ABMs

### 7.6.1. Immunoglobulin-Based CD2 ABMs

A first MBM (e.g., a BBM) can comprise a CD2 ABM which is an anti-CD2 antibody or an antigen-binding domain thereof. Exemplary anti-CD2 antibodies are known (see, e.g., U.S. Pat. No. 6,849,258, CN102827281A, US 2003/0139579 A1, and U.S. Pat. No. 5,795,572). Table 11A and Table 11B provide exemplary CDR, VH, and VL sequences that can be included in anti-CD2 antibodies or antigen-binding fragments thereof, for use in MBMs of the disclosure.

TABLE 11A
Immunoglobulin Based CD2 Binders

| | | | SEQ ID |
|---|---|---|---|
| Name | Domain | Sequence | NO: |
| CD2-1 | CDR-H1 | EYYMY (Rat Lo-CD2a = BTI-322 from FIG. 33 of USP | 312 |
| | | 6,849,258) | |
| CD2-1 | CDR-H2 | RIDPEDGSIDYVEKFKK (Rat Lo-CD2a = BTI-322 from FIG. | 313 |
| | | 33 of USP 6,849,258) | |
| CD2-1 | CDR-H3 | GKFNYRFAY (Rat Lo-CD2a = BTI-322 from FIG. 33 of USP | 314 |
| | | 6,849,258) | |
| CD2-1 | CDR-L1 | RSSQSLLHSSGNTYLN (Rat Lo-CD2a = BTI-322 from FIG. | 315 |
| | | 31 of USP 6,849,258) | |
| CD2-1 | CDR-L2 | LVSKLES (Rat Lo-CD2a = BTI-322 from FIG. 31 of USP | 316 |
| | | 6,849,258) | |
| CD2-1 | CDR-L3 | QFTHYPYT (Rat Lo-CD2a = BTI-322 from FIG. 31 of USP | 317 |
| | | 6,849,258) | |
| CD2-1 | VH | EVQLQQSGPELQRPGASVKLSCKASGYIFTEYYMYWVKQR | 318 |
| | | PKQGLELVGRIDPEDGSIDYVEKFKKKATLTADTSSNTAYM | |
| | | QLSSLTSEDTATYFCARGKFNYRFAYWGQGTLVTVSS | |
| | | (SEQ ID NO: 100 of USP 6,849,258) | |
| CD2-1 | VL | DVVLTQTPPTLLATIGQSVSISCRSSQSLLHSSGNTYLNWLL | 319 |
| | | QRTGQSPQPLIYLVSKLESGVPNRFSGSGSGTDFTLKISGV | |
| | | EAEDLGVYYCMQFTHYPYTFGAGTKLELK (Rat Lo-CD2a Vk | |
| | | from SEQ ID NO: 92, without signal sequence | |
| | | as shown in FIG. 31 of USP 6,849,258) | |
| hu1CD2-1 | VH | QVQLVQSGAEVKKPGASVKVSCKASGYTFTEYYMYWVRQ | 320 |
| | | APGQGLELMGRIDPEDGSIDYVEKFKKKVTLTADTSSSTAY | |
| | | MELSSLTSDDTAVYYCARGKFNYRFAYWGQGTLVTVSS | |
| | | (SEQ ID NO: 101 of USP 6,849,258) | |
| | VL | DVVMTQSPPSLLVTLGQPASISCRSSQSLLHSSGNTYLNWL | 321 |
| | | LQRPGQSPQPLIYLVSKLESGVPDRFSGSGSGTDFTLKISG | |
| | | VEAEDVGVYYCMQFTHYPYTFGQGTKLEIK (SEQ ID NO: 96 | |
| | | of USP 6,849,258) | |
| hu2CD2-1 | VH | EVQLQQSGPELQRPGASVKLSCKASGYIFTEYYMYWVKQR | 318 |
| | | PKQGLELVGRIDPEDGSIDYVEKFKKKATLTADTSSNTAYM | |
| | | QLSSLTSEDTATYFCARGKFNYRFAYWGQGTLVTVSS (Vh | |
| | | of MEDI-507; SEQ ID NO: 105 of USP 6,849,258) | |
| | VL | DVVMTQSPPSLLVTLGQPASISCRSSQSLLHSSGNTYLNWL | 321 |
| | | LQRPGQSPQPLIYLVSKLESGVPDRFSGSGSGTDFTLKISG | |
| | | VEAEDVGVYYCMQFTHYPYTFGQGTKLEIK (SEQ ID NO: 96 | |
| | | of USP 6,849,258)(same as hu1CD2-1) | |

TABLE 11B
CD2 Binders

| Binder | Domain | Sequence | SEQ ID NO: |
|---|---|---|---|
| CD2_2 | CDR-H1 | GYIFTEYYMY | 1324 |
| | (Combined Kabat + | | |
| | Chothia) | | |
| | CDR-H2 | RIDPEDGSIDYVEKFKK | 1325 |
| | (Combined Kabat + | | |
| | Chothia) | | |
| | CDR-H3 | GKFNYRFAY | 1326 |
| | (Combined Kabat + | | |
| | Chothia) | | |
| | CDR-H1 (Kabat) | EYYMY | 1327 |
| | CDR-H2 (Kabat) | RIDPEDGSIDYVEKFKK | 1325 |
| | CDR-H3 (Kabat) | GKFNYRFAY | 1326 |
| | CDR-H1 (Chothia) | GYIFTEY | 1328 |
| | CDR-H2 (Chothia) | DPEDGS | 1329 |
| | CDR-H3 (Chothia) | GKFNYRFAY | 1326 |
| | CDR-H1 (IMGT) | GYIFTEYY | 1330 |
| | CDR-H2 (IMGT) | IDPEDGSI | 1331 |
| | CDR-H3 (IMGT) | ARGKFNYRFAY | 1332 |
| | VH | QVQLVQSGAEVQRPGASVKVSCKASGYIFTEYYM | 1333 |
| | | YWWRQAPGQGLELVGRIDPEDGSIDYVEKFKKKVT | |
| | | LTADTSSSTAYMELSSLTSDDTAVYYCARGKFNYR | |
| | | FAYWGQGTLVTVSS | |
| | CDR-L1 (Combined | RSSQSLLHSSGNTYLN | 1334 |
| | Kabat + Chothia) | | |
| | CDR-L2 (Combined | LVSKLES | 1335 |
| | Kabat + Chothia) | | |
| | CDR-L3 (Combined | MQFTHYPYT | 1336 |
| | Kabat + Chothia) | | |
| | CDR-L1 (Kabat) | RSSQSLLHSSGNTYLN | 1334 |
| | CDR-L2 (Kabat) | LVSKLES | 1335 |
| | CDR-L3 (Kabat) | MQFTHYPYT | 1336 |
| | CDR-L1 (Chothia) | SQSLLHSSGNTY | 1337 |
| | CDR-L2 (Chothia) | LVS | 1338 |
| | CDR-L3 (Chothia) | FTHYPY | 1339 |
| | CDR-L1 (IMGT) | QSLLHSSGNTY | 1340 |
| | CDR-L2 (IMGT) | LVS | 1338 |
| | CDR-L3 (IMGT) | MQFTHYPYT | 1336 |
| | VL | DWMTQSPPSLLVTLGQPASISCRSSQSLLHSSGN | 321 |
| | | TYLNWLLQRPGQSPQPLIYLVSKLESGVPDRFSGS | |
| | | GSGTDFTLKiSGVEAEDVGVYYCMQFTHYPYTFG | |
| | | QGTKLEiK | |

In some embodiments, a CD2 ABM comprises the CDR sequences of CD2-1. In some embodiments, a CD2 ABM comprises the heavy and light chain variable sequences of CD2-1. In some embodiments, a CD2 ABM comprises the heavy and light chain variable sequences of hu1CD2-1. In some embodiments, a CD2 ABM comprises the heavy and light chain variable sequences of hu2CD2-1.

In other embodiments, a CD2 ABM can comprise the CDR sequences of antibody 9D1 produced by the hybridoma deposited with the Chinese Culture Collection Committee General Microbiology Center on May 16, 2012 with accession no. CGMCC 6132, and which is described in CN102827281A. In other embodiments, a CD2 ABM can comprise the CDR sequences of antibody LO-CD2b produced by the hybridoma deposited with the American Type Culture Collection on Jun. 22, 1999 with accession no. PTA-802, and which is described in US 2003/0139579 A1. In yet other embodiments, a CD2 ABM can comprise the CDR sequences of the CD2 SFv-Ig produced by expression of the construct cloned in the recombinant E. coli deposited with the ATCC on Apr. 9, 1993 with accession no. 69277, and which is described in U.S. Pat. No. 5,795,572.

In other embodiments, a CD2 ABM can comprise the VH and VL sequences of antibody 9D1. In other embodiments, a CD2 ABM can comprise the VH and VL sequences of antibody LO-CD2b. In yet other embodiments, a CD2 ABM can comprise the VH and VL sequences of the CD2 SFv-Ig produced by expression of the construct cloned in the recombinant E. coli having ATCC accession no. 69277.

In some embodiments, a CD2 ABM comprises Kabat CDR-H1, CDR-H2, CDR-H3, CDR-L1, CDR-L2, and CDR-L3 sequences of CD2_2 as set forth in Table 11B. In some embodiments, a CD2 ABM comprises Chothia CDR-H1, CDR-H2, CDR-H3, CDR-L1, CDR-L2, and CDR-L3 sequences of CD2_2 as set forth in Table 11B. In some embodiments, a CD2 ABM comprises IMGT CDR-H1, CDR-H2, CDR-H3, CDR-L1, CDR-L2, and CDR-L3 sequences of CD2_2 as set forth in Table 11B. In some embodiments, a CD2 ABM comprises combined Kabat+Chothia CDR-H1, CDR-H2, CDR-H3, CDR-L1, CDR-L2, and CDR-L3 sequences of CD2_2 as set forth in Table 11B. In some embodiments, a CD2 ABM comprises VH and/or VL sequences of CD2_2 as set forth in Table 11B.

### 7.6.2. CD58-based CD2 ABMs

A first MBM (e.g., a BBM) can comprise a CD2 ABM which is a ligand. The CD2 ABM specifically binds to human CD2, whose natural ligand is CD58, also known as LFA-3. CD58/LFA-3 proteins are glycoproteins that are expressed on the surfaces of a variety of cell types (Dustin et al., 1991, Annu. Rev. Immunol. 9:27) and play roles in mediating T-cell interactions with APCs in both antigen-dependent and antigen-independent manners (Wallner et al., 1987, J. Exp. Med. 166:923). Accordingly, in certain aspects, the CD2 ABM is a CD58 moiety. As used herein, a CD58 moiety comprises an amino acid sequence comprising at least 70% sequence identity to a CD2-binding portion of CD58, e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to a CD2-binding portion of CD58. The sequence of human CD58 has the Uniprot identifier P19256 (www.uniprot.org/uniprot/P19256). It has been established that CD58 fragments containing amino acid residues 30-123 of full length CD58 (i.e., the sequence designated as CD58-6 in Table 12 below) are sufficient for binding to CD2. Wang et al., 1999, Cell 97:791-803. Accordingly, in certain aspects, a CD58 moiety comprises an amino acid sequence comprising at least 70% sequence identity to amino acids 30-123 of CD58, e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to the amino acid sequence designated CD58-6.

The interactions between CD58 and CD2 have been mapped through x-ray crystallography and molecular modeling. The substitution of residues E25, K29, K30, K32, D33, K34, E37, D84 and K87 (with numbering referring to the in the mature polypeptide) reduces binding to CD2. Ikemizu et al., 1999, Proc. Natl. Acad. Sci. USA 96:4289-94. Accordingly, in some embodiments the CD58 moiety retains the wild type residues at E25, K29, K30, K32, D33, K34, E37, D84 and K87.

In contrast, the following substitutions (with numbering referring to the full length polypeptide) did not impact binding to CD2: F29S; V37K; V49Q; V86K; T113S; and L121G. Accordingly, a CD58 moiety can include one, two, three, four, five or all six of the foregoing substitutions.

In some embodiments, the CD58 moiety is engineered to include a pair of cysteine substitutions that upon recombinant expression create a disulfide bridge. Exemplary amino acid pairs that can be substituted with cysteines in order to form a disulfide bridge upon expression (with numbering referring to the full length polypeptide) are (a) a V45C substitution and a M105C substitution; (b) a V54C substitution and a G88C substitution; (c) a V45C substitution and a M114C substitution; and (d) a W56C substitution and a L900 substitution.

Exemplary CD58 moieties are provided in Table 12 below:

TABLE 12
CD58 sequences

| Name | Description | Sequence | SEQ ID NO: |
|---|---|---|---|
| CD58-1 | Full length CD58, | MVAGSDAGRALGVLSVVCLLHCFGFISCFSQQIYGVVY | 322 |
| | including signal | GNVTFHVPSNVPLKEVLWKKQKDKVAELENSEFRAFS | |
| | sequence and full | SFKNRVYLDTVSGSLTIYNLTSSDEDEYEMESPNITDTM | |
| | intracellular domain | KFFLYVLESLPSPTLTCALTNGSIEVQCMIPEHYNSHRG | |
| | (P19256) | LIMYSWDCPMEQCKRNSTSIYFKMENDLPQKIQCTLSN | |
| | | PLFNTTSSIILTTCIPSSGHSRHRYALIPIPLAVITTC | |
| | | IVLYMNGILKCDRKPDRTNSN | |
| CD58-2 | Full length CD58, | MVAGSDAGRALGVLSVVCLLHCFGFISCFSQQIYGVVY | 323 |
| | including signal | GNVTFHVPSNVPLKEVLWKKQKDKVAELENSEFRAFS | |
| | sequence and but | SFKNRVYLDTVSGSLTIYNLTSSDEDEYEMESPNITDTM | |
| | no intracellular | KFFLYVLESLPSPTLTCALTNGSIEVQCMIPEHYNSHRG | |
| | domain (P19256-2) | LIMYSWDCPMEQCKRNSTSIYFKMENDLPQKIQCTLSN | |
| | | PLFNTTSSIILTTCIPSSGHSRHRYALIPIPLAVITT | |
| | | CIVLYMNVL | |
| CD58-3 | Full length CD58, | MVAGSDAGRALGVLSVVCLLHCFGFISCFSQQIYGVVY | 324 |
| | including signal | GNVTFHVPSNVPLKEVLWKKQKDKVAELENSEFRAFS | |
| | sequence and | SFKNRVYLDTVSGSLTIYNLTSSDEDEYEMESPNITDTM | |
| | variant intracellular | KFFLYVLESLPSPTLTCALTNGSIEVQCMIPEHYNSHRG | |
| | domain (P19256-3) | LIMYSWDCPMEQCKRNSTSIYFKMENDLPQKIQCTLSN | |
| | | PLFNTTSSIILTTCIPSSGHSRHRYALIPIPLAVITTC | |
| | | IVLYMNGILKCDRKPDRTK | |
| CD58-4 | Extracellular domain | FSQQIYGVVYGNVTFHVPSNVPLKEVLWKKQKDKVAE | 325 |
| | of CD58, | LENSEFRAFSSFKNRVYLDTVSGSLTIYNLTSSDEDEYE | |
| | corresponding to | MESPNITDTMKFFLYVLESLPSPTLTCALTNGSIEVQCM | |
| | amino acids 29-215 | IPEHYNSHRGLIMYSWDCPMEQCKRNSTSIYFKMENDL | |
| | of CD58 (WT) | PQKIQCTLSNPLFNTTSSIILTTCIPSSGHSRHR | |
| CD58-5 | Extracellular domain | BSQQIYGVJYGNVTFHVPSNOPLKEVLWKKQKDK | 326 |
| | of CD58, | VAELENSEFRAFSSFKNRVYLDTUSGSLTIYNLTS | |
| | corresponding to | SDEDEYEMESPNITDXMKFFLYVZESLPSPTLTCA | |
| | amino acids 29-215 | LTNGSIEVQCMIPEHYNSHRGLIMYSWDCPMEQC | |
| | of CD58 (with | KRNSTSIYFKMENDLPQKIQCTLSNPLFNTTSSIILT | |
| | permitted | TCIPSSGHSRHR | |
| | substitutions) | B = F or S | |
| | | J = V or K | |
| | | O = V or Q | |
| | | U = V or K | |
| | | X = T or S | |
| | | Z = L or G | |
| CD58-6 | Amino acids 30-123 | SQQIYGVVYGNVTFHVPSNVPLKEVLWKKQKDKVAEL | 327 |
| | (WT) | ENSEFRAFSSFKNRVYLDTVSGSLTIYNLTSSDEDEYE | |
| | Ig-V like domain | MESPNITDTMKFFLYVLES | |
| CD58-7 | Amino acids 30-123 | SQQIYGVJYGNVTFHVPSNOPLKEVLWKKQKDKVAEL | 328 |
| | (with permitted | ENSEFRAFSSFKNRVYLDTVSGSLTIYNLTSSDEDEYE | |
| | substitutions) | MESPNITDTMKFFLYVLES | |
| | Ig-V like domain | J = V or K | |
| | | O = V or Q | |
| CD58-8 | Amino acids 30-123 | SQQIYGVVYGNVTFHCPSNVPLKEVLWKKQKDKVAEL | 329 |
| | (V45C_M105C) | ENSEFRAFSSFKNRVYLDTVSGSLTIYNLTSSDEDEYE | |
| | Ig-V like domain | CESPNITDTMKFFLYVLES | |
| CD58-9 | Amino acids 30-123 | SQQIYGVVYGNVTFHVPSNVPLKECLWKKQKDKVAEL | 330 |
| | (V54C_G88C) | ENSEFRAFSSFKNRVYLDTVSCSLTIYNLTSSDEDEYE | |
| | Ig-V like domain | MESPNITDTMKFFLYVLES | |
| CD58-10 | Amino acids 30-123 | SQQIYGVVYGNVTFHCPSNVPLKEVLWKKQKDKVAEL | 331 |
| | (V45C_M114C) | ENSEFRAFSSFKNRVYLDTVSGSLTIYNLTSSDEDEYE | |
| | Ig-V like domain | MESPNITDTCKFFLYVLES | |
| CD58-11 | Amino acids 30-123 | SQQIYGVVYGNVTFHVPSNVPLKEVLCKKQKDKVAELE | 332 |
| | (W56C_L90C) | NSEFRAFSSFKNRVYLDTVSGSCTIYNLTSSDEDEYEM | |
| | Ig-V like domain | ESPNITDTMKFFLYVLES | |

### 7.6.3. CD48-Based CD2 ABMs

A first MBM can comprise a CD2 ABM which is CD48 moiety. As used herein, a CD48 moiety comprises an amino acid sequence comprising at least 70% sequence identity to a CD2-binding portion of CD48, e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to a CD2-binding portion of CD48. The sequence of human CD48 has the Uniprot identifier P09326 (www.uniprot.org/uniprot/P09326), which includes a signal peptide (amino acids 1-26) and a GPI anchor (amino acids 221-243). In certain aspects, a CD48 moiety comprises an amino acid sequence comprising at least 70% sequence identity (e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity) to the amino acid sequence of consisting of amino acids 27-220 of Uniprot identifier P09326. Human CD48 has an Ig-like C2-type I domain (amino acids 29-127 of Uniprot identifier P09326) and a Ig-like C2 type 2 domain (amino acids 132-212 of Uniprot identifier P09326). Accordingly, in some embodiments, a CD48 moiety comprises an amino acid sequence comprising at least 70% sequence identity (e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity) to the amino acid sequence of consisting of amino acids 29-212 of Uniprot identifier P09326, to the C2-type I domain (amino acids 29-127 of Uniprot identifier P09326) and/or to the Ig-like C2 type 2 domain (amino acids 132-212 of Uniprot identifier P09326). A CD48 moiety can in some embodiments comprise one or more natural variants relative to the sequence of Uniprot identifier P09326. For example, a CD48 moiety can include a E102Q substitution. As another example, a CD48 moiety can comprise an amino acid sequence corresponding to a CD-48 isoform or a CD2 binding portion thereof, e.g., the isoform having Uniprot identifier P09326-2 or a CD2 binding portion thereof.

### 7.7. Tumor-Associated Antigen ABMs

ABM2 of the first MBMs and ABM5 of the second MBMs of the disclosure, when present, bind specifically to a tumor-associated antigen (TAA) (“TAA 1” and “TAA 2,” respectfully). In some combinations of first and second MBMs, only one of the first and second MBM has a ABM that binds to a TAA. In other combinations, both the first and second MBM has a ABM that binds to a TAA. In some of these combinations, TAA 1 and TAA 2 are the same. In other combinations of first and second MBMs in which both MBMs have a ABM that binds to a TAA, TAA 1 and TAA 2 are different. When TAA 1 and TAA 2 are the same, ABM2 and ABM5 preferably bind to different epitopes on the TAA (e.g., non-overlapping epitopes) so that the first MBM and the second MBM are able to specifically bind to the TAA simultaneously. In some embodiments, ABM2 and ABM5 are selected so that binding of the first MBM to the TAA reduces binding of the second MBM to the TAA by no more than, and in some embodiments, less than, 50% (e.g., less than 40%, less than 30%, less than 20% or less than 20%) in a competition assay such as an ELISA assay, Biacore assay, FACS assay, or another competition assay in the art.

Preferably, TAA 1 and/or TAA 2 are human TAAs. TAA 1 and/or TAA 2 may or may not be present on normal cells. In certain embodiments, TAA 1 and/or TAA 2 are preferentially expressed or upregulated on tumor cells as compared to normal cells. In other embodiments, TAA 1 and/or TAA 2 are lineage marker(s).

It is anticipated that any type of tumor and any type of TAA may be targeted by the MBMs of the disclosure. Exemplary types of cancers that may be targeted include acute lymphoblastic leukemia, acute myelogenous leukemia, biliary cancer, B-cell leukemia, B-cell lymphoma, biliary cancer, bone cancer, brain cancer, breast cancer, triple-negative breast cancer, cervical cancer, Burkitt lymphoma, chronic lymphocytic leukemia, chronic myelogenous leukemia, colorectal cancer, endometrial cancer, esophageal cancer, gall bladder cancer, gastric cancer, gastrointestinal tract cancer, glioma, hairy cell leukemia, head and neck cancer, Hodgkin's lymphoma, liver cancer, lung cancer, medullary thyroid cancer, melanoma, multiple myeloma, ovarian cancer, non-Hodgkin's lymphoma, pancreatic cancer, prostate cancer, pulmonary tract cancer, renal cancer, sarcoma, skin cancer, testicular cancer, urothelial cancer, and other urinary bladder cancers. However, the skilled artisan will realize that TAAs are known for virtually any type of cancer.

Exemplary TAAs that can be targeted by MBMs of the disclosure include ABCF1; ACVR1; ACVR1B; ACVR2; ACVR2B; ACVRL1; ADORA2A; ADRB3; Aggrecan; AGR2; AICDA; AIF1; AIG1; AKAP1; AKAP2; ALK; AMH; AMHR2; ANGPT1; ANGPT2; ANGPTL3; ANGPTL4; ANPEP; APC; APOC1; AR; AZGP1 (zinc-a-glycoprotein); B7.1; B7.2; BAD; BAFF; BAG1; BAI1; BCL2; BCL6; BDNF; BLNK; BLR1 (MDR15); BlyS; BMP1; BMP2; BMP3B (GDF10); BMP4; BMP6; BMP8; BMPR1A; BMPR1B; BMPR2; BPAG1 (plectin); BRCA1; C19orf10 (IL27w); C3; C4A; C5; C5R1; Cadherin 17; CANT1; CASP1; CASP4; CAV1; CCBP2 (D6/JAB61); CCL1 (1-309); CCL11 (eotaxin); CCL13 (MCP-4); CCL15 (MIP-1d); CCL16 (HCC-4); CCL17 (TARC); CCL18 (PARC); CCL19 (MIP-3b); CCL2 (MCP-1); MCAF; CCL20 (MIP-3a); CCL21 (MIP-2); SLC; exodus-2; CCL22 (MDC/STC-1); CCL23 (MPIF-1); CCL24 (MPIF-2/eotaxin-2); CCL25 (TECK); CCL26 (eotaxin-3); CCL27 (CTACK/ILC); CCL28; CCL3 (MIP-1a); CCL4 (MIP-1b); CCL5 (RANTES); CCL7 (MCP-3); CCL8 (mcp-2); CCNA1; CCNA2; CCND1; CCNE1; CCNE2; CCR1 (CKR1/HM145); CCR2 (mcp-1RB/RA); CCR3 (CKR3/CMKBR3); CCR4; CCR5 (CMKBR5/ChemR13); CCR6 (CMKBR6/CKR-L3/STRL22/DRY6); CCR7 (CKR7/EBI1); CCR8 (CMKBR8/TER1/CKR-L1); CCR9 (GPR-9-6); CCRL1 (VSHK1); CCRL2 (L-CCR); CD164; CD19; CD1C; CD20; CD200; CD-22; CD24; CD28; CD3; CD37; CD38; CD3E; CD3G; CD3Z; CD4; CD32b; CD40; CD40L; CD44; CD45RB; CD52; CD69; CD72; CD74; CD79A; CD79B; CD8; CD80; CD81; CD83; CD86; CD97; CD179a; CDH1 (E-cadherin); CDH10; CDH12; CDH13; CDH18; CDH19; CDH20; CDH5; CDH7; CDH8; CDH9; CDK2; CDK3; CDK4; CDK5; CDK6; CDK7; CDK9; CDKN1A (p21Wap1/Cip1); CDKN1B (p27Kip1); CDKN1C; CDKN2A (p16INK4a); CDKN2B; CDKN2C; CDKN3; CEBPB; CER1; CHGA; CHGB; Chitinase; CHST10; CKLFSF2; CKLFSF3; CKLFSF4; CKLFSF5; CKLFSF6; CKLFSF7; CKLFSF8; CLDN3; CLDN6 (claudin-6); CLDN7 (claudin-7); CLN3; CLU (clusterin); CMKLR1; CMKOR1 (RDC1); CNR1; COL18A1; COL1A1; COL4A3; COL6A1; CR2; CRP; CSF1 (M-CSF); CSF2 (GM-CSF); CSF3 (GCSF); CTLA4; CTNNB1 (b-catenin); CTSB (cathepsin B); CX3CL1 (SCYD1); CX3CR1 (V28); CXCL1 (GRO1); CXCL10(IP-10); CXCL11 (1-TAC/IP-9); CXCL12 (SDF1); CXCL13; CXCL14; CXCL16; CXCL2 (GRO2); CXCL3 (GRO3); CXCL5 (ENA-78/LIX); CXCL6 (GCP-2); CXCL9 (MIG); CXCR3 (GPR9/CKR-L2); CXCR4; CXCR6 (TYMSTR/STRL33/Bonzo); CYB5; CYC1; CYSLTR1; CGRP; C1q; C1r; C1; C4a; C4b; C2a; C2b; C3a; C3b; DAB2IP; DES; DKFZp451J0118; DNCL1; DPP4; E-selectin; E2F1; ECGF1; EDG1; EFNA1; EFNA3; EFNB2; EGF; EGFR; EGFRvIII; ELAC2; ENG; ENO1; ENO2; ENO3; EPHB4; EPO; ERBB2 (Her-2); EREG; ERK8; ESR1; ESR2; F3 (TF); Factor VII; Factor IX; Factor V; Factor Vila; Factor Factor X; Factor XII; Factor XIII; FADD; FasL; FASN; FCER1A; FCER2; Fc gamma receptor; FCGR3A; FCRL5; FGF; FGF1 (aFGF); FGF10; FGF11; FGF12; FGF12B; FGF13; FGF14; FGF16; FGF17; FGF18; FGF19; FGF2 (bFGF); FGF20; FGF21; FGF22; FGF23; FGF3 (int-2); FGF4 (HST); FGF5; FGF6 (HST-2); FGF7 (KGF); FGF8; FGF9; FGFR3; FIGF (VEGFD); FIL1 (EPSILON); FIL1 (ZETA); FLJ12584; FLJ25530; FLRT1 (fibronectin); FLT1; Folate receptor alpha; Folate receptor beta; FOS; FOSL1 (FRA-1); Fucosyl GM1; FY (DARC); GABRP (GABAa); GAGEB1; GAGEC1; GALNAC4S-65T; GATA3; GDF5; GFI1; GGT1; GM-CSF; GloboH; GNAS1; GNRH1; GPNMB; GPR2 (CCR10); GPR20; GPR31; GPR44; GPR64; GPR81 (FKSG80); GPRC5D; GRCC10 (C10); GRP; GSN (Gelsolin); GSTP1; glycoprotein (gP) IIb/IIIa; HAVCR1; HAVCR2; HDAC4; HDAC5; HDAC7A; HDAC9; Her2; HER3; HGF; HIF1A; HIP1; histamine and histamine receptors; HLA-A; HLA-DRA; HM74; HMGB1; HMOX1; HMWMAA; HUMCYT2A; ICEBERG; ICOSL; ID2; IFN-a; IFNA1; IFNA2; IFNA4; IFNA5; IFNA6; IFNA7; IFNB1; IFN-γ; IFNW1; IGBP1; IGF1; IGF1R; IGF2; IGFBP2; IGFBP3; IGFBP6; IL-1; IL-α; IL-1-β; IL10; IL10RA; IL10RB; IL11; IL11RA; IL-12; IL12A; IL12B; IL12RB1; IL12RB2; IL13; IL13RA1; IL13RA2; IL14; IL15; IL15RA; IL16; IL17; IL17B; IL17C; IL17R; IL18; IL18BP; IL18R1; IL18RAP; IL19; IL1A; IL1B; IL1F10; IL1F5; IL1F6; IL1F7; IL1F8; IL1F9; IL1HY1; IL1R1; IL1R2; IL1RAP; IL1RAPL1; IL1RAPL2; IL1RL1; IL1RL2; IL1RN; IL2; IL20; IL20RA; IL21R; IL22; IL22R; IL22RA2; IL23; IL24; IL25; IL26; IL27; IL28A; IL28B; IL29; IL2RA; IL2RB; IL2RG; IL3; IL30; IL3RA; IL4; IL4R; IL5; IL5RA; IL6; IL6R; IL6ST (glycoprotein 130); IL7; IL7R; IL8; IL8RA; IL8RB; IL8RB; IL9; IL9R; ILK; INHA; INHBA; INSL3; INSL4; IRAK1; IRAK2; ITGA1; ITGA2; ITGA3; ITGA6 (a6 integrin); ITGAV; ITGB3; ITGB4 (b 4 integrin); JAG1; JAK1; JAK3; JUN; K6HF; KAI1; KDR; KITLG; KLF5 (GC Box BP); KLF6; KLK10; KLK12; KLK13; KLK14; KLK15; KLK3; KLK4; KLK5; KLK6; KLK9; KRT1; KRT19 (Keratin 19); KRT2A; KRTHB6 (hair-specific type II keratin); L-selectin; LAMAS; LEP (leptin); Lingo-p75; Lingo-Troy; LRP6; LPS; LTA (TNF-b); LTB; LTB4R (GPR16); LTB4R2; LTBR; LY6K; LYPD8; MACMARCKS; MAG or Omgp; MAP2K7 (c-Jun); MDK; mesothelin; MIB1; midkine; MIF; MIP-2; MKI67 (Ki-67); MMP2; MMP9; MS4A1; MSMB; MT3 (metallothionectin-III); MTSS1; MUC1 (mucin); MYC; MYD88; NCK2; neurocan; NKG2D; NFKB1; NFKB2; NGF; NGFB (NGF); NGFR; NgR-Lingo; NgR-Nogo66 (Nogo); NgR-p75; NgR-Troy; NME1 (NM23A); NOX5; NPPB; NR0B1; NR0B2; NR1D1; NR1D2; NR1H2; NR1H3; NR1H4; NRII2; NRII3; NR2C1; NR2C2; NR2E1; NR2E3; NR2F1; NR2F2; NR2F6; NR3C1; NR3C2; NR4A1; NR4A2; NR4A3; NR5A1; NR5A2; NR6A1; NRP1; NRP2; NT5E; NTN4; NY-BR-1; o-acetyl-GD2; ODZ1; OPRD1; OR51E2; P2RX7; PANX3; PAP; PART1; PATE; PAWR; PCA3; PCNA; PDGFA; PDGFB; PECAM1; PF4 (CXCL4); PGE2; PGF; PGR; phosphacan; PIAS2; PIK3CG; PLAC1; plasminogen activator; PLAU (uPA); PLG; PLXDC1; polysialic acid; PPBP (CXCL7); PPID; PR1; PRKCQ; PRKD1; PRL; PROC; Protein C; PROK2; PSAP; PSCA; PTAFR; PTEN; PTGS2 (COX-2); PTN; RAC2 (p21Rac2); RAGE; RARB; RGS1; RGS13; RGS3; RNF110 (ZNF144); ROBO2; SIO0A2; SCGB1D2 (lipophilin B); SCGB2A1 (mammaglobin 2); SCGB2A2 (mammaglobin 1); SCYE1 (endothelial Monocyte-activating cytokine); SDF2; SERPINA1; SERPINA3; SERPINB5 (maspin); SERPINE1 (PAI-1); SERPINF1; SHBG; SLA2; SLC2A2; SLC33A1; SLC34A2; SLC39A6; SLC43A1; SLIT2; SLITRK6; SPP1; SPRR1B (Spr1); ST6GAL1; STAB1; STAT6; STEAP; STEAP2; substance P; TACSTD2; TB4R2; TBX21; TCP10; TDGF1; TEK; TEM1/CD248; TEM7R; TGFA; TGFB1; TGFB111; TGFB2; TGFB3; TGFBI; TGFBR1; TGFBR2; TGFBR3; TH1L; THBS1 (thrombospondin-1); THBS2; THBS4; THPO; TIE (Tie-1); TIMP3; tissue factor; TLR10; TLR2; TLR3; TLR4; TLR5; TLR6; TLR7; TLR8; TLR9; TNF; TNF-a; TNFAIP2 (B94); TNFAIP3; TNFRSF11A; TNFRSF1A; TNFRSF1B; TNFRSF21; TNFRSF5; TNFRSF6 (Fas); TNFRSF7; TNFRSF8; TNFRSF9; TNFSF10 (TRAIL); TNFSF11 (TRANCE); TNFSF12 (APO3L); TNFSF13 (April); TNFSF13B; TNFSF14 (HVEM-L); TNFSF15 (VEGI); TNFSF18; TNFSF4 (OX40 ligand); TNFSF5 (CD40 ligand); TNFSF6 (FasL); TNFSF7 (CD27 ligand); TNFSF8 (CD30 ligand); TNFSF9 (4-1BB ligand); TOLLIP; Toll-like receptors; TOP2A (topoisomerase ha); TP53; TPM1; TPM2; TRADD; TRAF1; TRAF2; TRAF3; TRAF4; TRAF5; TRAF6; TREM1; TREM2; TRPC6; TSHR; TSLP; TWEAK; thrombomodulin; thrombin; UPK2; VEGF; VEGFB; VEGFC; versican; VHL C5; VLA-4; XCL1 (lymphotactin); XCL2 (SCM-1b); XCR1 (GPRS/CCXCR1); YY1; and ZFPM2.

In some embodiments, a TAA (e.g., TAA 1 and/or TAA 2) targeted by a MBM of the disclosure is mesothelin, TSHR, CD171, CS-1, CLL-1, GD3, Tn Ag, FLT3, CD38, CD44v6, B7H3, KIT, IL-13Ra2, IL-11Ra, PSCA, PRSS21, VEGFR2, LewisY, CD24, PDGFR-beta, SSEA-4, MUC1, EGFR, NCAM, CAIX, LMP2, EphA2, fucosyl GM1, sLe, GM3, TGS5, HMWMAA, o-acetyl-GD2, GD2, folate receptor alpha, folate receptor beta, TEM1/CD248, TEM7R, CLDN6, GPRC5D, CXORF61, CD97, CD179a, ALK, polysialic acid, PLAC1, GloboH, NY-BR-1, UPK2, HAVCR1, ADRB3, PANX3, GPR20, LY6K, OR51E2, TAARP, WT1, ETV6-AML, sperm protein 17, XAGE1, Tie 2, MAD-CT-1, MAD-CT-2, Fos-related antigen 1, p53 mutant, hTERT, sarcoma translocation breakpoints, ML-IAP, ERG (TMPRSS2 ETS fusion gene), NA17, PAX3, Androgen receptor, Cyclin B1, MYCN, RhoC, CYP1B1, BORIS, SART3, PAX5, OY-TES1, LCK, AKAP-4, SSX2, CD79a, CD79b, CD72, LAIR1, FCAR, LILRA2, CD300LF, CLEC12A, BST2, EMR2, LY75, GPC3, FCRL5, IGLL1, CD19, CD20, CD30, ERBB2, ROR1, TAAG72, CD22, GD2, BCMA, gp100Tn, FAP, tyrosinase, EPCAM, CEA, Igf-I receptor, EphB2, Cadherin17, CD32b, EGFRvIII, GPNMB, GPR64, HER3, LRP6, LYPD8, NKG2D, SLC34A2, SLC39A6, SLITRK6, TACSTD2, CD123, CD33, CD138, CS1, CD133, CD52, TNFRSF13C, TNFRSF13B, CXCR4, PD-L1, LY9, CD200, FCGR2B, CD21, CD23, or CD40L.

In some combinations of first and second MBMs, TAA 1 and TAA 2 are the same, and the TAA is mesothelin, TSHR, CD171, CS-1, CLL-1, GD3, Tn Ag, FLT3, CD38, CD44v6, B7H3, KIT, IL-13Ra2, IL-11Ra, PSCA, PRSS21, VEGFR2, LewisY, CD24, PDGFR-beta, SSEA-4, MUC1, EGFR, NCAM, CAIX, LMP2, EphA2, fucosyl GM1, sLe, GM3, TGS5, HMWMAA, o-acetyl-GD2, GD2, folate receptor alpha, folate receptor beta, TEM1/CD248, TEM7R, CLDN6, GPRC5D, CXORF61, CD97, CD179a, ALK, polysialic acid, PLAC1, GloboH, NY-BR-1, UPK2, HAVCR1, ADRB3, PANX3, GPR20, LY6K, OR51E2, TAARP, WT1, ETV6-AML, sperm protein 17, XAGE1, Tie 2, MAD-CT-1, MAD-CT-2, Fos-related antigen 1, p53 mutant, hTERT, sarcoma translocation breakpoints, ML-IAP, ERG (TMPRSS2 ETS fusion gene), NA17, PAX3, Androgen receptor, Cyclin B1, MYCN, RhoC, CYP1B1, BORIS, SART3, PAX5, OY-TES1, LCK, AKAP-4, SSX2, CD79a, CD79b, CD72, LAIR1, FCAR, LILRA2, CD300LF, CLEC12A, BST2, EMR2, LY75, GPC3, FCRL5, IGLL1, CD19, CD20, CD30, ERBB2, ROR1, TAAG72, CD22, GD2, BCMA, gp100Tn, FAP, tyrosinase, EPCAM, CEA, Igf-I receptor, EphB2, Cadherin17, CD32b, EGFRvIII, GPNMB, GPR64, HER3, LRP6, LYPD8, NKG2D, SLC34A2, SLC39A6, SLITRK6, TACSTD2, CD123, CD33, CD138, CS1, CD133, CD52, TNFRSF13C, TNFRSF13B, CXCR4, PD-L1, LY9, CD200, FCGR2B, CD21, CD23, or CD40L.

In some embodiments, TAA 1 and TAA2 are both mesothelin. In some embodiments, TAA 1 and TAA2 are both TSHR. In some embodiments, TAA 1 and TAA2 are both CD171. In some embodiments, TAA 1 and TAA2 are both CS-1. In some embodiments, TAA 1 and TAA2 are both GD3. In some embodiments, TAA 1 and TAA2 are both Tn Ag. In some embodiments, TAA 1 and TAA2 are both CD44v6. In some embodiments, TAA 1 and TAA2 are both B7H3. In some embodiments, TAA 1 and TAA2 are both KIT. In some embodiments, TAA 1 and TAA2 are both IL-13Ra2. In some embodiments, TAA 1 and TAA2 are both IL-11Ra. In some embodiments, TAA 1 and TAA2 are both PSCA. In some embodiments, TAA 1 and TAA2 are both PRSS21. In some embodiments, TAA 1 and TAA2 are both VEGFR2. In some embodiments, TAA 1 and TAA2 are both LewisY. In some embodiments, TAA 1 and TAA2 are both PDGFR-beta. In some embodiments, TAA 1 and TAA2 are both SSEA-4. In some embodiments, TAA 1 and TAA2 are both MUC1. In some embodiments, TAA 1 and TAA2 are both EGFR. In some embodiments, TAA 1 and TAA2 are both NCAM. In some embodiments, TAA 1 and TAA2 are both CAIX. In some embodiments, TAA 1 and TAA2 are both LMP2. In some embodiments, TAA 1 and TAA2 are both EphA2. In some embodiments, TAA 1 and TAA2 are both fucosyl GM1. In some embodiments, TAA 1 and TAA2 are both sLe. In some embodiments, TAA 1 and TAA2 are both GM3. In some embodiments, TAA 1 and TAA2 are both TGS5. In some embodiments, TAA 1 and TAA2 are both HMWMAA. In some embodiments, TAA 1 and TAA2 are both o-acetyl-GD2. In some embodiments, TAA 1 and TAA2 are both GD2. In some embodiments, TAA 1 and TAA2 are both folate receptor alpha. In some embodiments, TAA 1 and TAA2 are both folate receptor beta. In some embodiments, TAA 1 and TAA2 are both TEM1/CD248. In some embodiments, TAA 1 and TAA2 are both TEM7R. In some embodiments, TAA 1 and TAA2 are both CLDN6. In some embodiments, TAA 1 and TAA2 are both GPRC5D. In some embodiments, TAA 1 and TAA2 are both CXORF61. In some embodiments, TAA 1 and TAA2 are both CD97. In some embodiments, TAA 1 and TAA2 are both CD179a. In some embodiments, TAA 1 and TAA2 are both ALK. In some embodiments, TAA 1 and TAA2 are both polysialic acid. In some embodiments, TAA 1 and TAA2 are both PLAC1. In some embodiments, TAA 1 and TAA2 are both GloboH. In some embodiments, TAA 1 and TAA2 are both NY-BR-1. In some embodiments, TAA 1 and TAA2 are both UPK2. In some embodiments, TAA 1 and TAA2 are both HAVCR1. In some embodiments, TAA 1 and TAA2 are both ADRB3. In some embodiments, TAA 1 and TAA2 are both PANX3. In some embodiments, TAA 1 and TAA2 are both GPR20. In some embodiments, TAA 1 and TAA2 are both LY6K. In some embodiments, TAA 1 and TAA2 are both OR51E2. In some embodiments, TAA 1 and TAA2 are both TAARP. In some embodiments, TAA 1 and TAA2 are both WT1. In some embodiments, TAA 1 and TAA2 are both ETV6-AML. In some embodiments, TAA 1 and TAA2 are both sperm protein 17. In some embodiments, TAA 1 and TAA2 are both XAGE1. In some embodiments, TAA 1 and TAA2 are both Tie 2. In some embodiments, TAA 1 and TAA2 are both MAD-CT-1. In some embodiments, TAA 1 and TAA2 are both MAD-CT-2. In some embodiments, TAA 1 and TAA2 are both Fos-related antigen 1. In some embodiments, TAA 1 and TAA2 are both p53 mutant. In some embodiments, TAA 1 and TAA2 are both hTERT. In some embodiments, TAA 1 and TAA2 are both sarcoma translocation breakpoints. In some embodiments, TAA 1 and TAA2 are both ML-IAP. In some embodiments, TAA 1 and TAA2 are both ERG (TMPRSS2 ETS fusion gene). In some embodiments, TAA 1 and TAA2 are both NA17. In some embodiments, TAA 1 and TAA2 are both PAX3. In some embodiments, TAA 1 and TAA2 are both Androgen receptor. In some embodiments, TAA 1 and TAA2 are both Cyclin B1. In some embodiments, TAA 1 and TAA2 are both MYCN. In some embodiments, TAA 1 and TAA2 are both RhoC. In some embodiments, TAA 1 and TAA2 are both CYP1B1. In some embodiments, TAA 1 and TAA2 are both BORIS. In some embodiments, TAA 1 and TAA2 are both SART3. In some embodiments, TAA 1 and TAA2 are both PAX5. In some embodiments, TAA 1 and TAA2 are both OY-TES1. In some embodiments, TAA 1 and TAA2 are both LCK. In some embodiments, TAA 1 and TAA2 are both AKAP-4. In some embodiments, TAA 1 and TAA2 are both SSX2. In some embodiments, TAA 1 and TAA2 are both LAIR1. In some embodiments, TAA 1 and TAA2 are both FCAR. In some embodiments, TAA 1 and TAA2 are both LILRA2. In some embodiments, TAA 1 and TAA2 are both CD300LF. In some embodiments, TAA 1 and TAA2 are both CLEC12A. In some embodiments, TAA 1 and TAA2 are both BST2. In some embodiments, TAA 1 and TAA2 are both EMR2. In some embodiments, TAA 1 and TAA2 are both LY75. In some embodiments, TAA 1 and TAA2 are both GPC3. In some embodiments, TAA 1 and TAA2 are both FCRL5. In some embodiments, TAA 1 and TAA2 are both IGLL1. In some embodiments, TAA 1 and TAA2 are both CD30. In some embodiments, TAA 1 and TAA2 are both ERBB2. In some embodiments, TAA 1 and TAA2 are both ROR1. In some embodiments, TAA 1 and TAA2 are both TAAG72. In some embodiments, TAA 1 and TAA2 are both GD2. In some embodiments, TAA 1 and TAA2 are both gp100Tn. In some embodiments, TAA 1 and TAA2 are both FAP. In some embodiments, TAA 1 and TAA2 are both tyrosinase. In some embodiments, TAA 1 and TAA2 are both EPCAM. In some embodiments, TAA 1 and TAA2 are both CEA. In some embodiments, TAA 1 and TAA2 are both Igf-I receptor. In some embodiments, TAA 1 and TAA2 are both EphB2. In some embodiments, TAA 1 and TAA2 are both Cadherin17. In some embodiments, TAA 1 and TAA2 are both CD32b. In some embodiments, TAA 1 and TAA2 are both EGFRvIII. In some embodiments, TAA 1 and TAA2 are both GPNMB. In some embodiments, TAA 1 and TAA2 are both GPR64. In some embodiments, TAA 1 and TAA2 are both HER3. In some embodiments, TAA 1 and TAA2 are both LRP6. In some embodiments, TAA 1 and TAA2 are both LYPD8. In some embodiments, TAA 1 and TAA2 are both NKG2D. In some embodiments, TAA 1 and TAA2 are both SLC34A2. In some embodiments, TAA 1 and TAA2 are both SLC39A6. In some embodiments, TAA 1 and TAA2 are both SLITRK6. In some embodiments, TAA 1 and TAA2 are both TACSTD2.

In some embodiments, a TAA (e.g., TAA 1 and/or TAA 2) targeted by a MBM of the disclosure is CD19, CD20, CD22, CD123, BCMA, CD33, CLL-1, CD138, CS1, CD38, CD133, FLT3, CD52, ENPP1, TNFRSF13C, TNFRSF13B, CXCR4, PD-L1, LY9, CD200, FCGR2B, CD21, CD23, CD24, CD40L, CD72, CD74, CD79a, CD79b, CD93, or CD99.

In some combinations of MBMs, TAA 1 and TAA 2 are selected from CD19, CD20, CD22, CD123, BCMA, CD33, CLL-1, CD138, CS1, CD38, CD133, FLT3, CD52, ENPP1, TNFRSF13C, TNFRSF13B, CXCR4, PD-L1, LY9, CD200, FCGR2B, CD21, CD23, CD24, CD40L, CD72, CD79a, and CD79b. In some embodiments, TAA 1 is CD19 and TAA 2 is CD20 (or vice versa). In some embodiments, TAA 1 is CD19 and TAA 2 is CD22 (or vice versa). In some embodiments, TAA 1 is CD19 and TAA 2 is CD123 (or vice versa). In some embodiments, TAA 1 is CD19 and TAA 2 is BCMA (or vice versa). In some embodiments, TAA 1 is CD19 and TAA 2 is CD33 (or vice versa). In some embodiments, TAA 1 is CD19 and TAA 2 is CLL1 (or vice versa). In some embodiments, TAA 1 is CD19 and TAA 2 is CD138 (or vice versa). In some embodiments, TAA 1 is CD19 and TAA 2 is CS1 (or vice versa). In some embodiments, TAA 1 is CD19 and TAA 2 is CD38 (or vice versa). In some embodiments, TAA 1 is CD19 and TAA 2 is CD133 (or vice versa). In some embodiments, TAA 1 is CD19 and TAA 2 is FLT3 (or vice versa). In some embodiments, TAA 1 is CD19 and TAA 2 is CD52 (or vice versa). In some embodiments, TAA 1 is CD19 and TAA 2 is TNFRSF13C (or vice versa). In some embodiments, TAA 1 is CD19 and TAA 2 is TNFRSF13B (or vice versa). In some embodiments, TAA 1 is CD19 and TAA 2 is CXCR4 (or vice versa). In some embodiments, TAA 1 is CD19 and TAA 2 is PD-L1 (or vice versa). In some embodiments, TAA 1 is CD19 and TAA 2 is LY9 (or vice versa). In some embodiments, TAA 1 is CD19 and TAA 2 is CD200 (or vice versa). In some embodiments, TAA 1 is CD19 and TAA 2 is FCGR2B (or vice versa). In some embodiments, TAA 1 is CD19 and TAA 2 is CD21 (or vice versa). In some embodiments, TAA 1 is CD19 and TAA 2 is CD23 (or vice versa). In some embodiments, TAA 1 is CD19 and TAA 2 is CD24 (or vice versa). In some embodiments, TAA 1 is CD19 and TAA 2 is CD40L (or vice versa). In some embodiments, TAA 1 is CD19 and TAA 2 is CD72 (or vice versa). In some embodiments, TAA 1 is CD19 and TAA 2 is CD79a (or vice versa). In some embodiments, TAA 1 is CD19 and TAA 2 is CD79b (or vice versa). In some embodiments, TAA 1 is CD20 and TAA 2 is CD22 (or vice versa). In some embodiments, TAA 1 is CD20 and TAA 2 is CD123 (or vice versa). In some embodiments, TAA 1 is CD20 and TAA 2 is BCMA (or vice versa). In some embodiments, TAA 1 is CD20 and TAA 2 is CD33 (or vice versa). In some embodiments, TAA 1 is CD20 and TAA 2 is CLL1 (or vice versa). In some embodiments, TAA 1 is CD20 and TAA 2 is CD138 (or vice versa). In some embodiments, TAA 1 is CD20 and TAA 2 is CS1 (or vice versa). In some embodiments, TAA 1 is CD20 and TAA 2 is CD38 (or vice versa). In some embodiments, TAA 1 is CD20 and TAA 2 is CD133 (or vice versa). In some embodiments, TAA 1 is CD20 and TAA 2 is FLT3 (or vice versa). In some embodiments, TAA 1 is CD20 and TAA 2 is CD52 (or vice versa). In some embodiments, TAA 1 is CD20 and TAA 2 is TNFRSF13C (or vice versa). In some embodiments, TAA 1 is CD20 and TAA 2 is TNFRSF13B (or vice versa). In some embodiments, TAA 1 is CD20 and TAA 2 is CXCR4 (or vice versa). In some embodiments, TAA 1 is CD20 and TAA 2 is PD-L1 (or vice versa). In some embodiments, TAA 1 is CD20 and TAA 2 is LY9 (or vice versa). In some embodiments, TAA 1 is CD20 and TAA 2 is CD200 (or vice versa). In some embodiments, TAA 1 is CD20 and TAA 2 is FCGR2B (or vice versa). In some embodiments, TAA 1 is CD20 and TAA 2 is CD21 (or vice versa). In some embodiments, TAA 1 is CD20 and TAA 2 is CD23 (or vice versa). In some embodiments, TAA 1 is CD20 and TAA 2 is CD24 (or vice versa). In some embodiments, TAA 1 is CD20 and TAA 2 is CD40L (or vice versa). In some embodiments, TAA 1 is CD20 and TAA 2 is CD72 (or vice versa). In some embodiments, TAA 1 is CD20 and TAA 2 is CD79a (or vice versa). In some embodiments, TAA 1 is CD20 and TAA 2 is CD79b (or vice versa). In some embodiments, TAA 1 is CD22 and TAA 2 is CD123 (or vice versa). In some embodiments, TAA 1 is CD22 and TAA 2 is BCMA (or vice versa). In some embodiments, TAA 1 is CD22 and TAA 2 is CD33 (or vice versa). In some embodiments, TAA 1 is CD22 and TAA 2 is CLL1 (or vice versa). In some embodiments, TAA 1 is CD22 and TAA 2 is CD138 (or vice versa). In some embodiments, TAA 1 is CD22 and TAA 2 is CS1 (or vice versa). In some embodiments, TAA 1 is CD22 and TAA 2 is CD38 (or vice versa). In some embodiments, TAA 1 is CD22 and TAA 2 is CD133 (or vice versa). In some embodiments, TAA 1 is CD22 and TAA 2 is FLT3 (or vice versa). In some embodiments, TAA 1 is CD22 and TAA 2 is CD52 (or vice versa). In some embodiments, TAA 1 is CD22 and TAA 2 is TNFRSF13C (or vice versa). In some embodiments, TAA 1 is CD22 and TAA 2 is TNFRSF13B (or vice versa). In some embodiments, TAA 1 is CD22 and TAA 2 is CXCR4 (or vice versa). In some embodiments, TAA 1 is CD22 and TAA 2 is PD-L1 (or vice versa). In some embodiments, TAA 1 is CD22 and TAA 2 is LY9 (or vice versa). In some embodiments, TAA 1 is CD22 and TAA 2 is CD200 (or vice versa). In some embodiments, TAA 1 is CD22 and TAA 2 is FCGR2B (or vice versa). In some embodiments, TAA 1 is CD22 and TAA 2 is CD21 (or vice versa). In some embodiments, TAA 1 is CD22 and TAA 2 is CD23 (or vice versa). In some embodiments, TAA 1 is CD22 and TAA 2 is CD24 (or vice versa). In some embodiments, TAA 1 is CD22 and TAA 2 is CD40L (or vice versa). In some embodiments, TAA 1 is CD22 and TAA 2 is CD72 (or vice versa). In some embodiments, TAA 1 is CD22 and TAA 2 is CD79a (or vice versa). In some embodiments, TAA 1 is CD22 and TAA 2 is CD79b (or vice versa). In some embodiments, TAA 1 is CD123 and TAA 2 is BCMA (or vice versa). In some embodiments, TAA 1 is CD123 and TAA 2 is CD33 (or vice versa). In some embodiments, TAA 1 is CD123 and TAA 2 is CLL1 (or vice versa). In some embodiments, TAA 1 is CD123 and TAA 2 is CD138 (or vice versa). In some embodiments, TAA 1 is CD123 and TAA 2 is CS1 (or vice versa). In some embodiments, TAA 1 is CD123 and TAA 2 is CD38 (or vice versa). In some embodiments, TAA 1 is CD123 and TAA 2 is CD133 (or vice versa). In some embodiments, TAA 1 is CD123 and TAA 2 is FLT3 (or vice versa). In some embodiments, TAA 1 is CD123 and TAA 2 is CD52 (or vice versa). In some embodiments, TAA 1 is CD123 and TAA 2 is TNFRSF13C (or vice versa). In some embodiments, TAA 1 is CD123 and TAA 2 is TNFRSF13B (or vice versa). In some embodiments, TAA 1 is CD123 and TAA 2 is CXCR4 (or vice versa). In some embodiments, TAA 1 is CD123 and TAA 2 is PD-L1 (or vice versa). In some embodiments, TAA 1 is CD123 and TAA 2 is LY9 (or vice versa). In some embodiments, TAA 1 is CD123 and TAA 2 is CD200 (or vice versa). In some embodiments, TAA 1 is CD123 and TAA 2 is FCGR2B (or vice versa). In some embodiments, TAA 1 is CD123 and TAA 2 is CD21 (or vice versa). In some embodiments, TAA 1 is CD123 and TAA 2 is CD23 (or vice versa). In some embodiments, TAA 1 is CD123 and TAA 2 is CD24 (or vice versa). In some embodiments, TAA 1 is CD123 and TAA 2 is CD40L (or vice versa). In some embodiments, TAA 1 is CD123 and TAA 2 is CD72 (or vice versa). In some embodiments, TAA 1 is CD123 and TAA 2 is CD79a (or vice versa). In some embodiments, TAA 1 is CD123 and TAA 2 is CD79b (or vice versa). In some embodiments, TAA 1 is BCMA and TAA 2 is CD33 (or vice versa). In some embodiments, TAA 1 is BCMA and TAA 2 is CLL1 (or vice versa). In some embodiments, TAA 1 is BCMA and TAA 2 is CD138 (or vice versa). In some embodiments, TAA 1 is BCMA and TAA 2 is CS1 (or vice versa). In some embodiments, TAA 1 is BCMA and TAA 2 is CD38 (or vice versa). In some embodiments, TAA 1 is BCMA and TAA 2 is CD133 (or vice versa). In some embodiments, TAA 1 is BCMA and TAA 2 is FLT3 (or vice versa). In some embodiments, TAA 1 is BCMA and TAA 2 is CD52 (or vice versa). In some embodiments, TAA 1 is BCMA and TAA 2 is TNFRSF13C (or vice versa). In some embodiments, TAA 1 is BCMA and TAA 2 is TNFRSF13B (or vice versa). In some embodiments, TAA 1 is BCMA and TAA 2 is CXCR4 (or vice versa). In some embodiments, TAA 1 is BCMA and TAA 2 is PD-L1 (or vice versa). In some embodiments, TAA 1 is BCMA and TAA 2 is LY9 (or vice versa). In some embodiments, TAA 1 is BCMA and TAA 2 is CD200 (or vice versa). In some embodiments, TAA 1 is BCMA and TAA 2 is FCGR2B (or vice versa). In some embodiments, TAA 1 is BCMA and TAA 2 is CD21 (or vice versa). In some embodiments, TAA 1 is BCMA and TAA 2 is CD23 (or vice versa). In some embodiments, TAA 1 is BCMA and TAA 2 is CD24 (or vice versa). In some embodiments, TAA 1 is BCMA and TAA 2 is CD40L (or vice versa). In some embodiments, TAA 1 is BCMA and TAA 2 is CD72 (or vice versa). In some embodiments, TAA 1 is BCMA and TAA 2 is CD79a (or vice versa). In some embodiments, TAA 1 is BCMA and TAA 2 is CD79b (or vice versa). In some embodiments, TAA 1 is CD33 and TAA 2 is CLL1 (or vice versa). In some embodiments, TAA 1 is CD33 and TAA 2 is CD138 (or vice versa). In some embodiments, TAA 1 is CD33 and TAA 2 is CS1 (or vice versa). In some embodiments, TAA 1 is CD33 and TAA 2 is CD38 (or vice versa). In some embodiments, TAA 1 is CD33 and TAA 2 is CD133 (or vice versa). In some embodiments, TAA 1 is CD33 and TAA 2 is FLT3 (or vice versa). In some embodiments, TAA 1 is CD33 and TAA 2 is CD52 (or vice versa). In some embodiments, TAA 1 is CD33 and TAA 2 is TNFRSF13C (or vice versa). In some embodiments, TAA 1 is CD33 and TAA 2 is TNFRSF13B (or vice versa). In some embodiments, TAA 1 is CD33 and TAA 2 is CXCR4 (or vice versa). In some embodiments, TAA 1 is CD33 and TAA 2 is PD-L1 (or vice versa). In some embodiments, TAA 1 is CD33 and TAA 2 is LY9 (or vice versa). In some embodiments, TAA 1 is CD33 and TAA 2 is CD200 (or vice versa). In some embodiments, TAA 1 is CD33 and TAA 2 is FCGR2B (or vice versa). In some embodiments, TAA 1 is CD33 and TAA 2 is CD21 (or vice versa). In some embodiments, TAA 1 is CD33 and TAA 2 is CD23 (or vice versa). In some embodiments, TAA 1 is CD33 and TAA 2 is CD24 (or vice versa). In some embodiments, TAA 1 is CD33 and TAA 2 is CD40L (or vice versa). In some embodiments, TAA 1 is CD33 and TAA 2 is CD72 (or vice versa). In some embodiments, TAA 1 is CD33 and TAA 2 is CD79a (or vice versa). In some embodiments, TAA 1 is CD33 and TAA 2 is CD79b (or vice versa). In some embodiments, TAA 1 is CLL1 and TAA 2 is CD138 (or vice versa). In some embodiments, TAA 1 is CLL1 and TAA 2 is CS1 (or vice versa). In some embodiments, TAA 1 is CLL1 and TAA 2 is CD38 (or vice versa). In some embodiments, TAA 1 is CLL1 and TAA 2 is CD133 (or vice versa). In some embodiments, TAA 1 is CLL1 and TAA 2 is FLT3 (or vice versa). In some embodiments, TAA 1 is CLL1 and TAA 2 is CD52 (or vice versa). In some embodiments, TAA 1 is CLL1 and TAA 2 is TNFRSF13C (or vice versa). In some embodiments, TAA 1 is CLL1 and TAA 2 is TNFRSF13B (or vice versa). In some embodiments, TAA 1 is CLL1 and TAA 2 is CXCR4 (or vice versa). In some embodiments, TAA 1 is CLL1 and TAA 2 is PD-L1 (or vice versa). In some embodiments, TAA 1 is CLL1 and TAA 2 is LY9 (or vice versa). In some embodiments, TAA 1 is CLL1 and TAA 2 is CD200 (or vice versa). In some embodiments, TAA 1 is CLL1 and TAA 2 is FCGR2B (or vice versa). In some embodiments, TAA 1 is CLL1 and TAA 2 is CD21 (or vice versa). In some embodiments, TAA 1 is CLL1 and TAA 2 is CD23 (or vice versa). In some embodiments, TAA 1 is CLL1 and TAA 2 is CD24 (or vice versa). In some embodiments, TAA 1 is CLL1 and TAA 2 is CD40L (or vice versa). In some embodiments, TAA 1 is CLL1 and TAA 2 is CD72 (or vice versa). In some embodiments, TAA 1 is CLL1 and TAA 2 is CD79a (or vice versa). In some embodiments, TAA 1 is CLL1 and TAA 2 is CD79b (or vice versa). In some embodiments, TAA 1 is CD138 and TAA 2 is CS1 (or vice versa). In some embodiments, TAA 1 is CD138 and TAA 2 is CD38 (or vice versa). In some embodiments, TAA 1 is CD138 and TAA 2 is CD133 (or vice versa). In some embodiments, TAA 1 is CD138 and TAA 2 is FLT3 (or vice versa). In some embodiments, TAA 1 is CD138 and TAA 2 is CD52 (or vice versa). In some embodiments, TAA 1 is CD138 and TAA 2 is TNFRSF13C (or vice versa). In some embodiments, TAA 1 is CD138 and TAA 2 is TNFRSF13B (or vice versa). In some embodiments, TAA 1 is CD138 and TAA 2 is CXCR4 (or vice versa). In some embodiments, TAA 1 is CD138 and TAA 2 is PD-L1 (or vice versa). In some embodiments, TAA 1 is CD138 and TAA 2 is LY9 (or vice versa). In some embodiments, TAA 1 is CD138 and TAA 2 is CD200 (or vice versa). In some embodiments, TAA 1 is CD138 and TAA 2 is FCGR2B (or vice versa). In some embodiments, TAA 1 is CD138 and TAA 2 is CD21 (or vice versa). In some embodiments, TAA 1 is CD138 and TAA 2 is CD23 (or vice versa). In some embodiments, TAA 1 is CD138 and TAA 2 is CD24 (or vice versa). In some embodiments, TAA 1 is CD138 and TAA 2 is CD40L (or vice versa). In some embodiments, TAA 1 is CD138 and TAA 2 is CD72 (or vice versa). In some embodiments, TAA 1 is CD138 and TAA 2 is CD79a (or vice versa). In some embodiments, TAA 1 is CD138 and TAA 2 is CD79b (or vice versa). In some embodiments, TAA 1 is CS1 and TAA 2 is CD38 (or vice versa). In some embodiments, TAA 1 is CS1 and TAA 2 is CD133 (or vice versa). In some embodiments, TAA 1 is CS1 and TAA 2 is FLT3 (or vice versa). In some embodiments, TAA 1 is CS1 and TAA 2 is CD52 (or vice versa). In some embodiments, TAA 1 is CS1 and TAA 2 is TNFRSF13C (or vice versa). In some embodiments, TAA 1 is CS1 and TAA 2 is TNFRSF13B (or vice versa). In some embodiments, TAA 1 is CS1 and TAA 2 is CXCR4 (or vice versa). In some embodiments, TAA 1 is CS1 and TAA 2 is PD-L1 (or vice versa). In some embodiments, TAA 1 is CS1 and TAA 2 is LY9 (or vice versa). In some embodiments, TAA 1 is CS1 and TAA 2 is CD200 (or vice versa). In some embodiments, TAA 1 is CS1 and TAA 2 is FCGR2B (or vice versa). In some embodiments, TAA 1 is CS1 and TAA 2 is CD21 (or vice versa). In some embodiments, TAA 1 is CS1 and TAA 2 is CD23 (or vice versa). In some embodiments, TAA 1 is CS1 and TAA 2 is CD24 (or vice versa). In some embodiments, TAA 1 is CS1 and TAA 2 is CD40L (or vice versa). In some embodiments, TAA 1 is CS1 and TAA 2 is CD72 (or vice versa). In some embodiments, TAA 1 is CS1 and TAA 2 is CD79a (or vice versa). In some embodiments, TAA 1 is CS1 and TAA 2 is CD79b (or vice versa). In some embodiments, TAA 1 is CD38 and TAA 2 is CD133 (or vice versa). In some embodiments, TAA 1 is CD38 and TAA 2 is FLT3 (or vice versa). In some embodiments, TAA 1 is CD38 and TAA 2 is CD52 (or vice versa). In some embodiments, TAA 1 is CD38 and TAA 2 is TNFRSF13C (or vice versa). In some embodiments, TAA 1 is CD38 and TAA 2 is TNFRSF13B (or vice versa). In some embodiments, TAA 1 is CD38 and TAA 2 is CXCR4 (or vice versa). In some embodiments, TAA 1 is CD38 and TAA 2 is PD-L1 (or vice versa). In some embodiments, TAA 1 is CD38 and TAA 2 is LY9 (or vice versa). In some embodiments, TAA 1 is CD38 and TAA 2 is CD200 (or vice versa). In some embodiments, TAA 1 is CD38 and TAA 2 is FCGR2B (or vice versa). In some embodiments, TAA 1 is CD38 and TAA 2 is CD21 (or vice versa). In some embodiments, TAA 1 is CD38 and TAA 2 is CD23 (or vice versa). In some embodiments, TAA 1 is CD38 and TAA 2 is CD24 (or vice versa). In some embodiments, TAA 1 is CD38 and TAA 2 is CD40L (or vice versa). In some embodiments, TAA 1 is CD38 and TAA 2 is CD72 (or vice versa). In some embodiments, TAA 1 is CD38 and TAA 2 is CD79a (or vice versa). In some embodiments, TAA 1 is CD38 and TAA 2 is CD79b (or vice versa). In some embodiments, TAA 1 is CD133 and TAA 2 is FLT3 (or vice versa). In some embodiments, TAA 1 is CD133 and TAA 2 is CD52 (or vice versa). In some embodiments, TAA 1 is CD133 and TAA 2 is TNFRSF13C (or vice versa). In some embodiments, TAA 1 is CD133 and TAA 2 is TNFRSF13B (or vice versa). In some embodiments, TAA 1 is CD133 and TAA 2 is CXCR4 (or vice versa). In some embodiments, TAA 1 is CD133 and TAA 2 is PD-L1 (or vice versa). In some embodiments, TAA 1 is CD133 and TAA 2 is LY9 (or vice versa). In some embodiments, TAA 1 is CD133 and TAA 2 is CD200 (or vice versa). In some embodiments, TAA 1 is CD133 and TAA 2 is FCGR2B (or vice versa). In some embodiments, TAA 1 is CD133 and TAA 2 is CD21 (or vice versa). In some embodiments, TAA 1 is CD133 and TAA 2 is CD23 (or vice versa). In some embodiments, TAA 1 is CD133 and TAA 2 is CD24 (or vice versa). In some embodiments, TAA 1 is CD133 and TAA 2 is CD40L (or vice versa). In some embodiments, TAA 1 is CD133 and TAA 2 is CD72 (or vice versa). In some embodiments, TAA 1 is CD133 and TAA 2 is CD79a (or vice versa). In some embodiments, TAA 1 is CD133 and TAA 2 is CD79b (or vice versa). In some embodiments, TAA 1 is FLT3 and TAA 2 is CD52 (or vice versa). In some embodiments, TAA 1 is FLT3 and TAA 2 is TNFRSF13C (or vice versa). In some embodiments, TAA 1 is FLT3 and TAA 2 is TNFRSF13B (or vice versa). In some embodiments, TAA 1 is FLT3 and TAA 2 is CXCR4 (or vice versa). In some embodiments, TAA 1 is FLT3 and TAA 2 is PD-L1 (or vice versa). In some embodiments, TAA 1 is FLT3 and TAA 2 is LY9 (or vice versa). In some embodiments, TAA 1 is FLT3 and TAA 2 is CD200 (or vice versa). In some embodiments, TAA 1 is FLT3 and TAA 2 is FCGR2B (or vice versa). In some embodiments, TAA 1 is FLT3 and TAA 2 is CD21 (or vice versa). In some embodiments, TAA 1 is FLT3 and TAA 2 is CD23 (or vice versa). In some embodiments, TAA 1 is FLT3 and TAA 2 is CD24 (or vice versa). In some embodiments, TAA 1 is FLT3 and TAA 2 is CD40L (or vice versa). In some embodiments, TAA 1 is FLT3 and TAA 2 is CD72 (or vice versa). In some embodiments, TAA 1 is FLT3 and TAA 2 is CD79a (or vice versa). In some embodiments, TAA 1 is FLT3 and TAA 2 is CD79b (or vice versa). In some embodiments, TAA 1 is CD52 and TAA 2 is TNFRSF13C (or vice versa). In some embodiments, TAA 1 is CD52 and TAA 2 is TNFRSF13B (or vice versa). In some embodiments, TAA 1 is CD52 and TAA 2 is CXCR4 (or vice versa). In some embodiments, TAA 1 is CD52 and TAA 2 is PD-L1 (or vice versa). In some embodiments, TAA 1 is CD52 and TAA 2 is LY9 (or vice versa). In some embodiments, TAA 1 is CD52 and TAA 2 is CD200 (or vice versa). In some embodiments, TAA 1 is CD52 and TAA 2 is FCGR2B (or vice versa). In some embodiments, TAA 1 is CD52 and TAA 2 is CD21 (or vice versa). In some embodiments, TAA 1 is CD52 and TAA 2 is CD23 (or vice versa). In some embodiments, TAA 1 is CD52 and TAA 2 is CD24 (or vice versa). In some embodiments, TAA 1 is CD52 and TAA 2 is CD40L (or vice versa). In some embodiments, TAA 1 is CD52 and TAA 2 is CD72 (or vice versa). In some embodiments, TAA 1 is CD52 and TAA 2 is CD79a (or vice versa). In some embodiments, TAA 1 is CD52 and TAA 2 is CD79b (or vice versa). In some embodiments, TAA 1 is TNFRSF13C and TAA 2 is TNFRSF13B (or vice versa). In some embodiments, TAA 1 is TNFRSF13C and TAA 2 is CXCR4 (or vice versa). In some embodiments, TAA 1 is TNFRSF13C and TAA 2 is PD-L1 (or vice versa). In some embodiments, TAA 1 is TNFRSF13C and TAA 2 is LY9 (or vice versa). In some embodiments, TAA 1 is TNFRSF13C and TAA 2 is CD200 (or vice versa). In some embodiments, TAA 1 is TNFRSF13C and TAA 2 is FCGR2B (or vice versa). In some embodiments, TAA 1 is TNFRSF13C and TAA 2 is CD21 (or vice versa). In some embodiments, TAA 1 is TNFRSF13C and TAA 2 is CD23 (or vice versa). In some embodiments, TAA 1 is TNFRSF13C and TAA 2 is CD24 (or vice versa). In some embodiments, TAA 1 is TNFRSF13C and TAA 2 is CD40L (or vice versa). In some embodiments, TAA 1 is TNFRSF13C and TAA 2 is CD72 (or vice versa). In some embodiments, TAA 1 is TNFRSF13C and TAA 2 is CD79a (or vice versa). In some embodiments, TAA 1 is TNFRSF13C and TAA 2 is CD79b (or vice versa). In some embodiments, TAA 1 is TNFRSF13B and TAA 2 is CXCR4 (or vice versa). In some embodiments, TAA 1 is TNFRSF13B and TAA 2 is PD-L1 (or vice versa). In some embodiments, TAA 1 is TNFRSF13B and TAA 2 is LY9 (or vice versa). In some embodiments, TAA 1 is TNFRSF13B and TAA 2 is CD200 (or vice versa). In some embodiments, TAA 1 is TNFRSF13B and TAA 2 is FCGR2B (or vice versa). In some embodiments, TAA 1 is TNFRSF13B and TAA 2 is CD21 (or vice versa). In some embodiments, TAA 1 is TNFRSF13B and TAA 2 is CD23 (or vice versa). In some embodiments, TAA 1 is TNFRSF13B and TAA 2 is CD24 (or vice versa). In some embodiments, TAA 1 is TNFRSF13B and TAA 2 is CD40L (or vice versa). In some embodiments, TAA 1 is TNFRSF13B and TAA 2 is CD72 (or vice versa). In some embodiments, TAA 1 is TNFRSF13B and TAA 2 is CD79a (or vice versa). In some embodiments, TAA 1 is TNFRSF13B and TAA 2 is CD79b (or vice versa). In some embodiments, TAA 1 is CXCR4 and TAA 2 is PD-L1 (or vice versa). In some embodiments, TAA 1 is CXCR4 and TAA 2 is LY9 (or vice versa). In some embodiments, TAA 1 is CXCR4 and TAA 2 is CD200 (or vice versa). In some embodiments, TAA 1 is CXCR4 and TAA 2 is FCGR2B (or vice versa). In some embodiments, TAA 1 is CXCR4 and TAA 2 is CD21 (or vice versa). In some embodiments, TAA 1 is CXCR4 and TAA 2 is CD23 (or vice versa). In some embodiments, TAA 1 is CXCR4 and TAA 2 is CD24 (or vice versa). In some embodiments, TAA 1 is CXCR4 and TAA 2 is CD40L (or vice versa). In some embodiments, TAA 1 is CXCR4 and TAA 2 is CD72 (or vice versa). In some embodiments, TAA 1 is CXCR4 and TAA 2 is CD79a (or vice versa). In some embodiments, TAA 1 is CXCR4 and TAA 2 is CD79b (or vice versa). In some embodiments, TAA 1 is PD-L1 and TAA 2 is LY9 (or vice versa). In some embodiments, TAA 1 is PD-L1 and TAA 2 is CD200 (or vice versa). In some embodiments, TAA 1 is PD-L1 and TAA 2 is FCGR2B (or vice versa). In some embodiments, TAA 1 is PD-L1 and TAA 2 is CD21 (or vice versa). In some embodiments, TAA 1 is PD-L1 and TAA 2 is CD23 (or vice versa). In some embodiments, TAA 1 is PD-L1 and TAA 2 is CD24 (or vice versa). In some embodiments, TAA 1 is PD-L1 and TAA 2 is CD40L (or vice versa). In some embodiments, TAA 1 is PD-L1 and TAA 2 is CD72 (or vice versa). In some embodiments, TAA 1 is PD-L1 and TAA 2 is CD79a (or vice versa). In some embodiments, TAA 1 is PD-L1 and TAA 2 is CD79b (or vice versa). In some embodiments, TAA 1 is LY9 and TAA 2 is CD200 (or vice versa). In some embodiments, TAA 1 is LY9 and TAA 2 is FCGR2B (or vice versa). In some embodiments, TAA 1 is LY9 and TAA 2 is CD21 (or vice versa). In some embodiments, TAA 1 is LY9 and TAA 2 is CD23 (or vice versa). In some embodiments, TAA 1 is LY9 and TAA 2 is CD24 (or vice versa). In some embodiments, TAA 1 is LY9 and TAA 2 is CD40L (or vice versa). In some embodiments, TAA 1 is LY9 and TAA 2 is CD72 (or vice versa). In some embodiments, TAA 1 is LY9 and TAA 2 is CD79a (or vice versa). In some embodiments, TAA 1 is LY9 and TAA 2 is CD79b (or vice versa). In some embodiments, TAA 1 is CD200 and TAA 2 is FCGR2B (or vice versa). In some embodiments, TAA 1 is CD200 and TAA 2 is CD21 (or vice versa). In some embodiments, TAA 1 is CD200 and TAA 2 is CD23 (or vice versa). In some embodiments, TAA 1 is CD200 and TAA 2 is CD24 (or vice versa). In some embodiments, TAA 1 is CD200 and TAA 2 is CD40L (or vice versa). In some embodiments, TAA 1 is CD200 and TAA 2 is CD72 (or vice versa). In some embodiments, TAA 1 is CD200 and TAA 2 is CD79a (or vice versa). In some embodiments, TAA 1 is CD200 and TAA 2 is CD79b (or vice versa). In some embodiments, TAA 1 is CD21 and TAA 2 is CD23 (or vice versa). In some embodiments, TAA 1 is CD21 and TAA 2 is CD24 (or vice versa). In some embodiments, TAA 1 is CD21 and TAA 2 is CD40L (or vice versa). In some embodiments, TAA 1 is CD21 and TAA 2 is CD72 (or vice versa). In some embodiments, TAA 1 is CD21 and TAA 2 is CD79a (or vice versa). In some embodiments, TAA 1 is CD21 and TAA 2 is CD79b (or vice versa). In some embodiments, TAA 1 is CD23 and TAA 2 is CD24 (or vice versa). In some embodiments, TAA 1 is CD23 and TAA 2 is CD40L (or vice versa). In some embodiments, TAA 1 is CD23 and TAA 2 is CD72 (or vice versa). In some embodiments, TAA 1 is CD23 and TAA 2 is CD79a (or vice versa). In some embodiments, TAA 1 is CD23 and TAA 2 is CD79b (or vice versa). In some embodiments, TAA 1 is CD24 and TAA 2 is CD40L (or vice versa). In some embodiments, TAA 1 is CD24 and TAA 2 is CD72 (or vice versa). In some embodiments, TAA 1 is CD24 and TAA 2 is CD79a (or vice versa). In some embodiments, TAA 1 is CD24 and TAA 2 is CD79b (or vice versa). In some embodiments, TAA 1 is CD40L and TAA 2 is CD72 (or vice versa). In some embodiments, TAA 1 is CD40L and TAA 2 is CD79a (or vice versa). In some embodiments, TAA 1 is CD40L and TAA 2 is CD79b (or vice versa). In some embodiments, TAA 1 is CD72 and TAA 2 is CD79a (or vice versa). In some embodiments, TAA 1 is CD72 and TAA 2 is CD79b (or vice versa). In some embodiments, TAA 1 is CD79a and TAA 2 is CD79b (or vice versa). In some embodiments, TAA 1 and TAA 2 are the same TAA, which is selected from CD19, CD20, CD22, CD123, BCMA, CD33, CLL-1, CD138, CS1, CD38, CD133, FLT3, CD52, TNFRSF13C, TNFRSF13B, CXCR4, PD-L1, LY9, CD200, FCGR2B, CD21, CD23, CD24, CD40L, CD72, CD79a, and CD79b. In some embodiments, TAA 1 is CD19 and TAA 2 is ENPP1 (or vice versa). In some embodiments, TAA 1 is CD19 and TAA 2 is CD74 (or vice versa). In some embodiments, TAA 1 is CD19 and TAA 2 is CD93 (or vice versa). In some embodiments, TAA 1 is CD19 and TAA 2 is CD99 (or vice versa). In some embodiments, TAA 1 is CD20 and TAA 2 is ENPP1 (or vice versa). In some embodiments, TAA 1 is CD20 and TAA 2 is CD74 (or vice versa). In some embodiments, TAA 1 is CD20 and TAA 2 is CD93 (or vice versa). In some embodiments, TAA 1 is CD20 and TAA 2 is CD99 (or vice versa). In some embodiments, TAA 1 is CD22 and TAA 2 is ENPP1 (or vice versa). In some embodiments, TAA 1 is CD22 and TAA 2 is CD74 (or vice versa). In some embodiments, TAA 1 is CD22 and TAA 2 is CD93 (or vice versa). In some embodiments, TAA 1 is CD22 and TAA 2 is CD99 (or vice versa). In some embodiments, TAA 1 is CD123 and TAA 2 is ENPP1 (or vice versa). In some embodiments, TAA 1 is CD123 and TAA 2 is CD74 (or vice versa). In some embodiments, TAA 1 is CD123 and TAA 2 is CD93 (or vice versa). In some embodiments, TAA 1 is CD123 and TAA 2 is CD99 (or vice versa). In some embodiments, TAA 1 is BCMA and TAA 2 is ENPP1 (or vice versa). In some embodiments, TAA 1 is BCMA and TAA 2 is CD74 (or vice versa). In some embodiments, TAA 1 is BCMA and TAA 2 is CD93 (or vice versa). In some embodiments, TAA 1 is BCMA and TAA 2 is CD99 (or vice versa). In some embodiments, TAA 1 is CD33 and TAA 2 is ENPP1 (or vice versa). In some embodiments, TAA 1 is CD33 and TAA 2 is CD74 (or vice versa). In some embodiments, TAA 1 is CD33 and TAA 2 is CD93 (or vice versa). In some embodiments, TAA 1 is CD33 and TAA 2 is CD99 (or vice versa). In some embodiments, TAA 1 is CLL-1 and TAA 2 is ENPP1 (or vice versa). In some embodiments, TAA 1 is CLL-1 and TAA 2 is CD74 (or vice versa). In some embodiments, TAA 1 is CLL-1 and TAA 2 is CD93 (or vice versa). In some embodiments, TAA 1 is CLL-1 and TAA 2 is CD99 (or vice versa). In some embodiments, TAA 1 is CD138 and TAA 2 is ENPP1 (or vice versa). In some embodiments, TAA 1 is CD138 and TAA 2 is CD74 (or vice versa). In some embodiments, TAA 1 is CD138 and TAA 2 is CD93 (or vice versa). In some embodiments, TAA 1 is CD138 and TAA 2 is CD99 (or vice versa). In some embodiments, TAA 1 is CS1 and TAA 2 is ENPP1 (or vice versa). In some embodiments, TAA 1 is CS1 and TAA 2 is CD74 (or vice versa). In some embodiments, TAA 1 is CS1 and TAA 2 is CD93 (or vice versa). In some embodiments, TAA 1 is CS1 and TAA 2 is CD99 (or vice versa). In some embodiments, TAA 1 is CD38 and TAA 2 is ENPP1 (or vice versa). In some embodiments, TAA 1 is CD38 and TAA 2 is CD74 (or vice versa). In some embodiments, TAA 1 is CD38 and TAA 2 is CD93 (or vice versa). In some embodiments, TAA 1 is CD38 and TAA 2 is CD99 (or vice versa). In some embodiments, TAA 1 is CD133 and TAA 2 is ENPP1 (or vice versa). In some embodiments, TAA 1 is CD133 and TAA 2 is CD74 (or vice versa). In some embodiments, TAA 1 is CD133 and TAA 2 is CD93 (or vice versa). In some embodiments, TAA 1 is CD133 and TAA 2 is CD99 (or vice versa). In some embodiments, TAA 1 is FLT3 and TAA 2 is ENPP1 (or vice versa). In some embodiments, TAA 1 is FLT3 and TAA 2 is CD74 (or vice versa). In some embodiments, TAA 1 is FLT3 and TAA 2 is CD93 (or vice versa). In some embodiments, TAA 1 is FLT3 and TAA 2 is CD99 (or vice versa). In some embodiments, TAA 1 is CD52 and TAA 2 is ENPP1 (or vice versa). In some embodiments, TAA 1 is CD52 and TAA 2 is CD74 (or vice versa). In some embodiments, TAA 1 is CD52 and TAA 2 is CD93 (or vice versa). In some embodiments, TAA 1 is CD52 and TAA 2 is CD99 (or vice versa). In some embodiments, TAA 1 is TNFRSF13C and TAA 2 is ENPP1 (or vice versa). In some embodiments, TAA 1 is TNFRSF13C and TAA 2 is CD74 (or vice versa). In some embodiments, TAA 1 is TNFRSF13C and TAA 2 is CD93 (or vice versa). In some embodiments, TAA 1 is TNFRSF13C and TAA 2 is CD99 (or vice versa). In some embodiments, TAA 1 is TNFRSF13B and TAA 2 is ENPP1 (or vice versa). In some embodiments, TAA 1 is TNFRSF13B and TAA 2 is CD74 (or vice versa). In some embodiments, TAA 1 is TNFRSF13B and TAA 2 is CD93 (or vice versa). In some embodiments, TAA 1 is TNFRSF13B and TAA 2 is CD99 (or vice versa). In some embodiments, TAA 1 is CXCR4 and TAA 2 is ENPP1 (or vice versa). In some embodiments, TAA 1 is CXCR4 and TAA 2 is CD74 (or vice versa). In some embodiments, TAA 1 is CXCR4 and TAA 2 is CD93 (or vice versa). In some embodiments, TAA 1 is CXCR4 and TAA 2 is CD99 (or vice versa). In some embodiments, TAA 1 is PD-L1 and TAA 2 is ENPP1 (or vice versa). In some embodiments, TAA 1 is PD-L1 and TAA 2 is CD74 (or vice versa). In some embodiments, TAA 1 is PD-L1 and TAA 2 is CD93 (or vice versa). In some embodiments, TAA 1 is PD-L1 and TAA 2 is CD99 (or vice versa). In some embodiments, TAA 1 is LY9 and TAA 2 is ENPP1 (or vice versa). In some embodiments, TAA 1 is LY9 and TAA 2 is CD74 (or vice versa). In some embodiments, TAA 1 is LY9 and TAA 2 is CD93 (or vice versa). In some embodiments, TAA 1 is LY9 and TAA 2 is CD99 (or vice versa). In some embodiments, TAA 1 is CD200 and TAA 2 is ENPP1 (or vice versa). In some embodiments, TAA 1 is CD200 and TAA 2 is CD74 (or vice versa). In some embodiments, TAA 1 is CD200 and TAA 2 is CD93 (or vice versa). In some embodiments, TAA 1 is CD200 and TAA 2 is CD99 (or vice versa). In some embodiments, TAA 1 is FCGR2B and TAA 2 is ENPP1 (or vice versa). In some embodiments, TAA 1 is FCGR2B and TAA 2 is CD74 (or vice versa). In some embodiments, TAA 1 is FCGR2B and TAA 2 is CD93 (or vice versa). In some embodiments, TAA 1 is FCGR2B and TAA 2 is CD99 (or vice versa). In some embodiments, TAA 1 is CD21 and TAA 2 is ENPP1 (or vice versa). In some embodiments, TAA 1 is CD21 and TAA 2 is CD74 (or vice versa). In some embodiments, TAA 1 is CD21 and TAA 2 is CD93 (or vice versa). In some embodiments, TAA 1 is CD21 and TAA 2 is CD99 (or vice versa). In some embodiments, TAA 1 is CD23 and TAA 2 is ENPP1 (or vice versa). In some embodiments, TAA 1 is CD23 and TAA 2 is CD74 (or vice versa). In some embodiments, TAA 1 is CD23 and TAA 2 is CD93 (or vice versa). In some embodiments, TAA 1 is CD23 and TAA 2 is CD99 (or vice versa). In some embodiments, TAA 1 is CD24 and TAA 2 is ENPP1 (or vice versa). In some embodiments, TAA 1 is CD24 and TAA 2 is CD74 (or vice versa). In some embodiments, TAA 1 is CD24 and TAA 2 is CD93 (or vice versa). In some embodiments, TAA 1 is CD24 and TAA 2 is CD99 (or vice versa). In some embodiments, TAA 1 is CD40L and TAA 2 is ENPP1 (or vice versa). In some embodiments, TAA 1 is CD40L and TAA 2 is CD74 (or vice versa). In some embodiments, TAA 1 is CD40L and TAA 2 is CD93 (or vice versa). In some embodiments, TAA 1 is CD40L and TAA 2 is CD99 (or vice versa). In some embodiments, TAA 1 is CD72 and TAA 2 is ENPP1 (or vice versa). In some embodiments, TAA 1 is CD72 and TAA 2 is CD74 (or vice versa). In some embodiments, TAA 1 is CD72 and TAA 2 is CD93 (or vice versa). In some embodiments, TAA 1 is CD72 and TAA 2 is CD99 (or vice versa). In some embodiments, TAA 1 is CD79a and TAA 2 is ENPP1 (or vice versa). In some embodiments, TAA 1 is CD79a and TAA 2 is CD74 (or vice versa). In some embodiments, TAA 1 is CD79a and TAA 2 is CD93 (or vice versa). In some embodiments, TAA 1 is CD79a and TAA 2 is CD99 (or vice versa). In some embodiments, TAA 1 is CD79b and TAA 2 is ENPP1 (or vice versa). In some embodiments, TAA 1 is CD79b and TAA 2 is CD74 (or vice versa). In some embodiments, TAA 1 is CD79b and TAA 2 is CD93 (or vice versa). In some embodiments, TAA 1 is CD79b and TAA 2 is CD99 (or vice versa).

In some combinations of MBMs, TAA 1 and TAA 2 are the same TAA, which is selected from CD19, CD20, CD22, CD123, BCMA, CD33, CLL-1, CD138, CS1, CD38, CD133, FLT3, CD52, TNFRSF13C, TNFRSF13B, CXCR4, PD-L1, LY9, CD200, FCGR2B, CD21, CD23, CD24, CD40L, CD72, CD79a, and CD79b. In some embodiments, TAA 1 and TAA2 are both CD19. In some embodiments, TAA 1 and TAA2 are both CD20. In some embodiments, TAA 1 and TAA2 are both CD22. In some embodiments, TAA 1 and TAA2 are both CD123. In some embodiments, TAA 1 and TAA2 are both BCMA. In some embodiments, TAA 1 and TAA2 are both CD33. In some embodiments, TAA 1 and TAA2 are both CLL1. In some embodiments, TAA 1 and TAA2 are both CD138. In some embodiments, TAA 1 and TAA2 are both CS1. In some embodiments, TAA 1 and TAA2 are both CD38. In some embodiments, TAA 1 and TAA2 are both CD133. In some embodiments, TAA 1 and TAA2 are both FLT3. In some embodiments, TAA 1 and TAA2 are both CD52. In some embodiments, TAA 1 and TAA2 are both TNFRSF13C. In some embodiments, TAA 1 and TAA2 are both TNFRSF13B. In some embodiments, TAA 1 and TAA2 are both CXCR4. In some embodiments, TAA 1 and TAA2 are both PD-L1. In some embodiments, TAA 1 and TAA2 are both LY9. In some embodiments, TAA 1 and TAA2 are both CD200. In some embodiments, TAA 1 and TAA2 are both FCGR2B. In some embodiments, TAA 1 and TAA2 are both CD21. In some embodiments, TAA 1 and TAA2 are both CD23. In some embodiments, TAA 1 and TAA2 are both CD24. In some embodiments, TAA 1 and TAA2 are both CD40L. In some embodiments, TAA 1 and TAA2 are both CD72. In some embodiments, TAA 1 and TAA2 are both CD79a. In some embodiments, TAA 1 and TAA2 are both CD79b.

A TAA ABM can comprise, for example, a ligand- or an antibody-based moiety. For example, in the case of BCMA as a TAA, the ABM can be APRIL, the BCMA ligand, or a portion thereof that binds BCMA, or an anti-BCMA antibody or an antigen-binding fragment thereof. Ligands and antibodies that bind to TAAs are well-known in the art. In the case of antibody-based moieties, the anti-TAA antibody or antigen-binding fragment can comprise, for example, the CDR sequences of an antibody set forth in Table 13 or elsewhere in Section 7.7 (including its subparts). In some embodiments, the anti-TAA antibody or antigen-binding domain thereof has the heavy and light chain variable region sequences of an antibody set forth in Table 13 or elsewhere in Section 7.7 (including its subparts).

TABLE 13
Exemplary Anti-Tumor-Associated Antigen Antibodies

| Target | Examples of Antibody Name and/or Reference(s) and/or Source |
|---|---|
| ALK | antibodies described in e.g., Mino-Kenudson et al., 2010, Clin Cancer Res |
| | 16(5): 1561-1571 |
| B7H3 | MGA271 (Macrogenics) |
| BCMA | Any BCMA antibody described in WO2012163805, WO200112812, or |
| | WO2003062401. |
| CAIX | Antibody clone 303123 (R&D Systems) |
| CD123 | U.S. Pat. No. 8,852,551; EP2426148; WO2014138819; WO2016028896; WO2014130635 |
| CD171 | Hong et al., 2014, J Immunother 37(2): 93-104. |
| CD19 | WO2014031687; WO2012079000; WO2014153270; U.S. Pat. No. 7,741,465; the |
| | CD19 binder of Yescarta or Blinatumomab |
| CD20 | Rituximab, Ofatumumab, Ocrelizumab, Veltuzumab, GA101; any CD20 antibody |
| | described in WO2014047231, WO2016081490, WO2017112762, |
| | WO2017112775, WO2018093821, U.S. Pat. No. 9,657,102 |
| CD21 | ab75985 (Abcam); ab9492 (Abcam); 2G9 (ThermoFisher); HB5 (ThermoFisher); |
| | MAB4909 (R&D Systems) |
| CD22 | Haso et al., 2013, Blood, 121(7): 1165-1174; Wayne et al., 2010, Clin Cancer Res |
| | 16(6): 1894-1903; Kato et al., 2013, Leuk Res 37(1): 83-88; Creative BioMart |
| | (creativebiomart.net): MOM-18047-S(P). |
| CD23 | Any CD23 antibody described in U.S. Pat. No. 7,008,623 or U.S. Pat. No. 6,011,138; |
| | lumiliximab (Biogen); ab16702 (Abcam); SP23 (ThermoFisher) |
| CD24 | Any CD24 antibody described in U.S. Pat. No. 8,614,301; SN3 (ThermoFisher); |
| | SN3b (ThermoFisher); 2Q1282 (Santa Cruz Biotechnology); 3H1143 (Santa Cruz |
| | Biotechnology); ALB9 (Santa Cruz Biotechnology); MAB5248 (R&D Systems) |
| CD30 | Any CD30 antibody described in U.S. Pat. No. 7,090,843 B1, or EP0805871 |
| CD33 | Bross et al., 2001, Clin Cancer Res 7(6): 1490-1496 (Gemtuzumab Ozogamicin, |
| | hP67.6), Caron et al., 1992, Cancer Res 52(24): 6761-6767 (Lintuzumab, |
| | HuM195), Lapusan et al., 2012, Invest New Drugs 30(3): 1121-1131 (AVE9633), |
| | Aigner et al., 2013, Leukemia 27(5): 1107-1115 (AMG330, CD33 BiTE), Dutour et |
| | al., 2012, Adv Hematol 2012: 683065, or Pizzitola et al., 2014, Leukemia |
| | doi: 10.1038/Lue.2014.62. |
| CD38 | Daratumumab (see, e.g., Groen et al., 2010, Blood 116(21): 1261-1262; MOR202 |
| | (see, e.g., U.S. Pat. No. 8,263,746); or any CD38 antibody described in U.S. Pat. |
| | No. 8,362,211. |
| CD40L | Any CD40L antibody described in U.S. Pat. No. 9,228,018, US 2003/0099642, |
| | WO/2013/056068, US 2003/0099642, WO/2016/040571; 24-31 (Biolegend); |
| | ab52750 (Abcam); ab47204 (Abcam); CDP7657 (UCB Pharma); ruplizumab |
| | (Biogen) |
| CD44v6 | Casucci et al., 2013, Blood 122(20): 3461-3472. |
| CD52 | alemtuzumab (Genzyme); ANT1034 (see, Holgate et al., 2015, PLOS ONE 10(9): |
| | e0138123; any CD52 antibody described in WO/2010/132659; any CD52 antibody |
| | described in U.S. Pat. No. 9,708,407; any CD52 antibody described in |
| | WO/2010/132659 |
| ENPP1 | YE1/19.1 (Biolegend); DCABH-11412 (Creative Diagnostics) |
| CD72 | 3F3 (Biolegend); Bu40 (ThermoFisher); H-7 (Santa Cruz Biotechnology); H-96 |
| | (Santa Cruz Biotechnology); G-5 (Santa Cruz Biotechnology); ab92509 (Abcam) |
| CD74 | Any CD74 antibody described in WO/2012/104344; LN2 (Abcam); MAB35901 |
| | (R&D Systems) |
| CD79a | ab62650 (Abcam); ab79414 (Abcam); MAB69201 (R&D Systems); HM57 (Bio- |
| | Rad) |
| CD79b | Any CD79b antibody described in WO 2014/011521; ab130422 (Abcam); |
| | ab134147 (Abcam); polatuzumab (Genentech) |
| CD93 | 273107 (Invitrogen); R139 (Invitrogen); MBS6008585 (MyBioSource); |
| | MBS838462 (MyBioSource); X-2 (Bio-Rad) |
| CD97 | antibodies described in, e.g., U.S. Pat. No. 6,846,911; de Groot et al., 2009, J |
| | Immunol 183(6): 4127-4134; antibody from R&D: MAB373 |
| CD99 | Any CD99 antibody described in WO/2015/161267; O13 (Invitrogen); HO36-1.1 |
| | (Invitrogen); 12E7 (Novus Biologicals) |
| CD133 | Any CD133 antibody described in U.S. Pat. No. 9,624,303, WO 2016/154623, or |
| | WO 2011/089211; 5E3 (ThermoFisher); MAB11331 (R&D Systems); MAB4310 |
| | (Millipore Sigma) |
| CD138 | Any CD138 antibody described in WO/2009/080829, WO/2017/014679, or U.S. |
| | Pat. No. 9,289,509; nBT062 (Biotest AG); MI15, B-A38, SP152, DL-101 |
| | (ThermoFisher) |
| CD200 | Any CD200 antibody described in U.S. Pat. No. 7,887,798; ab23552 (Abcam); |
| | Ox104 (ThermoFisher) |
| CEA | Chmielewski et al., 2012, Gastoenterology 143(4): 1095-1107. |
| CLDN6 | WO2015069794; IMAB027, mAb, Ganymed Pharmaceuticals |
| CLL-1 | PE-CLL1-hu Cat# 353604 (BioLegend); and PE-CLL1 (CLEC12A) Cat# 562566 |
| | (BD); WO 2014/051433 A1; US 2016/0368994 A1; US 2013/0295118 A1; U.S. Pat. |
| | No. 8,536,310 B2; Lu et al., 2014, Angewandte Chemie International Edition |
| | 53(37): 9841-9845; Leong et al., 2017, Blood 129(5): 609-618; WO 2016/014535; |
| | WO 2017/125897 |
| CS1 | Elotuzumab (BMS), see e.g., Tai et al., 2008, Blood 112(4): 1329-37; Tai et al., |
| | 2007, Blood. 110(5): 1656-63. |
| CXCR4 | Any CXCR4 antibody described in U.S. Pat. Nos. 7,138,496, 8,329,178, 8,450,464, |
| | 9,249,223, or 9,260,527 |
| EGFR | Cetuximab, panitumumab, zalutumumab, nimotuzumab, or matuzumab |
| EGFRvIII | WO2012138475; WO2014130657 |
| EPCAM | MT110, EpCAM-CD3 bispecific Ab (see, e.g., |
| | clinicaltrials.gov/ct2/show/NCT00635596); Edrecolomab; 3622W94; ING-1; or |
| | adecatumumab (MT201). |
| EphA2 | Yu et al., 2014, Mol Ther 22(1): 102-111. |
| Ephrin B2 | Abengozar et al., 2012, Blood 119(19): 4565-4576. |
| ERBB2 | Trastuzumab or pertuzumab. |
| (Her2/neu) | |
| FAP | Ostermann et al., 2008, Clinical Cancer Research 14: 4584-4592 (FAP5), US Pat. |
| | Publication No. 2009/0304718; sibrotuzumab (see e.g., Hofheinz et al., 2003, |
| | Oncology Research and Treatment 26(1): 44-48); and Tran et al., 2013, J Exp Med |
| | 210(6): 1125-1135. |
| FCGR2B | Any FCGR2B antibody described in U.S. Pat. No. 8,802,089 or WO 2017/103895; |
| | ab45143 (Abcam); AT130-2 (ThermoFisher); 2E10 (Millipore Sigma) |
| FLT3 | Any FLT3 antibody described in WO2011076922, U.S. Pat No. 5,777,084, |
| | EP0754230, or US20090297529. |
| Folate | IMGN853, or any folate receptor alpha antibody described in US20120009181; |
| receptor | U.S. Pat. No. 4,851,332, LK26: U.S. Pat. No. 5,952,484. |
| alpha | |
| Folate | antibodies described in, e.g., US20100297138; W02007/067992 |
| receptor | |
| beta | |
| GD2 | Mujoo et al., Cancer Res. 47(4): 1098-1104 (1987); Cheung et al., Cancer Res |
| | 45(6): 2642-2649 (1985), Cheung et al., J Clin Oncol 5(9): 1430-1440 (1987), |
| | Cheung et al., J Clin Oncol 16(9): 3053-3060 (1998), Handgretinger et al., Cancer |
| | Immunol Immunother 35(3): 199-204 (1992); |
| | mAb 14.18, 14G2a, ch14.18, hu14.18, 3F8, hu3F8, 3G6, 8B6, 60C3, 10B8, |
| | ME36.1, or 8H9 (see e.g., WO2012033885, WO2013040371, WO2013192294, |
| | WO2013061273, WO2013123061, WO2013074916, and WO201385552). |
| | Any GD2 antibody described in US Publication No.: 20100150910 or PCT |
| | Publication No.: WO 2011160119. |
| GD3 | Any GD3 antibody described in U.S. Pat. No. 7,253,263; U.S. Pat. No. 8,207,308; US |
| | 20120276046; EP1013761; WO2005035577; or U.S. Pat. No. 6,437,098. |
| GloboH | VK9; Kudryashov et al., 1998, Glycoconj J.15(3): 243-9; Lou et al., 2014, Proc Natl |
| | Acad Sci USA 111(7): 2482-2487; MBr1: Bremer et al., 1984, J Biol Chem |
| | 259: 14773-14777. |
| gp100 | HMB45, NKIbetaB, or any anti-gp100 antibody described in WO2013165940, or |
| | US20130295007 |
| GPRC5D | R&Dsystems: FAB6300A; Lifespan Biosciences: LS-A4180 |
| HMWMAA | antibodies described in, e.g., Kmiecik et al., 2014, Oncoimmunology 3(1): e27185 |
| | (PMID: 24575382) (mAb9.2.27); U.S. Pat. No. 6,528,481; WO2010033866; US |
| | 20140004124 |
| IGF-I | Any IGF-I receptor antibody described in U.S. Pat. No. 8,344,112 B2; EP2322550 A1; WO |
| receptor | 2006/138315, or PCT/US2006/022995. |
| IL-11Ra | Abcam (cat# ab55262) or Novus Biologicals (cat# EPR5446) |
| IL-13Ra2 | Any IL-13Ra2 antibody described in WO2008/146911, WO2004087758, or |
| | WO2004087758 |
| KIT | Any KIT antibody described in U.S. Pat. No. 7,915,391, US20120288506 |
| KLRG2 | ab121563 (Abcam); B-12 or sc-514346 (Santa Cruz); HPA018199 (Sigma Aldrich) |
| LewisY | Kelly et al., Cancer Biother Radiopharm 23(4): 411-423 (2008) (hu3S193 Ab |
| | (scFvs)); Dolezal et al., Protein Engineering 16(1): 47-56 (2003) (NC10 scFv) |
| LMP2 | Any LMP2 antibody described in U.S. Pat. No. 7,410,640 or US 2005/0129701 |
| LRP6 | Any LRP6 antibody described in WO2009064944, WO2009056634, |
| | WO2011119661, WO2011138392, WO2011138391, WO2013067355, |
| | WO2014029752, WO2017093478 |
| Mesothelin | Any mesothelin antibody described in US 20110262448, US 2012/0107933 or U.S. Pat. No. |
| | 9,719,996 |
| MUC1 | SAR566658 |
| NCAM | 2-2B: MAB5324 (EMD Millipore) |
| NY-BR-1 | antibodies described in, e.g., Jager et al., 2007, Appl Immunohitochem Mol |
| | Morphol 15(1): 77-83 |
| o-acetyl- | 8B6 |
| GD2 | |
| PDGFR- | Abcam ab32570 |
| beta | |
| PD-L1 | Any PD-L1 antibody described in US 2015/0203580, US 2017/0058033, US |
| | 2017/0204184, U.S. Pat. No. 8,741,295, U.S. Pat. No. 9,789,183, or U.S. Pat. No. |
| | 9,637,546 |
| LY9 | HLy9.25 (e.g., Lifespan Biosciences, Inc. cat. no. LS-C112605); MAB1898 (R&D |
| | Systems) |
| PLAC1 | antibodies described in, e.g., Ghods et al., 2013, Biotechnol Appl Biochem |
| | doi: 10.1002/bab.1177 |
| Polysialic | antibodies described in e.g., Nagae et al., 2013, J Biol Chem 288(47): 33784- |
| acid | 33796 |
| PRSS21 | Any PRSS21 antibody described in U.S. Pat. No.: 8,080,650. |
| PSCA | Morgenroth et al., Prostate 67(10): 1121-1131 (2007) (scFv 7F5); Nejatollahi et al., |
| | J of Oncology 2013(2013), article ID 839831 (scFv C5-II); or any PSCA antibody |
| | described in US Pat Publication No. 20090311181. |
| PSMA | Parker et al., Protein Expr Purif 89(2): 136-145 (2013), US 20110268656 (J591 |
| | ScFv); Frigerio et al, European J Cancer 49(9): 2223-2232 (2013) (scFvD2B); WO |
| | 2006125481 (mAbs 3/A12, 3/E7 and 3/F11) or single chain antibody fragments |
| | (scFv A5 and D7). |
| ROR1 | Hudecek et al., Clin Cancer Res 19(12): 3153-3164 (2013); or any ROR1 antibody |
| | described in WO 2011159847 or US20130101607. |
| SSEA-4 | MC813 (Cell Signaling) |
| TAG72 | Hornbach et al., Gastroenterology 113(4): 1163-1170 (1997) or Abcam ab691. |
| TEM1/CD248 | antibodies described in, e.g., Marty et al., 2006, Cancer Lett 235(2): 298-308; Zhao |
| | et al., 2011, J Immunol Methods 363(2): 221-232 |
| Tn | Brooks et al., PNAS 107(22): 10056-10061 (2010); Stone et al., OncoImmunology |
| | 1(6): 863-873(2012); any Tn antibody described in U.S. Pat. No. 8,440,798 |
| TNFRSF13C | Any TNFRSF13C antibody described in WO 2010/007082, U.S. Pat. No. 9,382,326 |
| TNFRSF13B | Any TNFRSF13B antibody described in WO 2004/011611; LS-C89973 (Lifespan |
| | Biosciences, Inc.) M02952-1 (Boster Biological Technology); MAB1041, |
| | MAB1741, and MAB174 (R&D Systems) |
| TSHR | antibodies described in, e.g., Marty et al., 2006, Cancer Lett235(2): 298-308; Zhao |
| | et al., 2011, J Immunol Methods 363(2): 221-232 |
| Tyrosinase | Any tyrosinase antibody described in U.S. Pat. No. 5,843,674 or U.S. Pat. No. 19,950,504,048. |
| VEGFR2 | Chinnasamy et al., J Clin Invest 120(11): 3953-3968 (2010). |

### 7.7.1. BCMA

In certain aspects, the present disclosure provides MBMs and combinations of MBMs in which ABM2 and/or ABM5 binds specifically to BCMA. BCMA is a tumor necrosis family receptor (TNFR) member expressed on cells of the B-cell lineage. BCMA expression is the highest on terminally differentiated B cells that assume the long lived plasma cell fate, including plasma cells, plasmablasts and a subpopulation of activated B cells and memory B cells. BCMA is involved in mediating the survival of plasma cells for maintaining long-term humoral immunity. The expression of BCMA has been recently linked to a number of cancers, autoimmune disorders, and infectious diseases. Cancers with increased expression of BCMA include some hematological cancers, such as multiple myeloma, Hodgkin's and non-Hodgkin's lymphoma, various leukemias, and glioblastoma.

MBMs comprising a ABM that binds to BCMA can comprise, for example, an anti-BCMA antibody or an antigen-binding domain thereof. The anti-BCMA antibody or antigen-binding domain thereof can comprise, for example, CDR, VH, VL, or scFV sequences set forth in Tables 14A-14G (collectively, “Table 14”).

TABLE 14A
BCMA Binders-Variable domain and scFv sequences

| | | | SEQ |
|---|---|---|---|
| | | | ID |
| Binder | Domain | Sequence | NO. |
| BCMA-1 | VH | EVQLVESGGGLVQPGGSLRLSCAVSGFALSNHGMSWVRRAPGKGL | 333 |
| | | EWVSGIVYSGSTYYAASVKGRFTISRDNSRNTLYLQMNSLRPEDTAIY | |
| | | YCSAHGGESDVWGQGTTVTVSS | |
| | VL | DIQLTQSPSSLSASVGDRVTITCRASQSISSYLNWYQQKPGKAPKLLI | 334 |
| | | YAASSLQSGVPSRFSGSGSGTDFTLTISSLQPEDFATYYCQQSYSTP | |
| | | YTFGQGTKVEIK | |
| | scFv | EVQLVESGGGLVQPGGSLRLSCAVSGFALSNHGMSWVRRAPGKGL | 335 |
| | | EWVSGIVYSGSTYYAASVKGRFTISRDNSRNTLYLQMNSLRPEDTAIY | |
| | | YCSAHGGESDVWGQGTTVTVSSASGGGGSGGRASGGGGSDIQLT | |
| | | QSPSSLSASVGDRVTITCRASQSISSYLNWYQQKPGKAPKLLIYAASS | |
| | | LQSGVPSRFSGSGSGTDFTLTISSLQPEDFATYYCQQSYSTPYTFGQ | |
| | | GTKVEIK | |
| BCMA-2 | VH | QVQLVESGGGLVQPGRSLRLSCAASGFTFSNYAMSWVRQAPGKGL | 336 |
| | | GWWVSGISRSGENTYYADSVKGRFTISRDNSKNTLYLQMNSLRDEDTA | |
| | | VYYCARSPAHYYGGMDVWGQGTTVTVSS | |
| | VL | DIVLTQSPGTLSLSPGERATLSCRASQSISSSFLAWYQQKPGQAPRL | 337 |
| | | LIYGASRRATGIPDRFSGSGSGTDFTLTISRLEPEDSAVYYCQQYHSS | |
| | | PSWTFGQGTKLEIK | |
| | scFv | QVQLVESGGGLVQPGRSLRLSCAASGFTFSNYAMSWVRQAPGKGL | 338 |
| | | GWWVSGISRSGENTYYADSVKGRFTISRDNSKNTLYLQMNSLRDEDTA | |
| | | VYYCARSPAHYYGGMDVWGQGTTVTVSSASGGGGSGGRASGGGG | |
| | | SDIVLTQSPGTLSLSPGERATLSCRASQSISSSFLAWYQQKPGQAPR | |
| | | LLIYGASRRATGIPDRFSGSGSGTDFTLTISRLEPEDSAVYYCQQYHS | |
| | | SPSWTFGQGTKLEIK | |
| BCMA-3 | VH | QVQLVESGGGLVQPGRSLRLSCAASGFTFDDYAMHWVRQAPGKGL | 339 |
| | | EWVSGISWNSGSIGYADSVKGRFTISRDNAKNSLYLQMNSLRAEDTA | |
| | | LYYCSVHSFLAYWGQGTLVTVSS | |
| | VL | DIVMTQTPLSLPVTPGEPASISCRSSQSLLHSNGYNYLDWYLQKPGQ | 340 |
| | | SPQLLIYLGSNRASGVPDRFSGSGSGTDFTLKISRVEAEDVGVYYCM | |
| | | QALQTPYTFGQGTKVEIK | |
| | scFv | QVQLVESGGGLVQPGRSLRLSCAASGFTFDDYAMHWVRQAPGKGL | 341 |
| | | EWVSGISWNSGSIGYADSVKGRFTISRDNAKNSLYLQMNSLRAEDTA | |
| | | LYYCSVHSFLAYWGQGTLVTVSSASGGGGSGGRASGGGGSDIVMT | |
| | | QTPLSLPVTPGEPASISCRSSQSLLHSNGYNYLDWYLQKPGQSPQLL | |
| | | IYLGSNRASGVPDRFSGSGSGTDFTLKISRVEAEDVGVYYCMQALQT | |
| | | PYTFGQGTKVEIK | |
| BCMA-4 | VH | EVQLLESGGGLVQPGGSLRLSCAVSGFALSNHGMSWVRRAPGKGL | 342 |
| | | EWVSGIVYSGSTYYAASVKGRFTISRDNSRNTLYLQMNSLRPEDTAIY | |
| | | YCSAHGGESDVWGQGTTVTVSS | |
| | VL | DIVMTQTPLSLSVTPGQPASISCKSSQSLLRNDGKTPLYWYLQKAGQ | 343 |
| | | PPQLLIYEVSNRFSGVPDRFSGSGSGTDFTLKISRVEAEDVGAYYCM | |
| | | QNIQFPSFGGGTKLEIK | |
| | scFv | EVQLLESGGGLVQPGGSLRLSCAVSGFALSNHGMSWVRRAPGKGL | 344 |
| | | EWVSGIVYSGSTYYAASVKGRFTISRDNSRNTLYLQMNSLRPEDTAIY | |
| | | YCSAHGGESDVWGQGTTVTVSSASGGGGSGGRASGGGGSDIVMT | |
| | | QTPLSLSVTPGQPASISCKSSQSLLRNDGKTPLYWYLQKAGQPPQLL | |
| | | IYEVSNRFSGVPDRFSGSGSGTDFTLKISRVEAEDVGAYYCMQNIQF | |
| | | PSFGGGTKLEIK | |
| BCMA-5 | VH | QVQLVQSGAEVRKTGASVKVSCKASGYIFDNFGINWVRQAPGQGLE | 345 |
| | | WMGWINPKNNNTNYAQKFQGRVTITADESTNTAYMEVSSLRSEDTA | |
| | | VYYCARGPYYYQSYMDVWGQGTMVTVSS | |
| | VL | DIVMTQTPLSLPVTPGEPASISCRSSQSLLHSNGYNYLNWYLQKPGQ | 346 |
| | | SPQLLIYLGSKRASGVPDRFSGSGSGTDFTLHITRVGAEDVGVYYCM | |
| | | QALQTPYTFGQGTKLEIK | |
| | scFv | QVQLVQSGAEVRKTGASVKVSCKASGYIFDNFGINWVRQAPGQGLE | 347 |
| | | WMGWINPKNNNTNYAQKFQGRVTITADESTNTAYMEVSSLRSEDTA | |
| | | VYYCARGPYYYQSYMDVWGQGTMVTVSSASGGGGSGGRASGGG | |
| | | GSDIVMTQTPLSLPVTPGEPASISCRSSQSLLHSNGYNYLNWYLQKP | |
| | | GQSPQLLIYLGSKRASGVPDRFSGSGSGTDFTLHITRVGAEDVGVYY | |
| | | CMQALQTPYTFGQGTKLEIK | |
| BCMA-6 | VH | QVQLQESGGGLVQPGGSLRLSCAASGFTFSSDAMTWVRQAPGKGL | 348 |
| | | EWVSVISGSGGTTYYADSVKGRFTISRDNSKNTLYLQMNSLRAEDTA | |
| | | VYYCAKLDSSGYYYARGPRYWGQGTLVTVSS | |
| | VL | DIQLTQSPSSLSASVGDRVTITCRASQSISSYLNWYQQKPGKAPKLLI | 349 |
| | | YGASTLASGVPARFSGSGSGTHFTLTINSLQSEDSATYYCQQSYKRA | |
| | | SFGQGTKVEIK | |
| | scFv | QVQLQESGGGLVQPGGSLRLSCAASGFTFSSDAMTWVRQAPGKGL | 350 |
| | | EWVSVISGSGGTTYYADSVKGRFTISRDNSKNTLYLQMNSLRAEDTA | |
| | | VYYCAKLDSSGYYYARGPRYWGQGTLVTVSSASGGGGSGGRASG | |
| | | GGGSDIQLTQSPSSLSASVGDRVTITCRASQSISSYLNWYQQKPGKA | |
| | | PKLLIYGASTLASGVPARFSGSGSGTHFTLTINSLQSEDSATYYCQQS | |
| | | YKRASFGQGTKVEIK | |
| BCMA-7 | VH | QVQLVQSGAEVKKPGASVKVSCKASGYTFSNYGITWVRQAPGQGLE | 351 |
| | | WMGWISAYNGNTNYAQKFQGRVTMTRNTSISTAYMELSSLRSEDTA | |
| | | VYYCARGPYYYYMDVWGKGTMVTVSS | |
| | VL | EIVMTQSPLSLPVTPGEPASISCRSSQSLLYSNGYNYVDWYLQKPGQ | 352 |
| | | SPQLLIYLGSNRASGVPDRFSGSGSGTDFKLQISRVEAEDVGIYYCM | |
| | | QGRQFPYSFGQGTKVEIK | |
| | scFv | QVQLVQSGAEVKKPGASVKVSCKASGYTFSNYGITWVRQAPGQGLE | 353 |
| | | WMGWISAYNGNTNYAQKFQGRVTMTRNTSISTAYMELSSLRSEDTA | |
| | | VYYCARGPYYYYMDVWGKGTMVTVSSASGGGGSGGRASGGGGSE | |
| | | IVMTQSPLSLPVTPGEPASISCRSSQSLLYSNGYNYVDWYLQKPGQS | |
| | | PQLLIYLGSNRASGVPDRFSGSGSGTDFKLQISRVEAEDVGIYYCMQ | |
| | | GRQFPYSFGQGTKVEIK | |
| BCMA-8 | VH | EVQLLETGGGLVQPGGSLRLSCAVSGFALSNHGMSWVRRAPGKGL | 354 |
| | | EWVSGIVYSGSTYYAASVKGRFTISRDNSRNTLYLQMNSLRPEDTAIY | |
| | | YCSAHGGESDVWGQGTTVTVSS | |
| | VL | EIVLTQSPATLSVSPGESATLSCRASQSVSSNLAWYQQKPGQAPRLL | 355 |
| | | IYGASTRASGIPDRFSGSGSGTDFTLTISSLQAEDVAVYYCQQYGSSL | |
| | | TFGGGTKVEIK | |
| | scFv | EVQLLETGGGLVQPGGSLRLSCAVSGFALSNHGMSWVRRAPGKGL | 356 |
| | | EWVSGIVYSGSTYYAASVKGRFTISRDNSRNTLYLQMNSLRPEDTAIY | |
| | | YCSAHGGESDVWGQGTTVTVSSASGGGGSGGRASGGGGSEIVLTQ | |
| | | SPATLSVSPGESATLSCRASQSVSSNLAWYQQKPGQAPRLLIYGAST | |
| | | RASGIPDRFSGSGSGTDFTLTISSLQAEDVAVYYCQQYGSSLTFGGG | |
| | | TKVEIK | |
| BCMA-9 | VH | EVQLVETGGGLVQPGGSLRLSCAVSGFALSNHGMSWVRRAPGKGL | 357 |
| | | EWVSGIVYSGSTYYAASVKGRFTISRDNSRNTLYLQMNSLRPEDTAIY | |
| | | YCSAHGGESDVWGQGTTVTVSS | |
| | VL | EIVMTQSPATLSVSPGERATLSCRASQSVSSKLAWYQQKPGQAPRL | 358 |
| | | LMYGASIRATGIPDRFSGSGSGTEFTLTISSLEPEDFAVYYCQQYGSS | |
| | | SWTFGQGTKVEIK | |
| | scFv | EVQLVETGGGLVQPGGSLRLSCAVSGFALSNHGMSWVRRAPGKGL | 359 |
| | | EWVSGIVYSGSTYYAASVKGRFTISRDNSRNTLYLQMNSLRPEDTAIY | |
| | | YCSAHGGESDVWGQGTTVTVSSASGGGGSGGRASGGGGSEIVMT | |
| | | QSPATLSVSPGERATLSCRASQSVSSKLAWYQQKPGQAPRLLMYGA | |
| | | SIRATGIPDRFSGSGSGTEFTLTISSLEPEDFAVYYCQQYGSSSWTFG | |
| | | QGTKVEIK | |
| BCMA-10 | VH | EVQLVETGGGVVQPGGSLRLSCAVSGFALSNHGMSWWRRAPGKGL | 360 |
| | | EWVSGIVYSGSTYYAASVKGRFTISRDNSRNTLYLQMNSLRPEDTAIY | |
| | | YCSAHGGESDVWGQGTTVTVSS | |
| | VL | EIVLTQSPGTLSLSPGERATLSCRASQSVGSTNLAWYQQKPGQAPRL | 361 |
| | | LIYDASNRATGIPDRFSGGGSGTDFTLTISRLEPEDFAVYYCQQYGSS | |
| | | PPWTFGQGTKVEIK | |
| | scFv | EVQLVETGGGVVQPGGSLRLSCAVSGFALSNHGMSWWRRAPGKGL | 362 |
| | | EWVSGIVYSGSTYYAASVKGRFTISRDNSRNTLYLQMNSLRPEDTAIY | |
| | | YCSAHGGESDVWGQGTTVTVSSASGGGGSGGRASGGGGSEIVLTQ | |
| | | SPGTLSLSPGERATLSCRASQSVGSTNLAWYQQKPGQAPRLLIYDAS | |
| | | NRATGIPDRFSGGGSGTDFTLTISRLEPEDFAVYYCQQYGSSPPWTF | |
| | | GQGTKVEIK | |
| BCMA-11 | VH | QVQLVESGGGLVKPGGSLRLSCAASGFTFSDYYMSWIRQAPGKGLE | 363 |
| | | WVSYISSSGSTIYYADSVKGRFTISRDNAKNSLYLQMNSLRAEDTAVY | |
| | | YCARESGDGMDVWGQGTTVTVSS | |
| | VL | DIQMTQSPSSLSASVGDRVTITCRASQSISSYLNWYQQKPGKAPKLLI | 364 |
| | | YAASSLQSGVPSRFSGSGSGTDFTLTISSLQPEDFATYYCQQSYTLA | |
| | | FGQGTKVDIK | |
| | scFv | QVQLVESGGGLVKPGGSLRLSCAASGFTFSDYYMSWIRQAPGKGLE | 365 |
| | | WVSYISSSGSTIYYADSVKGRFTISRDNAKNSLYLQMNSLRAEDTAVY | |
| | | YCARESGDGMDVWGQGTTVTVSSASGGGGSGGRASGGGGSDIQM | |
| | | TQSPSSLSASVGDRVTITCRASQSISSYLNWYQQKPGKAPKLLIYAAS | |
| | | SLQSGVPSRFSGSGSGTDFTLTISSLQPEDFATYYCQQSYTLAFGQG | |
| | | TKVDIK | |
| BCMA-12 | VH | QVQLVQSGGGLVKPGGSLRLSCAASGFTFSDYYMSWIRQAPGKGLE | 366 |
| | | WVSYISSSGNTIYYADSVKGRFTISRDNAKNSLYLQMNSLRAEDTAVY | |
| | | YCARSTMVREDYWGQGTLVTVSS | |
| | VL | DIVLTQSPLSLPVTLGQPASISCKSSESLVHNSGKTYLNWFHQRPGQ | 367 |
| | | SPRRLIYEVSNRDSGVPDRFTGSGSGTDFTLKISRVEAEDVGVYYCM | |
| | | QGTHWPGTFGQGTKLEIK | |
| | scFv | QVQLVQSGGGLVKPGGSLRLSCAASGFTFSDYYMSWIRQAPGKGLE | 368 |
| | | WVSYISSSGNTIYYADSVKGRFTISRDNAKNSLYLQMNSLRAEDTAVY | |
| | | YCARSTMVREDYWGQGTLVTVSSASGGGGSGGRASGGGGSDIVLT | |
| | | QSPLSLPVTLGQPASISCKSSESLVHNSGKTYLNWFHQRPGQSPRRL | |
| | | IYEVSNRDSGVPDRFTGSGSGTDFTLKISRVEAEDVGVYYCMQGTH | |
| | | WPGTFGQGTKLEIK | |
| BCMA-13 | VH | QVQLVESGGGLVQPGGSLRLSCAVSGFALSNHGMSWVRRAPGKGL | 369 |
| | | EWVSGIVYSGSTYYAASVKGRFTISRDNSRNTLYLQMNSLRPEDTAIY | |
| | | YCSAHGGESDVWGQGTTVTVSS | |
| | VL | DIRLTQSPSPLSASVGDRVTITCQASEDINKFLNWYHQTPGKAPKLLIY | 370 |
| | | DASTLQTGVPSRFSGSGSGTDFTLTINSLQPEDIGTYYCQQYESLPLT | |
| | | FGGGTKVEIK | |
| | scFv | QVQLVESGGGLVQPGGSLRLSCAVSGFALSNHGMSWVRRAPGKGL | 371 |
| | | EWVSGIVYSGSTYYAASVKGRFTISRDNSRNTLYLQMNSLRPEDTAIY | |
| | | YCSAHGGESDVWGQGTTVTVSSASGGGGSGGRASGGGGSDIRLT | |
| | | QSPSPLSASVGDRVTITCQASEDINKFLNWYHQTPGKAPKLLIYDAST | |
| | | LQTGVPSRFSGSGSGTDFTLTINSLQPEDIGTYYCQQYESLPLTFGG | |
| | | GTKVEIK | |
| BCMA-14 | VH | EVQLVETGGGLVQPGGSLRLSCAVSGFALSNHGMSWVRRAPGKGL | 357 |
| | | EWVSGIVYSGSTYYAASVKGRFTISRDNSRNTLYLQMNSLRPEDTAIY | |
| | | YCSAHGGESDVWGQGTTVTVSS | |
| | VL | ETTLTQSPATLSVSPGERATLSCRASQSVGSNLAWYQQKPGQGPRL | 372 |
| | | LIYGASTRATGIPARFSGSGSGTEFTLTISSLQPEDFAVYYCQQYNDW | |
| | | LPVTFGQGTKVEIK | |
| | scFv | EVQLVETGGGLVQPGGSLRLSCAVSGFALSNHGMSWVRRAPGKGL | 373 |
| | | EWVSGIVYSGSTYYAASVKGRFTISRDNSRNTLYLQMNSLRPEDTAIY | |
| | | YCSAHGGESDVWGQGTTVTVSSASGGGGSGGRASGGGGSETTLT | |
| | | QSPATLSVSPGERATLSCRASQSVGSNLAWYQQKPGQGPRLLIYGA | |
| | | STRATGIPARFSGSGSGTEFTLTISSLQPEDFAVYYCQQYNDWLPVT | |
| | | FGQGTKVEIK | |
| BCMA-15 | VH | EVQLVESGGGLVQPGGSLRLSCAVSGFALSNHGMSWVRRAPGKGL | 333 |
| | | EWVSGIVYSGSTYYAASVKGRFTISRDNSRNTLYLQMNSLRPEDTAIY | |
| | | YCSAHGGESDVWGQGTTVTVSS | |
| | VL | EIVLTQSPGTLSLSPGERATLSCRASQSIGSSSLAWYQQKPGQAPRL | 374 |
| | | LMYGASSRASGIPDRFSGSGSGTDFTLTISRLEPEDFAVYYCQQYAG | |
| | | SPPFTFGQGTKVEIK | |
| | scFv | EVQLVESGGGLVQPGGSLRLSCAVSGFALSNHGMSWVRRAPGKGL | 375 |
| | | EWVSGIVYSGSTYYAASVKGRFTISRDNSRNTLYLQMNSLRPEDTAIY | |
| | | YCSAHGGESDVWGQGTTVTVSSASGGGGSGGRASGGGGSEIVLTQ | |
| | | SPGTLSLSPGERATLSCRASQSIGSSSLAWYQQKPGQAPRLLMYGA | |
| | | SSRASGIPDRFSGSGSGTDFTLTISRLEPEDFAVYYCQQYAGSPPFT | |
| | | FGQGTKVEIK | |
| BCMA-16 | VH | QVQLQESGPGLVKPSETLSLTCTVSGGSISSSYYYWGWIRQPPGKG | 376 |
| | | LEWIGSIYYSGSAYYNPSLKSRVTISVDTSKNQFSLRLSSVTAADTAV | |
| | | YYCARHWQEWPDAFDIWGQGTMVTVSS | |
| | VL | ETTLTQSPAFMSATPGDKVIISCKASQDIDDAMNWYQQKPGEAPLFII | 377 |
| | | QSATSPVPGIPPRFSGSGFGTDFSLTINNIESEDAAYYFCLQHDNFPL | |
| | | TFGQGTKLEIK | |
| | scFv | QVQLQESGPGLVKPSETLSLTCTVSGGSISSSYYYWGWIRQPPGKG | 378 |
| | | LEWIGSIYYSGSAYYNPSLKSRVTISVDTSKNQFSLRLSSVTAADTAV | |
| | | YYCARHWQEWPDAFDIWGQGTMVTVSSGGGGSGGGGSGGGGSE | |
| | | TTLTQSPAFMSATPGDKVIISCKASQDIDDAMNWYQQKPGEAPLFIIQ | |
| | | SATSPVPGIPPRFSGSGFGTDFSLTINNIESEDAAYYFCLQHDNFPLT | |
| | | FGQGTKLEIK | |
| BCMA-17 | VH | QVNLRESGPALVKPTQTLTLTCTFSGFSLRTSGMCVSWIRQPPGKAL | 379 |
| | | EWLARIDWDEDKFYSTSLKTRLTISKDTSDNQVVLRMTNMDPADTAT | |
| | | YYCARSGAGGTSATAFDIWGPGTMVTVSS | |
| | VL | DIQMTQSPSSLSASVGDRVTITCRASQDIYNNLAWFQLKPGSAPRSL | 380 |
| | | MYAANKSQSGVPSRFSGSASGTDFTLTISSLQPEDFATYYCQHYYRF | |
| | | PYSFGQGTKLEIK | |
| | scFv | VNLRESGPALVKPTQTLTLTCTFSGFSLRTSGMCVSWIRQPPGKALE | 381 |
| | | WLARIDWDEDKFYSTSLKTRLTISKDTSDNQVVLRMTNMDPADTATY | |
| | | YCARSGAGGTSATAFDIWGPGTMVTVSSGGGGSGGGGSGGGGSDI | |
| | | QMTQSPSSLSASVGDRVTITCRASQDIYNNLAWFQLKPGSAPRSLMY | |
| | | AANKSQSGVPSRFSGSASGTDFTLTISSLQPEDFATYYCQHYYRFPY | |
| | | SFGQGTKLEIK | |
| BCMA-18 | VH | EVQLVESGGGLVKPGGSLRLSCAASGFTFSSYSMNWVRQAPGKGL | 382 |
| | | EWVSSISSSSSYIYYADSVKGRFTISRDNAKNSLYLQMNSLRAEDTAV | |
| | | YYCAKTIAAVYAFDIWGQGTTVTVSS | |
| | VL | EIVLTQSPLSLPVTPEEPASISCRSSQSLLHSNGYNYLDWYLQKPGQS | 383 |
| | | PQLLIYLGSNRASGVPDRFSGSGSGTDFTLKISRVEAEDVGVYYCMQ | |
| | | ALQTPYTFGQGTKLEIK | |
| | scFv | EVQLVESGGGLVKPGGSLRLSCAASGFTFSSYSMNWVRQAPGKGL | 384 |
| | | EWVSSISSSSSYIYYADSVKGRFTISRDNAKNSLYLQMNSLRAEDTAV | |
| | | YYCAKTIAAVYAFDIWGQGTTVTVSSGGGGSGGGGSGGGGSEIVLT | |
| | | QSPLSLPVTPEEPASISCRSSQSLLHSNGYNYLDWYLQKPGQSPQLL | |
| | | IYLGSNRASGVPDRFSGSGSGTDFTLKISRVEAEDVGVYYCMQALQT | |
| | | PYTFGQGTKLEIK | |
| BCMA-19 | VH | EVQLVESGGGLVKPGGSLRLSCAASGFTFSDYYMSWIRQAPGKGLE | 385 |
| | | WVSYISSSGSTIYYADSVKGRFTISRDNAKNSLYLQMNSLRAEDTAVY | |
| | | YCARDLRGAFDIWGQGTMVTVSS | |
| | VL | SYVLTQSPSVSAAPGYTATISCGGNNIGTKSVHWYQQKPGQAPLLVI | 386 |
| | | RDDSVRPSKIPGRFSGSNSGNMATLTISGVQAGDEADFYCQVWDSD | |
| | | SEHWFGGGTKLTVL | |
| | scFv | EVQLVESGGGLVKPGGSLRLSCAASGFTFSDYYMSWIRQAPGKGLE | 387 |
| | | WVSYISSSGSTIYYADSVKGRFTISRDNAKNSLYLQMNSLRAEDTAVY | |
| | | YCARDLRGAFDIWGQGTMVTVSSGGGGSGGGGSGGGGSSYVLTQ | |
| | | SPSVSAAPGYTATISCGGNNIGTKSVHWYQQKPGQAPLLVIRDDSVR | |
| | | PSKIPGRFSGSNSGNMATLTISGVQAGDEADFYCQVWDSDSEHVVF | |
| | | GGGTKLTVL | |
| BCMA-20 | VH | QVQLVQSGAEVKKPGASVKVSCKPSGYTVTSHYIHWVRRAPGQGLE | 388 |
| | | WMGMINPSGGVTAYSQTLQGRVTMTSDTSSSTVYMELSSLRSEDTA | |
| | | MYYCAREGSGSGWYFDFWGRGTLVTVSS | |
| | VL | SYVLTQPPSVSVSPGQTASITCSGDGLSKKYVSWYQQKAGQSPVVLI | 389 |
| | | SRDKERPSGIPDRFSGSNSADTATLTISGTQAMDEADYYCQAWDDT | |
| | | TVVFGGGTKLTVL | |
| | scFv | QVQLVQSGAEVKKPGASVKVSCKPSGYTVTSHYIHWVRRAPGQGLE | 390 |
| | | WMGMINPSGGVTAYSQTLQGRVTMTSDTSSSTVYMELSSLRSEDTA | |
| | | MYYCAREGSGSGWYFDFWGRGTLVTVSSGGGGSGGGGSGGGGS | |
| | | SYVLTQPPSVSVSPGQTASITCSGDGLSKKYVSWYQQKAGQSPVVLI | |
| | | SRDKERPSGIPDRFSGSNSADTATLTISGTQAMDEADYYCQAWDDT | |
| | | TVVFGGGTKLTVL | |
| BCMA-21 | VH | QVQLQESGPGLVKPSQTLSLTCTVSGGSISSGGYYWSWIRQHPGKG | 391 |
| | | LEWIGYIYYSGSTYYNPSLKSRVTISVDTSKNQFSLKLSSVTAADTAVY | |
| | | YCARAGIAARLRGAFDIWGQGTMVTVSS | |
| | VL | DIVMTQSPSSVSASVGDRVIITCRASQGIRNWLAWYQQKPGKAPNLLI | 392 |
| | | YAASNLQSGVPSRFSGSGSGADFTLTISSLQPEDVATYYCQKYNSAP | |
| | | FTFGPGTKVDIK | |
| | scFv | QVQLQESGPGLVKPSQTLSLTCTVSGGSISSGGYYWSWIRQHPGKG | 393 |
| | | LEWIGYIYYSGSTYYNPSLKSRVTISVDTSKNQFSLKLSSVTAADTAVY | |
| | | YCARAGIAARLRGAFDIWGQGTMVTVSSGGGGSGGGGSGGGGSDI | |
| | | VMTQSPSSVSASVGDRVIITCRASQGIRNWLAWYQQKPGKAPNLLIY | |
| | | AASNLQSGVPSRFSGSGSGADFTLTISSLQPEDVATYYCQKYNSAPF | |
| | | TFGPGTKVDIK | |
| BCMA-22 | VH | QVQLVQSGAEVKKPGSSVKVSCKASGGTFSSYAISWVRQAPGQGLE | 394 |
| | | WMGGIIPIFGTANYAQKFQGRVTITADESTSTAYMELSSLRSEDTAVY | |
| | | YCARRGGYQLLRWDVGLLRSAFDIWGQGTMVTVSS | |
| | VL | SYVLTQPPSVSVAPGQTARITCGGNNIGSKSVHWYQQKPGQAPVLV | 395 |
| | | LYGKNNRPSGVPDRFSGSRSGTTASLTITGAQAEDEADYYCSSRDS | |
| | | SGDHLRVFGTGTKVTVL | |
| | scFv | QVQLVQSGAEVKKPGSSVKVSCKASGGTFSSYAISWVRQAPGQGLE | 396 |
| | | WMGGIIPIFGTANYAQKFQGRVTITADESTSTAYMELSSLRSEDTAVY | |
| | | YCARRGGYQLLRWDVGLLRSAFDIWGQGTMVTVSSGGGGSGGGG | |
| | | SGGGGSSYVLTQPPSVSVAPGQTARITCGGNNIGSKSVHWYQQKPG | |
| | | QAPVLVLYGKNNRPSGVPDRFSGSRSGTTASLTITGAQAEDEADYYC | |
| | | SSRDSSGDHLRVFGTGTKVTVL | |
| BCMA-23 | VH | EVQLQQSGPGLVKPSQTLSLTCAISGDSVSSNSAAWNWIRQSPSRG | 397 |
| | | LEWLGRTYYRSKWYSFYAISLKSRIIINPDTSKNQFSLQLKSVTPEDTA | |
| | | VYYCARSSPEGLFLYWFDPWGQGTLVTVSS | |
| | VL | SSELTQDPAVSVALGQTIRITCQGDSLGNYYATWYQQKPGQAPVLVI | 398 |
| | | YGTNNRPSGIPDRFSASSSGNTASLTITGAQAEDEADYYCNSRDSSG | |
| | | HHLLFGTGTKVTVL | |
| | ScFv | EVQLQQSGPGLVKPSQTLSLTCAISGDSVSSNSAAWNWIRQSPSRG | 399 |
| | | LEWLGRTYYRSKWYSFYAISLKSRIIINPDTSKNQFSLQLKSVTPEDTA | |
| | | VYYCARSSPEGLFLYWFDPWGQGTLVTVSSGGDGSGGGGSGGGG | |
| | | SSSELTQDPAVSVALGQTIRITCQGDSLGNYYATWYQQKPGQAPVLV | |
| | | IYGTNNRPSGIPDRFSASSSGNTASLTITGAQAEDEADYYCNSRDSS | |
| | | GHHLLFGTGTKVTVL | |
| BCMA-24 | VH | EVQLVESGGGLVQPGGSLRLSCAASGFTFSSYAMSWVRQAPGKGL | 400 |
| | | EWVSAISGSGGSTYYADSVKGRFTISRDNSKNTLYLQMNSLRAEDTA | |
| | | VYYCAKVEGSGSLDYWGQGTLVTVSS | |
| | VL | EIVMTQSPGTLSLSPGERATLSCRASQSVSSAYLAWYQQKPGQPPR | 401 |
| | | LLISGASTRATGIPDRFGGSGSGTDFTLTISRLEPEDFAVYYCQHYGS | |
| | | SFNGSSLFTFGQGTRLEIK | |
| | scFv | EVQLVESGGGLVQPGGSLRLSCAASGFTFSSYAMSWVRQAPGKGL | 402 |
| | | EWVSAISGSGGSTYYADSVKGRFTISRDNSKNTLYLQMNSLRAEDTA | |
| | | VYYCAKVEGSGSLDYWGQGTLVTVSSGGGGSGGGGSGGGGSEIV | |
| | | MTQSPGTLSLSPGERATLSCRASQSVSSAYLAWYQQKPGQPPRLLI | |
| | | SGASTRATGIPDRFGGSGSGTDFTLTISRLEPEDFAVYYCQHYGSSF | |
| | | NGSSLFTFGQGTRLEIK | |
| | VH | EVQLVETGGGLVQPGGSLRLSCAASGITFSRYPMSWVRQAPGKGLE | 403 |
| | | WVSGISDSGVSTYYADSAKGRFTISRDNSKNTLFLQMSSLRDEDTAV | |
| | | YYCVTRAGSEASDIWGQGTMVTVSS | |
| BCMA-25 | VL | EIVLTQSPATLSLSPGERATLSCRASQSVSNSLAWYQQKPGQAPRLLI | 404 |
| | | YDASSRATGIPDRFSGSGSGTDFTLTISRLEPEDFAIYYCQQFGTSSG | |
| | | LTFGGGTKLEIK | |
| | scFv | EVQLVETGGGLVQPGGSLRLSCAASGITFSRYPMSWVRQAPGKGLE | 405 |
| | | WVSGISDSGVSTYYADSAKGRFTISRDNSKNTLFLQMSSLRDEDTAV | |
| | | YYCVTRAGSEASDIWGQGTMVTVSSGGGGSGGGGSGGGGSEIVLT | |
| | | QSPATLSLSPGERATLSCRASQSVSNSLAWYQQKPGQAPRLLIYDAS | |
| | | SRATGIPDRFSGSGSGTDFTLTISRLEPEDFAIYYCQQFGTSSGLTFG | |
| | | GGTKLEIK | |
| BCMA-26 | VH | QVQLVESGGGLVQPGGSLRLSCAASGFTFSSYAMSWVRQAPGKGL | 406 |
| | | EWVSAISGSGGSTYYADSVKGRFTISRDNAKNSLYLQMNSLRAEDTA | |
| | | IYYCARATYKRELRYYYGMDVWGQGTMVTVSS | |
| | VL | EIVMTQSPGTVSLSPGERATLSCRASQSVSSSFLAWYQQKPGQAPR | 407 |
| | | LLIYGASSRATGIPDRFSGSGSGTDFTLTISRLEPEDSAVYYCQQYHS | |
| | | SPSWTFGQGTRLEIK | |
| | scFv | QVQLVESGGGLVQPGGSLRLSCAASGFTFSSYAMSWVRQAPGKGL | 408 |
| | | EWVSAISGSGGSTYYADSVKGRFTISRDNAKNSLYLQMNSLRAEDTA | |
| | | IYYCARATYKRELRYYYGMDVWGQGTMVTVSSGGGGSGGGGSGG | |
| | | GGSEIVMTQSPGTVSLSPGERATLSCRASQSVSSSFLAWYQQKPGQ | |
| | | APRLLIYGASSRATGIPDRFSGSGSGTDFTLTISRLEPEDSAVYYCQQ | |
| | | YHSSPSWTFGQGTRLEIK | |
| BCMA-27 | VH | EVQLVETGGGLVQPGGSLRLSCAASGFTFSSYAMSWVRQAPGKGL | 409 |
| | | EWVSAISGSGGSTYYADSVKGRFTISRDNSKNTLYLQMNTLKAEDTA | |
| | | VYYCARATYKRELRYYYGMDVWGQGTTVTVSS | |
| | VL | EIVLTQSPSTLSLSPGESATLSCRASQSVSTTFLAWYQQKPGQAPRL | 410 |
| | | LIYGSSNRATGIPDRFSGSGSGTDFTLTIRRLEPEDFAVYYCQQYHSS | |
| | | PSWTFGQGTKVEIK | |
| | scFv | EVQLVETGGGLVQPGGSLRLSCAASGFTFSSYAMSWVRQAPGKGL | 411 |
| | | EWVSAISGSGGSTYYADSVKGRFTISRDNSKNTLYLQMNTLKAEDTA | |
| | | VYYCARATYKRELRYYYGMDVWGQGTTVTVSSGGGGSGGGGSGG | |
| | | GGSEIVLTQSPSTLSLSPGESATLSCRASQSVSTTFLAWYQQKPGQA | |
| | | PRLLIYGSSNRATGIPDRFSGSGSGTDFTLTIRRLEPEDFAVYYCQQY | |
| | | HSSPSWTFGQGTKVEIK | |
| BCMA-28 | VH | EVQLVETGGGLVQPGRSLRLSCAASGFTFDDYAMHWVRQAPGKGL | 412 |
| | | EWVSGISWNSGSIGYADSVKGRFTISRDNAKNSLYLQMNSLRDEDTA | |
| | | VYYCARVGKAVPDVWGQGTTVTVSS | |
| | VL | DIVMTQTPSSLSASVGDRVTITCRASQSISSYLNWYQQKPGKAPKLLI | 413 |
| | | YAASSLQSGVPSRFSGSGSGTDFTLTISSLQPEDFATYYCQQSYSTP | |
| | | YSFGQGTRLEIK | |
| | scFv | EVQLVETGGGLVQPGRSLRLSCAASGFTFDDYAMHWVRQAPGKGL | 414 |
| | | EWVSGISWNSGSIGYADSVKGRFTISRDNAKNSLYLQMNSLRDEDTA | |
| | | VYYCARVGKAVPDVWGQGTTVTVSSGGGGSGGGGSGGGGSDIVM | |
| | | TQTPSSLSASVGDRVTITCRASQSISSYLNWYQQKPGKAPKLLIYAAS | |
| | | SLQSGVPSRFSGSGSGTDFTLTISSLQPEDFATYYCQQSYSTPYSFG | |
| | | QGTRLEIK | |
| BCMA-29 | VH | EVQLVESGGGLVQPGRSLRLSCTASGFTFDDYAMHWVRQRPGKGL | 415 |
| | | EWVASINWKGNSLAYGDSVKGRFAISRDNAKNTVFLQMNSLRTEDT | |
| | | AVYYCASHQGVAYYNYAMDVWGRGTLVTVSS | |
| | VL | EIVLTQSPGTLSLSPGERATLSCRATQSIGSSFLAWYQQRPGQAPRL | 416 |
| | | LIYGASQRATGIPDRFSGRGSGTDFTLTISRVEPEDSAVYYCQHYESS | |
| | | PSWTFGQGTKVEIK | |
| | scFv | EVQLVESGGGLVQPGRSLRLSCTASGFTFDDYAMHWVRQRPGKGL | 417 |
| | | EWVASINWKGNSLAYGDSVKGRFAISRDNAKNTVFLQMNSLRTEDT | |
| | | AVYYCASHQGVAYYNYAMDVWGRGTLVTVSSGGGGSGGGGSGGG | |
| | | GSEIVLTQSPGTLSLSPGERATLSCRATQSIGSSFLAWYQQRPGQAP | |
| | | RLLIYGASQRATGIPDRFSGRGSGTDFTLTISRVEPEDSAVYYCQHYE | |
| | | SSPSWTFGQGTKVEIK | |
| BCMA-30 | VH | EVQLVESGGGLVQPGGSLRLSCAASGFTFSSYAMSWVRQAPGKGL | 418 |
| | | EWVSAISGSGGSTYYADSVKGRFTISRDNSKNTLYLQMNSLRAEDTA | |
| | | VYYCAKVVRDGMDVWGQGTTVTVSS | |
| | VL | EIVLTQSPATLSLSPGERATLSCRASQSVSSSYLAWYQQKPGQAPRL | 419 |
| | | LIYGASSRATGIPDRFSGNGSGTDFTLTISRLEPEDFAVYYCQQYGSP | |
| | | PRFTFGPGTKVDIK | |
| | scFv | EVQLVESGGGLVQPGGSLRLSCAASGFTFSSYAMSWVRQAPGKGL | 420 |
| | | EWVSAISGSGGSTYYADSVKGRFTISRDNSKNTLYLQMNSLRAEDTA | |
| | | VYYCAKVVRDGMDVWGQGTTVTVSSGGGGSGGGGSGGGGSEIVL | |
| | | TQSPATLSLSPGERATLSCRASQSVSSSYLAWYQQKPGQAPRLLIYG | |
| | | ASSRATGIPDRFSGNGSGTDFTLTISRLEPEDFAVYYCQQYGSPPRF | |
| | | TFGPGTKVDIK | |
| BCMA-31 | VH | EVQLLESGGGLVQPGGSLRLSCAASGFTFSSYAMSWVRQAPGKGL | 421 |
| | | EWVSAISGSGGSTYYADSVKGRFTISRDNSKNTLYLQMNSLRAEDTA | |
| | | VYYCAKIPQTGTFDYWGQGTLVTVSS | |
| | VL | EIVLTQSPGTLSLSPGERATLSCRASQSVSSSYLAWYQQRPGQAPRL | 422 |
| | | LIYGASSRATGIPDRFSGSGSGTDFTLTISRLEPEDFAVYYCQHYGSS | |
| | | PSWTFGQGTRLEIK | |
| | scFv | EVQLLESGGGLVQPGGSLRLSCAASGFTFSSYAMSWVRQAPGKGL | 423 |
| | | EWVSAISGSGGSTYYADSVKGRFTISRDNSKNTLYLQMNSLRAEDTA | |
| | | VYYCAKIPQTGTFDYWGQGTLVTVSSGGGGSGGGGSGGGGSEIVLT | |
| | | QSPGTLSLSPGERATLSCRASQSVSSSYLAWYQQRPGQAPRLLIYG | |
| | | ASSRATGIPDRFSGSGSGTDFTLTISRLEPEDFAVYYCQHYGSSPSW | |
| | | TFGQGTRLEIK | |
| BCMA-32 | VH | EVQLVETGGGLVQPGGSLRLSCAASGFTFSSYAMSWVRQAPGKGL | 424 |
| | | EWVSAISGSGGSTYYADSVKGRFTMSRENDKNSVFLQMNSLRVEDT | |
| | | GVYYCARANYKRELRYYYGMDVWGQGTMVTVSS | |
| | VL | EIVMTQSPGTLSLSPGESATLSCRASQRVASNYLAWYQHKPGQAPS | 425 |
| | | LLISGASSRATGVPDRFSGSGSGTDFTLAISRLEPEDSAVYYCQHYD | |
| | | SSPSWTFGQGTKVEIK | |
| | scFv | EVQLVETGGGLVQPGGSLRLSCAASGFTFSSYAMSWVRQAPGKGL | 426 |
| | | EWVSAISGSGGSTYYADSVKGRFTMSRENDKNSVFLQMNSLRVEDT | |
| | | GVYYCARANYKRELRYYYGMDVWGQGTMVTVSSGGGGSGGGGSG | |
| | | GGGSEIVMTQSPGTLSLSPGESATLSCRASQRVASNYLAWYQHKPG | |
| | | QAPSLLISGASSRATGVPDRFSGSGSGTDFTLAISRLEPEDSAVYYC | |
| | | QHYDSSPSWTFGQGTKVEIK | |
| BCMA-33 | VH | EVQLLETGGGLVQPGGSLRLSCAASGFSFSSYAMSWVRQAPGKGL | 427 |
| | | EWVSAISGSGGSTYYADSVKGRFTISRDNSKNTLYLQMNSLRAEDTA | |
| | | VYYCAKALVGATGAFDIWGQGTLVTVSS | |
| | VL | EIVLTQSPGTLSLSPGERATLSCRASQSLSSNFLAWYQQKPGQAPGL | 428 |
| | | LIYGASNWATGTPDRFSGSGSGTDFTLTITRLEPEDFAVYYCQYYGT | |
| | | SPMYTFGQGTKVEIK | |
| | scFv | EVQLLETGGGLVQPGGSLRLSCAASGFSFSSYAMSWVRQAPGKGL | 429 |
| | | EWVSAISGSGGSTYYADSVKGRFTISRDNSKNTLYLQMNSLRAEDTA | |
| | | VYYCAKALVGATGAFDIWGQGTLVTVSSGGGGSGGGGSGGGGSEI | |
| | | VLTQSPGTLSLSPGERATLSCRASQSLSSNFLAWYQQKPGQAPGLLI | |
| | | YGASNWATGTPDRFSGSGSGTDFTLTITRLEPEDFAVYYCQYYGTSP | |
| | | MYTFGQGTKVEIK | |
| BCMA-34 | VH | EVQLLESGGGLVQPGGSLRLSCAASGFTFSSYAMSWVRQAPGKGL | 430 |
| | | EWVSAISGSGGSTYYADSVKGRFTISRDNSKNTLYLQMNSLRAEDTA | |
| | | VYYCVLWFGEGFDPWGQGTLVTVSS | |
| | VL | DIVLTQSPLSLPVTPGEPASISCRSSQSLLHSNGYNYLDWYLQKPGQ | 431 |
| | | SPQLLIYLGSNRASGVPDRFSGSGSGTDFTLKISRVEAEDVGVYYCM | |
| | | QALQTPLTFGGGTKVDIK | |
| | scFv | EVQLLESGGGLVQPGGSLRLSCAASGFTFSSYAMSWVRQAPGKGL | 432 |
| | | EWVSAISGSGGSTYYADSVKGRFTISRDNSKNTLYLQMNSLRAEDTA | |
| | | VYYCVLWFGEGFDPWGQGTLVTVSSGGGGSGGGGSGGGGSDIVLT | |
| | | QSPLSLPVTPGEPASISCRSSQSLLHSNGYNYLDWYLQKPGQSPQLL | |
| | | IYLGSNRASGVPDRFSGSGSGTDFTLKISRVEAEDVGVYYCMQALQT | |
| | | PLTFGGGTKVDIK | |
| BCMA-35 | VH | QVQLVESGGGLVQPGGSLRLSCAASGFTFSSYAMSWVRQAPGKGL | 433 |
| | | EWVSAISGSGGSTYYADSVKGRFTISRDNSKNTLYLQMNSLRAEDTA | |
| | | VYYCAKVGYDSSGYYRDYYGMDVWGQGTTVTVSS | |
| | VL | EIVLTQSPGTLSLSPGERATLSCRASQSVSSSYLAWYQQKPGQAPRL | 434 |
| | | LIYGTSSRATGISDRFSGSGSGTDFTLTISRLEPEDFAVYYCQHYGNS | |
| | | PPKFTFGPGTKLEIK | |
| | scFv | QVQLVESGGGLVQPGGSLRLSCAASGFTFSSYAMSWVRQAPGKGL | 435 |
| | | EWVSAISGSGGSTYYADSVKGRFTISRDNSKNTLYLQMNSLRAEDTA | |
| | | VYYCAKVGYDSSGYYRDYYGMDVWGQGTTVTVSSGGGGSGGGGS | |
| | | GGGGSEIVLTQSPGTLSLSPGERATLSCRASQSVSSSYLAWYQQKP | |
| | | GQAPRLLIYGTSSRATGISDRFSGSGSGTDFTLTISRLEPEDFAVYYC | |
| | | QHYGNSPPKFTFGPGTKLEIK | |
| BCMA-36 | VH | EVQLVESGGGLVQPGGSLRLSCAASGFTFSSYAMSWVRQAPGKGL | 436 |
| | | EWVSAISGSGGSTYYADSVKGRFTISRDNSKNTLYLQMNSLRAEDTA | |
| | | VYYCAKMGWSSGYLGAFDIWGQGTTVTVSS | |
| | VL | EIVLTQSPGTLSLSPGERATLSCRASQSVASSFLAWYQQKPGQAPRL | 437 |
| | | LIYGASGRATGIPDRFSGSGSGTDFTLTISRLEPEDFAVYYCQHYGGS | |
| | | PRLTFGGGTKVDIK | |
| | scFv | EVQLVESGGGLVQPGGSLRLSCAASGFTFSSYAMSWVRQAPGKGL | 438 |
| | | EWVSAISGSGGSTYYADSVKGRFTISRDNSKNTLYLQMNSLRAEDTA | |
| | | VYYCAKMGWSSGYLGAFDIWGQGTTVTVSSGGGGSGGGGSGGGG | |
| | | SEIVLTQSPGTLSLSPGERATLSCRASQSVASSFLAWYQQKPGQAPR | |
| | | LLIYGASGRATGIPDRFSGSGSGTDFTLTISRLEPEDFAVYYCQHYGG | |
| | | SPRLTFGGGTKVDIK | |
| BCMA-37 | VH | QIQLVQSGPDLKKPGETVKLSCKASGYTFTNFGMNWVKQAPGKGFK | 439 |
| | | WMAWINTYTGESYFADDFKGRFAFSVETSATTAYLQINNLKTEDTAT | |
| | | YFCARGEIYYGYDGGFAYWGQGTLVTVSA | |
| | VL | DVVMTQSHRFMSTSVGDRVSITCRASQDVNTAVSWYQQKPGQSPK | 440 |
| | | LLIFSASYRYTGVPDRFTGSGSGADFTLTISSVQAEDLAVYYCQQHYS | |
| | | TPWTFGGGTKLDIK | |
| | scFv | QIQLVQSGPDLKKPGETVKLSCKASGYTFTNFGMNWVKQAPGKGFK | 441 |
| | | WMAWINTYTGESYFADDFKGRFAFSVETSATTAYLQINNLKTEDTAT | |
| | | YFCARGEIYYGYDGGFAYWGQGTLVTVSAGGGGSGGGGSGGGGS | |
| | | DVVMTQSHRFMSTSVGDRVSITCRASQDVNTAVSWYQQKPGQSPK | |
| | | LLIFSASYRYTGVPDRFTGSGSGADFTLTISSVQAEDLAVYYCQQHYS | |
| | | TPWTFGGGTKLDIK | |
| BCMA-38 | VH | QIQLVQSGPELKKPGETVKISCKASGYTFTDYSINWVKRAPGKGLKW | 442 |
| | | MGWINTETREPAYAYDFRGRFAFSLETSASTAYLQINNLKYEDTATYF | |
| | | CALDYSYAMDYWGQGTSVTVSS | |
| | VL | DIVLTQSPASLAMSLGKRATISCRASESVSVIGAHLIHWYQQKPGQPP | 443 |
| | | KLLIYLASNLETGVPARFSGSGSGTDFTLTIDPVEEDDVAIYSCLQSRI | |
| | | FPRTFGGGTKLEIK | |
| | scFv | QIQLVQSGPELKKPGETVKISCKASGYTFTDYSINWVKRAPGKGLKW | 444 |
| | | MGWINTETREPAYAYDFRGRFAFSLETSASTAYLQINNLKYEDTATYF | |
| | | CALDYSYAMDYWGQGTSVTVSSGGGGSGGGGSGGGGSQIQLVQS | |
| | | GPELKKPGETVKISCKASGYTFTDYSINWVKRAPGKGLKWMGWINTE | |
| | | TREPAYAYDFRGRFAFSLETSASTAYLQINNLKYEDTATYFCALDYSY | |
| | | AMDYWGQGTSVTVSS | |
| BCMA-39 | VH | QIQLVQSGPELKKPGETVKISCKASGYTFRHYSMNWVKQAPGKGLK | 445 |
| | | WMGRINTESGVPIYADDFKGRFAFSVETSASTAYLVINNLKDEDTASY | |
| | | FCSNDYLYSLDFWGQGTALTVSS | |
| | VL | DIVLTQSPPSLAMSLGKRATISCRASESVTILGSHLIYWYQQKPGQPP | 446 |
| | | TLLIQLASNVQTGVPARFSGSGSRTDFTLTIDPVEEDDVAVYYCLQSR | |
| | | TIPRTFGGGTKLEIK | |
| | scFv | QIQLVQSGPELKKPGETVKISCKASGYTFRHYSMNWVKQAPGKGLK | 447 |
| | | WMGRINTESGVPIYADDFKGRFAFSVETSASTAYLVINNLKDEDTASY | |
| | | FCSNDYLYSLDFWGQGTALTVSSGGGGSGGGGSGGGGSDIVLTQS | |
| | | PPSLAMSLGKRATISCRASESVTILGSHLIYWYQQKPGQPPTLLIQLAS | |
| | | NVQTGVPARFSGSGSRTDFTLTIDPVEEDDVAVYYCLQSRTIPRTFG | |
| | | GGTKLEIK | |
| BCMA-40 | VH | QIQLVQSGPELKKPGETVKISCKASGYTFTHYSMNWVKQAPGKGLK | 448 |
| | | WMGRINTETGEPLYADDFKGRFAFSLETSASTAYLVINNLKNEDTATF | |
| | | FCSNDYLYSCDYWGQGTTLTVSS | |
| | VL | DIVLTQSPPSLAMSLGKRATISCRASESVTILGSHLIYWYQQKPGQPP | 446 |
| | | TLLIQLASNVQTGVPARFSGSGSRTDFTLTIDPVEEDDVAVYYCLQSR | |
| | | TIPRTFGGGTKLEIK | |
| | scFv | QIQLVQSGPELKKPGETVKISCKASGYTFTHYSMNWVKQAPGKGLK | 449 |
| | | WMGRINTETGEPLYADDFKGRFAFSLETSASTAYLVINNLKNEDTATF | |
| | | FCSNDYLYSCDYWGQGTTLTVSSGGGGSGGGGSGGGGSDIVLTQS | |
| | | PPSLAMSLGKRATISCRASESVTILGSHLIYWYQQKPGQPPTLLIQLAS | |
| | | NVQTGVPARFSGSGSRTDFTLTIDPVEEDDVAVYYCLQSRTIPRTFG | |
| | | GGTKLEIK | |

TABLE 14B
BCMA Binders-Light chain CDRsequences
according to Kabatnumbering scheme

| | | SEQ | | SEQ | | SEQ |
|---|---|---|---|---|---|---|
| | | ID | | ID | | ID |
| Binder | CDR-L1 | NO: | CDR-L2 | NO: | CDR-L3 | NO: |
| BCMA-1 | RASQS | 450 | AASSL | 482 | QQSYS | 511 |
| | ISSYL | | QS | | TPYT | |
| | N | | | | | |
| BCMA-2 | RASQS | 451 | GASRR | 483 | QQYHS | 512 |
| | ISSSF | | AT | | SPSWT | |
| | LA | | | | | |
| BCMA-3 | RSSQS | 452 | LGSNR | 484 | MQALQ | 513 |
| | LLHSN | | AS | | TPYT | |
| | GYNYL | | | | | |
| | D | | | | | |
| BCMA-4 | KSSQS | 453 | EVSNR | 485 | MQNIQ | 514 |
| | LLRND | | FS | | FPS | |
| | GKTPL | | | | | |
| | Y | | | | | |
| BCMA-5 | RSSQS | 454 | LGSKR | 486 | MQALQ | 513 |
| | LLHSN | | AS | | TPYT | |
| | GYNYL | | | | | |
| | N | | | | | |
| BCMA-6 | RASQS | 450 | GASTL | 487 | QQSYK | 515 |
| | ISSYL | | AS | | RAS | |
| | N | | | | | |
| BCMA-7 | RSSQS | 455 | LGSNR | 484 | MQGRQ | 516 |
| | LLYSN | | AS | | FPYS | |
| | GYNYV | | | | | |
| | D | | | | | |
| BCMA-8 | RASQS | 456 | GASTR | 488 | QQYGS | 517 |
| | VSSNL | | AS | | SLT | |
| | A | | | | | |
| BCMA-9 | RASQS | 457 | GASIR | 489 | QQYGS | 518 |
| | VSSKL | | AT | | SSWT | |
| | A | | | | | |
| BCMA- | RASQS | 458 | DASNR | 221 | QQYGS | 519 |
| 10 | VGSTN | | AT | | SPPWT | |
| | LA | | | | | |
| BCMA- | RASQS | 450 | AASSL | 482 | QQSYT | 520 |
| 11 | ISSYL | | QS | | LA | |
| | N | | | | | |
| BCMA- | KSSES | 459 | EVSNR | 490 | MQGTH | 521 |
| 12 | LVHNS | | DS | | WPGT | |
| | GKTYL | | | | | |
| | N | | | | | |
| BCMA- | QASED | 460 | DASTL | 491 | QQYES | 522 |
| 13 | INKFL | | QT | | LPLT | |
| | N | | | | | |
| BCMA- | RASQS | 461 | GASTR | 492 | QQYND | 523 |
| 14 | VGSNL | | AT | | WLPVT | |
| | A | | | | | |
| BCMA- | RASQS | 462 | GASSR | 493 | QQYAG | 524 |
| 15 | IGSSS | | AS | | SPPFT | |
| | LA | | | | | |
| BCMA- | KASQD | 463 | SATSP | 494 | LQHDN | 525 |
| 16 | IDDAM | | VP | | FPLT | |
| | N | | | | | |
| BCMA- | RASQD | 464 | AANKS | 495 | QHYYR | 526 |
| 17 | IYNNL | | QS | | FPYS | |
| | A | | | | | |
| BCMA- | RSSQS | 452 | LGSNR | 484 | MQALQ | 513 |
| 18 | LLHSN | | AS | | TPYT | |
| | GYNYL | | | | | |
| | D | | | | | |
| BCMA- | GGNNI | 465 | DDSVR | 496 | QVWDS | 527 |
| 19 | GTKSV | | PS | | DSEHV | |
| | H | | | | V | |
| BCMA- | SGDGL | 466 | RDKER | 497 | QAWDD | 528 |
| 20 | SKKYV | | PS | | TTVV | |
| | S | | | | | |
| BCMA- | RASQG | 467 | AASNL | 498 | QKYNS | 529 |
| 21 | IRNWL | | QS | | APFT | |
| | A | | | | | |
| BCMA- | GGNNI | 468 | GKNNR | 499 | SSRDS | 530 |
| 22 | GSKSV | | PS | | SGDHL | |
| | H | | | | RV | |
| BCMA- | QGDSL | 469 | GTNNR | 500 | NSRDS | 531 |
| 23 | GNYYA | | PS | | SGHHL | |
| | T | | | | L | |
| BCMA- | RASQS | 470 | GASTR | 492 | QHYGS | 532 |
| 24 | VSSAY | | AT | | SFNGS | |
| | LA | | | | SLF | |
| | | | | | T | |
| BCMA- | RASQS | 471 | DASSR | 501 | QQFGT | 533 |
| 25 | VSNSL | | AT | | SSGLT | |
| | A | | | | | |
| BCMA- | RASQS | 472 | GASSR | 502 | QQYHS | 512 |
| 26 | VSSSF | | AT | | SPSWT | |
| | LA | | | | | |
| BCMA- | RASQS | 473 | GSSNR | 503 | QQYHS | 512 |
| 27 | VSTTF | | AT | | SPSWT | |
| | LA | | | | | |
| BCMA- | RASQS | 450 | AASSL | 482 | QQSYS | 534 |
| 28 | ISSYL | | QS | | TPYS | |
| | N | | | | | |
| BCMA- | RATQS | 474 | GASQR | 504 | QHYES | 535 |
| 29 | IGSSF | | AT | | SPSWT | |
| | LA | | | | | |
| BCMA- | RASQS | 475 | GASSR | 502 | QQYGS | 536 |
| 30 | VSSSY | | AT | | PPRFT | |
| | LA | | | | | |
| BCMA- | RASQS | 475 | GASSR | 502 | QHYGS | 537 |
| 31 | VSSSY | | AT | | SPSWT | |
| | LA | | | | | |
| BCMA- | RASQR | 476 | GASSR | 502 | QHYDS | 538 |
| 32 | VASNY | | AT | | SPSWT | |
| | LA | | | | | |
| BCMA- | RASQS | 477 | GASNW | 505 | QYYGT | 539 |
| 33 | LSSNF | | AT | | SPMYT | |
| | LA | | | | | |
| BCMA- | RSSQS | 452 | LGSNR | 484 | MQALQ | 540 |
| 34 | LLHSN | | AS | | TPLT | |
| | GYNYL | | | | | |
| | D | | | | | |
| BCMA- | RASQS | 475 | GTSSR | 506 | QHYGN | 541 |
| 35 | VSSSY | | AT | | SPPKF | |
| | LA | | | | T | |
| BCMA- | RASQS | 478 | GASGR | 507 | QHYGG | 542 |
| 36 | VASSF | | AT | | SPRLT | |
| | LA | | | | | |
| BCMA- | RASQD | 479 | SASYR | 508 | QQHYS | 543 |
| 37 | VNTAV | | YT | | TPWT | |
| | S | | | | | |
| BCMA- | RASES | 480 | LASNL | 509 | LQSRI | 544 |
| 38 | VSVIG | | ET | | FPRT | |
| | AHLIH | | | | | |
| BCMA- | RASES | 481 | LASNV | 510 | LQSRT | 545 |
| 39 | VTILG | | QT | | IPRT | |
| | SHLIY | | | | | |
| BCMA- | RASES | 481 | LASNV | 510 | LQSRT | 545 |
| 40 | VTILG | | QT | | IPRT | |
| | SHLIY | | | | | |

TABLE 14C
BCMA Binders-Light chain CDR sequences
according to Chothia numbering scheme

| | | SEQ | | SEQ | | SEQ |
|---|---|---|---|---|---|---|
| | | ID | CDR- | ID | | ID |
| Binder | CDR-L1 | NO: | L2 | NO: | CDR-L3 | NO: |
| BCMA-1 | SQSISSY | 546 | AAS | 577 | SYSTPY | 590 |
| BCMA-2 | SQSISSSF | 547 | GAS | 578 | YHSSPSW | 591 |
| BCMA-3 | SQSLLHSN | 548 | LGS | 579 | ALQTPY | 592 |
| | GYNY | | | | | |
| BCMA-4 | SQSLLRND | 549 | EVS | 580 | NIQFP | 593 |
| | GKTP | | | | | |
| BCMA-5 | SQSLLHSN | 548 | LGS | 579 | ALQTPY | 592 |
| | GYNY | | | | | |
| BCMA-6 | SQSISSY | 546 | GAS | 578 | SYKRA | 594 |
| BCMA-7 | SQSLLYSN | 550 | LGS | 579 | GRQFPY | 595 |
| | GYNY | | | | | |
| BCMA-8 | SQSVSSN | 551 | GAS | 578 | YGSSL | 596 |
| BCMA-9 | SQSVSSK | 552 | GAS | 578 | YGSSSW | 597 |
| BCMA-10 | SQSVGSTN | 553 | DAS | 281 | YGSSPPW | 598 |
| BCMA-11 | SQSISSY | 546 | AAS | 577 | SYTL | 599 |
| BCMA-12 | SESLVHNS | 554 | EVS | 580 | GTHWPG | 600 |
| | GKTY | | | | | |
| BCMA-13 | SEDINKF | 555 | DAS | 281 | YESLPL | 601 |
| BCMA-14 | SQSVGSN | 556 | GAS | 578 | YNDWLPV | 602 |
| BCMA-15 | SQSIGSSS | 557 | GAS | 578 | YAGSPPF | 603 |
| BCMA-16 | SQDIDDA | 558 | SAT | 581 | HDNFPL | 604 |
| BCMA-17 | SQDIYNN | 559 | AAN | 582 | YYRFPY | 605 |
| BCMA-18 | SQSLLHS | 548 | LGS | 579 | ALQTPY | 592 |
| | NGYNY | | | | | |
| BCMA-19 | NNIGTKS | 560 | DDS | 583 | WDSDSEHV | 606 |
| BCMA-20 | DGLSKKY | 561 | RDK | 584 | WDDTTV | 607 |
| BCMA-21 | SQGIRNW | 562 | AAS | 577 | YNSAPF | 608 |
| BCMA-22 | NNIGSKS | 563 | GKN | 585 | RDSSGDH | 609 |
| | | | | | LR | |
| BCMA-23 | DSLGNYY | 564 | GTN | 277 | RDSSGHHL | 610 |
| BCMA-24 | SQSVSSAY | 565 | GAS | 578 | YGSSFNGS | 611 |
| | | | | | SLF | |
| BCMA-25 | SQSVSNS | 566 | DAS | 281 | FGTSSGL | 612 |
| BCMA-26 | SQSVSSSF | 567 | GAS | 578 | YHSSPSW | 591 |
| BCMA-27 | SQSVSTTF | 568 | GSS | 586 | YHSSPSW | 591 |
| BCMA-28 | SQSISSY | 546 | AAS | 577 | SYSTPY | 590 |
| BCMA-29 | TQSIGSSF | 569 | GAS | 578 | YESSPSW | 613 |
| BCMA-30 | SQSVSSSY | 570 | GAS | 578 | YGSPPRF | 614 |
| BCMA-31 | SQSVSSSY | 570 | GAS | 578 | YGSSPSW | 615 |
| BCMA-32 | SQRVASNY | 571 | GAS | 578 | YDSSPSW | 616 |
| BCMA-33 | SQSLSSNF | 572 | GAS | 578 | YGTSPMY | 617 |
| BCMA-34 | SQSLLHSN | 548 | LGS | 579 | ALQTPL | 618 |
| | GYNY | | | | | |
| BCMA-35 | SQSVSSSY | 570 | GTS | 587 | YGNSPPKF | 619 |
| BCMA-36 | SQSVASSF | 573 | GAS | 578 | YGGSPRL | 620 |
| BCMA-37 | SQDVNTA | 574 | SAS | 588 | HYSTPW | 621 |
| BCMA-38 | SESVSVI | 575 | LAS | 589 | SRIFPR | 622 |
| | GAHL | | | | | |
| BCMA-39 | SESVTIL | 576 | LAS | 589 | SRTIPR | 623 |
| | GSHL | | | | | |
| BCMA-40 | SESVTIL | 576 | LAS | 589 | SRTIPR | 623 |
| | GSHL | | | | | |

TABLE 14D
BCMA Binders-Light chain CDR sequences
according to combination of Kabat and
Chothia numbering schemes

| | | SEQ | | SEQ | | SEQ |
|---|---|---|---|---|---|---|
| | | ID | | ID | | ID |
| Binder | CDR-L1 | NO: | CDR-L2 | NO: | CDR-L3 | NO: |
| BCMA-1 | RASQSIS | 450 | AASSLQS | 482 | QQSYSTP | 511 |
| | SYLN | | | | YT | |
| BCMA-2 | RASQSIS | 451 | GASRRAT | 483 | QQYHSSP | 512 |
| | SSFLA | | | | SW | |
| | | | | | T | |
| BCMA-3 | RSSQSLL | 452 | LGSNRAS | 484 | MQALQTP | 513 |
| | HSNGY | | | | YT | |
| | NYLD | | | | | |
| BCMA-4 | KSSQSLL | 453 | EVSNRFS | 485 | MQNIQFP | 514 |
| | RNDGKT | | | | S | |
| | PLY | | | | | |
| BCMA-5 | RSSQSLL | 454 | LGSKRAS | 486 | MQALQTP | 513 |
| | HSNGY | | | | YT | |
| | NYLN | | | | | |
| BCMA-6 | RASQSIS | 450 | GASTLAS | 487 | QQSYKRA | 515 |
| | SYLN | | | | S | |
| BCMA-7 | RSSQSLL | 455 | LGSNRAS | 484 | MQGRQFP | 516 |
| | YSNGY | | | | YS | |
| | NYVD | | | | | |
| BCMA-8 | RASQSVS | 456 | GASTRAS | 488 | QQYGSSL | 517 |
| | SNLA | | | | T | |
| BCMA-9 | RASQSVS | 457 | GASIRAT | 489 | QQYGSSS | 518 |
| | SKLA | | | | WT | |
| BCMA-10 | RASQSVG | 458 | DASNRAT | 221 | QQYGSSP | 519 |
| | STNLA | | | | PW | |
| | | | | | T | |
| BCMA-11 | RASQSIS | 450 | AASSLQS | 482 | QQSYTLA | 520 |
| | SYLN | | | | | |
| BCMA-12 | KSSESLV | 459 | EVSNRDS | 490 | MQGTHWP | 521 |
| | HNSGKT | | | | GT | |
| | YLN | | | | | |
| BCMA-13 | QASEDIN | 460 | DASTLQT | 491 | QQYESLP | 522 |
| | KFLN | | | | LT | |
| BCMA-14 | RASQSVG | 461 | GASTRAT | 492 | QQYNDWL | 523 |
| | SNLA | | | | PV | |
| | | | | | T | |
| BCMA-15 | RASQSIG | 462 | GASSRAS | 493 | QQYAGSP | 524 |
| | SSSLA | | | | PFT | |
| BCMA-16 | KASQDID | 463 | SATSPVP | 494 | LQHDNFP | 525 |
| | DAMN | | | | LT | |
| BCMA-17 | RASQDIY | 464 | AANKSQS | 495 | QHYYRFP | 526 |
| | NNLA | | | | YS | |
| BCMA-18 | RSSQSLL | 452 | LGSNRAS | 484 | MQALQTP | 513 |
| | HSNGY | | | | YT | |
| | NYLD | | | | | |
| BCMA-19 | GGNNIGT | 465 | DDSVRPS | 496 | QVWDSDS | 527 |
| | KSVH | | | | EH | |
| | | | | | W | |
| BCMA-20 | SGDGLSK | 466 | RDKERPS | 497 | QAWDDTT | 528 |
| | KYVS | | | | VV | |
| BCMA-21 | RASQGIR | 467 | AASNLQS | 498 | QKYNSAP | 529 |
| | NWLA | | | | FT | |
| BCMA-22 | GGNNIGS | 468 | GKNNRPS | 499 | SSRDSSG | 530 |
| | KSVH | | | | DHL | |
| | | | | | RV | |
| BCMA-23 | QGDSLGN | 469 | GTNNRPS | 500 | NSRDSSG | 531 |
| | YYAT | | | | HHL | |
| | | | | | L | |
| BCMA-24 | RASQSVS | 470 | GASTRAT | 492 | QHYGSSF | 532 |
| | SAYLA | | | | NG | |
| | | | | | SSLFT | |
| BCMA-25 | RASQSVS | 471 | DASSRAT | 501 | QQFGTSS | 533 |
| | NSLA | | | | GLT | |
| BCMA-26 | RASQSVS | 472 | GASSRAT | 502 | QQYHSSP | 512 |
| | SSFLA | | | | SW | |
| | | | | | T | |
| BCMA-27 | RASQSVS | 473 | GSSNRAT | 503 | QQYHSSP | 512 |
| | TTFLA | | | | SW | |
| | | | | | T | |
| BCMA-28 | RASQSIS | 450 | AASSLQS | 482 | QQSYSTP | 534 |
| | SYLN | | | | YS | |
| BCMA-29 | RATQSIG | 474 | GASQRAT | 504 | QHYESSP | 535 |
| | SSFLA | | | | SW | |
| | | | | | T | |
| BCMA-30 | RASQSVS | 475 | GASSRAT | 502 | QQYGSPP | 536 |
| | SSYLA | | | | RFT | |
| BCMA-31 | RASQSVS | 475 | GASSRAT | 502 | QHYGSSP | 537 |
| | SSYLA | | | | SW | |
| | | | | | T | |
| BCMA-32 | RASQRVA | 476 | GASSRAT | 502 | QHYDSSP | 538 |
| | SNYLA | | | | SW | |
| | | | | | T | |
| BCMA-33 | RASQSLS | 477 | GASNWAT | 505 | QYYGTSP | 539 |
| | SNFLA | | | | MYT | |
| BCMA-34 | RSSQSLL | 452 | LGSNRAS | 484 | MQALQTP | 540 |
| | HSNGY | | | | LT | |
| | NYLD | | | | | |
| BCMA-35 | RASQSVS | 475 | GTSSRAT | 506 | QHYGNSP | 541 |
| | SSYLA | | | | PKF | |
| | | | | | T | |
| BCMA-36 | RASQSVA | 478 | GASGRAT | 507 | QHYGGSP | 542 |
| | SSFLA | | | | RLT | |
| BCMA-37 | RASQDVN | 479 | SASYRYT | 508 | QQHYSTP | 543 |
| | TAVS | | | | WT | |
| BCMA-38 | RASESVS | 480 | LASNLET | 509 | LQSRIFP | 544 |
| | VIGAHLI | | | | RT | |
| | H | | | | | |
| BCMA-39 | RASESVT | 481 | LASNVQT | 510 | LQSRTIP | 545 |
| | ILGSHLI | | | | RT | |
| | Y | | | | | |
| BCMA-40 | RASESVT | 481 | LASNVQT | 510 | LQSRTIP | 545 |
| | ILGSHLI | | | | RT | |
| | Y | | | | | |

TABLE 14E
BCMA Binders-Heavy chain CDR sequences
according to Kabat numbering scheme

| | | SEQ | | SEQ | | SEQ |
|---|---|---|---|---|---|---|
| | CDR- | ID | | ID | | ID |
| Antibody | H1 | NO: | CDR-H2 | NO: | CDR-H3 | NO: |
| BCMA-1 | NHGMS | 624 | GIVYSGS | 639 | HGGESDV | 656 |
| | | | TYYAASV | | | |
| | | | KG | | | |
| BCMA-2 | NYAMS | 1073 | GISRSGE | 750 | SPAHYYG | 722 |
| | | | NTYYADS | | GMDV | |
| | | | VKG | | | |
| BCMA-3 | DYAMH | 635 | GISWNSG | 650 | HSFLAY | 723 |
| | | | SIGYADS | | | |
| | | | VKG | | | |
| BCMA-4 | NHGMS | 624 | GIVYSGS | 639 | HGGESDV | 656 |
| | | | TYYAASV | | | |
| | | | KG | | | |
| BCMA-5 | NFGIN | 1074 | WINPKNN | 751 | GPYYYQS | 724 |
| | | | NTNYAQ | | YMDV | |
| | | | KFQG | | | |
| BCMA-6 | SDAMT | 1075 | VISGSGG | 752 | LDSSGYY | 725 |
| | | | TTYYADS | | YARGPRY | |
| | | | VKG | | | |
| BCMA-7 | NYGIT | 1076 | WISAYNG | 753 | GPYYYYM | 726 |
| | | | NTNYAQ | | DV | |
| | | | KFQG | | | |
| BCMA-8 | NHGMS | 624 | GIVYSGS | 639 | HGGESDV | 656 |
| | | | TYYAASV | | | |
| | | | KG | | | |
| BCMA-9 | NHGMS | 624 | GIVYSGS | 639 | HGGESDV | 656 |
| | | | TYYAASV | | | |
| | | | KG | | | |
| BCMA-10 | NHGMS | 624 | GIVYSGS | 639 | HGGESDV | 656 |
| | | | TYYAASV | | | |
| | | | KG | | | |
| BCMA-11 | DYYMS | 628 | YISSSGS | 643 | ESGDGMD | 727 |
| | | | TIYYADS | | V | |
| | | | VKG | | | |
| BCMA-12 | DYYMS | 628 | YISSSGN | 754 | STMVRED | 728 |
| | | | TIYYADS | | Y | |
| | | | VKG | | | |
| BCMA-13 | NHGMS | 624 | GIVYSGS | 639 | HGGESDV | 656 |
| | | | TYYAASV | | | |
| | | | KG | | | |
| BCMA-14 | NHGMS | 624 | GIVYSGS | 639 | HGGESDV | 656 |
| | | | TYYAASV | | | |
| | | | KG | | | |
| BCMA-15 | NHGMS | 624 | GIVYSGS | 639 | HGGESDV | 656 |
| | | | TYYAASV | | | |
| | | | KG | | | |
| BCMA-16 | SSYYY | 625 | SIYYSGS | 640 | HWQEWPD | 657 |
| | WG | | AYYNPSL | | AFDI | |
| | | | KS | | | |
| BCMA-17 | TSGMC | 626 | RIDWDED | 641 | SGAGGTS | 658 |
| | VS | | KFYSTSL | | ATAFDI | |
| | | | KT | | | |
| BCMA-18 | SYSMN | 627 | SISSSSS | 642 | TIAAVYA | 659 |
| | | | YIYYADS | | FDI | |
| | | | V | | | |
| | | | KG | | | |
| BCMA-19 | DYYMS | 628 | YISSSGS | 643 | DLRGAFD | 660 |
| | | | TIYYADS | | I | |
| | | | VKG | | | |
| BCMA-20 | SHYIH | 629 | MINPSGG | 644 | EGSGSGW | 661 |
| | | | VTAYSQ | | YFDF | |
| | | | TLQG | | | |
| BCMA-21 | SGGYY | 630 | YIYYSGS | 645 | AGIAARL | 662 |
| | WS | | TYYNPSL | | RGAFDI | |
| | | | KS | | | |
| BCMA-22 | SYAIS | 631 | GIIPIFG | 646 | RGGYQLL | 663 |
| | | | TANYAQK | | RWDVGLL | |
| | | | F | | R | |
| | | | QG | | SAFDI | |
| BCMA-23 | SNSAA | 632 | RTYYRSK | 647 | SSPEGLF | 664 |
| | WN | | WYSFYAI | | LYWFDP | |
| | | | SLKS | | | |
| BCMA-24 | SYAMS | 633 | AISGSGG | 648 | VEGSGSL | 665 |
| | | | STYYADS | | DY | |
| | | | VKG | | | |
| BCMA-25 | RYPMS | 634 | GISDSGV | 649 | RAGSEAS | 666 |
| | | | STYYADS | | DI | |
| | | | AKG | | | |
| BCMA-26 | SYAMS | 633 | AISGSGG | 648 | ATYKREL | 667 |
| | | | STYYADS | | RYYYGMD | |
| | | | | | V | |
| | | | VKG | | | |
| BCMA-27 | SYAMS | 633 | AISGSGG | 648 | ATYKREL | 667 |
| | | | STYYADS | | RYYYGMD | |
| | | | | | V | |
| | | | VKG | | | |
| BCMA-28 | DYAMH | 635 | GISWNSG | 650 | VGKAVPD | 668 |
| | | | SIGYADS | | V | |
| | | | VKG | | | |
| BCMA-29 | DYAMH | 635 | SINWKGN | 651 | HQGVAYY | 669 |
| | | | SLAYGD | | NYAMDV | |
| | | | SVKG | | | |
| BCMA-30 | SYAMS | 633 | AISGSGG | 648 | VVRDGMD | 670 |
| | | | STYYADS | | V | |
| | | | VKG | | | |
| BCMA-31 | SYAMS | 633 | AISGSGG | 648 | IPQTGTF | 671 |
| | | | STYYADS | | DY | |
| | | | VKG | | | |
| BCMA-32 | SYAMS | 633 | AISGSGG | 648 | ANYKREL | 672 |
| | | | STYYADS | | RYYYGMD | |
| | | | | | V | |
| | | | VKG | | | |
| BCMA-33 | SYAMS | 633 | AISGSGG | 648 | ALVGATG | 673 |
| | | | STYYADS | | AFDI | |
| | | | VKG | | | |
| BCMA-34 | SYAMS | 633 | AISGSGG | 648 | WFGEGFD | 674 |
| | | | STYYADS | | P | |
| | | | VKG | | | |
| BCMA-35 | SYAMS | 633 | AISGSGG | 648 | VGYDSSG | 675 |
| | | | STYYADS | | YYRDYYG | |
| | | | VKG | | MDV | |
| BCMA-36 | SYAMS | 633 | AISGSGG | 648 | MGWSSGY | 676 |
| | | | STYYADS | | LGAFDI | |
| | | | VKG | | | |
| BCMA-37 | NFGMN | 636 | WINTYTG | 652 | GEIYYGY | 677 |
| | | | ESYFAD | | DGGFAY | |
| | | | DFKG | | | |
| BCMA-38 | DYSIN | 637 | WINTETR | 653 | DYSYAMD | 678 |
| | | | EPAYAYD | | Y | |
| | | | FRG | | | |
| BCMA-39 | HYSMN | 638 | RINTESG | 654 | DYLYSLD | 679 |
| | | | VPIYADD | | F | |
| | | | FKG | | | |
| BCMA-40 | HYSMN | 638 | RINTETG | 655 | DYLYSCD | 680 |
| | | | EPLYADD | | Y | |
| | | | FKG | | | |

TABLE 14F
BCMA Binders-Heavy chain CDR sequences
according to Chothia numbering scheme

| | | SEQ | | SEQ | | SEQ |
|---|---|---|---|---|---|---|
| | | ID | | ID | | ID |
| Binder | CDR-H1 | NO: | CDR-H2 | NO: | CDR-H3 | NO: |
| BCMA-1 | GFALSNH | 681 | VYSGS | 701 | HGGESDV | 656 |
| BCMA-2 | GFTFSNY | 682 | SRSGEN | 702 | SPAHYYG | 722 |
| | | | | | GMDV | |
| BCMA-3 | GFTFDDY | 683 | SWNSGS | 703 | HSFLAY | 723 |
| BCMA-4 | GFALSNH | 681 | VYSGS | 701 | HGGESDV | 656 |
| BCMA-5 | GYIFDNF | 684 | NPKNNN | 704 | GPYYYQS | 724 |
| | | | | | YMDV | |
| BCMA-6 | GFTFSSD | 685 | SGSGGT | 705 | LDSSGYY | 725 |
| | | | | | YARGPRY | |
| BCMA-7 | GYTFSNY | 686 | SAYNGN | 706 | GPYYYYM | 726 |
| | | | | | DV | |
| BCMA-8 | GFALSNH | 681 | VYSGS | 701 | HGGESDV | 656 |
| BCMA-9 | GFALSNH | 681 | VYSGS | 701 | HGGESDV | 656 |
| BCMA-10 | GFALSNH | 681 | VYSGS | 701 | HGGESDV | 656 |
| BCMA-11 | GFTFSDY | 687 | SSSGST | 707 | ESGDGMD | 727 |
| | | | | | V | |
| BCMA-12 | GFTFSDY | 687 | SSSGNT | 708 | STMVRED | 728 |
| | | | | | Y | |
| BCMA-13 | GFALSNH | 681 | VYSGS | 701 | HGGESDV | 656 |
| BCMA-14 | GFALSNH | 681 | VYSGS | 701 | HGGESDV | 656 |
| BCMA-15 | GFALSNH | 681 | VYSGS | 701 | HGGESDV | 656 |
| BCMA-16 | GGSISSSY | 688 | YYSGS | 709 | HWQEWPD | 657 |
| | | | | | AFDI | |
| BCMA-17 | GFSLRTSG | 689 | DWDED | 710 | SGAGGTS | 658 |
| | M | | | | ATAFDI | |
| BCMA-18 | GFTFSSY | 690 | SSSSSY | 711 | TIAAVYA | 659 |
| | | | | | FDI | |
| BCMA-19 | GFTFSDY | 687 | SSSGST | 707 | DLRGAFD | 660 |
| | | | | | I | |
| BCMA-20 | GYTVTSH | 691 | NPSGGV | 712 | EGSGSGW | 661 |
| | | | | | YFDF | |
| BCMA-21 | GGSISSGG | 692 | YYSGS | 709 | AGIAARL | 662 |
| | Y | | | | RGAFDI | |
| BCMA-22 | GGTFSSY | 693 | IPIFGT | 713 | RGGYQLL | 663 |
| | | | | | RWDVGLL | |
| | | | | | RS | |
| | | | | | AFDI | |
| BCMA-23 | GDSVSSNS | 694 | YYRSKWY | 714 | SSPEGLF | 664 |
| | A | | | | LYWFDP | |
| BCMA-24 | GFTFSSY | 690 | SGSGGS | 715 | VEGSGSL | 665 |
| | | | | | DY | |
| BCMA-25 | GITFSRY | 695 | SDSGVS | 716 | RAGSEAS | 666 |
| | | | | | DI | |
| BCMA-26 | GFTFSSY | 690 | SGSGGS | 715 | ATYKREL | 667 |
| | | | | | RYYYGMD | |
| | | | | | V | |
| BCMA-27 | GFTFSSY | 690 | SGSGGS | 715 | ATYKREL | 667 |
| | | | | | RYYYGMD | |
| | | | | | V | |
| BCMA-28 | GFTFDDY | 683 | SWNSGS | 703 | VGKAVPD | 668 |
| | | | | | V | |
| BCMA-29 | GFTFDDY | 683 | NWKGNS | 717 | HQGVAYY | 669 |
| | | | | | NYAMDV | |
| BCMA-30 | GFTFSSY | 690 | SGSGGS | 715 | VVRDGMD | 670 |
| | | | | | V | |
| BCMA-31 | GFTFSSY | 690 | SGSGGS | 715 | IPQTGTF | 671 |
| | | | | | DY | |
| BCMA-32 | GFTFSSY | 690 | SGSGGS | 715 | ANYKREL | 672 |
| | | | | | RYYYGMD | |
| | | | | | V | |
| BCMA-33 | GFSFSSY | 696 | SGSGGS | 715 | ALVGATG | 673 |
| | | | | | AFDI | |
| BCMA-34 | GFTFSSY | 690 | SGSGGS | 715 | WFGEGFD | 674 |
| | | | | | P | |
| BCMA-35 | GFTFSSY | 690 | SGSGGS | 715 | VGYDSSG | 675 |
| | | | | | YYRDYYG | |
| | | | | | MDV | |
| BCMA-36 | GFTFSSY | 690 | SGSGGS | 715 | MGWSSGY | 676 |
| | | | | | LGAFDI | |
| BCMA-37 | GYTFTNF | 697 | NTYTGE | 718 | GEIYYGY | 677 |
| | | | | | DGGFAY | |
| BCMA-38 | GYTFTDY | 698 | NTETRE | 719 | DYSYAMD | 678 |
| | | | | | Y | |
| BCMA-39 | GYTFRHY | 699 | NTESGV | 720 | DYLYSLD | 679 |
| | | | | | F | |
| BCMA-40 | GYTFTHY | 700 | NTETGE | 721 | DYLYSCD | 680 |
| | | | | | Y | |

TABLE 14G
BCMA Binders-Heavy chain CDR sequences
according to combination of Kabat and
Chothia numbering schemes

| | | SEQ | | SEQ | | SEQ |
|---|---|---|---|---|---|---|
| | | ID | | ID | | ID |
| Binder | CDR-H1 | NO: | CDR-H2 | NO: | CDR-H3 | NO: |
| BCMA-1 | GFAL | 729 | GIVYSGS | 639 | HGGESDV | 656 |
| | SNHG | | TYYAA | | | |
| | MS | | SVKG | | | |
| BCMA-2 | GFTF | 730 | GISRSGE | 750 | SPAHYYG | 722 |
| | SNYA | | NTYYA | | GMDV | |
| | MS | | DSVKG | | | |
| BCMA-3 | GFTF | 731 | GISWNSG | 650 | HSFLAY | 723 |
| | DDYA | | SIGYA | | | |
| | MH | | DSVKG | | | |
| BCMA-4 | GFAL | 729 | GIVYSGS | 639 | HGGESDV | 656 |
| | SNHG | | TYYAA | | | |
| | MS | | SVKG | | | |
| BCMA-5 | GYIF | 732 | WINPKNN | 751 | GPYYYQS | 724 |
| | DNFG | | NTNYA | | YMDV | |
| | IN | | QKFQG | | | |
| BCMA-6 | GFTF | 733 | VISGSGG | 752 | LDSSGYY | 725 |
| | SSDA | | TTYYA | | YARGPR | |
| | MT | | DSVKG | | Y | |
| BCMA-7 | GYTF | 734 | WISAYNG | 753 | GPYYYYM | 726 |
| | SNYG | | NTNYA | | DV | |
| | IT | | QKFQG | | | |
| BCMA-8 | GFAL | 729 | GIVYSGS | 639 | HGGESDV | 656 |
| | SNHG | | TYYAA | | | |
| | MS | | SVKG | | | |
| BCMA-9 | GFAL | 729 | GIVYSGS | 639 | HGGESDV | 656 |
| | SNHG | | TYYAA | | | |
| | MS | | SVKG | | | |
| BCMA- | GFAL | 729 | GIVYSGS | 639 | HGGESDV | 656 |
| 10 | SNHG | | TYYAA | | | |
| | MS | | SVKG | | | |
| BCMA- | GFTF | 735 | YISSSGS | 643 | ESGDGMD | 727 |
| 11 | SDYY | | TIYYAD | | V | |
| | MS | | SVKG | | | |
| BCMA- | GFTF | 735 | YISSSGN | 754 | STMVRED | 728 |
| 12 | SDYY | | TIYYAD | | Y | |
| | MS | | SVKG | | | |
| BCMA- | GFAL | 729 | GIVYSGS | 639 | HGGESDV | 656 |
| 13 | SNHG | | TYYAA | | | |
| | MS | | SVKG | | | |
| BCMA- | GFAL | 729 | GIVYSGS | 639 | HGGESDV | 656 |
| 14 | SNHG | | TYYAA | | | |
| | MS | | SVKG | | | |
| BCMA- | GFAL | 729 | GIVYSGS | 639 | HGGESDV | 656 |
| 15 | SNHG | | TYYAA | | | |
| | MS | | SVKG | | | |
| BCMA- | GGSI | 736 | SIYYSGS | 640 | HWQEWPD | 657 |
| 16 | SSSY | | AYYNP | | AFDI | |
| | Y | | | | | |
| | YWG | | SLKS | | | |
| BCMA- | GFSL | 737 | RIDWDED | 641 | SGAGGTS | 658 |
| 17 | RTSG | | KFYST | | ATAFDI | |
| | MCVS | | SLKT | | | |
| BCMA- | GFTF | 738 | SISSSSS | 642 | TIAAVYA | 659 |
| 18 | SSYS | | YIYYAD | | FDI | |
| | MN | | SVKG | | | |
| BCMA- | GFTF | 735 | YISSSGS | 643 | DLRGAFD | 660 |
| 19 | SDYY | | TIYYAD | | I | |
| | MS | | SVKG | | | |
| BCMA- | GYTV | 739 | MINPSGG | 644 | EGSGSGW | 661 |
| 20 | TSHY | | VTAYS | | YFDF | |
| | IH | | QTLQG | | | |
| BCMA- | GGSI | 740 | YIYYSGS | 645 | AGIAARL | 662 |
| 21 | SSGG | | TYYNP | | RGAFDI | |
| | YYWS | | SLKS | | | |
| BCMA- | GGTF | 741 | GHPIFGT | 646 | RGGYQLL | 663 |
| 22 | SSYA | | ANYAQ | | RWDVGL | |
| | IS | | KFQG | | LRSAFDI | |
| BCMA- | GDSV | 742 | RTYYRSK | 647 | SSPEGLF | 664 |
| 23 | SSNS | | WYSF | | LYWFDP | |
| | AAWN | | YAISLKS | | | |
| BCMA- | GFTF | 743 | AISGSGG | 648 | VEGSGSL | 665 |
| 24 | SSYA | | STYYA | | DY | |
| | MS | | DSVKG | | | |
| BCMA- | GITF | 744 | GISDSGV | 649 | RAGSEAS | 666 |
| 25 | SRYP | | STYYA | | DI | |
| | MS | | DSAKG | | | |
| BCMA- | GFTF | 743 | AISGSGG | 648 | ATYKREL | 667 |
| 26 | SSYA | | STYYA | | RYYYGM | |
| | MS | | DSVKG | | DV | |
| BCMA- | GFTF | 743 | AISGSGG | 648 | ATYKREL | 667 |
| 27 | SSYA | | STYYA | | RYYYGM | |
| | MS | | DSVKG | | DV | |
| BCMA- | GFTF | 731 | GISWNSG | 650 | VGKAVPD | 668 |
| 28 | DDYA | | SIGYA | | V | |
| | MH | | DSVKG | | | |
| BCMA- | GFTF | 731 | SINWKGN | 651 | HQGVAYY | 669 |
| 29 | DDYA | | SLAYG | | NYAMDV | |
| | MH | | DSVKG | | | |
| BCMA- | GFTF | 743 | AISGSGG | 648 | VVRDGMD | 670 |
| 30 | SSYA | | STYYA | | V | |
| | MS | | DSVKG | | | |
| BCMA- | GFTF | 743 | AISGSGG | 648 | IPQTGTF | 671 |
| 31 | SSYA | | STYYA | | DY | |
| | MS | | DSVKG | | | |
| BCMA- | GFTF | 743 | AISGSGG | 648 | ANYKREL | 672 |
| 32 | SSYA | | STYYA | | RYYYGM | |
| | MS | | DSVKG | | DV | |
| BCMA- | GFSF | 745 | AISGSGG | 648 | ALVGATG | 673 |
| 33 | SSYA | | STYYA | | AFDI | |
| | MS | | DSVKG | | | |
| BCMA- | GFTF | 743 | AISGSGG | 648 | WFGEGFD | 674 |
| 34 | SSYA | | STYYA | | P | |
| | MS | | DSVKG | | | |
| BCMA- | GFTF | 743 | AISGSGG | 648 | VGYDSSG | 675 |
| 35 | SSYA | | STYYA | | YYRDYY | |
| | MS | | DSVKG | | GMDV | |
| BCMA- | GFTF | 743 | AISGSGG | 648 | MGWSSGY | 676 |
| 36 | SSYA | | STYYA | | LGAFDI | |
| | MS | | DSVKG | | | |
| BCMA- | GYTF | 746 | WINTYTG | 652 | GEIYYGY | 677 |
| 37 | TNFG | | ESYFA | | DGGFAY | |
| | MN | | DDFKG | | | |
| BCMA- | GYTF | 747 | WINTETR | 653 | DYSYAMD | 678 |
| 38 | TDYS | | EPAYA | | Y | |
| | IN | | YDFRG | | | |
| BCMA- | GYTF | 748 | RINTESG | 654 | DYLYSLD | 679 |
| 39 | RHYS | | VPIYAD | | F | |
| | MN | | DFKG | | | |
| BCMA- | GYTF | 749 | RINTETG | 655 | DYLYSCD | 680 |
| 40 | THYS | | EPLYA | | Y | |
| | MN | | DDFKG | | | |

In some embodiments, the BCMA ABM (e.g., ABM2 or ABM5) comprises the CDR sequences of BCMA-1. In some embodiments, the BCMA ABM comprises the CDR sequences of BCMA-2. In some embodiments, the BCMA ABM comprises the CDR sequences of BCMA-3. In some embodiments, the BCMA ABM comprises the CDR sequences of BCMA-4. In some embodiments, the BCMA ABM comprises the CDR sequences of BCMA-5. In some embodiments, the BCMA ABM comprises the CDR sequences of BCMA-6. In some embodiments, the BCMA ABM comprises the CDR sequences of BCMA-7. In some embodiments, the BCMA ABM comprises the CDR sequences of BCMA-8. In some embodiments, the BCMA ABM comprises the CDR sequences of BCMA-9. In some embodiments, the BCMA ABM comprises the CDR sequences of BCMA-10. In some embodiments, the BCMA ABM comprises the CDR sequences of BCMA-11. In some embodiments, the BCMA ABM comprises the CDR sequences of BCMA-12. In some embodiments, the BCMA ABM comprises the CDR sequences of BCMA-13. In some embodiments, the BCMA ABM comprises the CDR sequences of BCMA-14. In some embodiments, the BCMA ABM comprises the CDR sequences of BCMA-15. In some embodiments, the BCMA ABM comprises the CDR sequences of BCMA-16. In some embodiments, the BCMA ABM comprises the CDR sequences of BCMA-17. In some embodiments, the BCMA ABM comprises the CDR sequences of BCMA-18. In some embodiments, the BCMA ABM comprises the CDR sequences of BCMA-19. In some embodiments, the BCMA ABM comprises the CDR sequences of BCMA-20. In some embodiments, the BCMA ABM comprises the CDR sequences of BCMA-21. In some embodiments, the BCMA ABM comprises the CDR sequences of BCMA-22. In some embodiments, the BCMA ABM comprises the CDR sequences of BCMA-23. In some embodiments, the BCMA ABM comprises the CDR sequences of BCMA-24. In some embodiments, the BCMA ABM comprises the CDR sequences of BCMA-25. In some embodiments, the BCMA ABM comprises the CDR sequences of BCMA-26. In some embodiments, the BCMA ABM comprises the CDR sequences of BCMA-27. In some embodiments, the BCMA ABM comprises the CDR sequences of BCMA-28. In some embodiments, the BCMA ABM comprises the CDR sequences of BCMA-29. In some embodiments, the BCMA ABM comprises the CDR sequences of BCMA-30. In some embodiments, the BCMA ABM comprises the CDR sequences of BCMA-31. In some embodiments, the BCMA ABM comprises the CDR sequences of BCMA-32. In some embodiments, the BCMA ABM comprises the CDR sequences of BCMA-33. In some embodiments, the BCMA ABM comprises the CDR sequences of BCMA-34. In some embodiments, the BCMA ABM comprises the CDR sequences of BCMA-35. In some embodiments, the BCMA ABM comprises the CDR sequences of BCMA-36. In some embodiments, the BCMA ABM comprises the CDR sequences of BCMA-37. In some embodiments, the BCMA ABM comprises the CDR sequences of BCMA-38. In some embodiments, the BCMA ABM comprises the CDR sequences of BCMA-39. In some embodiments, the BCMA ABM comprises the CDR sequences of BCMA-40.

In some embodiments, the CDRs are defined by Kabat numbering, as set forth in Table 14B and 14E. In other embodiments, the CDRs are defined by Chothia numbering, as set forth in Table 14C and 14F. In yet other embodiments, the CDRs are defined by a combination of Kabat and Chothia numbering, as set forth in Table 14D and 14G.

In some embodiments, the MBMs comprising a ABM that binds to BCMA can comprise the heavy and light chain variable sequences of any of BCMA-1 to BCMA-40.

In some embodiments, the BCMA ABM comprises the heavy and light chain variable sequences of BCMA-1, as set forth in Table 14A. In some embodiments, the BCMA ABM comprises the heavy and light chain variable sequences of BCMA-2, as set forth in Table 14A. In some embodiments, the BCMA ABM comprises the heavy and light chain variable sequences of BCMA-3, as set forth in Table 14A. In some embodiments, the BCMA ABM comprises the heavy and light chain variable sequences of BCMA-4, as set forth in Table 14A. In some embodiments, the BCMA ABM comprises the heavy and light chain variable sequences of BCMA-5, as set forth in Table 14A. In some embodiments, the BCMA ABM comprises the heavy and light chain variable sequences of BCMA-6, as set forth in Table 14A. In some embodiments, the BCMA ABM comprises the heavy and light chain variable sequences of BCMA-7, as set forth in Table 14A. In some embodiments, the BCMA ABM comprises the heavy and light chain variable sequences of BCMA-8, as set forth in Table 14A. In some embodiments, the BCMA ABM comprises the heavy and light chain variable sequences of BCMA-9, as set forth in Table 14A. In some embodiments, the BCMA ABM comprises the heavy and light chain variable sequences of BCMA-10, as set forth in Table 14A. In some embodiments, the BCMA ABM comprises the heavy and light chain variable sequences of BCMA-11, as set forth in Table 14A. In some embodiments, the BCMA ABM comprises the heavy and light chain variable sequences of BCMA-12, as set forth in Table 14A. In some embodiments, the BCMA ABM comprises the heavy and light chain variable sequences of BCMA-13, as set forth in Table 14A. In some embodiments, the BCMA ABM comprises the heavy and light chain variable sequences of BCMA-14, as set forth in Table 14A. In some embodiments, the BCMA ABM comprises the heavy and light chain variable sequences of BCMA-15, as set forth in Table 14A. In some embodiments, the BCMA ABM comprises the heavy and light chain variable sequences of BCMA-16, as set forth in Table 14A. In some embodiments, the BCMA ABM comprises the heavy and light chain variable sequences of BCMA-17, as set forth in Table 14A. In some embodiments, the BCMA ABM comprises the heavy and light chain variable sequences of BCMA-18, as set forth in Table 14A. In some embodiments, the BCMA ABM comprises the heavy and light chain variable sequences of BCMA-19, as set forth in Table 14A. In some embodiments, the BCMA ABM comprises the heavy and light chain variable sequences of BCMA-20, as set forth in Table 14A. In some embodiments, the BCMA ABM comprises the heavy and light chain variable sequences of BCMA-21, as set forth in Table 14A. In some embodiments, the BCMA ABM comprises the heavy and light chain variable sequences of BCMA-22, as set forth in Table 14A.

In some embodiments, the BCMA ABM comprises the heavy and light chain variable sequences of BCMA-23, as set forth in Table 14A. In some embodiments, the BCMA ABM comprises the heavy and light chain variable sequences of BCMA-24, as set forth in Table 14A. In some embodiments, the BCMA ABM comprises the heavy and light chain variable sequences of BCMA-25, as set forth in Table 14A. In some embodiments, the BCMA ABM comprises the heavy and light chain variable sequences of BCMA-26, as set forth in Table 14A. In some embodiments, the BCMA ABM comprises the heavy and light chain variable sequences of BCMA-27, as set forth in Table 14A. In some embodiments, the BCMA ABM comprises the heavy and light chain variable sequences of BCMA-28, as set forth in Table 14A. In some embodiments, the BCMA ABM comprises the heavy and light chain variable sequences of BCMA-29, as set forth in Table 14A. In some embodiments, the BCMA ABM comprises the heavy and light chain variable sequences of BCMA-30, as set forth in Table 14A. In some embodiments, the BCMA ABM comprises the heavy and light chain variable sequences of BCMA-31, as set forth in Table 14A. In some embodiments, the BCMA ABM comprises the heavy and light chain variable sequences of BCMA-32, as set forth in Table 14A. In some embodiments, the BCMA ABM comprises the heavy and light chain variable sequences of BCMA-33, as set forth in Table 14A. In some embodiments, the BCMA ABM comprises the heavy and light chain variable sequences of BCMA-34, as set forth in Table 14A. In some embodiments, the BCMA ABM comprises the heavy and light chain variable sequences of BCMA-35, as set forth in Table 14A. In some embodiments, the BCMA ABM comprises the heavy and light chain variable sequences of BCMA-36, as set forth in Table 14A. In some embodiments, the BCMA ABM comprises the heavy and light chain variable sequences of BCMA-37, as set forth in Table 14A. In some embodiments, the BCMA ABM comprises the heavy and light chain variable sequences of BCMA-38, as set forth in Table 14A. In some embodiments, the BCMA ABM comprises the heavy and light chain variable sequences of BCMA-39, as set forth in Table 14A. In some embodiments, the BCMA ABM comprises the heavy and light chain variable sequences of BCMA-40, as set forth in Table 14A.

Tables 15A-1 to 15P (collectively “Table 15”) list the sequences of additional exemplary BCMA binding sequences that can be included in a BCMA ABM (e.g., an ABM2 or an ABM5).

TABLE 15A-1
AB1/AB2 Family Light Chain CDR Consensus sequences

| | | SEQ | | SEQ | | SEQ |
|---|---|---|---|---|---|---|
| | | ID | | ID | | ID |
| Binder | CDR-L1 | NO: | CDR-L2: | NO: | CDR-L3 | NO: |
| C1 | RASQSISSYLN | 450 | AASSLQS | 482 | QQSYSXPLT | 1078 |
| (AB1/AB2 | | | | | (X = S or T) | |
| consensus- | | | | | | |
| Kabat) | | | | | | |
| C2 | RASQSISSYLN | 450 | AASSLQS | 482 | QQSYX₁X₂PX₃T | 1079 |
| (AB1/AB2 | | | | | (X₁ = S, G, D, Y, or A; X₂ = S, | |
| family | | | | | T, or A; X₃ = P or L) | |
| consensus- | | | | | | |
| Kabat) | | | | | | |
| C3 | SQSISSY | 546 | AAS | 577 | SYSXPL | 1080 |
| (AB1/AB2 | | | | | (X = S or T) | |
| consensus- | | | | | | |
| Chothia) | | | | | | |
| C4 | SQSISSY | 546 | AAS | 577 | SYX₁X₂PX₃ | 1081 |
| (AB1/AB2 | | | | | (X₁ = S, G, D, Y, or A; X₂ = S, | |
| family | | | | | T, or A; X₃ = P or L) | |
| consensus- | | | | | | |
| Chothia) | | | | | | |
| C5 | QSISSY | 1077 | AAS | 577 | QQSYSXPLT | 1078 |
| (AB1/AB2 | | | | | (X = S or T) | |
| consensus- | | | | | | |
| IMGT) | | | | | | |
| C6 | QSISSY | 1077 | AAS | 577 | QQSYX₁X₂PX₃T | 1079 |
| (AB1/AB2 | | | | | (X₁ = S, G, D, Y, or A; X₂ = S, | |
| family | | | | | T, or A; X₃ = P or L) | |
| consensus- | | | | | | |
| IMGT) | | | | | | |
| C7 | RASQSISSYLN | 450 | AASSLQS | 482 | QQSYSXPLT | 1078 |
| (AB1/AB2 | | | | | (X = S or T) | |
| consensus- | | | | | | |
| Kabat + | | | | | | |
| Chothia) | | | | | | |
| C8 | RASQSISSYLN | 450 | AASSLQS | 482 | QQSYX₁X₂PX₃T | 1079 |
| (AB1/AB2 | | | | | (X₁ = S, G, D, Y, or A; X₂ = S, | |
| family | | | | | T, or A; X₃ = P or L) | |
| consensus- | | | | | | |
| Kabat + | | | | | | |
| Chothia) | | | | | | |
| C9 | RASQSISSYLN | 450 | AASSLQS | 482 | QQSYSXPLT | 1078 |
| (AB1/AB2 | | | | | (X = S or T) | |
| consensus- | | | | | | |
| Kabat + | | | | | | |
| IMGT) | | | | | | |
| C10 | RASQSISSYLN | 450 | AASSLQS | 482 | QQSYX₁X₂PX₃T | 1079 |
| (AB1/AB2 | | | | | (X₁ = S, G, D, Y, or A; X₂ = S, | |
| family | | | | | T, or A; X₃ = P or L) | |
| consensus- | | | | | | |
| Kabat + | | | | | | |
| IMGT) | | | | | | |
| C11 | SQSISSY | 546 | AAS | 577 | QQSYSXPLT | 1078 |
| (AB1/AB2 | | | | | (X = S or T) | |
| consensus- | | | | | | |
| Chothia + | | | | | | |
| IMGT) | | | | | | |
| C12 | SQSISSY | 546 | AAS | 577 | QQSYX₁X₂PX₃T | 1079 |
| (AB1/AB2 | | | | | (X₁ = S, G, D, Y, or A; X₂ = S, | |
| family | | | | | T, or A; X₃ = P or L) | |
| consensus- | | | | | | |
| Chothia + | | | | | | |
| IMGT) | | | | | | |

TABLE 15A-2
AB1/AB2 Family Heavy Chain CDR Consensus sequences

| | | SEQ | | SEQ | | SEQ |
|---|---|---|---|---|---|---|
| | | ID | | ID | | ID |
| Binder | CDR-H1 | NO: | CDR-H2: | NO: | CDR-H3 | NO: |
| C1 | SYAMS | 633 | AISX₁SGGX₂X₃X₄YADSVKG | 1083 | REWWYDDWYLDY | 1092 |
| (AB1/AB2 | | | (X₁ = G or E; X₂ = S or R; X₃ | | | |
| consensus- | | | = T or A; X₄ = Y or A) | | | |
| Kabat) | | | | | | |
| C2 | SYAMS | 633 | AISX₁X₂GX₃X₄X₅X₆YADSVK | 1084 | REWWYDDWYLDY | 1092 |
| (AB1/AB2 | | | G | | | |
| family | | | (X₁ = G, E, or A; X₂ = S, A, | | | |
| consensus- | | | H, or E; X₃ = G, D, E , H, R, | | | |
| Kabat) | | | or A; X₄ = S, R, V, T, Y; X₅ = | | | |
| | | | T, A, E, H, or R; X₆ = Y, A, | | | |
| | | | or S) | | | |
| C3 | GFTFSSY | 690 | SX₁SGGX₂ | 1085 | REWWYDDWYLDY | 1092 |
| (AB1/AB2 | | | (X₁ = G or E; X₂ = S or R) | | | |
| consensus- | | | | | | |
| Chothia) | | | | | | |
| C4 | GFTFSSY | 690 | SX₁X₂GX₃X₄ | 1086 | REWWYDDWYLDY | 1092 |
| (AB1/AB2 | | | (X₁ = G, E, or A; X₂ = S, A, | | | |
| family | | | H, or E; X₃ = G, D, E, H, R, | | | |
| consensus- | | | or A; X₄ = S, R, V, T, Y) | | | |
| Chothia) | | | | | | |
| C5 | GFTFSSYA | 1082 | ISX₁SGGX₂X₃ | 1087 | ARREWWYDDWYL | 1093 |
| (AB1/AB2 | | | (X₁ = G or E; X₂ = S or R; X₃ | | DY | |
| consensus- | | | = T or A) | | | |
| IMGT) | | | | | | |
| C6 | GFTFSSYA | 1082 | ISX₁X₂GX₃X₄X₅ | 1088 | ARREWWYDDWYL | 1093 |
| (AB1/AB2 | | | (X₁ = G, E, or A; X₂ = S, A, | | DY | |
| family | | | H, or E; X₃ = G, D, E , H, R, | | | |
| consensus- | | | or A; X₄ = S, R, V, T, Y; X₅ = | | | |
| IMGT) | | | T, A, E, H, or R) | | | |
| C7 | GFTFSSYA | 743 | AISX₁SGGX₂X₃X₄YADSVKG | 1083 | REWWYDDWYLDY | 1092 |
| (AB1/AB2 | MS | | (X₁ = G or E; X₂ = S or R; X₃ | | | |
| consensus- | | | = T or A; X₄ = Y or A) | | | |
| Kabat + | | | | | | |
| Chothia) | | | | | | |
| C8 | GFTFSSYA | 743 | AISX₁X₂GX₃X₄X₅X₆YADSVK | 1084 | REWWYDDWYLDY | 1092 |
| (AB1/AB2 | MS | | G | | | |
| family | | | (X₁ = G, E, or A; X₂ = S, A, | | | |
| consensus- | | | H, or E; X₃ = G, D, E , H, R, | | | |
| Kabat + | | | or A; X₄ = S, R, V, T, Y; X₅ = | | | |
| Chothia) | | | T, A, E, H, or R; X₆ = Y, A, | | | |
| | | | or S) | | | |
| C9 | GFTFSSYA | 743 | AISX₁SGGX₂X₃X₄YADSVKG | 1083 | ARREWWYDDWYL | 1093 |
| (AB1/AB2 | MS | | (X₁ = G or E; X₂ = S or R; X₃ | | DY | |
| consensus- | | | = T or A; X₄ = Y or A) | | | |
| Kabat + | | | | | | |
| IMGT) | | | | | | |
| C10 | GFTFSSYA | 743 | AISX₁X₂GX₃X₄X₅X₆YADSVK | 1089 | ARREWWYDDWYL | 1093 |
| (AB1/AB2 | MS | | G | | DY | |
| family | | | (X₁ = G, E, or A; X₂ = S, A, | | | |
| consensus- | | | H, or E; X₃ = G, D, E , H, R, | | | |
| Kabat + | | | or A; X₄ = S, R, V, T, Y; X₅ = | | | |
| IMGT) | | | T, A, E, H, or R; X₆ = Y, A, | | | |
| | | | or S) | | | |
| C11 | GFTFSSYA | 1082 | ISX₁SGGX₂X₃ | 1090 | ARREWWYDDWYL | 1093 |
| (AB1/AB2 | | | (X₁ = G or E, X₂ = S or R; X₃ | | DY | |
| consensus- | | | = T or A) | | | |
| Chothia + | | | | | | |
| IMGT) | | | | | | |
| C12 | GFTFSSYA | 1082 | ISX₁X₂GX₃X₄X₅ | 1091 | ARREWWYDDWYL | 1093 |
| (AB1/AB2 | | | (X₁ = G, E, or A; X₂ = S, A, | | DY | |
| family | | | H, or E; X₃ = G, D, E , H, R, | | | |
| consensus- | | | or A; X₄ = S, R, V, T, Y; X₅ = | | | |
| Chothia + | | | T, A, E, H, or R) | | | |
| IMGT) | | | | | | |

TABLE 15B-1
AB3 Family Light Chain CDR Consensus sequences

| | | SEQ | | SEQ | | SEQ |
|---|---|---|---|---|---|---|
| | | ID | | ID | | ID |
| Binder | CDR-L1 | NO: | CDR-L2: | NO: | CDR-L3 | NO: |
| C13 | TGTSSDVGGYNYVS | 1094 | DVSNRX₁X₂ | 1097 | SSYTSSSXLYV | 1105 |
| (AB3/PI-61 | | | (X₁ = L or P; X₂ = R | | (X = A or T) | |
| consensus- | | | or S) | | | |
| Kabat) | | | | | | |
| C14 | TGTSSDVGGYNYVS | 1094 | X₁VSNRX₂X₃ | 1098 | SSYTSSSXLYV | 1105 |
| (AB3 family | | | (X₁ = D or E; X₂ = L, | | (X = A or T) | |
| consensus- | | | P, or A; X₃ = R, S, G, | | | |
| Kabat) | | | or W) | | | |
| C15 | TSSDVGGYNY | 1095 | DVS | 1099 | YTSSSXLY | 1106 |
| (AB3/PI-61 | | | | | (X = A or T) | |
| consensus- | | | | | | |
| Chothia) | | | | | | |
| C16 | TSSDVGGYNY | 1095 | XVS | 1100 | YTSSSXLY | 1106 |
| (AB3 family | | | (X = D or E) | | (X = A or T) | |
| consensus- | | | | | | |
| Chothia) | | | | | | |
| C17 | SSDVGGYNY | 1096 | DVSNRX₁X₂GVS | 1101 | SSYTSSSXLYV | 1105 |
| (AB3/PI-61 | | | (X₁ = L OR P; X₂ = R | | (X = A or T) | |
| consensus- | | | OR S) | | | |
| IMGT with | | | | | | |
| expanded | | | | | | |
| CDR-L2) | | | | | | |
| C18 | SSDVGGYNY | 1096 | X₁VSNRX₂X₃GVS | 1102 | SSYTSSSXLYV | 1105 |
| (AB3 family | | | (X₁ = D or E; X₂ = L, | | (X = A or T) | |
| consensus- | | | P, or A; X₃ = R, S, G, | | | |
| IMGT with | | | or W) | | | |
| expanded | | | | | | |
| CDR-L2) | | | | | | |
| C19 | TGTSSDVG | 1094 | DVSNRX₁X₂ | 1097 | SSYTSSSXLYV | 1105 |
| (AB3/PI-61 | GYNYVS | | (X₁ = L OR P; X₂ = R | | (X = A or T) | |
| consensus- | | | OR S) | | | |
| Kabat + | | | | | | |
| Chothia) | | | | | | |
| C20 | TGTSSDV | 1094 | X₁VSNRX₂X₃ | 1098 | SSYTSSSXLYV | 1105 |
| (AB3 family | GGYNYVS | | (X₁ = D or E; X₂ = L, | | (X = A or T) | |
| consensus- | | | P, or A; X₃ = R, S, G, | | | |
| Kabat + | | | or W) | | | |
| Chothia) | | | | | | |
| C21 | TGTSSDV | 1094 | DVSNRX₁X₂ | 1097 | SSYTSSSXLYV | 1105 |
| (AB3/PI-61 | GGYNYVS | | (X₁ = L OR P; X₂ = R | | (X = A or T) | |
| consensus- | | | OR S) | | | |
| Kabat + | | | | | | |
| IMGT) | | | | | | |
| C22 | TGTSSDVG | 1094 | X₁VSNRX₂X₃ | 1098 | SSYTSSSXLYV | 1105 |
| (AB3 family | GYNYVS | | (X₁ = D or E; X₂ = L, | | (X = A or T) | |
| consensus- | | | P, or A; X₃ = R, S, G, | | | |
| Kabat + | | | or W) | | | |
| IMGT) | | | | | | |
| C23 | TSSDVGGYNY | 1095 | DVSNRXX₂GVS | 1103 | SSYTSSSXLYV | 1105 |
| (AB3/PI-61 | | | (X₁ = L or P; X₂ = R | | (X = A or T) | |
| consensus- | | | or S) | | | |
| Chothia + | | | | | | |
| IMGT with | | | | | | |
| expanded | | | | | | |
| CDR-L2) | | | | | | |
| C24 | TSSDVGGYNY | 1095 | X₁VSNRX₂X₃GVS | 1102 | SSYTSSSXLYV | 1105 |
| (AB3 family | | | (X₁ = D or E; X₂ = L, | | (X = A or T) | |
| consensus- | | | P, or A; X₃ = R, S, G, | | | |
| Chothia + | | | or W) | | | |
| IMGT with | | | | | | |
| expanded | | | | | | |
| CDR-L2) | | | | | | |
| C25 | SSDVGGYNY | 1096 | DVS | 1099 | SSYTSSSXLYV | 1105 |
| (AB3/PI-61 | | | | | (X = A or T) | |
| consensus- | | | | | | |
| IMGT) | | | | | | |
| C26 | SSDVGGYNY | 1096 | X₁VS | 1104 | SSYTSSSXLYV | 1105 |
| (AB3 family | | | (X₁ = D or E) | | (X = A or T) | |
| consensus- | | | | | | |
| IMGT) | | | | | | |
| C27 | TSSDVGGYNY | 1095 | DVS | 1099 | SSYTSSSXLYV | 1105 |
| (AB3/PI-61 | | | | | (X = A or T) | |
| consensus- | | | | | | |
| Chothia + | | | | | | |
| IMGT) | | | | | | |
| C28 | TSSDVGGYNY | 1095 | X₁VS | 1104 | SSYTSSSXLYV | 1105 |
| (AB3 family | | | (X₁ = D or E) | | (X = A or T) | |
| consensus- | | | | | | |
| Chothia + | | | | | | |
| IMGT) | | | | | | |

TABLE 15B-2
AB3 Family Heavy Chain CDR Consensus sequences

| | | SEQ | | SEQ | | SEQ |
|---|---|---|---|---|---|---|
| | | ID | | ID | | ID |
| Binder | CDR-H1 | NO: | CDR-H2: | NO: | CDR-H3 | NO: |
| C13 | SYGMH | 1107 | VISYXGSNKYYA | 1111 | SGYALHDDYYGLDV | 1117 |
| (AB3/PI-61 | | | DSVKG | | | |
| consensus-Kabat) | | | (X = T or D) | | | |
| C14 | SYGMH | 1107 | VISYX₁X₂X₃X₄KY | 1112 | SGYX₁X₂X₃X₄X₅X₆X₇ | 1118 |
| (AB3 family | | | YADSVKG | | X₈X₉DV | |
| consensus-Kabat) | | | (X₁ = H, K, T, R, | | (X₁ = A, N, E; X₂ = L, | |
| | | | D, N, S; X₂ = G, D, | | F, V, or Y; X₃ = H, Q, | |
| | | | or E; X₃ = S, T, F, | | R, or D; X₄ = D, E, G, | |
| | | | A, L; X₄ = H, N or | | or Q; X₅ = D, Q, or F; | |
| | | | K) | | X₆ = Y or Q; X₇ = Y, | |
| | | | | | K, or D; X₈ = G or P; | |
| | | | | | X₉ = L, Q, V, or T) | |
| C15 | GFTXSSY | 1108 | SYXGSN | 1113 | SGYALHDDYYGLDV | 1117 |
| (AB3/PI-61 | (X= V or F) | | (X = T or D) | | | |
| consensus-Chothia) | | | | | | |
| C16 | GFTXSSY | 1108 | SYX₁X₂X₃X₄KG | 1114 | SGYXX₂XaX₄X₅X₆X₇ | 1118 |
| (AB3 family | (X = V or F) | | (X₁ = H, K, T, R, | | X₈X₉DV | |
| consensus- | | | D, N, S; X₂ = G, D, | | (X₁ = A, N, E; X₂ = L, | |
| Chothia) | | | or E; X₃ = S, T, F, | | F, V, or Y; X₃ = H, Q, | |
| | | | A, L; X₄ = H, N or | | R, or D; X₄ = D, E, G, | |
| | | | K) | | or Q; X₅ = D, Q, or F; | |
| | | | | | X₆ = Y or Q; X₇ = Y, | |
| | | | | | K, or D; X₈ = G or P; | |
| | | | | | X₉ = L, Q, V, or T) | |
| C17 | GFTXSSYG | 1109 | ISYXGSNK (X = T | 1115 | GGSGYALHDDYYG | 1119 |
| (AB3/PI-61 | (X = V or F) | | or D) | | LDV | |
| consensus-IMGT) | | | | | | |
| C18 | GFTXSSYG | 1109 | ISYX₁X₂X₃X₄K | 1116 | GGSGYX₁X₂X₃X₄X₅X | 1120 |
| (AB3 family | (X = V or F) | | (X₁ = H, K, T, R, | | ₆X₇X₈X₉DV | |
| consensus-IMGT) | | | D, N, S; X₂ = G, D, | | (X₁ = A, N, E; X₂ = L, | |
| | | | or E; X₃ = S, T, F, | | F, V, or Y; X₃ = H, Q, | |
| | | | A, L; X₄ = H, N or | | R, or D; X₄ = D, E, G, | |
| | | | K) | | or Q; X₅ = D, Q, or F; | |
| | | | | | X₆ = Y or Q; X₇ = Y, | |
| | | | | | K, or D; X₈ = G or P; | |
| | | | | | X₉ = L, Q, V, or T) | |
| C19 | GFTXSSYGM | 1110 | VISYXGSNKYYA | 1111 | SGYALHDDYYGLDV | 1117 |
| (AB3/PI-61 | H (X = V or F) | | DSVKG | | | |
| consensus-Kabat + | | | (X = T or D) | | | |
| Chothia) | | | | | | |
| C20 | GFTXSSYGM | 1110 | VISYX₁X₂X₃X₄KY | 1112 | SGYX₁X₂X₃X₄X₅X₆X₇ | 1118 |
| (AB3 family | H (X = V or F) | | YADSVKG | | X₈X₉DV | |
| consensus-Kabat + | | | (X₁ = H, K, T, R, | | (X₁ = A, N, E; X₂ = L, | |
| Chothia) | | | D, N, S; X₂ = G, D, | | F, V, or Y; X₃ = H, Q, | |
| | | | or E; X₃ = S, T, F, | | R, or D; X₄ = D, E, G, | |
| | | | A, L; X₄ = H, N or | | or Q; X₅ = D, Q, or F; | |
| | | | K) | | X₆ = Y or Q; X₇ = Y, | |
| | | | | | K, or D; X₈ = G or P; | |
| | | | | | X₉ = L, Q, V, or T) | |
| C21 | GFTXSSYGM | 1110 | VISYXGSNKYYA | 1111 | GGSGYALHDDYYG | 1119 |
| (AB3/PI-61 | H (X = V or F) | | DSVKG | | LDV | |
| consensus-Kabat + | | | (X = T or D) | | | |
| IMGT) | | | | | | |
| C22 | GFTXSSYGM | 1110 | VISYX₁X₂X₃X₄KY | 1112 | GGSGYX₁X₂X₃X₄X₅X₆ | 1120 |
| (AB3 family | H (X = V or F) | | YADSVKG | | X₇X₈X₉DV | |
| consensus-Kabat + | | | (X₁ = H, K, T, R, | | (X₁ = A, N, E; X₂ = L, | |
| IMGT) | | | D, N, S; X₂ = G, D, | | F, V, or Y; X₃ = H, Q, | |
| | | | or E; X₃ = S, T, F, | | R, or D; X₄ = D, E, G, | |
| | | | A, L; X₄ = H, N or | | or Q; X₅ = D, Q, or F; | |
| | | | K) | | X₆ = Y or Q; X₇ = Y, | |
| | | | | | K, or D; X₈ = G or P; | |
| | | | | | X₉ = L, Q, V, or T) | |
| C23 | GFTXSSYG | 1109 | ISYXGSNK | 1115 | GGSGYALHDDYYG | 1119 |
| (AB3/PI-61 | (X = V or F) | | (X = T or D) | | LDV | |
| consensus-Chothia | | | | | | |
| + IMGT) | | | | | | |
| C24 | GFTXSSYG | 1109 | ISYX₁X₂X₃X₄K | 1116 | GGSGYX₁X₂X₃X₄X₅X₆ | 1120 |
| (AB3 family | (X = V or F) | | (X₁ = H, K, T, R, | | X₇X₈X₉DV | |
| consensus-Chothia | | | D, N, S; X₂ = G, D, | | (X₁ = A, N, E; X₂ = L, | |
| + IMGT) | | | or E; X₃ = S, T, F, | | F, V, or Y; X₃ = H, Q, | |
| | | | A, L; X₄ = H, N or | | R, or D; X₄ = D, E, G, | |
| | | | K) | | or Q; X₅ = D, Q, or F; | |
| | | | | | X₆ = Y or Q; X₇ = Y, | |
| | | | | | K, or D; X₈ = G or P; | |
| | | | | | X₉ = L, Q, V, or T) | |
| C25 | GFTXSSYG | 1109 | ISYXGSNK (X = T | 1115 | GGSGYALHDDYYG | 1119 |
| (AB3/PI-61 | (X = V or F) | | or D) | | LDV | |
| consensus-IMGT) | | | | | | |
| C26 | GFTXSSYG | 1109 | ISYX₁X₂X₃X₄K | 1116 | GGSGYX₁X₂X₃X₄X₅X₆ | 1120 |
| (AB3 family | (X = V or F) | | (X₁ = H, K, T, R, | | X₇X₈X₉DV | |
| consensus-IMGT) | | | D, N, S; X₂ = G, D, | | (X₁ = A, N, E; X₂ = L, | |
| | | | or E; X₃ = S, T, F, | | F, V, or Y; X₃ = H, Q, | |
| | | | A, L; X₄ = H, N or | | R, or D; X₄ = D, E, G, | |
| | | | K) | | or Q; X₅ = D, Q, or F; | |
| | | | | | X₆ = Y or Q; X₇ = Y, | |
| | | | | | K, or D; X₈ = G or P; | |
| | | | | | X₉ = L, Q, V, or T) | |
| C27 | GFTXSSYG | 1109 | ISYXGSNK | 1115 | GGSGYALHDDYYG | 1119 |
| (AB3/PI-61 | (X = V or F) | | (X = T or D) | | LDV | |
| consensus-Chothia | | | | | | |
| + IMGT) | | | | | | |
| C28 | GFTXSSYG | 1109 | ISYX₁X₂X₃X₄K | 1116 | GGSGYX₁X₂X₃X₄X₅X₆ | 1120 |
| (AB3 family | (X = V or F) | | (X₁ = H, K, T, R, | | X₇X₈X₉DV | |
| consensus-Chothia | | | D, N, S; X₂ = G, D, | | (X₁ = A, N, E; X₂ = L, | |
| + IMGT) | | | or E; X₃ = S, T, F, | | F, V, or Y; X₃ = H, Q, | |
| | | | A, L; X₄ = H, N or | | R, or D; X₄ = D, E, G, | |
| | | | K) | | or Q; X₅ = D, Q, or F; | |
| | | | | | X₆ = Y or Q; X₇ = Y, | |
| | | | | | K, or D; X₈ = G or P; | |
| | | | | | X₉ = L, Q, V, or T) | |

TABLE 15C-1
AB1/AB2 family BCMA Binders-Light Chain CDR sequences according to
Kabat numbering scheme

| | | SEQ ID | | SEQ ID | | SEQ ID |
|---|---|---|---|---|---|---|
| Binder | CDR-L1 | NO: | CDR-L2: | NO: | CDR-L3 | NO: |
| AB1 | RASQSISSYLN | 450 | AASSLQS | 482 | QQSYSSPLT | 1121 |
| AB2 | RASQSISSYLN | 450 | AASSLQS | 482 | QQSYSTPLT | 54 |
| R1F2 | RASQSISSYLN | 450 | AASSLQS | 482 | QQSYSTPLT | 54 |
| PALF03 | RASQSISSYLN | 450 | AASSLQS | 482 | QQSYGSPPT | 1122 |
| PALF04 | RASQSISSYLN | 450 | AASSLQS | 482 | QQSYDSPLT | 1123 |
| PALF05 | RASQSISSYLN | 450 | AASSLQS | 482 | QQSYYSPLT | 1124 |
| PALF06 | RASQSISSYLN | 450 | AASSLQS | 482 | QQSYYAPLT | 1125 |
| PALF07 | RASQSISSYLN | 450 | AASSLQS | 482 | QQSYASPLT | 1126 |
| PALF08 | RASQSISSYLN | 450 | AASSLQS | 482 | QQSYGSPLT | 1127 |
| PALF09 | RASQSISSYLN | 450 | AASSLQS | 482 | QQSYDAPLT | 1128 |
| PALF12 | RASQSISSYLN | 450 | AASSLQS | 482 | QQSYSTPLT | 54 |
| PALF13 | RASQSISSYLN | 450 | AASSLQS | 482 | QQSYSTPLT | 54 |
| PALF14 | RASQSISSYLN | 450 | AASSLQS | 482 | QQSYSTPLT | 54 |
| PALF15 | RASQSISSYLN | 450 | AASSLQS | 482 | QQSYSTPLT | 54 |
| PALF16 | RASQSISSYLN | 450 | AASSLQS | 482 | QQSYSTPLT | 54 |
| PALF17 | RASQSISSYLN | 450 | AASSLQS | 482 | QQSYSTPLT | 54 |
| PALF18 | RASQSISSYLN | 450 | AASSLQS | 482 | QQSYSTPLT | 54 |
| PALF19 | RASQSISSYLN | 450 | AASSLQS | 482 | QQSYSTPLT | 54 |
| PALF20 | RASQSISSYLN | 450 | AASSLQS | 482 | QQSYSTPLT | 54 |

TABLE 15C-2
AB1/AB2 family BCMA Binders-Heavy Chain CDR sequences according to Kabat
numbering scheme

| | | SEQ ID | | SEQ ID | | SEQ ID |
|---|---|---|---|---|---|---|
| Binder | CDR-H1 | NO: | CDR-H2: | NO: | CDR-H3 | NO: |
| AB1 | SYAMS | 633 | AISGSGGSTYYADSVKG | 648 | REWWYDDWYLDY | 1092 |
| AB2 | SYAMS | 633 | AISESGGRAAYADSVKG | 1129 | REWWYDDWYLDY | 1092 |
| R1F2 | SYAMS | 633 | AISGSGGSTYYADSVKG | 648 | REWWYDDWYLDY | 1092 |
| PALF03 | SYAMS | 633 | AISGSGGSTYYADSVKG | 648 | REWWYDDWYLDY | 1092 |
| PALF04 | SYAMS | 633 | AISGSGGSTYYADSVKG | 648 | REWWYDDWYLDY | 1092 |
| PALF05 | SYAMS | 633 | AISGSGGSTYYADSVKG | 648 | REWWYDDWYLDY | 1092 |
| PALF06 | SYAMS | 633 | AISGSGGSTYYADSVKG | 648 | REWWYDDWYLDY | 1092 |
| PALF07 | SYAMS | 633 | AISGSGGSTYYADSVKG | 648 | REWWYDDWYLDY | 1092 |
| PALF08 | SYAMS | 633 | AISGSGGSTYYADSVKG | 648 | REWWYDDWYLDY | 1092 |
| PALF09 | SYAMS | 633 | AISGSGGSTYYADSVKG | 648 | REWWYDDWYLDY | 1092 |
| PALF12 | SYAMS | 633 | AISGSGGRAAYADSVK | 1130 | REWWYDDWYLDY | 1092 |
| | | | G | | | |
| PALF13 | SYAMS | 633 | AISESGDVEAYADSVKG | 1131 | REWWYDDWYLDY | 1092 |
| PALF14 | SYAMS | 633 | AISEAGETTSYADSVKG | 1132 | REWWYDDWYLDY | 1092 |
| PALF15 | SYAMS | 633 | AISEHGHYTSYADSVKG | 1133 | REWWYDDWYLDY | 1092 |
| PALF16 | SYAMS | 633 | AISGSGHTAAYADSVKG | 1134 | REWWYDDWYLDY | 1092 |
| PALF17 | SYAMS | 633 | AISGSGRTHAYADSVKG | 1135 | REWWYDDWYLDY | 1092 |
| PALF18 | SYAMS | 633 | AISAEGGVRAYADSVKG | 1136 | REWWYDDWYLDY | 1092 |
| PALF19 | SYAMS | 633 | AISGSGGTTAYADSVKG | 1137 | REWWYDDWYLDY | 1092 |
| PALF20 | SYAMS | 633 | AISGSGATTAYADSVKG | 1138 | REWWYDDWYLDY | 1092 |

TABLE 15D-1
AB1/AB2 family BCMA Binders-Light Chain CDR sequences according
to Chothia numbering scheme

| | | SEQ ID | | SEQ ID | | SEQ ID |
|---|---|---|---|---|---|---|
| Binder | CDR-L1 | NO: | CDR-L2: | NO: | CDR-L3 | NO: |
| AB1 | SQSISSY | 546 | AAS | 577 | SYSSPL | 1139 |
| AB2 | SQSISSY | 546 | AAS | 577 | SYSTPL | 1140 |
| R1F2 | SQSISSY | 546 | AAS | 577 | SYSTPL | 1140 |
| PALF03 | SQSISSY | 546 | AAS | 577 | SYGSPP | 1141 |
| PALF04 | SQSISSY | 546 | AAS | 577 | SYDSPL | 1142 |
| PALF05 | SQSISSY | 546 | AAS | 577 | SYYSPL | 1143 |
| PALF06 | SQSISSY | 546 | AAS | 577 | SYYAPL | 78 |
| PALF07 | SQSISSY | 546 | AAS | 577 | SYASPL | 1144 |
| PALF08 | SQSISSY | 546 | AAS | 577 | SYGSPL | 1145 |
| PALF09 | SQSISSY | 546 | AAS | 577 | SYDAPL | 1146 |
| PALF12 | SQSISSY | 546 | AAS | 577 | SYSTPL | 1140 |
| PALF13 | SQSISSY | 546 | AAS | 577 | SYSTPL | 1140 |
| PALF14 | SQSISSY | 546 | AAS | 577 | SYSTPL | 1140 |
| PALF15 | SQSISSY | 546 | AAS | 577 | SYSTPL | 1140 |
| PALF16 | SQSISSY | 546 | AAS | 577 | SYSTPL | 1140 |
| PALF17 | SQSISSY | 546 | AAS | 577 | SYSTPL | 1140 |
| PALF18 | SQSISSY | 546 | AAS | 577 | SYSTPL | 1140 |
| PALF19 | SQSISSY | 546 | AAS | 577 | SYSTPL | 1140 |
| PALF20 | SQSISSY | 546 | AAS | 577 | SYSTPL | 1140 |

TABLE 15D-2
AB1/AB2 family BCMA Binders-Heavy Chain CDR sequences according to
Chothia numbering scheme

| | | SEQ ID | | SEQ ID | | SEQ ID |
|---|---|---|---|---|---|---|
| Binder | CDR-H1 | NO: | CDR-H2: | NO: | CDR-H3 | NO: |
| AB1 | GFTFSSY | 690 | SGSGGS | 715 | REWWYDDWYLDY | 1092 |
| AB2 | GFTFSSY | 690 | SESGGR | 1147 | REWWYDDWYLDY | 1092 |
| R1F2 | GFTFSSY | 690 | SGSGGS | 715 | REWWYDDWYLDY | 1092 |
| PALF03 | GFTFSSY | 690 | SGSGGS | 715 | REWWYDDWYLDY | 1092 |
| PALF04 | GFTFSSY | 690 | SGSGGS | 715 | REWWYDDWYLDY | 1092 |
| PALF05 | GFTFSSY | 690 | SGSGGS | 715 | REWWYDDWYLDY | 1092 |
| PALF06 | GFTFSSY | 690 | SGSGGS | 715 | REWWYDDWYLDY | 1092 |
| PALF07 | GFTFSSY | 690 | SGSGGS | 715 | REWWYDDWYLDY | 1092 |
| PALF08 | GFTFSSY | 690 | SGSGGS | 715 | REWWYDDWYLDY | 1092 |
| PALF09 | GFTFSSY | 690 | SGSGGS | 715 | REWWYDDWYLDY | 1092 |
| PALF12 | GFTFSSY | 690 | SGSGGR | 1148 | REWWYDDWYLDY | 1092 |
| PALF13 | GFTFSSY | 690 | SESGDV | 1149 | REWWYDDWYLDY | 1092 |
| PALF14 | GFTFSSY | 690 | SESGDV | 1149 | REWWYDDWYLDY | 1092 |
| PALF15 | GFTFSSY | 690 | SEHGHY | 1150 | REWWYDDWYLDY | 1092 |
| PALF16 | GFTFSSY | 690 | SGSGHT | 1151 | REWWYDDWYLDY | 1092 |
| PALF17 | GFTFSSY | 690 | SGSGRT | 1152 | REWWYDDWYLDY | 1092 |
| PALF18 | GFTFSSY | 690 | SAEGGV | 1153 | REWWYDDWYLDY | 1092 |
| PALF19 | GFTFSSY | 690 | SGSGGT | 705 | REWWYDDWYLDY | 1092 |
| PALF20 | GFTFSSY | 690 | SGSGAT | 1154 | REWWYDDWYLDY | 1092 |

TABLE 15E-1
AB1/AB2 family BCMA Binders-Light Chain CDR sequences according to
IMGT numbering scheme

| | | SEQ ID | | SEQ ID | | SEQ ID |
|---|---|---|---|---|---|---|
| Binder | CDR-L1 | NO: | CDR-L2: | NO: | CDR-L3 | NO: |
| AB1 | QSISSY | 1077 | AAS | 577 | QQSYSSPLT | 1121 |
| AB2 | QSISSY | 1077 | AAS | 577 | QQSYSTPLT | 54 |
| R1F2 | QSISSY | 1077 | AAS | 577 | QQSYSTPLT | 54 |
| PALF03 | QSISSY | 1077 | AAS | 577 | QQSYGSPPT | 1122 |
| PALF04 | QSISSY | 1077 | AAS | 577 | QQSYDSPLT | 1123 |
| PALF05 | QSISSY | 1077 | AAS | 577 | QQSYYSPLT | 1124 |
| PALF06 | QSISSY | 1077 | AAS | 577 | QQSYYAPLT | 1125 |
| PALF07 | QSISSY | 1077 | AAS | 577 | QQSYASPLT | 1126 |
| PALF08 | QSISSY | 1077 | AAS | 577 | QQSYGSPLT | 1127 |
| PALF09 | QSISSY | 1077 | AAS | 577 | QQSYDAPLT | 1128 |
| PALF12 | QSISSY | 1077 | AAS | 577 | QQSYSTPLT | 54 |
| PALF13 | QSISSY | 1077 | AAS | 577 | QQSYSTPLT | 54 |
| PALF14 | QSISSY | 1077 | AAS | 577 | QQSYSTPLT | 54 |
| PALF15 | QSISSY | 1077 | AAS | 577 | QQSYSTPLT | 54 |
| PALF16 | QSISSY | 1077 | AAS | 577 | QQSYSTPLT | 54 |
| PALF17 | QSISSY | 1077 | AAS | 577 | QQSYSTPLT | 54 |
| PALF18 | QSISSY | 1077 | AAS | 577 | QQSYSTPLT | 54 |
| PALF19 | QSISSY | 1077 | AAS | 577 | QQSYSTPLT | 54 |
| PALF20 | QSISSY | 1077 | AAS | 577 | QQSYSTPLT | 54 |

TABLE 15E-2
AB1/AB2 family BCMA Binders-Heavy Chain CDR sequences according to IMGT
numbering scheme

| | | SEQ ID | | SEQ ID | | SEQ ID |
|---|---|---|---|---|---|---|
| Binder | CDR-H1 | NO: | CDR-H2: | NO: | CDR-H3 | NO: |
| AB1 | GFTFSSYA | 1082 | ISGSGGST | 1155 | ARREWWYDDWYL | 1093 |
| | | | | | DY | |
| AB2 | GFTFSSYA | 1082 | ISESGGRA | 1156 | ARREWWYDDWYL | 1093 |
| | | | | | DY | |
| R1F2 | GFTFSSYA | 1082 | ISGSGGST | 1155 | ARREWWYDDWYL | 1093 |
| | | | | | DY | |
| PALF03 | GFTFSSYA | 1082 | ISGSGGST | 1155 | ARREWWYDDWYL | 1093 |
| | | | | | DY | |
| PALF04 | GFTFSSYA | 1082 | ISGSGGST | 1155 | ARREWWYDDWYL | 1093 |
| | | | | | DY | |
| PALF05 | GFTFSSYA | 1082 | ISGSGGST | 1155 | ARREWWYDDWYL | 1093 |
| | | | | | DY | |
| PALF06 | GFTFSSYA | 1082 | ISGSGGST | 1155 | ARREWWYDDWYL | 1093 |
| | | | | | DY | |
| PALF07 | GFTFSSYA | 1082 | ISGSGGST | 1155 | ARREWWYDDWYL | 1093 |
| | | | | | DY | |
| PALF08 | GFTFSSYA | 1082 | ISGSGGST | 1155 | ARREWWYDDWYL | 1093 |
| | | | | | DY | |
| PALF09 | GFTFSSYA | 1082 | ISGSGGST | 1155 | ARREWWYDDWYL | 1093 |
| | | | | | DY | |
| PALF12 | GFTFSSYA | 1082 | ISGSGGRA | 1157 | ARREWWYDDWYL | 1093 |
| | | | | | DY | |
| PALF13 | GFTFSSYA | 1082 | ISESGDVE | 1158 | ARREWWYDDWYL | 1093 |
| | | | | | DY | |
| PALF14 | GFTFSSYA | 1082 | ISESGDVE | 1158 | ARREWWYDDWYL | 1093 |
| | | | | | DY | |
| PALF15 | GFTFSSYA | 1082 | ISEHGHYT | 1159 | ARREWWYDDWYL | 1093 |
| | | | | | DY | |
| PALF16 | GFTFSSYA | 1082 | ISGSGHTA | 1160 | ARREWWYDDWYL | 1093 |
| | | | | | DY | |
| PALF17 | GFTFSSYA | 1082 | ISGSGRTH | 1161 | ARREWWYDDWYL | 1093 |
| | | | | | DY | |
| PALF18 | GFTFSSYA | 1082 | ISAEGGVR | 1162 | ARREWWYDDWYL | 1093 |
| | | | | | DY | |
| PALF19 | GFTFSSYA | 1082 | ISGSGGTT | 1163 | ARREWWYDDWYL | 1093 |
| | | | | | DY | |
| PALF20 | GFTFSSYA | 1082 | ISGSGATT | 1164 | ARREWWYDDWYL | 1093 |
| | | | | | DY | |

TABLE 15F-1
AB1/AB2 family BCMA Binders-Light Chain CDR sequences according to
combination of Kabat and Chothia numbering schemes

| | | SEQ ID | | SEQ ID | | SEQ ID |
|---|---|---|---|---|---|---|
| Binder | CDR-L1 | NO: | CDR-L2: | NO: | CDR-L3 | NO: |
| AB1 | RASQSISSYLN | 450 | AASSLQS | 482 | QQSYSSPLT | 1121 |
| AB2 | RASQSISSYLN | 450 | AASSLQS | 482 | QQSYSTPLT | 54 |
| R1F2 | RASQSISSYLN | 450 | AASSLQS | 482 | QQSYSTPLT | 54 |
| PALF03 | RASQSISSYLN | 450 | AASSLQS | 482 | QQSYGSPPT | 1122 |
| PALF04 | RASQSISSYLN | 450 | AASSLQS | 482 | QQSYDSPLT | 1123 |
| PALF05 | RASQSISSYLN | 450 | AASSLQS | 482 | QQSYYSPLT | 1124 |
| PALF06 | RASQSISSYLN | 450 | AASSLQS | 482 | QQSYYAPLT | 1125 |
| PALF07 | RASQSISSYLN | 450 | AASSLQS | 482 | QQSYASPLT | 1126 |
| PALF08 | RASQSISSYLN | 450 | AASSLQS | 482 | QQSYGSPLT | 1127 |
| PALF09 | RASQSISSYLN | 450 | AASSLQS | 482 | QQSYDAPLT | 1128 |
| PALF12 | RASQSISSYLN | 450 | AASSLQS | 482 | QQSYSTPLT | 54 |
| PALF13 | RASQSISSYLN | 450 | AASSLQS | 482 | QQSYSTPLT | 54 |
| PALF14 | RASQSISSYLN | 450 | AASSLQS | 482 | QQSYSTPLT | 54 |
| PALF15 | RASQSISSYLN | 450 | AASSLQS | 482 | QQSYSTPLT | 54 |
| PALF16 | RASQSISSYLN | 450 | AASSLQS | 482 | QQSYSTPLT | 54 |
| PALF17 | RASQSISSYLN | 450 | AASSLQS | 482 | QQSYSTPLT | 54 |
| PALF18 | RASQSISSYLN | 450 | AASSLQS | 482 | QQSYSTPLT | 54 |
| PALF19 | RASQSISSYLN | 450 | AASSLQS | 482 | QQSYSTPLT | 54 |
| PALF20 | RASQSISSYLN | 450 | AASSLQS | 482 | QQSYSTPLT | 54 |

TABLE 15F-2
AB1/AB2 family BCMA Binders-Heavy Chain CDR sequences according to combination of Kabat
and Chothia numbering schemes

| | | SEQ ID | | SEQ ID | | SEQ ID |
|---|---|---|---|---|---|---|
| Binder | CDR-H1 | NO: | CDR-H2: | NO: | CDR-H3 | NO: |
| AB1 | GFTFSSYAMS | 743 | AISGSGGSTYYA | 648 | REWWYDDWYLDY | 1092 |
| | | | DSVKG | | | |
| AB2 | GFTFSSYAMS | 743 | AISESGGRAAYA | 1129 | REWWYDDWYLDY | 1092 |
| | | | DSVKG | | | |
| R1F2 | GFTFSSYAMS | 743 | AISGSGGSTYYA | 648 | REWWYDDWYLDY | 1092 |
| | | | DSVKG | | | |
| PALF03 | GFTFSSYAMS | 743 | AISGSGGSTYYA | 648 | REWWYDDWYLDY | 1092 |
| | | | DSVKG | | | |
| PALF04 | GFTFSSYAMS | 743 | AISGSGGSTYYA | 648 | REWWYDDWYLDY | 1092 |
| | | | DSVKG | | | |
| PALF05 | GFTFSSYAMS | 743 | AISGSGGSTYYA | 648 | REWWYDDWYLDY | 1092 |
| | | | DSVKG | | | |
| PALF06 | GFTFSSYAMS | 743 | AISGSGGSTYYA | 648 | REWWYDDWYLDY | 1092 |
| | | | DSVKG | | | |
| PALF07 | GFTFSSYAMS | 743 | AISGSGGSTYYA | 648 | REWWYDDWYLDY | 1092 |
| | | | DSVKG | | | |
| PALF08 | GFTFSSYAMS | 743 | AISGSGGSTYYA | 648 | REWWYDDWYLDY | 1092 |
| | | | DSVKG | | | |
| PALF09 | GFTFSSYAMS | 743 | AISGSGGSTYYA | 648 | REWWYDDWYLDY | 1092 |
| | | | DSVKG | | | |
| PALF12 | GFTFSSYAMS | 743 | AISGSGGRAAYA | 1130 | REWWYDDWYLDY | 1092 |
| | | | DSVKG | | | |
| PALF13 | GFTFSSYAMS | 743 | AISESGDVEAYA | 1131 | REWWYDDWYLDY | 1092 |
| | | | DSVKG | | | |
| PALF14 | GFTFSSYAMS | 743 | AISEAGETTSYA | 1132 | REWWYDDWYLDY | 1092 |
| | | | DSVKG | | | |
| PALF15 | GFTFSSYAMS | 743 | AISEHGHYTSYA | 1133 | REWWYDDWYLDY | 1092 |
| | | | DSVKG | | | |
| PALF16 | GFTFSSYAMS | 743 | AISGSGHTAAYA | 1134 | REWWYDDWYLDY | 1092 |
| | | | DSVKG | | | |
| PALF17 | GFTFSSYAMS | 743 | AISGSGRTHAYA | 1135 | REWWYDDWYLDY | 1092 |
| | | | DSVKG | | | |
| PALF18 | GFTFSSYAMS | 743 | AISAEGGVRAYA | 1136 | REWWYDDWYLDY | 1092 |
| | | | DSVKG | | | |
| PALF19 | GFTFSSYAMS | 743 | AISGSGGTTAYA | 1137 | REWWYDDWYLDY | 1092 |
| | | | DSVKG | | | |
| PALF20 | GFTFSSYAMS | 743 | AISGSGATTAYA | 1138 | REWWYDDWYLDY | 1092 |
| | | | DSVKG | | | |

TABLE 15G-1
AB1/AB2 family BCMA Binders-Light Chain CDR sequences according to
combination of Kabat and IMGT numbering schemes

| | | SEQ ID | | SEQ ID | | SEQ ID |
|---|---|---|---|---|---|---|
| Binder | CDR-L1 | NO: | CDR-L2: | NO: | CDR-L3 | NO: |
| AB1 | RASQSISSYLN | 450 | AASSLQS | 482 | QQSYSSPLT | 1121 |
| AB2 | RASQSISSYLN | 450 | AASSLQS | 482 | QQSYSTPLT | 54 |
| R1F2 | RASQSISSYLN | 450 | AASSLQS | 482 | QQSYSTPLT | 54 |
| PALF03 | RASQSISSYLN | 450 | AASSLQS | 482 | QQSYGSPPT | 1122 |
| PALF04 | RASQSISSYLN | 450 | AASSLQS | 482 | QQSYDSPLT | 1123 |
| PALF05 | RASQSISSYLN | 450 | AASSLQS | 482 | QQSYYSPLT | 1124 |
| PALF06 | RASQSISSYLN | 450 | AASSLQS | 482 | QQSYYAPLT | 1125 |
| PALF07 | RASQSISSYLN | 450 | AASSLQS | 482 | QQSYASPLT | 1126 |
| PALF08 | RASQSISSYLN | 450 | AASSLQS | 482 | QQSYGSPLT | 1127 |
| PALF09 | RASQSISSYLN | 450 | AASSLQS | 482 | QQSYDAPLT | 1128 |
| PALF12 | RASQSISSYLN | 450 | AASSLQS | 482 | QQSYSTPLT | 54 |
| PALF13 | RASQSISSYLN | 450 | AASSLQS | 482 | QQSYSTPLT | 54 |
| PALF14 | RASQSISSYLN | 450 | AASSLQS | 482 | QQSYSTPLT | 54 |
| PALF15 | RASQSISSYLN | 450 | AASSLQS | 482 | QQSYSTPLT | 54 |
| PALF16 | RASQSISSYLN | 450 | AASSLQS | 482 | QQSYSTPLT | 54 |
| PALF17 | RASQSISSYLN | 450 | AASSLQS | 482 | QQSYSTPLT | 54 |
| PALF18 | RASQSISSYLN | 450 | AASSLQS | 482 | QQSYSTPLT | 54 |
| PALF19 | RASQSISSYLN | 450 | AASSLQS | 482 | QQSYSTPLT | 54 |
| PALF20 | RASQSISSYLN | 450 | AASSLQS | 482 | QQSYSTPLT | 54 |

TABLE 15G-2
AB1/AB2 family BCMA Binders-Heavy Chain CDR sequences according to combination of Kabat
and IMGT numbering schemes

| | | SEQ ID | | SEQ ID | | SEQ ID |
|---|---|---|---|---|---|---|
| Binder | CDR-H1 | NO: | CDR-H2: | NO: | CDR-H3 | NO: |
| AB1 | GFTFSSYAMS | 743 | AISGSGGSTYYA | 648 | ARREWWYDDWYL | 1093 |
| | | | DSVKG | | DY | |
| AB2 | GFTFSSYAMS | 743 | AISESGGRAAYA | 1129 | ARREWWYDDWYL | 1093 |
| | | | DSVKG | | DY | |
| R1F2 | GFTFSSYAMS | 743 | AISGSGGSTYYA | 648 | ARREWWYDDWYL | 1093 |
| | | | DSVKG | | DY | |
| PALF03 | GFTFSSYAMS | 743 | AISGSGGSTYYA | 648 | ARREWWYDDWYL | 1093 |
| | | | DSVKG | | DY | |
| PALF04 | GFTFSSYAMS | 743 | AISGSGGSTYYA | 648 | ARREWWYDDWYL | 1093 |
| | | | DSVKG | | DY | |
| PALF05 | GFTFSSYAMS | 743 | AISGSGGSTYYA | 648 | ARREWWYDDWYL | 1093 |
| | | | DSVKG | | DY | |
| PALF06 | GFTFSSYAMS | 743 | AISGSGGSTYYA | 648 | ARREWWYDDWYL | 1093 |
| | | | DSVKG | | DY | |
| PALF07 | GFTFSSYAMS | 743 | AISGSGGSTYYA | 648 | ARREWWYDDWYL | 1093 |
| | | | DSVKG | | DY | |
| PALF08 | GFTFSSYAMS | 743 | AISGSGGSTYYA | 648 | ARREWWYDDWYL | 1093 |
| | | | DSVKG | | DY | |
| PALF09 | GFTFSSYAMS | 743 | AISGSGGSTYYA | 648 | ARREWWYDDWYL | 1093 |
| | | | DSVKG | | DY | |
| PALF12 | GFTFSSYAMS | 743 | AISGSGGRAAYA | 1130 | ARREWWYDDWYL | 1093 |
| | | | DSVKG | | DY | |
| PALF13 | GFTFSSYAMS | 743 | AISESGDVEAYA | 1131 | ARREWWYDDWYL | 1093 |
| | | | DSVKG | | DY | |
| PALF14 | GFTFSSYAMS | 743 | AISEAGETTSYAD | 1132 | ARREWWYDDWYL | 1093 |
| | | | SVKG | | DY | |
| PALF15 | GFTFSSYAMS | 743 | AISEHGHYTSYA | 1133 | ARREWWYDDWYL | 1093 |
| | | | DSVKG | | DY | |
| PALF16 | GFTFSSYAMS | 743 | AISGSGHTAAYA | 1134 | ARREWWYDDWYL | 1093 |
| | | | DSVKG | | DY | |
| PALF17 | GFTFSSYAMS | 743 | AISGSGRTHAYA | 1135 | ARREWWYDDWYL | 1093 |
| | | | DSVKG | | DY | |
| PALF18 | GFTFSSYAMS | 743 | AISAEGGVRAYA | 1136 | ARREWWYDDWYL | 1093 |
| | | | DSVKG | | DY | |
| PALF19 | GFTFSSYAMS | 743 | AISGSGGTTAYA | 1137 | ARREWWYDDWYL | 1093 |
| | | | DSVKG | | DY | |
| PALF20 | GFTFSSYAMS | 743 | AISGSGATTAYA | 1138 | ARREWWYDDWYL | 1093 |
| | | | DSVKG | | DY | |

TABLE 15H-1
AB1/AB2 family BCMA Binders-Light Chain CDR sequences according to
combination of Chothia and IMGT numbering schemes

| | | SEQ ID | | SEQ ID | | SEQ ID |
|---|---|---|---|---|---|---|
| Binder | CDR-L1 | NO: | CDR-L2: | NO: | CDR-L3 | NO: |
| AB1 | SQSISSY | 546 | AAS | 577 | QQSYSSPLT | 1121 |
| AB2 | SQSISSY | 546 | AAS | 577 | QQSYSTPLT | 54 |
| R1F2 | SQSISSY | 546 | AAS | 577 | QQSYSTPLT | 54 |
| PALF03 | SQSISSY | 546 | AAS | 577 | QQSYGSPPT | 1122 |
| PALF04 | SQSISSY | 546 | AAS | 577 | QQSYDSPLT | 1123 |
| PALF05 | SQSISSY | 546 | AAS | 577 | QQSYYSPLT | 1124 |
| PALF06 | SQSISSY | 546 | AAS | 577 | QQSYYAPLT | 1125 |
| PALF07 | SQSISSY | 546 | AAS | 577 | QQSYASPLT | 1126 |
| PALF08 | SQSISSY | 546 | AAS | 577 | QQSYGSPLT | 1127 |
| PALF09 | SQSISSY | 546 | AAS | 577 | QQSYDAPLT | 1128 |
| PALF12 | SQSISSY | 546 | AAS | 577 | QQSYSTPLT | 54 |
| PALF13 | SQSISSY | 546 | AAS | 577 | QQSYSTPLT | 54 |
| PALF14 | SQSISSY | 546 | AAS | 577 | QQSYSTPLT | 54 |
| PALF15 | SQSISSY | 546 | AAS | 577 | QQSYSTPLT | 54 |
| PALF16 | SQSISSY | 546 | AAS | 577 | QQSYSTPLT | 54 |
| PALF17 | SQSISSY | 546 | AAS | 577 | QQSYSTPLT | 54 |
| PALF18 | SQSISSY | 546 | AAS | 577 | QQSYSTPLT | 54 |
| PALF19 | SQSISSY | 546 | AAS | 577 | QQSYSTPLT | 54 |
| PALF20 | SQSISSY | 546 | AAS | 577 | QQSYSTPLT | 54 |

TABLE 15H-2
AB1/AB2 family BCMA Binders-Heavy Chain CDR sequences according to combination of
Chothia and IMGT numbering schemes

| | | SEQ ID | | SEQ ID | | SEQ ID |
|---|---|---|---|---|---|---|
| Binder | CDR-H1 | NO: | CDR-H2: | NO: | CDR-H3 | NO: |
| AB1 | GFTFSSYA | 1082 | ISGSGGST | 1155 | ARREWWYDDWYL | 1093 |
| | | | | | DY | |
| AB2 | GFTFSSYA | 1082 | ISESGGRA | 1156 | ARREWWYDDWYL | 1093 |
| | | | | | DY | |
| R1F2 | GFTFSSYA | 1082 | ISGSGGST | 1155 | ARREWWYDDWYL | 1093 |
| | | | | | DY | |
| PALF03 | GFTFSSYA | 1082 | ISGSGGST | 1155 | ARREWWYDDWYL | 1093 |
| | | | | | DY | |
| PALF04 | GFTFSSYA | 1082 | ISGSGGST | 1155 | ARREWWYDDWYL | 1093 |
| | | | | | DY | |
| PALF05 | GFTFSSYA | 1082 | ISGSGGST | 1155 | ARREWWYDDWYL | 1093 |
| | | | | | DY | |
| PALF06 | GFTFSSYA | 1082 | ISGSGGST | 1155 | ARREWWYDDWYL | 1093 |
| | | | | | DY | |
| PALF07 | GFTFSSYA | 1082 | ISGSGGST | 1155 | ARREWWYDDWYL | 1093 |
| | | | | | DY | |
| PALF08 | GFTFSSYA | 1082 | ISGSGGST | 1155 | ARREWWYDDWYL | 1093 |
| | | | | | DY | |
| PALF09 | GFTFSSYA | 1082 | ISGSGGST | 1155 | ARREWWYDDWYL | 1093 |
| | | | | | DY | |
| PALF12 | GFTFSSYA | 1082 | ISGSGGRA | 1157 | ARREWWYDDWYL | 1093 |
| | | | | | DY | |
| PALF13 | GFTFSSYA | 1082 | ISESGDVE | 1158 | ARREWWYDDWYL | 1093 |
| | | | | | DY | |
| PALF14 | GFTFSSYA | 1082 | ISESGDVE | 1158 | ARREWWYDDWYL | 1093 |
| | | | | | DY | |
| PALF15 | GFTFSSYA | 1082 | ISEHGHYT | 1159 | ARREWWYDDWYL | 1093 |
| | | | | | DY | |
| PALF16 | GFTFSSYA | 1082 | ISGSGHTA | 1160 | ARREWWYDDWYL | 1093 |
| | | | | | DY | |
| PALF17 | GFTFSSYA | 1082 | ISGSGRTH | 1161 | ARREWWYDDWYL | 1093 |
| | | | | | DY | |
| PALF18 | GFTFSSYA | 1082 | ISAEGGVR | 1162 | ARREWWYDDWYL | 1093 |
| | | | | | DY | |
| PALF19 | GFTFSSYA | 1082 | ISGSGGTT | 1163 | ARREWWYDDWYL | 1093 |
| | | | | | DY | |
| PALF20 | GFTFSSYA | 1082 | ISGSGATT | 1164 | ARREWWYDDWYL | 1093 |
| | | | | | DY | |

TABLE 151-1
AB3 family BCMA Binders-Light Chain CDR sequences according to Kabat numbering scheme

| | | SEQ ID | | SEQ ID | | SEQ ID |
|---|---|---|---|---|---|---|
| Binder | CDR-L1 | NO: | CDR-L2: | NO: | CDR-L3 | NO: |
| AB3 | TGTSSDVGGYNY | 1094 | DVSNRLR | 1165 | SSYTSSSALYV | 1173 |
| | VS | | | | | |
| PI-61 | TGTSSDVGGYNY | 1094 | DVSNRPS | 1166 | SSYTSSSTLYV | 1174 |
| | VS | | | | | |
| H2/L2-22 | TGTSSDVGGYNY | 1094 | EVSNRLS | 1167 | SSYTSSSTLYV | 1174 |
| | VS | | | | | |
| H2/L2-88 | TGTSSDVGGYNY | 1094 | EVSNRLR | 1168 | SSYTSSSALYV | 1173 |
| | VS | | | | | |
| H2/L2-36 | TGTSSDVGGYNY | 1094 | EVSNRLR | 1168 | SSYTSSSTLYV | 1174 |
| | VS | | | | | |
| H2/L2-34 | TGTSSDVGGYNY | 1094 | DVSNRPW | 1169 | SSYTSSSALYV | 1173 |
| | VS | | | | | |
| H2/L2-68 | TGTSSDVGGYNY | 1094 | DVSNRLS | 1170 | SSYTSSSTLYV | 1174 |
| | VS | | | | | |
| H2/L2-18 | TGTSSDVGGYNY | 1094 | DVSNRPW | 1169 | SSYTSSSTLYV | 1174 |
| | VS | | | | | |
| H2/L2-47 | TGTSSDVGGYNY | 1094 | DVSNRPW | 1169 | SSYTSSSTLYV | 1174 |
| | VS | | | | | |
| H2/L2-20 | TGTSSDVGGYNY | 1094 | DVSNRLR | 1165 | SSYTSSSALYV | 1173 |
| | VS | | | | | |
| H2/L2-80 | TGTSSDVGGYNY | 1094 | DVSNRAW | 1171 | SSYTSSSALYV | 1173 |
| | VS | | | | | |
| H2/L2-83 | TGTSSDVGGYNY | 1094 | EVSNRLR | 1168 | SSYTSSSTLYV | 1174 |
| | VS | | | | | |
| H3-1 | TGTSSDVGGYNY | 1094 | EVSNRLR | 1168 | SSYTSSSTLYV | 1174 |
| | VS | | | | | |
| H3-2 | TGTSSDVGGYNY | 1094 | EVSNRLR | 1168 | SSYTSSSTLYV | 1174 |
| | VS | | | | | |
| H3-3 | TGTSSDVGGYNY | 1094 | EVSNRLR | 1168 | SSYTSSSTLYV | 1174 |
| | VS | | | | | |
| H3-4 | TGTSSDVGGYNY | 1094 | EVSNRLR | 1168 | SSYTSSSALYV | 1173 |
| | VS | | | | | |
| H3-5 | TGTSSDVGGYNY | 1094 | EVSNRLS | 1167 | SSYTSSSTLYV | 1174 |
| | VS | | | | | |
| H3-6 | TGTSSDVGGYNY | 1094 | EVSNRLR | 1168 | SSYTSSSALYV | 1173 |
| | VS | | | | | |
| H3-7 | TGTSSDVGGYNY | 1094 | EVSNRLR | 1168 | SSYTSSSTLYV | 1174 |
| | VS | | | | | |
| H3-8 | TGTSSDVGGYNY | 1094 | EVSNRLR | 1168 | SSYTSSSTLYV | 1174 |
| | VS | | | | | |
| H3-9 | TGTSSDVGGYNY | 1094 | EVSNRLR | 1168 | SSYTSSSTLYV | 1174 |
| | VS | | | | | |
| H3-10 | TGTSSDVGGYNY | 1094 | EVSNRLR | 1168 | SSYTSSSTLYV | 1174 |
| | VS | | | | | |
| H3-11 | TGTSSDVGGYNY | 1094 | EVSNRLR | 1168 | SSYTSSSTLYV | 1174 |
| | VS | | | | | |
| H3-12 | TGTSSDVGGYNY | 1094 | EVSNRLR | 1168 | SSYTSSSTLYV | 1174 |
| | VS | | | | | |
| H3-13 | TGTSSDVGGYNY | 1094 | EVSNRLR | 1168 | SSYTSSSTLYV | 1174 |
| | VS | | | | | |
| H3-14 | TGTSSDVGGYNY | 1094 | EVSNRLS | 1167 | SSYTSSSALYV | 1173 |
| | VS | | | | | |
| H3-15 | TGTSSDVGGYNY | 1094 | EVSNRLG | 1172 | SSYTSSSALYV | 1173 |
| | VS | | | | | |

TABLE 151-2
AB3 family BCMA Binders-Heavy Chain CDR sequences according to Kabat numbering scheme

| | | SEQ ID | | SEQ ID | | SEQ ID |
|---|---|---|---|---|---|---|
| Binder | CDR-H1 | NO: | CDR-H2: | NO: | CDR-H3 | NO: |
| AB3 | SYGMH | 1107 | VISYTGSNKYYADSVK | 1175 | SGYALHDDYYGLDV | 1117 |
| | | | G | | | |
| PI-61 | SYGMH | 1107 | VISYDGSNKYYADSVK | 1176 | SGYALHDDYYGLDV | 1117 |
| | | | G | | | |
| H2/L2- | SYGMH | 1107 | VISYHGSNKYYADSVK | 1177 | SGYALHDDYYGLDV | 1117 |
| 22 | | | G | | | |
| H2/L2- | SYGMH | 1107 | VISYKGSNKYYADSVK | 1178 | SGYALHDDYYGLDV | 1117 |
| 88 | | | G | | | |
| H2/L2- | SYGMH | 1107 | VISYKGSNKYYADSVK | 1178 | SGYALHDDYYGLDV | 1117 |
| 36 | | | G | | | |
| H2/L2- | SYGMH | 1107 | VISYTGTKKYYADSVK | 1179 | SGYALHDDYYGLDV | 1117 |
| 34 | | | G | | | |
| H2/L2- | SYGMH | 1107 | VISYRGFNKYYADSVK | 1180 | SGYALHDDYYGQD | 1189 |
| 68 | | | G | | V | |
| H2/L2- | SYGMH | 1107 | VISYKGSHKYYADSVK | 1181 | SGYALHDDYYGLDV | 1117 |
| 18 | | | G | | | |
| H2/L2- | SYGMH | 1107 | VISYKGSNKYYADSVK | 1178 | SGYALHDDYYGLDV | 1117 |
| 47 | | | G | | | |
| H2/L2- | SYGMH | 1107 | VISYTGSNKYYADSVK | 1175 | SGYALHDDYYGLDV | 1117 |
| 20 | | | G | | | |
| H2/L2- | SYGMH | 1107 | VISYTGSNKYYADSVK | 1175 | SGYALHDDYYGLDV | 1117 |
| 80 | | | G | | | |
| H2/L2- | SYGMH | 1107 | VISYKGSNKYYADSVK | 1178 | SGYALHDDYYGLDV | 1117 |
| 83 | | | G | | | |
| H3-1 | SYGMH | 1107 | VISYDDAHKYYADSVK | 1182 | SGYALHDQYKPVD | 1190 |
| | | | G | | V | |
| H3-2 | SYGMH | 1107 | VISYNDLNKYYADSVK | 1183 | SGYALHDFQDPTDV | 1191 |
| | | | G | | | |
| H3-3 | SYGMH | 1107 | VISYSGSNKYYADSVK | 1184 | SGYALHDQYKPVD | 1190 |
| | | | G | | V | |
| H3-4 | SYGMH | 1107 | VISYDDAHKYYADSVK | 1182 | SGYALHDQYKPVD | 1190 |
| | | | G | | V | |
| H3-5 | SYGMH | 1107 | VISYTGANKYYADSVK | 1185 | SGYNLHDDYYGLD | 1192 |
| | | | G | | V | |
| H3-6 | SYGMH | 1107 | VISYDDAHKYYADSVK | 1182 | SGYALHDQYKPVD | 1190 |
| | | | G | | V | |
| H3-7 | SYGMH | 1107 | VISYTGSNKYYADSVK | 1175 | SGYEFHEDYYGLDV | 1193 |
| | | | G | | | |
| H3-8 | SYGMH | 1107 | VISYDDAHKYYADSVK | 1182 | SGYALHDQYKPVD | 1190 |
| | | | G | | V | |
| H3-9 | SYGMH | 1107 | VISYDDAHKYYADSVK | 1182 | SGYALHDQYKPVD | 1190 |
| | | | G | | V | |
| H3-10 | SYGMH | 1107 | VISYNDLNKYYADSVK | 1183 | SGYEFQGDYYGLD | 1194 |
| | | | G | | V | |
| H3-11 | SYGMH | 1107 | VISYNDANKYYADSVK | 1186 | SGYELRDDYYGLDV | 1195 |
| | | | G | | | |
| H3-12 | SYGMH | 1107 | VISYDESNKYYADSVK | 1187 | SGYEVDQDYYGLD | 1196 |
| | | | G | | V | |
| H3-13 | SYGMH | 1107 | VISYDDAHKYYADSVK | 1182 | SGYALHDQYKPVD | 1190 |
| | | | G | | V | |
| H3-14 | SYGMH | 1107 | VISYDDAHKYYADSVK | 1182 | SGYALHDQYKPVD | 1190 |
| | | | G | | V | |
| H3-15 | SYGMH | 1107 | VISYDDAHKYYADSVK | 1188 | SGYAYDGDYYGLD | 1197 |
| | | | G | | V | |

TABLE 15J-1
AB3 family BCMA Binders-Light Chain CDR sequences according to Chothia
numbering scheme

| | | SEQ ID | | SEQ ID | | SEQ ID |
|---|---|---|---|---|---|---|
| Binder | CDR-L1 | NO: | CDR-L2: | NO: | CDR-L3 | NO: |
| AB3 | TSSDVGGYNY | 1095 | DVS | 1099 | YTSSSALY | 1198 |
| PI-61 | TSSDVGGYNY | 1095 | DVS | 1099 | YTSSSTLY | 1199 |
| H2/L2-22 | TSSDVGGYNY | 1095 | EVS | 580 | YTSSSTLY | 1199 |
| H2/L2-88 | TSSDVGGYNY | 1095 | EVS | 580 | YTSSSALY | 1198 |
| H2/L2-36 | TSSDVGGYNY | 1095 | EVS | 580 | YTSSSTLY | 1199 |
| H2/L2-34 | TSSDVGGYNY | 1095 | DVS | 1099 | YTSSSALY | 1198 |
| H2/L2-68 | TSSDVGGYNY | 1095 | DVS | 1099 | YTSSSTLY | 1199 |
| H2/L2-18 | TSSDVGGYNY | 1095 | DVS | 1099 | YTSSSTLY | 1199 |
| H2/L2-47 | TSSDVGGYNY | 1095 | DVS | 1099 | YTSSSTLY | 1199 |
| H2/L2-20 | TSSDVGGYNY | 1095 | DVS | 1099 | YTSSSALY | 1198 |
| H2/L2-80 | TSSDVGGYNY | 1095 | DVS | 1099 | YTSSSALY | 1198 |
| H2/L2-83 | TSSDVGGYNY | 1095 | EVS | 580 | YTSSSTLY | 1199 |
| H3-1 | TSSDVGGYNY | 1095 | EVS | 580 | YTSSSTLY | 1199 |
| H3-2 | TSSDVGGYNY | 1095 | EVS | 580 | YTSSSTLY | 1199 |
| H3-3 | TSSDVGGYNY | 1095 | EVS | 580 | YTSSSTLY | 1199 |
| H3-4 | TSSDVGGYNY | 1095 | EVS | 580 | YTSSSALY | 1198 |
| H3-5 | TSSDVGGYNY | 1095 | EVS | 580 | YTSSSTLY | 1199 |
| H3-6 | TSSDVGGYNY | 1095 | EVS | 580 | YTSSSALY | 1198 |
| H3-7 | TSSDVGGYNY | 1095 | EVS | 580 | YTSSSTLY | 1199 |
| H3-8 | TSSDVGGYNY | 1095 | EVS | 580 | YTSSSTLY | 1199 |
| H3-9 | TSSDVGGYNY | 1095 | EVS | 580 | YTSSSTLY | 1199 |
| H3-10 | TSSDVGGYNY | 1095 | EVS | 580 | YTSSSTLY | 1199 |
| H3-11 | TSSDVGGYNY | 1095 | EVS | 580 | YTSSSTLY | 1199 |
| H3-12 | TSSDVGGYNY | 1095 | EVS | 580 | YTSSSTLY | 1199 |
| H3-13 | TSSDVGGYNY | 1095 | EVS | 580 | YTSSSTLY | 1199 |
| H3-14 | TSSDVGGYNY | 1095 | EVS | 580 | YTSSSALY | 1198 |
| H3-15 | TSSDVGGYNY | 1095 | EVS | 580 | YTSSSALY | 1198 |

TABLE 15J-2
AB3 family BCMA Binders-Heavy Chain CDR sequences according to Chothia
numbering scheme

| | | SEQ ID | | SEQ ID | | SEQ ID |
|---|---|---|---|---|---|---|
| Binder | CDR-H1 | NO: | CDR-H2: | NO: | CDR-H3 | NO: |
| AB3 | GFTVSSY | 1200 | SYTGSN | 1202 | SGYALHDDYYGLD | 1117 |
| | | | | | V | |
| PI-61 | GFTFSSY | 690 | SYDGSN | 1203 | SGYALHDDYYGLD | 1117 |
| | | | | | V | |
| H2/L2-22 | GFTFSSY | 690 | SYHGSN | 1204 | SGYALHDDYYGLD | 1117 |
| | | | | | V | |
| H2/L2-88 | GFTFSSY | 690 | SYKGSN | 1205 | SGYALHDDYYGLD | 1117 |
| | | | | | V | |
| H2/L2-36 | GFTFSSY | 690 | SYKGSN | 1205 | SGYALHDDYYGLD | 1117 |
| | | | | | V | |
| H2/L2-34 | GFTFSSY | 690 | SYTGTK | 1206 | SGYALHDDYYGLD | 1117 |
| | | | | | V | |
| H2/L2-68 | GFTFSSY | 690 | SYRGFN | 1207 | SGYALHDDYYGQ | 1189 |
| | | | | | DV | |
| H2/L2-18 | GFTFSSY | 690 | SYKGSH | 1208 | SGYALHDDYYGLD | 1117 |
| | | | | | V | |
| H2/L2-47 | GFTFSSY | 690 | SYKGSN | 1205 | SGYALHDDYYGLD | 1117 |
| | | | | | V | |
| H2/L2-20 | GFTVSSY | 1200 | SYTGSN | 1202 | SGYALHDDYYGLD | 1117 |
| | | | | | V | |
| H2/L2-80 | GFTFSSY | 690 | SYTGSN | 1202 | SGYALHDDYYGLD | 1117 |
| | | | | | V | |
| H2/L2-83 | GFTFSSY | 690 | SYKGSN | 1205 | SGYALHDDYYGLD | 1117 |
| | | | | | V | |
| H3-1 | GFTFSSY | 690 | SYDDAH | 1209 | SGYALHDQYKPVD | 1190 |
| | | | | | V | |
| H3-2 | GFTFSSY | 690 | SYNDLN | 1210 | SGYALHDFQDPTD | 1191 |
| | | | | | V | |
| H3-3 | GFTVSSY | 1200 | SYSGSN | 1211 | SGYALHDQYKPVD | 1190 |
| | | | | | V | |
| H3-4 | GFTFSSY | 690 | SYDDAH | 1209 | SGYALHDQYKPVD | 1190 |
| | | | | | V | |
| H3-5 | GFTFSSY | 690 | SYTGAN | 1212 | SGYNLHDDYYGLD | 1192 |
| | | | | | V | |
| H3-6 | GFTFSSY | 690 | SYDDAH | 1209 | SGYALHDQYKPVD | 1190 |
| | | | | | V | |
| H3-7 | GFTLSSY | 1201 | SYTGSN | 1202 | SGYEFHEDYYGLD | 1193 |
| | | | | | V | |
| H3-8 | GFTFSSY | 690 | SYDDAH | 1209 | SGYALHDQYKPVD | 1190 |
| | | | | | V | |
| H3-9 | GFTFSSY | 690 | SYDDAH | 1209 | SGYALHDQYKPVD | 1190 |
| | | | | | V | |
| H3-10 | GFTFSSY | 690 | SYNDLN | 1210 | SGYEFQGDYYGL | 1194 |
| | | | | | DV | |
| H3-11 | GFTFSSY | 690 | SYNDAN | 1213 | SGYELRDDYYGLD | 1195 |
| | | | | | V | |
| H3-12 | GFTFSSY | 690 | SYDESN | 1214 | SGYEVDQDYYGL | 1196 |
| | | | | | DV | |
| H3-13 | GFTFSSY | 690 | SYDDAH | 1209 | SGYALHDQYKPVD | 1190 |
| | | | | | V | |
| H3-14 | GFTFSSY | 690 | SYDDAH | 1209 | SGYALHDQYKPVD | 1190 |
| | | | | | V | |
| H3-15 | GFTVSSY | 1200 | SYDDAN | 1215 | SGYAYDGDYYGL | 1197 |
| | | | | | DV | |

TABLE 15K-1(a)
AB3 family BCMA Binders-CDR-L1 and CDR-L3 sequences according to IMGT
numbering scheme and CDR-L2 expanded sequences

| | | SEQ ID | | SEQ ID | | SEQ ID |
|---|---|---|---|---|---|---|
| Binder | CDR-L1 | NO: | CDR-L2: | NO: | CDR-L3 | NO: |
| AB3 | SSDVGGYNY | 1096 | DVSNRLRGVS | 1216 | SSYTSSSALYV | 1173 |
| PI-61 | SSDVGGYNY | 1096 | DVSNRPSGVS | 1217 | SSYTSSSTLYV | 1174 |
| H2/L2-22 | SSDVGGYNY | 1096 | EVSNRLSGVS | 1218 | SSYTSSSTLYV | 1174 |
| H2/L2-88 | SSDVGGYNY | 1096 | EVSNRLRGVS | 1219 | SSYTSSSALYV | 1173 |
| H2/L2-36 | SSDVGGYNY | 1096 | EVSNRLRGVS | 1219 | SSYTSSSTLYV | 1174 |
| H2/L2-34 | SSDVGGYNY | 1096 | DVSNRPWGVS | 1220 | SSYTSSSALYV | 1173 |
| H2/L2-68 | SSDVGGYNY | 1096 | DVSNRLSGVS | 1221 | SSYTSSSTLYV | 1174 |
| H2/L2-18 | SSDVGGYNY | 1096 | DVSNRPWGVS | 1220 | SSYTSSSTLYV | 1174 |
| H2/L2-47 | SSDVGGYNY | 1096 | DVSNRPWGVS | 1220 | SSYTSSSTLYV | 1174 |
| H2/L2-20 | SSDVGGYNY | 1096 | DVSNRLRGVS | 1216 | SSYTSSSALYV | 1173 |
| H2/L2-80 | SSDVGGYNY | 1096 | DVSNRAWGVS | 1222 | SSYTSSSALYV | 1173 |
| H2/L2-83 | SSDVGGYNY | 1096 | EVSNRLRGVS | 1219 | SSYTSSSTLYV | 1174 |
| H3-1 | SSDVGGYNY | 1096 | EVSNRLRGVS | 1219 | SSYTSSSTLYV | 1174 |
| H3-2 | SSDVGGYNY | 1096 | EVSNRLRGVS | 1219 | SSYTSSSTLYV | 1174 |
| H3-3 | SSDVGGYNY | 1096 | EVSNRLRGVS | 1219 | SSYTSSSTLYV | 1174 |
| H3-4 | SSDVGGYNY | 1096 | EVSNRLRGVS | 1219 | SSYTSSSALYV | 1173 |
| H3-5 | SSDVGGYNY | 1096 | EVSNRLSGVS | 1218 | SSYTSSSTLYV | 1174 |
| H3-6 | SSDVGGYNY | 1096 | EVSNRLRGVS | 1219 | SSYTSSSALYV | 1173 |
| H3-7 | SSDVGGYNY | 1096 | EVSNRLRGVS | 1219 | SSYTSSSTLYV | 1174 |
| H3-8 | SSDVGGYNY | 1096 | EVSNRLRGVS | 1219 | SSYTSSSTLYV | 1174 |
| H3-9 | SSDVGGYNY | 1096 | EVSNRLRGVS | 1219 | SSYTSSSTLYV | 1174 |
| H3-10 | SSDVGGYNY | 1096 | EVSNRLRGVS | 1219 | SSYTSSSTLYV | 1174 |
| H3-11 | SSDVGGYNY | 1096 | EVSNRLRGVS | 1219 | SSYTSSSTLYV | 1174 |
| H3-12 | SSDVGGYNY | 1096 | EVSNRLRGVS | 1219 | SSYTSSSTLYV | 1174 |
| H3-13 | SSDVGGYNY | 1096 | EVSNRLRGVS | 1219 | SSYTSSSTLYV | 1174 |
| H3-14 | SSDVGGYNY | 1096 | EVSNRLSGVS | 1218 | SSYTSSSALYV | 1173 |
| H3-15 | SSDVGGYNY | 1096 | EVSNRLGGVS | 1223 | SSYTSSSALYV | 1173 |

TABLE 15K-1(b)
AB3 family BCMA Binders-Light Chain CDR sequences according to IMGT
numbering scheme

| | | SEQ ID | | SEQ ID | | SEQ ID |
|---|---|---|---|---|---|---|
| Binder | CDR-L1 | NO: | CDR-L2: | NO: | CDR-L3 | NO: |
| AB3 | SSDVGGYNY | 1096 | DVS | 1099 | SSYTSSSALYV | 1173 |
| PI-61 | SSDVGGYNY | 1096 | DVS | 1099 | SSYTSSSTLYV | 1174 |
| H2/L2-22 | SSDVGGYNY | 1096 | EVS | 580 | SSYTSSSTLYV | 1174 |
| H2/L2-88 | SSDVGGYNY | 1096 | EVS | 580 | SSYTSSSALYV | 1173 |
| H2/L2-36 | SSDVGGYNY | 1096 | EVS | 580 | SSYTSSSTLYV | 1174 |
| H2/L2-34 | SSDVGGYNY | 1096 | DVS | 1099 | SSYTSSSALYV | 1173 |
| H2/L2-68 | SSDVGGYNY | 1096 | DVS | 1099 | SSYTSSSTLYV | 1174 |
| H2/L2-18 | SSDVGGYNY | 1096 | DVS | 1099 | SSYTSSSTLYV | 1174 |
| H2/L2-47 | SSDVGGYNY | 1096 | DVS | 1099 | SSYTSSSTLYV | 1174 |
| H2/L2-20 | SSDVGGYNY | 1096 | DVS | 1099 | SSYTSSSALYV | 1173 |
| H2/L2-80 | SSDVGGYNY | 1096 | DVS | 1099 | SSYTSSSALYV | 1173 |
| H2/L2-83 | SSDVGGYNY | 1096 | EVS | 580 | SSYTSSSTLYV | 1174 |
| H3-1 | SSDVGGYNY | 1096 | EVS | 580 | SSYTSSSTLYV | 1174 |
| H3-2 | SSDVGGYNY | 1096 | EVS | 580 | SSYTSSSTLYV | 1174 |
| H3-3 | SSDVGGYNY | 1096 | EVS | 580 | SSYTSSSTLYV | 1174 |
| H3-4 | SSDVGGYNY | 1096 | EVS | 580 | SSYTSSSALYV | 1173 |
| H3-5 | SSDVGGYNY | 1096 | EVS | 580 | SSYTSSSTLYV | 1174 |
| H3-6 | SSDVGGYNY | 1096 | EVS | 580 | SSYTSSSALYV | 1173 |
| H3-7 | SSDVGGYNY | 1096 | EVS | 580 | SSYTSSSTLYV | 1174 |
| H3-8 | SSDVGGYNY | 1096 | EVS | 580 | SSYTSSSTLYV | 1174 |
| H3-9 | SSDVGGYNY | 1096 | EVS | 580 | SSYTSSSTLYV | 1174 |
| H3-10 | SSDVGGYNY | 1096 | EVS | 580 | SSYTSSSTLYV | 1174 |
| H3-11 | SSDVGGYNY | 1096 | EVS | 580 | SSYTSSSTLYV | 1174 |
| H3-12 | SSDVGGYNY | 1096 | EVS | 580 | SSYTSSSTLYV | 1174 |
| H3-13 | SSDVGGYNY | 1096 | EVS | 580 | SSYTSSSTLYV | 1174 |
| H3-14 | SSDVGGYNY | 1096 | EVS | 580 | SSYTSSSALYV | 1173 |
| H3-15 | SSDVGGYNY | 1096 | EVS | 580 | SSYTSSSALYV | 1173 |

TABLE 15K-2
AB3 family BCMA Binders-Heavy Chain CDR sequences according to IMGT
numbering scheme

| | | SEQ ID | | SEQ ID | | SEQ ID |
|---|---|---|---|---|---|---|
| Binder | CDR-H1 | NO: | CDR-H2: | NO: | CDR-H3 | NO: |
| AB3 | GFTVSSYG | 1224 | ISYTGSNK | 1227 | GGSGYALHDDYYGL | 1119 |
| | | | | | DV | |
| PI-61 | GFTFSSYG | 1225 | ISYDGSNK | 1228 | GGSGYALHDDYYGL | 1119 |
| | | | | | DV | |
| H2/L2-22 | GFTFSSYG | 1225 | ISYHGSNK | 1229 | GGSGYALHDDYYGL | 1119 |
| | | | | | DV | |
| H2/L2-88 | GFTFSSYG | 1225 | ISYKGSNK | 1230 | GGSGYALHDDYYGL | 1119 |
| | | | | | DV | |
| H2/L2-36 | GFTFSSYG | 1225 | ISYKGSNK | 1230 | GGSGYALHDDYYGL | 1119 |
| | | | | | DV | |
| H2/L2-34 | GFTFSSYG | 1225 | ISYTGTKK | 1231 | GGSGYALHDDYYGL | 1119 |
| | | | | | DV | |
| H2/L2-68 | GFTFSSYG | 1225 | ISYRGFNK | 1232 | GGSGYALHDDYYGQ | 1241 |
| | | | | | DV | |
| H2/L2-18 | GFTFSSYG | 1225 | ISYKGSHK | 1233 | GGSGYALHDDYYGL | 1119 |
| | | | | | DV | |
| H2/L2-47 | GFTFSSYG | 1225 | ISYKGSNK | 1230 | GGSGYALHDDYYGL | 1119 |
| | | | | | DV | |
| H2/L2-20 | GFTVSSYG | 1224 | ISYTGSNK | 1227 | GGSGYALHDDYYGL | 1119 |
| | | | | | DV | |
| H2/L2-80 | GFTFSSYG | 1225 | ISYTGSNK | 1227 | GGSGYALHDDYYGL | 1119 |
| | | | | | DV | |
| H2/L2-83 | GFTFSSYG | 1225 | ISYKGSNK | 1230 | GGSGYALHDDYYGL | 1119 |
| | | | | | DV | |
| H3-1 | GFTFSSYG | 1225 | ISYDDAHK | 1234 | GGSGYALHDQYKPV | 1242 |
| | | | | | DV | |
| H3-2 | GFTFSSYG | 1225 | ISYNDLNK | 1235 | GGSGYALHDFQDPT | 1243 |
| | | | | | DV | |
| H3-3 | GFTVSSYG | 1224 | ISYSGSNK | 1236 | GGSGYALHDQYKPV | 1242 |
| | | | | | DV | |
| H3-4 | GFTFSSYG | 1225 | ISYDDAHK | 1234 | GGSGYALHDQYKPV | 1242 |
| | | | | | DV | |
| H3-5 | GFTFSSYG | 1225 | ISYTGANK | 1237 | GGSGYNLHDDYYGL | 1244 |
| | | | | | DV | |
| H3-6 | GFTFSSYG | 1225 | ISYDDAHK | 1234 | GGSGYALHDQYKPV | 1242 |
| | | | | | DV | |
| H3-7 | GFTLSSYG | 1226 | ISYTGSNK | 1227 | GGSGYEFHEDYYGL | 1245 |
| | | | | | DV | |
| H3-8 | GFTFSSYG | 1225 | ISYDDAHK | 1234 | GGSGYALHDQYKPV | 1242 |
| | | | | | DV | |
| H3-9 | GFTFSSYG | 1225 | ISYDDAHK | 1234 | GGSGYALHDQYKPV | 1242 |
| | | | | | DV | |
| H3-10 | GFTFSSYG | 1225 | ISYNDLNK | 1235 | GGSGYEFQGDYYGL | 1246 |
| | | | | | DV | |
| H3-11 | GFTFSSYG | 1225 | ISYNDANK | 1238 | GGSGYELRDDYYGL | 1247 |
| | | | | | DV | |
| H3-12 | GFTFSSYG | 1225 | ISYDESNK | 1239 | GGSGYEVDQDYYGL | 1248 |
| | | | | | DV | |
| H3-13 | GFTFSSYG | 1225 | ISYDDAHK | 1234 | GGSGYALHDQYKPV | 1242 |
| | | | | | DV | |
| H3-14 | GFTFSSYG | 1225 | ISYDDAHK | 1234 | GGSGYALHDQYKPV | 1242 |
| | | | | | DV | |
| H3-15 | GFTVSSYG | 1224 | ISYDDANK | 1240 | GGSGYAYDGDYYGL | 1249 |
| | | | | | DV | |

TABLE 15L-1
AB3 family BCMA Binders-Light Chain CDR sequences according to combination
of Kabat and Chothia numbering schemes

| | | SEQ ID | | SEQ ID | | SEQ ID |
|---|---|---|---|---|---|---|
| Binder | CDR-L1 | NO: | CDR-L2: | NO: | CDR-L3 | NO: |
| AB3 | TGTSSDVGGYN | 1094 | DVSNRLR | 1165 | SSYTSSSALYV | 1173 |
| | YVS | | | | | |
| PI-61 | TGTSSDVGGYN | 1094 | DVSNRPS | 1166 | SSYTSSSTLYV | 1174 |
| | YVS | | | | | |
| H2/L2-22 | TGTSSDVGGYN | 1094 | EVSNRLS | 1167 | SSYTSSSTLYV | 1174 |
| | YVS | | | | | |
| H2/L2-88 | TGTSSDVGGYN | 1094 | EVSNRLR | 1168 | SSYTSSSALYV | 1173 |
| | YVS | | | | | |
| H2/L2-36 | TGTSSDVGGYN | 1094 | EVSNRLR | 1168 | SSYTSSSTLYV | 1174 |
| | YVS | | | | | |
| H2/L2-34 | TGTSSDVGGYN | 1094 | DVSNRPW | 1169 | SSYTSSSALYV | 1173 |
| | YVS | | | | | |
| H2/L2-68 | TGTSSDVGGYN | 1094 | DVSNRLS | 1170 | SSYTSSSTLYV | 1174 |
| | YVS | | | | | |
| H2/L2-18 | TGTSSDVGGYN | 1094 | DVSNRPW | 1169 | SSYTSSSTLYV | 1174 |
| | YVS | | | | | |
| H2/L2-47 | TGTSSDVGGYN | 1094 | DVSNRPW | 1169 | SSYTSSSTLYV | 1174 |
| | YVS | | | | | |
| H2/L2-20 | TGTSSDVGGYN | 1094 | DVSNRLR | 1165 | SSYTSSSALYV | 1173 |
| | YVS | | | | | |
| H2/L2-80 | TGTSSDVGGYN | 1094 | DVSNRAW | 1171 | SSYTSSSALYV | 1173 |
| | YVS | | | | | |
| H2/L2-83 | TGTSSDVGGYN | 1094 | EVSNRLR | 1168 | SSYTSSSTLYV | 1174 |
| | YVS | | | | | |
| H3-1 | TGTSSDVGGYN | 1094 | EVSNRLR | 1168 | SSYTSSSTLYV | 1174 |
| | YVS | | | | | |
| H3-2 | TGTSSDVGGYN | 1094 | EVSNRLR | 1168 | SSYTSSSTLYV | 1174 |
| | YVS | | | | | |
| H3-3 | TGTSSDVGGYN | 1094 | EVSNRLR | 1168 | SSYTSSSTLYV | 1174 |
| | YVS | | | | | |
| H3-4 | TGTSSDVGGYN | 1094 | EVSNRLR | 1168 | SSYTSSSALYV | 1173 |
| | YVS | | | | | |
| H3-5 | TGTSSDVGGYN | 1094 | EVSNRLS | 1167 | SSYTSSSTLYV | 1174 |
| | YVS | | | | | |
| H3-6 | TGTSSDVGGYN | 1094 | EVSNRLR | 1168 | SSYTSSSALYV | 1173 |
| | YVS | | | | | |
| H3-7 | TGTSSDVGGYN | 1094 | EVSNRLR | 1168 | SSYTSSSTLYV | 1174 |
| | YVS | | | | | |
| H3-8 | TGTSSDVGGYN | 1094 | EVSNRLR | 1168 | SSYTSSSTLYV | 1174 |
| | YVS | | | | | |
| H3-9 | TGTSSDVGGYN | 1094 | EVSNRLR | 1168 | SSYTSSSTLYV | 1174 |
| | YVS | | | | | |
| H3-10 | TGTSSDVGGYN | 1094 | EVSNRLR | 1168 | SSYTSSSTLYV | 1174 |
| | YVS | | | | | |
| H3-11 | TGTSSDVGGYN | 1094 | EVSNRLR | 1168 | SSYTSSSTLYV | 1174 |
| | YVS | | | | | |
| H3-12 | TGTSSDVGGYN | 1094 | EVSNRLR | 1168 | SSYTSSSTLYV | 1174 |
| | YVS | | | | | |
| H3-13 | TGTSSDVGGYN | 1094 | EVSNRLR | 1168 | SSYTSSSTLYV | 1174 |
| | YVS | | | | | |
| H3-14 | TGTSSDVGGYN | 1094 | EVSNRLS | 1167 | SSYTSSSALYV | 1173 |
| | YVS | | | | | |
| H3-15 | TGTSSDVGGYN | 1094 | EVSNRLG | 1172 | SSYTSSSALYV | 1173 |
| | YVS | | | | | |

TABLE 15L-2
AB3 family BCMA Binders-Heavy Chain CDR sequences according to combination of
Kabat and Chothia numbering schemes

| | | SEQ ID | | SEQ ID | | SEQ ID |
|---|---|---|---|---|---|---|
| Binder | CDR-H1 | NO: | CDR-H2: | NO: | CDR-H3 | NO: |
| AB3 | GFTVSSYGMH | 1250 | VISYTGSNKYYA | 1175 | SGYALHDDYYGLD | 1117 |
| | | | DSVKG | | V | |
| PI-61 | GFTFSSYGMH | 1251 | VISYDGSNKYYA | 1176 | SGYALHDDYYGLD | 1117 |
| | | | DSVKG | | V | |
| H2/L2-22 | GFTFSSYGMH | 1251 | VISYHGSNKYYA | 1177 | SGYALHDDYYGLD | 1117 |
| | | | DSVKG | | V | |
| H2/L2-88 | GFTFSSYGMH | 1251 | VISYKGSNKYYA | 1178 | SGYALHDDYYGLD | 1117 |
| | | | DSVKG | | V | |
| H2/L2-36 | GFTFSSYGMH | 1251 | VISYKGSNKYYA | 1178 | SGYALHDDYYGLD | 1117 |
| | | | DSVKG | | V | |
| H2/L2-34 | GFTFSSYGMH | 1251 | VISYTGTKKYYAD | 1179 | SGYALHDDYYGLD | 1117 |
| | | | SVKG | | V | |
| H2/L2-68 | GFTFSSYGMH | 1251 | VISYRGFNKYYA | 1180 | SGYALHDDYYGQD | 1189 |
| | | | DSVKG | | V | |
| H2/L2-18 | GFTFSSYGMH | 1251 | VISYKGSHKYYA | 1181 | SGYALHDDYYGLD | 1117 |
| | | | DSVKG | | V | |
| H2/L2-47 | GFTFSSYGMH | 1251 | VISYKGSNKYYA | 1178 | SGYALHDDYYGLD | 1117 |
| | | | DSVKG | | V | |
| H2/L2-20 | GFTVSSYGMH | 1250 | VISYTGSNKYYA | 1175 | SGYALHDDYYGLD | 1117 |
| | | | DSVKG | | V | |
| H2/L2-80 | GFTFSSYGMH | 1251 | VISYTGSNKYYA | 1175 | SGYALHDDYYGLD | 1117 |
| | | | DSVKG | | V | |
| H2/L2-83 | GFTFSSYGMH | 1251 | VISYKGSNKYYA | 1178 | SGYALHDDYYGLD | 1117 |
| | | | DSVKG | | V | |
| H3-1 | GFTFSSYGMH | 1251 | VISYDDAHKYYA | 1182 | SGYALHDQYKPVD | 1190 |
| | | | DSVKG | | V | |
| H3-2 | GFTFSSYGMH | 1251 | VISYNDLNKYYAD | 1183 | SGYALHDFQDPTD | 1191 |
| | | | SVKG | | V | |
| H3-3 | GFTVSSYGMH | 1250 | VISYSGSNKYYA | 1184 | SGYALHDQYKPVD | 1190 |
| | | | DSVKG | | V | |
| H3-4 | GFTFSSYGMH | 1251 | VISYDDAHKYYA | 1182 | SGYALHDQYKPVD | 1190 |
| | | | DSVKG | | V | |
| H3-5 | GFTFSSYGMH | 1251 | VISYTGANKYYA | 1185 | SGYNLHDDYYGLD | 1192 |
| | | | DSVKG | | V | |
| H3-6 | GFTFSSYGMH | 1251 | VISYDDAHKYYA | 1182 | SGYALHDQYKPVD | 1190 |
| | | | DSVKG | | V | |
| H3-7 | GFTLSSYGMH | 1252 | VISYTGSNKYYA | 1175 | SGYEFHEDYYGLD | 1193 |
| | | | DSVKG | | V | |
| H3-8 | GFTFSSYGMH | 1251 | VISYDDAHKYYA | 1182 | SGYALHDQYKPVD | 1190 |
| | | | DSVKG | | V | |
| H3-9 | GFTFSSYGMH | 1251 | VISYDDAHKYYA | 1182 | SGYALHDQYKPVD | 1190 |
| | | | DSVKG | | V | |
| H3-10 | GFTFSSYGMH | 1251 | VISYNDLNKYYAD | 1183 | SGYEFQGDYYGLD | 1194 |
| | | | SVKG | | V | |
| H3-11 | GFTFSSYGMH | 1251 | VISYNDANKYYA | 1186 | SGYELRDDYYGLD | 1195 |
| | | | DSVKG | | V | |
| H3-12 | GFTFSSYGMH | 1251 | VISYDESNKYYA | 1187 | SGYEVDQDYYGLD | 1196 |
| | | | DSVKG | | V | |
| H3-13 | GFTFSSYGMH | 1251 | VISYDDAHKYYA | 1182 | SGYALHDQYKPVD | 1190 |
| | | | DSVKG | | V | |
| H3-14 | GFTFSSYGMH | 1251 | VISYDDAHKYYA | 1182 | SGYALHDQYKPVD | 1190 |
| | | | DSVKG | | V | |
| H3-15 | GFTVSSYGMH | 1250 | VISYDDANKYYA | 1188 | SGYAYDGDYYGLD | 1197 |
| | | | DSVKG | | V | |

TABLE 15M-1
AB3 family BCMA Binders-Light Chain CDR sequences according to combination
of Kabat and IMGT numbering schemes

| | | SEQ ID | | SEQ ID | | SEQ ID |
|---|---|---|---|---|---|---|
| Binder | CDR-L1 | NO: | CDR-L2: | NO: | CDR-L3 | NO: |
| AB3 | TGTSSDVGGYN | 1094 | DVSNRLR | 1165 | SSYTSSSALYV | 1173 |
| | YVS | | | | | |
| PI-61 | TGTSSDVGGYN | 1094 | DVSNRPS | 1166 | SSYTSSSTLYV | 1174 |
| | YVS | | | | | |
| H2/L2-22 | TGTSSDVGGYN | 1094 | EVSNRLS | 1167 | SSYTSSSTLYV | 1174 |
| | YVS | | | | | |
| H2/L2-88 | TGTSSDVGGYN | 1094 | EVSNRLR | 1168 | SSYTSSSALYV | 1173 |
| | YVS | | | | | |
| H2/L2-36 | TGTSSDVGGYN | 1094 | EVSNRLR | 1168 | SSYTSSSTLYV | 1174 |
| | YVS | | | | | |
| H2/L2-34 | TGTSSDVGGYN | 1094 | DVSNRPW | 1169 | SSYTSSSALYV | 1173 |
| | YVS | | | | | |
| H2/L2-68 | TGTSSDVGGYN | 1094 | DVSNRLS | 1170 | SSYTSSSTLYV | 1174 |
| | YVS | | | | | |
| H2/L2-18 | TGTSSDVGGYN | 1094 | DVSNRPW | 1169 | SSYTSSSTLYV | 1174 |
| | YVS | | | | | |
| H2/L2-47 | TGTSSDVGGYN | 1094 | DVSNRPW | 1169 | SSYTSSSTLYV | 1174 |
| | YVS | | | | | |
| H2/L2-20 | TGTSSDVGGYN | 1094 | DVSNRLR | 1165 | SSYTSSSALYV | 1173 |
| | YVS | | | | | |
| H2/L2-80 | TGTSSDVGGYN | 1094 | DVSNRAW | 1171 | SSYTSSSALYV | 1173 |
| | YVS | | | | | |
| H2/L2-83 | TGTSSDVGGYN | 1094 | EVSNRLR | 1168 | SSYTSSSTLYV | 1174 |
| | YVS | | | | | |
| H3-1 | TGTSSDVGGYN | 1094 | EVSNRLR | 1168 | SSYTSSSTLYV | 1174 |
| | YVS | | | | | |
| H3-2 | TGTSSDVGGYN | 1094 | EVSNRLR | 1168 | SSYTSSSTLYV | 1174 |
| | YVS | | | | | |
| H3-3 | TGTSSDVGGYN | 1094 | EVSNRLR | 1168 | SSYTSSSTLYV | 1174 |
| | YVS | | | | | |
| H3-4 | TGTSSDVGGYN | 1094 | EVSNRLR | 1168 | SSYTSSSALYV | 1173 |
| | YVS | | | | | |
| H3-5 | TGTSSDVGGYN | 1094 | EVSNRLS | 1167 | SSYTSSSTLYV | 1174 |
| | YVS | | | | | |
| H3-6 | TGTSSDVGGYN | 1094 | EVSNRLR | 1168 | SSYTSSSALYV | 1173 |
| | YVS | | | | | |
| H3-7 | TGTSSDVGGYN | 1094 | EVSNRLR | 1168 | SSYTSSSTLYV | 1174 |
| | YVS | | | | | |
| H3-8 | TGTSSDVGGYN | 1094 | EVSNRLR | 1168 | SSYTSSSTLYV | 1174 |
| | YVS | | | | | |
| H3-9 | TGTSSDVGGYN | 1094 | EVSNRLR | 1168 | SSYTSSSTLYV | 1174 |
| | YVS | | | | | |
| H3-10 | TGTSSDVGGYN | 1094 | EVSNRLR | 1168 | SSYTSSSTLYV | 1174 |
| | YVS | | | | | |
| H3-11 | TGTSSDVGGYN | 1094 | EVSNRLR | 1168 | SSYTSSSTLYV | 1174 |
| | YVS | | | | | |
| H3-12 | TGTSSDVGGYN | 1094 | EVSNRLR | 1168 | SSYTSSSTLYV | 1174 |
| | YVS | | | | | |
| H3-13 | TGTSSDVGGYN | 1094 | EVSNRLR | 1168 | SSYTSSSTLYV | 1174 |
| | YVS | | | | | |
| H3-14 | TGTSSDVGGYN | 1094 | EVSNRLS | 1167 | SSYTSSSALYV | 1173 |
| | YVS | | | | | |
| H3-15 | TGTSSDVGGYN | 1094 | EVSNRLG | 1172 | SSYTSSSALYV | 1173 |
| | YVS | | | | | |

TABLE 15M-2
AB3 family BCMA Binders-Heavy Chain CDR sequences according to combination of
Kabat and IMGT numbering schemes

| | | SEQ ID | | SEQ ID | | SEQ ID |
|---|---|---|---|---|---|---|
| Binder | CDR-H1 | NO: | CDR-H2: | NO: | CDR-H3 | NO: |
| AB3 | GFTVSSYGMH | 1250 | VISYTGSNKYYAD | 1175 | GGSGYALHDDYY | 1119 |
| | | | SVKG | | GLDV | |
| PI-61 | GFTFSSYGMH | 1251 | VISYDGSNKYYA | 1176 | GGSGYALHDDYY | 1119 |
| | | | DSVKG | | GLDV | |
| H2/L2-22 | GFTFSSYGMH | 1251 | VISYHGSNKYYA | 1177 | GGSGYALHDDYY | 1119 |
| | | | DSVKG | | GLDV | |
| H2/L2-88 | GFTFSSYGMH | 1251 | VISYKGSNKYYAD | 1178 | GGSGYALHDDYY | 1119 |
| | | | SVKG | | GLDV | |
| H2/L2-36 | GFTFSSYGMH | 1251 | VISYKGSNKYYAD | 1178 | GGSGYALHDDYY | 1119 |
| | | | SVKG | | GLDV | |
| H2/L2-34 | GFTFSSYGMH | 1251 | VISYTGTKKYYAD | 1179 | GGSGYALHDDYY | 1119 |
| | | | SVKG | | GLDV | |
| H2/L2-68 | GFTFSSYGMH | 1251 | VISYRGFNKYYAD | 1180 | GGSGYALHDDYY | 1241 |
| | | | SVKG | | GQDV | |
| H2/L2-18 | GFTFSSYGMH | 1251 | VISYKGSHKYYAD | 1181 | GGSGYALHDDYY | 1119 |
| | | | SVKG | | GLDV | |
| H2/L2-47 | GFTFSSYGMH | 1251 | VISYKGSNKYYAD | 1178 | GGSGYALHDDYY | 1119 |
| | | | SVKG | | GLDV | |
| H2/L2-20 | GFTVSSYGMH | 1250 | VISYTGSNKYYAD | 1175 | GGSGYALHDDYY | 1119 |
| | | | SVKG | | GLDV | |
| H2/L2-80 | GFTFSSYGMH | 1251 | VISYTGSNKYYAD | 1175 | GGSGYALHDDYY | 1119 |
| | | | SVKG | | GLDV | |
| H2/L2-83 | GFTFSSYGMH | 1251 | VISYKGSNKYYAD | 1178 | GGSGYALHDDYY | 1119 |
| | | | SVKG | | GLDV | |
| H3-1 | GFTFSSYGMH | 1251 | VISYDDAHKYYAD | 1182 | GGSGYALHDQYKP | 1242 |
| | | | SVKG | | VDV | |
| H3-2 | GFTFSSYGMH | 1251 | VISYNDLNKYYAD | 1183 | GGSGYALHDFQDP | 1243 |
| | | | SVKG | | TDV | |
| H3-3 | GFTVSSYGMH | 1250 | VISYSGSNKYYAD | 1184 | GGSGYALHDQYKP | 1242 |
| | | | SVKG | | VDV | |
| H3-4 | GFTFSSYGMH | 1251 | VISYDDAHKYYAD | 1182 | GGSGYALHDQYKP | 1242 |
| | | | SVKG | | VDV | |
| H3-5 | GFTFSSYGMH | 1251 | VISYTGANKYYAD | 1185 | GGSGYNLHDDYY | 1244 |
| | | | SVKG | | GLDV | |
| H3-6 | GFTFSSYGMH | 1251 | VISYDDAHKYYAD | 1182 | GGSGYALHDQYKP | 1242 |
| | | | SVKG | | VDV | |
| H3-7 | GFTLSSYGMH | 1252 | VISYTGSNKYYAD | 1175 | GGSGYEFHEDYY | 1245 |
| | | | SVKG | | GLDV | |
| H3-8 | GFTFSSYGMH | 1251 | VISYDDAHKYYAD | 1182 | GGSGYALHDQYKP | 1242 |
| | | | SVKG | | VDV | |
| H3-9 | GFTFSSYGMH | 1251 | VISYDDAHKYYAD | 1182 | GGSGYALHDQYKP | 1242 |
| | | | SVKG | | VDV | |
| H3-10 | GFTFSSYGMH | 1251 | VISYNDLNKYYAD | 1183 | GGSGYEFQGDYY | 1246 |
| | | | SVKG | | GLDV | |
| H3-11 | GFTFSSYGMH | 1251 | VISYNDANKYYAD | 1186 | GGSGYELRDDYY | 1247 |
| | | | SVKG | | GLDV | |
| H3-12 | GFTFSSYGMH | 1251 | VISYDESNKYYAD | 1187 | GGSGYEVDQDYY | 1248 |
| | | | SVKG | | GLDV | |
| H3-13 | GFTFSSYGMH | 1251 | VISYDDAHKYYAD | 1182 | GGSGYALHDQYKP | 1242 |
| | | | SVKG | | VDV | |
| H3-14 | GFTFSSYGMH | 1251 | VISYDDAHKYYAD | 1182 | GGSGYALHDQYKP | 1242 |
| | | | SVKG | | VDV | |
| H3-15 | GFTVSSYGMH | 1250 | VISYDDANKYYAD | 1188 | GGSGYAYDGDYY | 1249 |
| | | | SVKG | | GLDV | |

TABLE 15N-1(a)
AB3 family BCMA Binders-CDR-L1 and CDR-L3 sequences according to combination
of Chothia and IMGT numbering schemes and CDR-L2 expanded sequences

| | | SEQ ID | | SEQ ID | | SEQ ID |
|---|---|---|---|---|---|---|
| Binder | CDR-L1 | NO: | CDR-L2: | NO: | CDR-L3 | NO: |
| AB3 | TSSDVGGYNY | 1095 | DVSNRLRGVS | 1216 | SSYTSSSALYV | 1173 |
| PI-61 | TSSDVGGYNY | 1095 | DVSNRPSGVS | 1217 | SSYTSSSTLYV | 1174 |
| H2/L2-22 | TSSDVGGYNY | 1095 | EVSNRLSGVS | 1218 | SSYTSSSTLYV | 1174 |
| H2/L2-88 | TSSDVGGYNY | 1095 | EVSNRLRGVS | 1219 | SSYTSSSALYV | 1173 |
| H2/L2-36 | TSSDVGGYNY | 1095 | EVSNRLRGVS | 1219 | SSYTSSSTLYV | 1174 |
| H2/L2-34 | TSSDVGGYNY | 1095 | DVSNRPWGVS | 1220 | SSYTSSSALYV | 1173 |
| H2/L2-68 | TSSDVGGYNY | 1095 | DVSNRLSGVS | 1221 | SSYTSSSTLYV | 1174 |
| H2/L2-18 | TSSDVGGYNY | 1095 | DVSNRPWGVS | 1220 | SSYTSSSTLYV | 1174 |
| H2/L2-47 | TSSDVGGYNY | 1095 | DVSNRPWGVS | 1220 | SSYTSSSTLYV | 1174 |
| H2/L2-20 | TSSDVGGYNY | 1095 | DVSNRLRGVS | 1216 | SSYTSSSALYV | 1173 |
| H2/L2-80 | TSSDVGGYNY | 1095 | DVSNRAWGVS | 1222 | SSYTSSSALYV | 1173 |
| H2/L2-83 | TSSDVGGYNY | 1095 | EVSNRLRGVS | 1219 | SSYTSSSTLYV | 1174 |
| H3-1 | TSSDVGGYNY | 1095 | EVSNRLRGVS | 1219 | SSYTSSSTLYV | 1174 |
| H3-2 | TSSDVGGYNY | 1095 | EVSNRLRGVS | 1219 | SSYTSSSTLYV | 1174 |
| H3-3 | TSSDVGGYNY | 1095 | EVSNRLRGVS | 1219 | SSYTSSSTLYV | 1174 |
| H3-4 | TSSDVGGYNY | 1095 | EVSNRLRGVS | 1219 | SSYTSSSALYV | 1173 |
| H3-5 | TSSDVGGYNY | 1095 | EVSNRLSGVS | 1218 | SSYTSSSTLYV | 1174 |
| H3-6 | TSSDVGGYNY | 1095 | EVSNRLRGVS | 1219 | SSYTSSSALYV | 1173 |
| H3-7 | TSSDVGGYNY | 1095 | EVSNRLRGVS | 1219 | SSYTSSSTLYV | 1174 |
| H3-8 | TSSDVGGYNY | 1095 | EVSNRLRGVS | 1219 | SSYTSSSTLYV | 1174 |
| H3-9 | TSSDVGGYNY | 1095 | EVSNRLRGVS | 1219 | SSYTSSSTLYV | 1174 |
| H3-10 | TSSDVGGYNY | 1095 | EVSNRLRGVS | 1219 | SSYTSSSTLYV | 1174 |
| H3-11 | TSSDVGGYNY | 1095 | EVSNRLRGVS | 1219 | SSYTSSSTLYV | 1174 |
| H3-12 | TSSDVGGYNY | 1095 | EVSNRLRGVS | 1219 | SSYTSSSTLYV | 1174 |
| H3-13 | TSSDVGGYNY | 1095 | EVSNRLRGVS | 1219 | SSYTSSSTLYV | 1174 |
| H3-14 | TSSDVGGYNY | 1095 | EVSNRLSGVS | 1218 | SSYTSSSALYV | 1173 |
| H3-15 | TSSDVGGYNY | 1095 | EVSNRLGGVS | 1223 | SSYTSSSALYV | 1173 |

TABLE 15N-1(b)
AB3 family BCMA Binders-Light Chain CDR sequences according to
combination of Chothia and IMGT numbering schemes

| | | SEQ ID | | SEQ ID | | SEQ ID |
|---|---|---|---|---|---|---|
| Binder | CDR-L1 | NO: | CDR-L2: | NO: | CDR-L3 | NO: |
| AB3 | TSSDVGGYNY | 1095 | DVS | 1099 | SSYTSSSALYV | 1173 |
| PI-61 | TSSDVGGYNY | 1095 | DVS | 1099 | SSYTSSSTLYV | 1174 |
| H2/L2-22 | TSSDVGGYNY | 1095 | EVS | 580 | SSYTSSSTLYV | 1174 |
| H2/L2-88 | TSSDVGGYNY | 1095 | EVS | 580 | SSYTSSSALYV | 1173 |
| H2/L2-36 | TSSDVGGYNY | 1095 | EVS | 580 | SSYTSSSTLYV | 1174 |
| H2/L2-34 | TSSDVGGYNY | 1095 | DVS | 1099 | SSYTSSSALYV | 1173 |
| H2/L2-68 | TSSDVGGYNY | 1095 | DVS | 1099 | SSYTSSSTLYV | 1174 |
| H2/L2-18 | TSSDVGGYNY | 1095 | DVS | 1099 | SSYTSSSTLYV | 1174 |
| H2/L2-47 | TSSDVGGYNY | 1095 | DVS | 1099 | SSYTSSSTLYV | 1174 |
| H2/L2-20 | TSSDVGGYNY | 1095 | DVS | 1099 | SSYTSSSALYV | 1173 |
| H2/L2-80 | TSSDVGGYNY | 1095 | DVS | 1099 | SSYTSSSALYV | 1173 |
| H2/L2-83 | TSSDVGGYNY | 1095 | EVS | 580 | SSYTSSSTLYV | 1174 |
| H3-1 | TSSDVGGYNY | 1095 | EVS | 580 | SSYTSSSTLYV | 1174 |
| H3-2 | TSSDVGGYNY | 1095 | EVS | 580 | SSYTSSSTLYV | 1174 |
| H3-3 | TSSDVGGYNY | 1095 | EVS | 580 | SSYTSSSTLYV | 1174 |
| H3-4 | TSSDVGGYNY | 1095 | EVS | 580 | SSYTSSSALYV | 1173 |
| H3-5 | TSSDVGGYNY | 1095 | EVS | 580 | SSYTSSSTLYV | 1174 |
| H3-6 | TSSDVGGYNY | 1095 | EVS | 580 | SSYTSSSALYV | 1173 |
| H3-7 | TSSDVGGYNY | 1095 | EVS | 580 | SSYTSSSTLYV | 1174 |
| H3-8 | TSSDVGGYNY | 1095 | EVS | 580 | SSYTSSSTLYV | 1174 |
| H3-9 | TSSDVGGYNY | 1095 | EVS | 580 | SSYTSSSTLYV | 1174 |
| H3-10 | TSSDVGGYNY | 1095 | EVS | 580 | SSYTSSSTLYV | 1174 |
| H3-11 | TSSDVGGYNY | 1095 | EVS | 580 | SSYTSSSTLYV | 1174 |
| H3-12 | TSSDVGGYNY | 1095 | EVS | 580 | SSYTSSSTLYV | 1174 |
| H3-13 | TSSDVGGYNY | 1095 | EVS | 580 | SSYTSSSTLYV | 1174 |
| H3-14 | TSSDVGGYNY | 1095 | EVS | 580 | SSYTSSSALYV | 1173 |
| H3-15 | TSSDVGGYNY | 1095 | EVS | 580 | SSYTSSSALYV | 1173 |

TABLE 15N-2
AB3 family BCMA Binders-Heavy Chain CDR sequences according to combination
of Chothia and IMGT numbering schemes

| | | SEQ ID | | SEQ ID | | SEQ ID |
|---|---|---|---|---|---|---|
| Binder | CDR-H1 | NO: | CDR-H2: | NO: | CDR-H3 | NO: |
| AB3 | GFTVSSYG | 1224 | ISYTGSNK | 1227 | GGSGYALHDDYYG | 1119 |
| | | | | | LDV | |
| PI-61 | GFTFSSYG | 1225 | ISYDGSNK | 1228 | GGSGYALHDDYYG | 1119 |
| | | | | | LDV | |
| H2/L2-22 | GFTFSSYG | 1225 | ISYHGSNK | 1229 | GGSGYALHDDYYG | 1119 |
| | | | | | LDV | |
| H2/L2-88 | GFTFSSYG | 1225 | ISYKGSNK | 1230 | GGSGYALHDDYYG | 1119 |
| | | | | | LDV | |
| H2/L2-36 | GFTFSSYG | 1225 | ISYKGSNK | 1230 | GGSGYALHDDYYG | 1119 |
| | | | | | LDV | |
| H2/L2-34 | GFTFSSYG | 1225 | ISYTGTKK | 1231 | GGSGYALHDDYYG | 1119 |
| | | | | | LDV | |
| H2/L2-68 | GFTFSSYG | 1225 | ISYRGFNK | 1232 | GGSGYALHDDYYG | 1241 |
| | | | | | QDV | |
| H2/L2-18 | GFTFSSYG | 1225 | ISYKGSHK | 1233 | GGSGYALHDDYYG | 1119 |
| | | | | | LDV | |
| H2/L2-47 | GFTFSSYG | 1225 | ISYKGSNK | 1230 | GGSGYALHDDYYG | 1119 |
| | | | | | LDV | |
| H2/L2-20 | GFTVSSYG | 1224 | ISYTGSNK | 1227 | GGSGYALHDDYYG | 1119 |
| | | | | | LDV | |
| H2/L2-80 | GFTFSSYG | 1225 | ISYTGSNK | 1227 | GGSGYALHDDYYG | 1119 |
| | | | | | LDV | |
| H2/L2-83 | GFTFSSYG | 1225 | ISYKGSNK | 1230 | GGSGYALHDDYYG | 1119 |
| | | | | | LDV | |
| H3-1 | GFTFSSYG | 1225 | ISYDDAHK | 1234 | GGSGYALHDQYKP | 1242 |
| | | | | | VDV | |
| H3-2 | GFTFSSYG | 1225 | ISYNDLNK | 1235 | GGSGYALHDFQDP | 1243 |
| | | | | | TDV | |
| H3-3 | GFTVSSYG | 1224 | ISYSGSNK | 1236 | GGSGYALHDQYKP | 1242 |
| | | | | | VDV | |
| H3-4 | GFTFSSYG | 1225 | ISYDDAHK | 1234 | GGSGYALHDQYKP | 1242 |
| | | | | | VDV | |
| H3-5 | GFTFSSYG | 1225 | ISYTGANK | 1237 | GGSGYNLHDDYYG | 1244 |
| | | | | | LDV | |
| H3-6 | GFTFSSYG | 1225 | ISYDDAHK | 1234 | GGSGYALHDQYKP | 1242 |
| | | | | | VDV | |
| H3-7 | GFTLSSYG | 1226 | ISYTGSNK | 1227 | GGSGYEFHEDYYG | 1245 |
| | | | | | LDV | |
| H3-8 | GFTFSSYG | 1225 | ISYDDAHK | 1234 | GGSGYALHDQYKP | 1242 |
| | | | | | VDV | |
| H3-9 | GFTFSSYG | 1225 | ISYDDAHK | 1234 | GGSGYALHDQYKP | 1242 |
| | | | | | VDV | |
| H3-10 | GFTFSSYG | 1225 | ISYNDLNK | 1235 | GGSGYEFQGDYY | 1246 |
| | | | | | GLDV | |
| H3-11 | GFTFSSYG | 1225 | ISYNDANK | 1238 | GGSGYELRDDYYG | 1247 |
| | | | | | LDV | |
| H3-12 | GFTFSSYG | 1225 | ISYDESNK | 1239 | GGSGYEVDQDYY | 1248 |
| | | | | | GLDV | |
| H3-13 | GFTFSSYG | 1225 | ISYDDAHK | 1234 | GGSGYALHDQYKP | 1242 |
| | | | | | VDV | |
| H3-14 | GFTFSSYG | 1225 | ISYDDAHK | 1234 | GGSGYALHDQYKP | 1242 |
| | | | | | VDV | |
| H3-15 | GFTVSSYG | 1224 | ISYDDANK | 1240 | GGSGYAYDGDYY | 1249 |
| | | | | | GLDV | |

TABLE 15O-1
BCMA Binders - Light chain variable sequences

| | | SEQ |
|---|---|---|
| | | ID |
| Binder | Sequence | NO: |
| AB1 | DIQMTQSPSSLSASVGDRVTITCRASQSISSYLNWYQQKPGKAPKLLIYAASSL | 1253 |
| | QSGVPSRFSGSGSGTDFTLTISSLQPEDFATYYCQQSYSSPLTFGQGTKVEIK | |
| AB2 | DIQMTQSPSSLSASVGDRVTITCRASQSISSYLNWYQQKPGKAPKLLIYAASSL | 1254 |
| | QSGVPSRFSGSGSGTDFTLTISSLQPEDFATYYCQQSYSTPLTFGQGTKVEIK | |
| R1F2 | DIQMTQSPSSLSASVGDRVTITCRASQSISSYLNWYQQKPGKAPKLLIYAASSL | 1254 |
| | QSGVPSRFSGSGSGTDFTLTISSLQPEDFATYYCQQSYSTPLTFGQGTKVEIK | |
| PALF03 | DIQMTQSPSSLSASVGDRVTITCRASQSISSYLNWYQQKPGKAPKLLIYAASSL | 1255 |
| | QSGVPSRFSGSGSGTDFTLTISSLQPEDFATYYCQQSYGSPPTFGQGTKVEIK | |
| PALF04 | DIQMTQSPSSLSASVGDRVTITCRASQSISSYLNWYQQKPGKAPKLLIYAASSL | 1256 |
| | QSGVPSRFSGSGSGTDFTLTISSLQPEDFATYYCQQSYDSPLTFGQGTKVEIK | |
| PALF05 | DIQMTQSPSSLSASVGDRVTITCRASQSISSYLNWYQQKPGKAPKLLIYAASSL | 1257 |
| | QSGVPSRFSGSGSGTDFTLTISSLQPEDFATYYCQQSYYSPLTFGQGTKVEIK | |
| PALF06 | DIQMTQSPSSLSASVGDRVTITCRASQSISSYLNWYQQKPGKAPKLLIYAASSL | 1258 |
| | QSGVPSRFSGSGSGTDFTLTISSLQPEDFATYYCQQSYYAPLTFGQGTKVEIK | |
| PALF07 | DIQMTQSPSSLSASVGDRVTITCRASQSISSYLNWYQQKPGKAPKLLIYAASSL | 1259 |
| | QSGVPSRFSGSGSGTDFTLTISSLQPEDFATYYCQQSYASPLTFGQGTKVEIK | |
| PALF08 | DIQMTQSPSSLSASVGDRVTITCRASQSISSYLNWYQQKPGKAPKLLIYAASSL | 1260 |
| | QSGVPSRFSGSGSGTDFTLTISSLQPEDFATYYCQQSYGSPLTFGQGTKVEIK | |
| PALF09 | DIQMTQSPSSLSASVGDRVTITCRASQSISSYLNWYQQKPGKAPKLLIYAASSL | 1261 |
| | QSGVPSRFSGSGSGTDFTLTISSLQPEDFATYYCQQSYDAPLTFGQGTKVEIK | |
| PALF12 | DIQMTQSPSSLSASVGDRVTITCRASQSISSYLNWYQQKPGKAPKLLIYAASSL | 1254 |
| | QSGVPSRFSGSGSGTDFTLTISSLQPEDFATYYCQQSYSTPLTFGQGTKVEIK | |
| PALF13 | DIQMTQSPSSLSASVGDRVTITCRASQSISSYLNWYQQKPGKAPKLLIYAASSL | 1254 |
| | QSGVPSRFSGSGSGTDFTLTISSLQPEDFATYYCQQSYSTPLTFGQGTKVEIK | |
| PALF14 | DIQMTQSPSSLSASVGDRVTITCRASQSISSYLNWYQQKPGKAPKLLIYAASSL | 1254 |
| | QSGVPSRFSGSGSGTDFTLTISSLQPEDFATYYCQQSYSTPLTFGQGTKVEIK | |
| PALF15 | DIQMTQSPSSLSASVGDRVTITCRASQSISSYLNWYQQKPGKAPKLLIYAASSL | 1254 |
| | QSGVPSRFSGSGSGTDFTLTISSLQPEDFATYYCQQSYSTPLTFGQGTKVEIK | |
| PALF16 | DIQMTQSPSSLSASVGDRVTITCRASQSISSYLNWYQQKPGKAPKLLIYAASSL | 1254 |
| | QSGVPSRFSGSGSGTDFTLTISSLQPEDFATYYCQQSYSTPLTFGQGTKVEIK | |
| PALF17 | DIQMTQSPSSLSASVGDRVTITCRASQSISSYLNWYQQKPGKAPKLLIYAASSL | 1254 |
| | QSGVPSRFSGSGSGTDFTLTISSLQPEDFATYYCQQSYSTPLTFGQGTKVEIK | |
| PALF18 | DIQMTQSPSSLSASVGDRVTITCRASQSISSYLNWYQQKPGKAPKLLIYAASSL | 1254 |
| | QSGVPSRFSGSGSGTDFTLTISSLQPEDFATYYCQQSYSTPLTFGQGTKVEIK | |
| PALF19 | DIQMTQSPSSLSASVGDRVTITCRASQSISSYLNWYQQKPGKAPKLLIYAASSL | 1254 |
| | QSGVPSRFSGSGSGTDFTLTISSLQPEDFATYYCQQSYSTPLTFGQGTKVEIK | |
| PALF20 | DIQMTQSPSSLSASVGDRVTITCRASQSISSYLNWYQQKPGKAPKLLIYAASSL | 1254 |
| | QSGVPSRFSGSGSGTDFTLTISSLQPEDFATYYCQQSYSTPLTFGQGTKVEIK | |
| AB3 | QSALTQPASVSGSPGQSITISCTGTSSDVGGYNYVSWYQQHPGKAPKLMIYDVS | 1262 |
| | NRLRGVSNRFSGSKSGNTASLTISGLQAEDEADYYCSSYTSSSALYVFGSGTKV | |
| | TVL | |
| PI-61 | QSALTQPASVSGSPGQSITISCTGTSSDVGGYNYVSWYQQHPGKAPKLMIYDVS | 1263 |
| | NRPSGVSNRFSGSKSGNTASLTISGLQAEDEADYYCSSYTSSSTLYVFGSGTKV | |
| | TVL | |
| H2/L2-22 | QSALTQPASVSGSPGQSITISCTGTSSDVGGYNYVSWYQQHPGKAPKLMIYEVS | 1264 |
| | NRLSGVSNRFSGSKFGNTASLTISGLQAEDEADYYCSSYTSSSTLYVFGSGTKV | |
| | TVL | |
| H2/L2-88 | QSALTQPASVSGSPGQSITISCTGTSSDVGGYNYVSWYQQHPGKAPKLMIYEVS | 1265 |
| | NRLRGVSNRFSGSKFGNTASLTISGLQAEDEADYYCSSYTSSSALYVFGSGTKV | |
| | TVL | |
| H2/L2-36 | QSALTQPASVSGSPGQSITISCTGTSSDVGGYNYVSWYQQHPGKAPKLMIYEVS | 1266 |
| | NRLRGVSNRFSGSKFGNTASLTISGLQAEDEADYYCSSYTSSSTLYVFGSGTKV | |
| | TVL | |
| H2/L2-34 | QSALTQPASVSGSPGQSITISCTGTSSDVGGYNYVSWYQQHPGKAPKLMIYDVS | 1267 |
| | NRPWGVSNRFSGSKFGNTASLTISGLQAEDEADYYCSSYTSSSALYVFGSGTKV | |
| | TVM | |
| H2/L2-68 | QSALTQPASVSGSPGQSITISCTGTSSDVGGYNYVSWYQQHPGKAPKLMIYDVS | 1268 |
| | NRLSGVSNRFSGSKFGNTASLTISGLQAEDEADYYCSSYTSSSTLYVFGSGTKV | |
| | TVL | |
| H2/L2-18 | QSALTQPASVSGSPGQSITISCTGTSSDVGGYNYVSWYQQHPGKAPKLMIYDVS | 1269 |
| | NRPWGVSNRFSGSKFGNTASLTISGLQAEDEADYYCSSYTSSSTLYVFGSGTKV | |
| | TVL | |
| H2/L2-47 | QSALTQPASVSGSPGQSITISCTGTSSDVGGYNYVSWYQQHPGKAPKLMIYDVS | 1269 |
| | NRPWGVSNRFSGSKFGNTASLTISGLQAEDEADYYCSSYTSSSTLYVFGSGTKV | |
| | TVL | |
| H2/L2-20 | QSALTQPASVSGSPGQSITISCTGTSSDVGGYNYVSWYQQHPGKAPKLMIYDVS | 1270 |
| | NRLRGVSNRFSGSKFGNTASLTISGLQAEDEADYYCSSYTSSSALYVFGSGTKV | |
| | TVL | |
| H2/L2-80 | QSALTQPASVSGSPGQSITISCTGTSSDVGGYNYVSWYQQHPGKAPKLMIYDVS | 1271 |
| | NRAWGVSNRFSGSKFGNTASLTISGLQAEDEADYYCSSYTSSSALYVFGSGTKV | |
| | TVL | |
| H2/L2-83 | QSALTQPASVSGSPGQSITISCTGTSSDVGGYNYVSWYQQHPGKAPKLMIYEVS | 1266 |
| | NRLRGVSNRFSGSKFGNTASLTISGLQAEDEADYYCSSYTSSSTLYVFGSGTKV | |
| | TVL | |
| H3-1 | QSALTQPASVSGSPGQSITISCTGTSSDVGGYNYVSWYQQHPGKAPKLMIYEVS | 1266 |
| | NRLRGVSNRFSGSKFGNTASLTISGLQAEDEADYYCSSYTSSSTLYVFGSGTKV | |
| | TVL | |
| H3-2 | QSALTQPASVSGSPGQSITISCTGTSSDVGGYNYVSWYQQHPGKAPKLMIYEVS | 1266 |
| | NRLRGVSNRFSGSKFGNTASLTISGLQAEDEADYYCSSYTSSSTLYVFGSGTKV | |
| | TVL | |
| H3-3 | QSALTQPASVSGSPGQSITISCTGTSSDVGGYNYVSWYQQHPGKAPKLMIYEVS | 1266 |
| | NRLRGVSNRFSGSKFGNTASLTISGLQAEDEADYYCSSYTSSSTLYVFGSGTKV | |
| | TVL | |
| H3-4 | QSALTQPASVSGSPGQSITISCTGTSSDVGGYNYVSWYQQHPGKAPKLMIYEVS | 1265 |
| | NRLRGVSNRFSGSKFGNTASLTISGLQAEDEADYYCSSYTSSSALYVFGSGTKV | |
| | TVL | |
| H3-5 | QSALTQPASVSGSPGQSITISCTGTSSDVGGYNYVSWYQQHPGKAPKLMIYEVS | 1264 |
| | NRLSGVSNRFSGSKFGNTASLTISGLQAEDEADYYCSSYTSSSTLYVFGSGTKV | |
| | TVL | |
| H3-6 | QSALTQPASVSGSPGQSITISCTGTSSDVGGYNYVSWYQQHPGKAPKLMIYEVS | 1265 |
| | NRLRGVSNRFSGSKFGNTASLTISGLQAEDEADYYCSSYTSSSALYVFGSGTKV | |
| | TVL | |
| H3-7 | QSALTQPASVSGSPGQSITISCTGTSSDVGGYNYVSWYQQHPGKAPKLMIYEVS | 1266 |
| | NRLRGVSNRFSGSKFGNTASLTISGLQAEDEADYYCSSYTSSSTLYVFGSGTKV | |
| | TVL | |
| H3-8 | QSALTQPASVSGSPGQSITISCTGTSSDVGGYNYVSWYQQHPGKAPKLMIYEVS | 1266 |
| | NRLRGVSNRFSGSKFGNTASLTISGLQAEDEADYYCSSYTSSSTLYVFGSGTKV | |
| | TVL | |
| H3-9 | QSALTQPASVSGSPGQSITISCTGTSSDVGGYNYVSWYQQHPGKAPKLMIYEVS | 1272 |
| | NRLRGVSNRFSGSKFGNTASLTISGLQAEDEAYYYCSSYTSSSTLYVFGSGTKV | |
| | TVL | |
| H3-10 | QSALTQPASVSGSPGQSITISCTGTSSDVGGYNYVSWYQQHPGKAPKLMIYEVS | 1266 |
| | NRLRGVSNRFSGSKFGNTASLTISGLQAEDEADYYCSSYTSSSTLYVFGSGTKV | |
| | TVL | |
| H3-11 | QSALTQPASVSGSPGQSITISCTGTSSDVGGYNYVSWYQQHPGKAPKLMIYEVS | 1266 |
| | NRLRGVSNRFSGSKFGNTASLTISGLQAEDEADYYCSSYTSSSTLYVFGSGTKV | |
| | TVL | |
| H3-12 | QSALTQPASVSGSPGQSITISCTGTSSDVGGYNYVSWYQQHPGKAPKLMIYEVS | 1266 |
| | NRLRGVSNRFSGSKFGNTASLTISGLQAEDEADYYCSSYTSSSTLYVFGSGTKV | |
| | TVL | |
| H3-13 | QSALTQPASVSGSPGQSITISCTGTSSDVGGYNYVSWYQQHPGKAPKLMIYEVS | 1266 |
| | NRLRGVSNRFSGSKFGNTASLTISGLQAEDEADYYCSSYTSSSTLYVFGSGTKV | |
| | TVL | |
| H3-14 | QSALTQPASVSGSPGQSITISCTGTSSDVGGYNYVSWYQQHPGKAPKLMIYEVS | 1273 |
| | NRLSGVSNRFSGSKFGNTASLTISGLQAEDEADYYCSSYTSSSALYVFGSGTKV | |
| | TVL | |
| H3-15 | QSALTQPASVSGSPGQSITISCTGTSSDVGGYNYVSWYQQHPGKAPKLMIYEVS | 1274 |
| | NRLGGVSNRFSGSKFGNTASLTISGLQAEDEADYYCSSYTSSSALYVFGSGTKV | |
| | TVL | |

TABLE 15O-2
BCMA Binders - Heavy chain variable sequences

| | | SEQ |
|---|---|---|
| | | ID |
| Binder | Sequence | NO: |
| AB1 | EVQLLESGGGLVQPGGSLRLSCAASGFTFSSYAMSWVRQAPGKGLEWVSAISGS | 1275 |
| | GGSTYYADSVKGRFTISRDNSKNTLYLQMNSLRAEDTAVYYCARREWWYDDWYL | |
| | DYWGQGTLVTVSS | |
| AB2 | EVQLLESGGGLVQPGGSLRLSCAASGFTFSSYAMSWVRQAPGKGLEWVSAISES | 1276 |
| | GGRAAYADSVKGRFTISRDNSKNTLYLQMNSLRAEDTAVYYCARREWWYDDWYL | |
| | DYWGQGTLVTVSS | |
| R1F2 | EVQLLESGGGLVQPGGSLRLSCAASGFTFSSYAMSWVRQAPGKGLEWVSAISGS | 1275 |
| | GGSTYYADSVKGRFTISRDNSKNTLYLQMNSLRAEDTAVYYCARREWWYDDWYL | |
| | DYWGQGTLVTVSS | |
| PALF03 | EVQLLESGGGLVQPGGSLRLSCAASGFTFSSYAMSWVRQAPGKGLEWVSAISGS | 1275 |
| | GGSTYYADSVKGRFTISRDNSKNTLYLQMNSLRAEDTAVYYCARREWWYDDWYL | |
| | DYWGQGTLVTVSS | |
| PALF04 | EVQLLESGGGLVQPGGSLRLSCAASGFTFSSYAMSWVRQAPGKGLEWVSAISGS | 1275 |
| | GGSTYYADSVKGRFTISRDNSKNTLYLQMNSLRAEDTAVYYCARREWWYDDWYL | |
| | DYWGQGTLVTVSS | |
| PALF05 | EVQLLESGGGLVQPGGSLRLSCAASGFTFSSYAMSWVRQAPGKGLEWVSAISGS | 1275 |
| | GGSTYYADSVKGRFTISRDNSKNTLYLQMNSLRAEDTAVYYCARREWWYDDWYL | |
| | DYWGQGTLVTVSS | |
| PALF06 | EVQLLESGGGLVQPGGSLRLSCAASGFTFSSYAMSWVRQAPGKGLEWVSAISGS | 1275 |
| | GGSTYYADSVKGRFTISRDNSKNTLYLQMNSLRAEDTAVYYCARREWWYDDWYL | |
| | DYWGQGTLVTVSS | |
| PALF07 | EVQLLESGGGLVQPGGSLRLSCAASGFTFSSYAMSWVRQAPGKGLEWVSAISGS | 1275 |
| | GGSTYYADSVKGRFTISRDNSKNTLYLQMNSLRAEDTAVYYCARREWWYDDWYL | |
| | DYWGQGTLVTVSS | |
| PALF08 | EVQLLESGGGLVQPGGSLRLSCAASGFTFSSYAMSWVRQAPGKGLEWVSAISGS | 1275 |
| | GGSTYYADSVKGRFTISRDNSKNTLYLQMNSLRAEDTAVYYCARREWWYDDWYL | |
| | DYWGQGTLVTVSS | |
| PALF09 | EVQLLESGGGLVQPGGSLRLSCAASGFTFSSYAMSWVRQAPGKGLEWVSAISGS | 1275 |
| | GGSTYYADSVKGRFTISRDNSKNTLYLQMNSLRAEDTAVYYCARREWWYDDWYL | |
| | DYWGQGTLVTVSS | |
| PALF12 | EVQLLESGGGLVQPGGSLRLSCAASGFTFSSYAMSWVRQAPGKGLEWVSAISGS | 1277 |
| | GGRAAYADSVKGRFTISRDNSKNTLYLQMNSLRAEDTAVYYCARREWWYDDWYL | |
| | DYWGQGTLVTVSS | |
| PALF13 | EVQLLESGGGLVQPGGSLRLSCAASGFTFSSYAMSWVRQAPGKGLEWVSAISES | 1278 |
| | GDVEAYADSVKGRFTISRDNSKNTLYLQMNSLRAEDTAVYYCARREWWYDDWYL | |
| | DYWGQGTLVTVSS | |
| PALF14 | EVQLLESGGGLVQPGGSLRLSCAASGFTFSSYAMSWVRQAPGKGLEWVSAISEA | 1279 |
| | GETTSYADSVKGRFTISRDNSKNTLYLQMNSLRAEDTAVYYCARREWWYDDWYL | |
| | DYWGQGTLVTVSS | |
| PALF15 | EVQLLESGGGLVQPGGSLRLSCAASGFTFSSYAMSWVRQAPGKGLEWVSAISEH | 1280 |
| | GHYTSYADSVKGRFTISRDNSKNTLYLQMNSLRAEDTAVYYCARREWWYDDWYL | |
| | DYWGQGTLVTVSS | |
| PALF16 | EVQLLESGGGLVQPGGSLRLSCAASGFTFSSYAMSWVRQAPGKGLEWVSAISGS | 1281 |
| | GHTAAYADSVKGRFTISRDNSKNTLYLQMNSLRAEDTAVYYCARREWWYDDWYL | |
| | DYWGQGTLVTVSS | |
| PALF17 | EVQLLESGGGLVQPGGSLRLSCAASGFTFSSYAMSWVRQAPGKGLEWVSAISGS | 1282 |
| | GRTHAYADSVKGRFTISRDNSKNTLYLQMNSLRAEDTAVYYCARREWWYDDWYL | |
| | DYWGQGTLVTVSS | |
| PALF18 | EVQLLESGGGLVQPGGSLRLSCAASGFTFSSYAMSWVRQAPGKGLEWVSAISAE | 1283 |
| | GGVRAYADSVKGRFTISRDNSKNTLYLQMNSLRAEDTAVYYCARREWWYDDWYL | |
| | DYWGQGTLVTVSS | |
| PALF19 | EVQLLESGGGLVQPGGSLRLSCAASGFTFSSYAMSWVRQAPGKGLEWVSAISGS | 1284 |
| | GGTTAYADSVKGRFTISRDNSKNTLYLQMNSLRAEDTAVYYCARREWWYDDWYL | |
| | DYWGQGTLVTVSS | |
| PALF20 | EVQLLESGGGLVQPGGSLRLSCAASGFTFSSYAMSWVRQAPGKGLEWVSAISGS | 1285 |
| | GATTAYADSVKGRFTISRDNSKNTLYLQMNSLRAEDTAVYYCARREWWYDDWYL | |
| | DYWGQGTLVTVSS | |
| AB3 | QVQLVESGGGVVQPGRSLRLSCAASGFTVSSYGMHWVRQAPGKGLEWVAVISYT | 1286 |
| | GSNKYYADSVKGRFTISRDNSKNTLYLQMNSLRAEDTAVYYCGGSGYALHDDYY | |
| | GLDVWGQGTLVTVSS | |
| PI-61 | QVQLQESGGGVVQPGRSLRLSCAASGFTFSSYGMHWVRQAPGKGLEWVAVISYD | 1287 |
| | GSNKYYADSVKGRFTISRDNSKNTLYLQMNSLRAEDTAVYYCGGSGYALHDDYY | |
| | GLDVWGQGTLVTVSS | |
| H2/L2-22 | QAQLQSSEGGVVQPGRSLRLSCAASGFTFSSYGMHWVRQAPGKGLEWVAVISYH | 1288 |
| | GSNKYYADSVKGRFTISRDNSKNTLYLQMNSLRAEDTAVYYCGGSGYALHDDYY | |
| | GLDVWGQGTLVTVSSS | |
| H2/L2-88 | QVQLQSSEGGVVQPGRSLRLSCAASGFTFSSYGMHWVRQAPGKGLEWVAVISYK | 1289 |
| | GSNKYYADSVKGRFTISRDNSKNTLYLQMNSLRAEDTAVYYCGGSGYALHDDYY | |
| | GLDVWGQGTLVTVSS | |
| H2/L2-36 | QAQLQSSGGGVVQPGRSLRLSCAASGFTFSSYGMHWVRQAPGKGLEWVAVISYK | 1290 |
| | GSNKYYADSVKGRFTISRDNSKNTLYLQMNSLRAEDTAVYYCGGSGYALHDDYY | |
| | GLDVWGQGTLVTVSS | |
| H2/L2-34 | QVQLQDSEGGVVQPGRSLRLSCAASGFTFSSYGMHWVRQAPGKGLEWVAVISYT | 1291 |
| | GTKKYYADSVKGRFTISRDNSKNTLYLQMNSLRAEDTAVYYCGGSGYALHDDYY | |
| | GLDVWGQGTLVTVSS | |
| H2/L2-68 | QAQLQSSEGGVVQPGRSLRLSCAASGFTFSSYGMHWVRQAPGKGLEWVAVISYR | 1292 |
| | GFNKYYADSVKGRFTISRDNSKNTLYLQMNSLRAEDTAVYYCGGSGYALHDDYY | |
| | GQDVWGQGTLVTVSS | |
| H2/L2-18 | QAQLQGSGGGWQPGRSLRLSCAASGFTFSSYGMHWVRQAPGKGLEWVAVISYKG | 1293 |
| | SHKYYADSVKGRFTISRDNSKNTLYLQMNSLRAEDTAVYYCGGSGYALHDDYYG | |
| | LDVWGQGTLVTVSS | |
| H2/L2-47 | QVQLQSSEGGVVQPGRSLRLSCAASGFTFSSYGMHWVRQAPGKGLEWVAVISYK | 1289 |
| | GSNKYYADSVKGRFTISRDNSKNTLYLQMNSLRAEDTAVYYCGGSGYALHDDYY | |
| | GLDVWGQGTLVTVSS | |
| H2/L2-20 | QAQLQSSGGGVVQPGRSLRLSCAASGFTVSSYGMHWVRQAPGKGLEWVAVISYT | 1294 |
| | GSNKYYADSVKGRFTISRDNSKNTLYLQMNSLRAEDTAVYYCGGSGYALHDDYY | |
| | GLDVWGQGTLVTVSS | |
| H2/L2-80 | QVQLQSSGGGVVQPGRSLRLSCAASGFTFSSYGMHWVRQAPGKGLEWVAVISYT | 1295 |
| | GSNKYYADSVKGRFTISRDNSKNTLYLQMNSLRAEDTAVYYCGGSGYALHDDYY | |
| | GLDVWGQGTLVTVSS | |
| H2/L2-83 | QAQLQGSGGGWQPGRSLRLSCAASGFTFSSYGMHWVRQAPGKGLEWVAVISYKG | 1296 |
| | SNKYYADSVKGRFTISRDNSKNTLYLQMNSLRAEDTAVYYCGGSGYALHDDYYG | |
| | LDVWGQGTLVTVSS | |
| H3-1 | QVQLQGSGGGWQPGRSLRLSCAASGFTFSSYGMHWVRQAPGKGLEWVAVISYDD | 1297 |
| | AHKYYADSVKGRFTISRDNSKNTLYLQMNSLRAEDTAVYYCGGSGYALHDQYKP | |
| | VDVWGQGTLVTVSS | |
| H3-2 | QAQLQESEGGVVQPGGSLRLSCAASGFTFSSYGMHWVRQAPGKGLEWVAVISYN | 1298 |
| | DLNKYYADSVKGRFTISRDNSKNTLYLQMNSLRAEDTAVYYCGGSGYALHDFQD | |
| | PTDVWGQGTLVTVSS | |
| H3-3 | QVQLQSSGGGVVQPGRSLRLSCAASGFTVSSYGMHWVRQAPGKGLEWVAVISYS | 1299 |
| | GSNKYYADSVKGRFTISRDNSKNTLYLQMNSLRAEDTAVYYCGGSGYALHDQYK | |
| | PVDVWGQGTLVTVSS | |
| H3-4 | QVQLQGSGGGWQPGRSLRLSCAASGFTFSSYGMHWVRQAPGKGLEWVAVISYDD | 1297 |
| | AHKYYADSVKGRFTISRDNSKNTLYLQMNSLRAEDTAVYYCGGSGYALHDQYKP | |
| | VDVWGQGTLVTVSS | |
| H3-5 | QVQLQGSGGGWQPGRSLRLSCAASGFTFSSYGMHWVRQAPGKGLEWVAVISYTG | 1300 |
| | ANKYYADSVKGRFTISRDNSKNTLYLQMNSLRAEDTAVYYCGGSGYNLHDDYYG | |
| | LDVWGQGTLVTVSS | |
| H3-6 | QAQLQRSGGGVVQPGRSLRLSCAASGFTFSSYGMHWVRQAPGKGLEWVAVISYD | 1301 |
| | DAHKYYADSVKGRFTISRDNSKNTLYLQMNSLRAEDTAVYYCGGSGYALHDQYK | |
| | PVDVWGQGTLVTVSS | |
| H3-7 | QVQLQSSEGGVVQPGRSLRLSCAASGFTLSSYGMHWVRQAPGKGLEWVAVISYT | 1302 |
| | GSNKYYADSVKGRFTISRDNSKNTLYLQMNSLRAEDTAVYYCGGSGYEFHEDYY | |
| | GLDVWGQGTLVTVSS | |
| H3-8 | QAQLQGSEGGVVQPGRSLRLSCAASGFTFSSYGMHWVRQAPGKGLEWVAVISYD | 1303 |
| | DAHKYYADSVKGRFTISRDNSKNTLYLQMNSLRAEDTAVYYCGGSGYALHDQYK | |
| | PVDVWGQGTLVTVSS | |
| H3-9 | QVQLQGSGGGWQPGRSLRLSCAASGFTFSSYGMHWVRQAPGKGLEWVAVISYDD | 1297 |
| | AHKYYADSVKGRFTISRDNSKNTLYLQMNSLRAEDTAVYYCGGSGYALHDQYKP | |
| | VDVWGQGTLVTVSS | |
| H3-10 | QVQLQSSGGGVVQPGRSLRLSCAASGFTFSSYGMHWVRQAPGKGLEWVAVISYN | 1304 |
| | DLNKYYADSVKGRFTISRDNSKNTLYLQMNSLRAEDTAVYYCGGSGYEFQGDYY | |
| | GLDVWGQGTLVTVSS | |
| H3-11 | QVQLQSSEGGVVQPGRSLRLSCAASGFTFSSYGMHWVRQAPGKGLEWVAVISYN | 1305 |
| | DANKYYADSVKGRFTISRDNSKNTLYLQMNSLRAEDTAVYYCGGSGYELRDDYY | |
| | GLDVWGQGTLVTVSS | |
| H3-12 | QAQLQSSEGGVVQPGRSLRLSCAASGFTFSSYGMHWVRQAPGKGLEWVAVISYD | 1306 |
| | ESNKYYADSVKGRFTISRDNSKNTLYLQMNSLRAEDTAVYYCGGSGYEVDQDYY | |
| | GLDVWGQGTLVTVSS | |
| H3-13 | QVQLQESGGGVVQPGRSLRLSCAASGFTFSSYGMHWVRQAPGKGLEWVAVISYD | 1307 |
| | DAHKYYADSVKGRFTISRDNSKNTLYLQMNSLRAEDTAVYYCGGSGYALHDQYK | |
| | PVDVWGQGTLVTVSS | |
| H3-14 | QVQLQGSGGGVVQPGRSLRLSCAASGFTFSSYGMHWVRQAPGKGLEWVAVISYD | 1297 |
| | DAHKYYADSVKGRFTISRDNSKNTLYLQMNSLRAEDTAVYYCGGSGYALHDQYK | |
| | PVDVWGQGTLVTVSS | |
| H3-15 | QVQLQGSGGGVVQPGRSLRLSCAASGFTVSSYGMHWVRQAPGKGLEWVAVISYD | 1308 |
| | DANKYYADSVKGRFTISRDSSKNTLYLQMNSLRAEDTAVYYCGGSGYAYDGDYY | |
| | GLDVWGQGTLVTVSS | |

TABLE 15P
BCMA Binders - scFv sequences

| | | SEQ |
|---|---|---|
| | | ID |
| Binder | Sequence | NO: |
| H2/L2-88 | QVQLQSSEGGVVQPGRSLRLSCAASGFTFSSYGMHWVRQAPGKGLEWVAVISY | 1309 |
| | KGSNKYYADSVKGRFTISRDNSKNTLYLQMNSLRAEDTAVYYCGGSGYALHDD | |
| | YYGLDVWGQGTLVTVSSSGGGGSGGGGSGGGGSGGGGSQSALTQPASVSGSPG | |
| | QSITISCTGTSSDVGGYNYVSWYQQHPGKAPKLMIYEVSNRLRGVSNRFSGSK | |
| | FGNTASLTISGLQAEDEADYYCSSYTSSSALYVFGSGTKVTVL | |
| H2/L2-36 | QAQLQSSGGGVVQPGRSLRLSCAASGFTFSSYGMHWVRQAPGKGLEWVAVISY | 1310 |
| | KGSNKYYADSVKGRFTISRDNSKNTLYLQMNSLRAEDTAVYYCGGSGYALHDD | |
| | YYGLDVWGQGTLVTVSSSGGGGSGGGGSGGGGSGGGGSQSALTQPASVSGSPG | |
| | QSITISCTGTSSDVGGYNYVSWYQQHPGKAPKLMIYEVSNRLRGVSNRFSGSK | |
| | FGNTASLTISGLQAEDEADYYCSSYTSSSTLYVFGSGTKVTVL | |
| H2/L2-34 | QVQLQDSEGGVVQPGRSLRLSCAASGFTFSSYGMHWVRQAPGKGLEWVAVISY | 1311 |
| | TGTKKYYADSVKGRFTISRDNSKNTLYLQMNSLRAEDTAVYYCGGSGYALHDD | |
| | YYGLDVWGQGTLVTVSSSGGGGSGGGGSGGGGSGGGGSQSALTQPASVSGSPG | |
| | QSITISCTGTSSDVGGYNYVSWYQQHPGKAPKLMIYDVSNRPWGVSNRFSGSK | |
| | FGNTASLTISGLQAEDEADYYCSSYTSSSALYVFGSGTKVTVM | |
| H2/L2-68 | QAQLQSSEGGVVQPGRSLRLSCAASGFTFSSYGMHWVRQAPGKGLEWVAVISY | 1312 |
| | RGFNKYYADSVKGRFTISRDNSKNTLYLQMNSLRAEDTAVYYCGGSGYALHDD | |
| | YYGQDVWGQGTLVTVSSSGGGGSGGGGSGGGGSGGGGSQSALTQPASVSGSPG | |
| | QSITISCTGTSSDVGGYNYVSWYQQHPGKAPKLMIYDVSNRLSGVSNRFSGSK | |
| | FGNTASLTISGLQAEDEADYYCSSYTSSSTLYVFGSGTKVTVL | |
| H2/L2-18 | QAQLQGSGGGVVQPGRSLRLSCAASGFTFSSYGMHWVRQAPGKGLEWVAVISY | 1313 |
| | KGSHKYYADSVKGRFTISRDNSKNTLYLQMNSLRAEDTAVYYCGGSGYALHDD | |
| | YYGLDVWGQGTLVTVSSSGGGGSGGGGSGGGGSGGGGSQSALTQPASVSGSPG | |
| | QSITISCTGTSSDVGGYNYVSWYQQHPGKAPKLMIYDVSNRPWGVSNRFSGSK | |
| | FGNTASLTISGLQAEDEADYYCSSYTSSSTLYVFGSGTKVTVL | |
| H2/L2-47 | QVQLQSSEGGVVQPGRSLRLSCAASGFTFSSYGMHWVRQAPGKGLEWVAVISY | 1314 |
| | KGSNKYYADSVKGRFTISRDNSKNTLYLQMNSLRAEDTAVYYCGGSGYALHDD | |
| | YYGLDVWGQGTLVTVSSSGGGGSGGGGSGGGGSGGGGSQSALTQPASVSGSPG | |
| | QSITISCTGTSSDVGGYNYVSWYQQHPGKAPKLMIYDVSNRPWGVSNRFSGSK | |
| | FGNTASLTISGLQAEDEADYYCSSYTSSSTLYVFGSGTKVTVL | |
| H2/L2-20 | QAQLQSSGGGVVQPGRSLRLSCAASGFTVSSYGMHWVRQAPGKGLEWVAVISY | 1315 |
| | TGSNKYYADSVKGRFTISRDNSKNTLYLQMNSLRAEDTAVYYCGGSGYALHDD | |
| | YYGLDVWGQGTLVTVSSSGGGGSGGGGSGGGGSGGGGSQSALTQPASVSGSPG | |
| | QSITISCTGTSSDVGGYNYVSWYQQHPGKAPKLMIYDVSNRLRGVSNRFSGSK | |
| | FGNTASLTISGLQAEDEADYYCSSYTSSSALYVFGSGTKVTVL | |
| H2/L2-80 | QVQLQSSGGGVVQPGRSLRLSCAASGFTFSSYGMHWVRQAPGKGLEWVAVISY | 1316 |
| | TGSNKYYADSVKGRFTISRDNSKNTLYLQMNSLRAEDTAVYYCGGSGYALHDD | |
| | YYGLDVWGQGTLVTVSSSGGGGSGGGGSGGGGSGGGGSQSALTQPASVSGSPG | |
| | QSITISCTGTSSDVGGYNYVSWYQQHPGKAPKLMIYDVSNRAWGVSNRFSGSK | |
| | FGNTASLTISGLQAEDEADYYCSSYTSSSALYVFGSGTKVTVL | |
| H2/L2-83 | QAQLQGSGGGVVQPGRSLRLSCAASGFTFSSYGMHWVRQAPGKGLEWVAVISY | 1317 |
| | KGSNKYYADSVKGRFTISRDNSKNTLYLQMNSLRAEDTAVYYCGGSGYALHDD | |
| | YYGLDVWGQGTLVTVSSSGGGGSGGGGSGGGGSGGGGSQSALTQPASVSGSPG | |
| | QSITISCTGTSSDVGGYNYVSWYQQHPGKAPKLMIYEVSNRLRGVSNRFSGSK | |
| | FGNTASLTISGLQAEDEADYYCSSYTSSSTLYVFGSGTKVTVL | |

Tables 15A-1 to 15B-2 list CDR consensus sequences derived from the CDR sequences of the exemplary BCMA binding molecules described in Example 1. The CDR consensus sequences include sequences based upon the Kabat CDR sequences of the exemplary BCMA binding molecules, the Chothia CDR sequences of the exemplary BCMA binding molecules, the IMGT CDR sequences of the exemplary BCMA binding molecules, a combination of the Kabat and Chothia CDR sequences of the exemplary BCMA binding molecules, a combination of the Kabat and IMGT CDR sequences of the exemplary BCMA binding molecules, and a combination of the Chothia and IMGT CDR sequences of the exemplary BCMA binding molecules. The specific CDR sequences of the exemplary BCMA binding molecules described in the Examples are listed in Tables 15C1-15N-2. Exemplary VL and VH sequences are listed in Tables 15O-1 and 15O-2, respectively. Exemplary scFv sequences are listed in Table 15P.

In some embodiments, a BCMA ABM (e.g., ABM2 or ABM5) comprises a light chain CDR having an amino acid sequence of any one of the CDR consensus sequences listed in Table 15A-1 or Table 15B-1. In particular embodiments, the present disclosure MBMs comprising (or alternatively, consisting of) one, two, three, or more light chain CDRs selected the light chain CDRs described in Table 15A-1 or Table 15B-1.

In some embodiments, a BCMA ABM comprises a heavy chain CDR having an amino acid sequence of any one of the heavy chain CDRs listed in Table 15A-2 or Table 15B-2. In particular embodiments, the present disclosure provides BCMA ABMs comprising (or alternatively, consisting of) one, two, three, or more heavy chain CDRs selected the heavy chain CDRs described in Table 15A-2 or Table 15B-2.

In some embodiments, a BCMA ABM comprises CDR-L1, CDR-L2, CDR-L3, CDR-H1, CDR-H2 and CDR-H3 sequences of BCMA C1 as set forth in Tables 15A-1 and 15A-2. In some embodiments, a BCMA ABM comprises CDR-L1, CDR-L2, CDR-L3, CDR-H1, CDR-H2 and CDR-H3 sequences of BCMA C2 as set forth in Tables 15A-1 and 15A-2. In some embodiments, a BCMA ABM comprises CDR-L1, CDR-L2, CDR-L3, CDR-H1, CDR-H2 and CDR-H3 sequences of BCMA C3 as set forth in Tables 15A-1 and 15A-2. In some embodiments, a BCMA ABM comprises CDR-L1, CDR-L2, CDR-L3, CDR-H1, CDR-H2 and CDR-H3 sequences of BCMA C4 as set forth in Tables 15A-1 and 15A-2. In some embodiments, a BCMA ABM comprises CDR-L1, CDR-L2, CDR-L3, CDR-H1, CDR-H2 and CDR-H3 sequences of BCMA C5 as set forth in Tables 15A-1 and 15A-2. In some embodiments, a BCMA ABM comprises CDR-L1, CDR-L2, CDR-L3, CDR-H1, CDR-H2 and CDR-H3 sequences of BCMA C6 as set forth in Tables 15A-1 and 15A-2. In some embodiments, a BCMA ABM comprises CDR-L1, CDR-L2, CDR-L3, CDR-H1, CDR-H2 and CDR-H3 sequences of BCMA C7 as set forth in Tables 15A-1 and 15A-2. In some embodiments, a BCMA ABM comprises CDR-L1, CDR-L2, CDR-L3, CDR-H1, CDR-H2 and CDR-H3 sequences of BCMA C8 as set forth in Tables 15A-1 and 15A-2. In some embodiments, a BCMA ABM comprises CDR-L1, CDR-L2, CDR-L3, CDR-H1, CDR-H2 and CDR-H3 sequences of BCMA C9 as set forth in Tables 15A-1 and 15A-2. In some embodiments, a BCMA ABM comprises CDR-L1, CDR-L2, CDR-L3, CDR-H1, CDR-H2 and CDR-H3 sequences of BCMA 010 as set forth in Tables 15A-1 and 15A-2. In some embodiments, a BCMA ABM comprises CDR-L1, CDR-L2, CDR-L3, CDR-H1, CDR-H2 and CDR-H3 sequences of BCMA 011 as set forth in Tables 15A-1 and 15A-2. In some embodiments, a BCMA ABM comprises CDR-L1, CDR-L2, CDR-L3, CDR-H1, CDR-H2 and CDR-H3 sequences of BCMA C12 as set forth in Tables 15A-1 and 15A-2.

In some embodiments, a BCMA ABM comprises CDR-L1, CDR-L2, CDR-L3, CDR-H1, CDR-H2 and CDR-H3 sequences of BCMA C13 as set forth in Tables 15B-1 and 15B-2. In some embodiments, a BCMA ABM comprises CDR-L1, CDR-L2, CDR-L3, CDR-H1, CDR-H2 and CDR-H3 sequences of BCMA C14 as set forth in Tables 15B-1 and 15B-2. In some embodiments, a BCMA ABM comprises CDR-L1, CDR-L2, CDR-L3, CDR-H1, CDR-H2 and CDR-H3 sequences of BCMA C15 as set forth in Tables 15B-1 and 15B-2. In some embodiments, a BCMA ABM comprises CDR-L1, CDR-L2, CDR-L3, CDR-H1, CDR-H2 and CDR-H3 sequences of BCMA C16 as set forth in Tables 15B-1 and 15B-2. In some embodiments, a BCMA ABM comprises CDR-L1, CDR-L2, CDR-L3, CDR-H1, CDR-H2 and CDR-H3 sequences of BCMA C17 as set forth in Tables 15B-1 and 15B-2. In some embodiments, a BCMA ABM comprises CDR-L1, CDR-L2, CDR-L3, CDR-H1, CDR-H2 and CDR-H3 sequences of BCMA C18 as set forth in Tables 15B-1 and 15B-2. In some embodiments, a BCMA ABM comprises CDR-L1, CDR-L2, CDR-L3, CDR-H1, CDR-H2 and CDR-H3 sequences of BCMA C19 as set forth in Tables 15B-1 and 15B-2. In some embodiments, a BCMA ABM comprises CDR-L1, CDR-L2, CDR-L3, CDR-H1, CDR-H2 and CDR-H3 sequences of BCMA C20 as set forth in Tables 15B-1 and 15B-2. In some embodiments, a BCMA ABM comprises CDR-L1, CDR-L2, CDR-L3, CDR-H1, CDR-H2 and CDR-H3 sequences of BCMA C21 as set forth in Tables 15B-1 and 15B-2. In some embodiments, a BCMA ABM comprises CDR-L1, CDR-L2, CDR-L3, CDR-H1, CDR-H2 and CDR-H3 sequences of BCMA C22 as set forth in Tables 15B-1 and 15B-2. In some embodiments, a BCMA ABM comprises CDR-L1, CDR-L2, CDR-L3, CDR-H1, CDR-H2 and CDR-H3 sequences of BCMA C23 as set forth in Tables 15B-1 and 15B-2. In some embodiments, a BCMA ABM comprises CDR-L1, CDR-L2, CDR-L3, CDR-H1, CDR-H2 and CDR-H3 sequences of BCMA C24 as set forth in Tables 15B-1 and 15B-2. In some embodiments, a BCMA ABM comprises CDR-L1, CDR-L2, CDR-L3, CDR-H1, CDR-H2 and CDR-H3 sequences of BCMA C25 as set forth in Tables 15B-1 and 15B-2. In some embodiments, a BCMA ABM comprises CDR-L1, CDR-L2, CDR-L3, CDR-H1, CDR-H2 and CDR-H3 sequences of BCMA C26 as set forth in Tables 15B-1 and 15B-2. In some embodiments, a BCMA ABM comprises CDR-L1, CDR-L2, CDR-L3, CDR-H1, CDR-H2 and CDR-H3 sequences of BCMA C27 as set forth in Tables 15B-1 and 15B-2. In some embodiments, a BCMA ABM comprises CDR-L1, CDR-L2, CDR-L3, CDR-H1, CDR-H2 and CDR-H3 sequences of BCMA C28 as set forth in Tables 15B-1 and 15B-2.

In some embodiments, the BCMA ABM comprises a light chain CDR having an amino acid sequence of any one of the CDRs listed in Table 15C-1, Table 15D-1, Table 15E-1, Table 15F-1, Table 15G-1, Table 15H-1, Table 15I-1, Table 15J-1, Table 15K-1(a), Table 15K-1(b), Table 15L-1, Table 15M-1, Table 15N-1(a) or Table 15N-1(b). In particular embodiments, the present disclosure provides BCMA ABMs, comprising (or alternatively, consisting of) one, two, three, or more light chain CDRs selected the light chain CDRs described in Table 15C-1, Table 15D-1, Table 15E-1, Table 15F-1, Table 15G-1, Table 15H-1, Table 15I-1, Table 15J-1, Table 15K-1(a), Table 15K-1(b), Table 15L-1, Table 15M-1, Table 15N-1(a) and Table 15N-1(b).

In some embodiments, the BCMA ABM comprises a heavy chain CDR having an amino acid sequence of any one of the heavy chain CDRs listed in Table 15C-2, Table 15D-2, Table 15E-2, Table 15F-2, Table 15G-2, Table 15H-2, Table 15I-2, Table 15J-2, Table 15K-2, Table 15L-2, Table 15M-2, or Table 15N-2. In particular embodiments, the present disclosure provides BCMA ABMs, comprising (or alternatively, consisting of) one, two, three, or more heavy chain CDRs selected the heavy chain CDRs described in Table 15C-2, Table 15D-2, Table 15E-2, Table 15F-2, Table 15G-2, Table 15H-2, Table 15I-2, Table 15J-2, Table 15K-2, Table 15L-2, Table 15M-2, and Table 15N-2.

In some embodiments, the BCMA ABM comprises a VL domain having an amino acid sequence of any VL domain described in Table 15O-1. Other BCMA ABMs can include amino acids that have been mutated, yet have at least 80, 85, 90, 95, 96, 97, 98, or 99 percent identity in the VL domain with the VL domains depicted in the sequences described in Table 15O-1.

In some embodiments, the BCMA ABM comprises a VH domain having an amino acid sequence of any VH domain described in Table 15O-2. Other BCMA ABMs can include amino acids that have been mutated, yet have at least 80, 85, 90, 95, 96, 97, 98, or 99 percent identity in the VH domain with the VH domains depicted in the sequences described in Table 15O-2.

Other BCMA ABMs include amino acids that have been mutated, yet have at least 80, 85, 90, 95, 96, 97, 98, or 99 percent identity in the CDR regions with the CDR sequences described in Table 15. In some embodiments, such BCMA ABMs include mutant amino acid sequences where no more than 1, 2, 3, 4 or 5 amino acids have been mutated in the CDR regions when compared with the CDR sequences described in Table 15.

Other BCMA ABMs include VH and/or VL domains comprising amino acid sequences having at least 80, 85, 90, 95, 96, 97, 98, or 99 percent identity to the VH and/or VL sequences described in Table 15. In some embodiments, BCMA ABMs include VH and/or VL domains where no more than 1, 2, 3, 4 or 5 amino acids have been mutated when compared with the VH and/or VL domains depicted in the sequences described in Table 15, while retaining substantially the same therapeutic activity.

VH and VL sequences (amino acid sequences and the nucleotide sequences encoding the amino acid sequences) can be “mixed and matched” to create other BCMA ABMs. Such “mixed and matched” BCMA ABMs can be tested using known binding assays (e.g., ELISAs, assays described in the Examples). When chains are mixed and matched, a VH sequence from a particular VH/VL pairing should be replaced with a structurally similar VH sequence. A VL sequence from a particular VH/VL pairing should be replaced with a structurally similar VL sequence.

Accordingly, in one embodiment, the present disclosure provides BCMA ABMs having: a heavy chain variable region (VH) comprising an amino acid sequence selected from any one of the VH sequences described in Table 15-02; and a light chain variable region (VL) comprising an amino acid sequence described in Table 15-01.

In another embodiment, the present disclosure provides BCMA ABMs that comprise the CDR-H1, CDR-H2, CDR-H3, CDR-L1, CDR-L2, and CDR-L3 as described in Table 15, or any combination thereof.

In some embodiments, a BCMA ABM comprises CDR-L1, CDR-L2, CDR-L3, CDR-H1, CDR-H2 and CDR-H3 sequences of AB1 as set forth in Tables 15C-1 and 15C-2. In some embodiments, a BCMA ABM comprises CDR-L1, CDR-L2, CDR-L3, CDR-H1, CDR-H2 and CDR-H3 sequences of AB1 as set forth in Tables 15D-1 and 15D-2. In some embodiments, a BCMA ABM comprises CDR-L1, CDR-L2, CDR-L3, CDR-H1, CDR-H2 and CDR-H3 sequences of AB1 as set forth in Tables 15E-1 and 15E-2. In some embodiments, a BCMA ABM comprises CDR-L1, CDR-L2, CDR-L3, CDR-H1, CDR-H2 and CDR-H3 sequences of AB1 as set forth in Tables 15F-1 and 15F-2. In some embodiments, a BCMA ABM comprises CDR-L1, CDR-L2, CDR-L3, CDR-H1, CDR-H2 and CDR-H3 sequences of AB1 as set forth in Tables 15G-1 and 15G-2. In some embodiments, a BCMA ABM comprises CDR-L1, CDR-L2, CDR-L3, CDR-H1, CDR-H2 and CDR-H3 sequences of AB1 as set forth in Tables 15H-1 and 15H-2.

In some embodiments, a BCMA ABM comprises CDR-L1, CDR-L2, CDR-L3, CDR-H1, CDR-H2 and CDR-H3 sequences of AB2 as set forth in Tables 15C-1 and 15C-2. In some embodiments, a BCMA ABM comprises CDR-L1, CDR-L2, CDR-L3, CDR-H1, CDR-H2 and CDR-H3 sequences of AB2 as set forth in Tables 15D-1 and 15D-2. In some embodiments, a BCMA ABM comprises CDR-L1, CDR-L2, CDR-L3, CDR-H1, CDR-H2 and CDR-H3 sequences of AB2 as set forth in Tables 15E-1 and 15E-2. In some embodiments, a BCMA ABM comprises CDR-L1, CDR-L2, CDR-L3, CDR-H1, CDR-H2 and CDR-H3 sequences of AB2 as set forth in Tables 15F-1 and 15F-2. In some embodiments, a BCMA ABM comprises CDR-L1, CDR-L2, CDR-L3, CDR-H1, CDR-H2 and CDR-H3 sequences of AB2 as set forth in Tables 15G-1 and 15G-2. In some embodiments, a BCMA ABM comprises CDR-L1, CDR-L2, CDR-L3, CDR-H1, CDR-H2 and CDR-H3 sequences of AB2 as set forth in Tables 15H-1 and 15H-2.

In some embodiments, a BCMA ABM comprises CDR-L1, CDR-L2, CDR-L3, CDR-H1, CDR-H2 and CDR-H3 sequences of R1F2 as set forth in Tables 15C-1 and 15C-2. In some embodiments, a BCMA ABM comprises CDR-L1, CDR-L2, CDR-L3, CDR-H1, CDR-H2 and CDR-H3 sequences of R1F2 as set forth in Tables 15D-1 and 15D-2. In some embodiments, a BCMA ABM comprises CDR-L1, CDR-L2, CDR-L3, CDR-H1, CDR-H2 and CDR-H3 sequences of R1F2 as set forth in Tables 15E-1 and 15E-2. In some embodiments, a BCMA ABM comprises CDR-L1, CDR-L2, CDR-L3, CDR-H1, CDR-H2 and CDR-H3 sequences of R1F2 as set forth in Tables 15F-1 and 15F-2. In some embodiments, a BCMA ABM comprises CDR-L1, CDR-L2, CDR-L3, CDR-H1, CDR-H2 and CDR-H3 sequences of R1F2 as set forth in Tables 15G-1 and 15G-2. In some embodiments, a BCMA ABM comprises CDR-L1, CDR-L2, CDR-L3, CDR-H1, CDR-H2 and CDR-H3 sequences of R1F2 as set forth in Tables 15H-1 and 15H-2.

In some embodiments, a BCMA ABM comprises CDR-L1, CDR-L2, CDR-L3, CDR-H1, CDR-H2 and CDR-H3 sequences of PALF03 as set forth in Tables 15C-1 and 15C-2. In some embodiments, a BCMA ABM comprises CDR-L1, CDR-L2, CDR-L3, CDR-H1, CDR-H2 and CDR-H3 sequences of PALF03 as set forth in Tables 15D-1 and 15D-2. In some embodiments, a BCMA ABM comprises CDR-L1, CDR-L2, CDR-L3, CDR-H1, CDR-H2 and CDR-H3 sequences of PALF03 as set forth in Tables 15E-1 and 15E-2. In some embodiments, a BCMA ABM comprises CDR-L1, CDR-L2, CDR-L3, CDR-H1, CDR-H2 and CDR-H3 sequences of PALF03 as set forth in Tables 15F-1 and 15F-2. In some embodiments, a BCMA ABM comprises CDR-L1, CDR-L2, CDR-L3, CDR-H1, CDR-H2 and CDR-H3 sequences of PALF03 as set forth in Tables 15G-1 and 15G-2. In some embodiments, a BCMA ABM comprises CDR-L1, CDR-L2, CDR-L3, CDR-H1, CDR-H2 and CDR-H3 sequences of PALF03 as set forth in Tables 15H-1 and 15H-2.

In some embodiments, a BCMA ABM comprises CDR-L1, CDR-L2, CDR-L3, CDR-H1, CDR-H2 and CDR-H3 sequences of PALF04 as set forth in Tables 15C-1 and 15C-2. In some embodiments, a BCMA ABM comprises CDR-L1, CDR-L2, CDR-L3, CDR-H1, CDR-H2 and CDR-H3 sequences of PALF04 as set forth in Tables 15D-1 and 15D-2. In some embodiments, a BCMA ABM comprises CDR-L1, CDR-L2, CDR-L3, CDR-H1, CDR-H2 and CDR-H3 sequences of PALF04 as set forth in Tables 15E-1 and 15E-2. In some embodiments, a BCMA ABM comprises CDR-L1, CDR-L2, CDR-L3, CDR-H1, CDR-H2 and CDR-H3 sequences of PALF04 as set forth in Tables 15F-1 and 15F-2. In some embodiments, a BCMA ABM comprises CDR-L1, CDR-L2, CDR-L3, CDR-H1, CDR-H2 and CDR-H3 sequences of PALF04 as set forth in Tables 15G-1 and 15G-2. In some embodiments, a BCMA ABM comprises CDR-L1, CDR-L2, CDR-L3, CDR-H1, CDR-H2 and CDR-H3 sequences of PALF04 as set forth in Tables 15H-1 and 15H-2.

In some embodiments, a BCMA ABM comprises CDR-L1, CDR-L2, CDR-L3, CDR-H1, CDR-H2 and CDR-H3 sequences of PALF05 as set forth in Tables 15C-1 and 15C-2. In some embodiments, a BCMA ABM comprises CDR-L1, CDR-L2, CDR-L3, CDR-H1, CDR-H2 and CDR-H3 sequences of PALF05 as set forth in Tables 15D-1 and 15D-2. In some embodiments, a BCMA ABM comprises CDR-L1, CDR-L2, CDR-L3, CDR-H1, CDR-H2 and CDR-H3 sequences of PALF05 as set forth in Tables 15E-1 and 15E-2. In some embodiments, a BCMA ABM comprises CDR-L1, CDR-L2, CDR-L3, CDR-H1, CDR-H2 and CDR-H3 sequences of PALF05 as set forth in Tables 15F-1 and 15F-2. In some embodiments, a BCMA ABM comprises CDR-L1, CDR-L2, CDR-L3, CDR-H1, CDR-H2 and CDR-H3 sequences of PALF05 as set forth in Tables 15G-1 and 15G-2. In some embodiments, a BCMA ABM comprises CDR-L1, CDR-L2, CDR-L3, CDR-H1, CDR-H2 and CDR-H3 sequences of PALF05 as set forth in Tables 15H-1 and 15H-2.

In some embodiments, a BCMA ABM comprises CDR-L1, CDR-L2, CDR-L3, CDR-H1, CDR-H2 and CDR-H3 sequences of PALF06 as set forth in Tables 15C-1 and 15C-2. In some embodiments, a BCMA ABM comprises CDR-L1, CDR-L2, CDR-L3, CDR-H1, CDR-H2 and CDR-H3 sequences of PALF06 as set forth in Tables 15D-1 and 15D-2. In some embodiments, a BCMA ABM comprises CDR-L1, CDR-L2, CDR-L3, CDR-H1, CDR-H2 and CDR-H3 sequences of PALF06 as set forth in Tables 15E-1 and 15E-2. In some embodiments, a BCMA ABM comprises CDR-L1, CDR-L2, CDR-L3, CDR-H1, CDR-H2 and CDR-H3 sequences of PALF06 as set forth in Tables 15F-1 and 15F-2. In some embodiments, a BCMA ABM comprises CDR-L1, CDR-L2, CDR-L3, CDR-H1, CDR-H2 and CDR-H3 sequences of PALF06 as set forth in Tables 15G-1 and 15G-2. In some embodiments, a BCMA ABM comprises CDR-L1, CDR-L2, CDR-L3, CDR-H1, CDR-H2 and CDR-H3 sequences of PALF06 as set forth in Tables 15H-1 and 15H-2.

In some embodiments, a BCMA ABM comprises CDR-L1, CDR-L2, CDR-L3, CDR-H1, CDR-H2 and CDR-H3 sequences of PALF07 as set forth in Tables 15C-1 and 15C-2. In some embodiments, a BCMA ABM comprises CDR-L1, CDR-L2, CDR-L3, CDR-H1, CDR-H2 and CDR-H3 sequences of PALF07 as set forth in Tables 15D-1 and 15D-2. In some embodiments, a BCMA ABM comprises CDR-L1, CDR-L2, CDR-L3, CDR-H1, CDR-H2 and CDR-H3 sequences of PALF07 as set forth in Tables 15E-1 and 15E-2. In some embodiments, a BCMA ABM comprises CDR-L1, CDR-L2, CDR-L3, CDR-H1, CDR-H2 and CDR-H3 sequences of PALF07 as set forth in Tables 15F-1 and 15F-2. In some embodiments, a BCMA ABM comprises CDR-L1, CDR-L2, CDR-L3, CDR-H1, CDR-H2 and CDR-H3 sequences of PALF07 as set forth in Tables 15G-1 and 15G-2. In some embodiments, a BCMA ABM comprises CDR-L1, CDR-L2, CDR-L3, CDR-H1, CDR-H2 and CDR-H3 sequences of PALF07 as set forth in Tables 15H-1 and 15H-2.

In some embodiments, a BCMA ABM comprises CDR-L1, CDR-L2, CDR-L3, CDR-H1, CDR-H2 and CDR-H3 sequences of PALF08 as set forth in Tables 15C-1 and 15C-2. In some embodiments, a BCMA ABM comprises CDR-L1, CDR-L2, CDR-L3, CDR-H1, CDR-H2 and CDR-H3 sequences of PALF08 as set forth in Tables 15D-1 and 15D-2. In some embodiments, a BCMA ABM comprises CDR-L1, CDR-L2, CDR-L3, CDR-H1, CDR-H2 and CDR-H3 sequences of PALF08 as set forth in Tables 15E-1 and 15E-2. In some embodiments, a BCMA ABM comprises CDR-L1, CDR-L2, CDR-L3, CDR-H1, CDR-H2 and CDR-H3 sequences of PALF08 as set forth in Tables 15F-1 and 15F-2. In some embodiments, a BCMA ABM comprises CDR-L1, CDR-L2, CDR-L3, CDR-H1, CDR-H2 and CDR-H3 sequences of PALF08 as set forth in Tables 15G-1 and 15G-2. In some embodiments, a BCMA ABM comprises CDR-L1, CDR-L2, CDR-L3, CDR-H1, CDR-H2 and CDR-H3 sequences of PALF08 as set forth in Tables 15H-1 and 15H-2.

In some embodiments, a BCMA ABM comprises CDR-L1, CDR-L2, CDR-L3, CDR-H1, CDR-H2 and CDR-H3 sequences of PALF09 as set forth in Tables 15C-1 and 15C-2. In some embodiments, a BCMA ABM comprises CDR-L1, CDR-L2, CDR-L3, CDR-H1, CDR-H2 and CDR-H3 sequences of PALF09 as set forth in Tables 15D-1 and 15D-2. In some embodiments, a BCMA ABM comprises CDR-L1, CDR-L2, CDR-L3, CDR-H1, CDR-H2 and CDR-H3 sequences of PALF09 as set forth in Tables 15E-1 and 15E-2. In some embodiments, a BCMA ABM comprises CDR-L1, CDR-L2, CDR-L3, CDR-H1, CDR-H2 and CDR-H3 sequences of PALF09 as set forth in Tables 15F-1 and 15F-2. In some embodiments, a BCMA ABM comprises CDR-L1, CDR-L2, CDR-L3, CDR-H1, CDR-H2 and CDR-H3 sequences of PALF09 as set forth in Tables 15G-1 and 15G-2. In some embodiments, a BCMA ABM comprises CDR-L1, CDR-L2, CDR-L3, CDR-H1, CDR-H2 and CDR-H3 sequences of PALF09 as set forth in Tables 15H-1 and 15H-2.

In some embodiments, a BCMA ABM comprises CDR-L1, CDR-L2, CDR-L3, CDR-H1, CDR-H2 and CDR-H3 sequences of PALF12 as set forth in Tables 15C-1 and 15C-2. In some embodiments, a BCMA ABM comprises CDR-L1, CDR-L2, CDR-L3, CDR-H1, CDR-H2 and CDR-H3 sequences of PALF12 as set forth in Tables 15D-1 and 15D-2. In some embodiments, a BCMA ABM comprises CDR-L1, CDR-L2, CDR-L3, CDR-H1, CDR-H2 and CDR-H3 sequences of PALF12 as set forth in Tables 15E-1 and 15E-2. In some embodiments, a BCMA ABM comprises CDR-L1, CDR-L2, CDR-L3, CDR-H1, CDR-H2 and CDR-H3 sequences of PALF12 as set forth in Tables 15F-1 and 15F-2. In some embodiments, a BCMA ABM comprises CDR-L1, CDR-L2, CDR-L3, CDR-H1, CDR-H2 and CDR-H3 sequences of PALF12 as set forth in Tables 15G-1 and 15G-2. In some embodiments, a BCMA ABM comprises CDR-L1, CDR-L2, CDR-L3, CDR-H1, CDR-H2 and CDR-H3 sequences of PALF12 as set forth in Tables 15H-1 and 15H-2.

In some embodiments, a BCMA ABM comprises CDR-L1, CDR-L2, CDR-L3, CDR-H1, CDR-H2 and CDR-H3 sequences of PALF13 as set forth in Tables 15C-1 and 15C-2. In some embodiments, a BCMA ABM comprises CDR-L1, CDR-L2, CDR-L3, CDR-H1, CDR-H2 and CDR-H3 sequences of PALF13 as set forth in Tables 15D-1 and 15D-2. In some embodiments, a BCMA ABM comprises CDR-L1, CDR-L2, CDR-L3, CDR-H1, CDR-H2 and CDR-H3 sequences of PALF13 as set forth in Tables 15E-1 and 15E-2. In some embodiments, a BCMA ABM comprises CDR-L1, CDR-L2, CDR-L3, CDR-H1, CDR-H2 and CDR-H3 sequences of PALF13 as set forth in Tables 15F-1 and 15F-2. In some embodiments, a BCMA ABM comprises CDR-L1, CDR-L2, CDR-L3, CDR-H1, CDR-H2 and CDR-H3 sequences of PALF13 as set forth in Tables 15G-1 and 15G-2. In some embodiments, a BCMA ABM comprises CDR-L1, CDR-L2, CDR-L3, CDR-H1, CDR-H2 and CDR-H3 sequences of PALF13 as set forth in Tables 15H-1 and 15H-2.

In some embodiments, a BCMA ABM comprises CDR-L1, CDR-L2, CDR-L3, CDR-H1, CDR-H2 and CDR-H3 sequences of PALF14 as set forth in Tables 15C-1 and 15C-2. In some embodiments, a BCMA ABM comprises CDR-L1, CDR-L2, CDR-L3, CDR-H1, CDR-H2 and CDR-H3 sequences of PALF14 as set forth in Tables 15D-1 and 15D-2. In some embodiments, a BCMA ABM comprises CDR-L1, CDR-L2, CDR-L3, CDR-H1, CDR-H2 and CDR-H3 sequences of PALF14 as set forth in Tables 15E-1 and 15E-2. In some embodiments, a BCMA ABM comprises CDR-L1, CDR-L2, CDR-L3, CDR-H1, CDR-H2 and CDR-H3 sequences of PALF14 as set forth in Tables 15F-1 and 15F-2. In some embodiments, a BCMA ABM comprises CDR-L1, CDR-L2, CDR-L3, CDR-H1, CDR-H2 and CDR-H3 sequences of PALF14 as set forth in Tables 15G-1 and 15G-2. In some embodiments, a BCMA ABM comprises CDR-L1, CDR-L2, CDR-L3, CDR-H1, CDR-H2 and CDR-H3 sequences of PALF14 as set forth in Tables 15H-1 and 15H-2.

In some embodiments, a BCMA ABM comprises CDR-L1, CDR-L2, CDR-L3, CDR-H1, CDR-H2 and CDR-H3 sequences of PALF15 as set forth in Tables 15C-1 and 15C-2. In some embodiments, a BCMA ABM comprises CDR-L1, CDR-L2, CDR-L3, CDR-H1, CDR-H2 and CDR-H3 sequences of PALF15 as set forth in Tables 15D-1 and 15D-2. In some embodiments, a BCMA ABM comprises CDR-L1, CDR-L2, CDR-L3, CDR-H1, CDR-H2 and CDR-H3 sequences of PALF15 as set forth in Tables 15E-1 and 15E-2. In some embodiments, a BCMA ABM comprises CDR-L1, CDR-L2, CDR-L3, CDR-H1, CDR-H2 and CDR-H3 sequences of PALF15 as set forth in Tables 15F-1 and 15F-2. In some embodiments, a BCMA ABM comprises CDR-L1, CDR-L2, CDR-L3, CDR-H1, CDR-H2 and CDR-H3 sequences of PALF15 as set forth in Tables 15G-1 and 15G-2. In some embodiments, a BCMA ABM comprises CDR-L1, CDR-L2, CDR-L3, CDR-H1, CDR-H2 and CDR-H3 sequences of PALF15 as set forth in Tables 15H-1 and 15H-2.

In some embodiments, a BCMA ABM comprises CDR-L1, CDR-L2, CDR-L3, CDR-H1, CDR-H2 and CDR-H3 sequences of PALF16 as set forth in Tables 15C-1 and 15C-2. In some embodiments, a BCMA ABM comprises CDR-L1, CDR-L2, CDR-L3, CDR-H1, CDR-H2 and CDR-H3 sequences of PALF16 as set forth in Tables 15D-1 and 15D-2. In some embodiments, a BCMA ABM comprises CDR-L1, CDR-L2, CDR-L3, CDR-H1, CDR-H2 and CDR-H3 sequences of PALF16 as set forth in Tables 15E-1 and 15E-2. In some embodiments, a BCMA ABM comprises CDR-L1, CDR-L2, CDR-L3, CDR-H1, CDR-H2 and CDR-H3 sequences of PALF16 as set forth in Tables 15F-1 and 15F-2. In some embodiments, a BCMA ABM comprises CDR-L1, CDR-L2, CDR-L3, CDR-H1, CDR-H2 and CDR-H3 sequences of PALF16 as set forth in Tables 15G-1 and 15G-2. In some embodiments, a BCMA ABM comprises CDR-L1, CDR-L2, CDR-L3, CDR-H1, CDR-H2 and CDR-H3 sequences of PALF16 as set forth in Tables 15H-1 and 15H-2.

In some embodiments, a BCMA ABM comprises CDR-L1, CDR-L2, CDR-L3, CDR-H1, CDR-H2 and CDR-H3 sequences of PALF17 as set forth in Tables 15C-1 and 15C-2. In some embodiments, a BCMA ABM comprises CDR-L1, CDR-L2, CDR-L3, CDR-H1, CDR-H2 and CDR-H3 sequences of PALF17 as set forth in Tables 15D-1 and 15D-2. In some embodiments, a BCMA ABM comprises CDR-L1, CDR-L2, CDR-L3, CDR-H1, CDR-H2 and CDR-H3 sequences of PALF17 as set forth in Tables 15E-1 and 15E-2. In some embodiments, a BCMA ABM comprises CDR-L1, CDR-L2, CDR-L3, CDR-H1, CDR-H2 and CDR-H3 sequences of PALF17 as set forth in Tables 15F-1 and 15F-2. In some embodiments, a BCMA ABM comprises CDR-L1, CDR-L2, CDR-L3, CDR-H1, CDR-H2 and CDR-H3 sequences of PALF17 as set forth in Tables 15G-1 and 15G-2. In some embodiments, a BCMA ABM comprises CDR-L1, CDR-L2, CDR-L3, CDR-H1, CDR-H2 and CDR-H3 sequences of PALF17 as set forth in Tables 15H-1 and 15H-2.

In some embodiments, a BCMA ABM comprises CDR-L1, CDR-L2, CDR-L3, CDR-H1, CDR-H2 and CDR-H3 sequences of PALF18 as set forth in Tables 15C-1 and 15C-2. In some embodiments, a BCMA ABM comprises CDR-L1, CDR-L2, CDR-L3, CDR-H1, CDR-H2 and CDR-H3 sequences of PALF18 as set forth in Tables 15D-1 and 15D-2. In some embodiments, a BCMA ABM comprises CDR-L1, CDR-L2, CDR-L3, CDR-H1, CDR-H2 and CDR-H3 sequences of PALF18 as set forth in Tables 15E-1 and 15E-2. In some embodiments, a BCMA ABM comprises CDR-L1, CDR-L2, CDR-L3, CDR-H1, CDR-H2 and CDR-H3 sequences of PALF18 as set forth in Tables 15F-1 and 15F-2. In some embodiments, a BCMA ABM comprises CDR-L1, CDR-L2, CDR-L3, CDR-H1, CDR-H2 and CDR-H3 sequences of PALF18 as set forth in Tables 15G-1 and 15G-2. In some embodiments, a BCMA ABM comprises CDR-L1, CDR-L2, CDR-L3, CDR-H1, CDR-H2 and CDR-H3 sequences of PALF18 as set forth in Tables 15H-1 and 15H-2.

In some embodiments, a BCMA ABM comprises CDR-L1, CDR-L2, CDR-L3, CDR-H1, CDR-H2 and CDR-H3 sequences of PALF19 as set forth in Tables 15C-1 and 15C-2. In some embodiments, a BCMA ABM comprises CDR-L1, CDR-L2, CDR-L3, CDR-H1, CDR-H2 and CDR-H3 sequences of PALF19 as set forth in Tables 15D-1 and 15D-2. In some embodiments, a BCMA ABM comprises CDR-L1, CDR-L2, CDR-L3, CDR-H1, CDR-H2 and CDR-H3 sequences of PALF19 as set forth in Tables 15E-1 and 15E-2. In some embodiments, a BCMA ABM comprises CDR-L1, CDR-L2, CDR-L3, CDR-H1, CDR-H2 and CDR-H3 sequences of PALF19 as set forth in Tables 15F-1 and 15F-2. In some embodiments, a BCMA ABM comprises CDR-L1, CDR-L2, CDR-L3, CDR-H1, CDR-H2 and CDR-H3 sequences of PALF19 as set forth in Tables 15G-1 and 15G-2. In some embodiments, a BCMA ABM comprises CDR-L1, CDR-L2, CDR-L3, CDR-H1, CDR-H2 and CDR-H3 sequences of PALF19 as set forth in Tables 15H-1 and 15H-2.

In some embodiments, a BCMA ABM comprises CDR-L1, CDR-L2, CDR-L3, CDR-H1, CDR-H2 and CDR-H3 sequences of PALF20 as set forth in Tables 15C-1 and 15C-2. In some embodiments, a BCMA ABM comprises CDR-L1, CDR-L2, CDR-L3, CDR-H1, CDR-H2 and CDR-H3 sequences of PALF20 as set forth in Tables 15D-1 and 15D-2. In some embodiments, a BCMA ABM comprises CDR-L1, CDR-L2, CDR-L3, CDR-H1, CDR-H2 and CDR-H3 sequences of PALF20 as set forth in Tables 15E-1 and 15E-2. In some embodiments, a BCMA ABM comprises CDR-L1, CDR-L2, CDR-L3, CDR-H1, CDR-H2 and CDR-H3 sequences of PALF20 as set forth in Tables 15F-1 and 15F-2. In some embodiments, a BCMA ABM comprises CDR-L1, CDR-L2, CDR-L3, CDR-H1, CDR-H2 and CDR-H3 sequences of PALF20 as set forth in Tables 15G-1 and 15G-2. In some embodiments, a BCMA ABM comprises CDR-L1, CDR-L2, CDR-L3, CDR-H1, CDR-H2 and CDR-H3 sequences of PALF20 as set forth in Tables 15H-1 and 15H-2.

In some embodiments, a BCMA ABM comprises CDR-L1, CDR-L2, CDR-L3, CDR-H1, CDR-H2 and CDR-H3 sequences of AB3 as set forth in Tables 15-1 and 15I-2. In some embodiments, a BCMA ABM comprises CDR-L1, CDR-L2, CDR-L3, CDR-H1, CDR-H2 and CDR-H3 sequences of AB3 as set forth in Tables 15J-1 and 15J-2. In some embodiments, a BCMA ABM comprises CDR-L1, CDR-L2, CDR-L3, CDR-H1, CDR-H2 and CDR-H3 sequences of AB3 as set forth in Tables 15K-1 and 15K-2. In some embodiments, a BCMA ABM comprises CDR-L1, CDR-L2, CDR-L3, CDR-H1, CDR-H2 and CDR-H3 sequences of AB3 as set forth in Tables 15L-1 and 15L-2. In some embodiments, a BCMA ABM comprises CDR-L1, CDR-L2, CDR-L3, CDR-H1, CDR-H2 and CDR-H3 sequences of AB3 as set forth in Tables 15M-1 and 15M-2. In some embodiments, a BCMA ABM comprises CDR-L1, CDR-L2, CDR-L3, CDR-H1, CDR-H2 and CDR-H3 sequences of AB3 as set forth in Tables 15N-1 and 15N-2.

In some embodiments, a BCMA ABM comprises CDR-L1, CDR-L2, CDR-L3, CDR-H1, CDR-H2 and CDR-H3 sequences of P1-61 as set forth in Tables 15-1 and 15I-2. In some embodiments, a BCMA ABM comprises CDR-L1, CDR-L2, CDR-L3, CDR-H1, CDR-H2 and CDR-H3 sequences of P1-61 as set forth in Tables 15J-1 and 15J-2. In some embodiments, a BCMA ABM comprises CDR-L1, CDR-L2, CDR-L3, CDR-H1, CDR-H2 and CDR-H3 sequences of P1-61 as set forth in Tables 15K-1 and 15K-2. In some embodiments, a BCMA ABM comprises CDR-L1, CDR-L2, CDR-L3, CDR-H1, CDR-H2 and CDR-H3 sequences of P1-61 as set forth in Tables 15L-1 and 15L-2. In some embodiments, a BCMA ABM comprises CDR-L1, CDR-L2, CDR-L3, CDR-H1, CDR-H2 and CDR-H3 sequences of P1-61 as set forth in Tables 15M-1 and 15M-2. In some embodiments, a BCMA ABM comprises CDR-L1, CDR-L2, CDR-L3, CDR-H1, CDR-H2 and CDR-H3 sequences of P1-61 as set forth in Tables 15N-1 and 15N-2.

In some embodiments, a BCMA ABM comprises CDR-L1, CDR-L2, CDR-L3, CDR-H1, CDR-H2 and CDR-H3 sequences of H2/L2-22 as set forth in Tables 15-1 and 15I-2. In some embodiments, a BCMA ABM comprises CDR-L1, CDR-L2, CDR-L3, CDR-H1, CDR-H2 and CDR-H3 sequences of H2/L2-22 as set forth in Tables 15J-1 and 15J-2. In some embodiments, a BCMA ABM comprises CDR-L1, CDR-L2, CDR-L3, CDR-H1, CDR-H2 and CDR-H3 sequences of H2/L2-22 as set forth in Tables 15K-1 and 15K-2. In some embodiments, a BCMA ABM comprises CDR-L1, CDR-L2, CDR-L3, CDR-H1, CDR-H2 and CDR-H3 sequences of H2/L2-22 as set forth in Tables 15L-1 and 15L-2. In some embodiments, a BCMA ABM comprises CDR-L1, CDR-L2, CDR-L3, CDR-H1, CDR-H2 and CDR-H3 sequences of H2/L2-22 as set forth in Tables 15M-1 and 15M-2. In some embodiments, a BCMA ABM comprises CDR-L1, CDR-L2, CDR-L3, CDR-H1, CDR-H2 and CDR-H3 sequences of H2/L2-22 as set forth in Tables 15N-1 and 15N-2.

In some embodiments, a BCMA ABM comprises CDR-L1, CDR-L2, CDR-L3, CDR-H1, CDR-H2 and CDR-H3 sequences of H2/L2-88 as set forth in Tables 15-1 and 15I-2. In some embodiments, a BCMA ABM comprises CDR-L1, CDR-L2, CDR-L3, CDR-H1, CDR-H2 and CDR-H3 sequences of H2/L2-88 as set forth in Tables 15J-1 and 15J-2. In some embodiments, a BCMA ABM comprises CDR-L1, CDR-L2, CDR-L3, CDR-H1, CDR-H2 and CDR-H3 sequences of H2/L2-88 as set forth in Tables 15K-1 and 15K-2. In some embodiments, a BCMA ABM comprises CDR-L1, CDR-L2, CDR-L3, CDR-H1, CDR-H2 and CDR-H3 sequences of H2/L2-88 as set forth in Tables 15L-1 and 15L-2. In some embodiments, a BCMA ABM comprises CDR-L1, CDR-L2, CDR-L3, CDR-H1, CDR-H2 and CDR-H3 sequences of H2/L2-88 as set forth in Tables 15M-1 and 15M-2. In some embodiments, a BCMA ABM comprises CDR-L1, CDR-L2, CDR-L3, CDR-H1, CDR-H2 and CDR-H3 sequences of H2/L2-88 as set forth in Tables 15N-1 and 15N-2.

In some embodiments, a BCMA ABM comprises CDR-L1, CDR-L2, CDR-L3, CDR-H1, CDR-H2 and CDR-H3 sequences of H2/L2-36 as set forth in Tables 15-1 and 15I-2. In some embodiments, a BCMA ABM comprises CDR-L1, CDR-L2, CDR-L3, CDR-H1, CDR-H2 and CDR-H3 sequences of H2/L2-36 as set forth in Tables 15J-1 and 15J-2. In some embodiments, a BCMA ABM comprises CDR-L1, CDR-L2, CDR-L3, CDR-H1, CDR-H2 and CDR-H3 sequences of H2/L2-36 as set forth in Tables 15K-1 and 15K-2. In some embodiments, a BCMA ABM comprises CDR-L1, CDR-L2, CDR-L3, CDR-H1, CDR-H2 and CDR-H3 sequences of H2/L2-36 as set forth in Tables 15L-1 and 15L-2. In some embodiments, a BCMA ABM comprises CDR-L1, CDR-L2, CDR-L3, CDR-H1, CDR-H2 and CDR-H3 sequences of H2/L2-36 as set forth in Tables 15M-1 and 15M-2. In some embodiments, a BCMA ABM comprises CDR-L1, CDR-L2, CDR-L3, CDR-H1, CDR-H2 and CDR-H3 sequences of H2/L2-36 as set forth in Tables 15N-1 and 15N-2.

In some embodiments, a BCMA ABM comprises CDR-L1, CDR-L2, CDR-L3, CDR-H1, CDR-H2 and CDR-H3 sequences of H2/L2-34 as set forth in Tables 15-1 and 15I-2. In some embodiments, a BCMA ABM comprises CDR-L1, CDR-L2, CDR-L3, CDR-H1, CDR-H2 and CDR-H3 sequences of H2/L2-34 as set forth in Tables 15J-1 and 15J-2. In some embodiments, a BCMA ABM comprises CDR-L1, CDR-L2, CDR-L3, CDR-H1, CDR-H2 and CDR-H3 sequences of H2/L2-34 as set forth in Tables 15K-1 and 15K-2. In some embodiments, a BCMA ABM comprises CDR-L1, CDR-L2, CDR-L3, CDR-H1, CDR-H2 and CDR-H3 sequences of H2/L2-34 as set forth in Tables 15L-1 and 15L-2. In some embodiments, a BCMA ABM comprises CDR-L1, CDR-L2, CDR-L3, CDR-H1, CDR-H2 and CDR-H3 sequences of H2/L2-34 as set forth in Tables 15M-1 and 15M-2. In some embodiments, a BCMA ABM comprises CDR-L1, CDR-L2, CDR-L3, CDR-H1, CDR-H2 and CDR-H3 sequences of H2/L2-34 as set forth in Tables 15N-1 and 15N-2.

In some embodiments, a BCMA ABM comprises CDR-L1, CDR-L2, CDR-L3, CDR-H1, CDR-H2 and CDR-H3 sequences of H2/L2-68 as set forth in Tables 15-1 and 15I-2. In some embodiments, a BCMA ABM comprises CDR-L1, CDR-L2, CDR-L3, CDR-H1, CDR-H2 and CDR-H3 sequences of H2/L2-68 as set forth in Tables 15J-1 and 15J-2. In some embodiments, a BCMA ABM comprises CDR-L1, CDR-L2, CDR-L3, CDR-H1, CDR-H2 and CDR-H3 sequences of H2/L2-68 as set forth in Tables 15K-1 and 15K-2. In some embodiments, a BCMA ABM comprises CDR-L1, CDR-L2, CDR-L3, CDR-H1, CDR-H2 and CDR-H3 sequences of H2/L2-68 as set forth in Tables 15L-1 and 15L-2. In some embodiments, a BCMA ABM comprises CDR-L1, CDR-L2, CDR-L3, CDR-H1, CDR-H2 and CDR-H3 sequences of H2/L2-68 as set forth in Tables 15M-1 and 15M-2. In some embodiments, a BCMA ABM comprises CDR-L1, CDR-L2, CDR-L3, CDR-H1, CDR-H2 and CDR-H3 sequences of H2/L2-68 as set forth in Tables 15N-1 and 15N-2.

In some embodiments, a BCMA ABM comprises CDR-L1, CDR-L2, CDR-L3, CDR-H1, CDR-H2 and CDR-H3 sequences of H2/L2-18 as set forth in Tables 15-1 and 15I-2. In some embodiments, a BCMA ABM comprises CDR-L1, CDR-L2, CDR-L3, CDR-H1, CDR-H2 and CDR-H3 sequences of H2/L2-18 as set forth in Tables 15J-1 and 15J-2. In some embodiments, a BCMA ABM comprises CDR-L1, CDR-L2, CDR-L3, CDR-H1, CDR-H2 and CDR-H3 sequences of H2/L2-18 as set forth in Tables 15K-1 and 15K-2. In some embodiments, a BCMA ABM comprises CDR-L1, CDR-L2, CDR-L3, CDR-H1, CDR-H2 and CDR-H3 sequences of H2/L2-18 as set forth in Tables 15L-1 and 15L-2. In some embodiments, a BCMA ABM comprises CDR-L1, CDR-L2, CDR-L3, CDR-H1, CDR-H2 and CDR-H3 sequences of H2/L2-18 as set forth in Tables 15M-1 and 15M-2. In some embodiments, a BCMA ABM comprises CDR-L1, CDR-L2, CDR-L3, CDR-H1, CDR-H2 and CDR-H3 sequences of H2/L2-18 as set forth in Tables 15N-1 and 15N-2.

In some embodiments, a BCMA ABM comprises CDR-L1, CDR-L2, CDR-L3, CDR-H1, CDR-H2 and CDR-H3 sequences of H2/L2-47 as set forth in Tables 15-1 and 15I-2. In some embodiments, a BCMA ABM comprises CDR-L1, CDR-L2, CDR-L3, CDR-H1, CDR-H2 and CDR-H3 sequences of H2/L2-47 as set forth in Tables 15J-1 and 15J-2. In some embodiments, a BCMA ABM comprises CDR-L1, CDR-L2, CDR-L3, CDR-H1, CDR-H2 and CDR-H3 sequences of H2/L2-47 as set forth in Tables 15K-1 and 15K-2. In some embodiments, a BCMA ABM comprises CDR-L1, CDR-L2, CDR-L3, CDR-H1, CDR-H2 and CDR-H3 sequences of H2/L2-47 as set forth in Tables 15L-1 and 15L-2. In some embodiments, a BCMA ABM comprises CDR-L1, CDR-L2, CDR-L3, CDR-H1, CDR-H2 and CDR-H3 sequences of H2/L2-47 as set forth in Tables 15M-1 and 15M-2. In some embodiments, a BCMA ABM comprises CDR-L1, CDR-L2, CDR-L3, CDR-H1, CDR-H2 and CDR-H3 sequences of H2/L2-47 as set forth in Tables 15N-1 and 15N-2.

In some embodiments, a BCMA ABM comprises CDR-L1, CDR-L2, CDR-L3, CDR-H1, CDR-H2 and CDR-H3 sequences of H2/L2-20 as set forth in Tables 15-1 and 15I-2. In some embodiments, a BCMA ABM comprises CDR-L1, CDR-L2, CDR-L3, CDR-H1, CDR-H2 and CDR-H3 sequences of H2/L2-20 as set forth in Tables 15J-1 and 15J-2. In some embodiments, a BCMA ABM comprises CDR-L1, CDR-L2, CDR-L3, CDR-H1, CDR-H2 and CDR-H3 sequences of H2/L2-20 as set forth in Tables 15K-1 and 15K-2. In some embodiments, a BCMA ABM comprises CDR-L1, CDR-L2, CDR-L3, CDR-H1, CDR-H2 and CDR-H3 sequences of H2/L2-20 as set forth in Tables 15L-1 and 15L-2. In some embodiments, a BCMA ABM comprises CDR-L1, CDR-L2, CDR-L3, CDR-H1, CDR-H2 and CDR-H3 sequences of H2/L2-20 as set forth in Tables 15M-1 and 15M-2. In some embodiments, a BCMA ABM comprises CDR-L1, CDR-L2, CDR-L3, CDR-H1, CDR-H2 and CDR-H3 sequences of H2/L2-20 as set forth in Tables 15N-1 and 15N-2.

In some embodiments, a BCMA ABM comprises CDR-L1, CDR-L2, CDR-L3, CDR-H1, CDR-H2 and CDR-H3 sequences of H2/L2-80 as set forth in Tables 15-1 and 15I-2. In some embodiments, a BCMA ABM comprises CDR-L1, CDR-L2, CDR-L3, CDR-H1, CDR-H2 and CDR-H3 sequences of H2/L2-80 as set forth in Tables 15J-1 and 15J-2. In some embodiments, a BCMA ABM comprises CDR-L1, CDR-L2, CDR-L3, CDR-H1, CDR-H2 and CDR-H3 sequences of H2/L2-80 as set forth in Tables 15K-1 and 15K-2. In some embodiments, a BCMA ABM comprises CDR-L1, CDR-L2, CDR-L3, CDR-H1, CDR-H2 and CDR-H3 sequences of H2/L2-80 as set forth in Tables 15L-1 and 15L-2. In some embodiments, a BCMA ABM comprises CDR-L1, CDR-L2, CDR-L3, CDR-H1, CDR-H2 and CDR-H3 sequences of H2/L2-80 as set forth in Tables 15M-1 and 15M-2. In some embodiments, a BCMA ABM comprises CDR-L1, CDR-L2, CDR-L3, CDR-H1, CDR-H2 and CDR-H3 sequences of H2/L2-80 as set forth in Tables 15N-1 and 15N-2.

In some embodiments, a BCMA ABM comprises CDR-L1, CDR-L2, CDR-L3, CDR-H1, CDR-H2 and CDR-H3 sequences of H2/L2-83 as set forth in Tables 15-1 and 15I-2. In some embodiments, a BCMA ABM comprises CDR-L1, CDR-L2, CDR-L3, CDR-H1, CDR-H2 and CDR-H3 sequences of H2/L2-83 as set forth in Tables 15J-1 and 15J-2. In some embodiments, a BCMA ABM comprises CDR-L1, CDR-L2, CDR-L3, CDR-H1, CDR-H2 and CDR-H3 sequences of H2/L2-83 as set forth in Tables 15K-1 and 15K-2. In some embodiments, a BCMA ABM comprises CDR-L1, CDR-L2, CDR-L3, CDR-H1, CDR-H2 and CDR-H3 sequences of H2/L2-83 as set forth in Tables 15L-1 and 15L-2. In some embodiments, a BCMA ABM comprises CDR-L1, CDR-L2, CDR-L3, CDR-H1, CDR-H2 and CDR-H3 sequences of H2/L2-83 as set forth in Tables 15M-1 and 15M-2. In some embodiments, a BCMA ABM comprises CDR-L1, CDR-L2, CDR-L3, CDR-H1, CDR-H2 and CDR-H3 sequences of H2/L2-83 as set forth in Tables 15N-1 and 15N-2.

In some embodiments, a BCMA ABM comprises CDR-L1, CDR-L2, CDR-L3, CDR-H1, CDR-H2 and CDR-H3 sequences of H3-1 as set forth in Tables 15-1 and 15I-2. In some embodiments, a BCMA ABM comprises CDR-L1, CDR-L2, CDR-L3, CDR-H1, CDR-H2 and CDR-H3 sequences of H3-1 as set forth in Tables 15J-1 and 15J-2. In some embodiments, a BCMA ABM comprises CDR-L1, CDR-L2, CDR-L3, CDR-H1, CDR-H2 and CDR-H3 sequences of H3-1 as set forth in Tables 15K-1 and 15K-2. In some embodiments, a BCMA ABM comprises CDR-L1, CDR-L2, CDR-L3, CDR-H1, CDR-H2 and CDR-H3 sequences of H3-1 as set forth in Tables 15L-1 and 15L-2. In some embodiments, a BCMA ABM comprises CDR-L1, CDR-L2, CDR-L3, CDR-H1, CDR-H2 and CDR-H3 sequences of H3-1 as set forth in Tables 15M-1 and 15M-2. In some embodiments, a BCMA ABM comprises CDR-L1, CDR-L2, CDR-L3, CDR-H1, CDR-H2 and CDR-H3 sequences of H3-1 as set forth in Tables 15N-1 and 15N-2.

In some embodiments, a BCMA ABM comprises CDR-L1, CDR-L2, CDR-L3, CDR-H1, CDR-H2 and CDR-H3 sequences of H3-2 as set forth in Tables 15-1 and 15I-2. In some embodiments, a BCMA ABM comprises CDR-L1, CDR-L2, CDR-L3, CDR-H1, CDR-H2 and CDR-H3 sequences of H3-2 as set forth in Tables 15J-1 and 15J-2. In some embodiments, a BCMA ABM comprises CDR-L1, CDR-L2, CDR-L3, CDR-H1, CDR-H2 and CDR-H3 sequences of H3-2 as set forth in Tables 15K-1 and 15K-2. In some embodiments, a BCMA ABM comprises CDR-L1, CDR-L2, CDR-L3, CDR-H1, CDR-H2 and CDR-H3 sequences of H3-2 as set forth in Tables 15L-1 and 15L-2. In some embodiments, a BCMA ABM comprises CDR-L1, CDR-L2, CDR-L3, CDR-H1, CDR-H2 and CDR-H3 sequences of H3-2 as set forth in Tables 15M-1 and 15M-2. In some embodiments, a BCMA ABM comprises CDR-L1, CDR-L2, CDR-L3, CDR-H1, CDR-H2 and CDR-H3 sequences of H3-2 as set forth in Tables 15N-1 and 15N-2.

In some embodiments, a BCMA ABM comprises CDR-L1, CDR-L2, CDR-L3, CDR-H1, CDR-H2 and CDR-H3 sequences of H3-3 as set forth in Tables 15-1 and 15I-2. In some embodiments, a BCMA ABM comprises CDR-L1, CDR-L2, CDR-L3, CDR-H1, CDR-H2 and CDR-H3 sequences of H3-3 as set forth in Tables 15J-1 and 15J-2. In some embodiments, a BCMA ABM comprises CDR-L1, CDR-L2, CDR-L3, CDR-H1, CDR-H2 and CDR-H3 sequences of H3-3 as set forth in Tables 15K-1 and 15K-2. In some embodiments, a BCMA ABM comprises CDR-L1, CDR-L2, CDR-L3, CDR-H1, CDR-H2 and CDR-H3 sequences of H3-3 as set forth in Tables 15L-1 and 15L-2. In some embodiments, a BCMA ABM comprises CDR-L1, CDR-L2, CDR-L3, CDR-H1, CDR-H2 and CDR-H3 sequences of H3-3 as set forth in Tables 15M-1 and 15M-2. In some embodiments, a BCMA ABM comprises CDR-L1, CDR-L2, CDR-L3, CDR-H1, CDR-H2 and CDR-H3 sequences of H3-3 as set forth in Tables 15N-1 and 15N-2.

In some embodiments, a BCMA ABM comprises CDR-L1, CDR-L2, CDR-L3, CDR-H1, CDR-H2 and CDR-H3 sequences of H3-4 as set forth in Tables 15-1 and 15I-2. In some embodiments, a BCMA ABM comprises CDR-L1, CDR-L2, CDR-L3, CDR-H1, CDR-H2 and CDR-H3 sequences of H3-4 as set forth in Tables 15J-1 and 15J-2. In some embodiments, a BCMA ABM comprises CDR-L1, CDR-L2, CDR-L3, CDR-H1, CDR-H2 and CDR-H3 sequences of H3-4 as set forth in Tables 15K-1 and 15K-2. In some embodiments, a BCMA ABM comprises CDR-L1, CDR-L2, CDR-L3, CDR-H1, CDR-H2 and CDR-H3 sequences of H3-4 as set forth in Tables 15L-1 and 15L-2. In some embodiments, a BCMA ABM comprises CDR-L1, CDR-L2, CDR-L3, CDR-H1, CDR-H2 and CDR-H3 sequences of H3-4 as set forth in Tables 15M-1 and 15M-2. In some embodiments, a BCMA ABM comprises CDR-L1, CDR-L2, CDR-L3, CDR-H1, CDR-H2 and CDR-H3 sequences of H3-4 as set forth in Tables 15N-1 and 15N-2.

In some embodiments, a BCMA ABM comprises CDR-L1, CDR-L2, CDR-L3, CDR-H1, CDR-H2 and CDR-H3 sequences of H3-5 as set forth in Tables 15-1 and 15I-2. In some embodiments, a BCMA ABM comprises CDR-L1, CDR-L2, CDR-L3, CDR-H1, CDR-H2 and CDR-H3 sequences of H3-5 as set forth in Tables 15J-1 and 15J-2. In some embodiments, a BCMA ABM comprises CDR-L1, CDR-L2, CDR-L3, CDR-H1, CDR-H2 and CDR-H3 sequences of H3-5 as set forth in Tables 15K-1 and 15K-2. In some embodiments, a BCMA ABM comprises CDR-L1, CDR-L2, CDR-L3, CDR-H1, CDR-H2 and CDR-H3 sequences of H3-5 as set forth in Tables 15L-1 and 15L-2. In some embodiments, a BCMA ABM comprises CDR-L1, CDR-L2, CDR-L3, CDR-H1, CDR-H2 and CDR-H3 sequences of H3-5 as set forth in Tables 15M-1 and 15M-2. In some embodiments, a BCMA ABM comprises CDR-L1, CDR-L2, CDR-L3, CDR-H1, CDR-H2 and CDR-H3 sequences of H3-5 as set forth in Tables 15N-1 and 15N-2.

In some embodiments, a BCMA ABM comprises CDR-L1, CDR-L2, CDR-L3, CDR-H1, CDR-H2 and CDR-H3 sequences of H3-6 as set forth in Tables 15-1 and 15I-2. In some embodiments, a BCMA ABM comprises CDR-L1, CDR-L2, CDR-L3, CDR-H1, CDR-H2 and CDR-H3 sequences of H3-6 as set forth in Tables 15J-1 and 15J-2. In some embodiments, a BCMA ABM comprises CDR-L1, CDR-L2, CDR-L3, CDR-H1, CDR-H2 and CDR-H3 sequences of H3-6 as set forth in Tables 15K-1 and 15K-2. In some embodiments, a BCMA ABM comprises CDR-L1, CDR-L2, CDR-L3, CDR-H1, CDR-H2 and CDR-H3 sequences of H3-6 as set forth in Tables 15L-1 and 15L-2. In some embodiments, a BCMA ABM comprises CDR-L1, CDR-L2, CDR-L3, CDR-H1, CDR-H2 and CDR-H3 sequences of H3-6 as set forth in Tables 15M-1 and 15M-2. In some embodiments, a BCMA ABM comprises CDR-L1, CDR-L2, CDR-L3, CDR-H1, CDR-H2 and CDR-H3 sequences of H3-6 as set forth in Tables 15N-1 and 15N-2.

In some embodiments, a BCMA ABM comprises CDR-L1, CDR-L2, CDR-L3, CDR-H1, CDR-H2 and CDR-H3 sequences of H3-7 as set forth in Tables 15-1 and 15I-2. In some embodiments, a BCMA ABM comprises CDR-L1, CDR-L2, CDR-L3, CDR-H1, CDR-H2 and CDR-H3 sequences of H3-7 as set forth in Tables 15J-1 and 15J-2. In some embodiments, a BCMA ABM comprises CDR-L1, CDR-L2, CDR-L3, CDR-H1, CDR-H2 and CDR-H3 sequences of H3-7 as set forth in Tables 15K-1 and 15K-2. In some embodiments, a BCMA ABM comprises CDR-L1, CDR-L2, CDR-L3, CDR-H1, CDR-H2 and CDR-H3 sequences of H3-7 as set forth in Tables 15L-1 and 15L-2. In some embodiments, a BCMA ABM comprises CDR-L1, CDR-L2, CDR-L3, CDR-H1, CDR-H2 and CDR-H3 sequences of H3-7 as set forth in Tables 15M-1 and 15M-2. In some embodiments, a BCMA ABM comprises CDR-L1, CDR-L2, CDR-L3, CDR-H1, CDR-H2 and CDR-H3 sequences of H3-7 as set forth in Tables 15N-1 and 15N-2.

In some embodiments, a BCMA ABM comprises CDR-L1, CDR-L2, CDR-L3, CDR-H1, CDR-H2 and CDR-H3 sequences of H3-8 as set forth in Tables 15-1 and 15I-2. In some embodiments, a BCMA ABM comprises CDR-L1, CDR-L2, CDR-L3, CDR-H1, CDR-H2 and CDR-H3 sequences of H3-8 as set forth in Tables 15J-1 and 15J-2. In some embodiments, a BCMA ABM comprises CDR-L1, CDR-L2, CDR-L3, CDR-H1, CDR-H2 and CDR-H3 sequences of H3-8 as set forth in Tables 15K-1 and 15K-2. In some embodiments, a BCMA ABM comprises CDR-L1, CDR-L2, CDR-L3, CDR-H1, CDR-H2 and CDR-H3 sequences of H3-8 as set forth in Tables 15L-1 and 15L-2. In some embodiments, a BCMA ABM comprises CDR-L1, CDR-L2, CDR-L3, CDR-H1, CDR-H2 and CDR-H3 sequences of H3-8 as set forth in Tables 15M-1 and 15M-2. In some embodiments, a BCMA ABM comprises CDR-L1, CDR-L2, CDR-L3, CDR-H1, CDR-H2 and CDR-H3 sequences of H3-8 as set forth in Tables 15N-1 and 15N-2.

In some embodiments, a BCMA ABM comprises CDR-L1, CDR-L2, CDR-L3, CDR-H1, CDR-H2 and CDR-H3 sequences of H3-9 as set forth in Tables 15-1 and 15I-2. In some embodiments, a BCMA ABM comprises CDR-L1, CDR-L2, CDR-L3, CDR-H1, CDR-H2 and CDR-H3 sequences of H3-9 as set forth in Tables 15J-1 and 15J-2. In some embodiments, a BCMA ABM comprises CDR-L1, CDR-L2, CDR-L3, CDR-H1, CDR-H2 and CDR-H3 sequences of H3-9 as set forth in Tables 15K-1 and 15K-2. In some embodiments, a BCMA ABM comprises CDR-L1, CDR-L2, CDR-L3, CDR-H1, CDR-H2 and CDR-H3 sequences of H3-9 as set forth in Tables 15L-1 and 15L-2. In some embodiments, a BCMA ABM comprises CDR-L1, CDR-L2, CDR-L3, CDR-H1, CDR-H2 and CDR-H3 sequences of H3-9 as set forth in Tables 15M-1 and 15M-2. In some embodiments, a BCMA ABM comprises CDR-L1, CDR-L2, CDR-L3, CDR-H1, CDR-H2 and CDR-H3 sequences of H3-9 as set forth in Tables 15N-1 and 15N-2.

In some embodiments, a BCMA ABM comprises CDR-L1, CDR-L2, CDR-L3, CDR-H1, CDR-H2 and CDR-H3 sequences of H3-10 as set forth in Tables 15-1 and 15I-2. In some embodiments, a BCMA ABM comprises CDR-L1, CDR-L2, CDR-L3, CDR-H1, CDR-H2 and CDR-H3 sequences of H3-10 as set forth in Tables 15J-1 and 15J-2. In some embodiments, a BCMA ABM comprises CDR-L1, CDR-L2, CDR-L3, CDR-H1, CDR-H2 and CDR-H3 sequences of H3-10 as set forth in Tables 15K-1 and 15K-2. In some embodiments, a BCMA ABM comprises CDR-L1, CDR-L2, CDR-L3, CDR-H1, CDR-H2 and CDR-H3 sequences of H3-10 as set forth in Tables 15L-1 and 15L-2. In some embodiments, a BCMA ABM comprises CDR-L1, CDR-L2, CDR-L3, CDR-H1, CDR-H2 and CDR-H3 sequences of H3-10 as set forth in Tables 15M-1 and 15M-2. In some embodiments, a BCMA ABM comprises CDR-L1, CDR-L2, CDR-L3, CDR-H1, CDR-H2 and CDR-H3 sequences of H3-10 as set forth in Tables 15N-1 and 15N-2.

In some embodiments, a BCMA ABM comprises CDR-L1, CDR-L2, CDR-L3, CDR-H1, CDR-H2 and CDR-H3 sequences of H3-11 as set forth in Tables 15-1 and 15I-2. In some embodiments, a BCMA ABM comprises CDR-L1, CDR-L2, CDR-L3, CDR-H1, CDR-H2 and CDR-H3 sequences of H3-11 as set forth in Tables 15J-1 and 15J-2. In some embodiments, a BCMA ABM comprises CDR-L1, CDR-L2, CDR-L3, CDR-H1, CDR-H2 and CDR-H3 sequences of H3-11 as set forth in Tables 15K-1 and 15K-2. In some embodiments, a BCMA ABM comprises CDR-L1, CDR-L2, CDR-L3, CDR-H1, CDR-H2 and CDR-H3 sequences of H3-11 as set forth in Tables 15L-1 and 15L-2. In some embodiments, a BCMA ABM comprises CDR-L1, CDR-L2, CDR-L3, CDR-H1, CDR-H2 and CDR-H3 sequences of H3-11 as set forth in Tables 15M-1 and 15M-2. In some embodiments, a BCMA ABM comprises CDR-L1, CDR-L2, CDR-L3, CDR-H1, CDR-H2 and CDR-H3 sequences of H3-11 as set forth in Tables 15N-1 and 15N-2.

In some embodiments, a BCMA ABM comprises CDR-L1, CDR-L2, CDR-L3, CDR-H1, CDR-H2 and CDR-H3 sequences of H3-12 as set forth in Tables 15-1 and 15I-2. In some embodiments, a BCMA ABM comprises CDR-L1, CDR-L2, CDR-L3, CDR-H1, CDR-H2 and CDR-H3 sequences of H3-12 as set forth in Tables 15J-1 and 15J-2. In some embodiments, a BCMA ABM comprises CDR-L1, CDR-L2, CDR-L3, CDR-H1, CDR-H2 and CDR-H3 sequences of H3-12 as set forth in Tables 15K-1 and 15K-2. In some embodiments, a BCMA ABM comprises CDR-L1, CDR-L2, CDR-L3, CDR-H1, CDR-H2 and CDR-H3 sequences of H3-12 as set forth in Tables 15L-1 and 15L-2. In some embodiments, a BCMA ABM comprises CDR-L1, CDR-L2, CDR-L3, CDR-H1, CDR-H2 and CDR-H3 sequences of H3-12 as set forth in Tables 15M-1 and 15M-2. In some embodiments, a BCMA ABM comprises CDR-L1, CDR-L2, CDR-L3, CDR-H1, CDR-H2 and CDR-H3 sequences of H3-12 as set forth in Tables 15N-1 and 15N-2.

In some embodiments, a BCMA ABM comprises CDR-L1, CDR-L2, CDR-L3, CDR-H1, CDR-H2 and CDR-H3 sequences of H3-13 as set forth in Tables 15-1 and 15I-2. In some embodiments, a BCMA ABM comprises CDR-L1, CDR-L2, CDR-L3, CDR-H1, CDR-H2 and CDR-H3 sequences of H3-13 as set forth in Tables 15J-1 and 15J-2. In some embodiments, a BCMA ABM comprises CDR-L1, CDR-L2, CDR-L3, CDR-H1, CDR-H2 and CDR-H3 sequences of H3-13 as set forth in Tables 15K-1 and 15K-2. In some embodiments, a BCMA ABM comprises CDR-L1, CDR-L2, CDR-L3, CDR-H1, CDR-H2 and CDR-H3 sequences of H3-13 as set forth in Tables 15L-1 and 15L-2. In some embodiments, a BCMA ABM comprises CDR-L1, CDR-L2, CDR-L3, CDR-H1, CDR-H2 and CDR-H3 sequences of H3-13 as set forth in Tables 15M-1 and 15M-2. In some embodiments, a BCMA ABM comprises CDR-L1, CDR-L2, CDR-L3, CDR-H1, CDR-H2 and CDR-H3 sequences of H3-13 as set forth in Tables 15N-1 and 15N-2.

In some embodiments, a BCMA ABM comprises CDR-L1, CDR-L2, CDR-L3, CDR-H1, CDR-H2 and CDR-H3 sequences of H3-14 as set forth in Tables 15-1 and 15I-2. In some embodiments, a BCMA ABM comprises CDR-L1, CDR-L2, CDR-L3, CDR-H1, CDR-H2 and CDR-H3 sequences of H3-14 as set forth in Tables 15J-1 and 15J-2. In some embodiments, a BCMA ABM comprises CDR-L1, CDR-L2, CDR-L3, CDR-H1, CDR-H2 and CDR-H3 sequences of H3-14 as set forth in Tables 15K-1 and 15K-2. In some embodiments, a BCMA ABM comprises CDR-L1, CDR-L2, CDR-L3, CDR-H1, CDR-H2 and CDR-H3 sequences of H3-14 as set forth in Tables 15L-1 and 15L-2. In some embodiments, a BCMA ABM comprises CDR-L1, CDR-L2, CDR-L3, CDR-H1, CDR-H2 and CDR-H3 sequences of H3-14 as set forth in Tables 15M-1 and 15M-2. In some embodiments, a BCMA ABM comprises CDR-L1, CDR-L2, CDR-L3, CDR-H1, CDR-H2 and CDR-H3 sequences of H3-14 as set forth in Tables 15N-1 and 15N-2.

In some embodiments, a BCMA ABM comprises CDR-L1, CDR-L2, CDR-L3, CDR-H1, CDR-H2 and CDR-H3 sequences of H3-15 as set forth in Tables 15-1 and 15I-2. In some embodiments, a BCMA ABM comprises CDR-L1, CDR-L2, CDR-L3, CDR-H1, CDR-H2 and CDR-H3 sequences of H3-15 as set forth in Tables 15J-1 and 15J-2. In some embodiments, a BCMA ABM comprises CDR-L1, CDR-L2, CDR-L3, CDR-H1, CDR-H2 and CDR-H3 sequences of H3-15 as set forth in Tables 15K-1 and 15K-2. In some embodiments, a BCMA ABM comprises CDR-L1, CDR-L2, CDR-L3, CDR-H1, CDR-H2 and CDR-H3 sequences of H3-15 as set forth in Tables 15L-1 and 15L-2. In some embodiments, a BCMA ABM comprises CDR-L1, CDR-L2, CDR-L3, CDR-H1, CDR-H2 and CDR-H3 sequences of H3-15 as set forth in Tables 15M-1 and 15M-2. In some embodiments, a BCMA ABM comprises CDR-L1, CDR-L2, CDR-L3, CDR-H1, CDR-H2 and CDR-H3 sequences of H3-15 as set forth in Tables 15N-1 and 15N-2.

In some embodiments, a BCMA ABM comprises a light chain variable sequence and/or heavy chain variable sequence of AB1 as set forth in Table 15O-1 and Table 15O-2. In some embodiments, a BCMA ABM comprises a light chain variable sequence and/or heavy chain variable sequence of AB2 as set forth in Table 15O-1 and Table 15O-2. In some embodiments, a BCMA ABM comprises a light chain variable sequence and/or heavy chain variable sequence of R1F2 as set forth in Table 15O-1 and Table 15O-2. In some embodiments, a BCMA ABM comprises a light chain variable sequence and/or heavy chain variable sequence of PALF03 as set forth in Table 15O-1 and Table 15O-2. In some embodiments, a BCMA ABM comprises a light chain variable sequence and/or heavy chain variable sequence of PALF04 as set forth in Table 15O-1 and Table 15O-2. In some embodiments, a BCMA ABM comprises a light chain variable sequence and/or heavy chain variable sequence of PALF05 as set forth in Table 15O-1 and Table 15O-2. In some embodiments, a BCMA ABM comprises a light chain variable sequence and/or heavy chain variable sequence of PALF06 as set forth in Table 15O-1 and Table 15O-2. In some embodiments, a BCMA ABM comprises a light chain variable sequence and/or heavy chain variable sequence of PALF07 as set forth in Table 15O-1 and Table 15O-2. In some embodiments, a BCMA ABM comprises a light chain variable sequence and/or heavy chain variable sequence of PALF08 as set forth in Table 15O-1 and Table 15O-2. In some embodiments, a BCMA ABM comprises a light chain variable sequence and/or heavy chain variable sequence of PALF09 as set forth in Table 15O-1 and Table 15O-2. In some embodiments, a BCMA ABM comprises a light chain variable sequence and/or heavy chain variable sequence of PALF12 as set forth in Table 15O-1 and Table 15O-2. In some embodiments, a BCMA ABM comprises a light chain variable sequence and/or heavy chain variable sequence of PALF13 as set forth in Table 15O-1 and Table 15O-2. In some embodiments, a BCMA ABM comprises a light chain variable sequence and/or heavy chain variable sequence of PALF14 as set forth in Table 15O-1 and Table 15O-2. In some embodiments, a BCMA ABM comprises a light chain variable sequence and/or heavy chain variable sequence of PALF15 as set forth in Table 15O-1 and Table 15O-2. In some embodiments, a BCMA ABM comprises a light chain variable sequence and/or heavy chain variable sequence of PALF16 as set forth in Table 15O-1 and Table 15O-2. In some embodiments, a BCMA ABM comprises a light chain variable sequence and/or heavy chain variable sequence of PALF17 as set forth in Table 15O-1 and Table 15O-2. In some embodiments, a BCMA ABM comprises a light chain variable sequence and/or heavy chain variable sequence of PALF18 as set forth in Table 15O-1 and Table 15O-2. In some embodiments, a BCMA ABM comprises a light chain variable sequence and/or heavy chain variable sequence of PALF19 as set forth in Table 15O-1 and Table 15O-2. In some embodiments, a BCMA ABM comprises a light chain variable sequence and/or heavy chain variable sequence of PALF20 as set forth in Table 15O-1 and Table 15O-2. In some embodiments, a BCMA ABM comprises a light chain variable sequence and/or heavy chain variable sequence of AB3 as set forth in Table 15O-1 and Table 15O-2. In some embodiments, a BCMA ABM comprises a light chain variable sequence and/or heavy chain variable sequence of PI-61 as set forth in Table 15O-1 and Table 15O-2. In some embodiments, a BCMA ABM comprises a light chain variable sequence and/or heavy chain variable sequence of H3-1 as set forth in Table 15O-1 and Table 15O-2. In some embodiments, a BCMA ABM comprises a light chain variable sequence and/or heavy chain variable sequence of H3-2 as set forth in Table 15O-1 and Table 15O-2. In some embodiments, a BCMA ABM comprises a light chain variable sequence and/or heavy chain variable sequence of H3-3 as set forth in Table 15O-1 and Table 15O-2. In some embodiments, a BCMA ABM comprises a light chain variable sequence and/or heavy chain variable sequence of H3-4 as set forth in Table 15O-1 and Table 15O-2. In some embodiments, a BCMA ABM comprises a light chain variable sequence and/or heavy chain variable sequence of H3-5 as set forth in Table 15O-1 and Table 15O-2. In some embodiments, a BCMA ABM comprises a light chain variable sequence and/or heavy chain variable sequence of H3-6 as set forth in Table 15O-1 and Table 15O-2. In some embodiments, a BCMA ABM comprises a light chain variable sequence and/or heavy chain variable sequence of H3-7 as set forth in Table 15O-1 and Table 15O-2. In some embodiments, a BCMA ABM comprises a light chain variable sequence and/or heavy chain variable sequence of H3-8 as set forth in Table 15O-1 and Table 15O-2. In some embodiments, a BCMA ABM comprises a light chain variable sequence and/or heavy chain variable sequence of H3-9 as set forth in Table 15O-1 and Table 15O-2. In some embodiments, a BCMA ABM comprises a light chain variable sequence and/or heavy chain variable sequence of H3-10 as set forth in Table 15O-1 and Table 15O-2. In some embodiments, a BCMA ABM comprises a light chain variable sequence and/or heavy chain variable sequence of H3-11 as set forth in Table 15O-1 and Table 15O-2. In some embodiments, a BCMA ABM comprises a light chain variable sequence and/or heavy chain variable sequence of H3-12 as set forth in Table 15O-1 and Table 15O-2. In some embodiments, a BCMA ABM comprises a light chain variable sequence and/or heavy chain variable sequence of H3-13 as set forth in Table 15O-1 and Table 15O-2. In some embodiments, a BCMA ABM comprises a light chain variable sequence and/or heavy chain variable sequence of H3-14 as set forth in Table 15O-1 and Table 15O-2. In some embodiments, a BCMA ABM comprises a light chain variable sequence and/or heavy chain variable sequence of H3-15 as set forth in Table 15O-1 and Table 15O-2.

In some embodiments, a BCMA ABM comprises a scFv sequence of H2/L2-88 as set forth in Table 15P. In some embodiments, a BCMA ABM comprises a scFv sequence of H2/L2-36 as set forth in Table 15P. In some embodiments, a BCMA ABM comprises a scFv sequence of H2/L2-34 as set forth in Table 15P. In some embodiments, a BCMA ABM comprises a scFv sequence of H2/L2-68 as set forth in Table 15P. In some embodiments, a BCMA ABM comprises a scFv sequence of H2/L2-18 as set forth in Table 15P. In some embodiments, a BCMA ABM comprises a scFv sequence of H2/L2-47 as set forth in Table 15P. In some embodiments, a BCMA ABM comprises a scFv sequence of H2/L2-20 as set forth in Table 15P. In some embodiments, a BCMA ABM comprises a scFv sequence of H2/L2-80 as set forth in Table 15P. In some embodiments, a BCMA ABM comprises a scFv sequence of H2/L2-83 as set forth in Table 15P.

Given that each BCMA ABM binds BCMA, and that antigen binding specificity is provided primarily by the CDR-H1, CDR-H2, CDR-H3, CDR-L1, CDR-L2, and CDR-L3 regions, the CDR-H1, CDR-H2, CDR-H3, CDR-L1, CDR-L2, and CDR-L3 sequences can be “mixed and matched”. Such “mixed and matched” BCMA ABMs can be tested using known binding assays and those described in the Examples (e.g., ELISAs). When VH CDR sequences are mixed and matched, the CDR-H1, CDR-H2 and/or CDR-H3 sequence from a particular VH sequence should be replaced with a structurally similar CDR sequence(s). Likewise, when VL CDR sequences are mixed and matched, the CDR-L1, CDR-L2 and/or CDR-L3 sequence from a particular VL sequence should be replaced with a structurally similar CDR sequence(s). It will be readily apparent to the ordinarily skilled artisan that novel VH and VL sequences can be created by substituting one or more VH and/or VL CDR region sequences with structurally similar sequences from CDR sequences shown herein for monoclonal antibodies or other BCMA ABMs of the present disclosure.

In some embodiments, a BCMA ABM comprises a VL sequence selected from the VL sequences set forth in Table 15O-1 and a VH sequence selected the VH sequences set forth in Table 15O-2. In some embodiments, a BCMA ABM comprises a CDR-H1 sequence selected from the CDR-H1 sequences set forth in Table 15A-2, Table 15B-2, Table 15C-2, Table 15D-2, Table 15E-2, Table 15F-2, Table 15G-2, Table 15H-2, Table 15I-2, Table 15J-2, Table 15K-2, Table 15L-2, Table 15M-2, and Table 15N-2; a CDR-H2 sequence selected from the CDR-H2 sequences set forth in Table 15A-2, Table 15B-2, Table 15C-2, Table 15D-2, Table 15E-2, Table 15F-2, Table 15G-2, Table 15H-2, Table 15I-2, Table 15J-2, Table 15K-2, Table 15L-2, Table 15M-2, and Table 15N-2; a CDR-H3 sequence selected from the CDR-H3 sequences set forth in Table 15A-2, Table 15B-2, Table 15C-2, Table 15D-2, Table 15E-2, Table 15F-2, Table 15G-2, Table 15H-2, Table 15I-2, Table 15J-2, Table 15K-2, Table 15L-2, Table 15M-2, and Table 15N-2; a CDR-L1 sequence selected from the CDR-L1 sequences set forth in Table 15A-1, Table 15B-1, Table 15C-1, Table 15D-1, Table 15E-1, Table 15F-1, Table 15G-1, Table 15H-1, Table 15I-1, Table 15J-1, Table 15K-1(a), Table 15K-1(b), Table 15L-1, Table 15M-1, Table 15N-1(a), and Table 15N-1(b); a CDR-L2 sequence selected from the CDR-L2 sequences set forth in Table 15A-1, Table 15B-1, Table 15C-1, Table 15D-1, Table 15E-1, Table 15F-1, Table 15G-1, Table 15H-1, Table 15I-1, Table 15J-1, Table 15K-1(a), Table 15K-1(b), Table 15L-1, Table 15M-1, Table 15N-1(a), and Table 15N-1(b); and a CDR-L3 sequence selected from the CDR-L3 sequences set forth in Table 15A-1, Table 15B-1, Table 15C-1, Table 15D-1, Table 15E-1, Table 15F-1, Table 15G-1, Table 15H-1, Table 15I-1, Table 15J-1, Table 15K-1(a), Table 15K-1(b), Table 15L-1, Table 15M-1, Table 15N-1(a), and Table 15N-1(b).

### 7.7.2. CD19

In certain aspects, the present disclosure provides MBMs and combinations of MBMs in which ABM2 and/or ABM5 binds specifically to CD19. CD19 is found on mature B cells but not on plasma cells. CD19 is expressed during early pre-B cell development and remains until plasma cell differentiation. CD19 is expressed on both normal B cells and malignant B cells whose abnormal growth can lead to B-cell lymphomas. For example, CD19 is expressed on B-cell lineage malignancies, including, but not limited to non-Hodgkin's lymphoma (B-NHL), chronic lymphocytic leukemia, and acute lymphoblastic leukemia.

Exemplary CDR and variable domain sequences that can be incorporated into an ABM that specifically binds to CD19 are set forth in Table 16 below.

TABLE 16
CD19 Binders

| | | | SEQ ID |
|---|---|---|---|
| Name | Domain | Sequence | NO: |
| CD19-H1 | CDR-H1 | DYGVS | 1341 |
| CD19-H2A | CDR-H2 | VIWGSETTYYNSALKS | 1342 |
| CD19-H2B | CDR-H2 | VIWGSETTYYSSSLKS | 1343 |
| CD19-H2C | CDR-H2 | VIWGSETTYYQSSLKS | 1344 |
| CD19-H2D | CDR-H2 | VIWGSETTYYNSSLKS | 1345 |
| CD19-H3 | CDR-H3 | HYYYGGSYAMDY | 1346 |
| CD19-L1 | CDR-L1 | RASQDISKYLN | 1347 |
| CD19-L2 | CDR-L2 | HTSRLHS | 1348 |
| CD19-L3 | CDR-L3 | QQGNTLPYT | 1349 |
| CD19-VHA | VH | EVKLQESGPGLVAPSQSLSVTCTVSGVSLPDYGVSWI | 1350 |
| | | RQPPRKGLEWLGVIWGSETTYYNSALKSRLTIIKDNS | |
| | | KSQVFLKMNSLQTDDTAIYYCAKHYYYGGSYAMDYWG | |
| | | QGTSVTVSS | |
| CD19-VHB | VH | QVQLQESGPGLVKPSETLSLTCTVSGVSLPDYGVSWI | 1351 |
| | | RQPPGKGLEWIGVIWGSETTYYSSSLKSRVTISKDNS | |
| | | KNQVSLKLSSVTAADTAVYYCAKHYYYGGSYAMDYWG | |
| | | QGTLVTVSS | |
| CD19-VHC | VH | QVQLQESGPGLVKPSETLSLTCTVSGVSLPDYGVSWI | 1352 |
| | | RQPPGKGLEWIGVIWGSETTYYQSSLKSRVTISKDNS | |
| | | KNQVSLKLSSVTAADTAVYYCAKHYYYGGSYAMDYWG | |
| | | QGTLVTVSS | |
| CD19-VHD | VH | QVQLQESGPGLVKPSETLSLTCTVSGVSLPDYGVSWI | 1353 |
| | | RQPPGKGLEWIGVIWGSETTYYNSSLKSRVTISKDNS | |
| | | KNQVSLKLSSVTAADTAVYYCAKHYYYGGSYAMDYWG | |
| | | QGTLVTVSS | |
| CD19-VHE | VH | QVQLLESGAELVRPGSSVKISCKASGYAFSSYWMNWV | 1354 |
| | | KQRPGQGLEWIGQIYPGDGDTNYNGKFKGQATLTADK | |
| | | SSSTAYMQLSGLTSEDSAVYSCARKTISSVVDFYFDY | |
| | | WGQGTTVT | |
| CD19-VLA | VL | DIQMTQTTSSLSASLGDRVTISCRASQDISKYLNWYQ | 1355 |
| | | QKPDGTVKLLIYHTSRLHSGVPSRFSGSGSGTDYSLT | |
| | | ISNLEQEDIATYFCQQGNTLPYTFGGGTKLEIT | |
| CD19-VLB | VL | EIVMTQSPATLSLSPGERATLSCRASQDISKYLNWYQ | 1356 |
| | | QKPGQAPRLLIYHTSRLHSGIPARFSGSGSGTDYTLT | |
| | | ISSLQPEDFAVYFCQQGNTLPYTFGQGTKLEIK | |
| CD19-VLE | VL | ELVLTQSPKFMSTSVGDRVSVTCKASQNVGTNVAWYQ | 1357 |
| | | QKPGQSPKPLIYSATYRNSGVPDRFTGSGSGTDFTLT | |
| | | ITNVQSKDLADYFYFCQYNRYPYTSGGGTKLEIKRRS | |
| CD19-scFv1 | scFv | EIVMTQSPATLSLSPGERATLSCRASQDISKYLNWYQ | 1358 |
| | | QKPGQAPRLLIYHTSRLHSGIPARFSGSGSGTDYTLT | |
| | | ISSLQPEDFAVYFCQQGNTLPYTFGQGTKLEIKGGGG | |
| | | SGGGGSGGGGSQVQLQESGPGLVKPSETLSLTCTVSG | |
| | | VSLPDYGVSWIRQPPGKGLEWIGVIWGSETTYYSSSL | |
| | | KSRVTISKDNSKNQVSLKLSSVTAADTAVYYCAKHYY | |
| | | YGGSYAMDYWGQGTLVTVSS | |
| CD19-scFv2 | scFv | EIVMTQSPATLSLSPGERATLSCRASQDISKYLNWYQ | 1359 |
| | | QKPGQAPRLLIYHTSRLHSGIPARFSGSGSGTDYTLT | |
| | | ISSLQPEDFAVYFCQQGNTLPYTFGQGTKLEIKGGGG | |
| | | SGGGGSGGGGSQVQLQESGPGLVKPSETLSLTCTVSG | |
| | | VSLPDYGVSWIRQPPGKGLEWIGVIWGSETTYYQSSL | |
| | | KSRVTISKDNSKNQVSLKLSSVTAADTAVYYCAKHYY | |
| | | YGGSYAMDYWGQGTLVTVSS | |
| CD19-scFv3 | scFv | QVQLQESGPGLVKPSETLSLTCTVSGVSLPDYGVSWI | 1360 |
| | | RQPPGKGLEWIGVIWGSETTYYSSSLKSRVTISKDNS | |
| | | KNQVSLKLSSVTAADTAVYYCAKHYYYGGSYAMDYWG | |
| | | QGTLVTVSSGGGGSGGGGSGGGGSEIVMTQSPATLSL | |
| | | SPGERATLSCRASQDISKYLNWYQQKPGQAPRLLIYH | |
| | | TSRLHSGIPARFSGSGSGTDYTLTISSLQPEDFAVYF | |
| | | CQQGNTLPYTFGQGTKLEIK | |
| CD19-scFv4 | scFv | QVQLQESGPGLVKPSETLSLTCTVSGVSLPDYGVSWI | 1361 |
| | | RQPPGKGLEWIGVIWGSETTYYQSSLKSRVTISKDNS | |
| | | KNQVSLKLSSVTAADTAVYYCAKHYYYGGSYAMDYWG | |
| | | QGTLVTVSSGGGGSGGGGSGGGGSEIVMTQSPATLSL | |
| | | SPGERATLSCRASQDISKYLNWYQQKPGQAPRLLIYH | |
| | | TSRLHSGIPARFSGSGSGTDYTLTISSLQPEDFAVYF | |
| | | CQQGNTLPYTFGQGTKLEIK | |
| CD19-scFv5 | scFv | EIVMTQSPATLSLSPGERATLSCRASQDISKYLNWYQ | 1362 |
| | | QKPGQAPRLLIYHTSRLHSGIPARFSGSGSGTDYTLT | |
| | | ISSLQPEDFAVYFCQQGNTLPYTFGQGTKLEIKGGGG | |
| | | SGGGGSGGGGSGGGGSQVQLQESGPGLVKPSETLSLT | |
| | | CTVSGVSLPDYGVSWIRQPPGKGLEWIGVIWGSETTY | |
| | | YSSSLKSRVTISKDNSKNQVSLKLSSVTAADTAVYYC | |
| | | AKHYYYGGSYAMDYWGQGTLVTVSS | |
| CD19-scFv6 | scFv | EIVMTQSPATLSLSPGERATLSCRASQDISKYLNWYQ | 1363 |
| | | QKPGQAPRLLIYHTSRLHSGIPARFSGSGSGTDYTLT | |
| | | ISSLQPEDFAVYFCQQGNTLPYTFGQGTKLEIKGGGG | |
| | | SGGGGSGGGGSGGGGSQVQLQESGPGLVKPSETLSLT | |
| | | CTVSGVSLPDYGVSWIRQPPGKGLEWIGVIWGSETTY | |
| | | YQSSLKSRVTISKDNSKNQVSLKLSSVTAADTAVYYC | |
| | | AKHYYYGGSYAMDYWGQGTLVTVSS | |
| CD19-scFv7 | scFv | QVQLQESGPGLVKPSETLSLTCTVSGVSLPDYGVSWI | 1364 |
| | | RQPPGKGLEWIGVIWGSETTYYSSSLKSRVTISKDNS | |
| | | NKQVSLKLSSVTAADTAVYYCAKHYYYGGSYAMDYWG | |
| | | QGTLVTVSSGGGGSGGGGSGGGGSGGGGSEIVMTQSP | |
| | | ATLSLSPGERATLSCRASQDISKYLNWYQQKPGQAPR | |
| | | LLIYHTSRLHSGIPARFSGSGSGTDYTLTISSLQPED | |
| | | FAVYFCQQGNTLPYTFGQGTKLEIK | |
| CD19-scFv8 | scFv | QVQLQESGPGLVKPSETLSLTCTVSGVSLPDYGVSWI | 1365 |
| | | RQPPGKGLEWIGVIWGSETTYYQSSLKSRVTISKDNS | |
| | | KNQVSLKLSSVTAADTAVYYCAKHYYYGGSYAMDYWG | |
| | | QGTLVTVSSGGGGSGGGGSGGGGSGGGGSEIVMTQSP | |
| | | ATLSLSPGERATLSCRASQDISKYLNWYQQKPGQAPR | |
| | | LLIYHTSRLHSGIPARFSGSGSGTDYTLTISSLQPED | |
| | | FAVYFCQQGNTLPYTFGQGTKLEIK | |
| CD19-scFv9 | scFv | EIVMTQSPATLSLSPGERATLSCRASQDISKYLNWYQ | 1366 |
| | | QKPGQAPRLLIYHTSRLHSGIPARFSGSGSGTDYTLT | |
| | | ISSLQPEDFAVYFCQQGNTLPYTFGQGTKLEIKGGGG | |
| | | SGGGGSGGGGSGGGGSQVQLQESGPGLVKPSETLSLT | |
| | | CTVSGVSLPDYGVSWIRQPPGKGLEWIGVIWGSETTY | |
| | | YNSSLKSRVTISKDNSKNQVSLKLSSVTAADTAVYYC | |
| | | AKHYYYGGSYAMDYWGQGTLVTVSS | |
| CD19-scFv10 | scFv | QVQLQESGPGLVKPSETLSLTCTVSGVSLPDYGVSWI | 1367 |
| | | RQPPGKGLEWIGVIWGSETTYYNSSLKSRVTISKDNS | |
| | | KNQVSLKLSSVTAADTAVYYCAKHYYYGGSYAMDYWG | |
| | | QGTLVTVSSGGGGSGGGGSGGGGSGGGGSEIVMTQSP | |
| | | ATLSLSPGERATLSCRASQDISKYLNWYQQKPGQAPR | |
| | | LLIYHTSRLHSGIPARFSGSGSGTDYTLTISSLQPED | |
| | | FAVYFCQQGNTLPYTFGQGTKLEIK | |
| CD19-scFv11 | scFv | EIVMTQSPATLSLSPGERATLSCRASQDISKYLNWYQ | 1368 |
| | | QKPGQAPRLLIYHTSRLHSGIPARFSGSGSGTDYTLT | |
| | | ISSLQPEDFAVYFCQQGNTLPYTFGQGTKLEIKGGGG | |
| | | SGGGGSGGGGSQVQLQESGPGLVKPSETLSLTCTVSG | |
| | | VSLPDYGVSWIRQPPGKGLEWIGVIWGSETTYYNSSL | |
| | | KSRVTISKDNSKNQVSLKLSSVTAADTAVYYCAKHYY | |
| | | YGGSYAMDYWGQGTLVTVSS | |
| CD19-scFv12 | scFv | QVQLQESGPGLVKPSETLSLTCTVSGVSLPDYGVSWI | 1369 |
| | | RQPPGKGLEWIGVIWGSETTYYNSSLKSRVTISKDNS | |
| | | KNQVSLKLSSVTAADTAVYYCAKHYYYGGSYAMDYWG | |
| | | QGTLVTVSSGGGGSGGGGSGGGGSEIVMTQSPATLSL | |
| | | SPGERATLSCRASQDISKYLNWYQQKPGQAPRLLIYH | |
| | | TSRLHSGIPARFSGSGSGTDYTLTISSLQPEDFAVYF | |
| | | CQQGNTLPYTFGQGTKLEIK | |
| CD19-scFv13 | scFv | DIQMTQTTSSLSASLGDRVTISCRASQDISKYLNWYQ | 1370 |
| | | QKPDGTVKLLIYHTSRLHSGVPSRFSGSGSGTDYSLT | |
| | | ISNLEQEDIATYFCQQGNTLPYTFGGGTKLEITGGGG | |
| | | SGGGGSGGGGSEVKLQESGPGLVAPSQSLSVTCTVSG | |
| | | VSLPDYGVSWIRQPPRKGLEWLGVIWGSETTYYNSAL | |
| | | KSRLTIIKDNSKSQVFLKMNSLQTDDTAIYYCAKHYY | |
| | | YGGSYAMDYWGQGTSVTVSS | |
| CD19-scFv14 | scFv | QVQLLESGAELVRPGSSVKISCKASGYAFSSYWMNWV | 1371 |
| | | KQRPGQGLEWIGQIYPGDGDTNYNGKFKGQATLTADK | |
| | | SSSTAYMQLSGLTSEDSAVYSCARKTISSVVDFYFDY | |
| | | WGQGTTVTGGGSGGGSGGGSGGGSELVLTQSPKFMST | |
| | | SVGDRVSVTCKASQNVGTNVAWYQQKPGQSPKPLIYS | |
| | | ATYRNSGVPDRFTGSGSGTDFTLTITNVQSKDLADYF | |
| | | CQYNRYPYTSFFFTKLEIKRRS | |
| CD19-scFv15 | scFv | DIQMTQTTSSLSASLGDRVTISCRASQDISKYLNWYQ | 1372 |
| | | QKPDGTVKLLIYHTSRLHSGVPSRFSGSGSGTDYSLT | |
| | | ISNLEQEDIATYFCQQGNTLPYTFGGGTKLEITGSTS | |
| | | GSGKPGSGEGSTKGEVKLQESGPGLVAPSQSLSVTCT | |
| | | VSGVSLPDYGVSWIRQPPRKGLEWLGVIWGSETTYYN | |
| | | SALKSRLTIIKDNSKSQVFLKMNSLQTDDTAIYYCAK | |
| | | HYYYGGSYAMDYWGQGTSVTVSSE | |
| CD19-scFv16 | scFv | DIQMTQTTSSLSASLGDRVTISCRASQDISKYLNWYQ | 1373 |
| | | QKPDGTVKLLIYHTSRLHSGVPSRFSGSGSGTDYSLT | |
| | | ISNLEQEDIATYFCQQGNTLPYTFGGGTKLEITGSTS | |
| | | GSGKPGSGEGSTKGEVKLQESGPGLVAPSQSLSVTCT | |
| | | VSGVSLPDYGVSWIRQPPRKGLEWLGVIWGSETTYYN | |
| | | SALKSRLTIIKDNSKSQVFLKMNSLQTDDTAIYYCAK | |
| | | HYYYGGSYAMDYWGQGTSVTVSS | |

In certain aspects, a CD19 ABM (e.g., ABM2 or ABM5) comprises heavy chain CDRs having the amino acid sequences of CD19-H1, CD19-H2A, and CD19-H3 as set forth in Table 16 and light chain CDRs having the amino acid sequences of CD19-L1, CD19-L2, and CD19-L3 as set forth in Table 16. In a specific embodiment, the ABM comprises a heavy chain variable region having the amino acid sequences of VHA as set forth in Table 16 and a light chain variable region having the amino acid sequences of VLA as set forth in Table 16.

In other aspects, the ABM comprises heavy chain CDRs having the amino acid sequences of CD19-H1, CD19-H2B, and CD19-H3 as set forth in Table 16 and light chain CDRs having the amino acid sequences of CD19-L1, CD19-L2, and CD19-L3 as set forth in Table 16. In a specific embodiment, the ABM comprises a heavy chain variable region having the amino acid sequences of VHB as set forth in Table 16 and a light chain variable region having the amino acid sequences of VLB as set forth in Table 16.

In further aspects, the ABM comprises heavy chain CDRs having the amino acid sequences of CD19-H1, CD19-H2C, and CD19-H3 as set forth in Table 16 and light chain CDRs having the amino acid sequences of CD19-L1, CD19-L2, and CD19-L3 as set forth in Table 16. In a specific embodiment, ABM comprises a heavy chain variable region having the amino acid sequences of VHC as set forth in Table 16 and a light chain variable region having the amino acid sequences of VLB as set forth in Table 16.

In further aspects, the ABM comprises heavy chain CDRs having the amino acid sequences of CD19-H1, CD19-H2D, and CD19-H3 as set forth in Table 16 and light chain CDRs having the amino acid sequences of CD19-L1, CD19-L2, and CD19-L3 as set forth in Table 16. In a specific embodiment, the ABM comprises a heavy chain variable region having the amino acid sequences of VHD as set forth in Table 16 and a light chain variable region having the amino acid sequences of VLB as set forth in Table 16.

In further aspects, the ABM comprises a heavy chain variable region having the amino acid sequences of VHE as set forth in Table 16 and a light chain variable region having the amino acid sequence of VLE as set forth in Table 16.

In yet further aspects, the ABM is in the form of an scFV. Exemplary anti-CD19 scFvs comprise the amino acid sequence of any one of CD19-scFv1 through CD19-scFv16 as set forth in Table 16.

Tables 17A and 17B (collectively “Table 17”) list the sequences of additional exemplary CD19 binding sequences that can be included in a CD19 ABM. The sequences set forth in Table 17A are based on the CD19 antibody NEG258.

TABLE 17A
NEG258-Based Binder Sequences

| | | | SEQ ID |
|---|---|---|---|
| Chain | Portion | Sequence | NO: |
| NEG258_VH | CDR-H1 | GYTFTTYWIQ | 1 |
| | (Combined) | | |
| | CDR-H2 | AVYPGDADTRYTQKFQG | 2 |
| | (Combined) | | |
| | CDR-H3 | DAGLEYYALDY | 3 |
| | (Combined) | | |
| | CDR-H1 (Kabat) | TYWIQ | 4 |
| | CDR-H2 (Kabat) | AVYPGDADTRYTQKFQG | 5 |
| | CDR-H3 (Kabat) | DAGLEYYALDY | 6 |
| | CDR-H1 | GYTFTTY | 7 |
| | (Chothia) | | |
| | CDR-H2 | YPGDAD | 8 |
| | (Chothia) | | |
| | CDR-H3 | DAGLEYYALDY | 9 |
| | (Chothia) | | |
| | CDR-H1 (IMGT) | GYTFTTYW | 10 |
| | CDR-H2 (IMGT) | VYPGDADT | 11 |
| | CDR-H3 (IMGT) | GRDAGLEYYALDY | 12 |
| | VH | QVQLVQSGAEVKKPGASVKVSCKASGYTFTTYWIQW | 13 |
| | | VRQAPGQRLEWMGAVYPGDADTRYTQKFQGRVTLTA | |
| | | DRSASTAYMELSSLRSEDTAVYYCGRDAGLEYYALD | |
| | | YWGQGTLVTVSS | |
| NEG258_VL | CDR-L1 | RASQDVGTAVA | 14 |
| | (Combined) | | |
| | CDR-L2 | WASTRHT | 15 |
| | (Combined) | | |
| | CDR-L3 | QQYANFPLYT | 16 |
| | (Combined) | | |
| | CDR-L1 (Kabat) | RASQDVGTAVA | 17 |
| | CDR-L2 (Kabat) | WASTRHT | 18 |
| | CDR-L3 (Kabat) | QQYANFPLYT | 19 |
| | CDR-L1 | SQDVGTA | 20 |
| | (Chothia) | | |
| | CDR-L2 | WAS | 21 |
| | (Chothia) | | |
| | CDR-L3 | YANFPLY | 22 |
| | (Chothia) | | |
| | CDR-L1 (IMGT) | QDVGTA | 23 |
| | CDR-L2 (IMGT) | WAS | 24 |
| | CDR-L3 (IMGT) | QQYANFPLYT | 25 |
| | VL | EIVMTQSPATLSVSPGERATLSCRASQDVGTAVAWY | 26 |
| | | QQKPGQAPRLLIYWASTRHTGIPARFSGSGSGTEFT | |
| | | LTISSLQSEDFAVYFCQQYANFPLYTFGQGTKLEIK | |

In some embodiments, a CD19 ABM comprises CDR-L1, CDR-L2, CDR-L3, CDR-H1, CDR-H2 and CDR-H3 sequences of NEG258 as set forth in Table 17A. The CDR-L1, CDR-L2, CDR-L3, CDR-H1, CDR-H2 and CDR-H3 sequences can be as defined by Kabat, Chothia, or IMGT, or the combined Chothia and Kabat CDR-L1, CDR-L2, CDR-L3, CDR-H1, CDR-H2 and CDR-H3 sequences. The CD19 ABM can also comprise a light chain variable sequence and/or heavy chain variable sequence of the anti-CD19 antibody NEG258 as set forth in Table 17A.

The sequences set forth in Table 17B are based on the CD19 antibody NEG218.

TABLE 17B
NEG218-Based Sequences

| | | | SEQ ID |
|---|---|---|---|
| Chain | Portion | Sequence | NO: |
| NEG218_VH | CDR-H1 | GYSFTNYWMN | 27 |
| | (Combined) | | |
| | CDR-H2 | MIHPSDSEIRLNQKFQG | 28 |
| | (Combined) | | |
| | CDR-H3 | WYYLSSPMDY | 29 |
| | (Combined) | | |
| | CDR-H1 (Kabat) | NYWMN | 30 |
| | CDR-H2 (Kabat) | MIHPSDSEIRLNQKFQG | 31 |
| | CDR-H3 (Kabat) | WYYLSSPMDY | 32 |
| | CDR-H1 | GYSFTNY | 33 |
| | (Chothia) | | |
| | CDR-H2 | HPSDSE | 34 |
| | (Chothia) | | |
| | CDR-H3 | WYYLSSPMDY | 35 |
| | (Chothia) | | |
| | CDR-H1 (IMGT) | GYSFTNYW | 36 |
| | CDR-H2 (IMGT) | IHPSDSEI | 37 |
| | CDR-H3 (IMGT) | SRWYYLSSPMDY | 38 |
| | VH | EVQLVQSGAEVKKPGESLKISCKASGYSFTNYWMNW | 39 |
| | | VRQMPGKGLEWMGMIHPSDSEIRLNQKFQGQVTLSV | |
| | | DKSIGTAYMQWSSLKASDTAMYYCSRWYYLSSPMDY | |
| | | WGQGTTVTVSS | |
| NEG218_VL | CDR-L1 | RASQDVGTAVA | 40 |
| | (Combined) | | |
| | CDR-L2 | WASTRHT | 41 |
| | (Combined) | | |
| | CDR-L3 | QQYSSYPYT | 42 |
| | (Combined) | | |
| | CDR-L1 (Kabat) | RASQDVGTAVA | 43 |
| | CDR-L2 (Kabat) | WASTRHT | 44 |
| | CDR-L3 (Kabat) | QQYSSYPYT | 45 |
| | CDR-L1 | SQDVGTA | 46 |
| | (Chothia) | | |
| | CDR-L2 | WAS | 47 |
| | (Chothia) | | |
| | CDR-L3 | YSSYPY | 48 |
| | (Chothia) | | |
| | CDR-L1 (IMGT) | QDVGTA | 49 |
| | CDR-L2 (IMGT) | WAS | 50 |
| | CDR-L3 (IMGT) | QQYSSYPYT | 51 |
| | VL | EIVMTQSPATLSVSPGERATLSCRASQDVGTAVAWY | 52 |
| | | QQKPGQAPRLLIYWASTRHTGIPARFSGSGSGTEFT | |
| | | LTISSLQSEDFAVYFCQQYSSYPYTFGQGTKLEIK | |

In some embodiments, a CD19 ABM (e.g., ABM2 or ABM5) comprises CDR-L1, CDR-L2, CDR-L3, CDR-H1, CDR-H2 and CDR-H3 sequences of NEG218 as set forth in Table 17B. The CDR-L1, CDR-L2, CDR-L3, CDR-H1, CDR-H2 and CDR-H3 sequences can be as defined by Kabat, Chothia, or IMGT, or the combined Chothia and Kabat CDR-L1, CDR-L2, CDR-L3, CDR-H1, CDR-H2 and CDR-H3 sequences. The CD19 ABM can also comprise a light chain variable sequence and/or heavy chain variable sequence of the anti-CD19 antibody NEG218 as set forth in Table 17B.

Other CD19 ABMs include amino acids that have been mutated, yet have at least 80, 85, 90, 95, 96, 97, 98, or 99 percent identity in the CDR regions with the CDR sequences described in Table 17. In some embodiments, such CD19 ABMs include mutant amino acid sequences where no more than 1, 2, 3, 4 or 5 amino acids have been mutated in the CDR regions when compared with the CDR sequences described in Table 17.

Other CD19 ABMs include VH and/or VL domains comprising amino acid sequences having at least 80, 85, 90, 95, 96, 97, 98, or 99 percent identity to the VH and/or VL sequences described in Table 17. In some embodiments, CD19 binding molecules include VH and/or VL domains where no more than 1, 2, 3, 4 or 5 amino acids have been mutated when compared with the VH and/or VL domains depicted in the sequences described in Table 17, while retaining substantially the same therapeutic activity.

### 7.7.1. CD20

In certain aspects, the present disclosure provides MBMs and combinations of MBMs in which ABM2 and/or ABM5 binds specifically to CD20. CD20 is expression is associated with B-cell lymphomas, hairy cell leukemia, B-cell chronic lymphocytic leukemia, and melanoma.

A CD20 ABM (e.g., ABM2 and/or ABM5) can comprise, for example, an anti-CD20 antibody or an antigen-binding domain thereof. The anti-CD20 antibody or antigen-binding domain thereof can comprise, for example, CDR or VH and/or VL sequences set forth in Table 18.

TABLE 18
CD20 Binders

| | | | SEQ ID |
|---|---|---|---|
| Binder | Domain | Sequence | NO: |
| CD20_1 | CDR-H1 | GYTFTSYNMH | 1374 |
| | (Combined | | |
| | Kabat + Chothia) | | |
| | CDR-H2 | AIYPGNGDTSYNQKFKG | 1375 |
| | (Combined | | |
| | Kabat + Chothia) | | |
| | CDR-H3 | STYYGGDWYFNV | 1376 |
| | (Combined | | |
| | Kabat + Chothia) | | |
| | CDR-H1 (Kabat) | SYNMH | 1377 |
| | CDR-H2 (Kabat) | AIYPGNGDTSYNQKFKG | 1375 |
| | CDR-H3 (Kabat) | STYYGGDWYFNV | 1376 |
| | CDR-H1 | GYTFTSY | 265 |
| | (Chothia) | | |
| | CDR-H2 | YPGNGD | 1378 |
| | (Chothia) | | |
| | CDR-H3 | STYYGGDWYFNV | 1376 |
| | (Chothia) | | |
| | CDR-H1 (IMGT) | GYTFTSYN | 1379 |
| | CDR-H2 (IMGT) | YPGNGDT | 1380 |
| | CDR-H3 (IMGT) | ARSTYYGGDWYFNV | 1381 |
| | VH | QVQLQQPGAELVKPGASVKMSCKASGYTFTS | 1382 |
| | | YNMHWWKQTPGRGLEWIGAIYPGNGDTSYNQ | |
| | | KFKGKATLTADKSSSTAYMQLSSLTSEDSAV | |
| | | YYCARSTYYGGDWYFNVWGAGTTVTVSA | |
| | CDR-L1 | RASSSVSYIH | 1383 |
| | (Combined | | |
| | Kabat + Chothia) | | |
| | CDR-L2 | ATSNLAS | 217 |
| | (Combined | | |
| | Kabat + Chothia) | | |
| | CDR-L3 | QQWTSNPPT | 1384 |
| | (Combined | | |
| | Kabat + Chothia) | | |
| | CDR-L1 (Kabat) | RASSSVSYIH | 1383 |
| | CDR-L2 (Kabat) | ATSNLAS | 217 |
| | CDR-L3 (Kabat) | QQWTSNPPT | 1384 |
| | CDR-L1 (Chothia) | SSSVSY | 262 |
| | CDR-L2 (Chothia) | ATS | 279 |
| | CDR-L3 (Chothia) | WTSNPP | 1385 |
| | CDR-L1 (IMGT) | SSVSY | 1386 |
| | CDR-L2 (IMGT) | ATS | 279 |
| | CDR-L3 (IMGT) | QQWTSNPPT | 1384 |
| | VL | QIVLSQSPAILSASPGEKVTMTCRASSSVSY | 1387 |
| | | IHWFQQKPGSSPKPWIYATSNLASGVPVRFS | |
| | | GSGSGTSYSLTISRVEAEDAATYYCQQWTSN | |
| | | PPTFGGGTKLEIK | |

In some embodiments, a CD20 ABM comprises Kabat CDR-H1, CDR-H2, CDR-H3, CDR-L1, CDR-L2, and CDR-L3 sequences of CD20_1 as set forth in Table 18. In some embodiments, a CD20 ABM comprises Chothia CDR-H1, CDR-H2, CDR-H3, CDR-L1, CDR-L2, and CDR-L3 sequences of CD20_1 as set forth in Table 18. In some embodiments, a CD20 ABM comprises IMGT CDR-H1, CDR-H2, CDR-H3, CDR-L1, CDR-L2, and CDR-L3 sequences of CD20L1 as set forth in Table 18. In some embodiments, a CD20 ABM comprises combined Kabat+Chothia CDR-H1, CDR-H2, CDR-H3, CDR-L1, CDR-L2, and CDR-L3 sequences of CD20_1 as set forth in Table 18. In some embodiments, a CD20 ABM comprises VH and/or VL sequences of CD20_1 as set forth in Table 18.

### 7.7.1. CD22

In certain aspects, the present disclosure provides MBMs and combinations of MBMs in which ABM2 and/or ABM5 binds specifically to CD22. CD22 is found on mature B cells, and is widely expressed on B-cell leukemias and lymphomas.

A CD22 ABM (e.g., ABM2 and/or ABM5) can comprise, for example, an anti-CD22 antibody or an antigen-binding domain thereof. The anti-CD22 antibody or antigen-binding domain thereof can comprise, for example, CDR or VH and/or VL sequences set forth in Table 19.

TABLE 19
CD22 Binders

| | | | SEQ |
|---|---|---|---|
| | | | ID |
| Binder | Domain | Sequence | NO: |
| CD22_HA22 | CDR-H1 | GFAFSIYDMS | 1388 |
| | (Combined | | |
| | Kabat + Chothia) | | |
| | CDR-H2 | YISSGGGTYYPDTVKG | 1389 |
| | (Combined | | |
| | Kabat + Chothia) | | |
| | CDR-H3 | HSGYGTHWGVLFAY | 1390 |
| | (Combined | | |
| | Kabat + Chothia) | | |
| | CDR-H1 (Kabat) | YDMS | 1391 |
| | CDR-H2 (Kabat) | YISSGGGTYYPDTVKG | 1389 |
| | CDR-H3 (Kabat) | HSGYGTHWGVLFAY | 1390 |
| | CDR-H1 | GFAFSIY | 1392 |
| | (Chothia) | | |
| | CDR-H2 | SSGGG | 1393 |
| | (Chothia) | | |
| | CDR-H3 | HSGYGTHWGVLFAY | 1390 |
| | (Chothia) | | |
| | CDR-H1 (IMGT) | GFAFSIYD | 1394 |
| | CDR-H2 (IMGT) | ISSGGGT | 1395 |
| | CDR-H3 (IMGT) | ARHSGYGTHWGVLFAY | 1396 |
| | VH | EVQLVESGGGLVKPGGSLKLSCAASGFAFSI | 1397 |
| | | YDMSWVRQTPEKRLEVWAYiSSGGGTYYPDT | |
| | | VKGRFTISRDNAKNTLYLQMSSLKSEDTAMY | |
| | | YCARHSGYGTHWGVLFAYWGQGTLVTVSA | |
| | CDR-L1 | RASQDISNYLN | 1398 |
| | (Combined | | |
| | Kabat + Chothia) | | |
| | CDR-L2 | YTSILHS | 1399 |
| | (Combined | | |
| | Kabat + Chothia) | | |
| | CDR-L3 | QQGNTLPWT | 253 |
| | (Combined | | |
| | Kabat + Chothia) | | |
| | CDR-L1 (Kabat) | RASQDISNYLN | 1398 |
| | CDR-L2 (Kabat) | YTSILHS | 1399 |
| | CDR-L3 (Kabat) | QQGNTLPWT | 253 |
| | CDR-L1 (Chothia) | SQDISNY | 1400 |
| | CDR-L2 (Chothia) | YTS | 285 |
| | CDR-L3 (Chothia) | GNTLPW | 294 |
| | CDR-L1 (IMGT) | QDISNY | 1401 |
| | CDR-L2 (IMGT) | YTSILHSGVP | 1402 |
| | CDR-L3 (IMGT) | QQGNTLPWT | 253 |
| | VL | DIQMTQTTSSLSASLGDRVTISCRASQDISN | 1403 |
| | | YLNWWYQQKPDGTVKLLIYYTSILHSGVPSR | |
| | | FSGSGSGTDYSLTISNLEQEDFATYFCQQGN | |
| | | TLPWTFGGGTKLEIK | |
| CD22_m971 | CDR-H1 | GDSVSSNSAAWN | 742 |
| | (Combined | | |
| | Kabat + Chothia) | | |
| | CDR-H2 | RTYYRSKWYNDYAVSVKS | 1404 |
| | (Combined | | |
| | Kabat + Chothia) | | |
| | CDR-H3 | EVTGDLEDAFDI | 1405 |
| | (Combined | | |
| | Kabat + Chothia) | | |
| | CDR-H1 (Kabat) | SNSAAWN | 632 |
| | CDR-H2 (Kabat) | RTYYRSKWYNDYAVSVKS | 1404 |
| | CDR-H3 (Kabat) | EVTGDLEDAFDI | 1405 |
| | CDR-H1 | GDSVSSNSA | 694 |
| | (Chothia) | | |
| | CDR-H2 | YYRSKW | 714 |
| | (Chothia) | | |
| | CDR-H3 | EVTGDLEDAFDI | 1405 |
| | (Chothia) | | |
| | CDR-H1 (IMGT) | GDSVSSNSAA | 1406 |
| | CDR-H2 (IMGT) | TYYRSKWYN | 1407 |
| | CDR-H3 (IMGT) | AREVTGDLEDAFDI | 1408 |
| | VH | QVQLQQSGPGLVKPSQTLSLTCAISGDSVSS | 1409 |
| | | NSAAWNWIRQSPSRGLEWLGRTYYRSKWYND | |
| | | YAVSVKSRITINPDTSKNQFSLQLNSVTPED | |
| | | TAVYYCAREVTGDLEDAFDIWGQGTMVTVSS | |
| | CDR-L1 | RASQTIWSYLN | 1410 |
| | (Combined | | |
| | Kabat + Chothia) | | |
| | CDR-L2 | AASSLQS | 482 |
| | (Combined | | |
| | Kabat + Chothia) | | |
| | CDR-L3 | QQSYSIPQT | 1411 |
| | (Combined | | |
| | Kabat + Chothia) | | |
| | CDR-L1 (Kabat) | RASQTIWSYLN | 1410 |
| | CDR-L2 (Kabat) | AASSLQS | 482 |
| | CDR-L3 (Kabat) | QQSYSIPQT | 1411 |
| | CDR-L1 (Chothia) | SQTIWSY | 1412 |
| | CDR-L2 (Chothia) | AAS | 577 |
| | CDR-L3 (Chothia) | SYSIPQ | 1413 |
| | CDR-L1 (IMGT) | QTIWSY | 1414 |
| | CDR-L2 (IMGT) | AAS | 577 |
| | CDR-L3 (IMGT) | QQSYSIPQT | 1411 |
| | VL | DIQMTQSPSSLSASVGDRVTITCRASQTIWS | 1415 |
| | | YLNWYQQRPGKAPNLLIYAASSLQSGVPSRF | |
| | | SGRGSGTDFTLTISSLQAEDFATYYCQQSYS | |
| | | IPQTFGQGTKLEIK | |
| CD22_65 | CDR-H1 | GDSMLSNSDTWN | 1416 |
| | (Combined | | |
| | Kabat + Chothia) | | |
| | CDR-H2 | RTYHRSTWDDYASSVRG | 1417 |
| | (Combined | | |
| | Kabat + Chothia) | | |
| | CDR-H3 | VRLQDGNSWSDAFDV | 1418 |
| | (Combined | | |
| | Kabat + Chothia) | | |
| | CDR-H1 (Kabat) | CAISGDSMLSNSDTWN | 1419 |
| | CDR-H2 (Kabat) | RTYHRSTWYDDYASSVRG | 1417 |
| | CDR-H3 (Kabat) | VRLQDGNSWSDAFDV | 1418 |
| | CDR-H1 | GDSMLSNSD | 1420 |
| | (Chothia) | | |
| | CDR-H2 | YHRSTWY | 1421 |
| | (Chothia) | | |
| | CDR-H3 | VRLQDGNSWSDAFDV | 1418 |
| | (Chothia) | | |
| | CDR-H1 (IMGT) | GDSMLSNSDT | 1422 |
| | CDR-H2 (IMGT) | TYHRSTWD | 1423 |
| | CDR-H3 (IMGT) | ARVRLQDGNSWSDAFDV | 1424 |
| | VH | EVQLQQSGPGLVKPSQTLSLTCAISGDSMLS | 1425 |
| | | NSDTWNWIRQSPSRGLEWLGRTYHRSTWYDD | |
| | | YASSVRGRVSINVDTSKNQYSLQLNAVTPED | |
| | | TGVYYCARVRLQDGNSWSDAFDVWGQGTMVT | |
| | | VSS | |
| | CDR-L1 | TGTSSDVGGYNYVS | 1094 |
| | (Combined | | |
| | Kabat + Chothia) | | |
| | CDR-L2 | DVSNRPS | 1166 |
| | (Combined | | |
| | Kabat + Chothia) | | |
| | CDR-L3 | SSYTSSSTLYV | 1174 |
| | (Combined | | |
| | Kabat + Chothia) | | |
| | CDR-L1 (Kabat) | TGTSSDVGGYNYVS | 1094 |
| | CDR-L2 (Kabat) | DVSNRPS | 1166 |
| | CDR-L3 (Kabat) | SSYTSSSTLYV | 1174 |
| | CDR-L1 (Chothia) | TSSDVGGYNY | 1095 |
| | CDR-L2 (Chothia) | DVS | 1099 |
| | CDR-L3 (Chothia) | YTSSSTLY | 1199 |
| | CDR-L1 (IMGT) | SSDVGGYNY | 1096 |
| | CDR-L2 (IMGT) | DVS | 1099 |
| | CDR-L3 (IMGT) | SSYTSSSTLYV | 1174 |
| | VL | QSALTQPASASGSPGQSVTISCTGTSSDVGG | 1433 |
| | | YNYVSWYQQHPGKAPKLMIYDVSNRPSGVSN | |
| | | RFSGSKSGNTASLTiSGLQAEDEADYYCSSY | |
| | | TSSSTLYVFGTGTQLTVL | |

In some embodiments, a CD22 ABM comprises Kabat CDR-H1, CDR-H2, CDR-H3, CDR-L1, CDR-L2, and CDR-L3 sequences of CD22_HA22 as set forth in Table 19. In some embodiments, a CD22 ABM comprises Chothia CDR-H1, CDR-H2, CDR-H3, CDR-L1, CDR-L2, and CDR-L3 sequences of CD22_HA22 as set forth in Table 19. In some embodiments, a CD22 ABM comprises IMGT CDR-H1, CDR-H2, CDR-H3, CDR-L1, CDR-L2, and CDR-L3 sequences of CD22_HA22 as set forth in Table 19. In some embodiments, a CD22 ABM comprises combined Kabat+Chothia CDR-H1, CDR-H2, CDR-H3, CDR-L1, CDR-L2, and CDR-L3 sequences of CD22_HA22 as set forth in Table 19. In some embodiments, a CD22 ABM comprises VH and/or VL sequences of CD22_HA22 as set forth in Table 19.

In some embodiments, a CD22 ABM comprises Kabat CDR-H1, CDR-H2, CDR-H3, CDR-L1, CDR-L2, and CDR-L3 sequences of CD22_m971 as set forth in Table 19. In some embodiments, a CD22 ABM comprises Chothia CDR-H1, CDR-H2, CDR-H3, CDR-L1, CDR-L2, and CDR-L3 sequences of CD22_m971 as set forth in Table 19. In some embodiments, a CD22 ABM comprises IMGT CDR-H1, CDR-H2, CDR-H3, CDR-L1, CDR-L2, and CDR-L3 sequences of CD22_m971 as set forth in Table 19. In some embodiments, a CD22 ABM comprises combined Kabat+Chothia CDR-H1, CDR-H2, CDR-H3, CDR-L1, CDR-L2, and CDR-L3 sequences of CD22_m971 as set forth in Table 19. In some embodiments, a CD22 ABM comprises VH and/or VL sequences of CD22_m971 as set forth in Table 19.

In some embodiments, a CD22 ABM comprises Kabat CDR-H1, CDR-H2, CDR-H3, CDR-L1, CDR-L2, and CDR-L3 sequences of CD22_65 as set forth in Table 19. In some embodiments, a CD22 ABM comprises Chothia CDR-H1, CDR-H2, CDR-H3, CDR-L1, CDR-L2, and CDR-L3 sequences of CD22_65 as set forth in Table 19. In some embodiments, a CD22 ABM comprises IMGT CDR-H1, CDR-H2, CDR-H3, CDR-L1, CDR-L2, and CDR-L3 sequences of CD22_65 as set forth in Table 19. In some embodiments, a CD22 ABM comprises combined Kabat+Chothia CDR-H1, CDR-H2, CDR-H3, CDR-L1, CDR-L2, and CDR-L3 sequences of CD22_65 as set forth in Table 19. In some embodiments, a CD22 ABM comprises VH and/or VL sequences of CD22_65 as set forth in Table 19.

### 7.7.2. Mesothelin

In certain aspects, the present disclosure provides MBMs and combinations of MBMs in which ABM2 and/or ABM5 binds specifically to mesothelin (MSLN). Mesothelin is over expressed in several cancers, including mesothelioma, ovarian cancer, pancreatic adenocarcinoma, lung adenocarcinoma, and cholangiocarcinoma.

A Mesothelin ABM can comprise, for example, an anti-mesothelin antibody or an antigen-binding domain thereof. The anti-mesothelin antibody or antigen-binding domain thereof can comprise, for example, CDR or VH and/or VL sequences set forth in Table 20.

TABLE 20
Mesothelin Binders

| | | | SEQ |
|---|---|---|---|
| | | | ID |
| Binder | Domain | Sequence | NO: |
| MSLN_SS1 | CDR-H1 (Combined | GYSFTGYTMN | 302 |
| | Kabat + Chothia) | | |
| | CDR-H2 (Combined | LITPYNGASSYNQKFRG | 1434 |
| | Kabat + Chothia) | | |
| | CDR-H3 (Combined | GGYDGRGFDY | 1435 |
| | Kabat + Chothia) | | |
| | CDR-H1 (Kabat) | GYTMN | 205 |
| | CDR-H2 (Kabat) | LITPYNGASSYNQKFRG | 1434 |
| | CDR-H3 (Kabat) | GGYDGRGFDY | 1435 |
| | CDR-H1 (Chothia) | GYSFTGY | 272 |
| | CDR-H2 (Chothia) | TPYNGA | 1436 |
| | CDR-H3 (Chothia) | GGYDGRGFDY | 1435 |
| | CDR-H1 (IMGT) | GYSFTGYT | 1437 |
| | CDR-H2 (IMGT) | ITPYNGAS | 1438 |
| | CDR-H3 (IMGT) | ARGGYDGRGFDY | 1439 |
| | VH | QVQLQQSGPELEKPGASVKISCKASGYSFTGYT | 1440 |
| | | MNWVKQSHGKSLEWiGLITPYNGASSYNQKFRG | |
| | | KATLTVDKSSSTAYMDLLSLTSEDSAVYFCARG | |
| | | GYDGRGFDYWGQGTTVTVSS | |
| | CDR-L1 (Combined | SASSSVSYMH | 1441 |
| | Kabat + Chothia) | | |
| | CDR-L2 (Combined | DTSKLAS | 213 |
| | Kabat + Chothia) | | |
| | CDR-L3 (Combined | QQWSGYPLT | 1442 |
| | Kabat + Chothia) | | |
| | CDR-L1 (Kabat) | SASSSVSYMH | 1441 |
| | CDR-L2 (Kabat) | DTSKLAS | 213 |
| | CDR-L3 (Kabat) | QQWSGYPLT | 1442 |
| | CDR-L1 (Chothia) | SSSVSY | 262 |
| | CDR-L2 (Chothia) | DTS | 275 |
| | CDR-L3 (Chothia) | WSGYPL | 1443 |
| | CDR-L1 (IMGT) | SSVSY | 1386 |
| | CDR-L2 (IMGT) | DTS | 275 |
| | CDR-L3 (IMGT) | QQWSGYPLT | 1442 |
| | VL | DIELTQSPAIMSASPGEKVTMTCSASSSVSYMH | 1444 |
| | | WYQQKSGTSPKRWIYDTSKLASGVPGRFSGSGS | |
| | | GNSYSLTISSVEAEDDATYYCQQWSGYPLTFGA | |
| | | GTKLEIK | |
| MSLN_M5 | CDR-H1 (Combined | GYTFTDYYMH | 1445 |
| | Kabat + Chothia) | | |
| | CDR-H2 (Combined | WINPNSGGTNYAQKFQG | 1446 |
| | Kabat + Chothia) | | |
| | CDR-H3 (Combined | GWDFDY | 1447 |
| | Kabat + Chothia) | | |
| | CDR-H1 (Kabat) | DYYMH | 1448 |
| | CDR-H2 (Kabat) | WINPNSGGTNYAQKFQG | 1446 |
| | CDR-H3 (Kabat) | GWDFDY | 1447 |
| | CDR-H1 (Chothia) | GYTFTDY | 698 |
| | CDR-H2 (Chothia) | NPNSGG | 1449 |
| | CDR-H3 (Chothia) | GWDFDY | 1447 |
| | CDR-H1 (IMGT) | GYTFTDYY | 1450 |
| | CDR-H2 (IMGT) | INPNSGGT | 1451 |
| | CDR-H3 (IMGT) | ASGWDFDY | 1452 |
| | VH | QVQLVQSGAEVEKPGASVKVSCKASGYTFTDYY | 1453 |
| | | MHWWRQAPGQGLEWMGWINPNSGGTNYAQKFQG | |
| | | RVTMTRDTSISTAYMELSRLRSDDTAVYYCASG | |
| | | WDFDYWGQGTLVTVSS | |
| | CDR-L1 (Combined | RASQSIRYYLS | 1454 |
| | Kabat + Chothia) | | |
| | CDR-L2 (Combined | TASILQN | 1455 |
| | Kabat + Chothia) | | |
| | CDR-L3 (Combined | LQTYTTPD | 1456 |
| | Kabat + Chothia) | | |
| | CDR-L1 (Kabat) | RASQSIRYYLS | 1454 |
| | CDR-L2 (Kabat) | TASILQN | 1455 |
| | CDR-L3 (Kabat) | LQTYTTPD | 1456 |
| | CDR-L1 (Chothia) | SQSIRYY | 1457 |
| | CDR-L2 (Chothia) | TAS | 1458 |
| | CDR-L3 (Chothia) | TYTTP | 1459 |
| | CDR-L1 (IMGT) | QSIRYY | 1460 |
| | CDR-L2 (IMGT) | TASILQNGVP | 1461 |
| | CDR-L3 (IMGT) | LQTYTTPD | 1456 |
| | VL | DIVMTQSPSSLSASVGDRVTITCRASQSIRYYL | 1462 |
| | | SWYQQKPGKAPKLLIYTASiLQNGVPSRFSGSG | |
| | | SGTDFTLTISSLQPEDFATYYCLQTYTTPDFGP | |
| | | GTKVEK | |

In some embodiments, a MSLN ABM comprises Kabat CDR-H1, CDR-H2, CDR-H3, CDR-L1, CDR-L2, and CDR-L3 sequences of MSLN_SS1 as set forth in Table 20. In some embodiments, a MSLN ABM comprises Chothia CDR-H1, CDR-H2, CDR-H3, CDR-L1, CDR-L2, and CDR-L3 sequences of MSLN_SS1 as set forth in Table 20. In some embodiments, a MSLN ABM comprises IMGT CDR-H1, CDR-H2, CDR-H3, CDR-L1, CDR-L2, and CDR-L3 sequences of MSLN_SS1 as set forth in Table 20. In some embodiments, a MSLN ABM comprises combined Kabat+Chothia CDR-H1, CDR-H2, CDR-H3, CDR-L1, CDR-L2, and CDR-L3 sequences of MSLN_SS1 as set forth in Table 20. In some embodiments, a MSLN ABM comprises VH and/or VL sequences of MSLN_SS1 as set forth in Table 20.

In some embodiments, a MSLN ABM comprises Kabat CDR-H1, CDR-H2, CDR-H3, CDR-L1, CDR-L2, and CDR-L3 sequences of MSLN_M5 as set forth in Table 20. In some embodiments, a MSLN ABM comprises Chothia CDR-H1, CDR-H2, CDR-H3, CDR-L1, CDR-L2, and CDR-L3 sequences of MSLN_M5 as set forth in Table 20. In some embodiments, a MSLN ABM comprises IMGT CDR-H1, CDR-H2, CDR-H3, CDR-L1, CDR-L2, and CDR-L3 sequences of MSLN_M5 as set forth in Table 20. In some embodiments, a MSLN ABM comprises combined Kabat+Chothia CDR-H1, CDR-H2, CDR-H3, CDR-L1, CDR-L2, and CDR-L3 sequences of MSLN_M5 as set forth in Table 20. In some embodiments, a MSLN ABM comprises VH and/or VL sequences of MSLN_M5 as set forth in Table 20.

### 7.8. Tumor Microenvironment Antigen ABMs

ABM3 of the first MBMs and ABM6 of the second MBMs of the disclosure, when present, bind specifically to a tumor microenvironment antigen (TMEA) (“TMEA 1” and “TMEA 2,” respectfully). In some combinations, only one of the first and second MBM has a ABM that binds to a TMEA. In other combinations, both the first and second MBM has a ABM that binds to a TMEA. In some of these combinations of first and second MBMs, TMEA 1 and TMEA 2 are the same. In other combinations of first and second MBMs in which both MBMs have a ABM that binds to a TMEA, TMEA 1 and TMEA 2 are different. When TMEA 1 and TMEA 2 are the same, ABM3 and ABM6 preferably bind to different epitopes on the TMEA (e.g., non-overlapping epitopes) so that the first MBM and the second MBM are able to specifically bind to the TMEA simultaneously. In some embodiments, ABM3 and ABM6 are selected so that binding of the first MBM to the TMEA reduces binding of the second MBM to the TMEA by no more than, and in some embodiments, less than, 50% (e.g., less than 40%, less than 30%, less than 20% or less than 20%) in a competition assay such as an ELISA assay, Biacore assay, FACS assay, or another competition assay in the art.

Preferably, TMEA 1 and/or TMEA 2 are human TMEAs. TMEA 1 and/or TMEA 2 may or may not be present on normal cells. In certain embodiments, TMEA 1 and/or TMEA 2 are preferentially expressed or upregulated on tumor cells as compared to normal cells. It is anticipated that any type of tumor and any type of TMEA may be targeted by the MBMs of the disclosure. Exemplary types of cancers that may be targeted include acute lymphoblastic leukemia, acute myelogenous leukemia, biliary cancer, B-cell leukemia, B-cell lymphoma, biliary cancer, bone cancer, brain cancer, breast cancer, triple-negative breast cancer, cervical cancer, Burkitt lymphoma, chronic lymphocytic leukemia, chronic myelogenous leukemia, colorectal cancer, endometrial cancer, esophageal cancer, gall bladder cancer, gastric cancer, gastrointestinal tract cancer, glioma, hairy cell leukemia, head and neck cancer, Hodgkin's lymphoma, liver cancer, lung cancer, medullary thyroid cancer, melanoma, multiple myeloma, ovarian cancer, non-Hodgkin's lymphoma, pancreatic cancer, prostate cancer, pulmonary tract cancer, renal cancer, sarcoma, skin cancer, testicular cancer, urothelial cancer, and other urinary bladder cancers. However, the skilled artisan will realize that TMEAs are known for virtually any type of cancer.

Exemplary TMEAs that can be targeted by MBMs of the disclosure include APRIL, FAP, BAFF, IL-1R, VEGF-A, VEGFR, CSF1R, ανβ3, and α5β1.

In some embodiments, a TMEA (e.g., TMEA 1 and/or TMEA 2) targeted by a MBM of the disclosure is APRIL. In other embodiments, a TMEA (e.g., TMEA 1 and/or TMEA 2) targeted by a MBM of the disclosure is FAP. In other embodiments, a TMEA (e.g., TMEA 1 and/or TMEA 2) targeted by a MBM of the disclosure is BAFF. In other embodiments, a TMEA (e.g., TMEA 1 and/or TMEA 2) targeted by a MBM of the disclosure is IL-1R. In other embodiments, a TMEA (e.g., TMEA 1 and/or TMEA 2) targeted by a MBM of the disclosure is VEGF-A. In other embodiments, a TMEA (e.g., TMEA 1 and/or TMEA 2) targeted by a MBM of the disclosure is VEGFR. In other embodiments, a TMEA (e.g., TMEA 1 and/or TMEA 2) targeted by a MBM of the disclosure is CSF1R. In other embodiments, a TMEA (e.g., TMEA 1 and/or TMEA 2) targeted by a MBM of the disclosure is ανβ3. In other embodiments, a TMEA (e.g., TMEA 1 and/or TMEA 2) targeted by a MBM of the disclosure is α5β1.

In some combinations of first and second MBMs, TMEA 1 and TMEA 2 are the same, and the TMEA is APRIL. In some combinations of first and second MBMs, TMEA 1 and TMEA 2 are the same, and the TMEA is FAP. In some combinations of first and second MBMs, TMEA 1 and TMEA 2 are the same, and the TMEA is BAFF. In some combinations of first and second MBMs, TMEA 1 and TMEA 2 are the same, and the TMEA is IL-1R. In some combinations of first and second MBMs, TMEA 1 and TMEA 2 are the same, and the TMEA is VEGF-A. In some combinations of first and second MBMs, TMEA 1 and TMEA 2 are the same, and the TMEA is VEGFR. In some combinations of first and second MBMs, TMEA 1 and TMEA 2 are the same, and the TMEA is CSF1R. In some combinations of first and second MBMs, TMEA 1 and TMEA 2 are the same, and the TMEA is ανβ3. In some combinations of first and second MBMs, TMEA 1 and TMEA 2 are the same, and the TMEA is α5β1. In some combinations of first and second MBMs, TMEA 1 and TMEA 2 are different. In some embodiments, TMEA 1 is APRIL and TMEA 2 is FAP (or vice versa). In some embodiments, TMEA 1 is APRIL and TMEA 2 is BAFF (or vice versa). In some embodiments, TMEA 1 is APRIL and TMEA 2 is IL-1R (or vice versa). In some embodiments, TMEA 1 is APRIL and TMEA 2 is VEGF-A (or vice versa). In some embodiments, TMEA 1 is APRIL and TMEA 2 is VEGFR (or vice versa). In some embodiments, TMEA 1 is APRIL and TMEA 2 is CSF1R (or vice versa). In some embodiments, TMEA 1 is APRIL and TMEA 2 is αΓβ3 (or vice versa). In some embodiments, TMEA 1 is APRIL and TMEA 2 is α5β1 (or vice versa). In some embodiments, TMEA 1 is FAP and TMEA 2 is BAFF (or vice versa). In some embodiments, TMEA 1 is FAP and TMEA 2 is IL-1R (or vice versa). In some embodiments, TMEA 1 is FAP and TMEA 2 is VEGF-A (or vice versa). In some embodiments, TMEA 1 is FAP and TMEA 2 is VEGFR (or vice versa). In some embodiments, TMEA 1 is FAP and TMEA 2 is CSF1R (or vice versa). In some embodiments, TMEA 1 is FAP and TMEA 2 is αΓβ3 (or vice versa). In some embodiments, TMEA 1 is FAP and TMEA 2 is α5β1 (or vice versa). In some embodiments, TMEA 1 is BAFF and TMEA 2 is IL-1R (or vice versa). In some embodiments, TMEA 1 is BAFF and TMEA 2 is VEGF-A (or vice versa). In some embodiments, TMEA 1 is BAFF and TMEA 2 is VEGFR (or vice versa). In some embodiments, TMEA 1 is BAFF and TMEA 2 is CSF1R (or vice versa). In some embodiments, TMEA 1 is BAFF and TMEA 2 is αΓβ3 (or vice versa). In some embodiments, TMEA 1 is BAFF and TMEA 2 is α5β1 (or vice versa). In some embodiments, TMEA 1 is IL-1R and TMEA 2 is VEGF-A (or vice versa). In some embodiments, TMEA 1 is IL-1R and TMEA 2 is VEGFR (or vice versa). In some embodiments, TMEA 1 is IL-1R and TMEA 2 is CSF1R (or vice versa). In some embodiments, TMEA 1 is IL-1R and TMEA 2 is αΓβ3 (or vice versa). In some embodiments, TMEA 1 is IL-1R and TMEA 2 is α5β1 (or vice versa). In some embodiments, TMEA 1 is VEGF-A and TMEA 2 is VEGFR (or vice versa). In some embodiments, TMEA 1 is VEGF-A and TMEA 2 is CSF1R (or vice versa). In some embodiments, TMEA 1 is VEGF-A and TMEA 2 is αΓβ3 (or vice versa). In some embodiments, TMEA 1 is VEGF-A and TMEA 2 is α5β1 (or vice versa). In some embodiments, TMEA 1 is VEGFR and TMEA 2 is CSF1R (or vice versa). In some embodiments, TMEA 1 is VEGFR and TMEA 2 is αΓβ3 (or vice versa). In some embodiments, TMEA 1 is VEGFR and TMEA 2 is α5β1 (or vice versa). In some embodiments, TMEA 1 is CSF1R and TMEA 2 is αΓβ3 (or vice versa). In some embodiments, TMEA 1 is CSF1R and TMEA 2 is α5β1 (or vice versa). In some embodiments, TMEA 1 is αΓβ3 and TMEA 2 is α5β1 (or vice versa).

A TMEA ABM can comprise, for example, a ligand- or an antibody-based moiety. Ligands and antibodies that bind to TMEAs are well-known in the art. In the case of antibody-based moieties, the anti-TMEA antibody or antigen-binding fragment can comprise, for example, the CDR sequences of an antibody set forth in Table 21. In some embodiments, the anti-TMEA antibody or antigen-binding domain thereof has the heavy and light chain variable region sequences of an antibody set forth in Table 21.

TABLE 21
Exemplary Anti-Tumor Microenvironment Antigen Antibodies

| Target | Examples of Antibody Name and/or Reference(s) and/or Source |
|---|---|
| APRIL | BION-1301; any anti-APRIL antibody described in WO/2017/091683, WO/2016/113368, |
| | WO/2010/100056, WO/2007/039489 |
| FAP | Ostermann et al., 2008, Clinical Cancer Research 14: 4584-4592 (FAP5), US Pat. |
| | Publication No. 2009/0304718; sibrotuzumab (see e.g., Hofheinz et al., 2003, Oncology |
| | Research and Treatment 26(1): 44-48); and Tran et al., 2013, J Exp Med 210(6): 1125- |
| | 1135. |
| BAFF | Any anti-BAFF antibody described in WO/2016/160476, WO/2016/039801, |
| | WO/2013/158577, WO/2006/025345 |
| IL-1R | ab106278 (Abcam); D1f3 (Abcam); ch2D8 (Creative Biolabs) |
| VEGF-A | Bevacizumab; any anti-VEGF-A antibody described in WO/2018/037001; |
| | WO/2018/037000; WO/2007/140534; WO/2015/166112 |
| VEGFR | Ramucirumab; any anti-VEFR antibody described in WO/2019/075270, WO/2017/117384, |
| | WO/2019/009879 |
| CSF1R | Emactuzumab; Cabiralizumab; SNDX-6352; any anti-CSF1R antibody described in |
| | WO/2011/140249, WO/2014/036357; WO/2013/169264; WO/2011/070024; |
| | WO/2015/200089 |
| αvβ3 | Vitaxin; Etaracizumab; LM609; any anti-αvβ3 antibody described in WO/1998/033919 |
| α5β1 | Volociximab; any anti-α5β1 antibody described in WO/2009/100110 |

### 7.9. TCR ABMs

Second MBMs of the disclosure (e.g., BBMs) can contain an ABM4 that specifically binds to a component of a TCR complex. The TCR is a disulfide-linked membrane-anchored heterodimeric protein normally consisting of the highly variable alpha (α) and beta (β) chains expressed as part of a complex with the invariant CD3 chain molecules. T cells expressing this receptor are referred to as α:β (or αβ) T cells, though a minority of T cells express an alternate receptor, formed by variable gamma (γ) and delta (δ) chains, referred as γδ T cells.

In an embodiment, second MBMs contain an ABM4 that specifically binds to CD3.

### 7.9.1. CD3 ABMs

The second MBMs (e.g., BBMs) can contain an ABM4 that specifically binds to CD3. The term “CD3” refers to the cluster of differentiation 3 co-receptor (or co-receptor complex, or polypeptide chain of the co-receptor complex) of the T cell receptor. The amino acid sequence of the polypeptide chains of human CD3 are provided in NCBI Accession P04234, P07766 and P09693. CD3 proteins can also include variants. CD3 proteins can also include fragments. CD3 proteins also include post-translational modifications of the CD3 amino acid sequences. Post-translational modifications include, but are not limited to, N- and O-linked glycosylation.

In some embodiments, a second MBM (e.g., BBM) can comprise an ABM4 which is an anti-CD3 antibody (e.g., as described in US 2016/0355600, WO 2014/110601, and WO 2014/145806) or an antigen-binding domain thereof. Exemplary anti-CD3 VH, VL, and scFV sequences that can be used in MBMs (e.g., BBMs) are provided in Table 22A.

TABLE 22A
CD3 Binders-Variable domain sequences

| Binding | | | SEQ |
|---|---|---|---|
| Domain | Chain | Sequence | ID NO: |
| CD3-1 | VH | QVQLQQSGAELARPGASVKMSCKASGYTFTRYTMHWWKQRPGQ | 136 |
| | | GLEWIGYINPSRGYTNYNQKFKDKATLTTDKSSSTAYMQLSSLTSE | |
| | | DSAVYYCARYYDDHYCLDYWGQGTTLTVSS | |
| | VL | QIVLTQSPAIMSASPGEKVTMTCSASSSVSYMNWYQQKSGTSPKR | 137 |
| | | WIYDTSKLASGVPAHFRGSGSGTSYSLTISGMEAEDAATYYCQQW | |
| | | SSNPFTFGSGTKLEIN | |
| CD3-2 | VH | EVQLVESGGGLVQPKGSLKLSCAASGFTFNTYAMNWVRQAPGKG | 138 |
| | | LEWVARIRSKYNNYATYYADSVKDRFTISRDDSQSILYLQMNNLKT | |
| | | EDTAMYYCVRHGNFGNSYVSWFAYWGQGTLVTVSA | |
| | VL | QAVVTQESALTTSPGETVTLTCRSSTGAVTTSNYANWVQEKPDHL | 139 |
| | | FTGLIGGTNKRAPGVPARFSGSLIGDKAALTITGAQTEDEAIYFCAL | |
| | | WYSN LVWFGGGTKLTVL | |
| CD3-3 | VH | QVQLQQSGAELARPGASVKMSCKASGYTFTSYTMHWVKQRPGQ | 140 |
| | | GLEWIGYINPSSGYTKYNQKFKDKATLTADKSSSTAYMQLSSLTSE | |
| | | DSAVYYCARWQDYDVYFDYWGQGTTLTVSS | |
| | VL | QIVLSQSPAILSASPGEKVTMTCRASSSVSYMHWYQQKPGSSPKP | 141 |
| | | WIYATSNLASGVPARFSGSGSGTSYSLTISRVEAEDAATYYCQQW | |
| | | SSNPPTFGGGTKLETK | |
| CD3-4 | VH | QVQLQQSGAELARPGASVKMSCKASGYTFTRYTMHWVKQRPGQ | 136 |
| | | GLEWIGYINPSRGYTNYNQKFKDKATLTTDKSSSTAYMQLSSLTSE | |
| | | DSAVYYCARYYDDHYCLDYWGQGTTLTVSS | |
| | VL | QIVLTQSPAIMSASPGEKVTMTCRASSSVSYMNWYQQKSGTSPKR | 142 |
| | | WIYDTSKVASGVPYRFSGSGSGTSYSLTISSMEAEDAATYYCQQW | |
| | | SSNPLTFGSGTKLEIN | |
| CD3-5 | VH | QVQLVQSGGGVVQPGRSLRLSCKASGYTFTRYTMHWVRQAPGK | 143 |
| | | GLEWIGYINPSRGYTNYNQKVKDRFTISRDNSKNTAFLQMDSLRPE | |
| | | DTGVYFCARYYDDHYCLDYWGQGTPVTVSS | |
| | VL | DIQMTQSPSSLSASVGDRVTITCSASSSVSYMNWYQQTPGKAPKR | 144 |
| | | WIYDTSKLASGVPSRFSGSGSGTDYTFTISSLQPEDIATYYCQQWS | |
| | | SNPFTFGQGTKLQIT | |
| CD3-6 | VH | QVQLVESGGGVVQPGRSLRLSCAASGFKFSGYGMHWVRQAPGK | 145 |
| | | GLEWVAVIWYDGSKKYYVDSVKGRFTISRDNSKNTLYLQMNSLRA | |
| | | EDTAVYYCARQMGYWHFDLWGRGTLVTVSS | |
| | VL | EIVLTQSPATLSLSPGERATLSCRASQSVSSYLAWYQQKPGQAPR | 146 |
| | | LLIYDASNRATGIPARFSGSGSGTDFTLTISSLEPEDFAVYYCQQRS | |
| | | NWPPLTFGGGTKVEIK | |
| CD3-7 | VH | EVQLVESGGGLVQPGGSLRLSCAASGFTFSTYAMNWVRQAPGKG | 147 |
| | | LEWVGRIRSKYNNYATYYADSVKDRFISRDDSKNSLYLQMNSLKTE | |
| | | DTAVYYCVRHGNFGNSYVSWFAYWGQGTLVTVSS | |
| | VL | QAVVTQEPSLTVSPGGTVTLTCRSSTGAVTTSNYANWVQQKPGQ | 148 |
| | | APRGLIGGTNKRAPWTPARFSGSLLGGKAALIGAQAEDEADYYCA | |
| | | LWYSNLWVFGGGTKLTVL | |
| CD3-8 | VH | DIKLQQSGAELARPGASVKMSCKTSGYTFTRYTMHWVKQRPGQG | 149 |
| | | LEWIGYINPSRGYTNYNQKFKDKATLTTDKSSSTAYMQLSSLTSED | |
| | | SAVYYCARYYDDHYCLDYWGQGTTLTVSS | |
| | VL | DIQLTQSPAIMSASPGEKVTMTCRASSSVSYMNWYQQKSGTSPKR | 150 |
| | | WIYDTSKVASGVPYRFSGSGSGTSYSLISSMEAEDAATYYCQQWS | |
| | | SNPLTFGAGTKLELK | |
| CD3-9 | VH | EVQLVESGGGLVQPGGSLRLSCAASGFTFNTYAMNWVRQAPGKG | 151 |
| | | LEWVARIRSKYNNYATYYADSVKDRFISRDDSKNSLYLQMNSLKTE | |
| | | DTAVYYCVRHGNFGNSYVSWFAYWGQGTLVTVSS | |
| | VL | QAVVTQEPSLTVSPGGTVTLTCRSSTGAVTTSNYANWVQQKPGQ | 148 |
| | | APRGLIGGTNKRAPWTPARFSGSLLGGKAALIGAQAEDEADYYCA | |
| | | LWYSNLWVFGGGTKLTVL | |
| CD3-10 | VH | EVKLLESGGGLVQPKGSLKLSCAASGFTFNTYAMNWVRQAPGKG | 152 |
| | | LEWVARIRSKYNNYATYYADSVKDRFTISRDDSQSILYLQMNNLKT | |
| | | EDTAMYYCVRHGNFGNSYVSWFAYWGQGTLVTVSA | |
| | VL | QAVVTQESALTTSPGETVTLTCRSSTGAVTTSNYANWVQEKPDHL | 139 |
| | | FTGLIGGTNKRAPGVPARFSGSLIGDKAALTITGAQTEDEAIYFCAL | |
| | | WYSN LWVFGGGTKLTVL | |
| CD3-11 | VH | EVQLVESGGGLVQPGGSLKLSCAASGFTFNSYAMNWVRQAPGKG | 153 |
| | | LEWVARIRSKYNNYATYYADSVKGRFTISRDDSKNTAYLQMNNLKT | |
| | | EDTAVYYCVRHGNFGNSYVSWWAYWGQGTLVTVSS | |
| | VL | QTVVTQEPSLTVSPGGTVTLTCGSSTGAVTSGNYPNWVQQKPGQ | 154 |
| | | APRGLIGGTKFLAPGTPQRFSGSLLGGKAALTLSGVQPEDEAEYYC | |
| | | VLWYSN RWVFGGGTKLTVL | |
| CD3-12 | VH | EVQLVESGGGLVQPGGSLKLSCAASGFTFNKYAMNWVRQAPGKG | 155 |
| | | LEWVARIRSKYNNYATYYADSVKDRFTISRDDSKNTAYLQMNNLKT | |
| | | EDTAVYYCVRHGNFGNSYISYWAYWGQGTLVTVSS | |
| | VL | QTVVTQEPSLTVSPGGTVTLTCGSSTGAVTSGNYPNWVQQKPGQ | 156 |
| | | APRGLIGGTKFLAPGTPARFSGSLLGGKAALTLSGVQPEDEAEYYC | |
| | | VLWYSNRWVFGGGTKLTVL | |
| | scFv | EVQLVESGGGLVQPGGSLKLSCAASGFTFNKYAMNWVRQAPGKG | 157 |
| | | LEWVARIRSKYNNYATYYADSVKDRFTISRDDSKNTAYLQMNNLKT | |
| | | EDTAVYYCVRHGNFGNSYISYWAYWGQGTLVTVSSGGGGSGGG | |
| | | GSGGGGSGGGGSQTVVTQEPSLTVSPGGTVTLTCGSSTGAVTSG | |
| | | NYPNWVQQKPGQAPRGLIGGTKFLAPGTPARFSGSLLGGKAALTL | |
| | | SGVQPEDEAEYYCVLWYSNRWVFGGGTKLTVL | |
| CD3-13 | VH | QVQLVQSGAEVKKPGASVKVSCKASGYTFTRYTMHWVRQAPGQ | 158 |
| | | GLEWMGYINPSRGYTNYNQKFKDRVTMTTDTSISTAYMELSRLRS | |
| | | DDTAVYYCARYYDDHYCLDYWGQGTLVTVSS | |
| | VL | EIVLTQSPATLSLSPGERATLSCSASSSVSYMNWYQQKPGQAPRL | 159 |
| | | LIYDTSKLASGVPAHFRGSGSGTDFTLTISSLEPEDFAVYYCQQWS | |
| | | SNPFTFGQGTKVEIK | |
| CD3-14 | VH | EVQLVESGGGLVQPGGSLRLSCAASGFTFSTYAMNWVRQAPGKG | 160 |
| | | LEWVSRIRSKYNNYATYYADSVKDRFTISRDDSKNTLYLQMNSLRA | |
| | | EDTAVYYCARHGNFGNSYVSWFAYWGQGTMVTVSS | |
| | VL | QAVVTQEPSLTVSPGGTVTLTCRSSTGAVTTSNYANWVQQKPGQ | 161 |
| | | APRGLIGGTNKRAPGTPARFSGSLLGGKAALTLSGVQPEDEAEYY | |
| | | CALWYSNLWVFGGGTKLTVL | |
| CD3-15 | VH | EVQLVESGGGLVQPGGSLRLSCAASGFTFNTYAMNWVRQAPGKG | 162 |
| | | LEWVGRIRSKYNNYATYYADSVKDRFTISRDDSKNSLYLQMNSLKT | |
| | | EDTAVYYCVRHGNFGNSYVSWFAYWGQGTLVTVSS | |
| | VL | QAVVTQEPSLTVSPGGTVTLTCRSSTGAVTTSNYANWVQEKPGQ | 163 |
| | | APRGLIGGTNKRAPWTPARFSGSLLGGKAALTITGAQAEDEADYY | |
| | | CALWYSNLWVFGGGTKLTVL | |
| CD3-16 | VH | EVQLVESGGGLVQPGGSLRLSCAASGFTFNTYAMNWVRQAPGKG | 164 |
| | | LEWVGRIRSKYNNYATYYADSVKGRFTISRDDSKNTLYLQMNSLRA | |
| | | EDTAVYYCVRHGNFGNSYVSWFAYWGQGTLVTVSS | |
| | VL | QAVVTQEPSLTVSPGGTVTLTCGSSTGAVTTSNYANWVQQKPGQ | 165 |
| | | APRGLIGGTNKRAPGVPARFSGSLLGGKAALTLSGAQPEDEAEYY | |
| | | CALWYSNLWVFGGGTKLTVL | |
| CD3-17 | VH | EVQLVESGGGLVQPGGSLRLSCAASGFTFSTYAMNWVRQAPGKG | 166 |
| | | LEWVGRIRSKYNNYATYYADSVKGRFTISRDDSKNTLYLQMNSLRA | |
| | | EDTAVYYCVRHGNFGDSYVSWFAYWGQGTLVTVSS | |
| | VL | QAVVTQEPSLTVSPGGTVTLTCGSSTGAVTTSNYANWVQQKPGK | 167 |
| | | SPRGLIGGTNKRAPGVPARFSGSLLGGKAALTISGAQPEDEADYYC | |
| | | ALWYSNHWVFGGGTKLTVL | |
| CD3-18 | VH | QVQLVQSGGGVVQPGRSLRLSCKASGYTFTRYTMHWVRQAPGK | 143 |
| | | GLEWIGYINPSRGYTNYNQKVKDRFTISRDNSKNTAFLQMDSLRPE | |
| | | DTGVYFCARYYDDHYCLDYWGQGTPVTVSS | |
| | VL | DIQMTQSPSSLSASVGDRVTITCSASSSVSYMNWYQQTPGKAPKR | 168 |
| | | WIYDTSKLASGVPSRFSGSGSGTDYTFTISSLQPEDIATYYCQQWS | |
| | | SNPFTFGQGT | |
| CD3-19 | VH | QVQLVQSGGGVVQPGRSLRLSCKASGYTFTRYTMHWVRQAPGK | 169 |
| | | GLEWIGYINPSRGYTNYNQKVKDRFTISRDNSKNTAFLQMDSLRPE | |
| | | DTGVYFCARYYDDHYSLDYWGQGTPVTVSS | |
| | VL | DIQMTQSPSSLSASVGDRVTITCSASSSVSYMNWYQQTPGKAPKR | 168 |
| | | WIYDTSKLASGVPSRFSGSGSGTDYTFTISSLQPEDIATYYCQQWS | |
| | | SNPFTFGQGT | |
| CD3-20 | VH | EVQLQQSGPELVKPGASMKISCKASGYSFTGYTMNWVKQSHGKN | 170 |
| | | LEWMGLINPYKGVSTYNQKFKDKATLTVDKSSSTAYMELLSLTSED | |
| | | SAVYYCARSGYYGDSDWYFDVWGQGTTLTVFS | |
| | VL | DIQMTQTTSSLSASLGDRVTISCRASQDIRNYLNWYQQKPDGTVKL | 171 |
| | | LIYYTSRLHSGVPSKFSGSGSGTDYSLTISNLEQEDIATYFCQQGNT | |
| | | LPWTFAGGTKLEIK | |
| CD3-21 | VH | EVQLVESGGGLVQPGGSLKLSCAASGFTFNTYAMNWVRQASGKG | 172 |
| | | LEWVGRIRSKYNNYATYYADSVKDRFTISRDDSKSTLYLQMNSLKT | |
| | | EDTAVYYCVRHGNFGNSYVSWFAYWGQGTLVTVSS | |
| | VL | QAVVTQEPSLTVSPGGTVTLTCRSSTGAVTTSNYANWVQQKPGQ | 173 |
| | | APRGLIGGTNKRAPWTPARFSGSLLGDKAALTLSGAQPEDEAEYF | |
| | | CALWYSNLWVFGGGTKLTVL | |
| | scFv | EVQLVESGGGLVQPGGSLKLSCAASGFTFNTYAMNWVRQASGKG | 174 |
| | | LEWVGRIRSKYNNYATYYADSVKDRFTISRDDSKSTLYLQMNSLKT | |
| | | EDTAVYYCVRHGNFGNSYVSWFAYWGQGTLVTVSSGGGGSGGG | |
| | | GSGGGGSGGGGSQAVVTQEPSLTVSPGGTVTLTCRSSTGAVTTS | |
| | | NYANWVQQKPGQAPRGLIGGTNKRAPWTPARFSGSLLGDKAALT | |
| | | LSGAQPEDEAEYFCALWYSNLWVFGGGTKLTVL | |
| CD3-22 | VH | EVQLVESGGGLVQPGGSLRLSCAASGFTFSTYAMNWVRQAPGKG | 166 |
| | | LEWVGRIRSKYNNYATYYADSVKGRFTISRDDSKNTLYLQMNSLRA | |
| | | EDTAVYYCVRHGNFGDSYVSWFAYWGQGTLVTVSS | |
| | VL | QAVVTQEPSLTVSPGGTVTLTCGSSTGAVTTSNYANWVQQKPGK | 167 |
| | | SPRGLIGGTNKRAPGVPARFSGSLLGGKAALTISGAQPEDEADYYC | |
| | | ALWYSNHWVFGGGTKLTVL | |
| | scFv | EVQLVESGGGLVQPGGSLRLSCAASGFTFSTYAMNWVRQAPGKG | 175 |
| | | LEWVGRIRSKYNNYATYYADSVKGRFTISRDDSKNTLYLQMNSLRA | |
| | | EDTAVYYCVRHGNFGDSYVSWFAYWGQGTLVTVSSGKPGSGKP | |
| | | GSGKPGSGKPGSQAVVTQEPSLTVSPGGTVTLTCGSSTGAVTTSN | |
| | | YANWVQQKPGKSPRGLIGGTNKRAPGVPARFSGSLLGGKAALTIS | |
| | | GAQPEDEADYYCALWYSNHWVFGGGTKLTVL | |
| CD3-23 | VH | EVQLVESGGGLVQPGGSLRLSCAASGFTFSTYAMNWVRQAPGKG | 176 |
| | | LEWVGRIRSKANNYATYYADSVKGRFTISRDDSKNTLYLQMNSLRA | |
| | | EDTAVYYCVRHGNFGDSYVSWFAYWGQGTLVTVSS | |
| | VL | QAVVTQEPSLTVSPGGTVTLTCGSSTGAVTTSNYANWVQQKPGK | 167 |
| | | SPRGLIGGTNKRAPGVPARFSGSLLGGKAALTISGAQPEDEADYYC | |
| | | ALWYSNHWWVFGGGTKLTVL | |
| | scFv | EVQLVESGGGLVQPGGSLRLSCAASGFTFSTYAMNWVRQAPGKG | 177 |
| | | LEWVGRIRSKANNYATYYADSVKGRFTISRDDSKNTLYLQMNSLRA | |
| | | EDTAVYYCVRHGNFGDSYVSWFAYWGQGTLVTVSSGKPGSGKP | |
| | | GSGKPGSGKPGSQAVVTQEPSLTVSPGGTVTLTCGSSTGAVTTSN | |
| | | YANWVQQKPGKSPRGLIGGTNKRAPGVPARFSGSLLGGKAALTIS | |
| | | GAQPEDEADYYCALWYSNHWVFGGGTKLTVL | |
| CD3-24 | VH | EVQLVESGGGLVQPGGSLRLSCAASGFTFSTYAMNWVRQAPGKG | 178 |
| | | LEWVGRIRSKYNNYATYYADSVKGRFTISRDDSKNTLYLQMNSLRA | |
| | | EDTAVYYCVRHGNFGDEYVSWFAYWGQGTLVTVSS | |
| | VL | QAVVTQEPSLTVSPGGTVTLTCGSSTGAVTTSNYANWVQQKPGK | 167 |
| | | SPRGLIGGTNKRAPGVPARFSGSLLGGKAALTISGAQPEDEADYYC | |
| | | ALWYSNHWWVFGGGTKLTVL | |
| | scFv | EVQLVESGGGLVQPGGSLRLSCAASGFTFSTYAMNWVRQAPGKG | 179 |
| | | LEWVGRIRSKYNNYATYYADSVKGRFTISRDDSKNTLYLQMNSLRA | |
| | | EDTAVYYCVRHGNFGDEYVSWFAYWGQGTLVTVSSGKPGSGKP | |
| | | GSGKPGSGKPGSQAVVTQEPSLTVSPGGTVTLTCGSSTGAVTTSN | |
| | | YANWVQQKPGKSPRGLIGGTNKRAPGVPARFSGSLLGGKAALTIS | |
| | | GAQPEDEADYYCALWYSNHWVFGGGTKLTVL | |
| CD3-25 | VH | EVQLVESGGGLVQPGGSLRLSCAASGFTFSTYAMNWVRQAPGKG | 180 |
| | | LEWVGRIRSKYNNYATYYADSVKGRFTISRDDSKNTLYLQMNSLRA | |
| | | EDTAVYYCVRHGNFGDPYVSWFAYWGQGTLVTVSS | |
| | VL | QAVVTQEPSLTVSPGGTVTLTCGSSTGAVTTSNYANWVQQKPGK | 167 |
| | | SPRGLIGGTNKRAPGVPARFSGSLLGGKAALTISGAQPEDEADYYC | |
| | | ALWYSNHWWVFGGGTKLTVL | |
| | scFv | EVQLVESGGGLVQPGGSLRLSCAASGFTFSTYAMNWVRQAPGKG | 181 |
| | | LEWVGRIRSKYNNYATYYADSVKGRFTISRDDSKNTLYLQMNSLRA | |
| | | EDTAVYYCVRHGNFGDPYVSWFAYWGQGTLVTVSSGKPGSGKP | |
| | | GSGKPGSGKPGSQAVVTQEPSLTVSPGGTVTLTCGSSTGAVTTSN | |
| | | YANWVQQKPGKSPRGLIGGTNKRAPGVPARFSGSLLGGKAALTIS | |
| | | GAQPEDEADYYCALWYSNHWVFGGGTKLTVL | |
| CD3-26 | VH | EVQLVESGGGLVQPGGSLRLSCAASGFTFSTYAMNWVRQAPGKG | 182 |
| | | LEWVGRIRSKYNNYATYYADSVKGRFTISRDDSKNTLYLQMNSLRA | |
| | | EDTAVYYCVRHGNFGDSYVSWFDYWGQGTLVTVSS | |
| | VL | QAVVTQEPSLTVSPGGTVTLTCGSSTGAVTTSNYANWVQQKPGK | 167 |
| | | SPRGLIGGTNKRAPGVPARFSGSLLGGKAALTISGAQPEDEADYYC | |
| | | ALWYSNHWVFGGGTKLTVL | |
| | scFv | EVQLVESGGGLVQPGGSLRLSCAASGFTFSTYAMNWVRQAPGKG | 183 |
| | | LEWVGRIRSKYNNYATYYADSVKGRFTISRDDSKNTLYLQMNSLRA | |
| | | EDTAVYYCVRHGNFGDSYVSWFDYWGQGTLVTVSSGKPGSGKP | |
| | | GSGKPGSGKPGSQAVVTQEPSLTVSPGGTVTLTCGSSTGAVTTSN | |
| | | YANWVQQKPGKSPRGLIGGTNKRAPGVPARFSGSLLGGKAALTIS | |
| | | GAQPEDEADYYCALWYSNHWVFGGGTKLTVL | |
| CD3-27 | VH | EVQLVESGGGLVQPGGSLRLSCAASGFTFSTYAMSWVRQAPGKG | 184 |
| | | LEWVGRIRSKYNNYATYYADSVKGRFTISRDDSKNTLYLQMNSLRA | |
| | | EDTAVYYCVRHGNFGDSYVSWFAYWGQGTLVTVSS | |
| | VL | QAVVTQEPSLTVSPGGTVTLTCGSSTGAVTTSNYANWVQQKPGK | 167 |
| | | SPRGLIGGTNKRAPGVPARFSGSLLGGKAALTISGAQPEDEADYYC | |
| | | ALWYSNHWVFGGGTKLTVL | |
| | scFv | EVQLVESGGGLVQPGGSLRLSCAASGFTFSTYAMSWVRQAPGKG | 185 |
| | | LEWVGRIRSKYNNYATYYADSVKGRFTISRDDSKNTLYLQMNSLRA | |
| | | EDTAVYYCVRHGNFGDSYVSWFAYWGQGTLVTVSSGKPGSGKP | |
| | | GSGKPGSGKPGSQAVVTQEPSLTVSPGGTVTLTCGSSTGAVTTSN | |
| | | YANWVQQKPGKSPRGLIGGTNKRAPGVPARFSGSLLGGKAALTIS | |
| | | GAQPEDEADYYCALWYSNHWVFGGGTKLTVL | |
| CD3-28 | VH | EVQLVESGGGLVQPGGSLRLSCAASGFTFNTYAMNWVRQAPGKG | 164 |
| | | LEWVGRIRSKYNNYATYYADSVKGRFTISRDDSKNTLYLQMNSLRA | |
| | | EDTAVYYCVRHGNFGNSYVSWFAYWGQGTLVTVSS | |
| | VL | QAVVTQEPSLTVSPGGTVTLTCGSSTGAVTTSNYANWVQQKPGQ | 165 |
| | | APRGLIGGTNKRAPGVPARFSGSLLGGKAALTLSGAQPEDEAEYY | |
| | | CALWYSNLWVFGGGTKLTVL | |
| | scFv | EVQLVESGGGLVQPGGSLRLSCAASGFTFNTYAMNWVRQAPGKG | 186 |
| | | LEWVGRIRSKYNNYATYYADSVKGRFTISRDDSKNTLYLQMNSLRA | |
| | | EDTAVYYCVRHGNFGNSYVSWFAYWGQGTLVTVSSGGGGSGGG | |
| | | GSGGGGSQAVVTQEPSLTVSPGGTVTLTCGSSTGAVTTSNYANW | |
| | | VQQKPGQAPRGLIGGTNKRAPGVPARFSGSLLGGKAALTLSGAQP | |
| | | EDEAEYYCALWYSNLWVFGGGTKLTVLGSHHHHHH | |
| CD3-129 | VH | EVQLVESGGGLVQPGGSLKLSCAASGFTFNTYAMNWVRQASGKG | 187 |
| | | LEWWGRIRSKYNNYATYYADSVKDRFTISRDDSKNTAYLQMNSLKT | |
| | | EDTAVYYCVRHGNFGNSYVSWFAHWGQGTLVTVSS | |
| | VL | QAVVTQEPSLTVSPGGTVTLTCGSSTGAVTSSNYANWVQQKPGQ | 188 |
| | | APRGLIGGTNKRAPWTPARFSGSLLGGKAALTLSGAQPEDEAEYY | |
| | | CALWYSNLWWFGGGTKLTVL | |
| | scFv | EVQLVESGGGLVQPGGSLKLSCAASGFTFNTYAMNWVRQASGKG | 189 |
| | | LEWVGRIRSKYNNYATYYADSVKDRFTISRDDSKNTAYLQMNSLKT | |
| | | EDTAVYYCVRHGNFGNSYVSWFAHWGQGTLVTVSSGGGGSGGG | |
| | | GSGGGGSGGGGSQAVVTQEPSLTVSPGGTVTLTCGSSTGAVTSS | |
| | | NYANWVQQKPGQAPRGLIGGTNKRAPWTPARFSGSLLGGKAALT | |
| | | LSGAQPEDEAEYYCALWYSNLWVFGGGTKLTVL | |
| CD3-130 | VH | EVQLVESGGGLVQPGGSLKLSCAASGFTFNTYAMNWVRQASGKG | 190 |
| | | LEWVGRIRSKYNNYATYYADSVKDRFTISRDDSKSTAYLQMNSLKT | |
| | | EDTAVYYCVRHGNFGNSYVSWFAYWGQGTLVTVSS | |
| | VL | QAVVTQEPSLTVSPGGTVTLTCRSSTGAVTTSNYANWVQQKPGQ | 173 |
| | | APRGLIGGTNKRAPWTPARFSGSLLGDKAALTLSGAQPEDEAEYF | |
| | | CALWYSNLWWFGGGTKLTVL | |
| | scFv | EVQLVESGGGLVQPGGSLKLSCAASGFTFNTYAMNWVRQASGKG | 191 |
| | | LEWVGRIRSKYNNYATYYADSVKDRFTISRDDSKSTAYLQMNSLKT | |
| | | EDTAVYYCVRHGNFGNSYVSWFAYWGQGTLVTVSSGGGGSGGG | |
| | | GSGGGGSGGGGSQAVVTQEPSLTVSPGGTVTLTCRSSTGAVTTS | |
| | | NYANWVQQKPGQAPRGLIGGTNKRAPWTPARFSGSLLGDKAALT | |
| | | LSGAQPEDEAEYFCALWYSNLWVFGGGTKLTVL | |

CDR sequences for a number of CD3 binders as defined by the Kabat numbering scheme (Kabat et al, 1991, Sequences of Proteins of Immunological Interest, 5^{th} Ed. Public Health Service, National Institutes of Health, Bethesda, Md.), Chothia numbering scheme (Al-Lazikani et al., 1997, J. Mol. Biol 273:927-948), and a combination of Kabat and Chothia numbering are provided in Tables 22B-22D, respectively.

TABLE 22B
CD3 Binders-CDR sequences according to Kabat numbering scheme

| Binding | | | SEQ ID | | SEQ ID | | SEQ ID |
|---|---|---|---|---|---|---|---|
| Domain | Chain | CDR1 | NO: | CDR2 | NO: | CDR3 | NO: |
| CD3-1 | VH | RYTMH | 192 | YINPSRGYTNYNQKF | 212 | YYDDHYCLDY | 236 |
| | | | | KD | | | |
| | VL | SASSSVSYM | 193 | DTSKLAS | 213 | QQWSSNPFT | 237 |
| | | N | | | | | |
| CD3-2 | VH | TYAMN | 194 | RIRSKYNNYATYYAD | 214 | HGNFGNSYVS | 238 |
| | | | | SVKD | | WFAY | |
| | VL | RSSTGAVTT | 195 | GTNKRAP | 215 | ALWYSNLWV | 239 |
| | | SNYAN | | | | | |
| CD3-3 | VH | SYTMH | 196 | YINPSSGYTKYNQKF | 216 | WQDYDVYFDY | 240 |
| | | | | KD | | | |
| | VL | RASSSVSYM | 197 | ATSNLAS | 217 | QQWSSNPPT | 241 |
| | | H | | | | | |
| CD3-4 | VH | RYTMH | 192 | YINPSRGYTNYNQKF | 212 | YYDDHYCLDY | 236 |
| | | | | KD | | | |
| | VL | RASSSVSYM | 198 | DTSKVAS | 218 | QQWSSNPLT | 242 |
| | | N | | | | | |
| CD3-5 | VH | RYTMH | 192 | YINPSRGYTNYNQKV | 219 | YYDDHYCLDY | 236 |
| | | | | KD | | | |
| | VL | SASSSVSYM | 193 | DTSKLAS | 213 | QQWSSNPFT | 237 |
| | | N | | | | | |
| CD3-6 | VH | GYGMH | 199 | VIWYDGSKKYYVDSV | 220 | QMGYWHFDL | 243 |
| | | | | KG | | | |
| | VL | RASQSVSSY | 200 | DASNRAT | 221 | QQRSNWPPLT | 244 |
| | | LA | | | | | |
| CD3-7 | VH | TYAMN | 194 | RIRSKYNNYATYYAD | 222 | VRHGNFGNSYV | 245 |
| | | | | | | SWFAY | |
| | VL | RSSTGAVTT | 195 | GTNKRAP | 215 | ALWYSNLWV | 239 |
| | | SNYAN | | | | | |
| CD3-8 | VH | RYTMH | 192 | YINPSRGYTNYNQKF | 212 | YYDDHYCLDY | 236 |
| | | | | KD | | | |
| | VL | RASSSVSYM | 198 | DTSKVAS | 218 | QQWSSNPLT | 242 |
| | | N | | | | | |
| CD3-9 | VH | TYAMN | 194 | RIRSKYNNYATYYAD | 222 | VRHGNFGNSYV | 245 |
| | | | | | | SWFAY | |
| | VL | RSSTGAVTT | 195 | GTNKRAP | 215 | ALWYSNLWV | 239 |
| | | SNYAN | | | | | |
| CD3-10 | VH | TYAMN | 194 | RIRSKYNNYATYYAD | 214 | HGNFGNSYVS | 238 |
| | | | | SVKD | | WFAY | |
| | VL | RSSTGAVTT | 195 | GTNKRAP | 215 | ALWYSNLWV | 239 |
| | | SNYAN | | | | | |
| CD3-11 | VH | SYAMN | 201 | RIRSKYNNYATYYAD | 223 | HGNFGNSYVS | 246 |
| | | | | SVKG | | WWAY | |
| | VL | GSSTGAVTS | 202 | GTKFLAP | 224 | VLWYSNRWV | 247 |
| | | GNYPN | | | | | |
| CD3-12 | VH | KYAMN | 203 | RIRSKYNNYATYYAD | 214 | HGNFGNSYISY | 248 |
| | | | | SVKD | | WAY | |
| | VL | GSSTGAVTS | 202 | GTKFLAP | 224 | VLWYSNRWW | 247 |
| | | GNYPN | | | | | |
| CD3-13 | VH | RYTMH | 192 | YINPSRGYTNYNQKF | 212 | YYDDHYCLDY | 236 |
| | | | | KD | | | |
| | VL | SASSSVSYM | 193 | DTSKLAS | 213 | QQWSSNPFT | 237 |
| | | N | | | | | |
| CD3-14 | VH | TYAMN | 194 | RIRSKYNNYATYYAD | 214 | HGNFGNSYVS | 238 |
| | | | | SVKD | | WFAY | |
| | VL | RSSTGAVTT | 195 | GTNKRAP | 215 | ALWYSNLWV | 239 |
| | | SNYAN | | | | | |
| CD3-15 | VH | TYAMN | 194 | RIRSKYNNYATYYAD | 214 | HGNFGNSYVS | 238 |
| | | | | SVKD | | WFAY | |
| | VL | RSSTGAVTT | 195 | GTNKRAP | 215 | ALWYSNLWV | 239 |
| | | SNYAN | | | | | |
| CD3-16 | VH | TYAMN | 194 | RIRSKYNNYATYYAD | 223 | HGNFGNSYVS | 238 |
| | | | | SVKG | | WFAY | |
| | VL | GSSTGAVTT | 204 | GTNKRAP | 215 | ALWYSNLWV | 239 |
| | | SNYAN | | | | | |
| CD3-17 | VH | TYAMN | 194 | RIRSKYNNYATYYAD | 223 | HGNFGDSYVS | 249 |
| | | | | SVKG | | WFAY | |
| | VL | GSSTGAVTT | 204 | GTNKRAP | 215 | ALWYSNHWV | 250 |
| | | SNYAN | | | | | |
| CD3-18 | VH | RYTMH | 192 | YINPSRGYTNYNQKV | 219 | YYDDHYCLDY | 236 |
| | | | | KD | | | |
| | VL | SASSSVSYM | 193 | DTSKLAS | 213 | QQWSSNPFT | 237 |
| | | N | | | | | |
| CD3-19 | VH | RYTMH | 192 | YINPSRGYTNYNQKV | 219 | YYDDHYSLDY | 251 |
| | | | | KD | | | |
| | VL | SASSSVSYM | 193 | DTSKLAS | 213 | QQWSSNPFT | 237 |
| | | N | | | | | |
| CD3-20 | VH | GYTMN | 205 | LINPYKGVSTYNQKF | 225 | SGYYGDSDWY | 252 |
| | | | | KD | | FDV | |
| | VL | RASQDIRNY | 206 | YTSRLH | 226 | QQGNTLPWT | 253 |
| | | LN | | | | | |
| CD3-21 | VH | TYAMN | 194 | RIRSKYNNYATYYAD | 214 | HGNFGNSYVS | 238 |
| | | | | SVKD | | WFAY | |
| | VL | RSSTGAVTT | 195 | GTNKRAP | 215 | ALWYSNLWV | 239 |
| | | SNYAN | | | | | |
| CD3-22 | VH | TYAMN | 194 | RIRSKYNNYATYYA | 223 | HGNFGDSYVS | 249 |
| | | | | DSVKG | | WFAY | |
| | VL | GSSTGAVT | 204 | GTNKRAP | 215 | ALWYSNHWV | 250 |
| | | TSNYAN | | | | | |
| CD3-23 | VH | TYAMN | 194 | RIRSKANNYATYYA | 227 | HGNFGDSYVS | 249 |
| | | | | DSVKG | | WFAY | |
| | VL | GSSTGAVT | 204 | GTNKRAP | 215 | ALWYSNHWV | 250 |
| | | TSNYAN | | | | | |
| CD3-24 | VH | TYAMN | 194 | RIRSKYNNYATYYA | 223 | HGNFGDEYVS | 254 |
| | | | | DSVKG | | WFAY | |
| | VL | GSSTGAVT | 204 | GTNKRAP | 215 | ALWYSNHWV | 250 |
| | | TSNYAN | | | | | |
| CD3-25 | VH | TYAMN | 194 | RIRSKYNNYATYYA | 223 | HGNFGDPYVS | 255 |
| | | | | DSVKG | | WFAY | |
| | VL | GSSTGAVT | 204 | GTNKRAP | 215 | ALWYSNHWV | 250 |
| | | TSNYAN | | | | | |
| CD3-26 | VH | TYAMN | 194 | RIRSKYNNYATYYA | 223 | HGNFGDSYVS | 256 |
| | | | | DSVKG | | WFDY | |
| | VL | GSSTGAVT | 204 | GTNKRAP | 215 | ALWYSNHWV | 250 |
| | | TSNYAN | | | | | |
| CD3-27 | VH | TYAMS | 207 | RIRSKYNNYATYYA | 223 | HGNFGDSYVS | 249 |
| | | | | DSVKG | | WFAY | |
| | VL | GSSTGAVT | 204 | GTNKRAP | 215 | ALWYSNHWV | 250 |
| | | TSNYAN | | | | | |
| CD3-28 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-29 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-30 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-31 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-32 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-33 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-34 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-35 | VH | TYAMH | 208 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-36 | VH | TYAMS | 207 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-37 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-38 | VH | TYAMN | 194 | RIRSKANNYYATYY | 228 | HGNFGNSYVS | 238 |
| | | | | ADSVKG | | WFAY | |
| | VL | GSSTGAVT | 204 | GTNKRAP | 215 | ALWYSNLWV | 239 |
| | | TSNYAN | | | | | |
| CD3-39 | VH | TYAMN | 194 | RIRSKANSYATYYA | 229 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-40 | VH | TYAMN | 194 | RIRSKYNNYATAYA | 230 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-41 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-42 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-43 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-44 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-45 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-46 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-47 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-48 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-49 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-50 | VH | TYAMN | 194 | RIRSKYNNYATYYA | 223 | HGNFGDSYVS | 249 |
| | | | | DSVKG | | WFAY | |
| | VL | GSSTGAVT | 204 | GTNKRAP | 215 | ALWYSNLWV | 239 |
| | | TSNYAN | | | | | |
| CD3-51 | VH | TYAMN | 194 | RIRSKYNNYATYYA | 223 | HGNFGQSYVS | 257 |
| | | | | DSVKG | | WFAY | |
| | VL | GSSTGAVT | 204 | GTNKRAP | 215 | ALWYSNLWV | 239 |
| | | TSNYAN | | | | | |
| CD3-52 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-53 | VH | TYAMN | 194 | RIRSKYNNYATYYA | 223 | HGNFGNSYVS | 258 |
| | | | | DSVKG | | WFDY | |
| | VL | GSSTGAVT | 204 | GTNKRAP | 215 | ALWYSNLWV | 239 |
| | | TSNYAN | | | | | |
| CD3-54 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-55 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-56 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-57 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-58 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-59 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-60 | VH | TYAMN | 194 | RIRSKYNNYATYYA | 223 | HGNFGNSYVS | 238 |
| | | | | DSVKG | | WFAY | |
| | VL | GSSTGAVT | 209 | GTNKRAP | 215 | ALWYSNLWV | 239 |
| | | SSNYAN | | | | | |
| CD3-61 | VH | TYAMN | 194 | RIRSKYNNYATYYA | 223 | HGNFGNSYVS | 238 |
| | | | | DSVKG | | WFAY | |
| | VL | GSSTGAVT | 210 | GTNKRAP | 215 | ALWYSNLWV | 239 |
| | | SGHYAN | | | | | |
| CD3-62 | VH | TYAMN | 194 | RIRSKYNNYATYYA | 223 | HGNFGNSYVS | 238 |
| | | | | DSVKG | | WFAY | |
| | VL | GSSTGAVT | 204 | DTNKRAP | 231 | ALWYSNLWV | 239 |
| | | TSNYAN | | | | | |
| CD3-63 | VH | TYAMN | 194 | RIRSKYNNYATYYA | 223 | HGNFGNSYVS | 238 |
| | | | | DSVKG | | WFAY | |
| | VL | GSSTGAVT | 204 | GTNNRAP | 232 | ALWYSNLWV | 239 |
| | | TSNYAN | | | | | |
| CD3-64 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAS | 233 | ALWYSNLWV | 239 |
| | | TSNYAN | | | | | |
| CD3-65 | VH | TYAMN | 194 | RIRSKYNNYATYYA | 223 | HGNFGNSYVS | 238 |
| | | | | DSVKG | | WFAY | |
| | VL | GSSTGAVT | 204 | GTSNKHS | 234 | ALWYSNLWV | 239 |
| | | TSNYAN | | | | | |
| CD3-66 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-67 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-68 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-69 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-70 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-71 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-72 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-73 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | LLWYSNLWV | 259 |
| | | TSNYAN | | | | | |
| CD3-74 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNHWV | 250 |
| | | TSNYAN | | | | | |
| CD3-75 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-76 | VH | TYAMN | 194 | RIRSKYNNYATYYA | 223 | HGNFGNSYVS | 238 |
| | | | | DSVKG | | WFAY | |
| | VL | RSSTGAVT | 195 | GTNKRAP | 215 | ALWYSNLWV | 239 |
| | | TSNYAN | | | | | |
| CD3-77 | VH | TYAMN | 194 | RIRSKYNNYATYYA | 223 | HGNFGNSYVS | 238 |
| | | | | DSVKG | | WFAY | |
| | VL | KSSTGAVT | 211 | GTNKRAP | 215 | ALWYSNLWV | 239 |
| | | TSNYAN | | | | | |
| CD3-78 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNHWV | 250 |
| | | TSNYAN | | | | | |
| CD3-79 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-80 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-81 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-82 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-83 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-84 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-85 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-86 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-87 | VH | TYAMN | 194 | RIRSKYNNYATYYA | 223 | HGNFGDSYVS | 249 |
| | | | | DSVKG | | WFAY | |
| | VL | GSSTGAVT | 204 | GTNKRAP | 215 | ALWYSNLWV | 239 |
| | | TSNYAN | | | | | |
| CD3-88 | VH | TYAMN | 194 | RIRSKYNNYATYYA | 223 | HGNFGDSYVS | 249 |
| | | | | DSVKG | | WFAY | |
| | VL | GSSTGAVT | 204 | GTNKRAP | 215 | ALWYSNLWV | 239 |
| | | TSNYAN | | | | | |
| CD3-89 | VH | TYAMN | 194 | RIRSKANNYATYYA | 227 | HGNFGDSYVS | 249 |
| | | | | DSVKG | | WFAY | |
| | VL | GSSTGAVT | 204 | GTNKRAP | 215 | ALWYSNLWV | 239 |
| | | TSNYAN | | | | | |
| CD3-90 | VH | TYAMN | 194 | RIRSKYNNYATYYA | 223 | HGNFGDSYVS | 256 |
| | | | | DSVKG | | WFDY | |
| | VL | GSSTGAVT | 204 | GTNKRAP | 215 | ALWYSNLWV | 239 |
| | | TSNYAN | | | | | |
| CD3-91 | VH | TYAMS | 207 | RIRSKANNYATYYA | 227 | HGNFGDSYVS | 256 |
| | | | | DSVKG | | WFDY | |
| | VL | GSSTGAVT | 204 | GTNKRAP | 215 | ALWYSNLWV | 239 |
| | | TSNYAN | | | | | |
| CD3-92 | VH | TYAMN | 194 | RIRSNGGYSTYYA | 235 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-93 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-94 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-95 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNLWV | 239 |
| | | TSNYAN | | | | | |
| CD3-96 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNHWV | 250 |
| | | TSNYAN | | | | | |
| CD3-97 | VH | TYAMN | 194 | RIRSKYNNYATYYA | 223 | HGNFGDSYVS | 249 |
| | | | | DSVKG | | WFAY | |
| | VL | GSSTGAVT | 204 | GTNKRAP | 215 | ALWYSNHWV | 250 |
| | | TSNYAN | | | | | |
| CD3-98 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNHWV | 250 |
| | | TSNYAN | | | | | |
| CD3-99 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNHWV | 250 |
| | | TSNYAN | | | | | |
| CD3-100 | VH | TYAMN | 194 | RIRSKYNNYATYYA | 223 | HGNFGDSYVS | 249 |
| | | | | DSVKG | | WFAY | |
| | VL | GSSTGAVT | 204 | GTNKRAP | 215 | ALWYSNHWV | 250 |
| | | TSNYAN | | | | | |
| CD3-101 | VH | TYAMN | 194 | RIRSKYNNYATYYA | 223 | HGNFGDSYVS | 249 |
| | | | | DSVKG | | WFAY | |
| | VL | GSSTGAVT | 204 | GTNKRAP | 215 | ALWYSNHWV | 250 |
| | | TSNYAN | | | | | |
| CD3-102 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNHWV | 250 |
| | | TSNYAN | | | | | |
| CD3-103 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNHWV | 250 |
| | | TSNYAN | | | | | |
| CD3-104 | VH | TYAMN | 194 | RIRSKYNNYATYYA | 223 | HGNFGDSYVS | 249 |
| | | | | DSVKG | | WFAY | |
| | VL | GSSTGAVT | 204 | GTNKRAP | 215 | ALWYSNHWV | 250 |
| | | TSNYAN | | | | | |
| CD3-105 | VH | TYAMN | 194 | RIRSKYNNYATYYA | 223 | HGNFGDSYVS | 249 |
| | | | | DSVKG | | WFAY | |
| | VL | GSSTGAVT | 204 | GTNKRAP | 215 | ALWYSNHWV | 250 |
| | | TSNYAN | | | | | |
| CD3-106 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNHWV | 250 |
| | | TSNYAN | | | | | |
| CD3-107 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNHWV | 250 |
| | | TSNYAN | | | | | |
| CD3-108 | VH | TYAMN | 194 | RIRSKYNNYATYYA | 223 | HGNFGDSYVS | 249 |
| | | | | DSVKG | | WFAY | |
| | VL | GSSTGAVT | 204 | GTNKRAP | 215 | ALWYSNHWV | 250 |
| | | TSNYAN | | | | | |
| CD3-109 | VH | TYAMN | 194 | RIRSKYNNYATYYA | 223 | HGNFGDSYVS | 249 |
| | | | | DSVKG | | WFAY | |
| | VL | GSSTGAVT | 204 | GTNKRAP | 215 | ALWYSNHWV | 250 |
| | | TSNYAN | | | | | |
| CD3-110 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNHWV | 250 |
| | | TSNYAN | | | | | |
| CD3-111 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNHWV | 250 |
| | | TSNYAN | | | | | |
| CD3-112 | VH | TYAMN | 194 | RIRSKYNNYATYYA | 223 | HGNFGDSYVS | 249 |
| | | | | DSVKG | | WFAY | |
| | VL | GSSTGAVT | 204 | GTNKRAP | 215 | ALWYSNHWV | 250 |
| | | TSNYAN | | | | | |
| CD3-113 | VH | TYAMN | 194 | RIRSKYNNYATYYA | 223 | HGNFGDSYVS | 249 |
| | | | | DSVKG | | WFAY | |
| | VL | GSSTGAVT | 204 | GTNKRAP | 215 | ALWYSNHWV | 250 |
| | | TSNYAN | | | | | |
| CD3-114 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNHWV | 250 |
| | | TSNYAN | | | | | |
| CD3-115 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNHWV | 250 |
| | | TSNYAN | | | | | |
| CD3-116 | VH | TYAMN | 194 | RIRSKYNNYATYYA | 223 | HGNFGDSYVS | 249 |
| | | | | DSVKG | | WFAY | |
| | VL | GSSTGAVT | 204 | GTNKRAP | 215 | ALWYSNHWV | 250 |
| | | TSNYAN | | | | | |
| CD3-117 | VH | TYAMN | 194 | RIRSKYNNYATYYA | 223 | HGNFGDSYVS | 249 |
| | | | | DSVKG | | WFAY | |
| | VL | GSSTGAVT | 204 | GTNKRAP | 215 | ALWYSNHWV | 250 |
| | | TSNYAN | | | | | |
| CD3-118 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNHWV | 250 |
| | | TSNYAN | | | | | |
| CD3-119 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNHWV | 250 |
| | | TSNYAN | | | | | |
| CD3-120 | VH | TYAMN | 194 | RIRSKYNNYATYYA | 223 | HGNFGDSYVS | 249 |
| | | | | DSVKG | | WFAY | |
| | VL | GSSTGAVT | 204 | GTNKRAP | 215 | ALWYSNHWV | 250 |
| | | TSNYAN | | | | | |
| CD3-121 | VH | TYAMN | 194 | RIRSKYNNYATYYA | 223 | HGNFGDSYVS | 249 |
| | | | | DSVKG | | WFAY | |
| | VL | GSSTGAVT | 204 | GTNKRAP | 215 | ALWYSNHWV | 250 |
| | | TSNYAN | | | | | |
| CD3-122 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNHWV | 250 |
| | | TSNYAN | | | | | |
| CD3-123 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNHWV | 250 |
| | | TSNYAN | | | | | |
| CD3-124 | VH | TYAMN | 194 | RIRSKYNNYATYYA | 223 | HGNFGDSYVS | 249 |
| | | | | DSVKG | | WFAY | |
| | VL | GSSTGAVT | 204 | GTNKRAP | 215 | ALWYSNHWV | 250 |
| | | TSNYAN | | | | | |
| CD3-125 | VH | TYAMN | 194 | RIRSKYNNYATYYA | 223 | HGNFGDSYVS | 249 |
| | | | | DSVKG | | WFAY | |
| | VL | GSSTGAVT | 204 | GTNKRAP | 215 | ALWYSNHWV | 250 |
| | | TSNYAN | | | | | |
| CD3-126 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNHWV | 250 |
| | | TSNYAN | | | | | |
| CD3-127 | VH | TYAMN | 194 | RIRSKYNNYATYYA | 223 | HGNFGNSYVS | 238 |
| | | | | DSVKG | | WFAY | |
| | VL | GSSTGAVT | 204 | GTNKRAP | 215 | ALWYSNHWV | 250 |
| | | TSNYAN | | | | | |
| CD3-128 | VH | TYAMN | 194 | RIRSKYNNYATYYA | 223 | HGNFGDSYVS | 249 |
| | | | | DSVKG | | WFAY | |
| | VL | GSSTGAVT | 204 | GTNKRAP | 215 | ALWYSNHWV | 250 |
| | | TSNYAN | | | | | |
| CD3-129 | VH | TYAMN | 194 | RIRSKYNNYATYYAD | 214 | HGNFGNSYVS | 260 |
| | | | | SVKD | | WFAH | |
| | VL | GSSTGAVTS | 209 | GTNKRAP | 215 | ALWYSNLWV | 239 |
| | | SNYAN | | | | | |
| CD3-130 | VH | TYAMN | 194 | RIRSKYNNYATYYAD | 214 | HGNFGNSYVS | 238 |
| | | | | SVKD | | WFAY | |
| | VL | RSSTGAVTT | 195 | GTNKRAP | 215 | ALWYSNLWV | 239 |
| | | SNYAN | | | | | |

TABLE 22C
CD3 Binders-CDR sequences according to Chothia numbering scheme

| Binding | | | SEQ ID | | SEQ ID | | SEQ ID |
|---|---|---|---|---|---|---|---|
| Domain | Chain | CDR1 | NO: | CDR2 | NO: | CDR3 | NO: |
| CD3-1 | VH | GYTFTRY | 261 | NPSRGY | 274 | YYDDHYCLDY | 236 |
| | VL | SSSVSY | 262 | DTS | 275 | WSSNPF | 286 |
| CD3-2 | VH | GFTFNTY | 263 | RSKYNN | 276 | HGNFGNSYVS | 238 |
| | | | | YA | | WFAY | |
| | VL | STGAVTTSN | 264 | GTN | 277 | WYSNLW | 287 |
| | | Y | | | | | |
| CD3-3 | VH | GYTFTSY | 265 | NPSSGY | 278 | WQDYDVYFDY | 240 |
| | VL | SSSVSY | 262 | ATS | 279 | WSSNPP | 288 |
| CD3-4 | VH | GYTFTRY | 261 | NPSRGY | 274 | YYDDHYCLDY | 236 |
| | VL | SSSVSY | 262 | DTS | 275 | WSSNPL | 289 |
| CD3-5 | VH | GYTFTRY | 261 | NPSRGY | 274 | YYDDHYCLDY | 236 |
| | VL | SSSVSY | 262 | DTS | 275 | WSSNPF | 286 |
| CD3-6 | VH | GFKFSGY | 266 | WYDGSK | 280 | QMGYWHFDL | 243 |
| | VL | SQSVSSY | 267 | DAS | 281 | RSNWPPL | 290 |
| CD3-7 | VH | GFTFSTY | 268 | RSKYNN | 282 | HGNFGNSYVS | 291 |
| | | | | YAT | | WFA | |
| | VL | STGAVTTSN | 264 | GTN | 277 | WYSNLW | 287 |
| | | Y | | | | | |
| CD3-8 | VH | GYTFTRY | 261 | NPSRGY | 274 | YYDDHYCLDY | 236 |
| | VL | SSSVSY | 262 | DTS | 275 | WSSNPL | 289 |
| CD3-9 | VH | GFTFNTY | 263 | RSKYNN | 282 | HGNFGNSYVS | 291 |
| | | | | YAT | | WFA | |
| | VL | STGAVTTSN | 264 | GTN | 277 | WYSNLW | 287 |
| | | Y | | | | | |
| CD3-10 | VH | GFTFNTY | 263 | RSKYNN | 276 | HGNFGNSYVS | 238 |
| | | | | YA | | WFAY | |
| | VL | STGAVTTSN | 264 | GTN | 277 | WYSNLW | 287 |
| | | Y | | | | | |
| CD3-11 | VH | GFTFNSY | 269 | RSKYNN | 276 | HGNFGNSYVS | 246 |
| | | | | YA | | WWAY | |
| | VL | STGAVTSGN | 270 | GTK | 283 | WYSNRW | 292 |
| | | Y | | | | | |
| CD3-12 | VH | GFTFNKY | 271 | RSKYNN | 276 | HGNFGNSYISY | 248 |
| | | | | YA | | WAY | |
| | VL | STGAVTSGN | 270 | GTK | 283 | WYSNRW | 292 |
| | | Y | | | | | |
| CD3-13 | VH | GYTFTRY | 261 | NPSRGY | 274 | YYDDHYCLDY | 236 |
| | VL | SSSVSY | 262 | DTS | 275 | WSSNPF | 286 |
| CD3-14 | VH | GFTFSTY | 268 | RSKYNN | 276 | HGNFGNSYVS | 238 |
| | | | | YA | | WFAY | |
| | VL | STGAVTTSN | 264 | GTN | 277 | WYSNLW | 287 |
| | | Y | | | | | |
| CD3-15 | VH | GFTFNTY | 263 | RSKYNN | 276 | HGNFGNSYVS | 238 |
| | | | | YA | | WFAY | |
| | VL | STGAVTTSN | 264 | GTN | 277 | WYSNLW | 287 |
| | | Y | | | | | |
| CD3-16 | VH | GFTFNTY | 263 | RSKYNN | 276 | HGNFGNSYVS | 238 |
| | | | | YA | | WFAY | |
| | VL | STGAVTTSN | 264 | GTN | 277 | WYSNLW | 287 |
| | | Y | | | | | |
| CD3-17 | VH | GFTFSTY | 268 | RSKYNN | 276 | HGNFGDSYVS | 249 |
| | | | | YA | | WFAY | |
| | VL | STGAVTTSN | 264 | GTN | 277 | WYSNHW | 293 |
| | | Y | | | | | |
| CD3-18 | VH | GYTFTRY | 261 | NPSRGY | 274 | YYDDHYCLDY | 236 |
| | VL | SSSVSY | 262 | DTS | 275 | WSSNPF | 286 |
| CD3-19 | VH | GYTFTRY | 261 | NPSRGY | 274 | YYDDHYSLDY | 251 |
| | VL | SSSVSY | 262 | DTS | 275 | WSSNPF | 286 |
| CD3-20 | VH | GYSFTGY | 272 | NPYKGV | 284 | SGYYGDSDWY | 252 |
| | | | | | | FDV | |
| | VL | SQDIRNY | 273 | YTS | 285 | GNTLPW | 294 |
| CD3-21 | VH | GFTFNTY | 263 | RSKYNN | 276 | HGNFGNSYVS | 238 |
| | | | | YA | | WFAY | |
| | VL | RSSTGAVTT | 195 | GTNKRA | 215 | ALWYSNLWV | 239 |
| | | SNYAN | | P | | | |

TABLE 22D
CD3 Binders - CDR sequences according to combination of Kabat and Chothia
numbering schemes

| Binding | | | SEQ ID | | SEQ ID | | SEQ ID |
|---|---|---|---|---|---|---|---|
| Domain | Chain | CDR1 | NO: | CDR2 | NO: | CDR3 | NO: |
| CD3-1 | VH | GYTFTRY | 295 | YINPSRGYTNYN | 212 | YYDDHYCLDY | 236 |
| | | TMH | | QKFKD | | | |
| | VL | SASSSVS | 193 | DTSKLAS | 213 | QQWSSNPFT | 237 |
| | | YMN | | | | | |
| CD3-2 | VH | GFTFNTY | 296 | RIRSKYNNYATYY | 214 | HGNFGNSYV | 238 |
| | | AMN | | ADSVKD | | SWFAY | |
| | VL | RSSTGAV | 195 | GTNKRAP | 215 | ALWYSNLWV | 239 |
| | | TTSNYAN | | | | | |
| CD3-3 | VH | GYTFTSY | 297 | YINPSSGYTKYN | 216 | WQDYDVYFD | 240 |
| | | TMH | | QKFKD | | Y | |
| | VL | RASSSVS | 197 | ATSNLAS | 217 | QQWSSNPPT | 241 |
| | | YMH | | | | | |
| CD3-4 | VH | GYTFTRY | 295 | YINPSRGYTNYN | 212 | YYDDHYCLDY | 236 |
| | | TMH | | QKFKD | | | |
| | VL | RASSSVS | 198 | DTSKVAS | 218 | QQWSSNPLT | 242 |
| | | YMN | | | | | |
| CD3-5 | VH | GYTFTRY | 295 | YINPSRGYTNYN | 219 | YYDDHYCLDY | 236 |
| | | TMH | | QKVKD | | | |
| | VL | SASSSVS | 193 | DTSKLAS | 213 | QQWSSNPFT | 237 |
| | | YMN | | | | | |
| CD3-6 | VH | GFKFSGY | 298 | VIWYDGSKKYYV | 220 | QMGYWHFDL | 243 |
| | | GMH | | DSVKG | | | |
| | VL | RASQSVS | 200 | DASNRAT | 221 | QQRSNWPPL | 244 |
| | | SYLA | | | | T | |
| CD3-7 | VH | GFTFSTY | 299 | RIRSKYNNYATYY | 303 | HGNFGNSYV | 238 |
| | | AMN | | ADSVK | | SWFAY | |
| | VL | RSSTGAV | 195 | GTNKRAP | 215 | ALWYSNLWV | 239 |
| | | TTSNYAN | | | | | |
| CD3-8 | VH | GYTFTRY | 295 | YINPSRGYTNYN | 212 | YYDDHYCLDY | 236 |
| | | TMH | | QKFKD | | | |
| | VL | RASSSVS | 198 | DTSKVAS | 218 | QQWSSNPLT | 242 |
| | | YMN | | | | | |
| CD3-9 | VH | GFTFNTY | 296 | RIRSKYNNYATYY | 303 | HGNFGNSYV | 238 |
| | | AMN | | ADSVK | | SWFAY | |
| | VL | RSSTGAV | 195 | GTNKRAP | 215 | ALWYSNLWV | 239 |
| | | TTSNYAN | | | | | |
| CD3-10 | VH | GFTFNTY | 296 | RIRSKYNNYATYY | 214 | HGNFGNSYV | 238 |
| | | AMN | | ADSVKD | | SWFAY | |
| | VL | RSSTGAV | 195 | GTNKRAP | 215 | ALWYSNLWV | 239 |
| | | TTSNYAN | | | | | |
| CD3-11 | VH | GFTFNSY | 300 | RIRSKYNNYATYY | 223 | HGNFGNSYV | 246 |
| | | AMN | | ADSVKG | | SWWAY | |
| | VL | GSSTGAV | 202 | GTKFLAP | 224 | VLWYSNRWV | 247 |
| | | TSGNYPN | | | | | |
| CD3-12 | VH | GFTFNKY | 301 | RIRSKYNNYATYY | 214 | HGNFGNSYIS | 248 |
| | | AMN | | ADSVKD | | YWAY | |
| | VL | GSSTGAV | 202 | GTKFLAP | 224 | VLWYSNRWV | 247 |
| | | TSGNYPN | | | | | |
| CD3-13 | VH | GYTFTRY | 295 | YINPSRGYTNYN | 212 | YYDDHYCLDY | 236 |
| | | TMH | | QKFKD | | | |
| | VL | SASSSVS | 193 | DTSKLAS | 213 | QQWSSNPFT | 237 |
| | | YMN | | | | | |
| CD3-14 | VH | GFTFSTY | 299 | RIRSKYNNYATYY | 214 | HGNFGNSYV | 238 |
| | | AMN | | ADSVKD | | SWFAY | |
| | VL | RSSTGAV | 195 | GTNKRAP | 215 | ALWYSNLWV | 239 |
| | | TTSNYAN | | | | | |
| CD3-15 | VH | GFTFNTY | 296 | RIRSKYNNYATYY | 214 | HGNFGNSYV | 238 |
| | | AMN | | ADSVKD | | SWFAY | |
| | VL | RSSTGAV | 195 | GTNKRAP | 215 | ALWYSNLWV | 239 |
| | | TTSNYAN | | | | | |
| CD3-16 | VH | GFTFNTY | 296 | RIRSKYNNYATYY | 223 | HGNFGNSYV | 238 |
| | | AMN | | ADSVKG | | SWFAY | |
| | VL | GSSTGAV | 204 | GTNKRAP | 215 | ALWYSNLWV | 239 |
| | | TTSNYAN | | | | | |
| CD3-17 | VH | GFTFSTY | 299 | RIRSKYNNYATYY | 223 | HGNFGDSYV | 249 |
| | | AMN | | ADSVKG | | SWFAY | |
| | VL | GSSTGAV | 204 | GTNKRAP | 215 | ALWYSNHWV | 250 |
| | | TTSNYAN | | | | | |
| CD3-18 | VH | GYTFTRY | 295 | YINPSRGYTNYN | 219 | YYDDHYCLDY | 236 |
| | | TMH | | QKVKD | | | |
| | VL | SASSSVS | 193 | DTSKLAS | 213 | QQWSSNPFT | 237 |
| | | YMN | | | | | |
| CD3-19 | VH | GYTFTRY | 295 | YINPSRGYTNYN | 219 | YYDDHYSLDY | 251 |
| | | TMH | | QKVKD | | | |
| | VL | SASSSVS | 193 | DTSKLAS | 213 | QQWSSNPFT | 237 |
| | | YMN | | | | | |
| CD3-20 | VH | GYSFTGY | 302 | LINPYKGVSTYNQ | 225 | SGYYGDSDW | 252 |
| | | TMN | | KFKD | | YFDV | |
| | VL | RASQDIR | 206 | YTSRLHS | 304 | QQGNTLPWT | 253 |
| | | NYLN | | | | | |

In some embodiments, a MBM (e.g., a BBM) can comprise a CD3 ABM which comprises the CDRs of any of CD3-1 to CD3-130 as defined by Kabat numbering (e.g., as set forth in Table 22B). In other embodiments, a MBM (e.g., a BBM) can comprise a CD3 ABM which comprises the CDRs of any of CD3-1 to CD3-130 as defined by Chothia numbering (e.g., as set forth in Table 22C). In yet other embodiments, a MBM (e.g., a BBM) can comprise a CD3 ABM which comprises the CDRs of any of CD3-1 to CD3-130 as defined by a combination of Kabat and Chothia numbering (e.g., as set forth in Table 22D).

In some embodiments, a CD3 ABM comprises the CDR sequences of CD3-1. In some embodiments, a CD3 ABM comprises the CDR sequences of CD3-2. In some embodiments, a CD3 ABM comprises the CDR sequences of CD3-3. In some embodiments, a CD3 ABM comprises the CDR sequences of CD3-4. In some embodiments, a CD3 ABM comprises the CDR sequences of CD3-5. In some embodiments, a CD3 ABM comprises the CDR sequences of CD3-6. In some embodiments, a CD3 ABM comprises the CDR sequences of CD3-7. In some embodiments, a CD3 ABM comprises the CDR sequences of CD3-8. In some embodiments, a CD3 ABM comprises the CDR sequences of CD3-9. In some embodiments, a CD3 ABM comprises the CDR sequences of CD3-10. In some embodiments, a CD3 ABM comprises the CDR sequences of CD3-11. In some embodiments, a CD3 ABM comprises the CDR sequences of CD3-12. In some embodiments, a CD3 ABM comprises the CDR sequences of CD3-13. In some embodiments, a CD3 ABM comprises the CDR sequences of CD3-14. In some embodiments, a CD3 ABM comprises the CDR sequences of CD3-15. In some embodiments, a CD3 ABM comprises the CDR sequences of CD3-16. In some embodiments, a CD3 ABM comprises the CDR sequences of CD3-17. In some embodiments, a CD3 ABM comprises the CDR sequences of CD3-18. In some embodiments, a CD3 ABM comprises the CDR sequences of CD3-19. In some embodiments, a CD3 ABM comprises the CDR sequences of CD3-20. In some embodiments, a CD3 ABM comprises the CDR sequences of CD3-21. In some embodiments, a CD3 ABM comprises the CDR sequences of CD3-22. In some embodiments, a CD3 ABM comprises the CDR sequences of CD3-23. In some embodiments, a CD3 ABM comprises the CDR sequences of CD3-24. In some embodiments, a CD3 ABM comprises the CDR sequences of CD3-25. In some embodiments, a CD3 ABM comprises the CDR sequences of CD3-26. In some embodiments, a CD3 ABM comprises the CDR sequences of CD3-27. In some embodiments, a CD3 ABM comprises the CDR sequences of CD3-28. In some embodiments, a CD3 ABM comprises the CDR sequences of CD3-29. In some embodiments, a CD3 ABM comprises the CDR sequences of CD3-30. In some embodiments, a CD3 ABM comprises the CDR sequences of CD3-31. In some embodiments, a CD3 ABM comprises the CDR sequences of CD3-32. In some embodiments, a CD3 ABM comprises the CDR sequences of CD3-33. In some embodiments, a CD3 ABM comprises the CDR sequences of CD3-34. In some embodiments, a CD3 ABM comprises the CDR sequences of CD3-35. In some embodiments, a CD3 ABM comprises the CDR sequences of CD3-36. In some embodiments, a CD3 ABM comprises the CDR sequences of CD3-37. In some embodiments, a CD3 ABM comprises the CDR sequences of CD3-38. In some embodiments, a CD3 ABM comprises the CDR sequences of CD3-39. In some embodiments, a CD3 ABM comprises the CDR sequences of CD3-40. In some embodiments, a CD3 ABM comprises the CDR sequences of CD3-41. In some embodiments, a CD3 ABM comprises the CDR sequences of CD3-42. In some embodiments, a CD3 ABM comprises the CDR sequences of CD3-43. In some embodiments, a CD3 ABM comprises the CDR sequences of CD3-44. In some embodiments, a CD3 ABM comprises the CDR sequences of CD3-45. In some embodiments, a CD3 ABM comprises the CDR sequences of CD3-46. In some embodiments, a CD3 ABM comprises the CDR sequences of CD3-47. In some embodiments, a CD3 ABM comprises the CDR sequences of CD3-48. In some embodiments, a CD3 ABM comprises the CDR sequences of CD3-49. In some embodiments, a CD3 ABM comprises the CDR sequences of CD3-50. In some embodiments, a CD3 ABM comprises the CDR sequences of CD3-51. In some embodiments, a CD3 ABM comprises the CDR sequences of CD3-52. In some embodiments, a CD3 ABM comprises the CDR sequences of CD3-53. In some embodiments, a CD3 ABM comprises the CDR sequences of CD3-54. In some embodiments, a CD3 ABM comprises the CDR sequences of CD3-55. In some embodiments, a CD3 ABM comprises the CDR sequences of CD3-56. In some embodiments, a CD3 ABM comprises the CDR sequences of CD3-57. In some embodiments, a CD3 ABM comprises the CDR sequences of CD3-58. In some embodiments, a CD3 ABM comprises the CDR sequences of CD3-59. In some embodiments, a CD3 ABM comprises the CDR sequences of CD3-60. In some embodiments, a CD3 ABM comprises the CDR sequences of CD3-61. In some embodiments, a CD3 ABM comprises the CDR sequences of CD3-62. In some embodiments, a CD3 ABM comprises the CDR sequences of CD3-63. In some embodiments, a CD3 ABM comprises the CDR sequences of CD3-64. In some embodiments, a CD3 ABM comprises the CDR sequences of CD3-65. In some embodiments, a CD3 ABM comprises the CDR sequences of CD3-66. In some embodiments, a CD3 ABM comprises the CDR sequences of CD3-67. In some embodiments, a CD3 ABM comprises the CDR sequences of CD3-68. In some embodiments, a CD3 ABM comprises the CDR sequences of CD3-69. In some embodiments, a CD3 ABM comprises the CDR sequences of CD3-70. In some embodiments, a CD3 ABM comprises the CDR sequences of CD3-71. In some embodiments, a CD3 ABM comprises the CDR sequences of CD3-72. In some embodiments, a CD3 ABM comprises the CDR sequences of CD3-73. In some embodiments, a CD3 ABM comprises the CDR sequences of CD3-74. In some embodiments, a CD3 ABM comprises the CDR sequences of CD3-75. In some embodiments, a CD3 ABM comprises the CDR sequences of CD3-76. In some embodiments, a CD3 ABM comprises the CDR sequences of CD3-77. In some embodiments, a CD3 ABM comprises the CDR sequences of CD3-78. In some embodiments, a CD3 ABM comprises the CDR sequences of CD3-79. In some embodiments, a CD3 ABM comprises the CDR sequences of CD3-80. In some embodiments, a CD3 ABM comprises the CDR sequences of CD3-81. In some embodiments, a CD3 ABM comprises the CDR sequences of CD3-82. In some embodiments, a CD3 ABM comprises the CDR sequences of CD3-83. In some embodiments, a CD3 ABM comprises the CDR sequences of CD3-84. In some embodiments, a CD3 ABM comprises the CDR sequences of CD3-85. In some embodiments, a CD3 ABM comprises the CDR sequences of CD3-86. In some embodiments, a CD3 ABM comprises the CDR sequences of CD3-87. In some embodiments, a CD3 ABM comprises the CDR sequences of CD3-88. In some embodiments, a CD3 ABM comprises the CDR sequences of CD3-89. In some embodiments, a CD3 ABM comprises the CDR sequences of CD3-90. In some embodiments, a CD3 ABM comprises the CDR sequences of CD3-91. In some embodiments, a CD3 ABM comprises the CDR sequences of CD3-92. In some embodiments, a CD3 ABM comprises the CDR sequences of CD3-93. In some embodiments, a CD3 ABM comprises the CDR sequences of CD3-94. In some embodiments, a CD3 ABM comprises the CDR sequences of CD3-95. In some embodiments, a CD3 ABM comprises the CDR sequences of CD3-96. In some embodiments, a CD3 ABM comprises the CDR sequences of CD3-97. In some embodiments, a CD3 ABM comprises the CDR sequences of CD3-98. In some embodiments, a CD3 ABM comprises the CDR sequences of CD3-99. In some embodiments, a CD3 ABM comprises the CDR sequences of CD3-100. In some embodiments, a CD3 ABM comprises the CDR sequences of CD3-101. In some embodiments, a CD3 ABM comprises the CDR sequences of CD3-102. In some embodiments, a CD3 ABM comprises the CDR sequences of CD3-103. In some embodiments, a CD3 ABM comprises the CDR sequences of CD3-104. In some embodiments, a CD3 ABM comprises the CDR sequences of CD3-105. In some embodiments, a CD3 ABM comprises the CDR sequences of CD3-106. In some embodiments, a CD3 ABM comprises the CDR sequences of CD3-107. In some embodiments, a CD3 ABM comprises the CDR sequences of CD3-108. In some embodiments, a CD3 ABM comprises the CDR sequences of CD3-109. In some embodiments, a CD3 ABM comprises the CDR sequences of CD3-110. In some embodiments, a CD3 ABM comprises the CDR sequences of CD3-111. In some embodiments, a CD3 ABM comprises the CDR sequences of CD3-112. In some embodiments, a CD3 ABM comprises the CDR sequences of CD3-113. In some embodiments, a CD3 ABM comprises the CDR sequences of CD3-114. In some embodiments, a CD3 ABM comprises the CDR sequences of CD3-115. In some embodiments, a CD3 ABM comprises the CDR sequences of CD3-116. In some embodiments, a CD3 ABM comprises the CDR sequences of CD3-117. In some embodiments, a CD3 ABM comprises the CDR sequences of CD3-118. In some embodiments, a CD3 ABM comprises the CDR sequences of CD3-119. In some embodiments, a CD3 ABM comprises the CDR sequences of CD3-120. In some embodiments, a CD3 ABM comprises the CDR sequences of CD3-121. In some embodiments, a CD3 ABM comprises the CDR sequences of CD3-122. In some embodiments, a CD3 ABM comprises the CDR sequences of CD3-123. In some embodiments, a CD3 ABM comprises the CDR sequences of CD3-124. In some embodiments, a CD3 ABM comprises the CDR sequences of CD3-125. In some embodiments, a CD3 ABM comprises the CDR sequences of CD3-126. In some embodiments, a CD3 ABM comprises the CDR sequences of CD3-127. In some embodiments, a CD3 ABM comprises the CDR sequences of CD3-128. In some embodiments, a CD3 ABM comprises the CDR sequences of CD3-129. In some embodiments, a CD3 ABM comprises the CDR sequences of CD3-130.

A MBM (e.g., a BBM) can comprise the complete heavy and light variable sequences of any one of CD3-1 to CD3-130. In some embodiments, a MBM comprises a CD3 ABM which comprises the VH and VL sequences of CD3-1. In some embodiments, a MBM comprises a CD3 ABM which comprises the VH and VL sequences of CD3-1. In some embodiments, a MBM comprises a CD3 ABM which comprises the VH and VL sequences of CD3-2. In some embodiments, a MBM comprises a CD3 ABM which comprises the VH and VL sequences of CD3-3. In some embodiments, a MBM comprises a CD3 ABM which comprises the VH and VL sequences of CD3-4. In some embodiments, a MBM comprises a CD3 ABM which comprises the VH and VL sequences of CD3-5. In some embodiments, a MBM comprises a CD3 ABM which comprises the VH and VL sequences of CD3-6. In some embodiments, a MBM comprises a CD3 ABM which comprises the VH and VL sequences of CD3-7. In some embodiments, a MBM comprises a CD3 ABM which comprises the VH and VL sequences of CD3-8. In some embodiments, a MBM comprises a CD3 ABM which comprises the VH and VL sequences of CD3-9. In some embodiments, a MBM comprises a CD3 ABM which comprises the VH and VL sequences of CD3-10. In some embodiments, a MBM comprises a CD3 ABM which comprises the VH and VL sequences of CD3-11. In some embodiments, a MBM comprises a CD3 ABM which comprises the VH and VL sequences of CD3-12. In some embodiments, a MBM comprises a CD3 ABM which comprises the VH and VL sequences of CD3-13. In some embodiments, a MBM comprises a CD3 ABM which comprises the VH and VL sequences of CD3-14. In some embodiments, a MBM comprises a CD3 ABM which comprises the VH and VL sequences of CD3-15. In some embodiments, a MBM comprises a CD3 ABM which comprises the VH and VL sequences of CD3-16. In some embodiments, a MBM comprises a CD3 ABM which comprises the VH and VL sequences of CD3-17. In some embodiments, a MBM comprises a CD3 ABM which comprises the VH and VL sequences of CD3-18. In some embodiments, a MBM comprises a CD3 ABM which comprises the VH and VL sequences of CD3-19. In some embodiments, a MBM comprises a CD3 ABM which comprises the VH and VL sequences of CD3-20. In some embodiments, a MBM comprises a CD3 ABM which comprises the VH and VL sequences of CD3-21. In some embodiments, a MBM comprises a CD3 ABM which comprises the VH and VL sequences of CD3-22. In some embodiments, a MBM comprises a CD3 ABM which comprises the VH and VL sequences of CD3-23. In some embodiments, a MBM comprises a CD3 ABM which comprises the VH and VL sequences of CD3-24. In some embodiments, a MBM comprises a CD3 ABM which comprises the VH and VL sequences of CD3-25. In some embodiments, a MBM comprises a CD3 ABM which comprises the VH and VL sequences of CD3-26. In some embodiments, a MBM comprises a CD3 ABM which comprises the VH and VL sequences of CD3-27. In some embodiments, a MBM comprises a CD3 ABM which comprises the VH and VL sequences of CD3-28. In some embodiments, a MBM comprises a CD3 ABM which comprises the VH and VL sequences of CD3-129. In some embodiments, a MBM comprises a CD3 ABM which comprises the VH and VL sequences of CD3-130.

In addition to the CDR sets described in Tables 22B-22D (i.e., the set of six CDRs for each of CD3-1 to CD3-130), the present disclosure provides variant CDR sets. In one embodiment, a set of 6 CDRs can have 1, 2, 3, 4 or 5 amino acid changes from a CDR set described in Tables 22B-22D, as long as the CD3 ABM is still able to bind to the target antigen, as measured by at least one of a Biacore, surface plasmon resonance (SPR) and/or BLI (biolayer interferometry, e.g., Octet assay) assay.

In addition to the variable heavy and variable light domains disclosed in Table 22A that form an ABM to CD3, the present disclosure provides variant VH and VL domains. In one embodiment, the variant VH and VL domains each can have from 1, 2, 3, 4, 5, 6, 7, 8, 9 or 10 amino acid changes from the VH and VL domain set forth in Table 22A, as long as the ABM is still able to bind to the target antigen, as measured at least one of a Biacore, surface plasmon resonance (SPR) and/or BLI (biolayer interferometry, e.g., Octet assay) assay. In another embodiment, the variant VH and VL are at least 90, 95, 97, 98 or 99% identical to the respective VH or VL disclosed in Table 22A, as long as the ABM is still able to bind to the target antigen, as measured by at least one of a Biacore, surface plasmon resonance (SPR) and/or BLI (biolayer interferometry, e.g., Octet assay) assay.

In some embodiments, a second MBM (e.g., BBM) can comprise an ABM4 which is a CD3 binding molecule or an antigen-binding domain thereof as described in WO 2020/052692, the contents of which are incorporated herein by reference in their entireties. Tables 1A-1J-2 of WO 2020/052692, incorporated herein by reference in their entireties, list exemplary sequences of CD3 binding molecules that can be used in MBMs of the disclosure.

In some embodiments, a MBM comprises a CD3 ABM which comprises CDR-H1, CDR-H2, CDR-H3, CDR-L1, CDR-L2, and CDR-L3 sequences selected from those set forth in Table 1A of WO 2020/052692. In some embodiments, a MBM comprises a CD3 ABM which comprises CDR-H1, CDR-H2, CDR-H3, CDR-L1, CDR-L2, and CDR-L3 sequences selected from those set forth in Table 1B of WO 2020/052692. In some embodiments, a MBM comprises a CD3 ABM which comprises CDR-H1, CDR-H2, CDR-H3, CDR-L1, CDR-L2, and CDR-L3 sequences selected from those set forth in Table 10 of WO 2020/052692. In some embodiments, a MBM comprises a CD3 ABM which comprises CDR-H1, CDR-H2, and CDR-H3 sequences of any one of the binders set forth in Table 1D-1 of WO 2020/052692 and the corresponding CDR-L1, CDR-L2, and CDR-L3 sequences set forth in Table 1D-2 of WO 2020/052692. In some embodiments, a MBM comprises a CD3 ABM which comprises CDR-H1, CDR-H2 and CDR-H3 sequences of any one of the binders set forth in Table 1E-1 of WO 2020/052692 and the corresponding CDR-L1, CDR-L2, and CDR-L3 sequences set forth in Table 1E-2 of WO 2020/052692. In some embodiments, a MBM comprises a CD3 ABM which comprises CDR-H1, CDR-H2, and CDR-H3 sequences of any one of the binders set forth in Table 1F-1 of WO 2020/052692 and the corresponding CDR-L1, CDR-L2, and CDR-L3 sequences set forth in Table 1F-2 of WO 2020/052692. In some embodiments, a MBM comprises a CD3 ABM which comprises CDR-H1, CDR-H2 and CDR-H3 sequences of any one of the binders set forth in Table 1G-1 of WO 2020/052692 and the corresponding CDR-L1, CDR-L2, and CDR-L3 sequences set forth in Table 1G-2 of WO 2020/052692. In some embodiments, a MBM comprises a CD3 ABM which comprises CDR-H1, CDR-H2, and CDR-H3 sequences of any one of the binders set forth in Table 1H-1 of WO 2020/052692 and the corresponding CDR-L1, CDR-L2, and CDR-L3 sequences set forth in Table 1H-2 of WO 2020/052692. In some embodiments, a MBM comprises a CD3 ABM which comprises CDR-H1, CDR-H2, and CDR-H3 sequences of any one of the binders set forth in Table 11-1 of WO 2020/052692 and the corresponding CDR-L1, CDR-L2, and CDR-L3 sequences set forth in Table 11-2 of WO 2020/052692. In some embodiments, a MBM comprises a CD3 ABM which comprises a VH and/or VL sequence of any one of the binders set forth in Tables 1J-1 and 1J-2 of WO 2020/052692.

In some embodiments, the antigen-binding domain that specifically binds to human CD3 is non-immunoglobulin based and is instead derived from a non-antibody scaffold protein, for example one of the non-antibody scaffold proteins described in Section 7.2.2. In an embodiment, the antigen-binding domain that specifically binds to human CD3 comprises Affilin-144160, which is described in WO 2017/013136. Affilin-144160 has the following amino acid sequence:

(SEQ ID NO: 1319)
MQIFVKTLTGKTITLEVEPSDTIENVKAKIQDKEGIPPDQQWLWFAGKQL
EDGRTLSDYNIQKESTLKLWLVDKAAMQIFVYTRTGKTITLEVEPSDTIE
NVKAKIQDKEGIPPDQQRLIWAGKQLEDGRTLSDYNIALESGLHLVLRLR
AA

### 7.9.2. TCR-α/β ABMs

The MBMs of the disclosure (e.g., BBMs) can contain an ABM4 that specifically binds to the TCR-α chain, the TCR-13 chain, or the TCR-αβ dimer. Exemplary anti-TCR-α/β antibodies are known (see, e.g., US 2012/0034221; Borst et al., 1990, Hum Immunol. 29(3):175-88 (describing antibody BMA031)). The VH, VL, and Kabat CDR sequences of antibody BMA031 are provided in Table 23.

TABLE 23
BMA031 sequences

| | | SEQ |
|---|---|---|
| | | ID |
| Domain | Sequence | NO: |
| BMA031 | KASGYKFTSYVMH | 306 |
| CDR-H1 | | |
| BMA031 | YINPYNDVTKYNEKFK | 307 |
| CDR-H2 | | |
| BMA031 | GSYYDYDGFVY | 308 |
| CDR-H3 | | |
| BMA031 | SATSSVSYMH | 309 |
| CDR-L1 | | |
| BMA031 | DTSKLAS | 213 |
| CDR-L2 | | |
| BMA031 | QQWSSNPLT | 242 |
| CDR-L3 | | |
| BMA031 | EVQLQQSGPELVKPGASVKMSCKASGYKFTSYVMHW | 310 |
| VH | VKQKPGQGLEWIGYINPYNDVTKYNEKFKGKATLTSD | |
| | KSSSTAYMELSSLTSEDSAVHYCARGSYYDYDGFVY | |
| | WGQGTLVTVSA | |
| BMA031 | QIVLTQSPAIMSASPGEKVTMTCSATSSVSYMHWYQQ | 311 |
| VL | KSGTSPKRWIYDTSKLASGVPARFSGSGSGTSYSLTIS | |
| | SMEAEDAATYYCQQWSSNPLTFGAGTKLELK | |

In an embodiment, an ABM4 can comprise the CDR sequences of antibody BMA031. In other embodiments, an ABM4 can comprise the VH and VL sequences of antibody BMA031.

### 7.9.3. TCR-γ/δ ABMs

The MBMs (e.g., BBMs) can contain an ABM4 that specifically binds to the TCR-γ chain, the TCR-δ chain, or the TCR-γδ dimer. Exemplary anti-TCR-γ/δ antibodies are known (see, e.g., U.S. Pat. No. 5,980,892 (describing δTCS1, produced by the hybridoma deposited with the ATCC as accession number HB 9578)).

### 7.10. Secondary T-Cell Signaling Molecule ABMs

The second MBMs (e.g., BBMs) can contain an ABM4 that specifically binds to a secondary T-cell signaling molecule. Exemplary secondary T-cell signaling molecules include CD27, CD28, CD30, CD40L, CD150, CD160, CD226, CD244, BTLA, BTN3A1, B7-1, CTLA4, DR3, GITR, HVEM, ICOS, LAG3, LAIR1, LIGHT, OX40, PD1, PDL1, PDL2, TIGIT, TIM1, TIM2, TIM3, VISTA, CD70, and 4-1BB.

ABM4 can comprise, for example, CDR or VH and/or VL sequences of an antibody identified in Table 24.

TABLE 24
Exemplary secondary T-cell signaling molecule antibodies

| Target | Examples of Antibody Name and/or Reference(s) and/or Source |
|---|---|
| CD27 | Any CD27 antibody described in WO/2012/004367, WO/2014/140374, |
| | WO/2011/130434 |
| CD28 | Any CD28 antibody described in WO/2004/041866, U.S. Pat. No. 6,987,171, |
| | WO/2002/030459, WO/2016/054218 |
| CD30 | Any CD30 antibody described in WO/2003/059282, WO/2006/089232, |
| | WO/2007/040653, WO/2007/084672; iratumumab (BMS); brentuximab (Seattle |
| | Genetics) |
| CD40L | Any CD40L antibody described in U.S. Pat. No. 9,228,018, US 2003/0099642, |
| | WO/2013/056068, US 2003/0099642, WO/2016/040571; 24-31 (Biolegend); |
| | ab52750 (Abcam); ab47204 (Abcam); CDP7657 (UCB Pharma); ruplizumab |
| | (Biogen) |
| CD150 | ab82228 (Abcam); ab130423 (Abcam); MA5-29571 (Invitrogen); A12 (7D4) |
| | (Invitrogen); IPO-3 (Invitrogen) |
| CD160 | Any CD160 antibody described in WO/2018/077926; BY55 (BioLegend); RB01 |
| | (R&D Systems) |
| CD226 | ab33397 (Abcam); DX11 (Bio-Rad) |
| CD244 | 2B4.69 (Invitrogen); AB_1279194 (BioLegend) |
| BTLA | Any BTLA antibody described in WO/2011/014438, WO/2010/106051, |
| | WO/2018/213113, WO/2008/076560, WO/2017/144668 |
| BTN3A1 | MAB7136 (R&D Systems); BT3.1 (Novus Biologicals); 4E1B9 (Abcam) |
| B7-1 | Any B7-1 antibody described in WO/2001/030377, WO/2017/105091 |
| CTLA4 | Any CTLA4 antibody described in WO/2007/113648, WO/2016/130986, |
| | WO/2018/165895, WO/2017/198212, WO/2001/054732, WO/2017/106372, |
| | WO/2018/202649, U.S. Pat. No. 7,034,121; ipilimumab (BMS) |
| DR3 | Any DR3 antibody described in U.S. Pat. No. 7,708,996, WO/2011/106707, |
| | U.S. Pat. No. 8,105,589 |
| GITR | Any GITR antibody described in U.S. Pat. No. 7,812,135, WO/2011/028683, |
| | WO/2017/156058, WO/2017/214548, WO/2007/133822, WO/2018/089628, |
| | WO/2017/096189, WO/2018/158658; MK-1248 (Merck); MK-4166 (Merck); AMG |
| | 228 (Amgen); INCAGN01876 (Incyte); BMS-986156 (BMS); TRX518 (Leap |
| | Therapeutics); MEDI1873 (MedImmune); MEDI6469 (MedImmune); GWN323 |
| | (Novartis) |
| HVEM | MAB3561 (R&D Systems); HMHV-1B18 (Absolute Antibody); |
| ICOS | Any ICOS antibody described in WO/2019/122884, WO/2016/154177, |
| | WO/2018/029474, WO/2016/154177, WO/2019/122882, WO/2012/131004, |
| | WO/2018/187613, WO/2012/131004, WO/2018/025221; JTX-2011 (Jounce |
| | Therapeutics); GSK3359609 (GSK); MEDI-570 (MedImmune) |
| LAG3 | Any LAG3 antibody described in WO/2017/015560, WO/2018/185043, |
| | WO/2018/208868, U.S. Pat. No. 9,902,772; LAG525 (Novartis), Sym022 (Symphogen), |
| | relatlimab (BMS), TSR-033 (Tesaro) |
| LAIR1 | AF2664 (R&D Systems); MAB2664 (R&D Systems) |
| LIGHT | Any LIGHT antibody described in WO/2008/027338, WO/2010/111180, |
| | WO/2015/107331, U.S. Pat. No. 8,058,402, U.S. Pat. No. 10,259,880, |
| | US 2012/0100074, U.S. Pat. No. |
| | 9,771,419; AEVI 002 (Aevi Genomic Medicine) |
| OX40 | Any OX40 antibody described in WO/2010/096418, WO/2013/038191, |
| | WO/2018/089628, WO/2018/202649, WO/2013/068563, WO/2016/196228, |
| | WO/2014/148895, WO/2013/008171, WO/2018/002339: MEDI6469 (MedImmune), |
| | PF-04518600 (Pfizer), BMS 986178 (BMS); MOXR0916 (Genentech); MEDI6383 |
| | (Medlmmune); MEDI0562 (Medlmmune); INCAGN01949 (Incyte); GSK3174998 |
| | (GSK) |
| PD1 | Any PD1 antibody described in WO/2018/025221, WO/2018/185043, |
| | WO/2017/125815, WO/2011/110604. WO/2017/133540. WO/2018/068336. |
| | WO/2017/025016, WO/2017/132827, WO/2017/024465, WO/2017/055404, |
| | WO/2016/014688, WO/2016/106159, WO/2018/020476, WO/2018/028383, |
| | WO/2018/053106, WO/2015/112800, WO/2018/034226, WO/2017/166804, |
| | WO/2017/025051, WO/2016/068801, WO/2017/058115, WO/2018/091661, |
| | WO/2017/055547, WO/2014/179664, WO/2010/029435, WO/2010/029434; |
| | pembrolizumab (Merck), nivolumab (BMS), cemiplimab (Regeneron), dostarlimab |
| | (GSK); TSR-042 (Tesaro); sintilimab (Eli Lilly); MEDI0680 (MedImmune); |
| | spartalizumab (Novartis) |
| PDL1 | Any PDL1 antibody described in WO/2019/122882, WO/2019/114235, |
| | WO/2018/162749, WO/2017/020858, WO/2016/111645, WO/2017/020291, |
| | WO/2017/220990, WO/2015/112805, WO/2016/022630, WO/2018/034225, |
| | WO/2017/196867, WO/2017/161976, WO/2016/197367, WO/2018/181064, |
| | WO/2015/181342, WO/2018/080812, WO/2015/195163, WO/2019/129211; |
| | atezolizumab (Genentech); avelumab (Pfizer); durvalumab (MedImmune); |
| | LY3300054 (Eli Lilly) |
| PDL2 | Any PDL2 antibody described in WO/2010/036959, WO/2014/022758, |
| | WO/2017/053250 |
| TIGIT | Any TIGIT antibody described in WO/2018/160704, WO/2016/028656, |
| | WO/2017/152088, WO/2019/023504, WO/2016/106302, WO/2018/033798, |
| | WO/2018/204363, WO/2019/129221, WO/2019/062832, WO/2019/129261, |
| | WO/2017/037707; etigilimab (OncoMed), tiragolumab (Genentech), AB154 (Arcus |
| | Biosciences) |
| TIM1 | Any TIM1 antibody described in WO/2013/078089, WO/2004/084823, |
| | WO/2007/059082 |
| TIM2 | RMT2-14 (Absolute Antibody); RM0134-5B23 (Novus Biologicals); ab86480 |
| | (Abcam) |
| TIM3 | Any TIM3 antibody described in WO/2017/055404, WO/2016/144803, |
| | WO/2019/009727 WO/2018/039020, WO/2011/155607 WO/2016/068802, |
| | WO/2016/068803, WO/2013/006490, WO/2018/013818, WO/2017/178493, |
| | WO/2018/036561, WO/2018/036561, WO/2015/117002, WO/2017/079115, |
| | WO/2019/023410, WO/2018/126233, WO/2017/205721; TSR-022 (Tesaro); |
| | LY3321367 (Eli Lilly); MBG453 (Novartis) |
| VISTA | Any VISTA antibody described in WO/2017/137830, WO/2016/207717, |
| | WO/2015/097536, WO/2019/078699, WO/2017/181109, WO/2017/181139, |
| | WO/2018/237287, WO/2017/175058; HMBD-002 (Hummingbird Bioscience) |
| CD70 | Any CD70 antibody described in WO/2007/038637, WO/2008/074004, |
| | WO/2012/123586, WO/2014/158821, WO/2017/138471, WO/2018/15218, |
| | WO/2019/152705; ARGX-110 (Argenx); SGN-CD70A (Seattle Genetics); MDX- |
| | 1203 (Medarex) |
| 4-1BB | Any 4-1BB antibody described in WO/2019/072274, WO/2004/010947, |
| | WO/2005/035584, WO/2019/091436, WO/1996/029348, WO/2000/029445, |
| | WO/1996/032495, WO/2017/130076, WO/2015/179236; utomilumab (Pfizer); |
| | urelumab (BMS) |

In some embodiments, ABM4 specifically binds to CD27. In some embodiments, ABM4 specifically binds to CD28. In some embodiments, ABM4 specifically binds to CD30. In some embodiments, ABM4 specifically binds to CD40L. In some embodiments, ABM4 specifically binds to CD150. In some embodiments, ABM4 specifically binds to CD160. In some embodiments, ABM4 specifically binds to CD226. In some embodiments, ABM4 specifically binds to CD244. In some embodiments, ABM4 specifically binds to BTLA. In some embodiments, ABM4 specifically binds to BTN3A1. In some embodiments, ABM4 specifically binds to B7-1. In some embodiments, ABM4 specifically binds to CTLA4. In some embodiments, ABM4 specifically binds to DR3. In some embodiments, ABM4 specifically binds to GITR. In some embodiments, ABM4 specifically binds to HVEM. In some embodiments, ABM4 specifically binds to ICOS. In some embodiments, ABM4 specifically binds to LAG3. In some embodiments, ABM4 specifically binds to LAIR1. In some embodiments, ABM4 specifically binds to LIGHT. In some embodiments, ABM4 specifically binds to OX40. In some embodiments, ABM4 specifically binds to PD1. In some embodiments, ABM4 specifically binds to PDL1. In some embodiments, ABM4 specifically binds to PDL2. In some embodiments, ABM4 specifically binds to TIGIT. In some embodiments, ABM4 specifically binds to TIM1. In some embodiments, ABM4 specifically binds to TIM2. In some embodiments, ABM4 specifically binds to TIM3. In some embodiments, ABM4 specifically binds to VISTA. In some embodiments, ABM4 specifically binds to CD70. In some embodiments, ABM4 specifically binds to 4-1BB.

### 7.11. Nucleic Acids and Host Cells

In another aspect, the disclosure provides nucleic acids (i.e., polynucleotides) encoding the MBMs (e.g., BBMs) of the disclosure. In some embodiments, the MBMs are encoded by a single nucleic acid. In other embodiments, the MBMs are encoded by a plurality (e.g., two, three, four or more) nucleic acids.

A single nucleic acid can encode a MBM that comprises a single polypeptide chain, a MBM that comprises two or more polypeptide chains, or a portion of a MBM that comprises more than two polypeptide chains (for example, a single nucleic acid can encode two polypeptide chains of a BBM comprising three, four or more polypeptide chains, or three polypeptide chains of a BBM comprising four or more polypeptide chains). For separate control of expression, the open reading frames encoding two or more polypeptide chains can be under the control of separate transcriptional regulatory elements (e.g., promoters and/or enhancers). The open reading frames encoding two or more polypeptides can also be controlled by the same transcriptional regulatory elements, and separated by internal ribosome entry site (IRES) sequences allowing for translation into separate polypeptides.

In some embodiments, a MBM comprising two or more polypeptide chains is encoded by two or more nucleic acids. The number of nucleic acids encoding a MBM can be equal to or less than the number of polypeptide chains in the MBM (for example, when more than one polypeptide chains are encoded by a single nucleic acid).

The nucleic acids can be DNA or RNA (e.g., mRNA).

In another aspect, the disclosure provides host cells and vectors containing the nucleic acids of the disclosure. The nucleic acids can be present in a single vector or separate vectors present in the same host cell or separate host cell, as described in more detail herein below.

### 7.11.1. Vectors

The disclosure provides vectors comprising nucleotide sequences encoding a MBM (e.g., a BBM) or a MBM component described herein. In one embodiment, the vectors comprise nucleotides encoding an immunoglobulin-based ABM described herein. In one embodiment, the vectors comprise nucleotides encoding an Fc domain described herein. In one embodiment, the vectors comprise nucleotides encoding a recombinant non-immunoglobulin based ABM described herein. A vector can encode one or more ABMs, one or more Fc domains, one or more non-immunoglobulin based ABM, or any combination thereof (e.g., when multiple components or sub-components are encoded as a single polypeptide chain). In one embodiment, the vectors comprise the nucleotide sequences described herein. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes can be selected by introducing one or more markers which allow for the selection of transfected host cells. The marker can provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by cotransformation. Additional elements may also be needed for optimal synthesis of mRNA. These elements can include splice signals, as well as transcriptional promoters, enhancers, and termination signals.

Once the expression vector or DNA sequence containing the constructs has been prepared for expression, the expression vectors can be transfected or introduced into an appropriate host cell. Various techniques can be employed to achieve this, such as, for example, protoplast fusion, calcium phosphate precipitation, electroporation, retroviral transduction, viral transfection, gene gun, lipid based transfection or other conventional techniques. Methods and conditions for culturing the resulting transfected cells and for recovering the expressed polypeptides are known to those skilled in the art, and can be varied or optimized depending upon the specific expression vector and mammalian host cell employed, based upon the present description.

### 7.11.2. Cells

The disclosure also provides host cells comprising a nucleic acid of the disclosure.

In one embodiment, the host cells are genetically engineered to comprise one or more nucleic acids described herein.

In one embodiment, the host cells are genetically engineered by using an expression cassette. The phrase “expression cassette,” refers to nucleotide sequences, which are capable of affecting expression of a gene in hosts compatible with such sequences. Such cassettes can include a promoter, an open reading frame with or without introns, and a termination signal. Additional factors necessary or helpful in effecting expression can also be used, such as, for example, an inducible promoter.

The disclosure also provides host cells comprising the vectors described herein.

The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells.

### 7.12. Antibody-Drug Conjugates

The MBMs (e.g., BBMs) can be conjugated, e.g., via a linker, to a drug moiety. Such conjugates are referred to herein as antibody-drug conjugates (or “ADCs”) for convenience, notwithstanding the fact that one or more (or all) of the ABMs might be based on non-immunoglobulin scaffolds.

In certain aspects, the drug moiety exerts a cytotoxic or cytostatic activity. In one embodiment, the drug moiety is chosen from a maytansinoid, a kinesin-like protein KIF11 inhibitor, a V-ATPase (vacuolar-type H+-ATPase) inhibitor, a pro-apoptotic agent, a Bcl2 (B-cell lymphoma 2) inhibitor, an MCL1 (myeloid cell leukemia 1) inhibitor, a HSP90 (heat shock protein 90) inhibitor, an IAP (inhibitor of apoptosis) inhibitor, an mTOR (mechanistic target of rapamycin) inhibitor, a microtubule stabilizer, a microtubule destabilizer, an auristatin, a dolastatin, a MetAP (methionine aminopeptidase), a CRM1 (chromosomal maintenance 1) inhibitor, a DPPIV (dipeptidyl peptidase IV) inhibitor, a proteasome inhibitor, an inhibitor of a phosphoryl transfer reaction in mitochondria, a protein synthesis inhibitor, a kinase inhibitor, a CDK2 (cyclin-dependent kinase 2) inhibitor, a CDK9 (cyclin-dependent kinase 9) inhibitor, a kinesin inhibitor, an HDAC (histone deacetylase) inhibitor, a DNA damaging agent, a DNA alkylating agent, a DNA intercalator, a DNA minor groove binder, a RNA polymerase inhibitor, a topoisomerase inhibitor, or a DHFR (dihydrofolate reductase) inhibitor.

In one embodiment, the linker is chosen from a cleavable linker, a non-cleavable linker, a hydrophilic linker, a procharged linker, or a dicarboxylic acid based linker.

In some embodiments, the ADCs are compounds according to structural formula (I):

[D-L-XY]ₙ-Ab

or salts thereof, where each “D” represents, independently of the others, a cytotoxic and/or cytostatic agent (“drug”); each “L” represents, independently of the others, a linker; “Ab” represents a MBM described herein; each “XY” represents a linkage formed between a functional group R^{x} on the linker and a “complementary” functional group R^{y} on the antibody, and n represents the number of drugs linked to, or drug-to-antibody ratio (DAR), of the ADC.

Some embodiments of the various antibodies (Ab) that can comprise the ADCs include the various embodiments of MBMs described above.

In some embodiments of the ADCs and/or salts of structural formula (I), each D is the same and/or each L is the same.

Some embodiments of cytotoxic and/or cytostatic agents (D) and linkers (L) that can comprise the ADCs of the disclosure, as well as the number of cytotoxic and/or cytostatic agents linked to the ADCs, are described in more detail below.

### 7.12.1. Cytotoxic and/or Cytostatic Agents

The cytotoxic and/or cytostatic agents can be any agents known to inhibit the growth and/or replication of and/or kill cells, and in particular cancer and/or tumor cells. Numerous agents having cytotoxic and/or cytostatic properties are known in the literature. Non-limiting examples of classes of cytotoxic and/or cytostatic agents include, by way of example and not limitation, radionuclides, alkylating agents, topoisomerase I inhibitors, topoisomerase II inhibitors, DNA intercalating agents (e.g., groove binding agents such as minor groove binders), RNA/DNA antimetabolites, cell cycle modulators, kinase inhibitors, protein synthesis inhibitors, histone deacetylase inhibitors, mitochondria inhibitors, and antimitotic agents.

Specific non-limiting examples of agents within certain of these various classes are provided below.

Alkylatinq Agents: asaley ((L-Leucine, N-[N-acetyl-4-[bis-(2-chloroethyl)amino]-DL-phenylalanyl]-, ethylester; NSC 167780; CAS Registry No. 3577897)); AZQ ((1,4-cyclohexadiene-1,4-dicarbamic acid, 2,5-bis(1-aziridinyl)-3,6-dioxo-, diethyl ester; NSC 182986; CAS Registry No. 57998682)); BCNU ((N,N′-Bis(2-chloroethyl)-N-nitrosourea; NSC 409962; CAS Registry No. 154938)); busulfan (1,4-butanediol dimethanesulfonate; NSC 750; CAS Registry No. 55981); (carboxyphthalato)platinum (NSC 27164; CAS Registry No. 65296813); CBDCA ((cis-(1,1-cyclobutanedicarboxylato)diammineplatinum(II)); NSC 241240; CAS Registry No. 41575944)); CCNU ((N-(2-chloroethyl)-N′-cyclohexyl-N-nitrosourea; NSC 79037; CAS Registry No. 13010474)); CHIP (iproplatin; NSC 256927); chlorambucil (NSC 3088; CAS Registry No. 305033); chlorozotocin ((2-[[[(2-chloroethyl) nitrosoamino]carbonyl]amino]-2-deoxy-D-glucopyranose; NSC 178248; CAS Registry No. 54749905)); cis-platinum (cisplatin; NSC 119875; CAS Registry No. 15663271); clomesone (NSC 338947; CAS Registry No. 88343720); cyanomorpholinodoxorubicin (NCS 357704; CAS Registry No. 88254073); cyclodisone (NSC 348948; CAS Registry No. 99591738); dianhydrogalactitol (5,6-diepoxydulcitol; NSC 132313; CAS Registry No. 23261203); fluorodopan ((5-[(2-chloroethyl)-(2-fluoroethyl)amino]-6-methyl-uracil; NSC 73754; CAS Registry No. 834913); hepsulfam (NSC 329680; CAS Registry No. 96892578); hycanthone (NSC 142982; CAS Registry No. 23255938); melphalan (NSC 8806; CAS Registry No. 3223072); methyl CCNU ((1-(2-chloroethyl)-3-(trans-4-methylcyclohexane)-1-nitrosourea; NSC 95441; 13909096); mitomycin C (NSC 26980; CAS Registry No. 50077); mitozolamide (NSC 353451; CAS Registry No. 85622953); nitrogen mustard ((bis(2-chloroethyl)methylamine hydrochloride; NSC 762; CAS Registry No. 55867); PCNU ((1-(2-chloroethyl)-3-(2,6-dioxo-3-piperidyl)-1-nitrosourea; NSC 95466; CAS Registry No. 13909029)); piperazine alkylator ((1-(2-chloroethyl)-4-(3-chloropropyl)-piperazine dihydrochloride; NSC 344007)); piperazinedione (NSC 135758; CAS Registry No. 41109802); pipobroman ((N,N-bis(3-bromopropionyl) piperazine; NSC 25154; CAS Registry No. 54911)); porfiromycin (N-methylmitomycin C; NSC 56410; CAS Registry No. 801525); spirohydantoin mustard (NSC 172112; CAS Registry No. 56605164); teroxirone (triglycidylisocyanurate; NSC 296934; CAS Registry No. 2451629); tetraplatin (NSC 363812; CAS Registry No. 62816982); thio-tepa (N,N′,N″-tri-1,2-ethanediylthio phosphoramide; NSC 6396; CAS Registry No. 52244); triethylenemelamine (NSC 9706; CAS Registry No. 51183); uracil nitrogen mustard (desmethyldopan; NSC 34462; CAS Registry No. 66751); Yoshi-864 ((bis(3-mesyloxy propyl)amine hydrochloride; NSC 102627; CAS Registry No. 3458228).

Topoisomerase I Inhibitors: camptothecin (NSC 94600; CAS Registry No. 7689-03-4); various camptothecin derivatives and analogs (for example, NSC 100880, NSC 603071, NSC 107124, NSC 643833, NSC 629971, NSC 295500, NSC 249910, NSC 606985, NSC 74028, NSC 176323, NSC 295501, NSC 606172, NSC 606173, NSC 610458, NSC 618939, NSC 610457, NSC 610459, NSC 606499, NSC 610456, NSC 364830, and NSC 606497); morpholinisoxorubicin (NSC 354646; CAS Registry No. 89196043); SN-38 (NSC 673596; CAS Registry No. 86639-52-3).

Topoisomerase II Inhibitors: doxorubicin (NSC 123127; CAS Registry No. 25316409); amonafide (benzisoquinolinedione; NSC 308847; CAS Registry No. 69408817); m-AMSA ((4′-(9-acridinylamino)-3′-methoxymethanesulfonanilide; NSC 249992; CAS Registry No. 51264143)); anthrapyrazole derivative ((NSC 355644); etoposide (VP-16; NSC 141540; CAS Registry No. 33419420); pyrazoloacridine ((pyrazolo[3,4,5-kl]acridine-2(6H)-propanamine, 9-methoxy-N, N-dimethyl-5-nitro-, monomethanesulfonate; NSC 366140; CAS Registry No. 99009219); bisantrene hydrochloride (NSC 337766; CAS Registry No. 71439684); daunorubicin (NSC 821151; CAS Registry No. 23541506); deoxydoxorubicin (NSC 267469; CAS Registry No. 63950061); mitoxantrone (NSC 301739; CAS Registry No. 70476823); menogaril (NSC 269148; CAS Registry No. 71628961); N,N-dibenzyl daunomycin (NSC 268242; CAS Registry No. 70878512); oxanthrazole (NSC 349174; CAS Registry No. 105118125); rubidazone (NSC 164011; CAS Registry No. 36508711); teniposide (VM-26; NSC 122819; CAS Registry No. 29767202).

DNA Intercalating Agents: anthramycin (CAS Registry No. 4803274); chicamycin A (CAS Registry No. 89675376); tomaymycin (CAS Registry No. 35050556); DC-81 (CAS Registry No. 81307246); sibiromycin (CAS Registry No. 12684332); pyrrolobenzodiazepine derivative (CAS Registry No. 945490095); SGD-1882 ((S)-2-(4-aminophenyl)-7-methoxy-8-(3-4(S)-7-methoxy-2-(4-methoxyphenyl)-5-oxo-5,11a-dihydro-1H-benzo[e]pyrrolo[1,2-a][1,4]diazepin-8-yl)oxy)propox-y)-1H-benzo[e]pyrrolo[1,2-a][1,4]diazepin-5(11aH)-one); SG2000 (SJG-136; (11aS,11a′S)-8,8′-(propane-1,3-diylbis(oxy))bis(7-methoxy-2-methylene-2,3-dihydro-1H-benzo[e]pyrrolo[1,2-a][1,4]diazepin-5(11aH)-one); NSC 694501; CAS Registry No. 232931576).

RNA/DNA Antimetabolites: L-alanosine (NSC 153353; CAS Registry No. 59163416); 5-azacytidine (NSC 102816; CAS Registry No. 320672); 5-fluorouracil (NSC 19893; CAS Registry No. 51218); acivicin (NSC 163501; CAS Registry No. 42228922); aminopterin derivative N42-chloro-5-[[(2,4-diamino-5-methyl-6-quinazolinyl)methyl]amino]benzoyl-]L-aspartic acid (NSC 132483); aminopterin derivative N44-[[(2,4-diamino-5-ethyl-6-quinazolinyl)methyl]amino]benzoyl]L-asparti-c acid (NSC 184692); aminopterin derivative N42-chloro-4-[[(2,4-diamino-6-pteridinyl)methyl]amino]benzoyl]L-aspartic acid monohydrate (NSC 134033); an antifo ((N^{α}-(4-amino-4-deoxypteroyl)-N⁷-hemiphthaloyl-L-ornithin-e; NSC 623017)); Baker's soluble antifol (NSC 139105; CAS Registry No. 41191042); dichlorallyl lawsone ((2-(3,3-dichloroallyl)-3-hydroxy-1,4-naphthoquinone; NSC 126771; CAS Registry No. 36417160); brequinar (NSC 368390; CAS Registry No. 96201886); ftorafur ((pro-drug; 5-fluoro-1-(tetrahydro-2-furyl)-uracil; NSC 148958; CAS Registry No. 37076689); 5,6-dihydro-5-azacytidine (NSC 264880; CAS Registry No. 62402317); methotrexate (NSC 740; CAS Registry No. 59052); methotrexate derivative (N-[[4-[[(2,4-diamino-6-pteridinyl)methyl]methylamino]-1-naphthalenyl]car-bonyl]L-glutamic acid; NSC 174121); PALA ((N-(phosphonoacetyl)-L-aspartate; NSC 224131; CAS Registry No. 603425565); pyrazofurin (NSC 143095; CAS Registry No. 30868305); trimetrexate (NSC 352122; CAS Registry No. 82952645).

DNA Antimetabolites: 3-HP (NSC 95678; CAS Registry No. 3814797); 2′-deoxy-5-fluorouridine (NSC 27640; CAS Registry No. 50919); 5-HP (NSC 107392; CAS Registry No. 19494894); α-TGDR (α-2′-deoxy-6-thioguanosine; NSC 71851 CAS Registry No. 2133815); aphidicolin glycinate (NSC 303812; CAS Registry No. 92802822); ara C (cytosine arabinoside; NSC 63878; CAS Registry No. 69749); 5-aza-2′-deoxycytidine (NSC 127716; CAS Registry No. 2353335); β-TGDR (β-2′-deoxy-6-thioguanosine; NSC 71261; CAS Registry No. 789617); cyclocytidine (NSC 145668; CAS Registry No. 10212256); guanazole (NSC 1895; CAS Registry No. 1455772); hydroxyurea (NSC 32065; CAS Registry No. 127071); inosine glycodialdehyde (NSC 118994; CAS Registry No. 23590990); macbecin II (NSC 330500; CAS Registry No. 73341738); pyrazoloimidazole (NSC 51143; CAS Registry No. 6714290); thioguanine (NSC 752; CAS Registry No. 154427); thiopurine (NSC 755; CAS Registry No. 50442).

Cell Cycle Modulators: silibinin (CAS Registry No. 22888-70-6); epigallocatechin gallate (EGCG; CAS Registry No. 989515); procyanidin derivatives (e.g., procyanidin A1 [CAS Registry No. 103883030], procyanidin B1 [CAS Registry No. 20315257], procyanidin B4 [CAS Registry No. 29106512], arecatannin B1 [CAS Registry No. 79763283]); isoflavones (e.g., genistein [4′,5,7-trihydroxyisoflavone; CAS Registry No. 446720], daidzein [4′,7-dihydroxyisoflavone, CAS Registry No. 486668]; indole-3-carbinol (CAS Registry No. 700061); quercetin (NSC 9219; CAS Registry No. 117395); estramustine (NSC 89201; CAS Registry No. 2998574); nocodazole (CAS Registry No. 31430189); podophyllotoxin (CAS Registry No. 518285); vinorelbine tartrate (NSC 608210; CAS Registry No. 125317397); cryptophycin (NSC 667642; CAS Registry No. 124689652).

Kinase Inhibitors: afatinib (CAS Registry No. 850140726); axitinib (CAS Registry No. 319460850); ARRY-438162 (binimetinib) (CAS Registry No. 606143899); bosutinib (CAS Registry No. 380843754); cabozantinib (CAS Registry No. 1140909483); ceritinib (CAS Registry No. 1032900256); crizotinib (CAS Registry No. 877399525); dabrafenib (CAS Registry No. 1195765457); dasatinib (NSC 732517; CAS Registry No. 302962498); erlotinib (NSC 718781; CAS Registry No. 183319699); everolimus (NSC 733504; CAS Registry No. 159351696); fostamatinib (NSC 745942; CAS Registry No. 901119355); gefitinib (NSC 715055; CAS Registry No. 184475352); ibrutinib (CAS Registry No. 936563961); imatinib (NSC 716051; CAS Registry No. 220127571); lapatinib (CAS Registry No. 388082788); lenvatinib (CAS Registry No. 857890392); mubritinib (CAS 366017096); nilotinib (CAS Registry No. 923288953); nintedanib (CAS Registry No. 656247175); palbociclib (CAS Registry No. 571190302); pazopanib (NSC 737754; CAS Registry No. 635702646); pegaptanib (CAS Registry No. 222716861); ponatinib (CAS Registry No. 1114544318); rapamycin (NSC 226080; CAS Registry No. 53123889); regorafenib (CAS Registry No. 755037037); AP 23573 (ridaforolimus) (CAS Registry No. 572924540); INCB018424 (ruxolitinib) (CAS Registry No. 1092939177); ARRY-142886 (selumetinib) (NSC 741078; CAS Registry No. 606143-52-6); sirolimus (NSC 226080; CAS Registry No. 53123889); sorafenib (NSC 724772; CAS Registry No. 475207591); sunitinib (NSC 736511; CAS Registry No. 341031547); tofacitinib (CAS Registry No. 477600752); temsirolimus (NSC 683864; CAS Registry No. 163635043); trametinib (CAS Registry No. 871700173); vandetanib (CAS Registry No. 443913733); vemurafenib (CAS Registry No. 918504651); SU6656 (CAS Registry No. 330161870); CEP-701 (lesaurtinib) (CAS Registry No. 111358884); XL019 (CAS Registry No. 945755566); PD-325901 (CAS Registry No. 391210109); PD-98059 (CAS Registry No. 167869218); ATP-competitive TORC1/TORC2 inhibitors including PI-103 (CAS Registry No. 371935749), PP242 (CAS Registry No. 1092351671), PP30 (CAS Registry No. 1092788094), Torin 1 (CAS Registry No. 1222998368), LY294002 (CAS Registry No. 154447366), XL-147 (CAS Registry No. 934526893), CAL-120 (CAS Registry No. 870281348), ETP-45658 (CAS Registry No. 1198357797), PX 866 (CAS Registry No. 502632668), GDC-0941 (CAS Registry No. 957054307), BGT226 (CAS Registry No. 1245537681), BEZ235 (CAS Registry No. 915019657), XL-765 (CAS Registry No. 934493762).

Protein Synthesis Inhibitors: acriflavine (CAS Registry No. 65589700); amikacin (NSC 177001; CAS Registry No. 39831555); arbekacin (CAS Registry No. 51025855); astromicin (CAS Registry No. 55779061); azithromycin (NSC 643732; CAS Registry No. 83905015); bekanamycin (CAS Registry No. 4696768); chlortetracycline (NSC 13252; CAS Registry No. 64722); clarithromycin (NSC 643733; CAS Registry No. 81103119); clindamycin (CAS Registry No. 18323449); clomocycline (CAS Registry No. 1181540); cycloheximide (CAS Registry No. 66819); dactinomycin (NSC 3053; CAS Registry No. 50760); dalfopristin (CAS Registry No. 112362502); demeclocycline (CAS Registry No. 127333); dibekacin (CAS Registry No. 34493986); dihydrostreptomycin (CAS Registry No. 128461); dirithromycin (CAS Registry No. 62013041); doxycycline (CAS Registry No. 17086281); emetine (NSC 33669; CAS Registry No. 483181); erythromycin (NSC 55929; CAS Registry No. 114078); flurithromycin (CAS Registry No. 83664208); framycetin (neomycin B; CAS Registry No. 119040); gentamycin (NSC 82261; CAS Registry No. 1403663); glycylcyclines, such as tigecycline (CAS Registry No. 220620097); hygromycin B (CAS Registry No. 31282049); isepamicin (CAS Registry No. 67814760); josamycin (NSC 122223; CAS Registry No. 16846245); kanamycin (CAS Registry No. 8063078); ketolides such as telithromycin (CAS Registry No. 191114484), cethromycin (CAS Registry No. 205110481), and solithromycin (CAS Registry No. 760981837); lincomycin (CAS Registry No. 154212); lymecycline (CAS Registry No. 992212); meclocycline (NSC 78502; CAS Registry No. 2013583); metacycline (rondomycin; NSC 356463; CAS Registry No. 914001); midecamycin (CAS Registry No. 35457808); minocycline (NSC 141993; CAS Registry No. 10118908); miocamycin (CAS Registry No. 55881077); neomycin (CAS Registry No. 119040); netilmicin (CAS Registry No. 56391561); oleandomycin (CAS Registry No. 3922905); oxazolidinones, such as eperezolid (CAS Registry No. 165800044), linezolid (CAS Registry No. 165800033), posizolid (CAS Registry No. 252260029), radezolid (CAS Registry No. 869884786), ranbezolid (CAS Registry No. 392659380), sutezolid (CAS Registry No. 168828588), tedizolid (CAS Registry No. 856867555); oxytetracycline (NSC 9169; CAS Registry No. 2058460); paromomycin (CAS Registry No. 7542372); penimepicycline (CAS Registry No. 4599604); peptidyl transferase inhibitors, e.g., chloramphenicol (NSC 3069; CAS Registry No. 56757) and derivatives such as azidamfenicol (CAS Registry No. 13838089), florfenicol (CAS Registry No. 73231342), and thiamphenicol (CAS Registry No. 15318453), and pleuromutilins such as retapamulin (CAS Registry No. 224452668), tiamulin (CAS Registry No. 55297955), valnemulin (CAS Registry No. 101312929); pirlimycin (CAS Registry No. 79548735); puromycin (NSC 3055; CAS Registry No. 53792); quinupristin (CAS Registry No. 120138503); ribostamycin (CAS Registry No. 53797356); rokitamycin (CAS Registry No. 74014510); rolitetracycline (CAS Registry No. 751973); roxithromycin (CAS Registry No. 80214831); sisomicin (CAS Registry No. 32385118); spectinomycin (CAS Registry No. 1695778); spiramycin (CAS Registry No. 8025818); streptogramins such as pristinamycin (CAS Registry No. 270076603), quinupristin/dalfopristin (CAS Registry No. 126602899), and virginiamycin (CAS Registry No. 11006761); streptomycin (CAS Registry No. 57921); tetracycline (NSC 108579; CAS Registry No. 60548); tobramycin (CAS Registry No. 32986564); troleandomycin (CAS Registry No. 2751099); tylosin (CAS Registry No. 1401690); verdamicin (CAS Registry No. 49863481).

Histone Deacetylase Inhibitors: abexinostat (CAS Registry No. 783355602); belinostat (NSC 726630; CAS Registry No. 414864009); chidamide (CAS Registry No. 743420022); entinostat (CAS Registry No. 209783802); givinostat (CAS Registry No. 732302997); mocetinostat (CAS Registry No. 726169739); panobinostat (CAS Registry No. 404950807); quisinostat (CAS Registry No. 875320299); resminostat (CAS Registry No. 864814880); romidepsin (CAS Registry No. 128517077); sulforaphane (CAS Registry No. 4478937); thioureidobutyronitrile (Kevetrin™; CAS Registry No. 6659890); valproic acid (NSC 93819; CAS Registry No. 99661); vorinostat (NSC 701852; CAS Registry No. 149647789); ACY-1215 (rocilinostat; CAS Registry No. 1316214524); CUDC-101 (CAS Registry No. 1012054599); CHR-2845 (tefinostat; CAS Registry No. 914382608); CHR-3996 (CAS Registry No. 1235859138); 4SC-202 (CAS Registry No. 910462430); CG200745 (CAS Registry No. 936221339); SB939 (pracinostat; CAS Registry No. 929016966).

Mitochondria Inhibitors: pancratistatin (NSC 349156; CAS Registry No. 96281311); rhodamine-123 (CAS Registry No. 63669709); edelfosine (NSC 324368; CAS Registry No. 70641519); d-alpha-tocopherol succinate (NSC 173849; CAS Registry No. 4345033); compound 11β (CAS Registry No. 865070377); aspirin (NSC 406186; CAS Registry No. 50782); ellipticine (CAS Registry No. 519233); berberine (CAS Registry No. 633658); cerulenin (CAS Registry No. 17397896); GX015-070 (Obatoclax®; 1H-Indole, 2-(2-((3,5-dimethyl-1H-pyrrol-2-yl)methylene)-3-methoxy-2H-pyrrol-5-yl)-; NSC 729280; CAS Registry No. 803712676); celastrol (tripterine; CAS Registry No. 34157830); metformin (NSC 91485; CAS Registry No. 1115704); Brilliant green (NSC 5011; CAS Registry No. 633034); ME-344 (CAS Registry No. 1374524556).

Antimitotic Agents: allocolchicine (NSC 406042); auristatins, such as MMAE (monomethyl auristatin E; CAS Registry No. 474645-27-7) and MMAF (monomethyl auristatin F; CAS Registry No. 745017-94-1; halichondrin B (NSC 609395); colchicine (NSC 757; CAS Registry No. 64868); cholchicine derivative (N-benzoyl-deacetyl benzamide; NSC 33410; CAS Registry No. 63989753); dolastatin 10 (NSC 376128; CAS Registry No 110417-88-4); maytansine (NSC 153858; CAS Registry No. 35846-53-8); rhozoxin (NSC 332598; CAS Registry No. 90996546); taxol (NSC 125973; CAS Registry No. 33069624); taxol derivative ((2′-N-[3-(dimethylamino)propyl]glutaramate taxol; NSC 608832); thiocolchicine (3-demethylthiocolchicine; NSC 361792); trityl cysteine (NSC 49842; CAS Registry No. 2799077); vinblastine sulfate (NSC 49842; CAS Registry No. 143679); vincristine sulfate (NSC 67574; CAS Registry No. 2068782).

Any of these agents that include or that can be modified to include a site of attachment to a MBM can be included in the ADCs disclosed herein.

In some embodiments, the cytotoxic and/or cytostatic agent is an antimitotic agent.

In some embodiments, the cytotoxic and/or cytostatic agent is an auristatin, for example, monomethyl auristatin E (“MMAE:) or monomethyl auristatin F (“MMAF”).

### 7.12.2. ADC Linkers

In the ADCs of the disclosure, the cytotoxic and/or cytostatic agents are linked to the MBM by way of ADC linkers. The ADC linker linking a cytotoxic and/or cytostatic agent to the MBM of an ADC can be short, long, hydrophobic, hydrophilic, flexible or rigid, or can be composed of segments that each independently have one or more of the above-mentioned properties such that the linker can include segments having different properties. The linkers can be polyvalent such that they covalently link more than one agent to a single site on the MBM, or monovalent such that covalently they link a single agent to a single site on the MBM.

As will be appreciated by a skilled artisan, the ADC linkers link cytotoxic and/or cytostatic agents to the MBM by forming a covalent linkage to the cytotoxic and/or cytostatic agent at one location and a covalent linkage to the MBM at another. The covalent linkages are formed by reaction between functional groups on the ADC linker and functional groups on the agents and MBM. As used herein, the expression “ADC linker” is intended to include (i) unconjugated forms of the ADC linker that include a functional group capable of covalently linking the ADC linker to a cytotoxic and/or cytostatic agent and a functional group capable of covalently linking the ADC linker to a MBM; (ii) partially conjugated forms of the ADC linker that include a functional group capable of covalently linking the ADC linker to a MBM and that is covalently linked to a cytotoxic and/or cytostatic agent, or vice versa; and (iii) fully conjugated forms of the ADC linker that are covalently linked to both a cytotoxic and/or cytostatic agent and a MBM. In some embodiments of ADC linkers and ADCs of the disclosure, as well as synthons used to conjugate linker-agents to MBMs, moieties comprising the functional groups on the ADC linker and covalent linkages formed between the ADC linker and MBM are specifically illustrated as Rₓ and XY, respectively.

The ADC linkers are, but need not be, chemically stable to conditions outside the cell, and can be designed to cleave, immolate and/or otherwise specifically degrade inside the cell. Alternatively, ADC linkers that are not designed to specifically cleave or degrade inside the cell can be used. Choice of stable versus unsTable 8DC linker can depend upon the toxicity of the cytotoxic and/or cytostatic agent. For agents that are toxic to normal cells, stable linkers can be used. Agents that are selective or targeted and have lower toxicity to normal cells can be utilized, as chemical stability of the ADC linker to the extracellular milieu is less important. A wide variety of ADC linkers useful for linking drugs to MBMs in the context of ADCs are known. Any of these ADC linkers, as well as other ADC linkers, can be used to link the cytotoxic and/or cytostatic agents to the MBM of the ADCs of the disclosure.

Exemplary polyvalent ADC linkers that can be used to link many cytotoxic and/or cytostatic agents to a single MBM molecule are described, for example, in WO 2009/073445; WO 2010/068795; WO 2010/138719; WO 2011/120053; WO 2011/171020; WO 2013/096901; WO 2014/008375; WO 2014/093379; WO 2014/093394; WO 2014/093640. For example, the Fleximer linker technology developed by Mersana et al. has the potential to enable high-DAR ADCs with good physicochemical properties. As shown below, the Mersana technology is based on incorporating drug molecules into a solubilizing poly-acetal backbone via a sequence of ester bonds. The methodology renders highly-loaded ADCs (DAR up to 20) while maintaining good physicochemical properties.

Additional examples of dendritic type linkers can be found in US 2006/116422; US 2005/271615; de Groot et al., 2003, Angew. Chem. Int. Ed. 42:4490-4494; Amir et al., 2003, Angew. Chem. Int. Ed. 42:4494-4499; Shamis et al., 2004, J. Am. Chem. Soc. 126:1726-1731; Sun et al., 2002, Bioorganic & Medicinal Chemistry Letters 12:2213-2215; Sun et al., 2003, Bioorganic & Medicinal Chemistry 11:1761-1768; King et al., 2002, Tetrahedron Letters 43:1987-1990.

Exemplary monovalent ADC linkers that can be used are described, for example, in Nolting, 2013, Antibody-Drug Conjugates, Methods in Molecular Biology 1045:71-100; Kitson et al., 2013, CROs—MOs—Chemica—ggi—Chemistry Today 31(4):30-38; Ducry et al., 2010, Bioconjugate Chem. 21:5-13; Zhao et al., 2011, J. Med. Chem. 54:3606-3623; U.S. Pat. Nos. 7,223,837; 8,568,728; 8,535,678; and WO2004010957.

By way of example and not limitation, some cleavable and noncleavable ADC linkers that can be included in the ADCs are described below.

### 7.12.2.1. Cleavable ADC Linkers

In certain embodiments, the ADC linker selected is cleavable in vivo. Cleavable ADC linkers can include chemically or enzymatically unstable or degradable linkages. Cleavable ADC linkers generally rely on processes inside the cell to liberate the drug, such as reduction in the cytoplasm, exposure to acidic conditions in the lysosome, or cleavage by specific proteases or other enzymes within the cell. Cleavable ADC linkers generally incorporate one or more chemical bonds that are either chemically or enzymatically cleavable while the remainder of the ADC linker is noncleavable. In certain embodiments, an ADC linker comprises a chemically labile group such as hydrazone and/or disulfide groups. Linkers comprising chemically labile groups exploit differential properties between the plasma and some cytoplasmic compartments. The intracellular conditions to facilitate drug release for hydrazone containing ADC linkers are the acidic environment of endosomes and lysosomes, while the disulfide containing ADC linkers are reduced in the cytosol, which contains high thiol concentrations, e.g., glutathione. In certain embodiments, the plasma stability of an ADC linker comprising a chemically labile group can be increased by introducing steric hindrance using substituents near the chemically labile group.

Acid-labile groups, such as hydrazone, remain intact during systemic circulation in the blood's neutral pH environment (pH 7.3-7.5) and undergo hydrolysis and release the drug once the ADC is internalized into mildly acidic endosomal (pH 5.0-6.5) and lysosomal (pH 4.5-5.0) compartments of the cell. This pH dependent release mechanism has been associated with nonspecific release of the drug. To increase the stability of the hydrazone group of the ADC linker, the ADC linker can be varied by chemical modification, e.g., substitution, allowing tuning to achieve more efficient release in the lysosome with a minimized loss in circulation.

Hydrazone-containing ADC linkers can contain additional cleavage sites, such as additional acid-labile cleavage sites and/or enzymatically labile cleavage sites. ADCs including exemplary hydrazone-containing ADC linkers include the following structures:

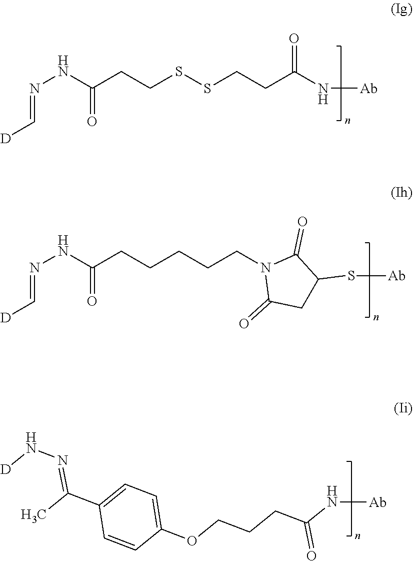

where D and Ab represent the cytotoxic and/or cytostatic agent (drug) and Ab, respectively, and n represents the number of drug-ADC linkers linked to the MBM. In certain ADC linkers such as linker (Ig), the ADC linker comprises two cleavable groups—a disulfide and a hydrazone moiety. For such ADC linkers, effective release of the unmodified free drug requires acidic pH or disulfide reduction and acidic pH. Linkers such as (lh) and (li) have been shown to be effective with a single hydrazone cleavage site.

Additional ADC linkers which remain intact during systemic circulation and undergo hydrolysis and release the drug when the ADC is internalized into acidic cellular compartments include carbonates. Such ADC linkers can be useful in cases where the cytotoxic and/or cytostatic agent can be covalently attached through an oxygen.

Other acid-labile groups that can be included in ADC linkers include cis-aconityl-containing ADC linkers. cis-Aconityl chemistry uses a carboxylic acid juxtaposed to an amide bond to accelerate amide hydrolysis under acidic conditions.

Cleavable ADC linkers can also include a disulfide group. Disulfides are thermodynamically sTable 8t physiological pH and are designed to release the drug upon internalization inside cells, where the cytosol provides a significantly more reducing environment compared to the extracellular environment. Scission of disulfide bonds generally requires the presence of a cytoplasmic thiol cofactor, such as (reduced) glutathione (GSH), such that disulfide-containing ADC linkers are reasonably stable in circulation, selectively releasing the drug in the cytosol. The intracellular enzyme protein disulfide isomerase, or similar enzymes capable of cleaving disulfide bonds, can also contribute to the preferential cleavage of disulfide bonds inside cells. GSH is reported to be present in cells in the concentration range of 0.5-10 mM compared with a significantly lower concentration of GSH or cysteine, the most abundant low-molecular weight thiol, in circulation at approximately 5 Tumor cells, where irregular blood flow leads to a hypoxic state, result in enhanced activity of reductive enzymes and therefore even higher glutathione concentrations. In certain embodiments, the in vivo stability of a disulfide-containing ADC linker can be enhanced by chemical modification of the ADC linker, e.g., use of steric hindrance adjacent to the disulfide bond.

ADCs including exemplary disulfide-containing ADC linkers include the following structures:

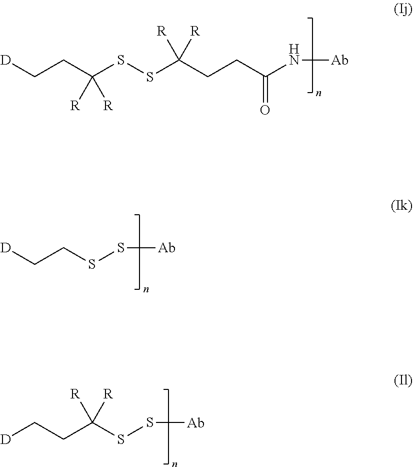

where D and Ab represent the drug and MBM, respectively, n represents the number of drug-ADC linkers linked to the MBM and R is independently selected at each occurrence from hydrogen or alkyl, for example. In certain embodiments, increasing steric hindrance adjacent to the disulfide bond increases the stability of the ADC linker. Structures such as (ID and (II) show increased in vivo stability when one or more R groups is selected from a lower alkyl such as methyl.

Another type of cleavable ADC linker that can be used is an ADC linker that is specifically cleaved by an enzyme. Such ADC linkers are typically peptide-based or include peptidic regions that act as substrates for enzymes. Peptide based ADC linkers tend to be more stable in plasma and extracellular milieu than chemically labile ADC linkers. Peptide bonds generally have good serum stability, as lysosomal proteolytic enzymes have very low activity in blood due to endogenous inhibitors and the unfavorably high pH value of blood compared to lysosomes. Release of a drug from a MBM occurs specifically due to the action of lysosomal proteases, e.g., cathepsin and plasmin. These proteases can be present at elevated levels in certain tumor cells.

In exemplary embodiments, the cleavable peptide is selected from tetrapeptides such as Gly-Phe-Leu-Gly, (SEQ ID NO: 1320), Ala-Leu-Ala-Leu (SEQ ID NO: 1321) or dipeptides such as Val-Cit, Val-Ala, Met-(D)Lys, Asn-(D)Lys, Val-(D)Asp, Phe-Lys, Ile-Val, Asp-Val, His-Val, NorVal-(D)Asp, Ala-(D)Asp 5, Met-Lys, Asn-Lys, Ile-Pro, Me3Lys-Pro, PhenylGly-(D)Lys, Met-(D)Lys, Asn-(D)Lys, Pro-(D)Lys, Met-(D)Lys, Asn-(D)Lys, AM Met-(D)Lys, Asn-(D)Lys, AW Met-(D)Lys, and Asn-(D)Lys. In certain embodiments, dipeptides can be selected over longer polypeptides due to hydrophobicity of the longer peptides.

A variety of dipeptide-based cleavable ADC linkers useful for linking drugs such as doxorubicin, mitomycin, camptothecin, pyrrolobenzodiazepine, tallysomycin and auristatin/auristatin family members to MBMs have been described (see, Dubowchik et al., 1998, J. Org. Chem. 67:1866-1872; Dubowchik et al., 1998, Bioorg. Med. Chem. Lett. 8(21):3341-3346; Walker et al., 2002, Bioorg. Med. Chem. Lett. 12:217-219; Walker et al., 2004, Bioorg. Med. Chem. Lett. 14:4323-4327; Sutherland et al., 2013, Blood 122: 1455-1463; and Francisco et al., 2003, Blood 102:1458-1465). All of these dipeptide ADC linkers, or modified versions of these dipeptide ADC linkers, can be used in the ADCs of the disclosure. Other dipeptide ADC linkers that can be used include those found in ADCs such as Seattle Genetics' Brentuximab Vendotin SGN-35 (Adcetris™), Seattle Genetics SGN-75 (anti-CD-70, Val-Cit-monomethyl auristatin F(MMAF), Seattle Genetics SGN-CD33A (anti-CD-33, Val-Ala-(SGD-1882)), Celldex Therapeutics glembatumumab (CDX-011) (anti-NMB, Val-Cit-monomethyl auristatin E (MMAE), and Cytogen PSMA-ADC (PSMA-ADC-1301) (anti-PSMA, Val-Cit-MMAE).

Enzymatically cleavable ADC linkers can include a self-immolative spacer to spatially separate the drug from the site of enzymatic cleavage. The direct attachment of a drug to a peptide ADC linker can result in proteolytic release of an amino acid adduct of the drug, thereby impairing its activity. The use of a self-immolative spacer allows for the elimination of the fully active, chemically unmodified drug upon amide bond hydrolysis.

One self-immolative spacer is the bifunctional para-aminobenzyl alcohol group, which is linked to the peptide through the amino group, forming an amide bond, while amine containing drugs can be attached through carbamate functionalities to the benzylic hydroxyl group of the ADC linker (PABC). The resulting prodrugs are activated upon protease-mediated cleavage, leading to a 1,6-elimination reaction releasing the unmodified drug, carbon dioxide, and remnants of the ADC linker group. The following scheme depicts the fragmentation of p-amidobenzyl ether and release of the drug:

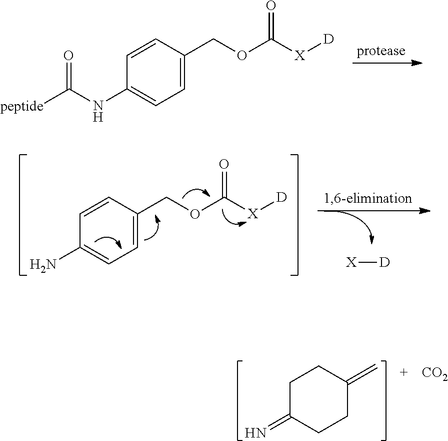

where X-D represents the unmodified drug.

Heterocyclic variants of this self-immolative group have also been described. See for example, U.S. Pat. No. 7,989,434.

In some embodiments, the enzymatically cleavable ADC linker is a β-glucuronic acid-based ADC linker. Facile release of the drug can be realized through cleavage of the β-glucuronide glycosidic bond by the lysosomal enzyme β-glucuronidase. This enzyme is present abundantly within lysosomes and is overexpressed in some tumor types, while the enzyme activity outside cells is low. β-Glucuronic acid-based ADC linkers can be used to circumvent the tendency of an ADC to undergo aggregation due to the hydrophilic nature of β-glucuronides. In some embodiments, β-glucuronic acid-based ADC linkers can be used as ADC linkers for ADCs linked to hydrophobic drugs. The following scheme depicts the release of the drug from and ADC containing a β-glucuronic acid-based ADC linker:

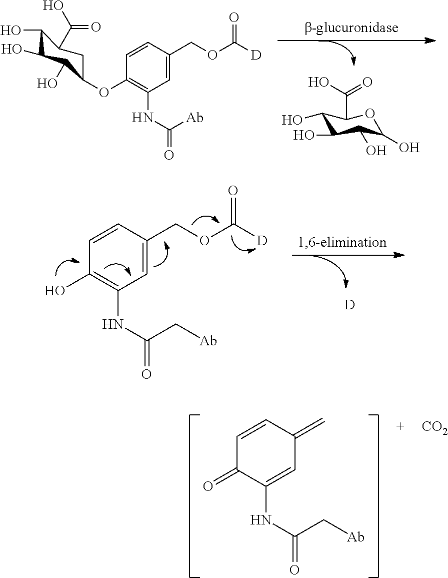

A variety of cleavable β-glucuronic acid-based ADC linkers useful for linking drugs such as auristatins, camptothecin and doxorubicin analogues, CBI minor-groove binders, and psymberin to MBMs have been described (see, Nolting, Chapter 5 “Linker Technology in Antibody-Drug Conjugates,” In: Antibody-Drug Conjugates: Methods in Molecular Biology, vol. 1045, pp. 71-100, Laurent Ducry (Ed.), Springer Science & Business Medica, LLC, 2013; Jeffrey et al., 2006, Bioconjug. Chem. 17:831-840; Jeffrey et al., 2007, Bioorg. Med. Chem. Lett. 17:2278-2280; and Jiang et al., 2005, J. Am. Chem. Soc. 127:11254-11255). All of these β-glucuronic acid-based ADC linkers can be used in the ADCs of the disclosure.

Additionally, cytotoxic and/or cytostatic agents containing a phenol group can be covalently bonded to an ADC linker through the phenolic oxygen. One such ADC linker, described in WO 2007/089149, relies on a methodology in which a diamino-ethane “SpaceLink” is used in conjunction with traditional “PABO”-based self-immolative groups to deliver phenols. The cleavage of the ADC linker is depicted schematically below, where D represents a cytotoxic and/or cytostatic agent having a phenolic hydroxyl group.

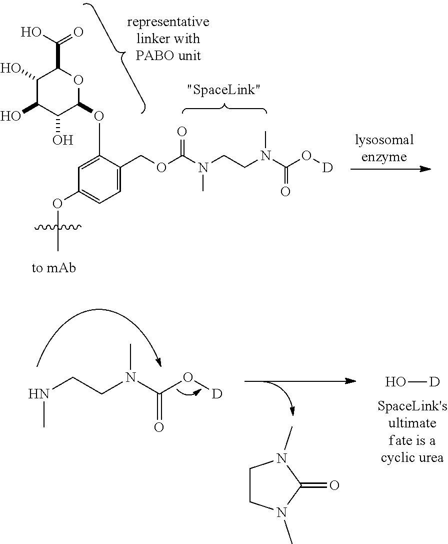

Cleavable ADC linkers can include noncleavable portions or segments, and/or cleavable segments or portions can be included in an otherwise non-cleavable ADC linker to render it cleavable. By way of example only, polyethylene glycol (PEG) and related polymers can include cleavable groups in the polymer backbone. For example, a polyethylene glycol or polymer ADC linker can include one or more cleavable groups such as a disulfide, a hydrazone or a dipeptide.

Other degradable linkages that can be included in ADC linkers include ester linkages formed by the reaction of PEG carboxylic acids or activated PEG carboxylic acids with alcohol groups on a biologically active agent, where such ester groups generally hydrolyze under physiological conditions to release the biologically active agent. Hydrolytically degradable linkages include, but are not limited to, carbonate linkages; imine linkages resulting from reaction of an amine and an aldehyde; phosphate ester linkages formed by reacting an alcohol with a phosphate group; acetal linkages that are the reaction product of an aldehyde and an alcohol; orthoester linkages that are the reaction product of a formate and an alcohol; and oligonucleotide linkages formed by a phosphoramidite group, including but not limited to, at the end of a polymer, and a 5′ hydroxyl group of an oligonucleotide.

In certain embodiments, the ADC linker comprises an enzymatically cleavable peptide moiety, for example, an ADC linker comprising structural formula (IVa) or (IVb):

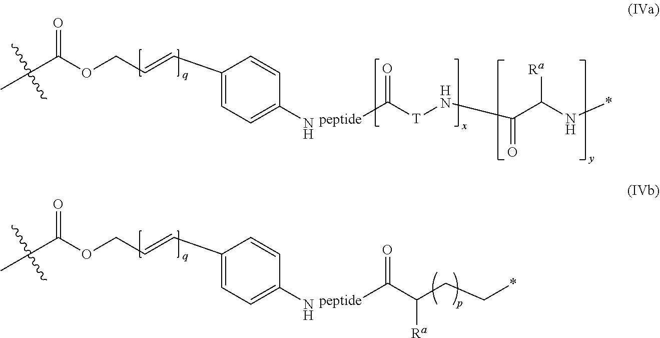

or a salt thereof, where: peptide represents a peptide (illustrated C→N and not showing the carboxy and amino “termini”) cleavable by a lysosomal enzyme; T represents a polymer comprising one or more ethylene glycol units or an alkylene chain, or combinations thereof; R^{a} is selected from hydrogen, alkyl, sulfonate and methyl sulfonate; p is an integer ranging from 0 to 5; q is 0 or 1; x is 0 or 1; y is 0 or 1; [symbol] represents the point of attachment of the ADC linker to a cytotoxic and/or cytostatic agent; and * represents the point of attachment to the remainder of the ADC linker.

In certain embodiments, the peptide is selected from a tripeptide or a dipeptide. In particular embodiments, the dipeptide is selected from: Val-Cit; Cit-Val; Ala-Ala; Ala-Cit; Cit-Ala; Asn-Cit; Cit-Asn; Cit-Cit; Val-Glu; Glu-Val; Ser-Cit; Cit-Ser; Lys-Cit; Cit-Lys; Asp-Cit; Cit-Asp; Ala-Val; Val-Ala; Phe-Lys; Val-Lys; Ala-Lys; Phe-Cit; Leu-Cit; Ile-Cit; Phe-Arg; and Trp-Cit. In certain embodiments, the dipeptide is selected from: Cit-Val; and Ala-Val.

Specific exemplary embodiments of ADC linkers according to structural formula (IVa) that can be included in the ADCs include the ADC linkers illustrated below (as illustrated, the ADC linkers include a group suitable for covalently linking the ADC linker to a MBM):

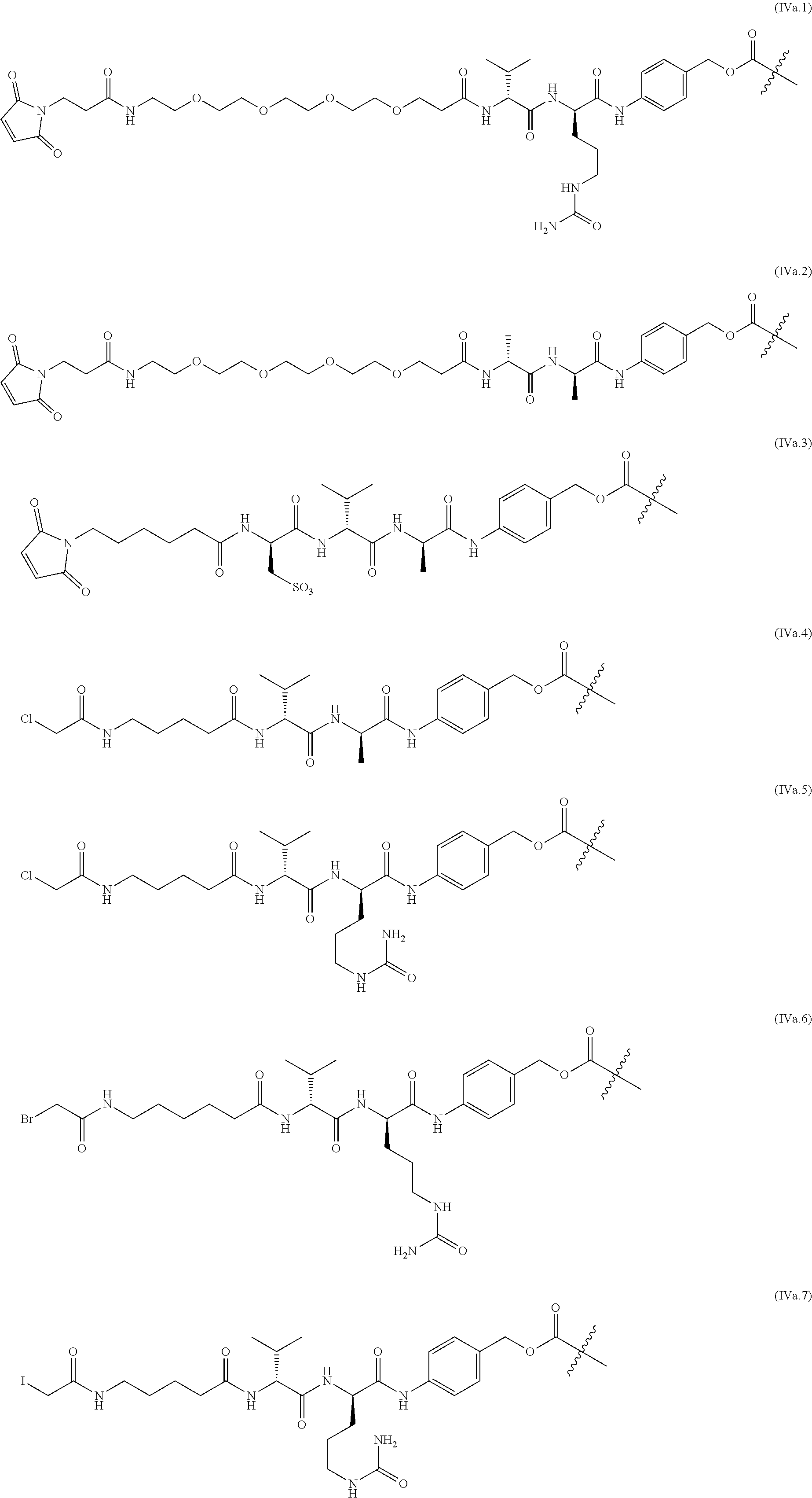

Specific exemplary embodiments of ADC linkers according to structural formula (IVb) that can be included in the ADCs include the ADC linkers illustrated below (as illustrated, the ADC linkers include a group suitable for covalently linking the ADC linker to a MBM):

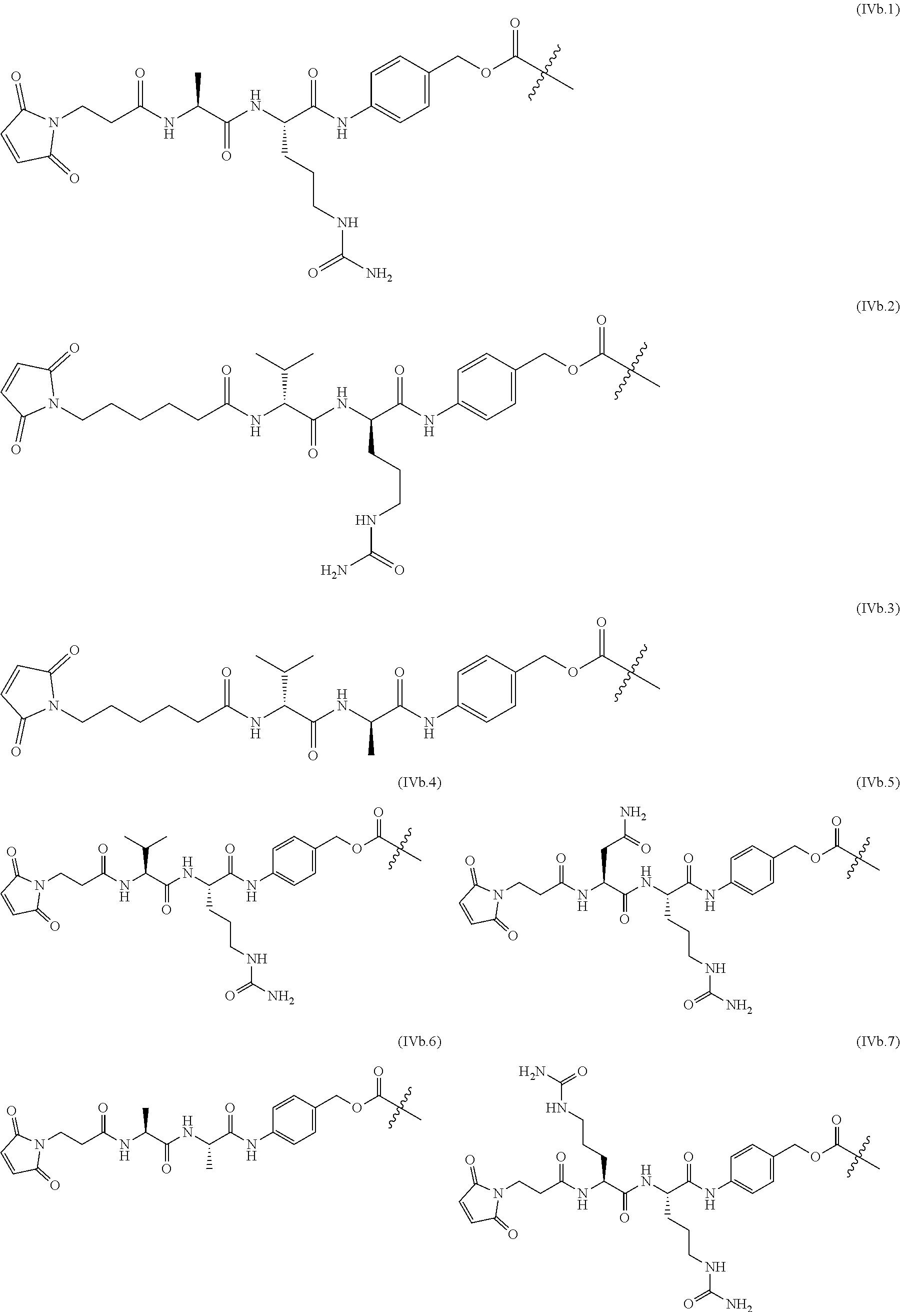

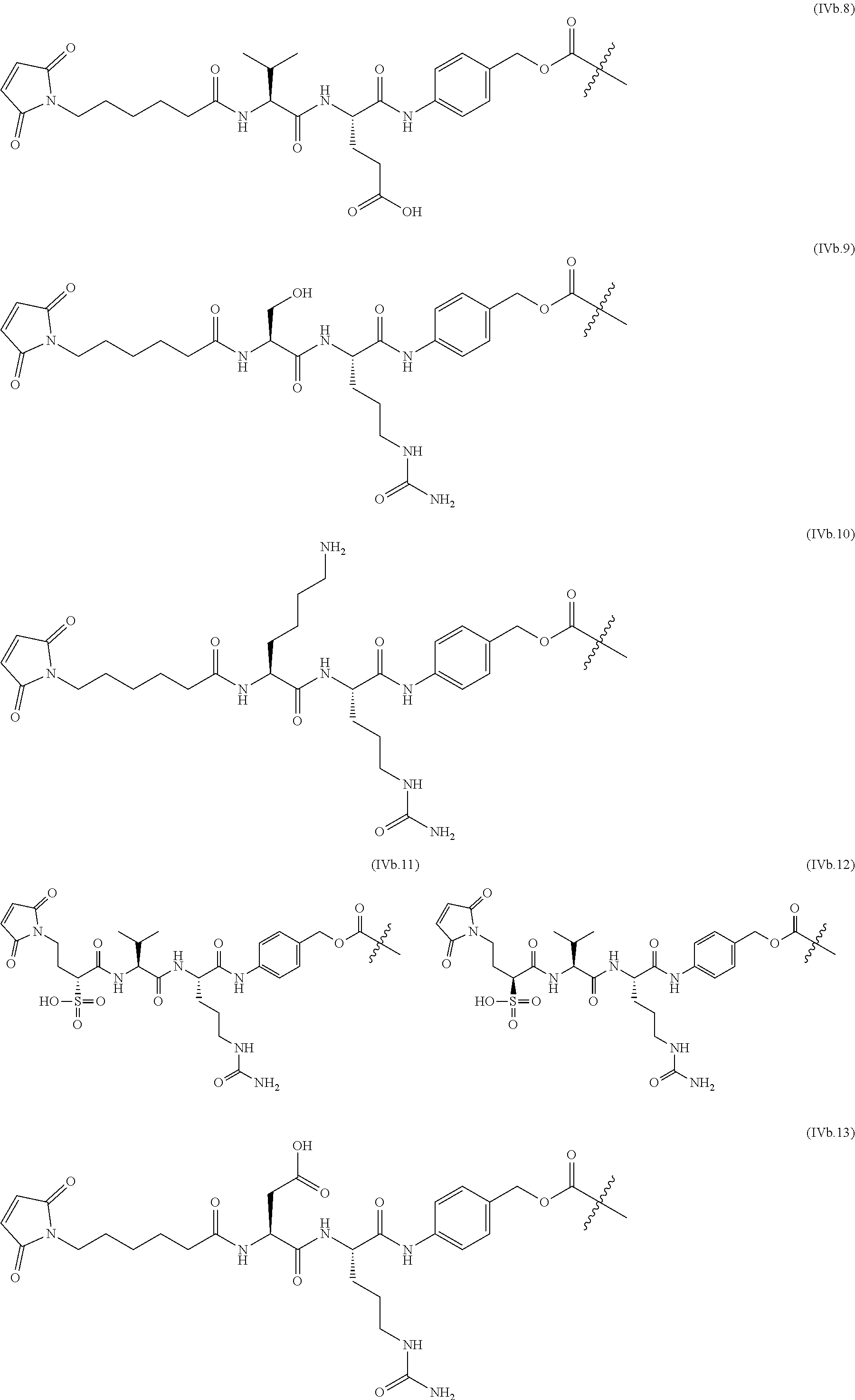

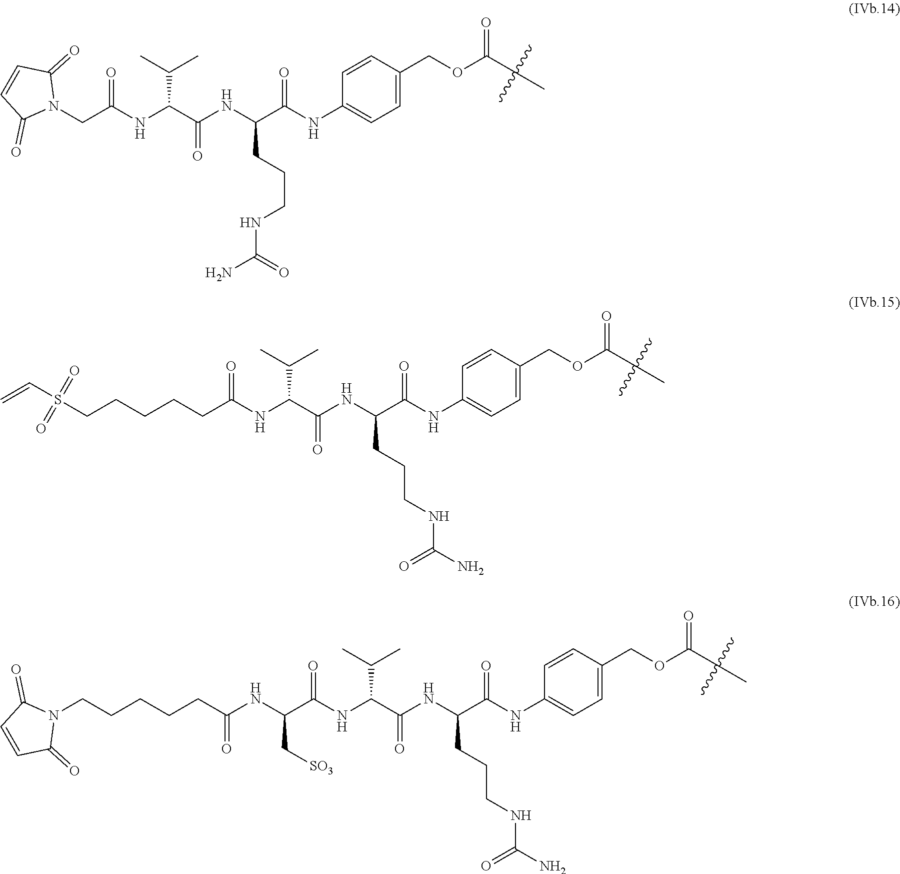

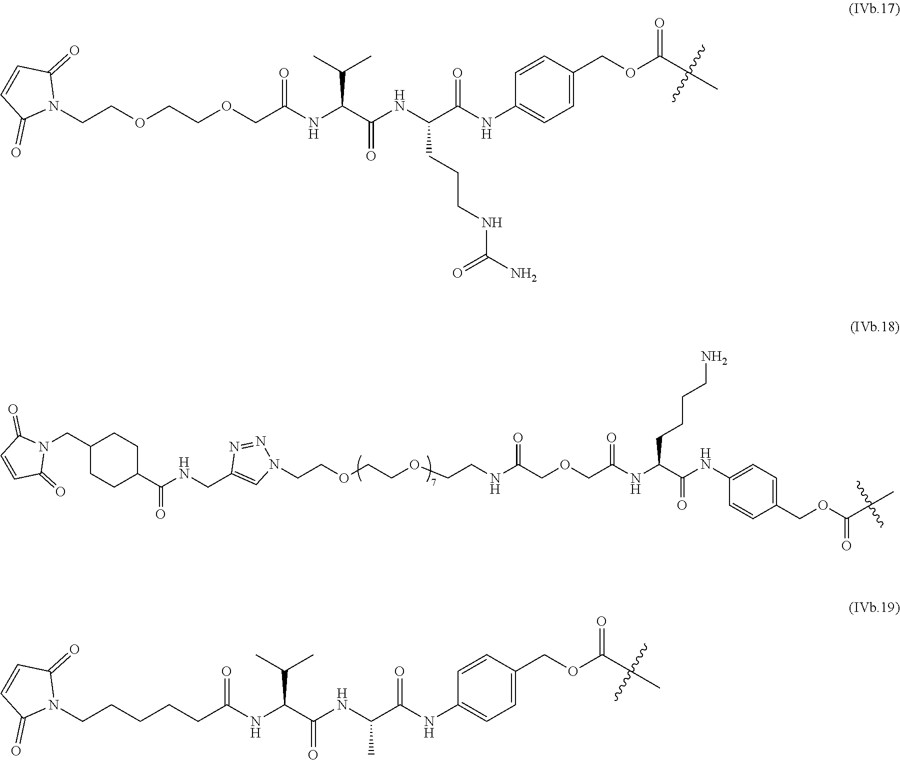

In certain embodiments, the ADC linker comprises an enzymatically cleavable peptide moiety, for example, an ADC linker comprising structural formula (IVc) or (IVd):

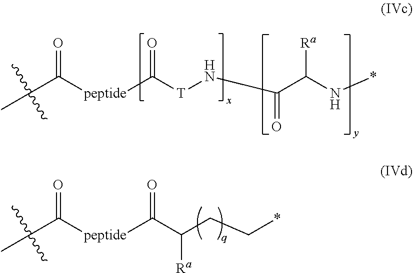

or a salt thereof, where: peptide represents a peptide (illustrated C→N and not showing the carboxy and amino “termini”) cleavable by a lysosomal enzyme; T represents a polymer comprising one or more ethylene glycol units or an alkylene chain, or combinations thereof; Ra is selected from hydrogen, alkyl, sulfonate and methyl sulfonate; p is an integer ranging from 0 to 5; q is 0 or 1; x is 0 or 1; y is 0 or 1; x [symbol] represents the point of attachment of the ADC linker to a cytotoxic and/or cytostatic agent; and * represents the point of attachment to the remainder of the ADC linker.

Specific exemplary embodiments of ADC linkers according to structural formula (IVc) that can be included in the ADCs include the ADC linkers illustrated below (as illustrated, the ADC linkers include a group suitable for covalently linking the ADC linker to a MBM):

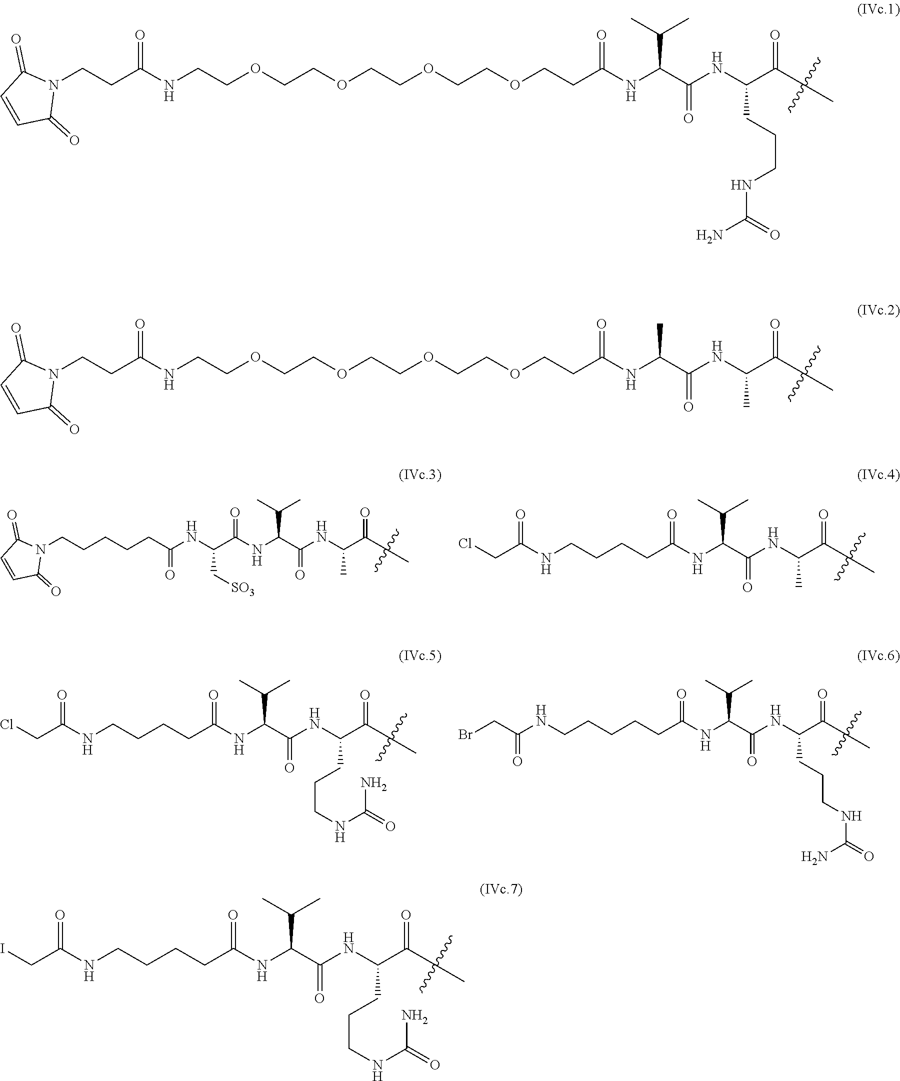

Specific exemplary embodiments of ADC linkers according to structural formula (IVd) that can be included in the ADCs include the ADC linkers illustrated below (as illustrated, the ADC linkers include a group suitable for covalently linking the ADC linker to a MBM):

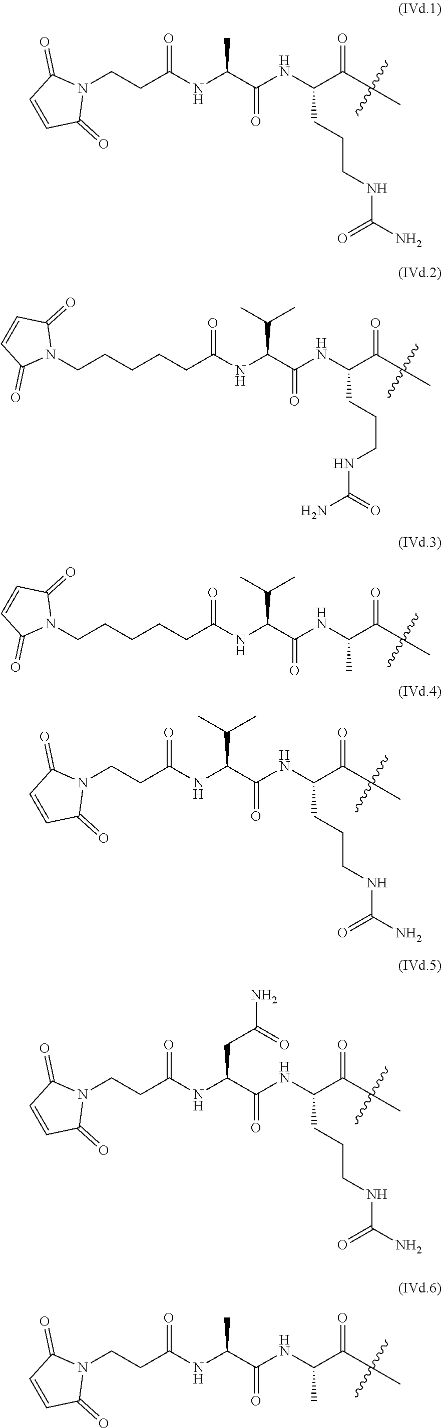 [SMILES: *C(=O)[C@H](CCCNC(N)=O)NC(=O)[C@H](C)NC(=O)CCN1C(=O)C=CC1=O ; *C(=O)[C@H](CCCNC(N)=O)NC(=O)[C@@H](NC(=O)CCCCCN1C(=O)C=CC1=O)C(C)C ; *C(=O)[C@H](C)NC(=O)[C@@H](NC(=O)CCCCCN1C(=O)C=CC1=O)C(C)C ; *C(=O)[C@H](CCCNC(N)=O)NC(=O)[C@@H](NC(=O)CCN1C(=O)C=CC1=O)C(C)C ; *C(=O)[C@H](CCCNC(N)=O)NC(=O)[C@H](CC(N)=O)NC(=O)CCN1C(=O)C=CC1=O ; *C(=O)[C@H](C)NC(=O)[C@H](C)NC(=O)CCN1C(=O)C=CC1=O]

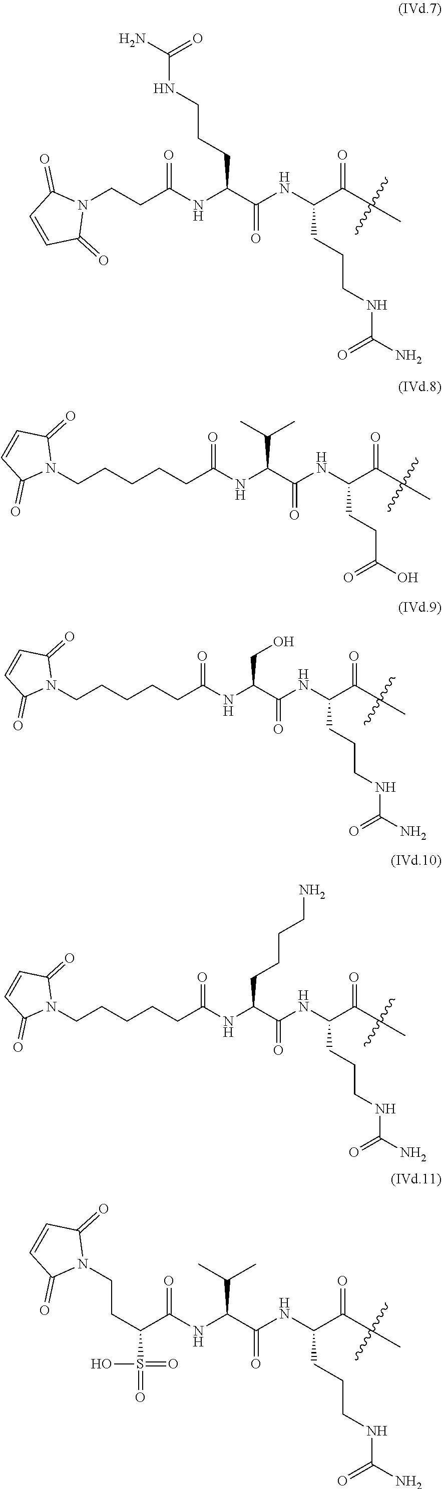 [SMILES: *C(=O)[C@H](CCCNC(N)=O)NC(=O)[C@H](CCCNC(N)=O)NC(=O)CCN1C(=O)C=CC1=O ; *C(=O)[C@H](CCC(=O)O)NC(=O)[C@@H](NC(=O)CCCCCN1C(=O)C=CC1=O)C(C)C ; *C(=O)[C@H](CCCNC(N)=O)NC(=O)[C@H](CO)NC(=O)CCCCCN1C(=O)C=CC1=O ; *C(=O)[C@H](CCCNC(N)=O)NC(=O)[C@H](CCCCN)NC(=O)CCCCCN1C(=O)C=CC1=O ; *C(=O)[C@H](CCCNC(N)=O)NC(=O)[C@@H](NC(=O)[C@@H](CCN1C(=O)C=CC1=O)S(=O)(=O)O)C(C)C]

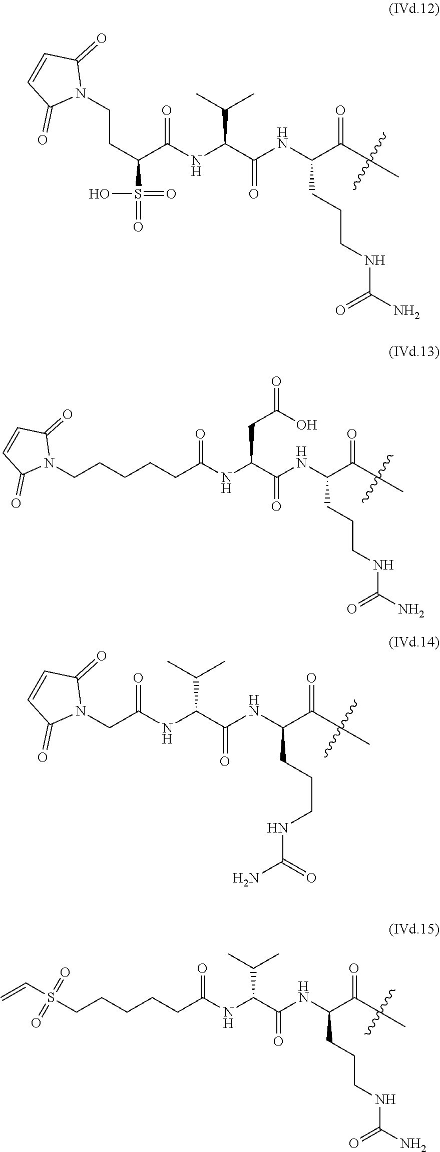 [SMILES: *C(=O)[C@H](CCCNC(N)=O)NC(=O)[C@@H](NC(=O)[C@H](CCN1C(=O)C=CC1=O)S(=O)(=O)O)C(C)C ; *C(=O)[C@H](CCCNC(N)=O)NC(=O)[C@H](CC(=O)O)NC(=O)CCCCCN1C(=O)C=CC1=O ; *C(=O)[C@@H](CCCNC(N)=O)NC(=O)[C@H](NC(=O)CN1C(=O)C=CC1=O)C(C)C ; *C(=O)[C@@H](CCCNC(N)=O)NC(=O)[C@H](NC(=O)CCCCCS(=O)(=O)C=C)C(C)C]

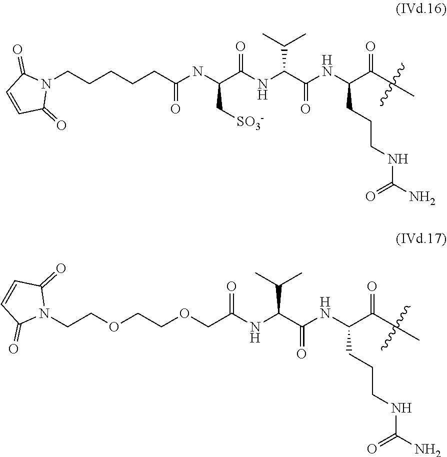

In certain embodiments, the ADC linker comprising structural formula (IVa), (IVb), (IVc), or (IVd) further comprises a carbonate moiety cleavable by exposure to an acidic medium. In particular embodiments, the ADC linker is attached through an oxygen to a cytotoxic and/or cytostatic agent.

### 7.12.2.2. Non-Cleavable Linkers

Although cleavable ADC linkers can provide certain advantages, the ADC linkers comprising the ADCs need not be cleavable. For noncleavable ADC linkers, the release of drug does not depend on the differential properties between the plasma and some cytoplasmic compartments. The release of the drug is postulated to occur after internalization of the ADC via antigen-mediated endocytosis and delivery to lysosomal compartment, where the MBM is degraded to the level of amino acids through intracellular proteolytic degradation. This process releases a drug derivative, which is formed by the drug, the ADC linker, and the amino acid residue to which the ADC linker was covalently attached. The amino acid drug metabolites from conjugates with noncleavable ADC linkers are more hydrophilic and generally less membrane permeable, which leads to less bystander effects and less nonspecific toxicities compared to conjugates with a cleavable ADC linker. In general, ADCs with noncleavable ADC linkers have greater stability in circulation than ADCs with cleavable ADC linkers. Non-cleavable ADC linkers can be alkylene chains, or can be polymeric in nature, such as, for example, based upon polyalkylene glycol polymers, amide polymers, or can include segments of alkylene chains, polyalkylene glycols and/or amide polymers.

A variety of non-cleavable ADC linkers used to link drugs to MBMs have been described. See, Jeffrey et al., 2006, Bioconjug. Chem. 17; 831-840; Jeffrey et al., 2007, Bioorg. Med. Chem. Lett. 17:2278-2280; and Jiang et al., 2005, J. Am. Chem. Soc. 127:11254-11255. All of these ADC linkers can be included in the ADCs of the disclosure.

In certain embodiments, the ADC linker is non-cleavable in vivo, for example an ADC linker according to structural formula (VIa), (VIb), (VIc) or (VId) (as illustrated, the ADC linkers include a group suitable for covalently linking the ADC linker to a MBM:

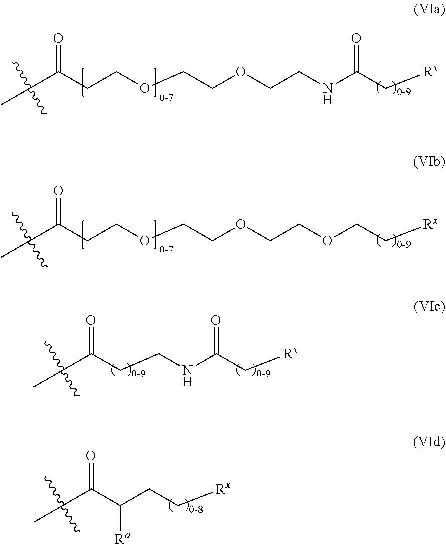

or salts thereof, where: R^{a} is selected from hydrogen, alkyl, sulfonate and methyl sulfonate; R^{x} is a moiety including a functional group capable of covalently linking the ADC linker to a MBM; and [symbol] represents the point of attachment of the ADC linker to a cytotoxic and/or cytostatic agent.

Specific exemplary embodiments of ADC linkers according to structural formula (VIa)-(VId) that can be included in the ADCs include the ADC linkers illustrated below (as illustrated, the ADC linkers include a group suitable for covalently linking the ADC linker to a MBM, and [symbol] represents the point of attachment to a cytotoxic and/or cytostatic agent):

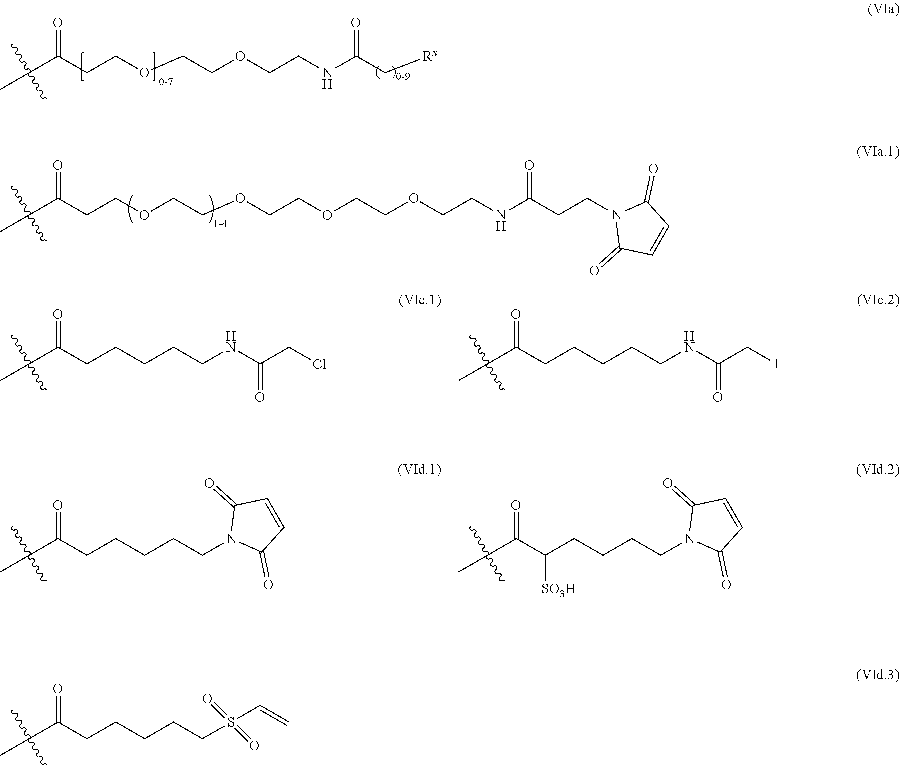

### 7.12.2.3. Groups Used to Attach Linkers to MBMs

A variety of groups can be used to attach ADC linker-drug synthons to MBMs (e.g., BBMs) to yield ADCs. Attachment groups can be electrophilic in nature and include: maleimide groups, activated disulfides, active esters such as NHS esters and HOBt esters, haloformates, acid halides, alkyl and benzyl halides such as haloacetamides. As discussed below, there are also emerging technologies related to “self-stabilizing” maleimides and “bridging disulfides” that can be used in accordance with the disclosure. The specific group used will depend, in part, on the site of attachment to the MBM.

One example of a “self-stabilizing” maleimide group that hydrolyzes spontaneously under MBM conjugation conditions to give an ADC species with improved stability is depicted in the schematic below. See US20130309256 A1; also Lyon et al., Nature Biotech published online, doi:10.1038/nbt.2968.

Normal System:

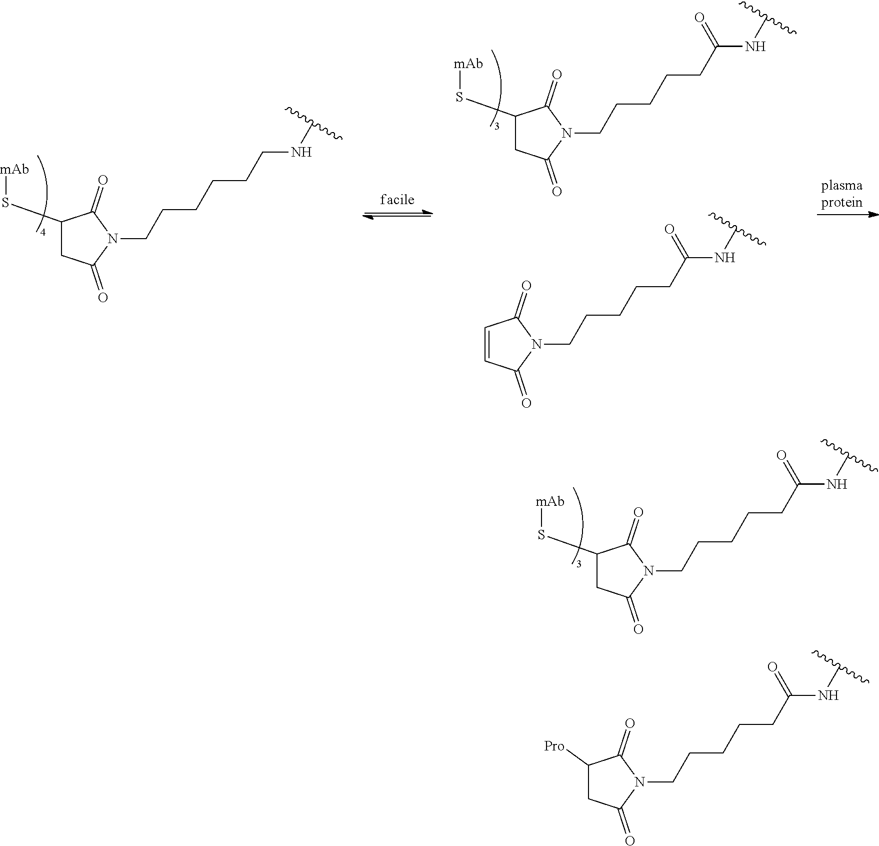

Leads to “DAR loss” over time

SGN MalDPR (Maleimido Dipropylamino) System:

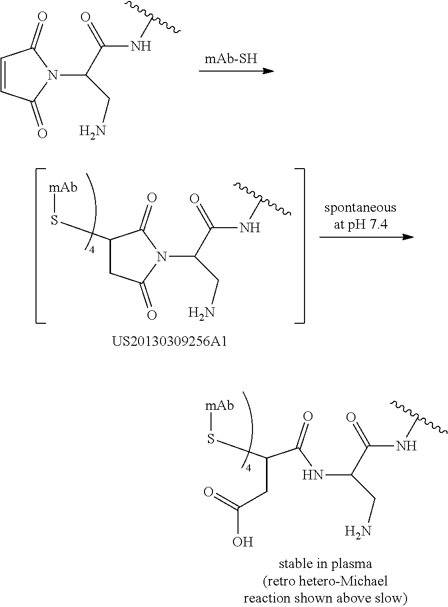

Polytherics has disclosed a method for bridging a pair of sulfhydryl groups derived from reduction of a native hinge disulfide bond. See, Badescu et al., 2014, Bioconjugate Chem. 25:1124-1136. The reaction is depicted in the schematic below. An advantage of this methodology is the ability to synthesize enriched DAR4 ADCs by full reduction of IgGs (to give 4 pairs of sulfhydryls) followed by reaction with 4 equivalents of the alkylating agent. ADCs containing “bridged disulfides” have increased stability.

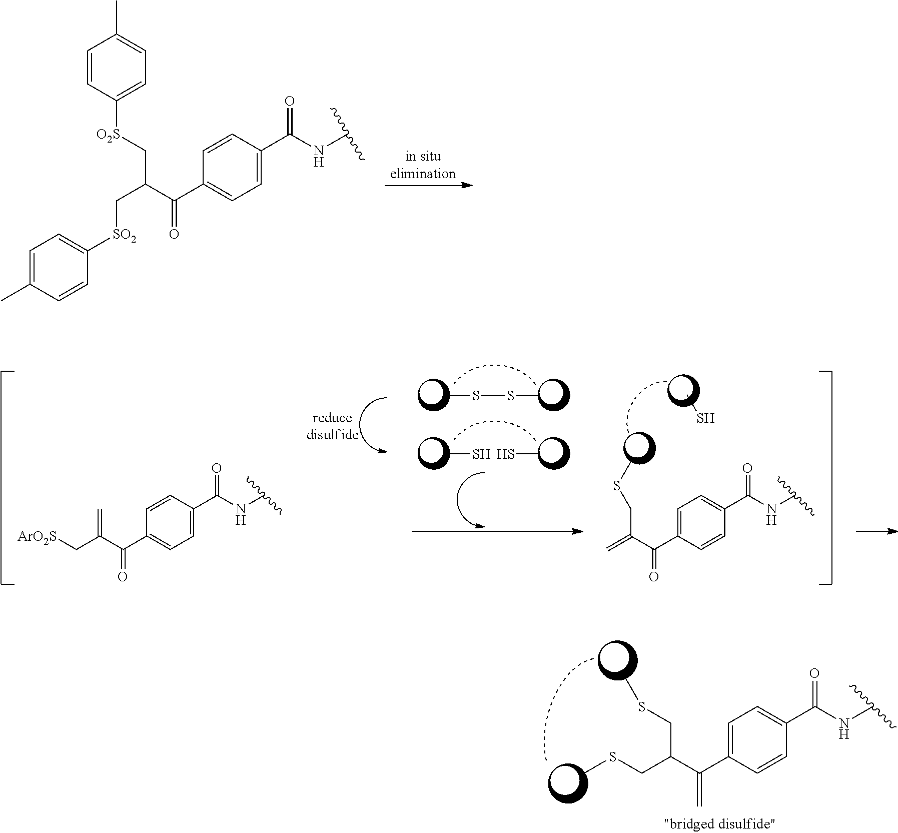

Similarly, as depicted below, a maleimide derivative (1, below) that is capable of bridging a pair of sulfhydryl groups has been developed. See WO2013/085925.

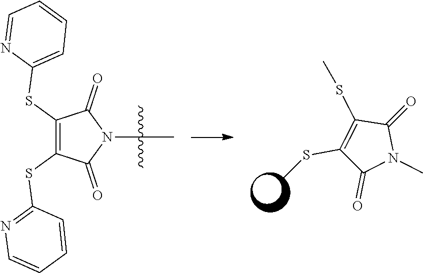

### 7.12.2.4. ADC Linker Selection Considerations

As is known by skilled artisans, the ADC linker selected for a particular ADC can be influenced by a variety of factors, including but not limited to, the site of attachment to the MBM (e.g., lys, cys or other amino acid residues), structural constraints of the drug pharmacophore and the lipophilicity of the drug. The specific ADC linker selected for an ADC should seek to balance these different factors for the specific MBM/drug combination. For a review of the factors that are influenced by choice of ADC linkers in ADCs, see Nolting, Chapter 5 “Linker Technology in Antibody-Drug Conjugates,” In: Antibody-Drug Conjugates: Methods in Molecular Biology, vol. 1045, pp. 71-100, Laurent Ducry (Ed.), Springer Science & Business Medica, L L C, 2013.

For example, ADCs have been observed to effect killing of bystander antigen-negative cells present in the vicinity of the antigen-positive tumor cells. The mechanism of bystander cell killing by ADCs has indicated that metabolic products formed during intracellular processing of the ADCs may play a role. Neutral cytotoxic metabolites generated by metabolism of the ADCs in antigen-positive cells appear to play a role in bystander cell killing while charged metabolites can be prevented from diffusing across the membrane into the medium and therefore cannot affect bystander killing. In certain embodiments, the ADC linker is selected to attenuate the bystander killing effect caused by cellular metabolites of the ADC. In certain embodiments, the ADC linker is selected to increase the bystander killing effect.

The properties of the ADC linker can also impact aggregation of the ADC under conditions of use and/or storage. Typically, ADCs reported in the literature contain no more than 3-4 drug molecules per antibody molecule (see, e.g., Chari, 2008, Acc Chem Res 41:98-107). Attempts to obtain higher drug-to-antibody ratios (“DAR”) often failed, particularly if both the drug and the ADC linker were hydrophobic, due to aggregation of the ADC (King et al., 2002, J Med Chem 45:4336-4343; Hollander et al., 2008, Bioconjugate Chem 19:358-361; Burke et al., 2009 Bioconjugate Chem 20:1242-1250). In many instances, DARs higher than 3-4 could be beneficial as a means of increasing potency. In instances where the cytotoxic and/or cytostatic agent is hydrophobic in nature, it can be desirable to select ADC linkers that are relatively hydrophilic as a means of reducing ADC aggregation, especially in instances where DARS greater than 3-4 are desired. Thus, in certain embodiments, the ADC linker incorporates chemical moieties that reduce aggregation of the ADCs during storage and/or use. An ADC linker can incorporate polar or hydrophilic groups such as charged groups or groups that become charged under physiological pH to reduce the aggregation of the ADCs. For example, an ADC linker can incorporate charged groups such as salts or groups that deprotonate, e.g., carboxylates, or protonate, e.g., amines, at physiological pH.

Exemplary polyvalent ADC linkers that have been reported to yield DARs as high as 20 that can be used to link numerous cytotoxic and/or cytostatic agents to a MBM are described in WO 2009/073445; WO 2010/068795; WO 2010/138719; WO 2011/120053; WO 2011/171020; WO 2013/096901; WO 2014/008375; WO 2014/093379; WO 2014/093394; WO 2014/093640.

In particular embodiments, the aggregation of the ADCs during storage or use is less than about 10% as determined by size-exclusion chromatography (SEC). In particular embodiments, the aggregation of the ADCs during storage or use is less than 10%, such as less than about 5%, less than about 4%, less than about 3%, less than about 2%, less than about 1%, less than about 0.5%, less than about 0.1%, or even lower, as determined by size-exclusion chromatography (SEC).

### 7.12.3. Methods of Making ADCs

The ADCs can be synthesized using chemistries that are well-known. The chemistries selected will depend upon, among other things, the identity of the cytotoxic and/or cytostatic agent(s), the ADC linker and the groups used to attach ADC linker to the MBM. Generally, ADCs according to formula (I) can be prepared according to the following scheme:

D-L-R^{x}+Ab-R^{y}→[D-L-XY]ₙ-Ab (I)

where D, L, Ab, XY and n are as previously defined, and R^{x} and R^{y} represent complementary groups capable of forming a covalent linkages with one another, as discussed above.

The identities of groups R^{x} and R^{y} will depend upon the chemistry used to link synthon D-L-R^{x} to the MBM. Generally, the chemistry used should not alter the integrity of the MBM, for example its ability to bind its target. In some cases, the binding properties of the conjugated antibody will closely resemble those of the unconjugated MBM. A variety of chemistries and techniques for conjugating molecules to biological molecules and in particular to immunoglobulins, whose components are typically building blocks of the MBMs of the disclosure, are well-known. See, e.g., Amon et al., “Monoclonal Antibodies For Immunotargeting Of Drugs In Cancer Therapy,” in: Monoclonal Antibodies And Cancer Therapy, Reisfeld et al. Eds., Alan R. Liss, Inc., 1985; Hellstrom et al., “Antibodies For Drug Delivery,” in: Controlled Drug Delivery, Robinson et al. Eds., Marcel Dekker, Inc., 2nd Ed. 1987; Thorpe, “Antibody Carriers Of Cytotoxic Agents In Cancer Therapy: A Review,” in: Monoclonal Antibodies '84: Biological And Clinical Applications, Pinchera et al., Eds., 1985; “Analysis, Results, and Future Prospective of the Therapeutic Use of Radiolabeled Antibody In Cancer Therapy,” in: Monoclonal Antibodies For Cancer Detection And Therapy, Baldwin et al., Eds., Academic Press, 1985; Thorpe et al., 1982, Immunol. Rev. 62:119-58; PCT publication WO 89/12624. Any of these chemistries can be used to link the synthons to a MBM.

A number of functional groups R^{x} and chemistries useful for linking synthons to accessible lysine residues are known, and include by way of example and not limitation NHS-esters and isothiocyanates.

A number of functional groups R^{x} and chemistries useful for linking synthons to accessible free sulfhydryl groups of cysteine residues are known, and include by way of example and not limitation haloacetyls and maleimides.

However, conjugation chemistries are not limited to available side chain groups. Side chains such as amines can be converted to other useful groups, such as hydroxyls, by linking an appropriate small molecule to the amine. This strategy can be used to increase the number of available linking sites on the antibody by conjugating multifunctional small molecules to side chains of accessible amino acid residues of the MBM. Functional groups R^{x} suitable for covalently linking the synthons to these “converted” functional groups are then included in the synthons.

The MBM can also be engineered to include amino acid residues for conjugation. An approach for engineering MBMs to include non-genetically encoded amino acid residues useful for conjugating drugs in the context of ADCs is described by Axup et al., 2012, Proc Natl Acad Sci USA. 109(40):16101-16106, as are chemistries and functional group useful for linking synthons to the non-encoded amino acids.

Typically, the synthons are linked to the side chains of amino acid residues of the MBM, including, for example, the primary amino group of accessible lysine residues or the sulfhydryl group of accessible cysteine residues. Free sulfhydryl groups can be obtained by reducing interchain disulfide bonds.

For linkages where R^{y} is a sulfhydryl group (for example, when R^{x} is a maleimide), the MBM is generally first fully or partially reduced to disrupt interchain disulfide bridges between cysteine residues.

Cysteine residues that do not participate in disulfide bridges can be engineered into a MBM by modification of one or more codons. Reducing these unpaired cysteines yields a sulfhydryl group suitable for conjugation. In some embodiments, MBMs are engineered to introduce one or more cysteine residues as sites for conjugation to a drug moiety (see, Junutula, et al, 2008, Nat Biotechnol, 26:925-932).

Sites for cysteine substitution can be selected in a constant region to provide sTable 8nd homogeneous conjugates. A MBM can have, for example, two or more cysteine substitutions, and these substitutions can be used in combination with other modification and conjugation methods as described herein. Methods for inserting cysteine at specific locations of an antibody are known, see, e.g., Lyons et al., 1990, Protein Eng., 3:703-708, WO 2011/005481, WO2014/124316, WO 2015/138615. In certain embodiments, a MBM comprises a substitution of one or more amino acids with cysteine on a constant region selected from positions 117, 119, 121, 124, 139, 152, 153, 155, 157, 164, 169, 171, 174, 189, 205, 207, 246, 258, 269, 274, 286, 288, 290, 292, 293, 320, 322, 326, 333, 334, 335, 337, 344, 355, 360, 375, 382, 390, 392, 398, 400 and 422 of a heavy chain, where the positions are numbered according to the EU system. In some embodiments, a MBM comprises a substitution of one or more amino acids with cysteine on a constant region selected from positions 107, 108, 109, 114, 129, 142, 143, 145, 152, 154, 156, 159, 161, 165, 168, 169, 170, 182, 183, 197, 199, and 203 of a light chain, where the positions are numbered according to the EU system, and where the light chain is a human kappa light chain. In certain embodiments a MBM comprises a combination of substitution of two or more amino acids with cysteine on a constant region, where the combinations comprise substitutions at positions 375 of a heavy chain, position 152 of a heavy chain, position 360 of a heavy chain, or position 107 of a light chain and where the positions are numbered according to the EU system. In certain embodiments a MBM comprises a substitution of one amino acid with cysteine on a constant region where the substitution is position 375 of a heavy chain, position 152 of a heavy chain, position 360 of a heavy chain, position 107 of a light chain, position 165 of a light chain or position 159 of a light chain and where the positions are numbered according to the EU system, and where the light chain is a kappa chain.

In particular embodiments, a MBM comprises a combination of substitution of two amino acids with cysteine on a constant regions, where the MBM comprises cysteines at positions 152 and 375 of a heavy chain, where the positions are numbered according to the EU system.

In other particular embodiments, a MBM comprises a substitution of one amino acid with cysteine at position 360 of a heavy chain, where the positions are numbered according to the EU system.

In other particular embodiments, a MBM comprises a substitution of one amino acid with cysteine at position 107 of a light chain, where the positions are numbered according to the EU system, and where the light chain is a kappa chain.

Other positions for incorporating engineered cysteines can include, by way of example and not limitation, positions S112C, S113C, A114C, S115C, A176C, 5180C, S252C, V286C, V292C, S357C, A359C, S398C, S428C (Kabat numbering) on the human IgG₁ heavy chain and positions V110C, S114C, S121C, S127C, S168C, V205C (Kabat numbering) on the human Ig kappa light chain (see, e.g., U.S. Pat. Nos. 7,521,541, 7,855,275 and 8,455,622).

MBMs useful in ADCs disclosed herein the MBM can additionally or alternatively be modified to introduce one or more other reactive amino acids (other than cysteine), including Pcl, pyrrolysine, peptide tags (such as S6, A1 and ybbR tags), and non-natural amino acids, in place of at least one amino acid of the native sequence, thus providing a reactive site on the MBM for conjugation to a drug moiety. For example, MBMs can be modified to incorporate Pcl or pyrrolysine (W. Ou et al., 2011, PNAS, 108(26):10437-10442; WO2014124258) or unnatural amino acids (Axup, et al., 2012, PNAS, 109:16101-16106; for review, see C. C. Liu and P. G. Schultz, 2010, Annu Rev Biochem 79:413-444; Kim, et al., 2013, Curr Opin Chem Biol. 17:412-419) as sites for conjugation to a drug. Similarly, peptide tags for enzymatic conjugation methods can be introduced into a MBM (see, Strop et al. 2013, Chem Biol. 20(2):161-7; Rabuka, 2010, Curr Opin Chem Biol. 14(6):790-6; Rabuka, et al., 2012, Nat Protoc. 7(6):1052-67). One other example is the use of 4′-phosphopantetheinyl transferases (PPTase) for the conjugation of Coenzyme A analogs (WO2013184514). Such modified or engineered MBMs can be conjugated with payloads or linker-payload combinations according to methods known.

As will appreciated by skilled artisans, the number of agents (e.g., cytotoxic and/or cytostatic agents) linked to a MBM molecule can vary, such that a collection of ADCs can be heterogeneous in nature, where some MBMs contain one linked agent, some two, some three, etc. (and some none). The degree of heterogeneity will depend upon, among other things, the chemistries used for linking the agents. For example, where the MBMs are reduced to yield sulfhydryl groups for attachment, heterogeneous mixtures of MBMs having zero, 2, 4, 6 or 8 linked agents per molecule are often produced. Furthermore, by limiting the molar ratio of attachment compound, MBMs having zero, 1, 2, 3, 4, 5, 6, 7 or 8 linked agents per molecule are often produced. Thus, it will be understood that depending upon context, stated drug MBM ratios (DTRs) can be averages for a collection of MBMs. For example, “DTR4” can refer to an ADC preparation that has not been subjected to purification to isolate specific DTR peaks and can comprise a heterogeneous mixture of ADC molecules having different numbers of cytostatic and/or cytotoxic agents attached per MBM (e.g., 0, 2, 4, 6, 8 agents per MBM), but has an average drug-to-MBM ratio of 4. Similarly, in some embodiments, “DTR2” refers to a heterogeneous ADC preparation in which the average drug-to-MBM ratio is 2.

When enriched preparations are desired, MBMs having defined numbers of linked agents (e.g., cytotoxic and/or cytostatic agents) can be obtained via purification of heterogeneous mixtures, for example, via column chromatography, e.g., hydrophobic interaction chromatography.

Purity can be assessed by a variety of known methods. As an example, an ADC preparation can be analyzed via HPLC or other chromatography and the purity assessed by analyzing areas under the curves of the resultant peaks.

### 7.13. Pharmaceutical Compositions

The first and second MBMs (e.g., BBMs) (as well as their conjugates; references to MBMs in this disclosure also refers to conjugates comprising the MBMs, such as ADCs, unless the context dictates otherwise) can be formulated as pharmaceutical compositions comprising a MBM (or a combinations of a first MBM and second MBM), for example containing one or more pharmaceutically acceptable excipients or carriers. To prepare pharmaceutical or sterile compositions comprising a MBM, a MBM preparation can be combined with one or more pharmaceutically acceptable excipient or carrier.

For example, formulations of MBMs can be prepared by mixing MBMs with physiologically acceptable carriers, excipients, or stabilizers in the form of, e.g., lyophilized powders, slurries, aqueous solutions, lotions, or suspensions (see, e.g., Hardman et al., 2001, Goodman and Gilman's The Pharmacological Basis of Therapeutics, McGraw-Hill, New York, N.Y.; Gennaro, 2000, Remington: The Science and Practice of Pharmacy, Lippincott, Williams, and Wilkins, New York, N.Y.; Avis, et al. (eds.), 1993, Pharmaceutical Dosage Forms: General Medications, Marcel Dekker, NY; Lieberman, et al. (eds.), 1990, Pharmaceutical Dosage Forms: Tablets, Marcel Dekker, NY; Lieberman, et al. (eds.), 1990, Pharmaceutical Dosage Forms: Disperse Systems, Marcel Dekker, NY; Weiner and Kotkoskie, 2000, Excipient Toxicity and Safety, Marcel Dekker, Inc., New York, N.Y.).

Selecting an administration regimen for a MBM (or a combination of a first MBM and a second MBM) depends on several factors, including the serum or tissue turnover rate of the MBM(s), the level of symptoms, the immunogenicity of the MBM(s), and the accessibility of the target cells. In certain embodiments, an administration regimen maximizes the amount of MBM(s) delivered to the subject consistent with an acceptable level of side effects. Accordingly, the amount of MBM(s) delivered depends in part on the particular MBM(s) and the severity of the condition being treated. Guidance in selecting appropriate doses of antibodies and small molecules are available (see, e.g., Wawrzynczak, 1996, Antibody Therapy, Bios Scientific Pub. Ltd, Oxfordshire, UK; Kresina (ed.), 1991, Monoclonal Antibodies, Cytokines and Arthritis, Marcel Dekker, New York, N.Y.; Bach (ed.), 1993, Monoclonal Antibodies and Peptide Therapy in Autoimmune Diseases, Marcel Dekker, New York, N.Y.; Baert et al., 2003, New Engl. J. Med. 348:601-608; Milgrom et al., 1999, New Engl. J. Med. 341:1966-1973; Slamon et al., 2001, New Engl. J. Med. 344:783-792; Beniaminovitz et al., 2000, New Engl. J. Med. 342:613-619; Ghosh et al., 2003, New Engl. J. Med. 348:24-32; Lipsky et al., 2000, New Engl. J. Med. 343:1594-1602).

Determination of the appropriate dose is made by the clinician, e.g., using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced.

Actual dosage levels of the MBM(s) in the pharmaceutical compositions of the present disclosure can be varied so as to obtain an amount of the MBM(s) which is effective to achieve the desired therapeutic response for a particular subject, composition, and mode of administration, without being toxic to the subject. The selected dosage level will depend upon a variety of pharmacokinetic factors including the activity of the particular MBM(s), the route of administration, the time of administration, the rate of excretion of the particular MBM(s) being employed, the duration of the treatment, other agents (e.g., active agents such as therapeutic drugs or compounds and/or inert materials used as carriers) in combination with the particular MBM employed, the age, sex, weight, condition, general health and prior medical history of the subject being treated, and like factors known in the medical arts.

Compositions comprising a MBM(s) can be provided by continuous infusion, or by doses at intervals of, e.g., one day, one week, or 1-7 times per week. Doses can be provided intravenously, subcutaneously, topically, orally, nasally, rectally, intramuscular, intracerebrally, or by inhalation. An exemplary dose protocol is one involving the maximal dose or dose frequency that avoids significant undesirable side effects.

An effective amount for a particular subject can vary depending on factors such as the condition being treated, the overall health of the subject, the method route and dose of administration and the severity of side effects (see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK).

The route of administration can be by, e.g., topical or cutaneous application, injection or infusion by intravenous, intraperitoneal, intracerebral, intramuscular, intraocular, intraarterial, intracerebrospinal, intralesional, or by sustained release systems or an implant (see, e.g., Sidman et al., 1983, Biopolymers 22:547-556; Langer et al., 1981, J. Biomed. Mater. Res. 15:167-277; Langer, 1982, Chem. Tech. 12:98-105; Epstein et al., 1985, Proc. Natl. Acad. Sci. USA 82:3688-3692; Hwang et al., 1980, Proc. Natl. Acad. Sci. USA 77:4030-4034; U.S. Pat. Nos. 6,350,466 and 6,316,024). Where necessary, the composition can also include a solubilizing agent and a local anesthetic such as lidocaine to ease pain at the site of the injection. In addition, pulmonary administration can also be employed, e.g., by use of an inhaler or nebulizer, and formulation with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903.

A composition of the present disclosure can also be administered via one or more routes of administration using one or more of a variety of known methods. As will be appreciated by a skilled artisan, the route and/or mode of administration will vary depending upon the desired results. Selected routes of administration for MBMs include intravenous, intramuscular, intradermal, intraperitoneal, subcutaneous, spinal or other general routes of administration, for example by injection or infusion. General administration can represent modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, a composition of the disclosure can be administered via a non-general route, such as a topical, epidermal or mucosal route of administration, for example, intranasally, orally, vaginally, rectally, sublingually or topically. In one embodiment, a MBM(s) is administered by infusion. In another embodiment, a MBM(s) is administered subcutaneously.

If a MBM(s) is administered in a controlled release or sustained release system, a pump can be used to achieve controlled or sustained release (see Langer, supra; Sefton, 1987, CRC Crit. Ref Biomed. Eng. 14:20; Buchwald et al., 1980, Surgery 88:507; Saudek et al., 1989, N. Engl. J. Med. 321:574). Polymeric materials can be used to achieve controlled or sustained release of the therapies of the disclosure (see, e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J., Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105); U.S. Pat. Nos. 5,679,377; 5,916,597; 5,912,015; 5,989,463; 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable. A controlled or sustained release system can be placed in proximity of the prophylactic or therapeutic target, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249:1527-1533). Any technique known to one of skill in the art can be used to produce sustained release formulations comprising one or more MBMs of the disclosure. See, e.g., U.S. Pat. No. 4,526,938, PCT publication WO 91/05548, PCT publication WO 96/20698, Ning et al., 1996, Radiotherapy & Oncology 39:179-189, Song et al., 1995, PDA Journal of Pharmaceutical Science & Technology 50:372-397, Cleek et al., 1997, Pro. Intl Symp. Control. Rel. Bioact. Mater. 24:853-854, and Lam et al., 1997, Proc. Intl Symp. Control Rel. Bioact. Mater. 24:759-760.

If a MBM(s) is administered topically, it can be formulated in the form of an ointment, cream, transdermal patch, lotion, gel, shampoo, spray, aerosol, solution, emulsion, or other form well-known to one of skill in the art. See, e.g., Remington's Pharmaceutical Sciences and Introduction to Pharmaceutical Dosage Forms, 19th ed., Mack Pub. Co., Easton, Pa. (1995). For non-sprayable topical dosage forms, viscous to semi-solid or solid forms comprising a carrier or one or more excipients compatible with topical application and having a dynamic viscosity, in some instances, greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations where the active ingredient, in some instances, in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms if desired. Examples of such additional ingredients are well-known.

If a compositions comprising a MBM(s) is administered intranasally, the MBM(s) can be formulated in an aerosol form, spray, mist or in the form of drops. In particular, prophylactic or therapeutic agents for use according to the present disclosure can be conveniently delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In the case of a pressurized aerosol the dosage unit can be determined by providing a valve to deliver a metered amount. Capsules and cartridges (composed of, e.g., gelatin) for use in an inhaler or insufflator can be formulated containing a powder mix of the compound and a suitable powder base such as lactose or starch.

The MBMs (e.g., BBMs) can be administered in combination therapy regimens, as described in Section 7.15, infra.

In certain embodiments, the MBM(s) can be formulated to ensure proper distribution in vivo. For example, the blood-brain barrier (BBB) excludes many highly hydrophilic compounds. To ensure that the therapeutic compounds of the disclosure cross the BBB (if desired), they can be formulated, for example, in liposomes. For methods of manufacturing liposomes, see, e.g., U.S. Pat. Nos. 4,522,811; 5,374,548; and 5,399,331. The liposomes can comprise one or more moieties which are selectively transported into specific cells or organs, thus enhance targeted drug delivery (see, e.g., Ranade, 1989, J. Clin. Pharmacol. 29:685). Exemplary targeting moieties include folate or biotin (see, e.g., U.S. Pat. No. 5,416,016 to Low et al.); mannosides (Umezawa et al., 1988, Biochem. Biophys. Res. Commun. 153:1038); antibodies (Bloeman et al., 1995, FEBS Lett. 357:140; Owais et al., 1995, Antimicrob. Agents Chemother. 39:180); surfactant protein A receptor (Briscoe et al., 1995, Am. J. Physiol. 1233:134); p 120 (Schreier et al., 1994, J. Biol. Chem. 269:9090); see also Keinanen and Laukkanen, 1994, FEBS Lett. 346:123; Killion and Fidler, 1994, Immunomethods 4:273.

First and second MBMs can be administered simultaneously, in the same or in separate compositions, or sequentially. For sequential administration, the first MBM can be administered first, or the second MBM can be administered first. In some embodiments, the delivery of one MBM is still occurring when the delivery of the other begins, so that there is overlap in terms of administration. This is sometimes referred to herein as “simultaneous” or “concurrent delivery”. For example, the first and second MBM can be administered to a subject at the same time or sequentially in any order at different points in time; however, if not administered at the same time, they should be administered sufficiently close in time so as to provide the desired therapeutic effect.

In some embodiments of either case, the treatment is more effective because of combined administration. For example, the second MBM is more effective, e.g., an equivalent effect is seen with less of the second MBM, or the second MBM reduces symptoms to a greater extent, than would be seen if the second MBM were administered in the absence of the first MBM, or the analogous situation is seen with the first MBM. In some embodiments, delivery is such that the reduction in a symptom, or other parameter related to the disorder is greater than what would be observed with one MBM delivered in the absence of the other. The effect of the two MBMs can be partially additive, wholly additive, or greater than additive. The delivery can be such that an effect of the MBM delivered first is still detectable when the MBM delivered second is delivered.

The combination of a first and second MBM can be administered during periods of active disorder, or during a period of remission or less active disease. When administered in combination, the first and/or second MBM can be administered in an amount or dose that is higher, lower or the same than the amount or dosage of each agent used individually, e.g., as a monotherapy

When used in combination therapy, e.g., as described in Section 7.15, infra, a MBM (or combination of a first MBM and second MBM) and one or more additional agents can be administered to a subject in the same pharmaceutical composition. Alternatively, the MBM (or combination of a first MBM and second MBM) and the additional agent(s) of the combination therapies can be administered concurrently to a subject in separate pharmaceutical compositions.

The therapeutic methods described herein can further comprise carrying out a “companion diagnostic” test whereby a sample from a subject who is a candidate for therapy with a MBM is tested for the expression of a TAA. The companion diagnostic test can be performed prior to initiating therapy with a MBM and/or during a therapeutic regimen with a MBM to monitor the subject's continued suitability for MBM therapy. The agent used in the companion diagnostic can be the MBM itself or another diagnostic agent, for example a labeled monospecific antibody against the TAA or a nucleic acid probe to detect TAA RNA. The sample that can be tested in a companion diagnostic assay can be any sample in which the cells targeted by the MBM can be present, from example a tumor (e.g., a solid tumor) biopsy, lymph, stool, urine, blood or any other bodily fluid that might contain circulating tumor cells.

### 7.14. Therapeutic Indications

### 7.14.1. Cancer

The first and second MBMs (e.g., BBMs) of the disclosure can be used in combination in the treatment of any proliferative disease (e.g., cancer) that expresses a TAA described in Section 7.7 or combination of TAAs described in Section 7.7 (e.g., a cancer characterized by cancerous cells expressing two TAAs on the same cancerous cell or a cancer characterized by cancerous cells expressing a first TAA and a second TAA on different cancerous cells).

In some embodiments, the proliferative disease is a hematologic proliferative disease, for example, a lymphoma, a leukemia, multiple myeloma, a chronic myeloproliferative neoplasm, a macroglobulinemia, a myelodysplastic syndrome, a myelodysplastic/myeloproliferative neoplasm, or a plasmacytic dendritic cell neoplasm.

In some embodiments, the proliferative disease is a lymphoma. In some embodiments, the lymphoma is Hodgkin's lymphoma, for example nodular sclerosing Hodgkin's lymphoma, mixed-cellularity subtype Hodgkin's lymphoma, lymphocyte-rich or lymphocytic predominance Hodgkin's lymphoma, or lymphocyte depleted Hodgkin's lymphoma. In some embodiments, the Hodgkin's lymphoma is nodular sclerosing Hodgkin's lymphoma. In some embodiments, the Hodgkin's lymphoma is mixed-cellularity subtype Hodgkin's lymphoma. In some embodiments, the Hodgkin's lymphoma is lymphocyte-rich or lymphocytic predominance Hodgkin's lymphoma. In some embodiments, the Hodgkin's lymphoma is lymphocyte depleted Hodgkin's lymphoma.

In some embodiments, proliferative disease is non-Hodgkin's lymphoma. In some embodiments, the non-Hodgkin's lymphoma is a B cell lymphoma or a T cell lymphoma. In some embodiments, the non-Hodgkin's lymphoma is a B cell lymphoma. In some embodiments, the non-Hodgkin's lymphoma is a T cell lymphoma. In some embodiments, the non-Hodgkin's lymphoma is diffuse large B-cell lymphoma (DLBCL), follicular lymphoma, chronic lymphocytic leukemia (CLL)/small lymphocytic lymphoma (SLL), mantle cell lymphoma (MCL), marginal zone lymphoma, Burkitt lymphoma, lymphoplasmacytic lymphoma (Waldenstrom macroglobulinemia), primary central nervous system (CNS) lymphoma, primary mediastinal large B-cell lymphoma, mediastinal grey-zone lymphoma (MGZL), splenic marginal zone B-cell lymphoma, extranodal marginal zone B-cell lymphoma of MALT, nodal marginal zone B-cell lymphoma, primary effusion lymphoma, anaplastic large cell lymphoma (ALCL), adult T-cell lymphoma, angiocentric lymphoma, angioimmunoblastic T-cell lymphoma, cutaneous T-cell lymphoma, extranodal natural killer/T-cell lymphoma, enteropathy type intestinal T-cell lymphoma, precursor T-lymphoblastic lymphoma, or unspecified peripheral T-cell lymphoma. In some embodiments, the non-Hodgkin's lymphoma is diffuse large B-cell lymphoma (DLBCL). In some embodiments, the non-Hodgkin's lymphoma is follicular lymphoma. In some embodiments, the non-Hodgkin's lymphoma is chronic lymphocytic leukemia (CLL)/small lymphocytic lymphoma (SLL). In some embodiments, the non-Hodgkin's lymphoma is mantle cell lymphoma (MCL), marginal zone lymphoma. In some embodiments, the non-Hodgkin's lymphoma is Burkitt lymphoma. In some embodiments, the non-Hodgkin's lymphoma is lymphoplasmacytic lymphoma (Waldenstrom macroglobulinemia). In some embodiments, the non-Hodgkin's lymphoma is primary central nervous system (CNS) lymphoma. In some embodiments, the non-Hodgkin's lymphoma is primary mediastinal large B-cell lymphoma. In some embodiments, the non-Hodgkin's lymphoma is mediastinal grey-zone lymphoma (MGZL). In some embodiments, the non-Hodgkin's lymphoma is splenic marginal zone B-cell lymphoma. In some embodiments, the non-Hodgkin's lymphoma is extranodal marginal zone B-cell lymphoma of MALT. In some embodiments, the non-Hodgkin's lymphoma is nodal marginal zone B-cell lymphoma. In some embodiments, the non-Hodgkin's lymphoma is primary effusion lymphoma, anaplastic large cell lymphoma (ALCL). In some embodiments, the non-Hodgkin's lymphoma is adult T-cell lymphoma. In some embodiments, the non-Hodgkin's lymphoma is angiocentric lymphoma. In some embodiments, the non-Hodgkin's lymphoma is angioimmunoblastic T-cell lymphoma. In some embodiments, the non-Hodgkin's lymphoma is cutaneous T-cell lymphoma. In some embodiments, the non-Hodgkin's lymphoma is extranodal natural killer/T-cell lymphoma. In some embodiments, the non-Hodgkin's lymphoma is enteropathy type intestinal T-cell lymphoma. In some embodiments, the non-Hodgkin's lymphoma is precursor T-lymphoblastic lymphoma. In some embodiments, the non-Hodgkin's lymphoma is unspecified peripheral T-cell lymphoma.

In some embodiments, the proliferative disease is a leukemia, for example B-cell acute lymphoid leukemia (BALL), T-cell acute lymphoid leukemia (TALL), acute lymphoid leukemia (ALL), acute myeloid leukemia (AML), chronic myelogenous leukemia (CML), chronic lymphocytic leukemia (CLL), B-cell chronic lymphocytic leukemia (B-CLL), B-cell prolymphocytic leukemia (B-PLL), hairy cell leukemia, precursor B-lymphoblastic leukemia (PB-LBL), large granular lymphocyte leukemia, precursor T-lymphoblastic leukemia (T-LBL), or T-cell chronic lymphocytic leukemia/prolymphocytic leukemia (T-CLL/PLL). In some embodiments, the leukemia is B-cell acute lymphoid leukemia (BALL). In some embodiments, the leukemia is T-cell acute lymphoid leukemia (TALL). In some embodiments, the leukemia is acute lymphoid leukemia (ALL). In some embodiments, the leukemia is acute myeloid leukemia (AML). In some embodiments, the leukemia is chronic myelogenous leukemia (CML). In some embodiments, the leukemia is chronic lymphocytic leukemia (CLL). In some embodiments, the leukemia is B-cell chronic lymphocytic leukemia (B-CLL). In some embodiments, the leukemia is B-cell prolymphocytic leukemia (B-PLL). In some embodiments, the leukemia is hairy cell leukemia. In some embodiments, the leukemia is precursor B-lymphoblastic leukemia (PB-LBL). In some embodiments, the leukemia is large granular lymphocyte leukemia. In some embodiments, the leukemia is precursor T-lymphoblastic leukemia (T-LBL). In some embodiments, the leukemia is T-cell chronic lymphocytic leukemia/prolymphocytic leukemia (T-CLL/PLL).

In some embodiments, the proliferative disease is multiple myeloma.

In some embodiments, the proliferative disease is a chronic myeloproliferative neoplasm.

In some embodiments, the proliferative disease is a macroglobulinemia.

In some embodiments, the proliferative disease is a myelodysplastic syndrome.

In some embodiments, the proliferative disease is a myelodysplastic/myeloproliferative neoplasm.

In some embodiments, the proliferative disease is a plasmacytic dendritic cell neoplasm

In some embodiments, the proliferative disease is adrenocortical carcinoma, anal cancer, appendix cancer, bile duct cancer, bladder cancer, bone cancer, brain cancer, breast cancer, bronchial tumor, carcinoma of unknown primary origin, cervical cancer, a chordoma, colon cancer, colorectal cancer, embryonal tumor, endometrial cancer, ependymoma, esophageal cancer, esthesioneuroblastoma, Ewing sarcoma, eye cancer, malignant fibrous histiocytoma, germ cell tumor, gallbladder cancer, gastric cancer, gastrointestinal carcinoid tumor, gastrointestinal stromal tumor, gestational trophoblastic disease, glioma, head and neck cancer, heart cancer, HER2. In some embodiments, the proliferative disease is adrenocortical carcinoma. In some embodiments, the proliferative disease is anal cancer. In some embodiments, the proliferative disease is appendix cancer. In some embodiments, the proliferative disease is bile duct cancer. In some embodiments, the proliferative disease is bladder cancer. In some embodiments, the proliferative disease is bone cancer. In some embodiments, the proliferative disease is brain cancer. In some embodiments, the proliferative disease is breast cancer. In some embodiments, the proliferative disease is bronchial tumor. In some embodiments, the proliferative disease is carcinoma of unknown primary origin. In some embodiments, the proliferative disease is cervical cancer. In some embodiments, the proliferative disease is a chordoma. In some embodiments, the proliferative disease is colon cancer. In some embodiments, the proliferative disease is colorectal cancer. In some embodiments, the proliferative disease is embryonal tumor. In some embodiments, the proliferative disease is endometrial cancer. In some embodiments, the proliferative disease is ependymoma. In some embodiments, the proliferative disease is esophageal cancer. In some embodiments, the proliferative disease is esthesioneuroblastoma. In some embodiments, the proliferative disease is Ewing sarcoma. In some embodiments, the proliferative disease is eye cancer. In some embodiments, the proliferative disease is malignant fibrous histiocytoma. In some embodiments, the proliferative disease is germ cell tumor. In some embodiments, the proliferative disease is gallbladder cancer. In some embodiments, the proliferative disease is gastric cancer. In some embodiments, the proliferative disease is gastrointestinal carcinoid tumor. In some embodiments, the proliferative disease is gastrointestinal stromal tumor. In some embodiments, the proliferative disease is gestational trophoblastic disease. In some embodiments, the proliferative disease is glioma. In some embodiments, the proliferative disease is head and neck cancer. In some embodiments, the proliferative disease is heart cancer. In some embodiments, the proliferative disease is HER In some embodiments, the proliferative disease is hypopharyngeal cancer. In some embodiments, the proliferative disease is Kaposi sarcoma. In some embodiments, the proliferative disease is kidney cancer. In some embodiments, the proliferative disease is Langerhans cell histiocytosis. In some embodiments, the proliferative disease is laryngeal cancer. In some embodiments, the proliferative disease is lip and oral cavity cancer. In some embodiments, the proliferative disease is liver cancer. In some embodiments, the proliferative disease is lung cancer. In some embodiments, the proliferative disease is mesothelioma. In some embodiments, the proliferative disease is metastatic squamous neck cancer with occult primary. In some embodiments, the proliferative disease is midline tract carcinoma involving NUT gene. In some embodiments, the proliferative disease is mouth cancer. In some embodiments, the proliferative disease is nasal cavity cancer. In some embodiments, the proliferative disease is nasopharyngeal cancer. In some embodiments, the proliferative disease is neuroblastoma. In some embodiments, the proliferative disease is oropharyngeal cancer. In some embodiments, the proliferative disease is ovarian cancer. In some embodiments, the proliferative disease is pancreatic cancer. In some embodiments, the proliferative disease is para-nasal sinus cancer. In some embodiments, the proliferative disease is paraganglioma. In some embodiments, the proliferative disease is parathyroid cancer. In some embodiments, the proliferative disease is penile cancer. In some embodiments, the proliferative disease is pharyngeal cancer. In some embodiments, the proliferative disease is pituitary cancer. In some embodiments, the proliferative disease is pleuropulmonary blastoma. In some embodiments, the proliferative disease is prostate cancer. In some embodiments, the proliferative disease is rectal cancer. In some embodiments, the proliferative disease is renal cell cancer. In some embodiments, the proliferative disease is renal pelvis and ureter cancer. In some embodiments, the proliferative disease is retinoblastoma. In some embodiments, the proliferative disease is a rhabdoid tumor. In some embodiments, the proliferative disease is salivary gland cancer. In some embodiments, the proliferative disease is skin cancer. In some embodiments, the proliferative disease is small intestine cancer. In some embodiments, the proliferative disease is soft tissue sarcoma. In some embodiments, the proliferative disease is spinal cord tumor. In some embodiments, the proliferative disease is stomach cancer. In some embodiments, the proliferative disease is teratoid tumor. In some embodiments, the proliferative disease is testicular cancer. In some embodiments, the proliferative disease is throat cancer. In some embodiments, the proliferative disease is thymoma. In some embodiments, the proliferative disease is thymic carcinoma. In some embodiments, the proliferative disease is thyroid cancer. In some embodiments, the proliferative disease is urethral cancer. In some embodiments, the proliferative disease is uterine cancer. In some embodiments, the proliferative disease is vaginal cancer. In some embodiments, the proliferative disease is vulvar cancer. In some embodiments, the proliferative disease is Wilms tumor.

### 7.14.2. Autoimmune Disorders

The first and second MBMs (e.g., BBMs) of the disclosure can be used in combination in the treatment of autoimmune disorders, which can result from the loss of B-cell tolerance and the inappropriate production of autoantibodies. Autoimmune disorders that can be treated with combinations of first and second MBMs of the disclosure include systemic lupus erythematosus (SLE), Sjögren's syndrome, scleroderma, rheumatoid arthritis (RA), juvenile idiopathic arthritis, graft versus host disease, dermatomyositis, type I diabetes mellitus, Hashimoto's thyroiditis, Graves's disease, Addison's disease, celiac disease, Crohn's Disease, pernicious anaemia, pemphigus vulgaris, vitiligo, autoimmune haemolytic anaemia, idiopathic thrombocytopenic purpura, giant cell arteritis, myasthenia gravis, multiple sclerosis (MS) (e.g., relapsing-remitting MS (RRMS)), glomerulonephritis, Goodpasture's syndrome, bullous pemphigoid, colitis ulcerosa, Guillain-Barré syndrome, chronic inflammatory demyelinating polyneuropathy, anti-phospholipid syndrome, narcolepsy, sarcoidosis, and Wegener's granulomatosis.

In some embodiments, combinations of first and second MBMs are used to treat systemic lupus erythematosus (SLE).

In some embodiments, combinations of first and second MBMs are used to treat Sjögren's syndrome.

In some embodiments, combinations of first and second MBMs are used to treat scleroderma.

In some embodiments, combinations of first and second MBMs are used to treat rheumatoid arthritis (RA).

In some embodiments, combinations of first and second MBMs are used to treat juvenile idiopathic arthritis.

In some embodiments, combinations of first and second MBMs are used to treat graft versus host disease.

In some embodiments, combinations of first and second MBMs are used to treat dermatomyositis.

In some embodiments, combinations of first and second MBMs are used to treat type I diabetes mellitus.

In some embodiments, combinations of first and second MBMs are used to treat Hashimoto's thyroiditis.

In some embodiments, combinations of first and second MBMs are used to treat Graves's disease.

In some embodiments, combinations of first and second MBMs are used to treat Addison's disease.

In some embodiments, combinations of first and second MBMs are used to treat celiac disease.

In some embodiments, combinations of first and second MBMs are used to treat Crohn's Disease.

In some embodiments, combinations of first and second MBMs are used to treat pernicious anaemia.

In some embodiments, combinations of first and second MBMs are used to treat pemphigus vulgaris.

In some embodiments, combinations of first and second MBMs are used to treat vitiligo.

In some embodiments, combinations of first and second MBMs are used to treat autoimmune haemolytic anaemia.

In some embodiments, combinations of first and second MBMs are used to treat idiopathic thrombocytopenic purpura.

In some embodiments, combinations of first and second MBMs are used to treat giant cell arteritis.

In some embodiments, combinations of first and second MBMs are used to treat myasthenia gravis.

In some embodiments, combinations of first and second MBMs are used to treat multiple sclerosis (MS). In some embodiments, the MS is relapsing-remitting MS (RRMS).

In some embodiments, combinations of first and second MBMs are used to treat glomerulonephritis.

In some embodiments, combinations of first and second MBMs are used to treat Goodpasture's syndrome.

In some embodiments, combinations of first and second MBMs are used to treat bullous pemphigoid.

In some embodiments, combinations of first and second MBMs are used to treat colitis ulcerosa.

In some embodiments, combinations of first and second MBMs are used to treat Guillain-Barré syndrome.

In some embodiments, combinations of first and second MBMs are used to treat chronic inflammatory demyelinating polyneuropathy.

In some embodiments, combinations of first and second MBMs are used to treat anti-phospholipid syndrome.

In some embodiments, combinations of first and second MBMs are used to treat narcolepsy.

In some embodiments, combinations of first and second MBMs are used to treat sarcoidosis.

In some embodiments, combinations of first and second MBMs are used to treat Wegener's granulomatosis.

### 7.15. Combination Therapy

Combinations of first and second MBMs (e.g., a BBM) of the disclosure can be used in combination with other known agents and therapies. For example, a combination of a first MBM and a second MBM can be used in treatment regimens in combination with surgery, chemotherapy, antibodies, radiation, peptide vaccines, steroids, cytoxins, proteasome inhibitors, immunomodulatory drugs (e.g., IMiDs), BH3 mimetics, cytokine therapies, stem cell transplant or any combination thereof. Without being bound by theory, it is believed that one of the advantages of the combinations of MBMs of the disclosure is that they can circumvent the need for administering other antibodies to a subject suffering from a cancer. Accordingly, in certain embodiments, the one or more additional agents do not include an antibody.

For convenience, an agent that is used in combination with a MBM is referred to herein as an “additional” agent.

Administered “in combination,” as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. For example, each therapy can be administered to a subject at the same time or sequentially in any order at different points in time; however, if not administered at the same time, they should be administered sufficiently close in time so as to provide the desired therapeutic effect.

A first and/or second MBM and one or more additional agents can be administered simultaneously, in the same or in separate compositions, or sequentially. For sequential administration, the first and/or second MBM can be administered first, and the additional agent can be administered after the first and/or second MBM, or the additional agent can be administered before the first and/or second MBM or the additional agent can be administered between the first MBM and second MBM (where the first MBM is administered first or last).

The first and/or second MBM and the additional agent(s) can be administered to a subject in any appropriate form and by any suitable route. In some embodiments, the routes of administration are the same. In other embodiments the routes of administration are different.

In other embodiments, the delivery of one treatment ends before the delivery of the other treatment begins.

In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

The combination of a first and second MBM and/or additional agents can be administered during periods of active disorder, or during a period of remission or less active disease. A combination of first and second MBM scan be administered before the treatment with the additional agent(s), concurrently with the treatment with the additional agent(s), post-treatment with the additional agent(s), or during remission of the disorder.

When administered in combination, a combination of first and second MBMs and/or the additional agent(s) can be administered in an amount or dose that is higher, lower or the same than the amount or dosage of each agent used individually, e.g., as a monotherapy.

The additional agent(s) of the combination therapies of the disclosure can be administered to a subject concurrently. Each therapy can be administered to a subject together or separately, in any appropriate form and by any suitable route.

A combination of first and second MBMs and an additional agent(s) can be cyclically administered. Cycling therapy involves the administration of a first therapy (e.g., a first prophylactic or therapeutic agent) for a period of time, followed by the administration of a second therapy (e.g., a second prophylactic or therapeutic agent) for a period of time, optionally, followed by the administration of a third therapy (e.g., prophylactic or therapeutic agent) for a period of time and so forth, and repeating this sequential administration, i.e., the cycle in order to reduce the development of resistance to one of the therapies, to avoid or reduce the side effects of one of the therapies, and/or to improve the efficacy of the therapies.

In certain instances, the one or more additional agents are other anti-cancer agents, anti-allergic agents, anti-nausea agents (or anti-emetics), pain relievers, cytoprotective agents, and combinations thereof.

In one embodiment, a combination of first and second MBMs is administered in combination with an anti-cancer agent (e.g., a chemotherapeutic agent). Exemplary chemotherapeutic agents include an anthracycline (e.g., doxorubicin (e.g., liposomal doxorubicin)), a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, tositumomab, obinutuzumab, ofatumumab, daratumumab, elotuzumab), an antimetabolite (including, e.g., folic acid antagonists, pyrimidine analogs, purine analogs and adenosine deaminase inhibitors (e.g., fludarabine)), an mTOR inhibitor, a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide).

General chemotherapeutic agents considered for use in combination therapies include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

Anti-cancer agents of particular interest for combinations with the MBMs of the present disclosure include: anthracyclines; alkylating agents; antimetabolites; drugs that inhibit either the calcium dependent phosphatase calcineurin or the p70S6 kinase FK506) or inhibit the p70S6 kinase; mTOR inhibitors; immunomodulators; vinca alkaloids; proteasome inhibitors; GITR agonists (e.g., GWN323); protein tyrosine phosphatase inhibitors; a CDK4 kinase inhibitor; a BTK inhibitor; a MKN kinase inhibitor; a DGK kinase inhibitor; an oncolytic virus; a BH3 mimetic, and cytokine therapies.

A combination of a first and second MBM can be administered in combination with one or more anti-cancer agents that prevent or slow shedding of an antigen targeted by one or more of the ABMs of the first and/or second MBM, thereby reducing the amount of soluble antigen and/or increasing the amount of cell surface bound antigen. For example, MBMs can be administered in combination with an ADAM10/17 inhibitor (e.g., INCB7839), e.g., to block shedding of an antigen released from cancer a cell by ADAM10/17, or in combination with a phospholipase inhibitor, e.g., to block shedding of an antigen released from a cancer cell by a phospholipase.

Exemplary alkylating agents include, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

Exemplary mTOR inhibitors include, e.g., temsirolimus; ridaforolimus (formally known as deferolimus, (1R,2R,4S)-4-[(2R)-2 [(1R,9S,12S,15R,16E,18R,19R,21R, 23S,24E,26E,28Z,30S,32S,35R)-1,18-dihydroxy-19,30-dimethoxy-15,17,21,23, 29,35-hexamethyl-2,3,10,14,20-pentaoxo-11,36-dioxa-4-azatricyclo[30.3.1.04,9] hexatriaconta-16,24,26,28-tetraen-12-yl]propyl]-2-methoxycyclohexyl dimethylphosphinate, also known as AP23573 and MK8669, and described in PCT Publication No. WO 03/064383); everolimus (Afinitor® or RAD001); rapamycin (AY22989, Sirolimus®); simapimod (CAS 164301-51-3); emsirolimus, (5-{2,4-Bis[(3S)-3-methylmorpholin-4-yl]pyrido[2,3-d]pyrimidin-7-yl}-2-methoxyphenyl)methanol (AZD8055); 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502, CAS 1013101-36-4); and N2-[1,4-dioxo-4-[[4-(4-oxo-8-phenyl-4H-1-benzopyran-2-yl)morpholinium-4-yl]methoxy]butyl]-L-arginylglycyl-L-α-aspartylL-serine-(SEQ ID NO:), inner salt (SF1126, CAS 936487-67-1), and XL765.

Exemplary immunomodulators include, e.g., afutuzumab (available from Roche®); pegfilgrastim (Neulasta®); lenalidomide (CC-5013, Revlimid®); IMIDs (such as thalidomide (Thalomid®), lenalidomide, pomalidomide, and apremilast), actimid (CC4047); and IRX-2 (mixture of human cytokines including interleukin 1, interleukin 2, and interferon γ, CAS 951209-71-5, available from IRX Therapeutics).

Exemplary anthracyclines include, e.g., doxorubicin (Adriamycin® and Rubex®); bleomycin (Ienoxane®); daunorubicin (dauorubicin hydrochloride, daunomycin, and rubidomycin hydrochloride, Cerubidine®); daunorubicin liposomal (daunorubicin citrate liposome, DaunoXome®); mitoxantrone (DHAD, Novantrone®); epirubicin (Ellence™); idarubicin (Idamycin®, Idamycin PFS®); mitomycin C (Mutamycin®); geldanamycin; herbimycin; ravidomycin; and desacetylravidomycin.

Exemplary vinca alkaloids include, e.g., vinorelbine tartrate (Navelbine®), Vincristine (Oncovin®), and Vindesine (Eldisine®)); vinblastine (also known as vinblastine sulfate, vincaleukoblastine and VLB, Alkaban-AQ® and Velban®); and vinorelbine (Navelbine®).

Exemplary proteasome inhibitors include bortezomib (Velcade®); carfilzomib (PX-171-007, (S)-4-Methyl-N—((S)-1-(((S)-4-methyl-1-((R)-2-methyloxiran-2-yl)-1-oxopentan-2-yl)amino)-1-oxo-3-phenylpropan-2-yl)-2-((S)-2-(2-morpholinoacetamido)-4-phenylbutanamido)-pentanamide); marizomib (NPI-0052); ixazomib citrate (MLN-9708); delanzomib (CEP-18770); and O-Methyl-N-[(2-methyl-5-thiazolyl)carbonyl]-L-seryl-O-methyl-N—R1S)-2-[(2R)-2-methyl-2-oxiranyl]-2-oxo-1-(phenylmethyl)ethyl]-L-serinamide (ONX-0912).

Exemplary BH3 mimetics include venetoclax, ABT-737 (4-{4-[(4′-Chloro-2-biphenyl)methyl]-1-piperazinyl}-N-[(4-{[(2R)-4-(dimethylamino)-1-(phenylsulfanyl)-2-butanyl]amino}-3-nitrophenyl)sulfonyl]benzamide and navitoclax (formerly ABT-263).

Exemplary cytokine therapies include interleukin 2 (IL-2) and interferon-alpha (IFN-alpha).

In certain aspects, “cocktails” of different chemotherapeutic agents are administered as the additional agent(s).

Second MBMs having an ABM4 that binds to CD3 can be administered in combination with an agent which reduces or ameliorates a side effect associated with the administration of a MBM that binds to CD3. Side effects associated with the administration of CD3 binders include, but are not limited to, cytokine release syndrome (“CRS”) and hemophagocytic lymphohistiocytosis (HLH), also termed Macrophage Activation Syndrome (MAS). Symptoms of CRS can include high fevers, nausea, transient hypotension, hypoxia, and the like. CRS can include clinical constitutional signs and symptoms such as fever, fatigue, anorexia, myalgias, arthalgias, nausea, vomiting, and headache. CRS can include clinical skin signs and symptoms such as rash. CRS can include clinical gastrointestinal signs and symptoms such as nausea, vomiting and diarrhea. CRS can include clinical respiratory signs and symptoms such as tachypnea and hypoxemia. CRS can include clinical cardiovascular signs and symptoms such as tachycardia, widened pulse pressure, hypotension, increased cardiac output (early) and potentially diminished cardiac output (late). CRS can include clinical coagulation signs and symptoms such as elevated d-dimer, hypofibrinogenemia with or without bleeding. CRS can include clinical renal signs and symptoms such as azotemia. CRS can include clinical hepatic signs and symptoms such as transaminitis and hyperbilirubinemia. CRS can include clinical neurologic signs and symptoms such as headache, mental status changes, confusion, delirium, word finding difficulty or frank aphasia, hallucinations, tremor, dymetria, altered gait, and seizures.

Accordingly, the methods described herein can comprise administering a second MBM having an ABM4 that binds to CD3 to a subject and further administering one or more agents to manage elevated levels of a soluble factor resulting from treatment with the MBM. In one embodiment, the soluble factor elevated in the subject is one or more of IFN-γ, TNFα, IL-2 and IL-6. In an embodiment, the factor elevated in the subject is one or more of IL-1, GM-CSF, IL-10, IL-8, IL-5 and fraktalkine. Therefore, an agent administered to treat this side effect can be an agent that neutralizes one or more of these soluble factors. In one embodiment, the agent that neutralizes one or more of these soluble forms is an antibody or antigen binding fragment thereof. Examples of such agents include, but are not limited to a steroid (e.g., corticosteroid), an inhibitor of TNFα, and inhibitor of IL-1R, and an inhibitor of IL-6. An example of a TNFα inhibitor is an anti-TNFα antibody molecule such as, infliximab, adalimumab, certolizumab pegol, and golimumab. Another example of a TNFα inhibitor is a fusion protein such as entanercept. Small molecule inhibitor of TNFα include, but are not limited to, xanthine derivatives (e.g. pentoxifylline) and bupropion. An example of an IL-6 inhibitor is an anti-IL-6 antibody molecule such as tocilizumab (toc), sarilumab, elsilimomab, ONTO 328, ALD518/BMS-945429, ONTO 136, CPSI-2364, CDP6038, VX30, ARGX-109, FE301, and FM101. In one embodiment, the anti-IL-6 antibody molecule is tocilizumab. An example of an IL-1R based inhibitor is anakinra.

In some embodiment, the subject is administered a corticosteroid, such as, e.g., methylprednisolone, hydrocortisone, among others. In some embodiments, the subject is administered a corticosteroid, e.g., methylprednisolone, hydrocortisone, in combination with Benadryl and Tylenol prior to the administration of a second MBM that binds CD3 to mitigate the CRS risk.

In some embodiments, the subject is administered a vasopressor, such as, e.g., norepinephrine, dopamine, phenylephrine, epinephrine, vasopressin, or any combination thereof.

In an embodiment, the subject can be administered an antipyretic agent. In an embodiment, the subject can be administered an analgesic agent.

### 8. EXAMPLES

### 8.1. Example 1: Production and Characterization of Anti-BCMA Antibodies

Anti-BCMA antibodies that are cross-reactive with both human and cynomolgus BCMA were identified using phage display. Affinity maturation of a selected antibody, designated R1F2 in Table 15, was performed to produce antibodies having increased affinity for BCMA. Several additional anti-BCMA antibodies derived from the parental R1F2 antibody were obtained. These antibodies are designated as “AB1/AB2 Family” binders in Table 15. Another antibody separately identified using phage display, designated PI-61 in Table 15, was also subjected to affinity maturation to produce clones having increased affinity for BCMA. Several additional anti-BCMA antibodies derived from the parental PI-61 antibody were obtained. These antibodies are designated as “AB3 Family” binders in Table 15.

Anti-BCMA×anti-CD3 bispecific antibodies having VH and VL sequences of AB1, AB2, and AB3 were produced. The bispecific antibodies were found to be active in in vitro RTCC assays with BCMA+ multiple myeloma cell lines and found to have anti-tumor activity in a KMS11-Luc multiple myeloma orthotopic tumor model in NSG mice.

### 8.2. Example 2: Engineering CD58 for Improved Stability

### 8.2.1. Background

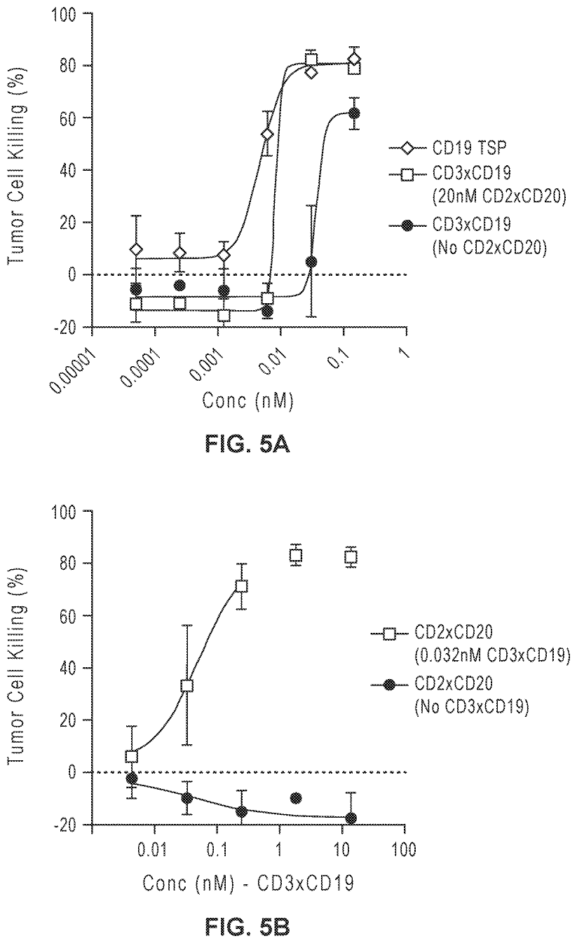

Human CD58 contains a signal peptide of 29 amino acids and two Ig-like domains. The most N-terminal Ig-like domain, referred to as domain 1, is of V-type, similar to a variable region of an antibody, and the second domain, named domain 2, is of C-type, is similar to a constant regions of an antibody. A schematic overview of the CD58 domain structure is shown in FIG. 5.

Domain 1 of CD58, which interacts with CD2, can be used in lieu of an anti-CD2 antibody binding fragment in multispecific binding molecules. However, CD58 exhibits lower stability than immunoglobulins.

In order to improve stability of human CD58 domain 1, the protein was engineered to include a pair of cysteine that form a disulfide bridge upon expression to stabilize the molecule.

Four different pairs of amino acids were engineered to be replaced by cysteines: (1) V45 and M105, (2) V45 and M114, (3) V54 and G88 and (4) W56 and L90.

### 8.2.2. Materials and Methods

### 8.2.2.1. Recombinant Expression

To assess the binding and biophysical characteristics, the CD58 disulfide variants were transiently produced and purified from HEK293 cells along with the CD2 extracellular domain. All plasmids were codon optimized for mammalian expression. Human and cyno CD2 constructs were produced with a C-terminal Avi-Tag and a N terminal 8×his tag (SEQ ID NO: 1322) followed by a EVNLYFQS sequence (SEQ ID NO: 1323) for cleavage of the histag after purification. CD2 constructs were site selectively biotinylated during expression via co-transfection of a plasmid encoding the BirA enzyme. CD58 was expressed with a C-terminal 8×his tag (SEQ ID NO: 1322). Transient expression and purification in HEK293F cells was performed with standard methodology. The sequences are shown in Table 25.

TABLE 25

| Pro- | | SEQ |
|---|---|---|
| tein | | ID |
| Name | AA Sequence | NO: |
| Human | SKEITNALETWGALGQDINLDIPSFQMSDDIDDIKWEKTS | 771 |
| CD2 | DKKKIAQFRKEKETFKEKDTYKLFKNGTLKIKHLKTDDQ | |
| | DIYKVSIYDTKGKNVLEKIFDLKIQERVSKPKISWTCINTT | |
| | LTCEVMNGTDPELNLYQDGKHLKLSQRVITHKWTTSLS | |
| | AKFKCTAGNKVSKESSVEPVSCPEKGLDGGGGSGLNDI | |
| | FEAQKIEWHE | |
| Cyno | SKEIRNALETWGALGQDIDLDIPSFQMSDDIDDIRWEKT | 772 |
| CD2 | SDKKKIAQFRKEKETFEEKDAYKLFKNGTLKIKHLKIHDQ | |
| | DSYKVSIYDTKGKNVLEKTFDLKIQERVSEPKISWTCINT | |
| | TLTCEVMNGTDPELNLYQDGKHVKLSQRVITHKWTTSL | |
| | SAKFKCTAGNKVSKESRMETVSCPEKGLDGGGGSGLN | |
| | DIFEAQKIEWHE | |
| CD58 | SQQIYGVVYGNVTFHVPSNVPLKEVLWKKQKDKVAELE | 773 |
| Full | NSEFRAFSSFKNRVYLDTVSGSLTIYNLTSSDEDEYEME | |
| ECD | SPNITDTMKFFLYVLESLPSPTLTCALTNGSIEVQCMIPE | |
| | HYNSHRGLIMYSWDCPMEQCKRNSTSIYFKMENDLPQ | |
| | KIQCTLSNPLFNTTSSIILTTCIPSSGHSRHRGGGGSHHH | |
| | HHHHH | |
| CD58_ | SQQIYGVVYGNVTFHVPSNVPLKEVLWKKQKDKVAELE | 774 |
| IgV | NSEFRAFSSFKNRVYLDTVSGSLTIYNLTSSDEDEYEME | |
| | SPNITDTMKFFLYVLESGGGGSHHHHHHHH | |
| Igv | SQQIYGVVYGNVTFHCPSNVPLKEVLWKKQKDKVAELE | 775 |
| V45C_ | NSEFRAFSSFKNRVYLDTVSGSLTIYNLTSSDEDEYECE | |
| M105C | SPNITDTMKFFLYVLESGGGGSHHHHHHHH | |
| Igv | SQQIYGVVYGNVTFHVPSNVPLKECLWKKQKDKVAELE | 776 |
| V54C_ | NSEFRAFSSFKNRVYLDTVSCSLTIYNLTSSDEDEYEME | |
| G88C | SPNITDTMKFFLYVLESGGGGSHHHHHHHH | |
| Igv | SQQIYGVVYGNVTFHCPSNVPLKEVLWKKQKDKVAELE | 777 |
| V45C_ | NSEFRAFSSFKNRVYLDTVSGSLTIYNLTSSDEDEYEME | |
| M114C | SPNITDTCKFFLYVLESGGGGSHHHHHHHH | |

For expression, transfection was performed using PEI as transfection reagent. For small scale (<5L) transfections, cells were grown in shake flasks on an orbital shaker (100 rpm) in a humidified incubator (85%) at 8% CO2). Transfection was done with a ratio of 1 DNA:3 PEI. 1 mg/L culture of plasmid was used for transfection at 2.0 million cells/mL in Expi293 medium. After 5 days of expression, the culture was centrifuged and filtrated. Purification was performed via Nickel-NTA batch binding using 1 ml resin/100 mL supernatant. The protein was allowed to bind for a minimum of 2 hours with gentle mixing, and the mixture was loaded onto a gravity filtration column. The resin was washed with 30 CV of PBS. Proteins were eluted with imidazole. The eluted protein was concentrated and finally purified via a preparative size exclusion chromatography (Hi Load 16/60 Superdex 75 grade column, GE Healthcare Life Sciences, Uppsala, Sweden). To confirm that the identity of the proteins expressed matched the predicted masses for the primary amino acid sequences, proteins were analyzed by high-performance liquid chromatography coupled to mass spectrometry.

### 8.2.2.2. Stability

Disulfide stabilized variants were assessed for improved thermal stability using both differential scanning calorimetry (DSC) and differential scanning fluorimetry (DSF) using standard techniques. For DSF, 1-3 ug of each construct was add to 1× Sypro Orange (Thermo-Fisher) in 25 ul total volume in 96-well PCR plate. Using a Bio-Rad CFX96 RT-PCR system equipped with C1000 Thermal Cycler, the temperature was increased from 25° C. to 95° C. at 0.5° C./minute and the fluorescence monitored. The manufacturer-supplied software was used to determine Tm.

For DSC, all samples were dialyzed into HEPES-buffered saline (HBS) and diluted to final concentration of 0.5 mg/mL. Tm and Tonset were determined using a MicroCal VP-Capillary DSC system (Malvern) by increasing temperature from 25° C. to 100° C. at 1° C./minute with a filtering period of 2 seconds and a mid-gain setting.

### 8.2.2.3. Binding Affinity

To ensure the binding affinity remained uncompromised by the additional of the stabilizing disulfide variance, isothermal calorimetry (ITC) was performed on the resulting recombinant CD58 proteins to determine their apparent KD and binding stoichiometry (n) to recombinant human CD2.

Briefly, recombinant human CD2 and recombinant human CD58 variants were dialyzed into HEPES-buffered saline (HBS). CD2 was diluted to final concentration of 100 μM, CD58 variants were diluted to 10 μM. CD2 was titrated into 10 μM of CD58 variants via multiple injections and ΔH (kcal/mole) determined using a MicroCal VP-ITC isothermal titration calorimeter (Malvern). Titrations of CD2 into HBS were used as a reference and KD and n determined from the resulting data.

### 8.2.3. Results

Results for both DSF and DSC measurements for the constructs are shown in Table 26 below.

TABLE 26

| | By Differential | By Differential | |
|---|---|---|---|
| | Scanning | Scanning | |
| | Fluorimetry (DSF) | Calorimetry (DSC) | |
| CD58 variant | Tm (° C.) | Tmonset (° C.) | Tm (° C.) |
| CD58 Full ECD | 59.5 | 48.8 | 65.0 |
| CD58_IgV | 48.5 | 46.3 | 60.9 |
| IgV V45C_M105C | 48.5 | 43.9 | 66.8 |
| IgV V54C_G88C | 76.5 | 66.7 | 80.9 |
| IgV V45C_M114C | 63.5 | 49.6 | 72.5 |

Results of the affinity studies are shown in Table 27 below. Addition of stabilizing disulfide had no detrimental impact on the affinity or the binding stoichiometry.

TABLE 27

| CD58 variant | KD (uM) | n |
|---|---|---|
| CD58 Full ECD | 0.57 (±0.05) | 0.92 (±0.01) |
| CD58_IgV | 0.61 (±0.07) | 0.96 (±0.01) |
| IgV V45C_M105C | 0.88 (±0.06) | 0.97 (±0.01) |
| IgV V54C_G88C | 0.60 (±0.06) | 0.83 (±.0.01) |
| IgV V45C_M114C | 0.38 (±0.03) | 0.88 (±.0.01) |

### 8.3. Example 3: Combinations of CD3-TAA BBMs and CD2-TAA BBMs for Treating Liquid Tumors

To assess the effect of combining CD3-TAA BBMs with costimulatory BBMs engaging a tumor antigen and CD2 for targeting hematological malignancies, CD22 is used as a model system. CD3-CD22 BBMs are produced and tested alone and in combination with BBMs targeting (a) CD2 and the same epitope of CD22, (b) CD2 and different epitopes of CD22 and (c) CD2 and a second antigen, CD20, expressed on the same cells as CD22.

### 8.3.1. Materials and Methods

### 8.3.1.1. Production of BBMs in Knob-into-Holes Format

Gene synthesis for all BBM constructs are synthesized and codon optimized for expression in mammalian cells. For all constructs, anti-CD22 and anti-CD20 heavy chains are synthesized as fusions of the variable domains to constant hIgG1 domains containing T366S, L368A, and Y407V mutations for a hole to facilitate heterodimerization as well as silencing mutations. Light chain plasmids are synthesized fusing the variable light to the constant human kappa or lambda sequence sequence. For the anti-CD3 arms, these are produced as single chain variable fragments fused to constant hIgG1 domains containing a T366W mutation for the knob to facilitate heterodimerization as well as silencing mutations. Anti-CD2 arms are produced as either an anti-CD2 single chain variable fragment or a fragment of CD58 (CD58-6), the natural ligand for CD2, fused to constant hIgG1 domains containing a T366W mutation for a knob to facilitate heterodimerization as well as silencing mutations.

Amino acid sequences for components of the BBMs are shown in Table 28.

TABLE 28
BBM Amino Acid Sequences

| BBM | Chain | | SEQ |
|---|---|---|---|
| name | Description | | ID NO: |
| CD22_HA | First half | EVQLVESGGGLVKPGGSLKLSCAASGFAFSIYDMSWVRQTP | 1463 |
| 22-CD3 | antibody | EKRLEWVAYISSGGGTYYPDTVKGRFTISRDNAKNTLYLQMS | |
| Bispecific | (heavy chain) | SLKSEDTAMYYCARHSGYGTHWGVLFAYWGQGTLVTVSAA | |
| | (Fc sequence | STKGPSVFPLAPSSKSTSGGTAALGCLVKDYFPEPVTVSWN | |
| | not shown) | SGALTSGVHTFPAVLQSSGLYSLSSVVTVPSSSLGTQTYICN | |
| | | VNHKPSNTKVDKRVEPKSC | |
| | First half | EVQLVESGGGLVKPGGSLKLSCAASGFAFSIYDMSWVRQTP | 1464 |
| | antibody | EKRLEWVAYISSGGGTYYPDTVKGRFTISRDNAKNTLYLQMS | |
| | (heavy chain) | SLKSEDTAMYYCARHSGYGTHWGVLFAYWGQGTLVTVSAA | |
| | (includes Fc | STKGPSVFPLAPSSKSTSGGTAALGCLVKDYFPEPVTVSWN | |
| | sequence) | SGALTSGVHTFPAVLQSSGLYSLSSVVTVPSSSLGTQTYICN | |
| | | VNHKPSNTKVDKRVEPKSCDKTHTCPPCPAPELLGGPSVFL | |
| | | FPPKPKDTLMISRTPEVTCVVVAVSHEDPEVKFNWYVDGVE | |
| | | VHNAKTKPREEQYASTYRVVSVLTVLHQDWLNGKEYKCKVS | |
| | | NKALAAPIEKTISKAKGQPREPQVCTLPPSREEMTKNQVSLS | |
| | | CAVKGFYPSDIAVEWESNGQPENNYKTTPPVLDSDGSFFLV | |
| | | SKLTVDKSRWQQGNVFSCSVMHEALHNHYTQKSLSLSPGK | |
| | First half | DIQMTQTTSSLSASLGDRVTISCRASQDISNYLNWYQQKPDG | 1465 |
| | antibody | TVKLLIYYTSILHSGVPSRFSGSGSGTDYSLTISNLEQEDFATY | |
| | (light chain) | FCQQGNTLPWTFGGGTKLEIKRTVAAPSVFIFPPSDEQLKSG | |
| | | TASVVCLLNNFYPREAKVQWKVDNALQSGNSQESVTEQDSK | |
| | | DSTYSLSSTLTLSKADYEKHKVYACEVTHQGLSSPVTKSFNR | |
| | | GEC | |
| | Second half | EVQLVESGGGLVQPGGSLKLSCAASGFTFNTYAMNWVRQA | 1466 |
| | antibody | SGKGLEWVGRIRSKYNNYATYYADSVKDRFTISRDDSKSTLY | |
| | (Fc sequence | LQMNSLKTEDTAVYYCVRHGNFGNSYVSWFAYWGQGTLVT | |
| | not shown) | VSSGGGGSGGGGSGGGGSGGGGSQAVVTQEPSLTVSPGG | |
| | | TVTLTCRSSTGAVTTSNYANWVQQKPGQAPRGLIGGTNKRA | |
| | | PWTPARFSGSLLGDKAALTLSGAQPEDEAEYFCALWYSNLW | |
| | | VFGGGTKLTVLGGGGS | |
| | Second half | EVQLVESGGGLVQPGGSLKLSCAASGFTFNTYAMNWVRQA | 1467 |
| | antibody | SGKGLEWVGRIRSKYNNYATYYADSVKDRFTISRDDSKSTLY | |
| | (includes Fc | LQMNSLKTEDTAVYYCVRHGNFGNSYVSWFAYWGQGTLVT | |
| | sequence) | VSSGGGGSGGGGSGGGGSGGGGSQAVVTQEPSLTVSPGG | |
| | | TVTLTCRSSTGAVTTSNYANWVQQKPGQAPRGLIGGTNKRA | |
| | | PWTPARFSGSLLGDKAALTLSGAQPEDEAEYFCALWYSNLW | |
| | | VFGGGTKLTVLGGGGSDKTHTCPPCPAPELLGGPSVFLFPP | |
| | | KPKDTLMISRTPEVTCVVVAVSHEDPEVKFNWYVDGVEVHN | |
| | | AKTKPREEQYASTYRVVSVLTVLHQDWLNGKEYKCKVSNKA | |
| | | LAAPIEKTISKAKGQPREPQVYTLPPCREEMTKNQVSLWCLV | |
| | | KGFYPSDIAVEWESNGQPENNYKTTPPVLDSDGSFFLYSKLT | |
| | | VDKSRWQQGNVFSCSVMHEALHNRYTQKSLSLSPGK | |
| CD22_65- | First half | EVQLQQSGPGLVKPSQTLSLTCAISGDSMLSNSDTWNWIRQ | 1468 |
| CD3 | antibody | SPSRGLEWLGRTYHRSTWYDDYASSVRGRVSINVDTSKNQ | |
| Bispecific | (heavy chain) | YSLQLNAVTPEDTGVYYCARVRLQDGNSWSDAFDVWGQGT | |
| | (Fc sequence | MVTVSSASTKGPSVFPLAPSSKSTSGGTAALGCLVKDYFPEP | |
| | not shown) | VTVSWNSGALTSGVHTFPAVLQSSGLYSLSSVVTVPSSSLGT | |
| | | QTYICNVNHKPSNTKVDKRVEPKSC | |
| | First half | EVQLQQSGPGLVKPSQTLSLTCAISGDSMLSNSDTWNWIRG | 1469 |
| | antibody | SPSRGLEWLGRTYHRSTWYDDYASSVRGRVSINVDTSKNQ | |
| | (heavy chain) | YSLQLNAVTPEDTGVYYCARVRLQDGNSWSDAFDVWGQGT | |
| | (includes Fc | MVTVSSASTKGPSVFPLAPSSKSTSGGTAALGCLVKDYFPEP | |
| | sequence) | VTVSWNSGALTSGVHTFPAVLQSSGLYSLSSVVTVPSSSLGT | |
| | | QTYICNVNHKPSNTKVDKRVEPKSCDKTHTCPPCPAPELLG | |
| | | GPSVFLFPPKPKDTLMISRTPEVTCVVVAVSHEDPEVKFNWY | |
| | | VDGVEVHNAKTKPREEQYASTYRVVSVLTVLHQDWLNGKEY | |
| | | KCKVSNKALAAPIEKTISKAKGQPREPQVCTLPPSREEMTKN | |
| | | QVSLSCAVKGFYPSDIAVEWESNGQPENNYKTTPPVLDSDG | |
| | | SFFLVSKLTVDKSRWQQGNVFSCSVMHEALHNHYTQKSLSL | |
| | | SPGK | |
| | First half | QSALTQPASASGSPGQSVTISCTGTSSDVGGYNYVSWYQQ | 1470 |
| | antibody | HPGKAPKLMIYDVSNRPSGVSNRFSGSKSGNTASLTISGLQA | |
| | (light chain) | EDEADYYCSSYTSSSTLYVFGTGTQLTVLGQPKAAPSVTLFP | |
| | | PSSEELQANKATLVCLISDFYPGAVTVAWKADSSPVKAGVET | |
| | | TTPSKQSNNKYAASSYLSLTPEQWKSHRSYSCQVTHEGSTV | |
| | | EKTVAPTECS | |
| | Second half | EVQLVESGGGLVQPGGSLKLSCAASGFTFNTYAMNWVRQA | 1466 |
| | antibody | SGKGLEWVGRIRSKYNNYATYYADSVKDRFTISRDDSKSTLY | |
| | (Fc sequence | LQMNSLKTEDTAVYYCVRHGNFGNSYVSWFAYWGQGTLVT | |
| | not shown) | VSSGGGGSGGGGSGGGGSGGGGSQAVVTQEPSLTVSPGG | |
| | | TVTLTCRSSTGAVTTSNYANWVQQKPGQAPRGLIGGTNKRA | |
| | | PWTPARFSGSLLGDKAALTLSGAQPEDEAEYFCALWYSNLW | |
| | | VFGGGTKLTVLGGGGS | |
| | Second half | EVQLVESGGGLVQPGGSLKLSCAASGFTFNTYAMNWVRQA | 1467 |
| | antibody | SGKGLEWVGRIRSKYNNYATYYADSVKDRFTISRDDSKSTLY | |
| | (includes Fc | LQMNSLKTEDTAVYYCVRHGNFGNSYVSWFAYWGQGTLVT | |
| | sequence) | VSSGGGGSGGGGSGGGGSGGGGSQAVVTQEPSLTVSPGG | |
| | | TVTLTCRSSTGAVTTSNYANWVQQKPGQAPRGLIGGTNKRA | |
| | | PWTPARFSGSLLGDKAALTLSGAQPEDEAEYFCALWYSNLW | |
| | | VFGGGTKLTVLGGGGSDKTHTCPPCPAPELLGGPSVFLFPP | |
| | | KPKDTLMISRTPEVTCVVVAVSHEDPEVKFNWYVDGVEVHN | |
| | | AKTKPREEQYASTYRVVSVLTVLHQDWLNGKEYKCKVSNKA | |
| | | LAAPIEKTISKAKGQPREPQVYTLPPCREEMTKNQVSLWCLV | |
| | | KGFYPSDIAVEWESNGQPENNYKTTPPVLDSDGSFFLYSKLT | |
| | | VDKSRWQQGNVFSCSVMHEALHNRYTQKSLSLSPGK | |
| CD22_M9 | First half | QVQLQQSGPGLVKPSQTLSLTCAISGDSVSSNSAAWNWIRQ | 1471 |
| 71-CD3 | antibody | SPSRGLEWLGRTYYRSKWYNDYAVSVKSRITINPDTSKNQF | |
| Bispecific | (heavy chain) | SLQLNSVTPEDTAVYYCAREVTGDLEDAFDIWGQGTMVTVS | |
| | (Fc sequence | SASTKGPSVFPLAPSSKSTSGGTAALGCLVKDYFPEPVTVS | |
| | not shown) | WNSGALTSGVHTFPAVLQSSGLYSLSSWTVPSSSLGTQTYI | |
| | | CNVNHKPSNTKVDKRVEPKSC | |
| | First half | QVQLQQSGPGLVKPSQTLSLTCAISGDSVSSNSAAWNWIRQ | 1472 |
| | antibody | SPSRGLEWLGRTYYRSKWYNDYAVSVKSRITINPDTSKNQF | |
| | (heavy chain) | SLQLNSVTPEDTAVYYCAREVTGDLEDAFDIWGQGTMVTVS | |
| | (includes Fc | SASTKGPSVFPLAPSSKSTSGGTAALGCLVKDYFPEPVTVS | |
| | sequence) | WNSGALTSGVHTFPAVLQSSGLYSLSSVVTVPSSSLGTQTYI | |
| | | CNVNHKPSNTKVDKRVEPKSCDKTHTCPPCPAPELLGGPSV | |
| | | FLFPPKPKDTLMISRTPEVTCVVVAVSHEDPEVKFNWYVDGV | |
| | | EVHNAKTKPREEQYASTYRVVSVLTVLHQDWLNGKEYKCKV | |
| | | SNKALAAPIEKTISKAKGQPREPQVCTLPPSREEMTKNQVSL | |
| | | SCAVKGFYPSDIAVEWESNGQPENNYKTTPPVLDSDGSFFL | |
| | | VSKLTVDKSRWQQGNVFSCSVMHEALHNHYTQKSLSLSPG | |
| | | K | |
| | First half | DIQMTQSPSSLSASVGDRVTITCRASQTIWSYLNWYQQRPG | 1473 |
| | antibody | KAPNLLIYAASSLQSGVPSRFSGRGSGTDFTLTISSLQAEDFA | |
| | (light chain) | TYYCQQSYSIPQTFGQGTKLEIKRTVAAPSVFIFPPSDEQLKS | |
| | | GTASVVCLLNNFYPREAKVQWKVDNALQSGNSQESVTEQD | |
| | | SKDSTYSLSSTLTLSKADYEKHKVYACEVTHQGLSSPVTKSF | |
| | | NRGEC | |
| | Second half | EVQLVESGGGLVQPGGSLKLSCAASGFTFNTYAMNWVRQA | 1466 |
| | antibody | SGKGLEWVGRIRSKYNNYATYYADSVKDRFTISRDDSKSTLY | |
| | (Fc sequence | LQMNSLKTEDTAVYYCVRHGNFGNSYVSWFAYWGQGTLVT | |
| | not shown) | VSSGGGGSGGGGSGGGGSGGGGSQAVVTQEPSLTVSPGG | |
| | | TVTLTCRSSTGAVTTSNYANWVQQKPGQAPRGLIGGTNKRA | |
| | | PWTPARFSGSLLGDKAALTLSGAQPEDEAEYFCALWYSNLW | |
| | | VFGGGTKLTVLGGGGS | |
| | Second half | EVQLVESGGGLVQPGGSLKLSCAASGFTFNTYAMNWVRQA | 1467 |
| | antibody | SGKGLEWVGRIRSKYNNYATYYADSVKDRFTISRDDSKSTLY | |
| | (includes Fc | LQMNSLKTEDTAVYYCVRHGNFGNSYVSWFAYWGQGTLVT | |
| | sequence) | VSSGGGGSGGGGSGGGGSGGGGSQAVVTQEPSLTVSPGG | |
| | | TVTLTCRSSTGAVTTSNYANWVQQKPGQAPRGLIGGTNKRA | |
| | | PWTPARFSGSLLGDKAALTLSGAQPEDEAEYFCALWYSNLW | |
| | | VFGGGTKLTVLGGGGSDKTHTCPPCPAPELLGGPSVFLFPP | |
| | | KPKDTLMISRTPEVTCVVVAVSHEDPEVKFNWYVDGVEVHN | |
| | | AKTKPREEQYASTYRVVSVLTVLHQDWLNGKEYKCKVSNKA | |
| | | LAAPIEKTISKAKGQPREPQVYTLPPCREEMTKNQVSLWCLV | |
| | | KGFYPSDIAVEWESNGQPENNYKTTPPVLDSDGSFFLYSKLT | |
| | | VDKSRWQQGNVFSCSVMHEALHNRYTQKSLSLSPGK | |
| CD22_HA | First half | EVQLVESGGGLVKPGGSLKLSCAASGFAFSIYDMSWVRQTP | 1463 |
| 22-CD58 | antibody | EKRLEWVAYISSGGGTYYPDTVKGRFTISRDNAKNTLYLQMS | |
| Bispecific | (heavy chain) | SLKSEDTAMYYCARHSGYGTHWGVLFAYWGQGTLVTVSAA | |
| | (Fc sequence | STKGPSVFPLAPSSKSTSGGTAALGCLVKDYFPEPVTVSWN | |
| | not shown) | SGALTSGVHTFPAVLQSSGLYSLSSVVTVPSSSLGTQTYICN | |
| | | VNHKPSNTKVDKRVEPKSC | |
| | First half | EVQLVESGGGLVKPGGSLKLSCAASGFAFSIYDMSWVRQTP | 1464 |
| | antibody | EKRLEWVAYISSGGGTYYPDTVKGRFTISRDNAKNTLYLQMS | |
| | (heavy chain) | SLKSEDTAMYYCARHSGYGTHWGVLFAYWGQGTLVTVSAA | |
| | (includes Fc | STKGPSVFPLAPSSKSTSGGTAALGCLVKDYFPEPVTVSWN | |
| | sequence) | SGALTSGVHTFPAVLQSSGLYSLSSVVTVPSSSLGTQTYICN | |
| | | VNHKPSNTKVDKRVEPKSCDKTHTCPPCPAPELLGGPSVFL | |
| | | FPPKPKDTLMISRTPEVTCVVVAVSHEDPEVKFNWYVDGVE | |
| | | VHNAKTKPREEQYASTYRVVSVLTVLHQDWLNGKEYKCKVS | |
| | | NKALAAPIEKTISKAKGQPREPQVCTLPPSREEMTKNQVSLS | |
| | | CAVKGFYPSDIAVEWESNGQPENNYKTTPPVLDSDGSFFLV | |
| | | SKLTVDKSRWQQGNVFSCSVMHEALHNHYTQKSLSLSPGK | |
| | First half | DIQMTQTTSSLSASLGDRVTISCRASQDISNYLNWYQQKPDG | 1465 |
| | antibody | TVKLLIYYTSILHSGVPSRFSGSGSGTDYSLTISNLEQEDFATY | |
| | (light chain) | FCQQGNTLPWTFGGGTKLEIKRTVAAPSVFIFPPSDEQLKSG | |
| | | TASVVCLLNNFYPREAKVQWKVDNALQSGNSQESVTEQDSK | |
| | | DSTYSLSSTLTLSKADYEKHKVYACEVTHQGLSSPVTKSFNR | |
| | | GEC | |
| | Second half | SQQIYGVYGNVTFHVPSNVPLKEVLWKKQKDKVAELENSE | 1474 |
| | antibody | FRAFSSFKNRVYLDTVSGSLTIYNLTSSDEDEYEMESPNITDT | |
| | (Fc sequence | MKFFLYVLESGGGGS | |
| | not shown) | | |
| | Second half | SQQIYGVVYGNVTFHVPSNVPLKEVLWKKQKDKVAELENSE | 1475 |
| | antibody | FRAFSSFKNRVYLDTVSGSLTIYNLTSSDEDEYEMESPNITDT | |
| | (includes Fc | MKFFLYVLESGGGGSDKTHTCPPCPAPELLGGPSVFLFPPK | |
| | sequence) | PKDTLMISRTPEVTCVVVAVSHEDPEVKFNWYVDGVEVHNA | |
| | | KTKPREEQYASTYRVVSVLTVLHQDWLNGKEYKCKVSNKAL | |
| | | AAPIEKTISKAKGQPREPQVYTLPPCREEMTKNQVSLWCLVK | |
| | | GFYPSDIAVEWESNGQPENNYKTTPPVLDSDGSFFLYSKLTV | |
| | | DKSRWQQGNVFSCSVMHEALHNRYTQKSLSLSPGK | |
| CD22_65- | First half | EVQLQQSGPGLVKPSQTLSLTCAISGDSMLSNSDTWNWIRQ | 1468 |
| CD58 | antibody | SPSRGLEWLGRTYHRSTWYDDYASSVRGRVSINVDTSKNQ | |
| Bispecific | (heavy chain) | YSLQLNAVTPEDTGVYYCARVRLQDGNSWSDAFDVWGQGT | |
| | (Fc sequence | MVTVSSASTKGPSVFPLAPSSKSTSGGTAALGCLVKDYFPEP | |
| | not shown) | VTVSWNSGALTSGVHTFPAVLQSSGLYSLSSVVTVPSSSLGT | |
| | | QTYIONVNHKPSNTKVDKRVEPKSC | |
| | First half | EVQLQQSGPGLVKPSQTLSLTCAISGDSMLSNSDTWNWIRQ | 1469 |
| | antibody | SPSRGLEWLGRTYHRSTWYDDYASSVRGRVSINVDTSKNQ | |
| | (heavy chain) | YSLQLNAVTPEDTGVYYCARVRLQDGNSWSDAFDVWGQGT | |
| | (includes Fc | MVTVSSASTKGPSVFPLAPSSKSTSGGTAALGCLVKDYFPEP | |
| | sequence) | VTVSWNSGALTSGVHTFPAVLQSSGLYSLSSVVTVPSSSLGT | |
| | | QTYICNVNHKPSNTKVDKRVEPKSCDKTHTCPPCPAPELLG | |
| | | GPSVFLFPPKPKDTLMISRTPEVTCVVVAVSHEDPEVKFNWY | |
| | | VDGVEVHNAKTKPREEQYASTYRVVSVLTVLHQDWLNGKEY | |
| | | KCKVSNKALAAPIEKTISKAKGQPREPQVCTLPPSREEMTKN | |
| | | QVSLSCAVKGFYPSDIAVEWESNGQPENNYKTTPPVLDSDG | |
| | | SFFLVSKLTVDKSRWQQGNVFSCSVMHEALHNHYTQKSLSL | |
| | | SPGK | |
| | First half | QSALTQPASASGSPGQSVTISCTGTSSDVGGYNYVSWYQQ | 1470 |
| | antibody | HPGKAPKLMIYDVSNRPSGVSNRFSGSKSGNTASLTISGLQA | |
| | (light chain) | EDEADYYCSSYTSSSTLYVFGTGTQLTVLGQPKAAPSVTLFP | |
| | | PSSEELQANKATLVCLISDFYPGAVTVAWKADSSPVKAGVET | |
| | | TTPSKQSNNKYAASSYLSLTPEQWKSHRSYSCQVTHEGSTV | |
| | | EKTVAPTECS | |
| | Second half | SQQIYGVVYGNVTFHVPSNVPLKEVLWKKQKDKVAELENSE | 1474 |
| | antibody | FRAFSSFKNRVYLDTVSGSLTIYNLTSSDEDEYEMESPNITDT | |
| | (Fc sequence | MKFFLYVLESGGGGS | |
| | not shown) | | |
| | Second half | SQQIYGVVYGNVTFHVPSNVPLKEVLWKKQKDKVAELENSE | 1475 |
| | antibody | FRAFSSFKNRVYLDTVSGSLTIYNLTSSDEDEYEMESPNITDT | |
| | (includes Fc | MKFFLYVLESGGGGSDKTHTCPPCPAPELLGGPSVFLFPPK | |
| | sequence) | PKDTLMISRTPEVTCVVVAVSHEDPEVKFNWYVDGVEVHNA | |
| | | KTKPREEQYASTYRVVSVLTVLHQDWLNGKEYKCKVSNKAL | |
| | | AAPIEKTISKAKGQPREPQVYTLPPCREEMTKNQVSLWCLVK | |
| | | GFYPSDIAVEWESNGQPENNYKTTPPVLDSDGSFFLYSKLTV | |
| | | DKSRWQQGNVFSCSVMHEALHNRYTQKSLSLSPGK | |
| CD22_M9 | First half | QVQLQQSGPGLVKPSQTLSLTCAISGDSVSSNSAAWNWIRQ | 1471 |
| 71-CD58 | antibody | SPSRGLEWLGRTYYRSKWYNDYAVSVKSRITINPDTSKNQF | |
| Bispecific | (heavy chain) | SLQLNSVTPEDTAVYYCAREVTGDLEDAFDIWGQGTMVTVS | |
| | (Fc sequence | SASTKGPSVFPLAPSSKSTSGGTAALGCLVKDYFPEPVTVS | |
| | not shown) | WNSGALTSGVHTFPAVLQSSGLYSLSSVVTVPSSSLGTQTYI | |
| | | CNVNHKPSNTKVDKRVEPKSC | |
| | First half | QVQLQQSGPGLVKPSQTLSLTCAISGDSVSSNSAAWNWIRQ | 1472 |
| | antibody | SPSRGLEWLGRTYYRSKWYNDYAVSVKSRITINPDTSKNQF | |
| | (heavy chain) | SLQLNSVTPEDTAVYYCAREVTGDLEDAFDIWGQGTMVTVS | |
| | (includes Fc | SASTKGPSVFPLAPSSKSTSGGTAALGCLVKDYFPEPVTVS | |
| | sequence) | WNSGALTSGVHTFPAVLQSSGLYSLSSVVTVPSSSLGTQTYI | |
| | | CNVNHKPSNTKVDKRVEPKSCDKTHTCPPCPAPELLGGPSV | |
| | | FLFPPKPKDTLMISRTPEVTCVVVAVSHEDPEVKFNWYVDGV | |
| | | EVHNAKTKPREEQYASTYRVVSVLTVLHQDWLNGKEYKCKV | |
| | | SNKALAAPIEKTISKAKGQPREPQVCTLPPSREEMTKNQVSL | |
| | | SCAVKGFYPSDIAVEWESNGQPENNYKTTPPVLDSDGSFFL | |
| | | VSKLTVDKSRWQQGNVFSCSVMHEALHNHYTQKSLSLSPG | |
| | | K | |
| | First half | DIQMTQSPSSLSASVGDRVTITCRASQTIWSYLNWYQQRPG | 1473 |
| | antibody | KAPNLLIYAASSLQSGVPSRFSGRGSGTDFTLTISSLQAEDFA | |
| | (light chain) | TYYCQQSYSIPQTFGQGTKLEIKRTVAAPSVFIFPPSDEQLKS | |
| | | GTASVVCLLNNFYPREAKVQWKVDNALQSGNSQESVTEQD | |
| | | SKDSTYSLSSTLTLSKADYEKHKVYACEVTHQGLSSPVTKSF | |
| | | NRGEC | |
| | Second half | SQQIYGVVYGNVTFHVPSNVPLKEVLWKKQKDKVAELENSE | 1474 |
| | antibody | FRAFSSFKNRVYLDTVSGSLTIYNLTSSDEDEYEMESPNITDT | |
| | (Fc sequence | MKFFLYVLESGGGGS | |
| | not shown) | | |
| | Second half | SQQIYGVVYGNVTFHVPSNVPLKEVLWKKQKDKVAELENSE | 1475 |
| | antibody | FRAFSSFKNRVYLDTVSGSLTIYNLTSSDEDEYEMESPNITDT | |
| | (includes Fc | MKFFLYVLESGGGGSDKTHTCPPCPAPELLGGPSVFLFPPK | |
| | sequence) | PKDTLMISRTPEVTCVVVAVSHEDPEVKFNWYVDGVEVHNA | |
| | | KTKPREEQYASTYRVVSVLTVLHQDWLNGKEYKCKVSNKAL | |
| | | AAPIEKTISKAKGQPREPQVYTLPPCREEMTKNQVSLWCLVK | |
| | | GFYPSDIAVEWESNGQPENNYKTTPPVLDSDGSFFLYSKLTV | |
| | | DKSRWQQGNVFSCSVMHEALHNRYTQKSLSLSPGK | |
| CD22_HA | First half | EVQLVESGGGLVKPGGSLKLSCAASGFAFSIYDMSWVRQTP | 1463 |
| 22 | antibody | EKRLEWVAYISSGGGTYYPDTVKGRFTISRDNAKNTLYLQMS | |
| CD2_2- | (heavy chain) | SLKSEDTAMYYCARHSGYGTHWGVLFAYWGQGTLVTVSAA | |
| Bispecific | (Fc sequence | STKGPSVFPLAPSSKSTSGGTAALGCLVKDYFPEPVTVSWN | |
| | not shown) | SGALTSGVHTFPAVLQSSGLYSLSSVVTVPSSSLGTQTYICN | |
| | | VNHKPSNTKVDKRVEPKSC | |
| | First half | EVQLVESGGGLVKPGGSLKLSCAASGFAFSIYDMSWVRQTP | 1464 |
| | antibody | EKRLEWVAYISSGGGTYYPDTVKGRFTISRDNAKNTLYLQMS | |
| | (heavy chain) | SLKSEDTAMYYCARHSGYGTHWGVLFAYWGQGTLVTVSAA | |
| | (includes Fc | STKGPSVFPLAPSSKSTSGGTAALGCLVKDYFPEPVTVSWN | |
| | sequence) | SGALTSGVHTFPAVLQSSGLYSLSSVVTVPSSSLGTQTYICN | |
| | | VNHKPSNTKVDKRVEPKSCDKTHTCPPCPAPELLGGPSVFL | |
| | | FPPKPKDTLMISRTPEVTCVVVAVSHEDPEVKFNWYVDGVE | |
| | | VHNAKTKPREEQYASTYRVVSVLTVLHQDWLNGKEYKCKVS | |
| | | NKALAAPIEKTISKAKGQPREPQVCTLPPSREEMTKNQVSLS | |
| | | CAVKGFYPSDIAVEWESNGQPENNYKTTPPVLDSDGSFFLV | |
| | | SKLTVDKSRWQQGNVFSCSVMHEALHNHYTQKSLSLSPGK | |
| | First half | DIQMTQTTSSLSASLGDRVTISCRASQDISNYLNWYQQKPDG | 1465 |
| | antibody | TVKLLIYYTSILHSGVPSRFSGSGSGTDYSLTISNLEQEDFATY | |
| | (light chain) | FCQQGNTLPWTFGGGTKLEIKRTVAAPSVFIFPPSDEQLKSG | |
| | | TASVVCLLNNFYPREAKVQWKVDNALQSGNSQESVTEQDSK | |
| | | DSTYSLSSTLTLSKADYEKHKVYACEVTHQGLSSPVTKSFNR | |
| | | GEC | |
| | Second half | QVQLVQSGAEVQRPGASVKVSCKASGYIFTEYYMYWVRQA | 1476 |
| | antibody | PGQGLELVGRIDPEDGSIDYVEKFKKKVTLTADTSSSTAYME | |
| | (Fc sequence | LSSLTSDDTAVYYCARGKFNYRFAYWGQGTLVTVSSGGGG | |
| | not shown) | SGGGGSGGGGSGGGGSDVVMTQSPPSLLVTLGQPASISCR | |
| | | SSQSLLHSSGNTYLNWLLQRPGQSPQPLIYLVSKLESGVPDR | |
| | | FSGSGSGTDFTLKISGVEAEDVGVYYCMQFTHYPYTFGQGT | |
| | | KLEIKGGGGS | |
| | Second half | QVQLVQSGAEVQRPGASVKVSCKASGYIFTEYYMYWVRQA | 1477 |
| | antibody | PGQGLELVGRIDPEDGSIDYVEKFKKKVTLTADTSSSTAYME | |
| | (includes Fc | LSSLTSDDTAVYYCARGKFNYRFAYWGQGTLVTVSSGGGG | |
| | sequence) | SGGGGSGGGGSGGGGSDVVMTQSPPSLLVTLGQPASISCR | |
| | | SSQSLLHSSGNTYLNWLLQRPGQSPQPLIYLVSKLESGVPDR | |
| | | FSGSGSGTDFTLKISGVEAEDVGVYYCMQFTHYPYTFGQGT | |
| | | KLEIKGGGGSDKTHTCPPCPAPELLGGPSVFLFPPKPKDTLM | |
| | | ISRTPEVTCVVVAVSHEDPEVKFNWYVDGVEVHNAKTKPRE | |
| | | EQYASTYRVVSVLTVLHQDWLNGKEYKCKVSNKALAAPIEKT | |
| | | ISKAKGQPREPQVYTLPPCREEMTKNQVSLWCLVKGFYPSDI | |
| | | AVEWESNGQPENNYKTTPPVLDSDGSFFLYSKLTVDKSRWQ | |
| | | QGNVFSCSVMHEALHNRYTQKSLSLSPGK | |
| CD22_65- | First half | EVQLQQSGPGLVKPSQTLSLTCAISGDSMLSNSDTWNWIRQ | 1468 |
| CD2_2 | antibody | SPSRGLEWLGRTYHRSTWYDDYASSVRGRVSINVDTSKNQ | |
| Bispecific | (heavy chain) | YSLQLNAVTPEDTGVYYCARVRLQDGNSWSDAFDVWGQGT | |
| | (Fc sequence | MVTVSSASTKGPSVFPLAPSSKSTSGGTAALGCLVKDYFPEP | |
| | not shown) | VTVSWNSGALTSGVHTFPAVLQSSGLYSLSSVVTVPSSSLGT | |
| | | QTYICNVNHKPSNTKVDKRVEPKSC | |
| | First half | EVQLQQSGPGLVKPSQTLSLTCAISGDSMLSNSDTWNWIRQ | 1469 |
| | antibody | SPSRGLEWLGRTYHRSTWYDDYASSVRGRVSINVDTSKNQ | |
| | (heavy chain) | YSLQLNAVTPEDTGVYYCARVRLQDGNSWSDAFDVWGQGT | |
| | (includes Fc | MVTVSSASTKGPSVFPLAPSSKSTSGGTAALGCLVKDYFPEP | |
| | sequence) | VTVSWNSGALTSGVHTFPAVLQSSGLYSLSSVVTVPSSSLGT | |
| | | QTYICNVNHKPSNTKVDKRVEPKSCDKTHTCPPCPAPELLG | |
| | | GPSVFLFPPKPKDTLMISRTPEVTCVVVAVSHEDPEVKFNWY | |
| | | VDGVEVHNAKTKPREEQYASTYRVVSVLTVLHQDWLNGKEY | |
| | | KCKVSNKALAAPIEKTISKAKGQPREPQVCTLPPSREEMTKN | |
| | | QVSLSCAVKGFYPSDIAVEWESNGQPENNYKTTPPVLDSDG | |
| | | SFFLVSKLTVDKSRWQQGNVFSCSVMHEALHNHYTQKSLSL | |
| | | SPGK | |
| | First half | QSALTQPASASGSPGQSVTISCTGTSSDVGGYNYVSWYQQ | 1470 |
| | antibody | HPGKAPKLMIYDVSNRPSGVSNRFSGSKSGNTASLTISGLQA | |
| | (light chain) | EDEADYYCSSYTSSSTLYVFGTGTQLTVLGQPKAAPSVTLFP | |
| | | PSSEELQANKATLVCLISDFYPGAVTVAWKADSSPVKAGVET | |
| | | TTPSKQSNNKYAASSYLSLTPEQWKSHRSYSCQVTHEGSTV | |
| | | EKTVAPTECS | |
| | Second half | QVQLVQSGAEVQRPGASVKVSCKASGYIFTEYYMYWVRQA | 1476 |
| | antibody | PGQGLELVGRIDPEDGSIDYVEKFKKKVTLTADTSSSTAYME | |
| | (Fc sequence | LSSLTSDDTAVYYCARGKFNYRFAYWGQGTLVTVSSGGGG | |
| | not shown) | SGGGGSGGGGSGGGGSDVVMTQSPPSLLVTLGQPASISCR | |
| | | SSQSLLHSSGNTYLNWLLQRPGQSPQPLIYLVSKLESGVPDR | |
| | | FSGSGSGTDFTLKISGVEAEDVGVYYCMQFTHYPYTFGQGT | |
| | | KLEIKGGGGS | |
| | Second half | QVQLVQSGAEVQRPGASVKVSCKASGYIFTEYYMYWVRQA | 1477 |
| | antibody | PGQGLELVGRIDPEDGSIDYVEKFKKKVTLTADTSSSTAYME | |
| | (includes Fc | LSSLTSDDTAVYYCARGKFNYRFAYWGQGTLVTVSSGGGG | |
| | sequence) | SGGGGSGGGGSGGGGSDVVMTQSPPSLLVTLGQPASISCR | |
| | | SSQSLLHSSGNTYLNWLLQRPGQSPQPLIYLVSKLESGVPDR | |
| | | FSGSGSGTDFTLKISGVEAEDVGVYYCMQFTHYPYTFGQGT | |
| | | KLEIKGGGGSDKTHTCPPCPAPELLGGPSVFLFPPKPKDTLM | |
| | | ISRTPEVTCVVVAVSHEDPEVKFNWYVDGVEVHNAKTKPRE | |
| | | EQYASTYRVVSVLTVLHQDWLNGKEYKCKVSNKALAAPIEKT | |
| | | ISKAKGQPREPQVYTLPPCREEMTKNQVSLWCLVKGFYPSDI | |
| | | AVEWESNGQPENNYKTTPPVLDSDGSFFLYSKLTVDKSRWQ | |
| | | QGNVFSCSVMHEALHNRYTQKSLSLSPGK | |
| CD22_M9 | First half | QVQLQQSGPGLVKPSQTLSLTCAISGDSVSSNSAAWNWIRQ | 1471 |
| 71-CD2_2 | antibody | SPSRGLEWLGRTYYRSKWYNDYAVSVKSRITINPDTSKNQF | |
| Bispecific | (heavy chain) | SLQLNSVTPEDTAVYYCAREVTGDLEDAFDIWGQGTMVTVS | |
| | (Fc sequence | SASTKGPSVFPLAPSSKSTSGGTAALGCLVKDYFPEPVTVS | |
| | not shown) | WNSGALTSGVHTFPAVLQSSGLYSLSSVVTVPSSSLGTQTYI | |
| | | CNVNHKPSNTKVDKRVEPKSC | |
| | First half | QVQLQQSGPGLVKPSQTLSLTCAISGDSVSSNSAAWNWIRQ | 1472 |
| | antibody | SPSRGLEWLGRTYYRSKWYNDYAVSVKSRITINPDTSKNQF | |
| | (heavy chain) | SLQLNSVTPEDTAVYYCAREVTGDLEDAFDIWGQGTMVTVS | |
| | (includes Fc | SASTKGPSVFPLAPSSKSTSGGTAALGCLVKDYFPEPVTVS | |
| | sequence) | WNSGALTSGVHTFPAVLQSSGLYSLSSVVTVPSSSLGTQTYI | |
| | | CNVNHKPSNTKVDKRVEPKSCDKTHTCPPCPAPELLGGPSV | |
| | | FLFPPKPKDTLMISRTPEVTCVVVAVSHEDPEVKFNWYVDGV | |
| | | EVHNAKTKPREEQYASTYRVVSVLTVLHQDWLNGKEYKCKV | |
| | | SNKALAAPIEKTISKAKGQPREPQVCTLPPSREEMTKNQVSL | |
| | | SCAVKGFYPSDIAVEWESNGQPENNYKTTPPVLDSDGSFFL | |
| | | VSKLTVDKSRWQQGNVFSCSVMHEALHNHYTQKSLSLSPG | |
| | | K | |
| | First half | DIQMTQSPSSLSASVGDRVTITCRASQTIWSYLNWYQQRPG | 1473 |
| | antibody | KAPNLLIYAASSLQSGVPSRFSGRGSGTDFTLTISSLQAEDFA | |
| | (light chain) | TYYCQQSYSIPQTFGQGTKLEIKRTVAAPSVFIFPPSDEQLKS | |
| | | GTASVVCLLNNFYPREAKVQWKVDNALQSGNSQESVTEQD | |
| | | SKDSTYSLSSTLTLSKADYEKHKVYACEVTHQGLSSPVTKSF | |
| | | NRGEC | |
| | Second half | QVQLVQSGAEVQRPGASVKVSCKASGYIFTEYYMYWVRQA | 1476 |
| | antibody | PGQGLELVGRIDPEDGSIDYVEKFKKKVTLTADTSSSTAYME | |
| | (Fc sequence | LSSLTSDDTAVYYCARGKFNYRFAYWGQGTLVTVSSGGGG | |
| | not shown) | SGGGGSGGGGSGGGGSDVVMTQSPPSLLVTLGQPASISCR | |
| | | SSQSLLHSSGNTYLNWLLQRPGQSPQPLIYLVSKLESGVPDR | |
| | | FSGSGSGTDFTLKISGVEAEDVGVYYCMQFTHYPYTFGQGT | |
| | | KLEIKGGGGS | |
| | Second half | QVQLVQSGAEVQRPGASVKVSCKASGYIFTEYYMYWVRQA | 1477 |
| | antibody | PGQGLELVGRIDPEDGSIDYVEKFKKKVTLTADTSSSTAYME | |
| | (includes Fc | LSSLTSDDTAVYYCARGKFNYRFAYWGQGTLVTVSSGGGG | |
| | sequence) | SGGGGSGGGGSGGGGSDVVMTQSPPSLLVTLGQPASISCR | |
| | | SSQSLLHSSGNTYLNWLLQRPGQSPQPLIYLVSKLESGVPDR | |
| | | FSGSGSGTDFTLKISGVEAEDVGVYYCMQFTHYPYTFGQGT | |
| | | KLEIKGGGGSDKTHTCPPCPAPELLGGPSVFLFPPKPKDTLM | |
| | | ISRTPEVTCVVVAVSHEDPEVKFNWYVDGVEVHNAKTKPRE | |
| | | EQYASTYRVVSVLTVLHQDWLNGKEYKCKVSNKALAAPIEKT | |
| | | ISKAKGQPREPQVYTLPPCREEMTKNQVSLWCLVKGFYPSDI | |
| | | AVEWESNGQPENNYKTTPPVLDSDGSFFLYSKLTVDKSRWQ | |
| | | QGNVFSCSVMHEALHNRYTQKSLSLSPGK | |
| CD20_1- | First half | QVQLQQPGAELVKPGASVKMSCKASGYTFTSYNMHWVKQT | 1478 |
| CD58 | antibody | PGRGLEWIGAIYPGNGDTSYNQKFKGKATLTADKSSSTAYM | |
| Bispecific | (heavy chain) | QLSSLTSEDSAVYYCARSTYYGGDWYFNVWGAGTTVTVSA | |
| | (Fc sequence | ASTKGPSVFPLAPSSKSTSGGTAALGCLVKDYFPEPVTVSW | |
| | not shown) | NSGALTSGVHTFPAVLQSSGLYSLSSWTVPSSSLGTQTYIC | |
| | | NVNHKPSNTKVDKRVEPKSC | |
| | First half | QVQLQQPGAELVKPGASVKMSCKASGYTFTSYNMHWVKQT | 1479 |
| | antibody | PGRGLEWIGAIYPGNGDTSYNQKFKGKATLTADKSSSTAYM | |
| | (heavy chain) | QLSSLTSEDSAVYYCARSTYYGGDWYFNVWGAGTTVTVSA | |
| | (includes Fc | ASTKGPSVFPLAPSSKSTSGGTAALGCLVKDYFPEPVTVSW | |
| | sequence) | NSGALTSGVHTFPAVLQSSGLYSLSSVVTVPSSSLGTQTYIC | |
| | | NVNHKPSNTKVDKRVEPKSCDKTHTCPPCPAPELLGGPSVF | |
| | | LFPPKPKDTLMISRTPEVTCVVVAVSHEDPEVKFNWVDGVE | |
| | | VHNAKTKPREEQYASTYRVVSVLTVLHQDWNGKEYKCKVS | |
| | | NKALAAPIEKTISKAKGQPREPQVCTLPPSREEMTKNQVSLS | |
| | | CAVKGFYPSDIAVEWESNGQPENNYKTTPPVLDSDGSFFLV | |
| | | SKLTVDKSRWQQGNVFSCSVMHEALHNHYTQKSLSLSPGK | |
| | First half | QIVLSQSPAILSASPGEKVTMTCRASSSVSYIHWFQQKPGSS | 1480 |
| | antibody | PKPWIYATSNLASGVPVRFSGSGSGTSYSLTISRVEAEDAAT | |
| | (light chain) | YYCQQWTSNPPTFGGGTKLEIKRTVAAPSVFIFPPSDEQLKS | |
| | | GTASVVCLLNNFYPREAKVQWKVDNALQSGNSQESVTEQD | |
| | | SKDSTYSLSSTLTLSKADYEKHKVYACEVTHQGLSSPVTKSF | |
| | | NRGEC | |
| | Second half | SQQIYGVVYGNVTFHVPSNVPLKEVLWKKQKDKVAELENSE | 1474 |
| | antibody | FRAFSSFKNRVYLDTVSGSLTIYNLTSSDEDEYEMESPNITDT | |
| | (Fc sequence | MKFFLYVLESGGGGS | |
| | not shown) | | |
| | Second half | SQQIYGVVYGNVTFHVPSNVPLKEVLWKKQKDKVAELENSE | 1475 |
| | antibody | FRAFSSFKNRVYLDTVSGSLTIYNLTSSDEDEYEMESPNITDT | |
| | (includes Fc | MKFFLYVLESGGGGSDKTHTCPPCPAPELLGGPSVFLFPPK | |
| | sequence) | PKDTLMISRTPEVTCVVVAVSHEDPEVKFNWYVDGVEVHNA | |
| | | KTKPREEQYASTYRVVSVLTVLHQDWLNGKEYKCKVSNKAL | |
| | | AAPIEKTISKAKGQPREPQWTLPPCREEMTKNQVSLWCLVK | |
| | | GFYPSDIAVEWESNGQPENNYKTTPPVLDSDGSFFLYSKLTV | |
| | | DKSRWQQGNVFSCSVMHEALHNRYTQKSLSLSPGK | |
| CD20_1- | First half | QVQLQQPGAELVKPGASVKMSCKASGYTFTSYNMHWVKQT | 1478 |
| CD2_2 | antibody | PGRGLEWIGAIYPGNGDTSYNQKFKGKATLTADKSSSTAYM | |
| Bispecific | (heavy chain) | QLSSLTSEDSAVYYCARSTYYGGDWYFNVWGAGTTVTVSA | |
| | (Fc sequence | ASTKGPSVFPLAPSSKSTSGGTAALGCLVKDYFPEPVTVSW | |
| | not shown) | NSGALTSGVHTFPAVLQSSGLYSLSSVVTVPSSSLGTQTYIC | |
| | | NVNHKPSNTKVDKRVEPKSC | |
| | First half | QVQLQQPGAELVKPGASVKMSCKASGYTFTSYNMHWVKQT | 1479 |
| | antibody | PGRGLEWIGAIYPGNGDTSYNQKFKGKATLTADKSSSTAYM | |
| | (heavy chain) | QLSSLTSEDSAVYYCARSTYYGGDWYFNVWGAGTTVTVSA | |
| | (includes Fc | ASTKGPSVFPLAPSSKSTSGGTAALGCLVKDYFPEPVTVSW | |
| | sequence) | NSGALTSGVHTFPAVLQSSGLYSLSSVVTVPSSSLGTQTYIC | |
| | | NVNHKPSNTKVDKRVEPKSCDKTHTCPPCPAPELLGGPSVF | |
| | | LFPPKPKDTLMISRTPEVTCVVVAVSHEDPEVKFNWYVDGVE | |
| | | VHNAKTKPREEQYASTYRVVSVLTVLHQDWLNGKEYKCKVS | |
| | | NKALAAPIEKTISKAKGQPREPQVCTLPPSREEMTKNQVSLS | |
| | | CAVKGFYPSDIAVEWESNGQPENNYKTTPPVLDSDGSFFLV | |
| | | SKLTVDKSRWQQGNVFSCSVMHEALHNHYTQKSLSLSPGK | |
| | First half | QIVLSQSPAILSASPGEKVTMTCRASSSVSYIHWFQQKPGSS | 1480 |
| | antibody | PKPWIYATSNLASGVPVRFSGSGSGTSYSLTISRVEAEDAAT | |
| | (light chain) | YYCQQWTSNPPTFGGGTKLEIKRTVAAPSVFIFPPSDEQLKS | |
| | | GTASVVCLLNNFYPREAKVQWKVDNALQSGNSQESVTEQD | |
| | | SKDSTYSLSSTLTLSKADYEKHKVYACEVTHQGLSSPVTKSF | |
| | | NRGEC | |
| | Second half | QVQLVQSGAEVQRPGASVKVSCKASGYIFTEYYMYWVRQA | 1476 |
| | antibody | PGQGLELVGRIDPEDGSIDYVEKFKKKVTLTADTSSSTAYME | |
| | (Fc sequence | LSSLTSDDTAVYYCARGKFNYRFAYWGQGTLVTVSSGGGG | |
| | not shown) | SGGGGSGGGGSGGGGSDVVMTQSPPSLLVTLGQPASISCR | |
| | | SSQSLLHSSGNTYLNWLLQRPGQSPQPLIYLVSKLESGVPDR | |
| | | FSGSGSGTDFTLKISGVEAEDVGVYYCMQFTHYPYTFGQGT | |
| | | KLEIKGGGGS | |
| | Second half | QVQLVQSGAEVQRPGASVKVSCKASGYIFTEYYMYWVRQA | 1477 |
| | antibody | PGQGLELVGRIDPEDGSIDYVEKFKKKVTLTADTSSSTAYME | |
| | (includes Fc | LSSLTSDDTAVYYCARGKFNYRFAYWGQGTLVTVSSGGGG | |
| | sequence) | SGGGGSGGGGSGGGGSDVVMTQSPPSLLVTLGQPASISCR | |
| | | SSQSLLHSSGNTYLNWLLQRPGQSPQPLIYLVSKLESGVPDR | |
| | | FSGSGSGTDFTLKISGVEAEDVGVYYCMQFTHYPYTFGQGT | |
| | | KLEIKGGGGSDKTHTCPPCPAPELLGGPSVFLFPPKPKDTLM | |
| | | ISRTPEVTCVVVAVSHEDPEVKFNWYVDGVEVHNAKTKPRE | |
| | | EQYASTYRVVSVLTVLHQDWLNGKEYKCKVSNKALAAPIEKT | |
| | | ISKAKGQPREPQVYTLPPCREEMTKNQVSLWCLVKGFYPSDI | |
| | | AVEWESNGQPENNYKTTPPVLDSDGSFFLYSKLTVDKSRWQ | |
| | | QGNVFSCSVMHEALHNRYTQKSLSLSPGK | |

### 8.3.1.2. Expression and Purification

BBMs are expressed transiently by co-transfection of the respective chains in HEK293 cells. Briefly, transfection is performed using PEI as transfection reagent. For small scale (<5L) transfections, cells are grown in shake flasks on an orbital shaker (115 rpm) in a humidified incubator (85%) at 5% CO2). Light and heavy chain plasmids for tumor antigen arms are combined with scFv plasmids with PEI at a final ratio of 1 DNA:3 PEI. 1 mg/L culture of plasmid is used for transfection at 2.0 million cells/mL serum media. After 5 days of expression, the BBMs are harvested by clarification of the media via centrifugation and filtration. Purification is performed via anti-CH1 affinity batch binding (CaptureSelect IgG-CH1 Affinity Matrix, Thermo-Fisher Scientific, Waltham, Mass., USA) or Protein A (rProteinA Sepharose, Fast flow, GE Healthcare, Uppsala, Sweden) batch binding using 1 ml resin/100 mL supernatant. The protein is allowed to bind for a minimum of 2 hours with gentle mixing, and the supernatant is loaded onto a gravity filtration column. The resin is washed with 20-50 CV of PBS. Antibody is eluted with 20 CV of 50 mM citrate, 90 mM NaCl pH 3.2. 50 mM sucrose. The eluted IgG protein is adjusted to pH 5.5 with 1 M sodium citrate 50 mM sucrose. If the antibody contains aggregates, preparative size exclusion chromatography is performed using a Hi Load 16/60 Superdex 200 grade column (GE Healthcare Life Sciences, Uppsala, Sweden) as a final polishing step. To confirm that the identity of the proteins expressed matches the predicted masses for the primary amino acid sequences, BBMs are analyzed by high-performance liquid chromatography coupled to mass spectrometry.

### 8.3.1.3. Re-Directed T Cell Cytotoxicity Assay (Quantitative Luciferase Assay)

Purified BBMs are analyzed alone and in combination for their potential to induce T cell-mediated apoptosis in tumor target cells.

The purified BBMs are compared across multiple donor effector cells. Briefly, doubly CD22-CD20-expressing (e.g. Raji or Daudi) target cells are engineered to overexpress firefly luciferase. Cells are harvested and resuspended in RPMI medium (Invitrogen #11875-093) with 10% FBS. 5 000 target cells per well are plated in a flat-bottom 384-well plate. Human pan T effector cells are isolated via negative selection (Stemcell Technologies #17951) from cryopreserved PBMC that are separated from a leukopak (Hemacare #PB001F-1) by Ficoll density gradient centrifugation. Purified T cells are then added to the plate to obtain varying final E:T ratios. Co-cultured cells are incubated with a serial dilution of all constructs and controls. For normalization, average maximum luminescence refers to target cells co-incubated with effector cells, but without any test construct. After an incubation of either 48, 72 or 96 hr at 37° C., 5% CO2, OneGlo luciferase substrate (Promega #E6120) is added to the plate. Luminescence is measured on an Envision plate reader after a 10 minute incubation. Percent specific lysis is calculated using the following equation:

Specific lysis (%)=(1−(sample luminescence/average maximum luminescence))*100

### 8.3.2. Cytokine Release Assay

Purified BBMs are analyzed alone and in combination for their ability to induce T cell-mediated de novo secretion of cytokines in the presence of tumor target cells.

Briefly, doubly expressing CD22-CD20 (e.g. Raji or Daudi) target cells are harvested and resuspended in RPMI medium with 10% FBS. 20 000 target cells per well are plated in a flat-bottom 96-well plate. Human pan T effector cells are isolated via MACS negative selection from cryopreserved PBMC then added to the plate to obtain varying final E:T ratios. Co-cultured cells are incubated with a serial dilution of all constructs and controls. After an incubation of 24 hr at 37° C., 5% CO2 the supernatants are harvested by centrifugation at 300×g for 5 min for subsequent analysis.

A multiplexed ELISA is performed according to the manufacturer's instructions using a V-PLEX Proinflammatory Panel 1 Kit (MesoScale Discovery #K15049D).

### 8.3.3. Results

The combinations of CD3-CD22 targeting BBMs and CD2-CD22 targeting BBMs show an additive amount of T cell mediated apoptosis in the re-directed T cell cytotoxicity assay as compared to the MBMs alone. The combinations of CD3-TAA targeting BBMs and CD2-TAA targeting BBMs show an additive amount of cytokine release in the cytokine release assay as compared to the MBMs alone.

### 8.4. Example 4: Combinations of CD3-TAA BBMs and CD2-TAA BBMs for Treating Solid Tumors

To assess the effect of combining CD3-TAA BBMs with costimulatory BBMs engaging a tumor antigen and CD2 for targeting solid tumors, mesothelin (MSLN) is used as a model system. CD3 BBMs are produced and tested alone and in combination with BBMs targeting CD2 and the same epitope of MSLN and different epitopes of MSLN.

### 8.4.1. Materials and Methods

### 8.4.1.1. Production of BBMs in Knob-into-Holes Format

BBM constructs are synthesized and codon optimized for expression in mammalian cells. For all constructs, anti-MSLN heavy chains are synthesized as fusions of the variable domains to constant hIgG1 domains containing T366S, L368A, and Y407V mutations for a hole to facilitate heterodimerization as well as silencing mutations. Light chain plasmids are synthesized fusing the variable light to the constant human kappa sequence. For the anti-CD3 arms, these are produced as single chain variable fragments fused to constant hIgG1 domains containing a T366W mutation for a knob to facilitate heterodimerization as well as silencing mutations. Anti-CD2 arms are produced as either an anti-CD2 single chain variable fragment or a fragment of CD58 (CD58-6), the natural ligand for CD2, fused to constant hIgG1 domains containing a T366W mutation for a knob to facilitate heterodimerization as well as silencing mutations.

Amino acid sequences for components of the BBMs are shown in Table 29.

TABLE 29
BBM Amino Acid Sequences

| | Chain | | SEQ ID |
|---|---|---|---|
| BBM Name | Description | Amino acid sequence | NO: |
| MSLN_SS1- | First half | QVQLQQSGPELEKPGASVKISCKASGYSFTGYTMNWV | 1481 |
| CD3-16nM | antibody | KQSHGKSLEWIGLITPYNGASSYNQKFRGKATLTVDKSS | |
| Bispecific | (heavy chain) | STAYMDLLSLTSEDSAVYFCARGGYDGRGFDYWGQGT | |
| | (Fc sequence | TVTVSSASTKGPSVFPLAPSSKSTSGGTAALGCLVKDYF | |
| | not shown) | PEPVTVSWNSGALTSGVHTFPAVLQSSGLYSLSSVVTV | |
| | | PSSSLGTQTYICNVNHKPSNTKVDKRVEPKSC | |
| | First half | QVQLQQSGPELEKPGASVKISCKASGYSFTGYTMNWV | 1482 |
| | antibody | KQSHGKSLEWIGLITPYNGASSYNQKFRGKATLTVDKSS | |
| | (heavy chain) | STAYMDLLSLTSEDSAVYFCARGGYDGRGFDYWGQGT | |
| | (includes Fc | TVTVSSASTKGPSVFPLAPSSKSTSGGTAALGCLVKDYF | |
| | sequence) | PEPVTVSWNSGALTSGVHTFPAVLQSSGLYSLSSVVTV | |
| | | PSSSLGTQTYICNVNHKPSNTKVDKRVEPKSCDKTHTC | |
| | | PPCPAPELLGGPSVFLFPPKPKDTLMISRTPEVTCVVVA | |
| | | VSHEDPEVKFNWYVDGVEVHNAKTKPREEQYASTYRV | |
| | | VSVLTVLHQDWLNGKEYKCKVSNKALAAPIEKTISKAKG | |
| | | QPREPQVCTLPPSREEMTKNQVSLSCAVKGFYPSDIAV | |
| | | EWESNGQPENNYKTTPPVLDSDGSFFLVSKLTVDKSR | |
| | | WQQGNVFSCSVMHEALHNHYTQKSLSLSPGK | |
| | First half | DIELTQSPAIMSASPGEKVTMTCSASSSVSYMHWWYQQK | 1483 |
| | antibody | SGTSPKRWIYDTSKLASGVPGRFSGSGSGNSYSLTISS | |
| | (light chain) | VEAEDDATYYCQQWSGYPLTFGAGTKLEIKRTVAAPSV | |
| | | FIFPPSDEQLKSGTASVVCLLNNFYPREAKVQWKVDNA | |
| | | LQSGNSQESVTEQDSKDSTYSLSSTLTLSKADYEKHKV | |
| | | YACEVTHQGLSSPVTKSFNRGEC | |
| | Second half | EVQLVESGGGLVQPGGSLKLSCAASGFTFNTYAMNWV | 1466 |
| | antibody | RQASGKGLEWVGRIRSKYNNYATYYADSVKDRFTISRD | |
| | (Fc sequence | DSKSTLYLQMNSLKTEDTAVYYCVRHGNFGNSYVSWF | |
| | not shown) | AYWGQGTLVTVSSGGGGSGGGGSGGGGSGGGGSQA | |
| | | VVTQEPSLTVSPGGTVTLTCRSSTGAVTTSNYANWVQQ | |
| | | KPGQAPRGLIGGTNKRAPWTPARFSGSLLGDKAALTLS | |
| | | GAQPEDEAEYFCALWYSNLWVFGGGTKLTVLGGGGS | |
| | Second half | VQLVESGGGLVQPGGSLKLSCAASGFTFNTYAMNWVR | 1484 |
| | antibody | QASGKGLEWVGRIRSKYNNYATYYADSVKDRFTISRDD | |
| | (includes Fc | SKSTLYLQMNSLKTEDTAVYYCVRHGNFGNSYVSWFAY | |
| | sequence) | WGQGTLVTVSSGGGGSGGGGSGGGGSGGGGSQAVV | |
| | | TQEPSLTVSPGGTVTLTCRSSTGAVTTSNYANWVQQKP | |
| | | GQAPRGLIGGTNKRAPWTPARFSGSLLGDKAALTLSGA | |
| | | QPEDEAEYFCALWYSNLWVFGGGTKLTVLGGGGSDKT | |
| | | HTCPPCPAPELLGGPSVFLFPPKPKDTLMISRTPEVTCV | |
| | | VVAVSHEDPEVKFNWYVDGVEVHNAKTKPREEQYAST | |
| | | YRVVSVLTVLHQDWLNGKEYKCKVSNKALAAPIEKTISK | |
| | | AKGQPREPQVYTLPPCREEMTKNQVSLWCLVKGFYPS | |
| | | DIAVEWESNGQPENNYKTTPPVLDSDGSFFLYSKLTVD | |
| | | KSRWQQGNVFSCSVMHEALHNRYTQKSLSLSPGK | |
| MSLN_M5- | First half | QVQLVQSGAEVEKPGASVKVSCKASGYTFTDYYMHWV | 1485 |
| CD3-16nM | antibody | RQAPGQGLEWMGWINPNSGGTNYAQKFQGRVTMTRD | |
| Bispecific | (heavy chain) | TSISTAYMELSRLRSDDTAVYYCASGWDFDYWGQGTLV | |
| | (Fc sequence | TVSSASTKGPSVFPLAPSSKSTSGGTAALGCLVKDYFPE | |
| | not shown) | PVTVSWNSGALTSGVHTFPAVLQSSGLYSLSSVVTVPS | |
| | | SSLGTQTYICNVNHKPSNTKVDKRVEPKSC | |
| | First half | QVQLVQSGAEVEKPGASVKVSCKASGYTFTDYYMHWV | 1426 |
| | antibody | RQAPGQGLEWMGWINPNSGGTNYAQKFQGRVTMTRD | |
| | (heavy chain) | TSISTAYMELSRLRSDDTAVYYCASGWDFDYWGQGTLV | |
| | (includes Fc | TVSSASTKGPSVFPLAPSSKSTSGGTAALGCLVKDYFPE | |
| | sequence) | PVTVSWNSGALTSGVHTFPAVLQSSGLYSLSSVVTVPS | |
| | | SSLGTQTYICNVNHKPSNTKVDKRVEPKSCDKTHTCPP | |
| | | CPAPELLGGPSVFLFPPKPKDTLMISRTPEVTCVVVAVS | |
| | | HEDPEVKFNWYVDGVEVHNAKTKPREEQYASTYRVVS | |
| | | VLTVLHQDWLNGKEYKCKVSNKALAAPIEKTISKAKGQP | |
| | | REPQVCTLPPSREEMTKNQVSLSCAVKGFYPSDIAVEW | |
| | | ESNGQPENNYKTTPPVLDSDGSFFLVSKLTVDKSRWQ | |
| | | QGNVFSCSVMHEALHNHYTQKSLSLSPGK | |
| | First half | DIVMTQSPSSLSASVGDRVTITCRASQSIRYYLSWYQQK | 1427 |
| | antibody | PGKAPKLLIYTASILQNGVPSRFSGSGSGTDFTLTISSLQ | |
| | (light chain) | PEDFATYYCLQTYTTPDFGPGTKVEIKRTVAAPSVFIFPP | |
| | | SDEQLKSGTASVVCLLNNFYPREAKVQWKVDNALQSG | |
| | | NSQESVTEQDSKDSTYSLSSTLTLSKADYEKHKVYACE | |
| | | VTHQGLSSPVTKSFNRGEC | |
| | Second half | EVQLVESGGGLVQPGGSLKLSCAASGFTFNTYAMNWV | 1466 |
| | antibody | RQASGKGLEWVGRIRSKYNNYATYYADSVKDRFTISRD | |
| | (Fc sequence | DSKSTLYLQMNSLKTEDTAVYYCVRHGNFGNSYVSWF | |
| | not shown) | AYWGQGTLVTVSSGGGGSGGGGSGGGGSGGGGSQA | |
| | | VVTQEPSLTVSPGGTVTLTCRSSTGAVTTSNYANWVQQ | |
| | | KPGQAPRGLIGGTNKRAPWTPARFSGSLLGDKAALTLS | |
| | | GAQPEDEAEYFCALWYSNLWVFGGGTKLTVLGGGGS | |
| | Second half | EVQLVESGGGLVQPGGSLKLSCAASGFTFNTYAMNWV | 1467 |
| | antibody | RQASGKGLEWVGRIRSKYNNYATYYADSVKDRFTISRD | |
| | (includes Fc | DSKSTLYLQMNSLKTEDTAVYYCVRHGNFGNSYVSWF | |
| | sequence) | AYWGQGTLVTVSSGGGGSGGGGSGGGGSGGGGSQA | |
| | | VVTQEPSLTVSPGGTVTLTCRSSTGAVTTSNYANWVQQ | |
| | | KPGQAPRGLIGGTNKRAPWTPARFSGSLLGDKAALTLS | |
| | | GAQPEDEAEYFCALWYSNLWVFGGGTKLTVLGGGGSD | |
| | | KTHTCPPCPAPELLGGPSVFLFPPKPKDTLMISRTPEVT | |
| | | CVVVAVSHEDPEVKFNWYVDGVEVHNAKTKPREEQYA | |
| | | STYRVVSVLTVLHQDWLNGKEYKCKVSNKALAAPIEKTI | |
| | | SKAKGQPREPQVYTLPPCREEMTKNQVSLWCLVKGFY | |
| | | PSDIAVEWESNGQPENNYKTTPPVLDSDGSFFLYSKLT | |
| | | VDKSRWQQGNVFSCSVMHEALHNRYTQKSLSLSPGK | |
| MSLN_SS1- | First half | QVQLQQSGPELEKPGASVKISCKASGYSFTGYTMNWV | 1481 |
| CD58- | antibody | KQSHGKSLEWIGLITPYNGASSYNQKFRGKATLTVDKSS | |
| Bispecific | (heavy chain) | STAYMDLLSLTSEDSAVYFCARGGYDGRGFDYWGQGT | |
| | (Fc sequence | TVTVSSASTKGPSVFPLAPSSKSTSGGTAALGCLVKDYF | |
| | not shown) | PEPVTVSWNSGALTSGVHTFPAVLQSSGLYSLSSVVTV | |
| | | PSSSLGTQTYICNVNHKPSNTKVDKRVEPKSC | |
| | First half | QVQLQQSGPELEKPGASVKISCKASGYSFTGYTMNWV | 1482 |
| | antibody | KQSHGKSLEWIGLITPYNGASSYNQKFRGKATLTVDKSS | |
| | (heavy chain) | STAYMDLLSLTSEDSAVYFCARGGYDGRGFDYWGQGT | |
| | (includes Fc | TVTVSSASTKGPSVFPLAPSSKSTSGGTAALGCLVKDYF | |
| | sequence) | PEPVTVSWNSGALTSGVHTFPAVLQSSGLYSLSSWTV | |
| | | PSSSLGTQTYICNVNHKPSNTKVDKRVEPKSCDKTHTC | |
| | | PPCPAPELLGGPSVFLFPPKPKDTLMISRTPEVTCVVVA | |
| | | VSHEDPEVKFNWYVDGVEVHNAKTKPREEQYASTYRV | |
| | | VSVLTVLHQDWLNGKEYKCKVSNKALAAPIEKTISKAKG | |
| | | QPREPQVCTLPPSREEMTKNQVSLSCAVKGFYPSDIAV | |
| | | EWESNGQPENNYKTTPPVLDSDGSFFLVSKLTVDKSR | |
| | | WQQGNVFSCSVMHEALHNHYTQKSLSLSPGK | |
| | First half | DIELTQSPAIMSASPGEKVTMTCSASSSVSYMHWYQQK | 1483 |
| | antibody | SGTSPKRWIYDTSKLASGVPGRFSGSGSGNSYSLTISS | |
| | (light chain) | VEAEDDATYYCQQWSGYPLTFGAGTKLEIKRTVAAPSV | |
| | | FIFPPSDEQLKSGTASVVCLLNNFYPREAKVQWKVDNA | |
| | | LQSGNSQESVTEQDSKDSTYSLSSTLTLSKADYEKHKV | |
| | | YACEVTHQGLSSPVTKSFNRGEC | |
| | Second half | SQQIYGVVYGNVTFHVPSNVPLKEVLWKKQKDKVAELE | 1474 |
| | antibody | NSEFRAFSSFKNRVYLDTVSGSLTIYNLTSSDEDEYEME | |
| | (Fc sequence | SPNITDTMKFFLYVLESGGGGS | |
| | not shown) | | |
| | Second half | SQQIYGVVYGNVTFHVPSNVPLKEVLWKKQKDKVAELE | 1475 |
| | antibody | NSEFRAFSSFKNRVYLDTVSGSLTIYNLTSSDEDEYEME | |
| | (includes Fc | SPNITDTMKFFLYVLESGGGGSDKTHTCPPCPAPELLG | |
| | sequence) | GPSVFLFPPKPKDTLMISRTPEVTCVVVAVSHEDPEVKF | |
| | | NWYVDGVEVHNAKTKPREEQYASTYRVVSVLTVLHQD | |
| | | WLNGKEYKCKVSNKALAAPIEKTISKAKGQPREPQVYTL | |
| | | PPCREEMTKNQVSLWCLVKGFYPSDIAVEWESNGQPE | |
| | | NNYKTTPPVLDSDGSFFLYSKLTVDKSRWQQGNVFSCS | |
| | | VMHEALHNRYTQKSLSLSPGK | |
| MSLN_M5- | First half | QVQLVQSGAEVEKPGASVKVSCKASGYTFTDYYMHWV | 1485 |
| CD58 | antibody | RQAPGQGLEWMGWINPNSGGTNYAQKFQGRVTMTRD | |
| Bispecific | (heavy chain) | TSISTAYMELSRLRSDDTAVYYCASGWDFDYWGQGTLV | |
| | (Fc sequence | TVSSASTKGPSVFPLAPSSKSTSGGTAALGCLVKDYFPE | |
| | not shown) | PVTVSWNSGALTSGVHTFPAVLQSSGLYSLSSVVTVPS | |
| | | SSLGTQTYICNVNHKPSNTKVDKRVEPKSC | |
| | First half | QVQLVQSGAEVEKPGASVKVSCKASGYTFTDYYMHWV | 1426 |
| | antibody | RQAPGQGLEWMGWINPNSGGTNYAQKFQGRVTMTRD | |
| | (heavy chain) | TSISTAYMELSRLRSDDTAVYYCASGWDFDYWGQGTLV | |
| | (includes Fc | TVSSASTKGPSVFPLAPSSKSTSGGTAALGCLVKDYFPE | |
| | sequence) | PVTVSWNSGALTSGVHTFPAVLQSSGLYSLSSVVTVPS | |
| | | SSLGTQTYICNVNHKPSNTKVDKRVEPKSCDKTHTCPP | |
| | | CPAPELLGGPSVFLFPPKPKDTLMISRTPEVTCVVVAVS | |
| | | HEDPEVKFNWYVDGVEVHNAKTKPREEQYASTYRVVS | |
| | | VLTVLHQDWLNGKEYKCKVSNKALAAPIEKTISKAKGQP | |
| | | REPQVCTLPPSREEMTKNQVSLSCAVKGFYPSDIAVEW | |
| | | ESNGQPENNYKTTPPVLDSDGSFFLVSKLTVDKSRWQ | |
| | | QGNVFSCSVMHEALHNHYTQKSLSLSPGK | |
| | First half | DIVMTQSPSSLSASVGDRVTITCRASQSIRYYLSWYQQK | 1427 |
| | antibody | PGKAPKLLIYTASILQNGVPSRFSGSGSGTDFTLTISSLQ | |
| | (light chain) | PEDFATYYCLQTYTTPDFGPGTKVEIKRTVAAPSVFIFPP | |
| | | SDEQLKSGTASVVCLLNNFYPREAKVQWKVDNALQSG | |
| | | NSQESVTEQDSKDSTYSLSSTLTLSKADYEKHKVYACE | |
| | | VTHQGLSSPVTKSFNRGEC | |
| | Second half | SQQIYGVVYGNVTFHVPSNVPLKEVLWKKQKDKVAELE | 1474 |
| | antibody | NSEFRAFSSFKNRVYLDTVSGSLTIYNLTSSDEDEYEME | |
| | (Fc sequence | SPNITDTMKFFLYVLESGGGGS | |
| | not shown) | | |
| | Second half | SQQIYGVVYGNVTFHVPSNVPLKEVLWKKQKDKVAELE | 1475 |
| | antibody | NSEFRAFSSFKNRVYLDTVSGSLTIYNLTSSDEDEYEME | |
| | (includes Fc | SPNITDTMKFFLYVLESGGGGSDKTHTCPPCPAPELLG | |
| | sequence) | GPSVFLFPPKPKDTLMISRTPEVTCVVVAVSHEDPEVKF | |
| | | NWYVDGVEVHNAKTKPREEQYASTYRVVSVLTVLHQD | |
| | | WLNGKEYKCKVSNKALAAPIEKTISKAKGQPREPQVYTL | |
| | | PPCREEMTKNQVSLWCLVKGFYPSDIAVEWESNGQPE | |
| | | NNYKTTPPVLDSDGSFFLYSKLTVDKSRWQQGNVFSCS | |
| | | VMHEALHNRYTQKSLSLSPGK | |
| MSLN_SS1- | First half | QVQLQQSGPELEKPGASVKISCKASGYSFTGYTMNWV | 1481 |
| CD2_2 | antibody | KQSHGKSLEWIGLITPYNGASSYNQKFRGKATLTVDKSS | |
| Bispecific | (heavy chain) | STAYMDLLSLTSEDSAVYFCARGGYDGRGFDYWGQGT | |
| | (Fc sequence | TVTVSSASTKGPSVFPLAPSSKSTSGGTAALGCLVKDYF | |
| | not shown) | PEPVTVSWNSGALTSGVHTFPAVLQSSGLYSLSSVVTV | |
| | | PSSSLGTQTYICNVNHKPSNTKVDKRVEPKSC | |
| | First half | QVQLQQSGPELEKPGASVKISCKASGYSFTGYTMNWV | 1482 |
| | antibody | KQSHGKSLEWIGLITPYNGASSYNQKFRGKATLTVDKSS | |
| | (heavy chain) | STAYMDLLSLTSEDSAVYFCARGGYDGRGFDYWGQGT | |
| | (includes Fc | TVTVSSASTKGPSVFPLAPSSKSTSGGTAALGCLVKDYF | |
| | sequence) | PEPVTVSWNSGALTSGVHTFPAVLQSSGLYSLSSVVTV | |
| | | PSSSLGTQTYICNVNHKPSNTKVDKRVEPKSCDKTHTC | |
| | | PPCPAPELLGGPSVFLFPPKPKDTLMISRTPEVTCVVVA | |
| | | VSHEDPEVKFNWYVDGVEVHNAKTKPREEQYASTYRV | |
| | | VSVLTVLHQDWLNGKEYKCKVSNKALAAPIEKTISKAKG | |
| | | QPREPQVCTLPPSREEMTKNQVSLSCAVKGFYPSDIAV | |
| | | EWESNGQPENNYKTTPPVLDSDGSFFLVSKLTVDKSR | |
| | | WQQGNVFSCSVMHEALHNHYTQKSLSLSPGK | |
| | First half | DIELTQSPAIMSASPGEKVTMTCSASSSVSYMHWYQQK | 1483 |
| | antibody | SGTSPKRWIYDTSKLASGVPGRFSGSGSGNSYSLTISS | |
| | (light chain) | VEAEDDATYYCQQWSGYPLTFGAGTKLEIKRTVAAPSV | |
| | | FIFPPSDEQLKSGTASVVCLLNNFYPREAKVQWKVDNA | |
| | | LQSGNSQESVTEQDSKDSTYSLSSTLTLSKADYEKHKV | |
| | | YACEVTHQGLSSPVTKSFNRGEC | |
| | Second half | QVQLVQSGAEVQRPGASVKVSCKASGYIFTEYYMYWV | 1476 |
| | antibody | RQAPGQGLELVGRIDPEDGSIDYVEKFKKKVTLTADTSS | |
| | (Fc sequence | STAYMELSSLTSDDTAVYYCARGKFNYRFAYWGQGTLV | |
| | not shown) | TVSSGGGGSGGGGSGGGGSGGGGSDVVMTQSPPSLL | |
| | | VTLGQPASISCRSSQSLLHSSGNTYLNWLLQRPGQSPQ | |
| | | PLIYLVSKLESGVPDRFSGSGSGTDFTLKISGVEAEDVG | |
| | | VYYCMQFTHYPYTFGQGTKLEIKGGGGS | |
| | Second half | QVQLVQSGAEVQRPGASVKVSCKASGYIFTEYYMYWV | 1477 |
| | antibody | RQAPGQGLELVGRIDPEDGSIDYVEKFKKKVTLTADTSS | |
| | (includes Fc | STAYMELSSLTSDDTAVYYCARGKFNYRFAYWGQGTLV | |
| | sequence) | TVSSGGGGSGGGGSGGGGSGGGGSDVVMTQSPPSLL | |
| | | VTLGQPASISCRSSQSLLHSSGNTYLNWLLQRPGQSPQ | |
| | | PLIYLVSKLESGVPDRFSGSGSGTDFTLKISGVEAEDVG | |
| | | VYYCMQFTHYPYTFGQGTKLEIKGGGGSDKTHTCPPCP | |
| | | APELLGGPSVFLFPPKPKDTLMISRTPEVTCVVVAVSHE | |
| | | DPEVKFNWYVDGVEVHNAKTKPREEQYASTYRVVSVLT | |
| | | VLHQDWLNGKEYKCKVSNKALAAPIEKTISKAKGQPREP | |
| | | QVYTLPPCREEMTKNQVSLWCLVKGFYPSDIAVEWESN | |
| | | GQPENNYKTTPPVLDSDGSFFLYSKLTVDKSRWQQGN | |
| | | VFSCSVMHEALHNRYTQKSLSLSPGK | |
| MSLN_M5- | First half | QVQLVQSGAEVEKPGASVKVSCKASGYTFTDYYMHWV | 1485 |
| CD2_2 | antibody | RQAPGQGLEWMGWNPNSGGTNYAQKFQGRVTMTRD | |
| Bispecific | (heavy chain) | TSISTAYMELSRLRSDDTAVYYCASGWDFDYWGQGTLV | |
| | (Fc sequence | TVSSASTKGPSVFPLAPSSKSTSGGTAALGCLVKDYFPE | |
| | not shown) | PVTVSWNSGALTSGVHTFPAVLQSSGLYSLSSVVTVPS | |
| | | SSLGTQTYICNVNHKPSNTKVDKRVEPKSC | |
| | First half | QVQLVQSGAEVEKPGASVKVSCKASGYTFTDYYMHVW | 1426 |
| | antibody | RQAPGQGLEWMGWINPNSGGTNYAQKFQGRVTMTRD | |
| | (heavy chain) | TSISTAYMELSRLRSDDTAVYYCASGWDFDYWGQGTLV | |
| | (includes Fc | TVSSASTKGPSVFPLAPSSKSTSGGTAALGCLVKDYFPE | |
| | sequence) | PVTVSWNSGALTSGVHTFPAVLQSSGLYSLSSVVTVPS | |
| | | SSLGTQTYICNVNHKPSNTKVDKRVEPKSCDKTHTCPP | |
| | | CPAPELLGGPSVFLFPPKPKDTLMISRTPEVTCVVVVAVS | |
| | | HEDPEVKFNWYVDGVEVHNAKTKPREEQYASTYRVVS | |
| | | VLTVLHQDWLNGKEYKCKVSNKALAAPIEKTISKAKGQP | |
| | | REPQVCTLPPSREEMTKNQVSLSCAVKGFYPSDIAVEW | |
| | | ESNGQPENNYKTTPPVLDSDGSFFLVSKLTVDKSRWQ | |
| | | QGNVFSCSVMHEALHNHYTQKSLSLSPGK | |
| | First half | DIVMTQSPSSLSASVGDRVTITCRASQSIRYYLSWYQQK | 1427 |
| | antibody | PGKAPKLLIYTASILQNGVPSRFSGSGSGTDFTLTISSLQ | |
| | (light chain) | PEDFATYYCLQTYTTPDFGPGTKVEIKRTVAAPSVFIFPP | |
| | | SDEQLKSGTASVVCLLNNFYPREAKVQWKVDNALQSG | |
| | | NSQESVTEQDSKDSTYSLSSTLTLSKADYEKHKVYACE | |
| | | VTHQGLSSPVTKSFNRGEG | |
| | Second half | QVQLVQSGAEVQRPGASVKVSCKASGYIFTEYYMYWV | 1476 |
| | antibody | RQAPGQGLELVGRIDPEDGSIDYVEKFKKKVTLTADTSS | |
| | (Fc sequence | STAYMELSSLTSDDTAVYYCARGKFNYRFAYWGQGTLV | |
| | not shown) | TVSSGGGGSGGGGSGGGGSGGGGSDVVMTQSPPSLL | |
| | | VTLGQPASISCRSSQSLLHSSGNTYLNWLLQRPGQSPQ | |
| | | PLIYLVSKLESGVPDRFSGSGSGTDFTLKISGVEAEDVG | |
| | | VYYCMQFTHYPYTFGQGTKLEIKGGGGS | |
| | Second half | QVQLVQSGAEVQRPGASVKVSCKASGYIFTEYYMYWV | 1477 |
| | antibody | RQAPGQGLELVGRIDPEDGSIDYVEKFKKKVTLTADTSS | |
| | (includes Fc | STAYMELSSLTSDDTAVYYCARGKFNYRFAYWGQGTLV | |
| | sequence) | TVSSGGGGSGGGGSGGGGSGGGGSDVVMTQSPPSLL | |
| | | VTLGQPASISCRSSQSLLHSSGNTYLNWLLQRPGQSPQ | |
| | | PLIYLVSKLESGVPDRFSGSGSGTDFTLKISGVEAEDVG | |
| | | VYYCMQFTHYPYTFGQGTKLEIKGGGGSDKTHTCPPCP | |
| | | APELLGGPSVFLFPPKPKDTLMISRTPEVTCVVVAVSHE | |
| | | DPEVKFNWYVDGVEVHNAKTKPREEQYASTYRVVSVLT | |
| | | VLHQDWLNGKEYKCKVSNKALAAPIEKTISKAKGQPREP | |
| | | QVYTLPPCREEMTKNQVSLWCLVKGFYPSDIAVEWESN | |
| | | GQPENNYKTTPPVLDSDGSFFLYSKLTVDKSRWQQGN | |
| | | VFSCSVMHEALHNRYTQKSLSLSPGK | |

### 8.4.1.2. Expression and Purification

BBMs are expressed transiently by co-transfection of the respective chains in HEK293 cells. Briefly, transfection is performed using PEI as transfection reagent. For small scale (<5L) transfections, cells are grown in shake flasks on an orbital shaker (115 rpm) in a humidified incubator (85%) at 5% CO2). Light and heavy chain plasmids for tumor antigen arms are combined with scFv plasmids with PEI at a final ratio of 1 DNA:3 PEI. 1 mg/L culture of plasmid is used for transfection at 2.0 million cells/mL serum media. After 5 days of expression, the BBMs are harvested by clarification of the media via centrifugation and filtration. Purification is performed via anti-CH1 affinity batch binding (CaptureSelect IgG-CH1 Affinity Matrix, Thermo-Fisher Scientific, Waltham, Mass., USA) or Protein A (rProteinA Sepharose, Fast flow, GE Healthcare, Uppsala, Sweden) batch binding using 1 ml resin/100 mL supernatant. The protein is allowed to bind for a minimum of 2 hours with gentle mixing, and the supernatant is loaded onto a gravity filtration column. The resin is washed with 20-50 CV of PBS. Antibody is eluted with 20 CV of 50 mM citrate, 90 mM NaCl pH 3.2. 50 mM sucrose. The eluted IgG protein is adjusted to pH 5.5 with 1 M sodium citrate 50 mM sucrose. If the antibody contains aggregates, preparative size exclusion chromatography is performed using a Hi Load 16/60 Superdex 200 grade column (GE Healthcare Life Sciences, Uppsala, Sweden) as a final polishing step. To confirm that the identity of the proteins expressed matches the predicted masses for the primary amino acid sequences, BBMs are analyzed by high-performance liquid chromatography coupled to mass spectrometry.

### 8.4.1.3. Re-Directed T Cell Cytotoxicity Assay (Quantitative Luciferase Assay)

Purified BBMs are analyzed alone and in combination for their potential to induce T cell-mediated apoptosis in tumor target cells.

The purified BBMs are compared across multiple donor effector cells. Briefly, huMSLN-expressing (e.g. OVCAR8) target cells are engineered to overexpress firefly luciferase. Cells are harvested and resuspended in RPMI medium (Invitrogen #11875-093) with 10% FBS. 5 000 target cells per well are plated in a flat-bottom 384-well plate. Human pan T effector cells are isolated via negative selection (Stemcell Technologies #17951) from cryopreserved PBMC that are separated from a leukopak (Hemacare #PB001F-1) by Ficoll density gradient centrifugation. Purified T cells are added to the plate to obtain varying final E:T ratios. Co-cultured cells are incubated with a serial dilution of all constructs and controls. For normalization, average maximum luminescence refers to target cells co-incubated with effector cells, but without any test construct. After an incubation of either 48, 72 or 96 hr at 37° C., 5% CO2, OneGlo luciferase substrate (Promega #E6120) are added to the plate. Luminescence is measured on an Envision plate reader after a 10 minute incubation. Percent specific lysis is calculated using the following equation:

Specific lysis (%)=(1−(sample luminescence/average maximum luminescence))*100

### 8.4.2. Cytokine Release Assay

Purified BBMs are analyzed alone and in combination for their ability to induce T cell-mediated de novo secretion of cytokines in the presence of tumor target cells.

Briefly, huMSLN-expressing (e.g., OVCAR8) target cells are harvested and resuspended in RPMI medium with 10% FBS. 20 000 target cells per well are plated in a flat-bottom 96-well plate. Human pan T effector cells are isolated via MACS negative selection from cryopreserved PBMC then added to the plate to obtain varying final E:T ratios. Co-cultured cells are incubated with a serial dilution of all constructs and controls. After an incubation of 24 hr at 37° C., 5% CO2 the supernatants are harvested by centrifugation at 300×g for 5 min for subsequent analysis.

A multiplexed ELISA is performed according to the manufacturer's instructions using a V-PLEX Proinflammatory Panel 1 Kit (MesoScale Discovery #K15049D).

### 8.4.3. Results

The combinations of CD3-MSLN targeting BBMs and CD2-MSLN targeting BBMs show an additive amount of T cell mediated apoptosis in the re-directed T cell cytotoxicity assay as compared to the MBMs alone. The combinations of CD3-MSLN targeting BBMs and CD2-MSLN targeting BBMs show an additive amount of cytokine release in the cytokine release assay as compared to the MBMs alone.

### 8.5. Example 5: CD28 and CD2 Engagement Augments Anti-CD3 Antibody-Induced T Cell Proliferation and Cytokine Secretion

The effects of CD28 and CD2 engagement on T cell proliferation and cytokine secretion in the presence or absence of CD3 stimulation with an anti-CD3 antibody were evaluated.

### 8.5.1. Materials and Methods

High-binding plates (Corning 29444-456) were first coated with 100 μl PBS containing 0 μg/ml or 1.1 μg/ml anti-CD3 antibody (Thermo Fisher 16-0037-85) in PBS at 37° C. for 2 hours. The same plates were then washed with PBS for 3 times and coated with anti-CD28 antibody (Thermo Fisher 16-0289-85) or CD58-Fc protein (R&D system 10068-CD-050) in a serial dilution of 1:3 starting at 10 μg/ml. Human pan T cells were isolated via negative selection (Miltenyi 130-096-535) from cryopreserved PBMCs (one donor) that were separated from a leukopak (Hemacare #PB001F-1) by Ficoll density gradient centrifugation. 70,000 purified T cells in 200 μl culture media (RPMI medium (Invitrogen #11875-093) with 10% FBS) were then added to each well in the plates. Cells were incubated for 3 days at 37° C., 5% CO2. 25 μl of supernatants were harvested from each well for cytokine analysis. A multiplexed ELISA was performed according to the manufacturer's instructions using a human cytokine custom 3-plex 384 4-spot kit (MesoScale Discovery #N31IB-1). 100 μl Cell titer Glo substrate (Promega G7573) was added to each well. Luminescence was measured on an Envision plate reader after a 5 minute incubation.

### 8.5.2. Results

As shown in FIGS. 3A-3D, engagement of CD28 by plate-bound anti-CD28 antibody and engagement CD2 by plate-bound CD58-Fc protein augmented T cell proliferation (FIG. 3A) and cytokine secretion (IL2 (FIG. 3B), IFNg (FIG. 3C), and TNFa (FIG. 3D)) in the presence of CD3 stimulation (1.1 μg/ml anti-CD3 antibody). CD28 or CD2 engagement by plate-bound anti-CD28 antibody or CD58-Fc protein, respectively, did not augment T cell proliferation and cytokine secretion in the absence of CD3 stimulation (0 μg/ml anti-CD3 antibody).

### 8.6. Example 6: CD2×CD20 BBM Enhances CD3×CD19 BBM-Induced T Cell Activation in NFAT Jurkat Reporter Assay

The effect of a CD2×CD20 BBM on CD3×CD19 BBM-induced T cell activation in a NFAT Jurkat reporter assay was evaluated.

### 8.6.1. Materials and Methods

CD2×CD20 BBM and CD3×CD19 BBM sequences are set forth in Tables 30A-30B, respectively. To construct the CD2×CD20 BBM, an anti-CD2 scFv was fused to a first Fc region to provide a first half antibody, and an anti-CD20 Fab was fused to a second Fc region to provide a second half antibody. The first and second Fc regions included knob and hole mutations to promote heterodimer formation, and the second Fc region also included RF mutations to ease purification (Tustian et al., 2016, MAbs 8(4):828-838). The CD3×CD19 BBM was one-arm BBM having, in the N-terminal to C-terminal direction, an anti-CD19 Fab, an anti-CD3 scFv, and an Fc domain with knob and hole and silencing mutations.

The CD2×CD20 BBM and CD3×CD19 BBM were expressed in mammalian cells and purified before setting up a NFAT Jurkat reporter assay. Briefly, Jurkat cells were engineered to express a firefly luciferase reporter gene responding to NFAT transcription factor. Cells were harvested and resuspended in RPMI medium (Invitrogen #11875-093) with 10% FBS. 5,000 Karpas422 tumor cells and 15,000 Jurkat NFAT reporter cells were plated in each well of a flat-bottom 384-well plate. Co-cultured cells were incubated with serial dilutions of CD2×CD20 BBM (in the absence or presence of 0.8 nM CD3×CD19 BBM) or CD3×CD19 BBM (in the absence or presence of 20 nM CD2×CD20 BBM). After an incubation of 18 hr at 37° C., 5% CO2, Bright-Glo luciferase substrate (Promega #E2650) was added to the plate. Luminescence was measured on an Envision plate reader after a 5 minute incubation.

TABLE 30A
Amino acid sequences

| Construct | Chain | | SEQ |
|---|---|---|---|
| Name | Description | Amino Acid Sequence | ID NO: |
| CD2xCD20 | First Half | QVQLVQSGAEVQRPGASVKVSCKASGYIFTEYYMYWVRQ | 1428 |
| BBM | Antibody | APGQGLELVGRIDPEDGSIDYVEKFKKKVTLTADTSSSTAY | |
| | | MELSSLTSDDTAVYYCARGKFNYRFAYWGQGTLVTVSSG | |
| | | GGGSGGGGSGGGGSGGGGSDVMTQSPPSLLVTLGQPA | |
| | | SISCRSSQSLLHSSGNTYLNWLLQRPGQSPQPLIYLVSKLE | |
| | | SGVPDRFSGSGSGTDFTLKISGVEAEDVGVYYCMQFTHYP | |
| | | YTFGQGTKLEIKRGGGGSDKTHTCPPCPAPELLGGPSVFL | |
| | | FPPKPKDTLMISRTPEVTCVVVAVSHEDPEVKFNWYVDGV | |
| | | EVHNAKTKPREEQYASTYRVVSVLTVLHQDWLNGKEYKCK | |
| | | VSNKALAAPIEKTISKAKGQPREPQVYTLPPCREEMTKNQV | |
| | | SLWCLVKGFYPSDIAVEWESNGQPENNYKTTPPVLDSDGS | |
| | | FFLYSKLTVDKSRWQQGNVFSCSVMHEALHNHYTQKSLSL | |
| | | SPGK | |
| | Second Half | QVQLQQPGAELVKPGASVKMSCKASGYTFTSYNMHWVKQ | 1429 |
| | Antibody | TPGRGLEWIGAIYPGNGDTSYNQKFKGKATLTADKSSSTA | |
| | Heavy Chain | YMQLSSLTSEDSAVYYOARSTYYGGDWFNVWGAGTTVT | |
| | | VSSASTKGPSVFPLAPSSKSTSGGTAALGCLVKDYFPEPVT | |
| | | VSWNSGALTSGVHTFPAVLQSSGLYSLSSVVTVPSSSLGT | |
| | | QTYICNVNHKPSNTKVDKRVEPKSCDKTHTCPPCPAPELL | |
| | | GGPSVFLFPPKPKDTLMISRTPEVTCVVVVAVSHEDPEVKFN | |
| | | WYVDGVEVHNAKTKPREEQYASTYRVVSVLTVLHQDWLN | |
| | | GKEYKCKVSNKALAAPIEKTISKAKGQPREPQVCTLPPSRE | |
| | | EMTKNQVSLSCAVKGFYPSDIAVEWESNGQPENNYKTTPP | |
| | | VLDSDGSFFLVSKLTVDKSRWQQGNVFSCSVMHEALHNR | |
| | | FTQKSLSLSPGK | |
| | Second Half | QIVLSQSPAILSASPGEKVTMTCRASSSVSYIHWFQQKPGS | 1480 |
| | Antibody Light | SPKPWIYATSNLASGVPVRFSGSGSGTSYSLTISRVEAE | |
| | Chain | DAATYYCQQWTSNPPTFGGGTKLEIKRTVAAPSVFIFPPSD | |
| | | EQLKSGTASVVCLLNNFYPREAKVQWKVDNALQSGNSQE | |
| | | SVTEQDSKDSTYSLSSTLTLSKADYEKHKVYACEVTHQGLS | |
| | | SPVTKSFNRGEC | |

TABLE 30B
Amino acid sequences

| Construct | Chain | | SEQ |
|---|---|---|---|
| Name | Description | Amino Acid Sequence | ID NO: |
| CD3xCD19 | First Half | QVQLVQSGAEVKKPGASVKVSCKASGYTFTTYWIQWVRQ | 1430 |
| BBM | Antibody | APGQRLEWMGAVYPGDADTRYTQKFQGRVTLTADRSAST | |
| | Heavy Chain | AYMELSSLRSEDTAVYYCGRDAGLEYYALDYWGQGTLVTV | |
| | | SSASTKGPSVFPLAPSSKSTSGGTAALGCLVKDYFPEPVTV | |
| | | SWNSGALTSGVHTFPAVLQSSGLYSLSSVVTVPSSSLGTQ | |
| | | TYICNVNHKPSNTKVDKRVEPKSCGGGGSGGGGSEVQLV | |
| | | ESGGGLVQPGGSLKLSCAASGFTFNTYAMNWVRQASGKG | |
| | | LEWVGRIRSKYNNYATYYADSVKDRFTISRDDSKSTLYLQM | |
| | | NSLKTEDTAVYYCVRHGNFGNSYVSWFAYWGQGTLVTVS | |
| | | SGGGGSGGGGSGGGGSGGGGSQAVVTQEPSLTVSPGGT | |
| | | VTLTCRSSTGAVTTSNYANWVQQKPGQAPRGLIGGTNKRA | |
| | | PWTPARFSGSLLGDKAALTLSGAQPEDEAEYFCALWYSNL | |
| | | WVFGGGTKLTVLGGGGSDKTHTCPPCPAPELLGGPSVFLF | |
| | | PPKPKDTLMISRTPEVTCVVVAVSHEDPEVKFNWYVDGVE | |
| | | VHNAKTKPREEQYASTYRVVSVLTVLHQDWLNGKEYKCKV | |
| | | SNKALAAPIEKTISKAKGQPREPQVCTLPPSREEMTKNQVS | |
| | | LSCAVKGFYPSDIAVEWESNGQPENNYKTTPPVLDSDGSF | |
| | | FLVSKLTVDKSRWQQGNVFSCSVMHEALHNHYTQKSLSLS | |
| | | PGK | |
| | First Half | EIVMTQSPATLSVSPGERATLSCRASQDVGTAVAWYQQKP | 1431 |
| | Antibody Light | GQAPRLLIYWASTRHTGIPARFSGSGSGTEFTLTISSLQSE | |
| | Chain | DFAVYFCQQYANFPLYTFGQGTKLEIKRTVAAPSVFIFPPS | |
| | | DEQLKSGTASVVCLLNNFYPREAKVQWKVDNALQSGNSQ | |
| | | ESVTEQDSKDSTYSLSSTLTLSKADYEKHKVYACEVTHQGL | |
| | | SSPVTKSFNRGEC | |
| | Second Half | DKTHTCPPCPAPELLGGPSVFLFPPKPKDTLMISRTPEVTC | 1432 |
| | Antibody | VVVAVSHEDPEVKFNWYVDGVEVHNAKTKPREEQYASTY | |
| | | RVVSVLTVLHQDWLNGKEYKCKVSNKALAAPIEKTISKAKG | |
| | | QPREPQVYTLPPCREEMTKNQVSLWCLVKGFYPSDIAVEW | |
| | | ESNGQPENNYKTTPPVLDSDGSFFLYSKLTVDKSRWQQG | |
| | | NVFSCSVMHEALHNRYTQKSLSLSPGK | |

### 8.6.2. Results

As shown in FIG. 4A, addition of 20 nM CD2×CD20 BBM augmented NFAT reporter activity induced by the CD3×CD19 BBM. NFAT reporter activity was enhanced by the CD2×CD20 BBM in a dose-dependent manner in the presence of CD3×CD19 BBM stimulation (0.8 nM), while CD2×CD20 BBM had no effects on NFAT reporter activity in the absence of CD3×CD19 BBM (FIG. 4B). Thus, the results show that the CD2×CD20 BBM enhances CD3×CD19 BBM-induced T cell activation.

### 8.7. Example 7: CD2×CD20 BBM in Combination with CD3×CD19 BBM Induces Potent Tumor Cell Killing by Primary Human T Cells

The effect of a CD2×CD20 BBM on CD3×CD19 BBM-induced tumor cell killing assay was evaluated. A CD19×CD3×CD2 TBM was used for comparison.

### 8.7.1. Materials and Methods

The amino acid sequences of the CD2×CD20 BBM and CD3×CD19 BBM are shown in Tables 30A-30B (Example 6), and the CD3×CD19×CD2 TBM sequences are shown in Table 31. The TBM comprised a first half antibody comprising, in the N-terminal to C-terminal direction an anti-CD19 Fab, an anti-CD3 scFv, and first Fc region, and a second half antibody comprising the IgV domain of CD58 N-terminal to a second Fc region. The Fc regions of the TBM included knob-into-hole mutations to facilitate heterodimerization as well as silencing mutations. The CD2×CD20 BBM, CD3×CD19 BBM and CD3×CD19×CD2 TBM were expressed in mammalian cells and purified before setting up a tumor cell killing assay. Briefly, Karpas422 cells were engineered to constitutively express a firefly luciferase gene. Cells were harvested and resuspended in RPMI medium (Invitrogen #11875-093) with 10% FBS. 5,000 Karpas422 tumor cells were added in each well of a flat-bottom 384-well plate. Human pan T cells were isolated via negative selection (Miltenyi 130-096-535) from cryopreserved PBMCs (two donor) that were separated from a leukopak (Hemacare #PB001F-1) by Ficoll density gradient centrifugation. Purified T cells were then added to the plate to obtain a final effector cell:target cell (E:T) ratio of 1:1. Co-cultured cells were incubated with serial dilutions of CD2×CD20 BBM (in the absence or presence of 0.032 nM CD3×CD19 BBM), CD3×CD19 BBM (in the absence or presence of 20 nM CD2×CD20 BBM) or CD3×CD19×CD2 TBM. After an incubation of 96 hr at 37° C., 5% CO2, Bright-Glo luciferase substrate (Promega #E2650) was added to the plate. Luminescence was measured on an Envision plate reader after a 5 minute incubation. Percent specific lysis was calculated using the following equation: Specific lysis (%)=(1−(sample luminescence/average maximum luminescence))*100

TABLE 31
Amino acid sequences

| Construct | Chain | | SEQ |
|---|---|---|---|
| Name | Description | Amino Acid Sequence | ID NO: |
| CD3xCD19 | First Half | QVQLVQSGAEVKKPGASVKVSCKASGYTFTTYWIQWVRQ | 1430 |
| xCD2 TBM | Antibody | APGQRLEWMGAVYPGDADTRYTQKFQGRVTLTADRSAST | |
| | Heavy Chain | AYMELSSLRSEDTAVYYCGRDAGLEYYALDYWGQGTLVTV | |
| | | SSASTKGPSVFPLAPSSKSTSGGTAALGCLVKDYFPEPVTV | |
| | | SWNSGALTSGVHTFPAVLQSSGLYSLSSVVTVPSSSLGTQ | |
| | | TYICNVNHKPSNTKVDKRVEPKSCGGGGSGGGGSEVQLV | |
| | | ESGGGLVQPGGSLKLSCAASGFTFNTYAMNWVRQASGKG | |
| | | LEWVGRIRSKYNNYATYYADSVKDRFTISRDDSKSTLYLQM | |
| | | NSLKTEDTAVYYCVRHGNFGNSYVSWFAYWGQGTLVTVS | |
| | | SGGGGSGGGGSGGGGSGGGGSQAVVTQEPSLTVSPGGT | |
| | | VTLTCRSSTGAVTTSNYANWVQQKPGQAPRGLIGGTNKRA | |
| | | PWTPARFSGSLLGDKAALTLSGAQPEDEAEYFCALWYSNL | |
| | | WVFGGGTKLTVLGGGGSDKTHTCPPCPAPELLGGPSVFLF | |
| | | PPKPKDTLMISRTPEVTCVVVAVSHEDPEVKFNWYVDGVE | |
| | | VHNAKTKPREEQYASTYRVVSVLTVLHQDWLNGKEYKCKV | |
| | | SNKALAAPIEKTISKAKGQPREPQVCTLPPSREEMTKNQVS | |
| | | LSCAVKGFYPSDIAVEWESNGQPENNYKTTPPVLDSDGSF | |
| | | FLVSKLTVDKSRWQQGNVFSCSVMHEALHNHYTQKSLSLS | |
| | | PGK | |
| | First Half | EIVMTQSPATLSVSPGERATLSCRASQDVGTAVAWYQQKP | 1431 |
| | Antibody Light | GQAPRLLIYWASTRHTGIPARFSGSGSGTEFTLTISSLQSE | |
| | Chain | DFAVYFCQQYANFPLYTFGQGTKLEIKRTVAAPSVFIFPPS | |
| | | DEQLKSGTASVVCLLNNFYPREAKVQWKVDNALQSGNSQ | |
| | | ESVTEQDSKDSTYSLSSTLTLSKADYEKHKVYACEVTHQGL | |
| | | SSPVTKSFNRGEC | |
| | Second Half | SQQIYGVVYGNVTFHVPSNVPLKEVLWKKQKDKVAELENS | 1475 |
| | Antibody | EFRAFSSFKNRVYLDTVSGSLTIYNLTSSDEDEYEMESPNIT | |
| | | DTMKFFLYVLESGGGGSDKTHTCPPCPAPELLGGPSVFLF | |
| | | PPKPKDTLMISRTPEVTCVVVAVSHEDPEVKFNWYVDGVE | |
| | | VHNAKTKPREEQYASTYRVVSVLTVLHQDWLNGKEYKCKV | |
| | | SNKALAAPIEKTISKAKGQPREPQVYTLPPCREEMTKNQVS | |
| | | LWCLVKGFYPSDIAVEWESNGQPENNYKTTPPVLDSDGSF | |
| | | FLYSKLTVDKSRWQQGNVFSCSVMHEALHNRYTQKSLSLS | |
| | | PGK | |

### 8.7.2. Results

As shown in FIG. 5A, addition of 20 nM CD2×CD20 BBM enhanced CD3×CD19 BBM-induced tumor cell killing to a similar level as CD3×CD19×CD2 TBM. CD2×CD20 BBM augmented CD3×CD19 BBM-induced tumor cell killing in a dose-dependent manner in the presence of suboptimal CD3×CD19 BBM dose (0.032 nM), while CD2×CD20 BBM had no effects on tumor cell survival in the absence of CD3×CD19 BBM (FIG. 5B).

### 9. SPECIFIC EMBODIMENTS

While various specific embodiments have been illustrated and described, it will be appreciated that various changes can be made without departing from the spirit and scope of the disclosure(s). The present disclosure is exemplified by the numbered embodiments set forth below.

1. A method of treating a subject having a proliferative disease or an autoimmune disorder, comprising administering to the subject:
(a) a first multispecific binding molecule (“a first MBM”) comprising (i) an antigen-binding module 1 (ABM1) that binds specifically to human CD2 and (ii) an antigen-binding module 2 (ABM2) that binds specifically to a human tumor-associated antigen (“TAA”) and/or an antigen-binding module 3 (ABM3) that binds specifically to a human tumor microenvironment antigen (“TMEA”); and
(b) a second multispecific binding molecule (“a second MBM”) comprising (i) an antigen-binding module 4 (ABM4) that binds specifically to a component of a human T-cell receptor (TCR) complex or a secondary T-cell signaling molecule and (ii) an antigen-binding module 5 (ABM5) that binds specifically to a human tumor-associated antigen and/or an antigen-binding module 6 (ABM6) that binds specifically to a human tumor microenvironment antigen.

2. A combination of multispecific binding molecules for treating a subject having a proliferative disease or an autoimmune disorder, the combination comprising:
(a) a first multispecific binding molecule (“a first MBM”) comprising (i) an antigen-binding module 1 (ABM1) that binds specifically to human CD2 and (ii) an antigen-binding module 2 (ABM2) that binds specifically to a human tumor-associated antigen and/or an antigen-binding module 3 (ABM3) that binds specifically to a human tumor microenvironment antigen; and
(b) a second multispecific binding molecule (“a second MBM”) comprising (i) an antigen-binding module 4 (ABM4) that binds specifically to a component of a human T-cell receptor (TCR) complex or a secondary T-cell signaling molecule and (ii) an antigen-binding module 5 (ABM5) that binds specifically to a human tumor-associated antigen and/or an antigen-binding module 6 (ABM6) that binds specifically to a human tumor microenvironment antigen.

3. The method of embodiment 1 or combination of embodiment 2, wherein ABM1 is a non-immunoglobulin scaffold based ABM.

4. The method or combination of embodiment 3, wherein ABM1 is a Kunitz domain, an Adnexin, an Affibody, a DARPin, an Avimer, an Anticalin, a Lipocalin, a Centyrin, a Versabody, a Knottin, an Adnectin, a Pronectin, an Affitin/Nanofitin, an Affilin, an Atrimer/Tetranectin, a bicyclic peptide, a cys-knot, a Fn3 scaffold, an Obody, a Tn3, an Affimer, BD, an Adhiron, a Duocalin, an Alphabody, an Armadillo Repeat Protein, a Repebody, or a Fynomer.

5. The method or combination of any one of embodiments 1 to 4, wherein ABM1 comprises a receptor binding domain of a CD2 ligand.

6. The method or combination of embodiment 5, wherein the CD2 ligand is CD58.

7. The method or combination of embodiment 5, wherein the CD2 ligand is CD48.

8. The method or combination of any one of embodiments 1 to 6, wherein ABM1 is a CD58 moiety.

9. The method or combination of embodiment 8, wherein the CD58 moiety comprises the amino acid sequence of CD58-1 as set forth in Table 12.

10. The method or combination of embodiment 8, wherein the CD58 moiety comprises the amino acid sequence of CD58-2 as set forth in Table 12.

11. The method or combination of embodiment 8, wherein the CD58 moiety comprises the amino acid sequence of CD58-3 as set forth in Table 12.

12. The method or combination of embodiment 8, wherein the CD58 moiety comprises the amino acid sequence of CD58-4 as set forth in Table 12.

13. The method or combination of embodiment 8, wherein the CD58 moiety comprises the amino acid sequence of CD58-5 as set forth in Table 12.

14. The method or combination of embodiment 13, wherein the amino acid designated as B is a phenylalanine.

15. The method or combination of embodiment 13, wherein the amino acid designated as B is a serine.

16. The method or combination of any one of embodiments 13 to 15, wherein the amino acid designated as J is a valine.

17. The method or combination of any one of embodiments 13 to 15, wherein the amino acid designated as J is a lysine.

18. The method or combination of any one of embodiments 13 to 17, wherein the amino acid designated as O is a valine.

19. The method or combination of any one of embodiments 13 to 17, wherein the amino acid designated as O is a glutamine.

20. The method or combination of any one of embodiments 13 to 19, wherein the amino acid designated as U is a valine.

21. The method or combination of any one of embodiments 13 to 19, wherein the amino acid designated as U is a lysine.

22. The method or combination of any one of embodiments 13 to 21, wherein the amino acid designated as X is a threonine.

23. The method or combination of any one of embodiments 13 to 21, wherein the amino acid designated as X is a serine.

24. The method or combination of any one of embodiments 13 to 23, wherein the amino acid designated as Z is a leucine.

25. The method or combination of any one of embodiments 13 to 23, wherein the amino acid designated as Z is a glycine.

26. The method or combination of embodiment 8, wherein the CD58 moiety comprises the amino acid sequence of CD58-6 as set forth in Table 12.

27. The method or combination of embodiment 8, wherein the CD58 moiety comprises the amino acid sequence of CD58-7 as set forth in Table 12.

28. The method or combination of embodiment 27, wherein the amino acid designated as J is a valine.

29. The method or combination of embodiment 27, wherein the amino acid designated as J is a lysine.

30. The method or combination of any one of embodiments 27 to 29, wherein the amino acid designated as O is a valine.

31. The method or combination of any one of embodiments 27 to 29, wherein the amino acid designated as O is a glutamine.

32. The method or combination of embodiment 8, wherein the CD58 moiety comprises the amino acid sequence of CD58-8 as set forth in Table 12.

33. The method or combination of embodiment 8, wherein the CD58 moiety comprises the amino acid sequence of CD58-9 as set forth in Table 12.

34. The method or combination of embodiment 8, wherein the CD58 moiety comprises the amino acid sequence of CD58-10 as set forth in Table 12.

35. The method or combination of embodiment 8, wherein the CD58 moiety comprises the amino acid sequence of CD58-11 as set forth in Table 12.

36. The method or combination of any one of embodiments 1 to 5, wherein ABM1 is a CD48 moiety.

37. The method or combination of embodiment 36, wherein the CD48 moiety has at least 70% sequence identity to amino acids 27-220 of the amino acid sequence of Uniprot identifier P09326.

38. The method or combination of embodiment 36, wherein the CD48 moiety has at least 80% sequence identity to amino acids 27-220 of the amino acid sequence of Uniprot identifier P09326.

39. The method or combination of embodiment 36, wherein the CD48 moiety has at least 90% sequence identity to amino acids 27-220 of the amino acid sequence of Uniprot identifier P09326.

40. The method or combination of embodiment 36, wherein the CD48 moiety has at least 95% sequence identity to f amino acids 27-220 of the amino acid sequence of Uniprot identifier P09326.

41. The method or combination of embodiment 36, wherein the CD48 moiety has at least 99% sequence identity to amino acids 27-220 of the amino acid sequence of Uniprot identifier P09326.

42. The method of embodiment 1 or combination of embodiment 2, wherein ABM1 is an immunoglobulin scaffold based ABM.

43. The method or combination of embodiment 42, wherein ABM1 is an antibody, an antibody fragment, an scFv, a dsFv, a Fv, a Fab, an scFab, a (Fab′)2, a single domain antibody (SDAB), a VH or VL domain, or a camelid VHH domain.

44. The method or combination of embodiment 43, wherein ABM1 is an antibody or an antigen-binding domain thereof.

45. The method or combination of embodiment 43, wherein ABM1 is an scFv.

46. The method or combination of embodiment 43, wherein ABM1 is a Fab.

47. The method or combination of embodiment 46, wherein ABM1 is a Fab heterodimer.

48. The method or combination of any one of embodiments 42 to 47, wherein ABM1 comprises the CDR sequences of CD2-1.

49. The method or combination of embodiment 48, wherein ABM1 comprises the heavy and light chain variable sequences of CD2-1.

50. The method or combination of embodiment 48, wherein ABM1 comprises the heavy and light chain variable sequences of hu1CD2-1.

51. The method or combination of embodiment 48, wherein ABM1 comprises the heavy and light chain variable sequences of hu2CD2-1.

52. The method or combination of any one of embodiments 42 to 47, wherein ABM1 comprises the CDR sequences of CD2_2 as defined by Kabat and set forth in Table 11B.

53. The method or combination of any one of embodiments 42 to 47, wherein ABM1 comprises the CDR sequences of CD2_2 as defined by Chothia and set forth in Table 11B.

54. The method or combination of any one of embodiments 42 to 47, wherein ABM1 comprises the CDR sequences of CD2_2 as defined by IMGT and set forth in Table 11B.

55. The method or combination of any one of embodiments 42 to 47, wherein ABM1 comprises the CDR sequences of CD2_2 as defined by the combination of Kabat and Chothia and set forth in Table 11B.

56. The method or combination of any one of embodiments 42 to 47, wherein ABM1 comprises the heavy chain and/or light chain variable sequences of CD2_2 as set forth in Table 11B.

57. The method or combination of any one of embodiments 42 to 47, wherein ABM1 comprises the CDR sequences of Medi 507.

58. The method or combination of embodiment 52, wherein ABM1 comprises the heavy and light chain variable sequences of Medi 507.

59. The method or combination of any one of embodiments 1 to 58, wherein ABM4 binds specifically to a component of a TCR complex.

60. The method or combination of embodiment 59, wherein ABM4 is a non-immunoglobulin scaffold based ABM.

61. The method or combination of embodiment 60, wherein ABM4 is a Kunitz domain, an Adnexin, an Affibody, a DARPin, an Avimer, an Anticalin, a Lipocalin, a Centyrin, a Versabody, a Knottin, an Adnectin, a Pronectin, an Affitin/Nanofitin, an Affilin, an Atrimer/Tetranectin, a bicyclic peptide, a cys-knot, a Fn3 scaffold, an Obody, a Tn3, an Affimer, BD, an Adhiron, a Duocalin, an Alphabody, an Armadillo Repeat Protein, a Repebody, or a Fynomer.

62. The method or combination of embodiment 59, wherein ABM4 is an immunoglobulin scaffold based ABM.

63. The method or combination of embodiment 62, wherein ABM4 is an antibody, an antibody fragment, an scFv, a dsFv, a Fv, a Fab, an scFab, a (Fab′)2, a single domain antibody (SDAB), a VH or VL domain, or a camelid VHH domain.

64. The method or combination of embodiment 63, wherein ABM4 is an antibody or an antigen-binding domain thereof.

65. The method or combination of embodiment 63, wherein ABM4 is an scFv.

66. The method or combination of embodiment 63, wherein ABM4 is a Fab.

67. The method or combination of embodiment 66, wherein ABM4 is a Fab heterodimer.

68. The method or combination of any one of embodiments 59 to 67, wherein the component of the TCR complex is CD3.

69. The method or combination of embodiment 68, wherein ABM4 comprises the CDR sequences of CD3-1.

70. T The method or combination of embodiment 68, wherein ABM4 comprises the CDR sequences of CD3-2.

71. The method or combination of embodiment 68, wherein ABM4 comprises the CDR sequences of CD3-3.

72. The method or combination of embodiment 68, wherein ABM4 comprises the CDR sequences of CD3-4.

73. The method or combination of embodiment 68, wherein ABM4 comprises the CDR sequences of CD3-5.

74. The method or combination of embodiment 68, wherein ABM4 comprises the CDR sequences of CD3-6.

75. The method or combination of embodiment 68, wherein ABM4 comprises the CDR sequences of CD3-7.

76. The method or combination of embodiment 68, wherein ABM4 comprises the CDR sequences of CD3-8.

77. The method or combination of embodiment 68, wherein ABM4 comprises the CDR sequences of CD3-9.

78. The method or combination of embodiment 68, wherein ABM4 comprises the CDR sequences of CD3-10.

79. The method or combination of embodiment 68, wherein ABM4 comprises the CDR sequences of CD3-11.

80. The method or combination of embodiment 68, wherein ABM4 comprises the CDR sequences of CD3-12.

81. The method or combination of embodiment 68, wherein ABM4 comprises the CDR sequences of CD3-13.

82. The method or combination of embodiment 68, wherein ABM4 comprises the CDR sequences of CD3-14.

83. The method or combination of embodiment 68, wherein ABM4 comprises the CDR sequences of CD3-15.

84. The method or combination of embodiment 68, wherein ABM4 comprises the CDR sequences of CD3-16.

85. The method or combination of embodiment 68, wherein ABM4 comprises the CDR sequences of CD3-17.

86. The method or combination of embodiment 68, wherein ABM4 comprises the CDR sequences of CD3-18.

87. The method or combination of embodiment 68, wherein ABM4 comprises the CDR sequences of CD3-19.

88. The method or combination of embodiment 68, wherein ABM4 comprises the CDR sequences of CD3-20.

89. The method or combination of embodiment 68, wherein ABM4 comprises the CDR sequences of CD3-21.

90. The method or combination of embodiment 68, wherein ABM4 comprises the CDR sequences of CD3-22.

91. The method or combination of embodiment 68, wherein ABM4 comprises the CDR sequences of CD3-23.

92. The method or combination of embodiment 68, wherein ABM4 comprises the CDR sequences of CD3-24.

93. The method or combination of embodiment 68, wherein ABM4 comprises the CDR sequences of CD3-25.

94. The method or combination of embodiment 68, wherein ABM4 comprises the CDR sequences of CD3-26.

95. The method or combination of embodiment 68, wherein ABM4 comprises the CDR sequences of CD3-27.

96. The method or combination of embodiment 68, wherein ABM4 comprises the CDR sequences of CD3-28.

97. The method or combination of embodiment 68, wherein ABM4 comprises the CDR sequences of CD3-29.

98. The method or combination of embodiment 68, wherein ABM4 comprises the CDR sequences of CD3-30.

99. The method or combination of embodiment 68, wherein ABM4 comprises the CDR sequences of CD3-31.

100. The method or combination of embodiment 68, wherein ABM4 comprises the CDR sequences of CD3-32.

101. The method or combination of embodiment 68, wherein ABM4 comprises the CDR sequences of CD3-33.

102. The method or combination of embodiment 68, wherein ABM4 comprises the CDR sequences of CD3-34.

103. The method or combination of embodiment 68, wherein ABM4 comprises the CDR sequences of CD3-35.

104. The method or combination of embodiment 68, wherein ABM4 comprises the CDR sequences of CD3-36.

105. The method or combination of embodiment 68, wherein ABM4 comprises the CDR sequences of CD3-37.

106. The method or combination of embodiment 68, wherein ABM4 comprises the CDR sequences of CD3-38.

107. The method or combination of embodiment 68, wherein ABM4 comprises the CDR sequences of CD3-39.

108. The method or combination of embodiment 68, wherein ABM4 comprises the CDR sequences of CD3-40.

109. The method or combination of embodiment 68, wherein ABM4 comprises the CDR sequences of CD3-41.

110. The method or combination of embodiment 68, wherein ABM4 comprises the CDR sequences of CD3-42.

111. The method or combination of embodiment 68, wherein ABM4 comprises the CDR sequences of CD3-43.

112. The method or combination of embodiment 68, wherein ABM4 comprises the CDR sequences of CD3-44.

113. The method or combination of embodiment 68, wherein ABM4 comprises the CDR sequences of CD3-45.

114. The method or combination of embodiment 68, wherein ABM4 comprises the CDR sequences of CD3-46.

115. The method or combination of embodiment 68, wherein ABM4 comprises the CDR sequences of CD3-47.

116. The method or combination of embodiment 68, wherein ABM4 comprises the CDR sequences of CD3-48.

117. The method or combination of embodiment 68, wherein ABM4 comprises the CDR sequences of CD3-49.

118. The method or combination of embodiment 68, wherein ABM4 comprises the CDR sequences of CD3-50.

119. The method or combination of embodiment 68, wherein ABM4 comprises the CDR sequences of CD3-51.

120. The method or combination of embodiment 68, wherein ABM4 comprises the CDR sequences of CD3-52.

121. The method or combination of embodiment 68, wherein ABM4 comprises the CDR sequences of CD3-53.

122. The method or combination of embodiment 68, wherein ABM4 comprises the CDR sequences of CD3-54.

123. The method or combination of embodiment 68, wherein ABM4 comprises the CDR sequences of CD3-55.

124. The method or combination of embodiment 68, wherein ABM4 comprises the CDR sequences of CD3-56.

125. The method or combination of embodiment 68, wherein ABM4 comprises the CDR sequences of CD3-57.

126. The method or combination of embodiment 68, wherein ABM4 comprises the CDR sequences of CD3-58.

127. The method or combination of embodiment 68, wherein ABM4 comprises the CDR sequences of CD3-59.

128. The method or combination of embodiment 68, wherein ABM4 comprises the CDR sequences of CD3-60.

129. The method or combination of embodiment 68, wherein ABM4 comprises the CDR sequences of CD3-61.

130. The method or combination of embodiment 68, wherein ABM4 comprises the CDR sequences of CD3-62.

131. The method or combination of embodiment 68, wherein ABM4 comprises the CDR sequences of CD3-63.

132. The method or combination of embodiment 68, wherein ABM4 comprises the CDR sequences of CD3-64.

133. The method or combination of embodiment 68, wherein ABM4 comprises the CDR sequences of CD3-65.

134. The method or combination of embodiment 68, wherein ABM4 comprises the CDR sequences of CD3-66.

135. The method or combination of embodiment 68, wherein ABM4 comprises the CDR sequences of CD3-67.

136. The method or combination of embodiment 68, wherein ABM4 comprises the CDR sequences of CD3-68.

137. The method or combination of embodiment 68, wherein ABM4 comprises the CDR sequences of CD3-69.

138. The method or combination of embodiment 68, wherein ABM4 comprises the CDR sequences of CD3-70.

139. The method or combination of embodiment 68, wherein ABM4 comprises the CDR sequences of CD3-71.

140. The method or combination of embodiment 68, wherein ABM4 comprises the CDR sequences of CD3-72.

141. The method or combination of embodiment 68, wherein ABM4 comprises the CDR sequences of CD3-73.

142. The method or combination of embodiment 68, wherein ABM4 comprises the CDR sequences of CD3-74.

143. The method or combination of embodiment 68, wherein ABM4 comprises the CDR sequences of CD3-75.

144. The method or combination of embodiment 68, wherein ABM4 comprises the CDR sequences of CD3-76.

145. The method or combination of embodiment 68, wherein ABM4 comprises the CDR sequences of CD3-77.

146. The method or combination of embodiment 68, wherein ABM4 comprises the CDR sequences of CD3-78.

147. The method or combination of embodiment 68, wherein ABM4 comprises the CDR sequences of CD3-79.

148. The method or combination of embodiment 68, wherein ABM4 comprises the CDR sequences of CD3-80.

149. The method or combination of embodiment 68, wherein ABM4 comprises the CDR sequences of CD3-81.

150. The method or combination of embodiment 68, wherein ABM4 comprises the CDR sequences of CD3-82.

151. The method or combination of embodiment 68, wherein ABM4 comprises the CDR sequences of CD3-83.

152. The method or combination of embodiment 68, wherein ABM4 comprises the CDR sequences of CD3-84.

153. The method or combination of embodiment 68, wherein ABM4 comprises the CDR sequences of CD3-85.

154. The method or combination of embodiment 68, wherein ABM4 comprises the CDR sequences of CD3-86.

155. The method or combination of embodiment 68, wherein ABM4 comprises the CDR sequences of CD3-87.

156. The method or combination of embodiment 68, wherein ABM4 comprises the CDR sequences of CD3-88.

157. The method or combination of embodiment 68, wherein ABM4 comprises the CDR sequences of CD3-89.

158. The method or combination of embodiment 68, wherein ABM4 comprises the CDR sequences of CD3-90.

159. The method or combination of embodiment 68, wherein ABM4 comprises the CDR sequences of CD3-91.

160. The method or combination of embodiment 68, wherein ABM4 comprises the CDR sequences of CD3-92.

161. The method or combination of embodiment 68, wherein ABM4 comprises the CDR sequences of CD3-93.

162. The method or combination of embodiment 68, wherein ABM4 comprises the CDR sequences of CD3-94.

163. The method or combination of embodiment 68, wherein ABM4 comprises the CDR sequences of CD3-95.

164. The method or combination of embodiment 68, wherein ABM4 comprises the CDR sequences of CD3-96.

165. The method or combination of embodiment 68, wherein ABM4 comprises the CDR sequences of CD3-97.

166. The method or combination of embodiment 68, wherein ABM4 comprises the CDR sequences of CD3-98.

167. The method or combination of embodiment 68, wherein ABM4 comprises the CDR sequences of CD3-99.

168. The method or combination of embodiment 68, wherein ABM4 comprises the CDR sequences of CD3-100.

169. The method or combination of embodiment 68, wherein ABM4 comprises the CDR sequences of CD3-101.

170. The method or combination of embodiment 68, wherein ABM4 comprises the CDR sequences of CD3-102.

171. The method or combination of embodiment 68, wherein ABM4 comprises the CDR sequences of CD3-103.

172. The method or combination of embodiment 68, wherein ABM4 comprises the CDR sequences of CD3-104.

173. The method or combination of embodiment 68, wherein ABM4 comprises the CDR sequences of CD3-105.

174. The method or combination of embodiment 68, wherein ABM4 comprises the CDR sequences of CD3-106.

175. The method or combination of embodiment 68, wherein ABM4 comprises the CDR sequences of CD3-107.

176. The method or combination of embodiment 68, wherein ABM4 comprises the CDR sequences of CD3-108.

177. The method or combination of embodiment 68, wherein ABM4 comprises the CDR sequences of CD3-109.

178. The method or combination of embodiment 68, wherein ABM4 comprises the CDR sequences of CD3-110.

179. The method or combination of embodiment 68, wherein ABM4 comprises the CDR sequences of CD3-111.

180. The method or combination of embodiment 68, wherein ABM4 comprises the CDR sequences of CD3-112.

181. The method or combination of embodiment 68, wherein ABM4 comprises the CDR sequences of CD3-113.

182. The method or combination of embodiment 68, wherein ABM4 comprises the CDR sequences of CD3-114.

183. The method or combination of embodiment 68, wherein ABM4 comprises the CDR sequences of CD3-115.

184. The method or combination of embodiment 68, wherein ABM4 comprises the CDR sequences of CD3-116.

185. The method or combination of embodiment 68, wherein ABM4 comprises the CDR sequences of CD3-117.

186. The method or combination of embodiment 68, wherein ABM4 comprises the CDR sequences of CD3-118.

187. The method or combination of embodiment 68, wherein ABM4 comprises the CDR sequences of CD3-119.

188. The method or combination of embodiment 68, wherein ABM4 comprises the CDR sequences of CD3-120.

189. The method or combination of embodiment 68, wherein ABM4 comprises the CDR sequences of CD3-121.

190. The method or combination of embodiment 68, wherein ABM4 comprises the CDR sequences of CD3-122.

191. The method or combination of embodiment 68, wherein ABM4 comprises the CDR sequences of CD3-123.

192. The method or combination of embodiment 68, wherein ABM4 comprises the CDR sequences of CD3-124.

193. The method or combination of embodiment 68, wherein ABM4 comprises the CDR sequences of CD3-125.

194. The method or combination of embodiment 68, wherein ABM4 comprises the CDR sequences of CD3-126.

195. The method or combination of embodiment 68, wherein ABM4 comprises the CDR sequences of CD3-127.

196. The method or combination of embodiment 68, wherein ABM4 comprises the CDR sequences of CD3-128.

197. The method or combination of embodiment 68, wherein ABM4 comprises the CDR sequences of CD3-129.

198. The method or combination of embodiment 68, wherein ABM4 comprises the CDR sequences of CD3-130.

199. The method or combination of any one of embodiments 69 to 198, wherein the CDRs are defined by Kabat numbering, as set forth in Table 22B.

200. The method or combination of any one of embodiments 69 to 198, wherein the CDRs are defined by Chothia numbering, as set forth in Table 22C.

201. The method or combination of any one of embodiments 69 to 198, wherein the CDRs are defined by a combination of Kabat and Chothia numbering, as set forth in Table 22D.

202. The method or combination of embodiment 68, wherein ABM4 comprises the heavy and light chain variable sequences of CD3-1, as set forth in Table 22A.

203. The method or combination of embodiment 68, wherein ABM4 comprises the heavy and light chain variable sequences of CD3-2, as set forth in Table 22A.

204. The method or combination of embodiment 68, wherein ABM4 comprises the heavy and light chain variable sequences of CD3-3, as set forth in Table 22A.

205. The method or combination of embodiment 68, wherein ABM4 comprises the heavy and light chain variable sequences of CD3-4, as set forth in Table 22A.

206. The method or combination of embodiment 68, wherein ABM4 comprises the heavy and light chain variable sequences of CD3-5, as set forth in Table 22A.

207. The method or combination of embodiment 68, wherein ABM4 comprises the heavy and light chain variable sequences of CD3-6, as set forth in Table 22A.

208. The method or combination of embodiment 68, wherein ABM4 comprises the heavy and light chain variable sequences of CD3-7, as set forth in Table 22A.

209. The method or combination of embodiment 68, wherein ABM4 comprises the heavy and light chain variable sequences of CD3-8, as set forth in Table 22A.

210. The method or combination of embodiment 68, wherein ABM4 comprises the heavy and light chain variable sequences of CD3-9, as set forth in Table 22A.

211. The method or combination of embodiment 68, wherein ABM4 comprises the heavy and light chain variable sequences of CD3-10, as set forth in Table 22A.

212. The method or combination of embodiment 68, wherein ABM4 comprises the heavy and light chain variable sequences of CD3-11, as set forth in Table 22A.

213. The method or combination of embodiment 68, wherein ABM4 comprises the heavy and light chain variable sequences of CD3-12, as set forth in Table 22A.

214. The method or combination of embodiment 68, wherein ABM4 comprises the heavy and light chain variable sequences of CD3-13, as set forth in Table 22A.

215. The method or combination of embodiment 68, wherein ABM4 comprises the heavy and light chain variable sequences of CD3-14, as set forth in Table 22A.

216. The method or combination of embodiment 68, wherein ABM4 comprises the heavy and light chain variable sequences of CD3-15, as set forth in Table 22A.

217. The method or combination of embodiment 68, wherein ABM4 comprises the heavy and light chain variable sequences of CD3-16, as set forth in Table 22A.

218. The method or combination of embodiment 68, wherein ABM4 comprises the heavy and light chain variable sequences of CD3-17, as set forth in Table 22A.

219. The method or combination of embodiment 68, wherein ABM4 comprises the heavy and light chain variable sequences of CD3-18, as set forth in Table 22A.

220. The method or combination of embodiment 68, wherein ABM4 comprises the heavy and light chain variable sequences of CD3-19, as set forth in Table 22A.

221. The method or combination of embodiment 68, wherein ABM4 comprises the heavy and light chain variable sequences of CD3-20, as set forth in Table 22A.

222. The method or combination of embodiment 68, wherein ABM4 comprises the heavy and light chain variable sequences of CD3-21, as set forth in Table 22A.

223. The method or combination of embodiment 68, wherein ABM4 comprises the heavy and light chain variable sequences of CD3-22, as set forth in Table 22A.

224. The method or combination of embodiment 68, wherein ABM4 comprises the heavy and light chain variable sequences of CD3-23, as set forth in Table 22A.

225. The method or combination of embodiment 68, wherein ABM4 comprises the heavy and light chain variable sequences of CD3-24, as set forth in Table 22A.

226. The method or combination of embodiment 68, wherein ABM4 comprises the heavy and light chain variable sequences of CD3-25, as set forth in Table 22A.

227. The method or combination of embodiment 68, wherein ABM4 comprises the heavy and light chain variable sequences of CD3-26, as set forth in Table 22A.

228. The method or combination of embodiment 68, wherein ABM4 comprises the heavy and light chain variable sequences of CD3-27, as set forth in Table 22A.

229. The method or combination of embodiment 68, wherein ABM4 comprises the heavy and light chain variable sequences of CD3-28, as set forth in Table 22A.

230. The method or combination of embodiment 68, wherein ABM4 comprises the heavy and light chain variable sequences of CD3-129, as set forth in Table 22A.

231. The method or combination of embodiment 68, wherein ABM4 comprises the heavy and light chain variable sequences of CD3-130, as set forth in Table 22A.

232. The method or combination of embodiment 68, wherein ABM4 comprises the amino acid sequence of the scFv designated as CD3-12 in Table 22A.

233. The method or combination of embodiment 68, wherein ABM4 comprises the amino acid sequence of the scFv designated as CD3-21 in Table 22A.

234. The method or combination of embodiment 68, wherein ABM4 comprises the amino acid sequence of the scFv designated as CD3-22 in Table 22A.

235. The method or combination of embodiment 68, wherein ABM4 comprises the amino acid sequence of the scFv designated as CD3-23 in Table 22A.

236. The method or combination of embodiment 68, wherein ABM4 comprises the amino acid sequence of the scFv designated as CD3-24 in Table 22A.

237. The method or combination of embodiment 68, wherein ABM4 comprises the amino acid sequence of the scFv designated as CD3-25 in Table 22A.

238. The method or combination of embodiment 68, wherein ABM4 comprises the amino acid sequence of the scFv designated as CD3-26 in Table 22A.

239. The method or combination of embodiment 68, wherein ABM4 comprises the amino acid sequence of the scFv designated as CD3-27 in Table 22A.

240. The method or combination of embodiment 68, wherein ABM4 comprises the amino acid sequence of the scFv designated as CD3-28 in Table 22A.

241. The method or combination of embodiment 68, wherein ABM4 comprises the amino acid sequence of the scFv designated as CD3-129 in Table 22A.

242. The method or combination of embodiment 68, wherein ABM4 comprises the amino acid sequence of the scFv designated as CD3-130 in Table 22A.

243. The method or combination of embodiment 68, wherein ABM4 comprises CDR-H1, CDR-H2, CDR-H3, CDR-L1, CDR-L2, and CDR-L3 sequences selected from the CDR-H1, CDR-H2, CDR-H3, CDR-L1, CDR-L2, and CDR-L3 sequences set forth in Table 1A of WO 2020/052692.

244. The method or combination of embodiment 68, wherein ABM4 comprises CDR-H1, CDR-H2, CDR-H3, CDR-L1, CDR-L2, and CDR-L3 sequences selected from the CDR-H1, CDR-H2, CDR-H3, CDR-L1, CDR-L2, and CDR-L3 sequences set forth in Table 1B of WO 2020/052692.

245. The method or combination of embodiment 68, wherein ABM4 comprises comprises CDR-H1, CDR-H2, CDR-H3, CDR-L1, CDR-L2, and CDR-L3 sequences selected from the CDR-H1, CDR-H2, CDR-H3, CDR-L1, CDR-L2, and CDR-L3 sequences set forth in Table 10 of WO 2020/052692.

246. The method or combination of embodiment 68, wherein ABM4 comprises the CDR-H1, CDR-H2, and CDR-H3 sequences of one of the CD3 binders set forth in Table 1D-1 of WO 2020/052692 and the corresponding CDR-L1, CDR-L2, and CDR-L3 sequences set forth in 1D-2 of WO 2020/052692.

247. The method or combination of embodiment 68, wherein ABM4 comprises the CDR-H1, CDR-H2, and CDR-H3 sequences of one of the CD3 binders set forth in Table 1E-1 of WO 2020/052692 and the corresponding CDR-L1, CDR-L2, and CDR-L3 sequences set forth in 1E-2 of WO 2020/052692.

248. The method or combination of embodiment 68, wherein ABM4 comprises the CDR-H1, CDR-H2, and CDR-H3 sequences of one of the CD3 binders set forth in Table 1F-1 of WO 2020/052692 and the corresponding CDR-L1, CDR-L2, and CDR-L3 sequences set forth in 1F-2 of WO 2020/052692.

249. The method or combination of embodiment 68, wherein ABM4 comprises the CDR-H1, CDR-H2, and CDR-H3 sequences of one of the CD3 binders set forth in Table 1G-1 of WO 2020/052692 and the corresponding CDR-L1, CDR-L2, and CDR-L3 sequences set forth in 1G-2 of WO 2020/052692.

250. The method or combination of embodiment 68, wherein ABM4 comprises the CDR-H1, CDR-H2, and CDR-H3 sequences of one of the CD3 binders set forth in Table 1H-1 of WO 2020/052692 and the corresponding CDR-L1, CDR-L2, and CDR-L3 sequences set forth in 1H-2 of WO 2020/052692.

251. The method or combination of embodiment 68, wherein ABM4 comprises the CDR-H1, CDR-H2, and CDR-H3 sequences of one of the CD3 binders set forth in Table 11-1 of WO 2020/052692 and the corresponding CDR-L1, CDR-L2, and CDR-L3 sequences set forth in 11-2 of WO 2020/052692.

252. The method or combination of embodiment 68, wherein ABM4 comprises a VH sequence of one of the CD3 binders set forth in Table 1J-1 2 of WO 2020/052692 and the the corresponding VL sequence set forth in Table 1J-2 of WO 2020/052692.

253. The method or combination of any one of embodiments 59 to 67, wherein the component of the TCR complex is TCR-α, TCR-β, or a TCR-α/β dimer.

254. The method or combination of embodiment 253, wherein the component of the TCR complex is TCR-α.

255. The method or combination of embodiment 253, wherein the component of the TCR complex is TCR-β.

256. The method or combination of embodiment 253, wherein the component of the TCR complex is a TCR-α/β dimer.

257. The method or combination of embodiment 253, wherein ABM4 comprises the CDR sequences of BMA031.

258. The method or combination of embodiment 257, wherein the CDR sequences are defined by Kabat numbering.

259. The method or combination of embodiment 257, wherein the CDR sequences are defined by Chothia numbering.

260. The method or combination of embodiment 257, wherein the CDR sequences are defined by a combination of Kabat and Chothia numbering.

261. The method or combination of embodiment 257, wherein ABM4 comprises the heavy and light chain variable sequences of BMA031.

262. The method or combination of any one of embodiments 59 to 67, wherein the component of the TCR complex is TCR-γ, TCR-δ, or a TCR-γ/δ dimer.

263. The method or combination of embodiment 262, wherein the component of the TCR complex is TCR-γ.

264. The method or combination of embodiment 262, wherein the component of the TCR complex is TCR-δ.

265. The method or combination of embodiment 262, wherein the component of the TCR complex is a TCR-γ/δ dimer.

266. The method or combination of embodiment 262, wherein ABM4 comprises the CDR sequences of δTCS1.

267. The method or combination of embodiment 266, wherein the CDR sequences are defined by Kabat numbering.

268. The method or combination of embodiment 266, wherein the CDR sequences are defined by Chothia numbering.

269. The method or combination of embodiment 266, wherein the CDR sequences are defined by a combination of Kabat and Chothia numbering.

270. The method or combination of embodiment 266, wherein ABM4 comprises the heavy and light chain variable sequences of δTCS1.

271. The method or combination of any one of embodiments 1 to 58, wherein ABM4 binds specifically to a secondary T-cell signaling molecule.

272. The method or combination of embodiment 271, wherein ABM4 is a non-immunoglobulin scaffold based ABM.

273. The method or combination of embodiment 272, wherein ABM4 is a Kunitz domain, an Adnexin, an Affibody, a DARPin, an Avimer, an Anticalin, a Lipocalin, a Centyrin, a Versabody, a Knottin, an Adnectin, a Pronectin, an Affitin/Nanofitin, an Affilin, an Atrimer/Tetranectin, a bicyclic peptide, a cys-knot, a Fn3 scaffold, an Obody, a Tn3, an Affimer, BD, an Adhiron, a Duocalin, an Alphabody, an Armadillo Repeat Protein, a Repebody, or a Fynomer.

274. The method or combination of embodiment 271, wherein ABM4 is an immunoglobulin scaffold based ABM.

275. The method or combination of embodiment 274, wherein ABM4 is an antibody, an antibody fragment, an scFv, a dsFv, a Fv, a Fab, an scFab, a (Fab′)2, a single domain antibody (SDAB), a VH or VL domain, or a camelid VHH domain.

276. The method or combination of embodiment 275, wherein ABM4 is an antibody or an antigen-binding domain thereof.

277. The method or combination of embodiment 275, wherein ABM4 is an scFv. 278. The method or combination of embodiment 275, wherein ABM4 is a Fab. 279. The method or combination of embodiment 278, wherein ABM4 is a Fab heterodimer.

280. The method or combination of any one of embodiments 271 to 279, wherein the secondary T-cell signaling molecule is a receptor.

281. The method or combination of any one of embodiments 271 to 279, wherein the secondary T-cell signaling molecule is a ligand.

282. The method or combination of any one of embodiments 271 to 279, wherein the secondary T-cell signaling molecule is CD27, CD28, CD30, CD40L, CD150, CD160, CD226, CD244, BTLA, BTN3A1, B7-1, CTLA4, DR3, GITR, HVEM, ICOS, LAG3, LAIR1, LIGHT, OX40, PD1, PDL1, PDL2, TIGIT, TIM1, TIM2, TIM3, VISTA, CD70, or 4-1BB.

283. The method or combination of embodiment 282, wherein the secondary T-cell signaling molecule is CD27.

284. The method or combination of embodiment 282, wherein the secondary T-cell signaling molecule is CD28.

285. The method or combination of embodiment 282, wherein the secondary T-cell signaling molecule is CD30.

286. The method or combination of embodiment 282, wherein the secondary T-cell signaling molecule is CD40L.

287. The method or combination of embodiment 282, wherein the secondary T-cell signaling molecule is CD150.

288. The method or combination of embodiment 282, wherein the secondary T-cell signaling molecule is CD160.

289. The method or combination of embodiment 282, wherein the secondary T-cell signaling molecule is CD226.

290. The method or combination of embodiment 282, wherein the secondary T-cell signaling molecule is CD244.

291. The method or combination of embodiment 282, wherein the secondary T-cell signaling molecule is BTLA.

292. The method or combination of embodiment 282, wherein the secondary T-cell signaling molecule is BTN3A1.

293. The method or combination of embodiment 282, wherein the secondary T-cell signaling molecule is B7-1.

294. The method or combination of embodiment 282, wherein the secondary T-cell signaling molecule is CTLA4.

295. The method or combination of embodiment 282, wherein the secondary T-cell signaling molecule is DR3.

296. The method or combination of embodiment 282, wherein the secondary T-cell signaling molecule is GITR.

297. The method or combination of embodiment 282, wherein the secondary T-cell signaling molecule is HVEM.

298. The method or combination of embodiment 282, wherein the secondary T-cell signaling molecule is ICOS.

299. The method or combination of embodiment 282, wherein the secondary T-cell signaling molecule is LAG3.

300. The method or combination of embodiment 282, wherein the secondary T-cell signaling molecule is LAIR1.

301. The method or combination of embodiment 282, wherein the secondary T-cell signaling molecule is LIGHT.

302. The method or combination of embodiment 282, wherein the secondary T-cell signaling molecule is OX40.

303. The method or combination of embodiment 282, wherein the secondary T-cell signaling molecule is PD1.

304. The method or combination of embodiment 282, wherein the secondary T-cell signaling molecule is PDL1.

305. The method or combination of embodiment 282, wherein the secondary T-cell signaling molecule is PDL2.

306. The method or combination of embodiment 282, wherein the secondary T-cell signaling molecule is TIGIT.

307. The method or combination of embodiment 282, wherein the secondary T-cell signaling molecule is TIM1.

308. The method or combination of embodiment 282, wherein the secondary T-cell signaling molecule is TIM2.

309. The method or combination of embodiment 282, wherein the secondary T-cell signaling molecule is TIM3.

310. The method or combination of embodiment 282, wherein the secondary T-cell signaling molecule is VISTA.

311. The method or combination of embodiment 282, wherein the secondary T-cell signaling molecule is CD70.

312. The method or combination of embodiment 282, wherein the secondary T-cell signaling molecule is 4-1BB.

313. The method or combination of any one of embodiments 282 to 312, wherein ABM4 comprises the CDR sequences of an antibody set forth in Table 24.

314. The method or combination of embodiment 313, wherein ABM4 comprises the heavy and light chain variable region sequences of an antibody set forth in Table 24.

315. The method or combination of any one of embodiments 1 to 314, wherein the first MBM comprises an ABM2 and/or the second MBM comprises an ABM5.

316. The method or combination of embodiment 315, wherein the first MBM comprises an ABM2.

317. The method or combination of embodiment 316, wherein ABM2 is a non-immunoglobulin scaffold based ABM.

318. The method or combination of embodiment 317, wherein ABM2 is a Kunitz domain, an Adnexin, an Affibody, a DARPin, an Avimer, an Anticalin, a Lipocalin, a Centyrin, a Versabody, a Knottin, an Adnectin, a Pronectin, an Affitin/Nanofitin, an Affilin, an Atrimer/Tetranectin, a bicyclic peptide, a cys-knot, a Fn3 scaffold, an Obody, a Tn3, an Affimer, BD, an Adhiron, a Duocalin, an Alphabody, an Armadillo Repeat Protein, a Repebody, or a Fynomer.

319. The method or combination of embodiment 316, wherein ABM2 is an immunoglobulin scaffold based ABM.

320. The method or combination of embodiment 319, wherein ABM2 is an antibody, an antibody fragment, an scFv, a dsFv, a Fv, a Fab, an scFab, a (Fab′)2, a single domain antibody (SDAB), a VH or VL domain, or a camelid VHH domain.

321. The method or combination of embodiment 320, wherein ABM2 is an antibody or an antigen-binding domain thereof.

322. The method or combination of embodiment 321, wherein ABM2 is an scFv.

323. The method or combination of embodiment 321, wherein ABM2 is a Fab.

324. The method or combination of embodiment 323, wherein ABM2 is a Fab heterodimer.

325. The method or combination of any one of embodiments 315 to 324, wherein the second MBM comprises an ABM5.

326. The method or combination of embodiment 325, wherein ABM5 is a non-immunoglobulin scaffold based ABM.

327. The method or combination of embodiment 326, wherein ABM5 is a Kunitz domain, an Adnexin, an Affibody, a DARPin, an Avimer, an Anticalin, a Lipocalin, a Centyrin, a Versabody, a Knottin, an Adnectin, a Pronectin, an Affitin/Nanofitin, an Affilin, an Atrimer/Tetranectin, a bicyclic peptide, a cys-knot, a Fn3 scaffold, an Obody, a Tn3, an Affimer, BD, an Adhiron, a Duocalin, an Alphabody, an Armadillo Repeat Protein, a Repebody, or a Fynomer.

328. The method or combination of embodiment 325, wherein ABM5 is an immunoglobulin scaffold based ABM.

329. The method or combination of embodiment 328, wherein ABM5 is an antibody, an antibody fragment, an scFv, a dsFv, a Fv, a Fab, an scFab, a (Fab′)2, a single domain antibody (SDAB), a VH or VL domain, or a camelid VHH domain.

330. The method or combination of embodiment 329, wherein ABM5 is an antibody or an antigen-binding domain thereof.

331. The method or combination of embodiment 330, wherein ABM5 is an scFv.

332. The method or combination of embodiment 330, wherein ABM5 is a Fab.

333. The method or combination of embodiment 332, wherein ABM5 is a Fab heterodimer.

334. The method or combination of any one of embodiments 315 to 333, wherein the first MBM comprises an ABM2 and the second MBM comprises an ABM5.

335. The method or combination of embodiment 334, wherein ABM2 and ABM5 bind specifically to the same TAA.

336. The method or combination of embodiment 335, wherein the TAA is CD19, CD20, CD22, CD123, BCMA, CD33, CLL-1, CD138, CS1, CD38, CD133, FLT3, CD52, ENPP1, TNFRSF13C, TNFRSF13B, CXCR4, PD-L1, LY9, CD200, FCGR2B, CD21, CD23, CD24, CD40L, CD72, CD74, CD79a, CD79b, CD93, or CD99.

337. The method or combination of embodiment 336, wherein the TAA is CD19.

338. The method or combination of embodiment 336, wherein the TAA is CD20.

339. The method or combination of embodiment 336, wherein the TAA is CD22.

340. The method or combination of embodiment 336, wherein the TAA is CD123.

341. The method or combination of embodiment 336, wherein the TAA is BCMA.

342. The method or combination of embodiment 336, wherein the TAA is CD33.

343. The method or combination of embodiment 336, wherein the TAA is CLL1.

344. The method or combination of embodiment 336, wherein the TAA is CD138.

345. The method or combination of embodiment 336, wherein the TAA is CS1.

346. The method or combination of embodiment 336, wherein the TAA is CD38.

347. The method or combination of embodiment 336, wherein the TAA is CD133.

348. The method or combination of embodiment 336, wherein the TAA is FLT3.

349. The method or combination of embodiment 336, wherein the TAA is CD52.

350. The method or combination of embodiment 336, wherein the TAA is ENPP1.

351. The method or combination of embodiment 336, wherein the TAA is TNFRSF13C.

352. The method or combination of embodiment 336, wherein the TAA is TNFRSF13B.

353. The method or combination of embodiment 336, wherein the TAA is CXCR4.

354. The method or combination of embodiment 336, wherein the TAA is PD-L1.

355. The method or combination of embodiment 336, wherein the TAA is LY9.

356. The method or combination of embodiment 336, wherein the TAA is CD200.

357. The method or combination of embodiment 336, wherein the TAA is FCGR2B.

358. The method or combination of embodiment 336, wherein the TAA is CD21.

359. The method or combination of embodiment 336, wherein the TAA is CD23.

360. The method or combination of embodiment 336, wherein the TAA is CD24.

361. The method or combination of embodiment 336, wherein the TAA is CD40L.

362. The method or combination of embodiment 336, wherein the TAA is CD72.

363. The method or combination of embodiment 336, wherein the TAA is CD74.

364. The method or combination of embodiment 336, wherein the TAA is CD79a.

365. The method or combination of embodiment 336, wherein the TAA is CD79b.

366. The method or combination of embodiment 336, wherein the TAA is CD93.

367. The method or combination of embodiment 336, wherein the TAA is CD99.

368. The method or combination of embodiment 335, wherein the TAA is mesothelin, TSHR, CD171, CS-1, CLL-1, GD3, Tn Ag, FLT3, CD38, CD44v6, B7H3, KIT, IL-13Ra2, IL-11Ra, PSCA, PRSS21, VEGFR2, LewisY, CD24, PDGFR-beta, SSEA-4, MUC1, EGFR, NCAM, CAIX, LMP2, EphA2, fucosyl GM1, sLe, GM3, TGS5, HMWMAA, o-acetyl-GD2, GD2, folate receptor alpha, folate receptor beta, TEM1/CD248, TEM7R, CLDN6, GPRC5D, CXORF61, CD97, CD179a, ALK, polysialic acid, PLAC1, GloboH, NY-BR-1, UPK2, HAVCR1, ADRB3, PANX3, GPR20, LY6K, OR51E2, TAARP, WT1, ETV6-AML, sperm protein 17, XAGE1, Tie 2, MAD-CT-1, MAD-CT-2, Fos-related antigen 1, p53 mutant, hTERT, sarcoma translocation breakpoints, ML-IAP, ERG (TMPRSS2 ETS fusion gene), NA17, PAX3, Androgen receptor, Cyclin B1, MYCN, RhoC, CYP1B1, BORIS, SART3, PAX5, OY-TES1, LCK, AKAP-4, SSX2, CD79a, CD79b, CD72, LAIR1, FCAR, LILRA2, CD300LF, CLEC12A, BST2, EMR2, LY75, GPC3, FCRL5, IGLL1, CD19, CD20, CD30, ERBB2, ROR1, TAAG72, CD22, GD2, BCMA, gp100Tn, FAP, tyrosinase, EPCAM, CEA, Igf-I receptor, EphB2, Cadherin17, CD32b, EGFRvIII, GPNMB, GPR64, HER3, LRP6, LYPD8, NKG2D, SLC34A2, SLC39A6, SLITRK6, TACSTD2, CD123, CD33, CD138, CS1, CD133, CD52, TNFRSF13C, TNFRSF13B, CXCR4, PD-L1, LY9, CD200, FCGR2B, CD21, CD23, or CD40L.

369. The method or combination of embodiment 368, wherein the TAA is mesothelin.

370. The method or combination of embodiment 368, wherein the TAA is TSHR.

371. The method or combination of embodiment 368, wherein the TAA is CD171.

372. The method or combination of embodiment 368, wherein the TAA is CS-1.

373. The method or combination of embodiment 368, wherein the TAA is GD3.

374. The method or combination of embodiment 368, wherein the TAA is Tn Ag.

375. The method or combination of embodiment 368, wherein the TAA is CD44v6.

376. The method or combination of embodiment 368, wherein the TAA is B7H3.

377. The method or combination of embodiment 368, wherein the TAA is KIT.

378. The method or combination of embodiment 368, wherein the TAA is IL-13Ra2.

379. The method or combination of embodiment 368, wherein the TAA is IL-11Ra.

380. The method or combination of embodiment 368, wherein the TAA is PSCA.

381. The method or combination of embodiment 368, wherein the TAA is PRSS21.

382. The method or combination of embodiment 368, wherein the TAA is VEGFR2.

383. The method or combination of embodiment 368, wherein the TAA is LewisY.

384. The method or combination of embodiment 368, wherein the TAA is PDGFR-beta.

385. The method or combination of embodiment 368, wherein the TAA is SSEA-4.

386. The method or combination of embodiment 368, wherein the TAA is MUC1.

387. The method or combination of embodiment 368, wherein the TAA is EGFR.

388. The method or combination of embodiment 368, wherein the TAA is NCAM.

389. The method or combination of embodiment 368, wherein the TAA is CAIX.

390. The method or combination of embodiment 368, wherein the TAA is LM P2.

391. The method or combination of embodiment 368, wherein the TAA is EphA2.

392. The method or combination of embodiment 368, wherein the TAA is fucosyl GM1.

393. The method or combination of embodiment 368, wherein the TAA is sLe.

394. The method or combination of embodiment 368, wherein the TAA is GM3.

395. The method or combination of embodiment 368, wherein the TAA is TGS5.

396. The method or combination of embodiment 368, wherein the TAA is HMWMAA.

397. The method or combination of embodiment 368, wherein the TAA is o-acetyl-GD2.

398. The method or combination of embodiment 368, wherein the TAA is GD2.

399. The method or combination of embodiment 368, wherein the TAA is folate receptor alpha.

400. The method or combination of embodiment 368, wherein the TAA is folate receptor beta.

401. The method or combination of embodiment 368, wherein the TAA is TEM1/CD248.

402. The method or combination of embodiment 368, wherein the TAA is TEM7R.

403. The method or combination of embodiment 368, wherein the TAA is CLDN6.

404. The method or combination of embodiment 368, wherein the TAA is GPRC5D.

405. The method or combination of embodiment 368, wherein the TAA is CXORF61.

406. The method or combination of embodiment 368, wherein the TAA is CD97.

407. The method or combination of embodiment 368, wherein the TAA is CD179a.

408. The method or combination of embodiment 368, wherein the TAA is ALK.

409. The method or combination of embodiment 368, wherein the TAA is polysialic acid.

410. The method or combination of embodiment 368, wherein the TAA is PLAC1.

411. The method or combination of embodiment 368, wherein the TAA is GloboH.

412. The method or combination of embodiment 368, wherein the TAA is NY-BR-1.

413. The method or combination of embodiment 368, wherein the TAA is UPK2.

414. The method or combination of embodiment 368, wherein the TAA is HAVCR1.

415. The method or combination of embodiment 368, wherein the TAA is ADRB3.

416. The method or combination of embodiment 368, wherein the TAA is PANX3.

417. The method or combination of embodiment 368, wherein the TAA is GPR20.

418. The method or combination of embodiment 368, wherein the TAA is LY6K.

419. The method or combination of embodiment 368, wherein the TAA is OR51E2.

420. The method or combination of embodiment 368, wherein the TAA is TAARP.

421. The method or combination of embodiment 368, wherein the TAA is WT1.

422. The method or combination of embodiment 368, wherein the TAA is ETV6-AML.

423. The method or combination of embodiment 368, wherein the TAA is sperm protein 17.

424. The method or combination of embodiment 368, wherein the TAA is XAGE1.

425. The method or combination of embodiment 368, wherein the TAA is Tie 2.

426. The method or combination of embodiment 368, wherein the TAA is MAD-CT-1.

427. The method or combination of embodiment 368, wherein the TAA is MAD-CT-2.

428. The method or combination of embodiment 368, wherein the TAA is Fos-related antigen 1.

429. The method or combination of embodiment 368, wherein the TAA is p53 mutant.

430. The method or combination of embodiment 368, wherein the TAA is hTERT.

431. The method or combination of embodiment 368, wherein the TAA is sarcoma translocation breakpoints.

432. The method or combination of embodiment 368, wherein the TAA is ML-IAP.

433. The method or combination of embodiment 368, wherein the TAA is ERG (TMPRSS2 ETS fusion gene).

434. The method or combination of embodiment 368, wherein the TAA is NA17.

435. The method or combination of embodiment 368, wherein the TAA is PAX3.

436. The method or combination of embodiment 368, wherein the TAA is Androgen receptor.

437. The method or combination of embodiment 368, wherein the TAA is Cyclin B1.

438. The method or combination of embodiment 368, wherein the TAA is MYCN.

439. The method or combination of embodiment 368, wherein the TAA is RhoC.

440. The method or combination of embodiment 368, wherein the TAA is CYP1B1.

441. The method or combination of embodiment 368, wherein the TAA is BORIS.

442. The method or combination of embodiment 368, wherein the TAA is SART3.

443. The method or combination of embodiment 368, wherein the TAA is PAX5.

444. The method or combination of embodiment 368, wherein the TAA is OY-TES1.

445. The method or combination of embodiment 368, wherein the TAA is LCK.

446. The method or combination of embodiment 368, wherein the TAA is AKAP-4.

447. The method or combination of embodiment 368, wherein the TAA is SSX2.

448. The method or combination of embodiment 368, wherein the TAA is LAIR1.

449. The method or combination of embodiment 368, wherein the TAA is FCAR.

450. The method or combination of embodiment 368, wherein the TAA is LILRA2.

451. The method or combination of embodiment 368, wherein the TAA is CD300LF.

452. The method or combination of embodiment 368, wherein the TAA is CLEC12A.

453. The method or combination of embodiment 368, wherein the TAA is BST2.

454. The method or combination of embodiment 368, wherein the TAA is EMR2.

455. The method or combination of embodiment 368, wherein the TAA is LY75.

456. The method or combination of embodiment 368, wherein the TAA is GPC3.

457. The method or combination of embodiment 368, wherein the TAA is FCRL5.

458. The method or combination of embodiment 368, wherein the TAA is IGLL1.

459. The method or combination of embodiment 368, wherein the TAA is CD30.

460. The method or combination of embodiment 368, wherein the TAA is ERBB2.

461. The method or combination of embodiment 368, wherein the TAA is ROR1.

462. The method or combination of embodiment 368, wherein the TAA is TAAG72.

463. The method or combination of embodiment 368, wherein the TAA is GD2.

464. The method or combination of embodiment 368, wherein the TAA is gp100Tn.

465. The method or combination of embodiment 368, wherein the TAA is FAP.

466. The method or combination of embodiment 368, wherein the TAA is tyrosinase.

467. The method or combination of embodiment 368, wherein the TAA is EPCAM.

468. The method or combination of embodiment 368, wherein the TAA is CEA.

469. The method or combination of embodiment 368, wherein the TAA is Igf-I receptor.

470. The method or combination of embodiment 368, wherein the TAA is EphB2.

471. The method or combination of embodiment 368, wherein the TAA is Cadherin17.

472. The method or combination of embodiment 368, wherein the TAA is CD32b.

473. The method or combination of embodiment 368, wherein the TAA is EGFRvIII.

474. The method or combination of embodiment 368, wherein the TAA is GPNMB.

475. The method or combination of embodiment 368, wherein the TAA is GPR64.

476. The method or combination of embodiment 368, wherein the TAA is HER3.

477. The method or combination of embodiment 368, wherein the TAA is LRP6.

478. The method or combination of embodiment 368, wherein the TAA is LYPD8.

479. The method or combination of embodiment 368, wherein the TAA is NKG2D.

480. The method or combination of embodiment 368, wherein the TAA is SLC34A2.

481. The method or combination of embodiment 368, wherein the TAA is SLC39A6.

482. The method or combination of embodiment 368, wherein the TAA is SLITRK6.

483. The method or combination of embodiment 368, wherein the TAA is TACSTD2.

484. The method or combination of any one of embodiments 335 to 483, wherein ABM2 and ABM5 bind specifically to different epitopes on the same TAA.

485. The method or combination of embodiment 484, wherein the different epitopes do not overlap.

486. The method or combination of any one of embodiments 335 to 485, wherein the first MBM and second MBM are capable of specifically binding the TAA simultaneously.

487. The method or combination of any one of embodiments 335 to 486, wherein binding of the first MBM to the TAA reduces binding of the second MBM to the TAA by less than 50% in a competition assay.

488. The method or combination of any one of embodiments 335 to 486, wherein binding of the first MBM to the TAA reduces binding of the second MBM to the TAA by less than 40% in a competition assay.

489. The method or combination of any one of embodiments 335 to 486, wherein binding of the first MBM to the TAA reduces binding of the second MBM to the TAA by less than 30% in a competition assay.

490. The method or combination of any one of embodiments 335 to 486, wherein binding of the first MBM to the TAA reduces binding of the second MBM to the TAA by less than 20% in a competition assay.

491. The method or combination of any one of embodiments 335 to 486, wherein binding of the first MBM to the TAA reduces binding of the second MBM to the TAA by less than 10% in a competition assay.

492. The method or combination of any one of embodiments 487 to 491, wherein the competition assay is an ELISA assay, a Biacore assay, a FACS assay.

493. The method or combination of any one of embodiments 315 to 334, wherein ABM2, when present in the first MBM, binds specifically to a first TAA (“TAA 1”) and ABM5, when present in the second MBM, binds specifically to a second TAA (“TAA 2”), and wherein when ABM2 is present in the first MBM and ABM5 is present in the second MBM, TAA 1 and TAA 2 are different TAAs.

494. The method or combination of embodiment 493, wherein TAA 1 and TAA 2 are selected from CD19, CD20, CD22, CD123, BCMA, CD33, CLL-1, CD138, CS1, CD38, CD133, FLT3, CD52, ENPP1, TNFRSF13C, TNFRSF13B, CXCR4, PD-L1, LY9, CD200, FCGR2B, CD21, CD23, CD24, CD40L, CD72, CD74, CD79a, CD79b, CD93, and CD99.

495. The method or combination of embodiment 493 or 494, wherein TAA 1 is CD19.

496. The method or combination of embodiment 493 or 494, wherein TAA 1 is CD20.

497. The method or combination of embodiment 493 or 494, wherein TAA 1 is CD22.

498. The method or combination of embodiment 493 or 494, wherein TAA 1 is CD123.

499. The method or combination of embodiment 493 or 494, wherein TAA 1 is BCMA.

500. The method or combination of embodiment 493 or 494, wherein TAA 1 is CD33.

501. The method or combination of embodiment 493 or 494, wherein TAA 1 is CLL1.

502. The method or combination of embodiment 493 or 494, wherein TAA 1 is CD138.

503. The method or combination of embodiment 493 or 494, wherein TAA 1 is CS1.

504. The method or combination of embodiment 493 or 494, wherein TAA 1 is CD38.

505. The method or combination of embodiment 493 or 494, wherein TAA 1 is CD133.

506. The method or combination of embodiment 493 or 494, wherein TAA 1 is FLT3.

507. The method or combination of embodiment 493 or 494, wherein TAA 1 is CD52.

508. The method or combination of embodiment 493 or 494, wherein TAA 1 is ENPP1.

509. The method or combination of embodiment 493 or 494, wherein TAA 1 is TNFRSF13C.

510. The method or combination of embodiment 493 or 494, wherein TAA 1 is CXC R4.

511. The method or combination of embodiment 493 or 494, wherein TAA 1 is PD-L1.

512. The method or combination of embodiment 493 or 494, wherein TAA 1 is LY9.

513. The method or combination of embodiment 493 or 494, wherein TAA 1 is CD200.

514. The method or combination of embodiment 493 or 494, wherein TAA 1 is FCGR2B.

515. The method or combination of embodiment 493 or 494, wherein TAA 1 is CD24.

516. The method or combination of embodiment 493 or 494, wherein TAA 1 is CD40L.

517. The method or combination of embodiment 493 or 494, wherein TAA 1 is CD72.

518. The method or combination of embodiment 493 or 494, wherein TAA 1 is CD74.

519. The method or combination of embodiment 493 or 494, wherein TAA 1 is CD79a.

520. The method or combination of embodiment 493 or 494, wherein TAA 1 is CD93.

521. The method or combination of embodiment 493 or 494, wherein TAA 1 is CD99.

522. The method or combination of any one of embodiments 493 to 521 other than embodiments where TAA 1 is CD19, wherein TAA 2 is CD19.

523. The method or combination of any one of embodiments 493 to 521 other than embodiments where TAA 1 is CD20, wherein TAA 2 is CD20.

524. The method or combination of any one of embodiments 493 to 521 other than embodiments where TAA 1 is CD22, wherein TAA 2 is CD22.

525. The method or combination of any one of embodiments 493 to 521 other than embodiments where TAA 1 is CD123, wherein TAA 2 is CD123.

526. The method or combination of any one of embodiments 493 to 521 other than embodiments where TAA 1 is BCMA, wherein TAA 2 is BCMA.

527. The method or combination of any one of embodiments 493 to 521 other than embodiments where TAA 1 is CD33, wherein TAA 2 is CD33.

528. The method or combination of any one of embodiments 493 to 521 other than embodiments where TAA 1 is CLL1, wherein TAA 2 is CLL1.

529. The method or combination of any one of embodiments 493 to 521 other than embodiments where TAA 1 is CD138, wherein TAA 2 is CD138.

530. The method or combination of any one of embodiments 493 to 521 other than embodiments where TAA 1 is CS1, wherein TAA 2 is CS1.

531. The method or combination of any one of embodiments 493 to 521 other than embodiments where TAA 1 is CD38, wherein TAA 2 is CD38.

532. The method or combination of any one of embodiments 493 to 521 other than embodiments where TAA 1 is CD133, wherein TAA 2 is CD133.

533. The method or combination of any one of embodiments 493 to 521 other than embodiments where TAA 1 is FLT3, wherein TAA 2 is FLT3.

534. The method or combination of any one of embodiments 493 to 521 other than embodiments where TAA 1 is CD52, wherein TAA 2 is CD52.

535. The method or combination of any one of embodiments 493 to 521 other than embodiments where TAA 1 is ENPP1, wherein TAA 2 is ENPP1.

536. The method or combination of any one of embodiments 493 to 521 other than embodiments where TAA 1 is TNFRSF13C, wherein TAA 2 is TNFRSF13C.

537. The method or combination of any one of embodiments 493 to 521 other than embodiments where TAA 1 is CXCR4, wherein TAA 2 is CXCR4.

538. The method or combination of any one of embodiments 493 to 521 other than embodiments where TAA 1 is PD-L1, wherein TAA 2 is PD-L1.

539. The method or combination of any one of embodiments 493 to 521 other than embodiments where TAA 1 is LY9, wherein TAA 2 is LY9.

540. The method or combination of any one of embodiments 493 to 521 other than embodiments where TAA 1 is CD200, wherein TAA 2 is CD200.

541. The method or combination of any one of embodiments 493 to 521 other than embodiments where TAA 1 is FCGR2B, wherein TAA 2 is FCGR2B.

542. The method or combination of any one of embodiments 493 to 521 other than embodiments where TAA 1 is CD24, wherein TAA 2 is CD24.

543. The method or combination of any one of embodiments 493 to 521 other than embodiments where TAA 1 is CD40L, wherein TAA 2 is CD40L.

544. The method or combination of any one of embodiments 493 to 521 other than embodiments where TAA 1 is CD72, wherein TAA 2 is CD72.

545. The method or combination of any one of embodiments 493 to 521 other than embodiments where TAA 1 is CD74, wherein TAA 2 is CD74.

546. The method or combination of any one of embodiments 493 to 521 other than embodiments where TAA 1 is CD79a, wherein TAA 2 is CD79a.

547. The method or combination of any one of embodiments 493 to 521 other than embodiments where TAA 1 is CD93, wherein TAA 2 is CD93.

548. The method or combination of any one of embodiments 493 to 521 other than embodiments where TAA 1 is CD99, wherein TAA 2 is CD99.

549. The method or combination of embodiment 493, wherein the TAA 1 and TAA 2 are selected from mesothelin, TSHR, CD171, CS-1, CLL-1, GD3, Tn Ag, FLT3, CD38, CD44v6, B7H3, KIT, IL-13Ra2, IL-11Ra, PSCA, PRSS21, VEGFR2, LewisY, CD24, PDGFR-beta, SSEA-4, MUC1, EGFR, NCAM, CAIX, LMP2, EphA2, fucosyl GM1, sLe, GM3, TGS5, HMWMAA, o-acetyl-GD2, GD2, folate receptor alpha, folate receptor beta, TEM1/CD248, TEM7R, CLDN6, GPRC5D, CXORF61, CD97, CD179a, ALK, polysialic acid, PLAC1, GloboH, NY-BR-1, UPK2, HAVCR1, ADRB3, PANX3, GPR20, LY6K, OR51E2, TAARP, WT1, ETV6-AML, sperm protein 17, XAGE1, Tie 2, MAD-CT-1, MAD-CT-2, Fos-related antigen 1, p53 mutant, hTERT, sarcoma translocation breakpoints, ML-IAP, ERG (TMPRSS2 ETS fusion gene), NA17, PAX3, Androgen receptor, Cyclin B1, MYCN, RhoC, CYP1B1, BORIS, SART3, PAX5, OY-TES1, LCK, AKAP-4, SSX2, CD79a, CD79b, CD72, LAIR1, FCAR, LILRA2, CD300LF, CLEC12A, BST2, EMR2, LY75, GPC3, FCRL5, IGLL1, CD19, CD20, CD30, ERBB2, ROR1, TAAG72, CD22, GD2, BCMA, gp100Tn, FAP, tyrosinase, EPCAM, CEA, Igf-I receptor, EphB2, Cadherin17, CD32b, EGFRvIII, GPNMB, GPR64, HER3, LRP6, LYPD8, NKG2D, SLC34A2, SLC39A6, SLITRK6, TACSTD2, CD123, CD33, CD138, CS1, CD133, CD52, TNFRSF13C, TNFRSF13B, CXCR4, PD-L1, LY9, CD200, FCGR2B, CD21, CD23, and CD40L.

550. The method or combination of any one of embodiments 493 to 549, wherein TAA 1 and TAA 2 are expressed on the same cell.

551. The method or combination of any one of embodiments 493 to 549, wherein TAA 1 and TAA 2 are expressed on different cells.

552. The method or combination of any one of embodiments 315 to 551, wherein when ABM2 and/or ABM5 binds specifically to CD19, ABM2 or ABM5 comprises the CDR-H1, CDR-H2, CDR-H3, CDR-L1, CDR-L2, and CDR-L3 sequence of the anti-CD19 antibody NEG258 as defined by Kabat and set forth in Table 17A.

553. The method or combination of any one of embodiments 315 to 551, wherein when ABM2 and/or ABM5 binds specifically to CD19, ABM2 or ABM5 comprises the CDR-H1, CDR-H2, CDR-H3, CDR-L1, CDR-L2, and CDR-L3 sequences of the anti-CD19 antibody NEG258 as defined by Chothia and set forth in Table 17A.

554. The method or combination of any one of embodiments 315 to 551, wherein when ABM2 and/or ABM5 binds specifically to CD19, ABM2 or ABM5 comprises the CDR-H1, CDR-H2, CDR-H3, CDR-L1, CDR-L2, and CDR-L3 sequence of the anti-CD19 antibody NEG258 as defined by IMGT and set forth in Table 17A.

555. The method or combination of any one of embodiments 315 to 551, wherein when ABM2 and/or ABM5 binds specifically to CD19, ABM2 or ABM5 comprises the CDR-H1, CDR-H2, CDR-H3, CDR-L1, CDR-L2, and CDR-L3 sequences of the anti-CD19 antibody NEG258 as defined by the combination of Kabat and Chothia and set forth in Table 17A.

556. The method or combination of any one of embodiments 315 to 551, wherein when ABM2 and/or ABM5 binds specifically to CD19, ABM2 or ABM5 comprises the heavy chain and/or light chain variable sequences of the anti-CD19 antibody NEG258 as set forth in Table 17A.

557. The method or combination of any one of embodiments 315 to 551, wherein when ABM2 and/or ABM5 binds specifically to CD19, ABM2 or ABM5 comprises the CDR-H1, CDR-H2, CDR-H3, CDR-L1, CDR-L2, and CDR-L3 sequence of the anti-CD19 antibody NEG218 as defined by Kabat and set forth in Table 17B.

558. The method or combination of any one of embodiments 315 to 551, wherein when ABM2 and/or ABM5 binds specifically to CD19, ABM2 or ABM5 comprises the CDR-H1, CDR-H2, CDR-H3, CDR-L1, CDR-L2, and CDR-L3 sequences of the anti-CD19 antibody NEG218 as defined by Chothia and set forth in Table 17B.

559. The method or combination of any one of embodiments 315 to 551, wherein when ABM2 and/or ABM5 binds specifically to CD19, ABM2 or ABM5 comprises the CDR-H1, CDR-H2, CDR-H3, CDR-L1, CDR-L2, and CDR-L3 sequence of the anti-CD19 antibody NEG218 as defined by IMGT and set forth in Table 17B.

560. The method or combination of any one of embodiments 315 to 551, wherein when ABM2 and/or ABM5 binds specifically to CD19, ABM2 or ABM5 comprises the CDR-H1, CDR-H2, CDR-H3, CDR-L1, CDR-L2, and CDR-L3 sequences of the anti-CD19 antibody NEG218 as defined by the combination of Kabat and Chothia and set forth in Table 17B.

561. The method or combination of any one of embodiments 315 to 551, wherein when ABM2 and/or ABM5 binds specifically to CD19, ABM2 or ABM5 comprises the heavy chain and/or light chain variable sequences of the anti-CD19 antibody NEG218 as set forth in Table 17B.

562. The method or combination of any one of embodiments 315 to 551, wherein when ABM2 and/or ABM5 binds specifically to CD19, ABM2 or ABM5 comprises heavy chain CDRs having the amino acid sequences of CD19-H1, CD19-H2A, and CD19-H3 as set forth in Table 16 and light chain CDRs having the amino acid sequences of CD19-L1, CD19-L2, and CD19-L3 as set forth in Table 16.

563. The method or combination of any one of embodiments 315 to 551, wherein when ABM2 and/or ABM5 binds specifically to CD19, ABM2 or ABM5 comprises a heavy chain variable region having the amino acid sequences of VHA as set forth in Table 16 and a light chain variable region having the amino acid sequences of VLA as set forth in Table 16.

564. The method or combination of any one of embodiments 315 to 551, wherein when ABM2 and/or ABM5 binds specifically to CD19, ABM2 or ABM5 comprises heavy chain CDRs having the amino acid sequences of CD19-H1, CD19-H2B, and CD19-H3 as set forth in Table 16 and light chain CDRs having the amino acid sequences of CD19-L1, CD19-L2, and CD19-L3 as set forth in Table 16.

565. The method or combination of any one of embodiments 315 to 551, wherein when ABM2 and/or ABM5 binds specifically to CD19, ABM2 or ABM5 comprises a heavy chain variable region having the amino acid sequences of VHB as set forth in Table 16 and a light chain variable region having the amino acid sequences of VLB as set forth in Table 16.

566. The method or combination of any one of embodiments 315 to 551, wherein when ABM2 and/or ABM5 binds specifically to CD19, ABM2 or ABM5 comprises heavy chain CDRs having the amino acid sequences of CD19-H1, CD19-H2C, and CD19-H3 as set forth in Table 16 and light chain CDRs having the amino acid sequences of CD19-L1, CD19-L2, and CD19-L3 as set forth in Table 16.

567. The method or combination of any one of embodiments 315 to 551, wherein when ABM2 and/or ABM5 binds specifically to CD19, ABM2 or ABM5 comprises a heavy chain variable region having the amino acid sequences of VHC as set forth in Table 16 and a light chain variable region having the amino acid sequences of VLB as set forth in Table 16.

568. The method or combination of any one of embodiments 315 to 551, wherein when ABM2 and/or ABM5 binds specifically to CD19, ABM2 or ABM5 comprises heavy chain CDRs having the amino acid sequences of CD19-H1, CD19-H2D, and CD19-H3 as set forth in Table 16 and light chain CDRs having the amino acid sequences of CD19-L1, CD19-L2, and CD19-L3 as set forth in Table 16.

569. The method or combination of any one of embodiments 315 to 551, wherein when ABM2 and/or ABM5 binds specifically to CD19, ABM2 or ABM5 comprises a heavy chain variable region having the amino acid sequences of VHD as set forth in Table 16 and a light chain variable region having the amino acid sequences of VLB as set forth in Table 16.

570. The method or combination of any one of embodiments 315 to 551, wherein when ABM2 and/or ABM5 binds specifically to CD19, ABM2 or ABM5 comprises a heavy chain variable region having the amino acid sequences of VHE as set forth in Table 16 and a light chain variable region having the amino acid sequences of VLE as set forth in Table 16.

571. The method or combination of any one of embodiments 315 to 551, wherein when ABM2 and/or ABM5 binds specifically to CD19, ABM2 or ABM5 comprises a scFv comprising the amino acid sequence of CD19-scFv1 as set forth in Table 16.

572. The method or combination of any one of embodiments 315 to 551, wherein when ABM2 and/or ABM5 binds specifically to CD19, ABM2 or ABM5 comprises a scFv comprising the amino acid sequence of CD19-scFv2 as set forth in Table 16.

573. The method or combination of any one of embodiments 315 to 551, wherein when ABM2 and/or ABM5 binds specifically to CD19, ABM2 or ABM5 comprises a scFv comprising the amino acid sequence of CD19-scFv3 as set forth in Table 16.

574. The method or combination of any one of embodiments 315 to 551, wherein when ABM2 and/or ABM5 binds specifically to CD19, ABM2 or ABM5 comprises a scFv comprising the amino acid sequence of CD19-scFv4 as set forth in Table 16.

575. The method or combination of any one of embodiments 315 to 551, wherein when ABM2 and/or ABM5 binds specifically to CD19, ABM2 or ABM5 comprises a scFv comprising the amino acid sequence of CD19-scFv5 as set forth in Table 16.

576. The method or combination of any one of embodiments 315 to 551, wherein when ABM2 and/or ABM5 binds specifically to CD19, ABM2 or ABM5 comprises a scFv comprising the amino acid sequence of CD19-scFv6 as set forth in Table 16.

577. The method or combination of any one of embodiments 315 to 551, wherein when ABM2 and/or ABM5 binds specifically to CD19, ABM2 or ABM5 comprises a scFv comprising the amino acid sequence of CD19-scFv7 as set forth in Table 16.

578. The method or combination of any one of embodiments 315 to 551, wherein when ABM2 and/or ABM5 binds specifically to CD19, ABM2 or ABM5 comprises a scFv comprising the amino acid sequence of CD19-scFv8 as set forth in Table 16.

579. The method or combination of any one of embodiments 315 to 551, wherein when ABM2 and/or ABM5 binds specifically to CD19, ABM2 or ABM5 comprises a scFv comprising the amino acid sequence of CD19-scFv9 as set forth in Table 16.

580. The method or combination of any one of embodiments 315 to 551, wherein when ABM2 and/or ABM5 binds specifically to CD19, ABM2 or ABM5 comprises a scFv comprising the amino acid sequence of CD19-scFv10 as set forth in Table 16.

581. The method or combination of any one of embodiments 315 to 551, wherein when ABM2 and/or ABM5 binds specifically to CD19, ABM2 or ABM5 comprises a scFv comprising the amino acid sequence of CD19-scFv11 as set forth in Table 16.

582. The method or combination of any one of embodiments 315 to 551, wherein when ABM2 and/or ABM5 binds specifically to CD19, ABM2 or ABM5 comprises a scFv comprising the amino acid sequence of CD19-scFv12 as set forth in Table 16.

583. The method or combination of any one of embodiments 315 to 551, wherein when ABM2 and/or ABM5 binds specifically to CD19, ABM2 or ABM5 comprises a scFv comprising the amino acid sequence of CD19-scFv13 as set forth in Table 16.

584. The method or combination of any one of embodiments 315 to 551, wherein when ABM2 and/or ABM5 binds specifically to CD19, ABM2 or ABM5 comprises a scFv comprising the amino acid sequence of CD19-scFv14 as set forth in Table 16.

585. The method or combination of any one of embodiments 315 to 551, wherein when ABM2 and/or ABM5 binds specifically to CD19, ABM2 or ABM5 comprises a scFv comprising the amino acid sequence of CD19-scFv15 as set forth in Table 16.

586. The method or combination of any one of embodiments 315 to 551, wherein when ABM2 and/or ABM5 binds specifically to CD19, ABM2 or ABM5 comprises a scFv comprising the amino acid sequence of CD19-scFv16 as set forth in Table 16.

587. The method or combination of any one of embodiments 315 to 586, wherein when ABM2 and/or ABM5 binds specifically to BCMA, ABM2 or ABM5 comprises CDR-L1, CDR-L2 and CDR-L3 sequences set forth in Table 15A-1, Table 15B-1, Table 15C-1, Table 15D-1, Table 15E-1, Table 15F-1, Table 15G-1, Table 15H-1, Table 15I-1, Table 15J-1, Table 15K-1(a), Table 15K-1(b), Table 15L-1, Table 15M-1, Table 15N-1(a), or Table 15N-1(b), and the corresponding CDR-H1, CDR-H2 and CDR-H3 sequence set forth in Table 15A-2, Table 15B-2, Table 15C-2, Table 15D-2, Table 15E-2, Table 15F-2, Table 15G-2, Table 15H-2, Table 15I-2, Table 15J-2, Table 15K-2, Table 15K-2, Table 15L-2, Table 15M-2, Table 15N-2, or Table 15N-2, respectively.

588. The method or combination of embodiment 587, wherein when ABM2 and/or ABM5 binds specifically to BCMA, ABM2 or ABM5 comprises CDR-L1, CDR-L2 and CDR-L3 sequences set forth in Table 15A-1, Table 15B-1, Table 15C-1, Table 15D-1, Table 15E-1, Table 15F-1, Table 15G-1, Table 15H-1, Table 15I-1, Table 15J-1, Table 15K-1(a), Table 15L-1, Table 15M-1, or Table 15N-1(a), and the corresponding CDR-H1, CDR-H2 and CDR-H3 sequence set forth in Table 15A-2, Table 15B-2, Table 15C-2, Table 15D-2, Table 15E-2, Table 15F-2, Table 15G-2, Table 15H-2, Table 15I-2, Table 15J-2, Table 15K-2, Table 15L-2, Table 15M-2, or Table 15N-2, respectively.

589. The method or combination of embodiment 587 or embodiment 588, wherein when ABM2 and/or ABM5 binds specifically to BCMA, ABM2 or ABM5 comprises CDR-L1, CDR-L2 and CDR-L3 sequences set forth in Table 15A-1 and the corresponding CDR-H1, CDR-H2 and CDR-H3 sequence set forth in Table 15A-2.

590. The method or combination of embodiment 587 or embodiment 588, wherein when ABM2 and/or ABM5 binds specifically to BCMA, ABM2 or ABM5 comprises CDR-L1, CDR-L2 and CDR-L3 sequences set forth in Table 15B-1 and the corresponding CDR-H1, CDR-H2 and CDR-H3 sequence set forth in Table 15B-2.

591. The method or combination of embodiment 587 or embodiment 588, wherein when ABM2 and/or ABM5 binds specifically to BCMA, ABM2 or ABM5 comprises CDR-L1, CDR-L2 and CDR-L3 sequences set forth in Table 15C-1 and the corresponding CDR-H1, CDR-H2 and CDR-H3 sequence set forth in Table 15C-2.

592. The method or combination of embodiment 587 or embodiment 588, wherein when ABM2 and/or ABM5 binds specifically to BCMA, ABM2 or ABM5 comprises CDR-L1, CDR-L2 and CDR-L3 sequences set forth in Table 15D-1 and the corresponding CDR-H1, CDR-H2 and CDR-H3 sequence set forth in Table 15D-2.

593. The method or combination of embodiment 587 or embodiment 588, wherein when ABM2 and/or ABM5 binds specifically to BCMA, ABM2 or ABM5 comprises CDR-L1, CDR-L2 and CDR-L3 sequences set forth in Table 15E-1 and the corresponding CDR-H1, CDR-H2 and CDR-H3 sequence set forth in Table 15E-2.

594. The method or combination of embodiment 587 or embodiment 588, wherein when ABM2 and/or ABM5 binds specifically to BCMA, ABM2 or ABM5 comprises CDR-L1, CDR-L2 and CDR-L3 sequences set forth in Table 15F-1 and the corresponding CDR-H1, CDR-H2 and CDR-H3 sequence set forth in Table 15F-2.

595. The method or combination of embodiment 587 or embodiment 588, wherein when ABM2 and/or ABM5 binds specifically to BCMA, ABM2 or ABM5 comprises CDR-L1, CDR-L2 and CDR-L3 sequences set forth in Table 15G-1 and the corresponding CDR-H1, CDR-H2 and CDR-H3 sequence set forth in Table 15G-2.

596. The method or combination of embodiment 587 or embodiment 588, wherein when ABM2 and/or ABM5 binds specifically to BCMA, ABM2 or ABM5 comprises CDR-L1, CDR-L2 and CDR-L3 sequences set forth in Table 15H-1 and the corresponding CDR-H1, CDR-H2 and CDR-H3 sequence set forth in Table 15H-2.

597. The method or combination of embodiment 587 or embodiment 588, wherein when ABM2 and/or ABM5 binds specifically to BCMA, ABM2 or ABM5 comprises CDR-L1, CDR-L2 and CDR-L3 sequences set forth in Table 15I-1 and the corresponding CDR-H1, CDR-H2 and CDR-H3 sequence set forth in Table 15I-2.

598. The method or combination of embodiment 587 or embodiment 588, wherein when ABM2 and/or ABM5 binds specifically to BCMA, ABM2 or ABM5 comprises CDR-L1, CDR-L2 and CDR-L3 sequences set forth in Table 15J-1 and the corresponding CDR-H1, CDR-H2 and CDR-H3 sequence set forth in Table 15J-2.

599. The method or combination of embodiment 587 or embodiment 588, wherein when ABM2 and/or ABM5 binds specifically to BCMA, ABM2 or ABM5 comprises CDR-L1, CDR-L2 and CDR-L3 sequences set forth in Table 15K-1(a) and the corresponding CDR-H1, CDR-H2 and CDR-H3 sequence set forth in Table 15K-2.

600. The method or combination of embodiment 587 or embodiment 588, wherein when ABM2 and/or ABM5 binds specifically to BCMA, ABM2 or ABM5 comprises CDR-L1, CDR-L2 and CDR-L3 sequences set forth in Table 15K-1(b) and the corresponding CDR-H1, CDR-H2 and CDR-H3 sequence set forth in Table 15K-2.

601. The method or combination of embodiment 587 or embodiment 588, wherein when ABM2 and/or ABM5 binds specifically to BCMA, ABM2 or ABM5 comprises CDR-L1, CDR-L2 and CDR-L3 sequences set forth in Table 15L-1 and the corresponding CDR-H1, CDR-H2 and CDR-H3 sequence set forth in Table 15L-2.

602. The method or combination of embodiment 587 or embodiment 588, wherein when ABM2 and/or ABM5 binds specifically to BCMA, ABM2 or ABM5 comprises CDR-L1, CDR-L2 and CDR-L3 sequences set forth in Table 15M-1 and the corresponding CDR-H1, CDR-H2 and CDR-H3 sequence set forth in Table 15M-2.

603. The method or combination of embodiment 587 or embodiment 588, wherein when ABM2 and/or ABM5 binds specifically to BCMA, ABM2 or ABM5 comprises CDR-L1, CDR-L2 and CDR-L3 sequences set forth in Table 15N-1(a) and the corresponding CDR-H1, CDR-H2 and CDR-H3 sequence set forth in Table 15N-2.

604. The method or combination of embodiment 587 or embodiment 588, wherein when ABM2 and/or ABM5 binds specifically to BCMA, ABM2 or ABM5 comprises CDR-L1, CDR-L2 and CDR-L3 sequences set forth in Table 15N-1(b) and the corresponding CDR-H1, CDR-H2 and CDR-H3 sequence set forth in Table 15N-2.

605. The method or combination of embodiment 589, wherein the CDR-L1, CDR-L2, CDR-L3, CDR-H1, CDR-H2 and CDR-H3 sequences are those of BCMA C1.

606. The method or combination of embodiment 589, wherein the CDR-L1, CDR-L2, CDR-L3, CDR-H1, CDR-H2 and CDR-H3 sequences are those of BCMA C2.

607. The method or combination of embodiment 589, wherein the CDR-L1, CDR-L2, CDR-L3, CDR-H1, CDR-H2 and CDR-H3 sequences are those of BCMA C3.

608. The method or combination of embodiment 589, wherein the CDR-L1, CDR-L2, CDR-L3, CDR-H1, CDR-H2 and CDR-H3 sequences are those of BCMA C4.

609. The method or combination of embodiment 589, wherein the CDR-L1, CDR-L2, CDR-L3, CDR-H1, CDR-H2 and CDR-H3 sequences are those of BCMA C5.

610. The method or combination of embodiment 589, wherein the CDR-L1, CDR-L2, CDR-L3, CDR-H1, CDR-H2 and CDR-H3 sequences are those of BCMA C6.

611. The method or combination of embodiment 589, wherein the CDR-L1, CDR-L2, CDR-L3, CDR-H1, CDR-H2 and CDR-H3 sequences are those of BCMA C7.

612. The method or combination of embodiment 589, wherein the CDR-L1, CDR-L2, CDR-L3, CDR-H1, CDR-H2 and CDR-H3 sequences are those of BCMA C8.

613. The method or combination of embodiment 589, wherein the CDR-L1, CDR-L2, CDR-L3, CDR-H1, CDR-H2 and CDR-H3 sequences are those of BCMA C9.

614. The method or combination of embodiment 589, wherein the CDR-L1, CDR-L2, CDR-L3, CDR-H1, CDR-H2 and CDR-H3 sequences are those of BCMA 010.

615. The method or combination of embodiment 589, wherein the CDR-L1, CDR-L2, CDR-L3, CDR-H1, CDR-H2 and CDR-H3 sequences are those of BCMA C11.

616. The method or combination of embodiment 589, wherein the CDR-L1, CDR-L2, CDR-L3, CDR-H1, CDR-H2 and CDR-H3 sequences are those of BCMA C12.

617. The method or combination of embodiment 590, wherein the CDR-L1, CDR-L2, CDR-L3, CDR-H1, CDR-H2 and CDR-H3 sequences are those of BCMA C13.

618. The method or combination of embodiment 590, wherein the CDR-L1, CDR-L2, CDR-L3, CDR-H1, CDR-H2 and CDR-H3 sequences are those of BCMA C14.

619. The method or combination of embodiment 590, wherein the CDR-L1, CDR-L2, CDR-L3, CDR-H1, CDR-H2 and CDR-H3 sequences are those of BCMA C15.

620. The method or combination of embodiment 590, wherein the CDR-L1, CDR-L2, CDR-L3, CDR-H1, CDR-H2 and CDR-H3 sequences are those of BCMA C16.

621. The method or combination of embodiment 590, wherein the CDR-L1, CDR-L2, CDR-L3, CDR-H1, CDR-H2 and CDR-H3 sequences are those of BCMA C17.

622. The method or combination of embodiment 590, wherein the CDR-L1, CDR-L2, CDR-L3, CDR-H1, CDR-H2 and CDR-H3 sequences are those of BCMA C18.

623. The method or combination of embodiment 590, wherein the CDR-L1, CDR-L2, CDR-L3, CDR-H1, CDR-H2 and CDR-H3 sequences are those of BCMA C19.

624. The method or combination of embodiment 590, wherein the CDR-L1, CDR-L2, CDR-L3, CDR-H1, CDR-H2 and CDR-H3 sequences are those of BCMA C20.

625. The method or combination of embodiment 590, wherein the CDR-L1, CDR-L2, CDR-L3, CDR-H1, CDR-H2 and CDR-H3 sequences are those of BCMA C21.

626. The method or combination of embodiment 590, wherein the CDR-L1, CDR-L2, CDR-L3, CDR-H1, CDR-H2 and CDR-H3 sequences are those of BCMA C22.

627. The method or combination of embodiment 590, wherein the CDR-L1, CDR-L2, CDR-L3, CDR-H1, CDR-H2 and CDR-H3 sequences are those of BCMA C23.

628. The method or combination of embodiment 590, wherein the CDR-L1, CDR-L2, CDR-L3, CDR-H1, CDR-H2 and CDR-H3 sequences are those of BCMA C24.

629. The method or combination of embodiment 590, wherein the CDR-L1, CDR-L2, CDR-L3, CDR-H1, CDR-H2 and CDR-H3 sequences are those of BCMA C25.

630. The method or combination of embodiment 590, wherein the CDR-L1, CDR-L2, CDR-L3, CDR-H1, CDR-H2 and CDR-H3 sequences are those of BCMA C26.

631. The method or combination of embodiment 590, wherein the CDR-L1, CDR-L2, CDR-L3, CDR-H1, CDR-H2 and CDR-H3 sequences are those of BCMA C27.

632. The method or combination of embodiment 590, wherein the CDR-L1, CDR-L2, CDR-L3, CDR-H1, CDR-H2 and CDR-H3 sequences are those of BCMA C28.

633. The method or combination of any one of embodiments 591 to 596, wherein the CDR-L1, CDR-L2, CDR-L3, CDR-H1, CDR-H2 and CDR-H3 sequences are those of AB1.

634. The method or combination of any one of embodiments 591 to 596, wherein the CDR-L1, CDR-L2, CDR-L3, CDR-H1, CDR-H2 and CDR-H3 sequences are those of AB2.

635. The method or combination of any one of embodiments 591 to 596, wherein the CDR-L1, CDR-L2, CDR-L3, CDR-H1, CDR-H2 and CDR-H3 sequences are those of R1F2.

636. The method or combination of any one of embodiments 591 to 596, wherein the CDR-L1, CDR-L2, CDR-L3, CDR-H1, CDR-H2 and CDR-H3 sequences are those of PALF03.

637. The method or combination of any one of embodiments 591 to 596, wherein the CDR-L1, CDR-L2, CDR-L3, CDR-H1, CDR-H2 and CDR-H3 sequences are those of PALF04.

638. The method or combination of any one of embodiments 591 to 596, wherein the CDR-L1, CDR-L2, CDR-L3, CDR-H1, CDR-H2 and CDR-H3 sequences are those of PALF05.

639. The method or combination of any one of embodiments 591 to 596, wherein the CDR-L1, CDR-L2, CDR-L3, CDR-H1, CDR-H2 and CDR-H3 sequences are those of PALF06.

640. The method or combination of any one of embodiments 591 to 596, wherein the CDR-L1, CDR-L2, CDR-L3, CDR-H1, CDR-H2 and CDR-H3 sequences are those of PALF07.

641. The method or combination of any one of embodiments 591 to 596, wherein the CDR-L1, CDR-L2, CDR-L3, CDR-H1, CDR-H2 and CDR-H3 sequences are those of PALF08.

642. The method or combination of any one of embodiments 591 to 596, wherein the CDR-L1, CDR-L2, CDR-L3, CDR-H1, CDR-H2 and CDR-H3 sequences are those of PALF09.

643. The method or combination of any one of embodiments 591 to 596, wherein the CDR-L1, CDR-L2, CDR-L3, CDR-H1, CDR-H2 and CDR-H3 sequences are those of PALF12.

644. The method or combination of any one of embodiments 591 to 596, wherein the CDR-L1, CDR-L2, CDR-L3, CDR-H1, CDR-H2 and CDR-H3 sequences are those of PALF13.

645. The method or combination of any one of embodiments 591 to 596, wherein the CDR-L1, CDR-L2, CDR-L3, CDR-H1, CDR-H2 and CDR-H3 sequences are those of PALF14.

646. The method or combination of any one of embodiments 591 to 596, wherein the CDR-L1, CDR-L2, CDR-L3, CDR-H1, CDR-H2 and CDR-H3 sequences are those of PALF15.

647. The method or combination of any one of embodiments 591 to 596, wherein the CDR-L1, CDR-L2, CDR-L3, CDR-H1, CDR-H2 and CDR-H3 sequences are those of PALF16.

648. The method or combination of any one of embodiments 591 to 596, wherein the CDR-L1, CDR-L2, CDR-L3, CDR-H1, CDR-H2 and CDR-H3 sequences are those of PALF17.

649. The method or combination of any one of embodiments 591 to 596, wherein the CDR-L1, CDR-L2, CDR-L3, CDR-H1, CDR-H2 and CDR-H3 sequences are those of PALF18.

650. The method or combination of any one of embodiments 591 to 596, wherein the CDR-L1, CDR-L2, CDR-L3, CDR-H1, CDR-H2 and CDR-H3 sequences are those of PALF19.

651. The method or combination of any one of embodiments 591 to 596, wherein the CDR-L1, CDR-L2, CDR-L3, CDR-H1, CDR-H2 and CDR-H3 sequences are those of PALF20.

652. The method or combination of any one of embodiments 597 to 604, wherein the CDR-L1, CDR-L2, CDR-L3, CDR-H1, CDR-H2 and CDR-H3 sequences are those of AB3.

653. The method or combination of any one of embodiments 597 to 604, wherein the CDR-L1, CDR-L2, CDR-L3, CDR-H1, CDR-H2 and CDR-H3 sequences are those of P1-61.

654. The method or combination of any one of embodiments 597 to 604, wherein the CDR-L1, CDR-L2, CDR-L3, CDR-H1, CDR-H2 and CDR-H3 sequences are those of H2/L2-22.

655. The method or combination of any one of embodiments 597 to 604, wherein the CDR-L1, CDR-L2, CDR-L3, CDR-H1, CDR-H2 and CDR-H3 sequences are those of H2/L2-88.

656. The method or combination of any one of embodiments 597 to 604, wherein the CDR-L1, CDR-L2, CDR-L3, CDR-H1, CDR-H2 and CDR-H3 sequences are those of H2/L2-36.

657. The method or combination of any one of embodiments 597 to 604, wherein the CDR-L1, CDR-L2, CDR-L3, CDR-H1, CDR-H2 and CDR-H3 sequences are those of H2/L2-34.

658. The method or combination of any one of embodiments 597 to 604, wherein the CDR-L1, CDR-L2, CDR-L3, CDR-H1, CDR-H2 and CDR-H3 sequences are those of H2/L2-68.

659. The method or combination of any one of embodiments 597 to 604, wherein the CDR-L1, CDR-L2, CDR-L3, CDR-H1, CDR-H2 and CDR-H3 sequences are those of H2/L2-18.

660. The method or combination of any one of embodiments 597 to 604, wherein the CDR-L1, CDR-L2, CDR-L3, CDR-H1, CDR-H2 and CDR-H3 sequences are those of H2/L2-47.

661. The method or combination of any one of embodiments 597 to 604, wherein the CDR-L1, CDR-L2, CDR-L3, CDR-H1, CDR-H2 and CDR-H3 sequences are those of H2/L2-20.

662. The method or combination of any one of embodiments 597 to 604, wherein the CDR-L1, CDR-L2, CDR-L3, CDR-H1, CDR-H2 and CDR-H3 sequences are those of H2/L2-80.

663. The method or combination of any one of embodiments 597 to 604, wherein the CDR-L1, CDR-L2, CDR-L3, CDR-H1, CDR-H2 and CDR-H3 sequences are those of H2/L2-83.

664. The method or combination of any one of embodiments 597 to 604, wherein the CDR-L1, CDR-L2, CDR-L3, CDR-H1, CDR-H2 and CDR-H3 sequences are those of H3-1.

665. The method or combination of any one of embodiments 597 to 604, wherein the CDR-L1, CDR-L2, CDR-L3, CDR-H1, CDR-H2 and CDR-H3 sequences are those of H3-2.

666. The method or combination of any one of embodiments 597 to 604, wherein the CDR-L1, CDR-L2, CDR-L3, CDR-H1, CDR-H2 and CDR-H3 sequences are those of H3-3.

667. The method or combination of any one of embodiments 597 to 604, wherein the CDR-L1, CDR-L2, CDR-L3, CDR-H1, CDR-H2 and CDR-H3 sequences are those of H3-4.

668. The method or combination of any one of embodiments 597 to 604, wherein the CDR-L1, CDR-L2, CDR-L3, CDR-H1, CDR-H2 and CDR-H3 sequences are those of H3-5.

669. The method or combination of any one of embodiments 597 to 604, wherein the CDR-L1, CDR-L2, CDR-L3, CDR-H1, CDR-H2 and CDR-H3 sequences are those of H3-6.

670. The method or combination of any one of embodiments 597 to 604, wherein the CDR-L1, CDR-L2, CDR-L3, CDR-H1, CDR-H2 and CDR-H3 sequences are those of H3-7.

671. The method or combination of any one of embodiments 597 to 604, wherein the CDR-L1, CDR-L2, CDR-L3, CDR-H1, CDR-H2 and CDR-H3 sequences are those of H3-8.

672. The method or combination of any one of embodiments 597 to 604, wherein the CDR-L1, CDR-L2, CDR-L3, CDR-H1, CDR-H2 and CDR-H3 sequences are those of H3-9.

673. The method or combination of any one of embodiments 597 to 604, wherein the CDR-L1, CDR-L2, CDR-L3, CDR-H1, CDR-H2 and CDR-H3 sequences are those of H3-10.

674. The method or combination of any one of embodiments 597 to 604, wherein the CDR-L1, CDR-L2, CDR-L3, CDR-H1, CDR-H2 and CDR-H3 sequences are those of H3-11.

675. The method or combination of any one of embodiments 597 to 604, wherein the CDR-L1, CDR-L2, CDR-L3, CDR-H1, CDR-H2 and CDR-H3 sequences are those of H3-12.

676. The method or combination of any one of embodiments 597 to 604, wherein the CDR-L1, CDR-L2, CDR-L3, CDR-H1, CDR-H2 and CDR-H3 sequences are those of H3-13.

677. The method or combination of any one of embodiments 597 to 604, wherein the CDR-L1, CDR-L2, CDR-L3, CDR-H1, CDR-H2 and CDR-H3 sequences are those of H3-14.

678. The method or combination of any one of embodiments 597 to 604, wherein the CDR-L1, CDR-L2, CDR-L3, CDR-H1, CDR-H2 and CDR-H3 sequences are those of H3-15.

679. The method or combination of embodiment 587 or embodiment 588, wherein when ABM2 and/or ABM5 binds specifically to BCMA, ABM2 or ABM5 comprises a light chain variable sequence set forth in Table 15O-1 and the corresponding heavy chain variable sequence set forth in Table 15O-2.

680. The method or combination of embodiment 679, wherein the light chain variable sequence and the corresponding heavy chain variable sequence are those of AB1.

681. The method or combination of embodiment 679, wherein the light chain variable sequence and the corresponding heavy chain variable sequence are those of AB2.

682. The method or combination of embodiment 679, wherein the light chain variable sequence and the corresponding heavy chain variable sequence are those of AB3.

683. The method or combination of embodiment 679, wherein the light chain variable sequence and the corresponding heavy chain variable sequence are those of R1F2.

684. The method or combination of embodiment 679, wherein the light chain variable sequence and the corresponding heavy chain variable sequence are those of PALF03.

685. The method or combination of embodiment 679, wherein the light chain variable sequence and the corresponding heavy chain variable sequence are those of PALF04.

686. The method or combination of embodiment 679, wherein the light chain variable sequence and the corresponding heavy chain variable sequence are those of PALF05.

687. The method or combination of embodiment 679, wherein the light chain variable sequence and the corresponding heavy chain variable sequence are those of PALF06.

688. The method or combination of embodiment 679, wherein the light chain variable sequence and the corresponding heavy chain variable sequence are those of PALF07.

689. The method or combination of embodiment 679, wherein the light chain variable sequence and the corresponding heavy chain variable sequence are those of PALF08.

690. The method or combination of embodiment 679, wherein the light chain variable sequence and the corresponding heavy chain variable sequence are those of PALF09.

691. The method or combination of embodiment 679, wherein the light chain variable sequence and the corresponding heavy chain variable sequence are those of PALF12.

692. The method or combination of embodiment 679, wherein the light chain variable sequence and the corresponding heavy chain variable sequence are those of PALF13.

693. The method or combination of embodiment 679, wherein the light chain variable sequence and the corresponding heavy chain variable sequence are those of PALF14.

694. The method or combination of embodiment 679, wherein the light chain variable sequence and the corresponding heavy chain variable sequence are those of PALF15.

695. The method or combination of embodiment 679, wherein the light chain variable sequence and the corresponding heavy chain variable sequence are those of PALF16.

696. The method or combination of embodiment 679, wherein the light chain variable sequence and the corresponding heavy chain variable sequence are those of PALF17.

697. The method or combination of embodiment 679, wherein the light chain variable sequence and the corresponding heavy chain variable sequence are those of PALF18.

698. The method or combination of embodiment 679, wherein the light chain variable sequence and the corresponding heavy chain variable sequence are those of PALF19.

699. The method or combination of embodiment 679, wherein the light chain variable sequence and the corresponding heavy chain variable sequence are those of PALF20.

700. The method or combination of embodiment 679, wherein the light chain variable sequence and the corresponding heavy chain variable sequence are those of PI-61.

701. The method or combination of embodiment 679, wherein the light chain variable sequence and the corresponding heavy chain variable sequence are those of H2/L2-88.

702. The method or combination of embodiment 679, wherein the light chain variable sequence and the corresponding heavy chain variable sequence are those of H2/L2-36.

703. The method or combination of embodiment 679, wherein the light chain variable sequence and the corresponding heavy chain variable sequence are those of H2/L2-34.

704. The method or combination of embodiment 679, wherein the light chain variable sequence and the corresponding heavy chain variable sequence are those of H2/L2-68.

705. The method or combination of embodiment 679, wherein the light chain variable sequence and the corresponding heavy chain variable sequence are those of H2/L2-18.

706. The method or combination of embodiment 679, wherein the light chain variable sequence and the corresponding heavy chain variable sequence are those of H2/L2-47.

707. The method or combination of embodiment 679, wherein the light chain variable sequence and the corresponding heavy chain variable sequence are those of H2/L2-20.

708. The method or combination of embodiment 679, wherein the light chain variable sequence and the corresponding heavy chain variable sequence are those of H2/L2-80.

709. The method or combination of embodiment 679, wherein the light chain variable sequence and the corresponding heavy chain variable sequence are those of H2/L2-83.

710. The method or combination of embodiment 679, wherein the light chain variable sequence and the corresponding heavy chain variable sequence are those of H3-1.

711. The method or combination of embodiment 679, wherein the light chain variable sequence and the corresponding heavy chain variable sequence are those of H3-2.

712. The method or combination of embodiment 679, wherein the light chain variable sequence and the corresponding heavy chain variable sequence are those of H3-3.

713. The method or combination of embodiment 679, wherein the light chain variable sequence and the corresponding heavy chain variable sequence are those of H3-4.

714. The method or combination of embodiment 679, wherein the light chain variable sequence and the corresponding heavy chain variable sequence are those of H3-5.

715. The method or combination of embodiment 679, wherein the light chain variable sequence and the corresponding heavy chain variable sequence are those of H3-6.

716. The method or combination of embodiment 679, wherein the light chain variable sequence and the corresponding heavy chain variable sequence are those of H3-7.

717. The method or combination of embodiment 679, wherein the light chain variable sequence and the corresponding heavy chain variable sequence are those of H3-8.

718. The method or combination of embodiment 679, wherein the light chain variable sequence and the corresponding heavy chain variable sequence are those of H3-9.

719. The method or combination of embodiment 679, wherein the light chain variable sequence and the corresponding heavy chain variable sequence are those of H3-10.

720. The method or combination of embodiment 679, wherein the light chain variable sequence and the corresponding heavy chain variable sequence are those of H3-11.

721. The method or combination of embodiment 679, wherein the light chain variable sequence and the corresponding heavy chain variable sequence are those of H3-12.

722. The method or combination of embodiment 679, wherein the light chain variable sequence and the corresponding heavy chain variable sequence are those of H3-13.

723. The method or combination of embodiment 679, wherein the light chain variable sequence and the corresponding heavy chain variable sequence are those of H3-14.

724. The method or combination of embodiment 679, wherein the light chain variable sequence and the corresponding heavy chain variable sequence are those of H3-15.

725. The method or combination of any one of embodiments 1 to 586, wherein when ABM2 and/or ABM5 binds specifically to BCMA, ABM2 or ABM5 comprises the CDR sequences of one of BCMA-1 to BMCA-40.

726. The method or combination of embodiment 725, wherein the CDR sequences are the CDR sequences of BCMA-1.

727. The method or combination of embodiment 725, wherein the CDR sequences are the CDR sequences of BCMA-2.

728. The method or combination of embodiment 725, wherein the CDR sequences are the CDR sequences of BCMA-3.

729. The method or combination of embodiment 725, wherein the CDR sequences are the CDR sequences of BCMA-4.

730. The method or combination of embodiment 725, wherein the CDR sequences are the CDR sequences of BCMA-5.

731. The method or combination of embodiment 725, wherein the CDR sequences are the CDR sequences of BCMA-6.

732. The method or combination of embodiment 725 wherein the CDR sequences are the CDR sequences of BCMA-7.

733. The method or combination of embodiment 725, wherein the CDR sequences are the CDR sequences of BCMA-8.

734. The method or combination of embodiment 725, wherein the CDR sequences are the CDR sequences of BCMA-9.

735. The method or combination of embodiment 725, wherein the CDR sequences are the CDR sequences of BCMA-10.

736. The method or combination of embodiment 725, wherein the CDR sequences are the CDR sequences of BCMA-11.

737. The method or combination of embodiment 725, wherein the CDR sequences are the CDR sequences of BCMA-12.

738. The method or combination of embodiment 725, wherein the CDR sequences are the CDR sequences of BCMA-13.

739. The method or combination of embodiment 725, wherein the CDR sequences are the CDR sequences of BCMA-14.

740. The method or combination of embodiment 725, wherein the CDR sequences are the CDR sequences of BCMA-15.

741. The method or combination of embodiment 725, wherein the CDR sequences are the CDR sequences of BCMA-16.

742. The method or combination of embodiment 725, wherein the CDR sequences are the CDR sequences of BCMA-17.

743. The method or combination of embodiment 725, wherein the CDR sequences are the CDR sequences of BCMA-18.

744. The method or combination of embodiment 725, wherein the CDR sequences are the CDR sequences of BCMA-19.

745. The method or combination of embodiment 725, wherein the CDR sequences are the CDR sequences of BCMA-20.

746. The method or combination of embodiment 725, wherein the CDR sequences are the CDR sequences of BCMA-21.

747. The method or combination of embodiment 725, wherein the CDR sequences are the CDR sequences of BCMA-22.

748. The method or combination of embodiment 725, wherein the CDR sequences are the CDR sequences of BCMA-23.

749. The method or combination of embodiment 725, wherein the CDR sequences are the CDR sequences of BCMA-24.

750. The method or combination of embodiment 725, wherein the CDR sequences are the CDR sequences of BCMA-25.

751. The method or combination of embodiment 725, wherein the CDR sequences are the CDR sequences of BCMA-26.

752. The method or combination of embodiment 725, wherein the CDR sequences are the CDR sequences of BCMA-27.

753. The method or combination of embodiment 725, wherein the CDR sequences are the CDR sequences of BCMA-28.

754. The method or combination of embodiment 725, wherein the CDR sequences are the CDR sequences of BCMA-29.

755. The method or combination of embodiment 725, wherein the CDR sequences are the CDR sequences of BCMA-30.

756. The method or combination of embodiment 725, wherein the CDR sequences are the CDR sequences of BCMA-31.

757. The method or combination of embodiment 725, wherein the CDR sequences are the CDR sequences of BCMA-32.

758. The method or combination of embodiment 725, wherein the CDR sequences are the CDR sequences of BCMA-33.

759. The method or combination of embodiment 725, wherein the CDR sequences are the CDR sequences of BCMA-34.

760. The method or combination of embodiment 725, wherein the CDR sequences are the CDR sequences of BCMA-35.

761. The method or combination of embodiment 725, wherein the CDR sequences are the CDR sequences of BCMA-36.

762. The method or combination of embodiment 725, wherein the CDR sequences are the CDR sequences of BCMA-37.

763. The method or combination of embodiment 725, wherein the CDR sequences are the CDR sequences of BCMA-38.

764. The method or combination of embodiment 725, wherein the CDR sequences are the CDR sequences of BCMA-39.

765. The method or combination of embodiment 725, wherein the CDR sequences are the CDR sequences of BCMA-40.

766. The method or combination of any one of embodiments 725 to 765, wherein the CDRs are defined by Kabat numbering, as set forth in Table 14B and Table 14E.

767. The method or combination of any one of embodiments 725 to 765, wherein the CDRs are defined by Chothia numbering, as set forth in Table 14C and Table 14F.

768. The method or combination of any one of embodiments 725 to 765, wherein the CDRs are defined by a combination of Kabat and Chothia numbering, as set forth in Table 14D and Table 14G.

769. The method or combination of any one of embodiments 315 to 586, wherein when ABM2 and/or ABM5 binds specifically to BCMA, ABM2 or ABM5 comprises the heavy and light chain variable sequences of BCMA-1, as set forth in Table 14A.

770. The method or combination of any one of embodiments 315 to 586, wherein when ABM2 and/or ABM5 binds specifically to BCMA, ABM2 or ABM5 comprises the heavy and light chain variable sequences of BCMA-2, as set forth in Table 14A.

771. The method or combination of any one of embodiments 315 to 586, wherein when ABM2 and/or ABM5 binds specifically to BCMA, ABM2 or ABM5 comprises the heavy and light chain variable sequences of BCMA-3, as set forth in Table 14A.

772. The method or combination of any one of embodiments 315 to 586, wherein when ABM2 and/or ABM5 binds specifically to BCMA, ABM2 or ABM5 comprises the heavy and light chain variable sequences of BCMA-4, as set forth in Table 14A.

773. The method or combination of any one of embodiments 315 to 586, wherein when ABM2 and/or ABM5 binds specifically to BCMA, ABM2 or ABM5 comprises the heavy and light chain variable sequences of BCMA-5, as set forth in Table 14A.

774. The method or combination of any one of embodiments 315 to 586, wherein when ABM2 and/or ABM5 binds specifically to BCMA, ABM2 or ABM5 comprises the heavy and light chain variable sequences of BCMA-6, as set forth in Table 14A.

775. The method or combination of any one of embodiments 315 to 586, wherein when ABM2 and/or ABM5 binds specifically to BCMA, ABM2 or ABM5 comprises the heavy and light chain variable sequences of BCMA-7, as set forth in Table 14A.

776. The method or combination of any one of embodiments 315 to 586, wherein when ABM2 and/or ABM5 binds specifically to BCMA, ABM2 or ABM5 comprises the heavy and light chain variable sequences of BCMA-8, as set forth in Table 14A.

777. The method or combination of any one of embodiments 315 to 586, wherein when ABM2 and/or ABM5 binds specifically to BCMA, ABM2 or ABM5 comprises the heavy and light chain variable sequences of BCMA-9, as set forth in Table 14A.

778. The method or combination of any one of embodiments 315 to 586, wherein when ABM2 and/or ABM5 binds specifically to BCMA, ABM2 or ABM5 comprises the heavy and light chain variable sequences of BCMA-10, as set forth in Table 14A.

779. The method or combination of any one of embodiments 315 to 586, wherein when ABM2 and/or ABM5 binds specifically to BCMA, ABM2 or ABM5 comprises the heavy and light chain variable sequences of BCMA-11, as set forth in Table 14A.

780. The method or combination of any one of embodiments 315 to 586, wherein when ABM2 and/or ABM5 binds specifically to BCMA, ABM2 or ABM5 comprises the heavy and light chain variable sequences of BCMA-12, as set forth in Table 14A.

781. The method or combination of any one of embodiments 315 to 586, wherein when ABM2 and/or ABM5 binds specifically to BCMA, ABM2 or ABM5 comprises the heavy and light chain variable sequences of BCMA-13, as set forth in Table 14A.

782. The method or combination of any one of embodiments 315 to 586, wherein when ABM2 and/or ABM5 binds specifically to BCMA, ABM2 or ABM5 comprises the heavy and light chain variable sequences of BCMA-14, as set forth in Table 14A.

783. The method or combination of any one of embodiments 315 to 586, wherein when ABM2 and/or ABM5 binds specifically to BCMA, ABM2 or ABM5 comprises the heavy and light chain variable sequences of BCMA-15, as set forth in Table 14A.

784. The method or combination of any one of embodiments 315 to 586, wherein when ABM2 and/or ABM5 binds specifically to BCMA, ABM2 or ABM5 comprises the heavy and light chain variable sequences of BCMA-16, as set forth in Table 14A.

785. The method or combination of any one of embodiments 315 to 586, wherein when ABM2 and/or ABM5 binds specifically to BCMA, ABM2 or ABM5 comprises the heavy and light chain variable sequences of BCMA-17, as set forth in Table 14A.

786. The method or combination of any one of embodiments 315 to 586, wherein when ABM2 and/or ABM5 binds specifically to BCMA, ABM2 or ABM5 comprises the heavy and light chain variable sequences of BCMA-18, as set forth in Table 14A.

787. The method or combination of any one of embodiments 315 to 586, wherein when ABM2 and/or ABM5 binds specifically to BCMA, ABM2 or ABM5 comprises the heavy and light chain variable sequences of BCMA-19, as set forth in Table 14A.

788. The method or combination of any one of embodiments 315 to 586, wherein when ABM2 and/or ABM5 binds specifically to BCMA, ABM2 or ABM5 comprises the heavy and light chain variable sequences of BCMA-20, as set forth in Table 14A.

789. The method or combination of any one of embodiments 315 to 586, wherein when ABM2 and/or ABM5 binds specifically to BCMA, ABM2 or ABM5 comprises the heavy and light chain variable sequences of BCMA-21, as set forth in Table 14A.

790. The method or combination of any one of embodiments 315 to 586, wherein when ABM2 and/or ABM5 binds specifically to BCMA, ABM2 or ABM5 comprises the heavy and light chain variable sequences of BCMA-22, as set forth in Table 14A.

791. The method or combination of any one of embodiments 315 to 586, wherein when ABM2 and/or ABM5 binds specifically to BCMA, ABM2 or ABM5 comprises the heavy and light chain variable sequences of BCMA-23, as set forth in Table 14A.

792. The method or combination of any one of embodiments 315 to 586, wherein when ABM2 and/or ABM5 binds specifically to BCMA, ABM2 or ABM5 comprises the heavy and light chain variable sequences of BCMA-24, as set forth in Table 14A.

793. The method or combination of any one of embodiments 315 to 586, wherein when ABM2 and/or ABM5 binds specifically to BCMA, ABM2 or ABM5 comprises the heavy and light chain variable sequences of BCMA-25, as set forth in Table 14A.

794. The method or combination of any one of embodiments 315 to 586, wherein when ABM2 and/or ABM5 binds specifically to BCMA, ABM2 or ABM5 comprises the heavy and light chain variable sequences of BCMA-26, as set forth in Table 14A.

795. The method or combination of any one of embodiments 315 to 586, wherein when ABM2 and/or ABM5 binds specifically to BCMA, ABM2 or ABM5 comprises the heavy and light chain variable sequences of BCMA-27, as set forth in Table 14A.

796. The method or combination of any one of embodiments 315 to 586, wherein when ABM2 and/or ABM5 binds specifically to BCMA, ABM2 or ABM5 comprises the heavy and light chain variable sequences of BCMA-28, as set forth in Table 14A.

797. The method or combination of any one of embodiments 315 to 586, wherein when ABM2 and/or ABM5 binds specifically to BCMA, ABM2 or ABM5 comprises the heavy and light chain variable sequences of BCMA-29, as set forth in Table 14A.

798. The method or combination of any one of embodiments 315 to 586, wherein when ABM2 and/or ABM5 binds specifically to BCMA, ABM2 or ABM5 comprises the heavy and light chain variable sequences of BCMA-30, as set forth in Table 14A.

799. The method or combination of any one of embodiments 315 to 586, wherein when ABM2 and/or ABM5 binds specifically to BCMA, ABM2 or ABM5 comprises the heavy and light chain variable sequences of BCMA-31, as set forth in Table 14A.

800. The method or combination of any one of embodiments 315 to 586, wherein when ABM2 and/or ABM5 binds specifically to BCMA, ABM2 or ABM5 comprises the heavy and light chain variable sequences of BCMA-32, as set forth in Table 14A.

801. The method or combination of any one of embodiments 315 to 586, wherein when ABM2 and/or ABM5 binds specifically to BCMA, ABM2 or ABM5 comprises the heavy and light chain variable sequences of BCMA-33, as set forth in Table 14A.

802. The method or combination of any one of embodiments 315 to 586, wherein when ABM2 and/or ABM5 binds specifically to BCMA, ABM2 or ABM5 comprises the heavy and light chain variable sequences of BCMA-34, as set forth in Table 14A.

803. The method or combination of any one of embodiments 315 to 586, wherein when ABM2 and/or ABM5 binds specifically to BCMA, ABM2 or ABM5 comprises the heavy and light chain variable sequences of BCMA-35, as set forth in Table 14A.

804. The method or combination of any one of embodiments 315 to 586, wherein when ABM2 and/or ABM5 binds specifically to BCMA, ABM2 or ABM5 comprises the heavy and light chain variable sequences of BCMA-36, as set forth in Table 14A.

805. The method or combination of any one of embodiments 315 to 586, wherein when ABM2 and/or ABM5 binds specifically to BCMA, ABM2 or ABM5 comprises the heavy and light chain variable sequences of BCMA-37, as set forth in Table 14A.

806. The method or combination of any one of embodiments 315 to 586, wherein when ABM2 and/or ABM5 binds specifically to BCMA, ABM2 or ABM5 comprises the heavy and light chain variable sequences of BCMA-38, as set forth in Table 14A.

807. The method or combination of any one of embodiments 315 to 586, wherein when ABM2 and/or ABM5 binds specifically to BCMA, ABM2 or ABM5 comprises the heavy and light chain variable sequences of BCMA-39, as set forth in Table 14A.

808. The method or combination of any one of embodiments 315 to 586, wherein when ABM2 and/or ABM5 binds specifically to BCMA, ABM2 or ABM5 comprises the heavy and light chain variable sequences of BCMA-40, as set forth in Table 14A.

809. The method or combination of any one of embodiments 315 to 551, wherein when ABM2 and/or ABM5 binds specifically to CD20, ABM2 or ABM5 comprises the CDR-H1, CDR-H2, CDR-H3, CDR-L1, CDR-L2, and CDR-L3 sequence of CD20_1 as defined by Kabat and set forth in Table 18.

810. The method or combination of any one of embodiments 315 to 551, wherein when ABM2 and/or ABM5 binds specifically to CD20, ABM2 or ABM5 comprises the CDR-H1, CDR-H2, CDR-H3, CDR-L1, CDR-L2, and CDR-L3 sequence of CD20_1 as defined by Chothia and set forth in Table 18.

811. The method or combination of any one of embodiments 315 to 551, wherein when ABM2 and/or ABM5 binds specifically to CD20, ABM2 or ABM5 comprises the CDR-H1, CDR-H2, CDR-H3, CDR-L1, CDR-L2, and CDR-L3 sequence of CD20_1 as defined by IMGT and set forth in Table 18.

812. The method or combination of any one of embodiments 315 to 551, wherein when ABM2 and/or ABM5 binds specifically to CD20, ABM2 or ABM5 comprises the CDR-H1, CDR-H2, CDR-H3, CDR-L1, CDR-L2, and CDR-L3 sequence of CD20_1 as defined by the combination of Kabat and Chothia and set forth in Table 18.

813. The method or combination of any one of embodiments 315 to 551, wherein when ABM2 and/or ABM5 binds specifically to CD20, ABM2 or ABM5 comprises the heavy chain and/or light chain variable sequences of CD20_1 as set forth in Table 18.

814. The method or combination of any one of embodiments 315 to 551, wherein when ABM2 and/or ABM5 binds specifically to CD22, ABM2 or ABM5 comprises the CDR-H1, CDR-H2, CDR-H3, CDR-L1, CDR-L2, and CDR-L3 sequence of CD22_HA22 as defined by Kabat and set forth in Table 19.

815. The method or combination of any one of embodiments 315 to 551, wherein when ABM2 and/or ABM5 binds specifically to CD22, ABM2 or ABM5 comprises the CDR-H1, CDR-H2, CDR-H3, CDR-L1, CDR-L2, and CDR-L3 sequence of CD22_HA22 as defined by Chothia and set forth in Table 19.

816. The method or combination of any one of embodiments 315 to 551, wherein when ABM2 and/or ABM5 binds specifically to CD22, ABM2 or ABM5 comprises the CDR-H1, CDR-H2, CDR-H3, CDR-L1, CDR-L2, and CDR-L3 sequence of CD22_HA22 as defined by IMGT and set forth in Table 19.

817. The method or combination of any one of embodiments 315 to 551, wherein when ABM2 and/or ABM5 binds specifically to CD22, ABM2 or ABM5 comprises the CDR-H1, CDR-H2, CDR-H3, CDR-L1, CDR-L2, and CDR-L3 sequence of CD22_HA22 as defined by the combination of Kabat and Chothia and set forth in Table 19.

818. The method or combination of any one of embodiments 315 to 551, wherein when ABM2 and/or ABM5 binds specifically to CD22, ABM2 or ABM5 comprises the heavy chain and/or light chain variable sequences of CD22_HA22 as set forth in Table 19.

819. The method or combination of any one of embodiments 315 to 551, wherein when ABM2 and/or ABM5 binds specifically to CD22, ABM2 or ABM5 comprises the CDR-H1, CDR-H2, CDR-H3, CDR-L1, CDR-L2, and CDR-L3 sequence of CD22_ m971 as defined by Kabat and set forth in Table 19.

820. The method or combination of any one of embodiments 315 to 551, wherein when ABM2 and/or ABM5 binds specifically to CD22, ABM2 or ABM5 comprises the CDR-H1, CDR-H2, CDR-H3, CDR-L1, CDR-L2, and CDR-L3 sequence of CD22_m971 as defined by Chothia and set forth in Table 19.

821. The method or combination of any one of embodiments 315 to 551, wherein when ABM2 and/or ABM5 binds specifically to CD22, ABM2 or ABM5 comprises the CDR-H1, CDR-H2, CDR-H3, CDR-L1, CDR-L2, and CDR-L3 sequence of CD22_ m971 as defined by IMGT and set forth in Table 19.

822. The method or combination of any one of embodiments 315 to 551, wherein when ABM2 and/or ABM5 binds specifically to CD22, ABM2 or ABM5 comprises the CDR-H1, CDR-H2, CDR-H3, CDR-L1, CDR-L2, and CDR-L3 sequence of CD22_ m971 as defined by the combination of Kabat and Chothia and set forth in Table 19.

823. The method or combination of any one of embodiments 315 to 551, wherein when ABM2 and/or ABM5 binds specifically to CD22, ABM2 or ABM5 comprises the heavy chain and/or light chain variable sequences of CD22_m971 as set forth in Table 19.

824. The method or combination of any one of embodiments 315 to 551, wherein when ABM2 and/or ABM5 binds specifically to CD22, ABM2 or ABM5 comprises the CDR-H1, CDR-H2, CDR-H3, CDR-L1, CDR-L2, and CDR-L3 sequence of CD22_65 as defined by Kabat and set forth in Table 19.

825. The method or combination of any one of embodiments 315 to 551, wherein when ABM2 and/or ABM5 binds specifically to CD22, ABM2 or ABM5 comprises the CDR-H1, CDR-H2, CDR-H3, CDR-L1, CDR-L2, and CDR-L3 sequence of CD22_65 as defined by Chothia and set forth in Table 19.

826. The method or combination of any one of embodiments 315 to 551, wherein when ABM2 and/or ABM5 binds specifically to CD22, ABM2 or ABM5 comprises the CDR-H1, CDR-H2, CDR-H3, CDR-L1, CDR-L2, and CDR-L3 sequence of CD22_ 65 as defined by IMGT and set forth in Table 19.

827. The method or combination of any one of embodiments 315 to 551, wherein when ABM2 and/or ABM5 binds specifically to CD22, ABM2 or ABM5 comprises the CDR-H1, CDR-H2, CDR-H3, CDR-L1, CDR-L2, and CDR-L3 sequence of CD22_ 65 as defined by the combination of Kabat and Chothia and set forth in Table 19.

828. The method or combination of any one of embodiments 315 to 551, wherein when ABM2 and/or ABM5 binds specifically to CD22, ABM2 or ABM5 comprises the heavy chain and/or light chain variable sequences of CD22_65 as set forth in Table 19.

829. The method or combination of any one of embodiments 315 to 551, wherein when ABM2 and/or ABM5 binds specifically to MSLN, ABM2 or ABM5 comprises the CDR-H1, CDR-H2, CDR-H3, CDR-L1, CDR-L2, and CDR-L3 sequence of MSLN_SS1 as defined by Kabat and set forth in Table 20.

830. The method or combination of any one of embodiments 315 to 551, wherein when ABM2 and/or ABM5 binds specifically to MSLN, ABM2 or ABM5 comprises the CDR-H1, CDR-H2, CDR-H3, CDR-L1, CDR-L2, and CDR-L3 sequence of MSLN_SS1 as defined by Chothia and set forth in Table 20.

831. The method or combination of any one of embodiments 315 to 551, wherein when ABM2 and/or ABM5 binds specifically to MSLN, ABM2 or ABM5 comprises the CDR-H1, CDR-H2, CDR-H3, CDR-L1, CDR-L2, and CDR-L3 sequence of MSLN_SS1 as defined by IMGT and set forth in Table 20.

832. The method or combination of any one of embodiments 315 to 551, wherein when ABM2 and/or ABM5 binds specifically to MSLN, ABM2 or ABM5 comprises the CDR-H1, CDR-H2, CDR-H3, CDR-L1, CDR-L2, and CDR-L3 sequence of MSLN_SS1 as defined by the combination of Kabat and Chothia and set forth in Table 20.

833. The method or combination of any one of embodiments 315 to 551, wherein when ABM2 and/or ABM5 binds specifically to MSLN, ABM2 or ABM5 comprises the heavy chain and/or light chain variable sequences of MSLN_SS1 as set forth in Table 20.

834. The method or combination of any one of embodiments 315 to 551, wherein when ABM2 and/or ABM5 binds specifically to MSLN, ABM2 or ABM5 comprises the CDR-H1, CDR-H2, CDR-H3, CDR-L1, CDR-L2, and CDR-L3 sequence of MSLN_M5 as defined by Kabat and set forth in Table 20.

835. The method or combination of any one of embodiments 315 to 551, wherein when ABM2 and/or ABM5 binds specifically to MSLN, ABM2 or ABM5 comprises the CDR-H1, CDR-H2, CDR-H3, CDR-L1, CDR-L2, and CDR-L3 sequence of MSLN_M5 as defined by Chothia and set forth in Table 20.

836. The method or combination of any one of embodiments 315 to 551, wherein when ABM2 and/or ABM5 binds specifically to MSLN, ABM2 or ABM5 comprises the CDR-H1, CDR-H2, CDR-H3, CDR-L1, CDR-L2, and CDR-L3 sequence of MSLN_M5 as defined by IMGT and set forth in Table 20.

837. The method or combination of any one of embodiments 315 to 551, wherein when ABM2 and/or ABM5 binds specifically to MSLN, ABM2 or ABM5 comprises the CDR-H1, CDR-H2, CDR-H3, CDR-L1, CDR-L2, and CDR-L3 sequence of MSLN_M5 as defined by the combination of Kabat and Chothia and set forth in Table 20.

838. The method or combination of any one of embodiments 315 to 551, wherein when ABM2 and/or ABM5 binds specifically to MSLN, ABM2 or ABM5 comprises the heavy chain and/or light chain variable sequences of MSLN_M5 as set forth in Table 20.

839. The method or combination of any one of embodiments 1 to 838, wherein the first MBM comprises an ABM3 and/or the second MBM comprises an ABM6.

840. The method or combination of embodiment 839, wherein the first MBM comprises an ABM3.

841. The method or combination of embodiment 840, wherein ABM3 is a non-immunoglobulin scaffold based ABM.

842. The method or combination of embodiment 841, wherein ABM3 is a Kunitz domain, an Adnexin, an Affibody, a DARPin, an Avimer, an Anticalin, a Lipocalin, a Centyrin, a Versabody, a Knottin, an Adnectin, a Pronectin, an Affitin/Nanofitin, an Affilin, an Atrimer/Tetranectin, a bicyclic peptide, a cys-knot, a Fn3 scaffold, an Obody, a Tn3, an Affimer, BD, an Adhiron, a Duocalin, an Alphabody, an Armadillo Repeat Protein, a Repebody, or a Fynomer.

843. The method or combination of embodiment 840, wherein ABM3 is an immunoglobulin scaffold based ABM.

844. The method or combination of embodiment 843, wherein ABM3 is an antibody, an antibody fragment, an scFv, a dsFv, a Fv, a Fab, an scFab, a (Fab′)2, a single domain antibody (SDAB), a VH or VL domain, or a camelid VHH domain.

845. The method or combination of embodiment 844, wherein ABM3 is an antibody or an antigen-binding domain thereof.

846. The method or combination of embodiment 844, wherein ABM3 is an scFv.

847. The method or combination of embodiment 844, wherein ABM3 is a Fab.

848. The method or combination of embodiment 847, wherein ABM3 is a Fab heterodimer.

849. The method or combination of any one of embodiments 839 to 848, wherein the second MBM comprises an ABM6.

850. The method or combination of embodiment 849, wherein ABM6 is a non-immunoglobulin scaffold based ABM.

851. The method or combination of embodiment 850, wherein ABM6 is a Kunitz domain, an Adnexin, an Affibody, a DARPin, an Avimer, an Anticalin, a Lipocalin, a Centyrin, a Versabody, a Knottin, an Adnectin, a Pronectin, an Affitin/Nanofitin, an Affilin, an Atrimer/Tetranectin, a bicyclic peptide, a cys-knot, a Fn3 scaffold, an Obody, a Tn3, an Affimer, BD, an Adhiron, a Duocalin, an Alphabody, an Armadillo Repeat Protein, a Repebody, or a Fynomer.

852. The method or combination of embodiment 849, wherein ABM6 is an immunoglobulin scaffold based ABM.

853. The method or combination of embodiment 852, wherein ABM6 is an antibody, an antibody fragment, an scFv, a dsFv, a Fv, a Fab, an scFab, a (Fab′)2, a single domain antibody (SDAB), a VH or VL domain, or a camelid VHH domain.

854. The method or combination of embodiment 853, wherein ABM6 is an antibody or an antigen-binding domain thereof.

855. The method or combination of embodiment 854, wherein ABM6 is an scFv.

856. The method or combination of embodiment 854, wherein ABM6 is a Fab.

857. The method or combination of embodiment 856, wherein ABM6 is a Fab heterodimer.

858. The method or combination of any one of embodiments 839 to 857, wherein the first MBM comprises an ABM3 and the second MBM comprises an ABM6.

859. The method or combination of embodiment 858, wherein ABM3 and ABM6 bind specifically to the same TMEA.

860. The method or combination of embodiment 859, wherein the TMEA is APRIL, FAP, BAFF, IL-1R, VEGF-A, VEGFR, CSF1R, ανβ3, or α5β1.

861. The method or combination of embodiment 860, wherein the TMEA is APRIL.

862. The method or combination of embodiment 860, wherein the TMEA is FAP.

863. The method or combination of embodiment 860, wherein the TMEA is BAFF.

864. The method or combination of embodiment 860, wherein the TMEA is IL-1R.

865. The method or combination of embodiment 860, wherein the TMEA is VEGF-A.

866. The method or combination of embodiment 860, wherein the TMEA is VEGFR.

867. The method or combination of embodiment 860, wherein the TMEA is CSF1R.

868. The method or combination of embodiment 860, wherein the TMEA is ανβ3.

869. The method or combination of embodiment 860, wherein the TMEA is α5β1.

870. The method or combination of any one of embodiments 859 to 869, wherein ABM3 and ABM6 bind specifically to different epitopes on the same TMEA.

871. The method or combination of embodiment 870, wherein the different epitopes do not overlap.

872. The method or combination of any one of embodiments 859 to 871, wherein the first MBM and second MBM are capable of specifically binding the TMEA simultaneously.

873. The method or combination of any one of embodiments 859 to 872, wherein binding of the first MBM to the TMEA reduces binding of the second MBM to the TMEA by less than 50% in a competition assay.

874. The method or combination of any one of embodiments 859 to 872, wherein binding of the first MBM to the TMEA reduces binding of the second MBM to the TMEA by less than 40% in a competition assay.

875. The method or combination of any one of embodiments 859 to 872, wherein binding of the first MBM to the TMEA reduces binding of the second MBM to the TMEA by less than 30% in a competition assay.

876. The method or combination of any one of embodiments 859 to 872, wherein binding of the first MBM to the TMEA reduces binding of the second MBM to the TMEA by less than 20% in a competition assay.

877. The method or combination of any one of embodiments 859 to 872, wherein binding of the first MBM to the TMEA reduces binding of the second MBM to the TMEA by less than 10% in a competition assay.

878. The method or combination of any one of embodiments 873 to 877, wherein the competition assay is an ELISA assay, a Biacore assay, a FACS assay.

879. The method or combination of any one of embodiments 839 to 858, wherein ABM3, when present in the first MBM, binds specifically to a first TMEA (“TMEA 1”) and ABM6, when present in the second MBM, binds specifically to a second TMEA (“TMEA 2”), and wherein when ABM3 is present in the first MBM and ABM6 is present in the second MBM, TMEA 1 and TMEA 2 are different TMEAs.

880. The method or combination of embodiment 879, wherein TMEA 1 and TMEA 2 are selected from APRIL, FAP, BAFF, IL-1R, VEGF-A, VEGFR, CSF1R, ανβ3, and α5β1.

881. The method or combination of embodiment 879 or 880, wherein TMEA 1 is APRIL.

882. The method or combination of embodiment 879 or 880, wherein TMEA 1 is FAP.

883. The method or combination of embodiment 879 or 880, wherein TMEA 1 is BAFF.

884. The method or combination of embodiment 879 or 880, wherein TMEA 1 is IL-1R.

885. The method or combination of embodiment 879 or 880, wherein TMEA 1 is VEGF-A.

886. The method or combination of embodiment 879 or 880, wherein TMEA 1 is VEGFR.

887. The method or combination of embodiment 879 or 880, wherein TMEA 1 is CSF1R.

888. The method or combination of embodiment 879 or 880, wherein TMEA 1 is ανβ3.

889. The method or combination of embodiment 879 or 880, wherein TMEA 1 is α5β1.

890. The method or combination of any one of embodiments 879 to 889 other than embodiments where TMEA 1 is APRIL, wherein TMEA 2 is APRIL.

891. The method or combination of any one of embodiments 879 to 889 other than embodiments where TMEA 1 is FAP, wherein TMEA 2 is FAP.

892. The method or combination of any one of embodiments 879 to 889 other than embodiments where TMEA 1 is BAFF, wherein TMEA 2 is BAFF.

893. The method or combination of any one of embodiments 879 to 889 other than embodiments where TMEA 1 is IL-1R, wherein TMEA 2 is IL-1R.

894. The method or combination of any one of embodiments 879 to 889 other than embodiments where TMEA 1 is VEGF-A, wherein TMEA 2 is VEGF-A.

895. The method or combination of any one of embodiments 879 to 889 other than embodiments where TMEA 1 is VEGFR, wherein TMEA 2 is VEGFR.

896. The method or combination of any one of embodiments 879 to 889 other than embodiments where TMEA 1 is CSF1R, wherein TMEA 2 is CSF1R.

897. The method or combination of any one of embodiments 879 to 889 other than embodiments where TMEA 1 is ανβ3, wherein TMEA 2 is ανβ3.

898. The method or combination of any one of embodiments 879 to 889 other than embodiments where TMEA 1 is α5β1, wherein TMEA 2 is α5β1.

899. The method or combination of any one of embodiments 879 to 898, wherein TMEA 1 and TMEA 2 are expressed on the same cell.

900. The method or combination of any one of embodiments 879 to 898, wherein TMEA 1 and TMEA2 2 are expressed on different cells.

901. The method or combination of any one of embodiments 839 to 900, wherein ABM5 and/or AMB6, when present, each independent of the other, comprises the CDR sequences of an antibody set forth in Table 21.

902. The method or combination of embodiment 901, wherein ABM5 and/or ABM6, when present, each independent of the other, comprises the heavy and light chain variable region sequences of an antibody set forth in Table 21.

903. The method or combination of any one of embodiments 1 to 902, wherein the first MBM and the second MBM in combination show an additive amount of T cell mediated apoptosis in an in vitro re-directed T cell cytotoxicity assay as compared to the first MBM alone and the second MBM alone.

904. The method or combination of any one of embodiments 1 to 903, wherein the first MBM and the second MBM in combination show an additive amount of cytokine release in an in vitro cytokine release assay as compared to the first MBM alone and the second MBM alone.

905. The method or combination of any one of embodiments 1 to 904, wherein the first MBM and the second MBM in combination show an additive amount of T cell proliferation in an in vitro T cell proliferation assay as compared to the first MBM alone and the second MBM alone.

906. The method or combination of any one of embodiments 1 to 905, wherein the first MBM and the second MBM in combination show an increased amount of T cell activation in an in vitro T cell activation assay as compared to the first MBM alone and the second MBM alone.

907. The method or combination of any one of embodiments 1 to 906, wherein when ABM2 is present, the TAA to which ABM2 specifically binds is upregulated in the proliferative disease or autoimmune disorder.

908. The method or combination of any one of embodiments 1 to 907, wherein when ABM5 is present, the TAA to which ABM5 specifically binds is upregulated in the proliferative disease or autoimmune disorder.

909. The method or combination of any one of embodiments 1 to 908, wherein the first MBM and/or second MBM comprises a first variant Fc region and a second variant Fc region that together form an Fc heterodimer.

910. The method or combination of embodiment 909, wherein the first and second variant Fc regions comprise the amino acid substitutions S364K/E357Q:L368D/K370S.

911. The method or combination of embodiments 909, wherein the first and second variant Fc regions comprise the amino acid substitutions L368D/K370S:S364K.

912. The method or combination of embodiment 909, wherein the first and second variant Fc regions comprise the amino acid substitutions L368E/K370S:S364K.

913. The method or combination of embodiment 909, wherein the first and second variant Fc regions comprise the amino acid substitutions T411T/E360E/Q362E:D401K.

914. The method or combination of embodiment 909, wherein the first and second variant Fc regions comprise the amino acid substitutions L368D 370S:S364/E357L.

915. The method or combination of embodiment 909, wherein the first and second variant Fc regions comprise the amino acid substitutions 370S:S364K/E357Q.

916. The method or combination of embodiment 909, wherein the first and second variant Fc regions comprise the amino acid substitutions of any one of the steric variants listed in FIG. 4 of WO 2014/110601 (reproduced in Table 3).

917. The method or combination of embodiment 909, wherein the first and second variant Fc regions comprise the amino acid substitutions of any one of the variants listed in FIG. 5 of WO 2014/110601 (reproduced in Table 3).

918. The method or combination of embodiment 909, wherein the first and second variant Fc regions comprise the amino acid substitutions of any one of the variants listed in FIG. 6 of WO 2014/110601 (reproduced in Table 3).

919. The method or combination of any one of embodiments 909 to 918, wherein at least one of the Fc regions comprises an ablation variant modification.

920. The method or combination of embodiment 919, wherein the ablation variant modifications are selected from Table 2.

921. The method or combination of embodiment 920, wherein the ablation variant modification comprises G236R.

922. The method or combination of embodiment 920, wherein the ablation variant modification comprises S239G.

923. The method or combination of embodiment 920, wherein the ablation variant modification comprises S239K.

924. The method or combination of embodiment 920, wherein the ablation variant modification comprises S239Q.

925. The method or combination of embodiment 920, wherein the ablation variant modification comprises S239R.

926. The method or combination of embodiment 920, wherein the ablation variant modification comprises V266D.

927. The method or combination of embodiment 920, wherein the ablation variant modification comprises S267K.

928. The method or combination of embodiment 920, wherein the ablation variant modification comprises S267R.

929. The method or combination of embodiment 920, wherein the ablation variant modification comprises H268K.

930. The method or combination of embodiment 920, wherein the ablation variant modification comprises E269R.

931. The method or combination of embodiment 920, wherein the ablation variant modification comprises 299R.

932. The method or combination of embodiment 920, wherein the ablation variant modification comprises 299K

933. The method or combination of embodiment 920, wherein the ablation variant modification comprises K322A

934. The method or combination of embodiment 920, wherein the ablation variant modification comprises A327G

935. The method or combination of embodiment 920, wherein the ablation variant modification comprises A327L

936. The method or combination of embodiment 920, wherein the ablation variant modification comprises A327N

937. The method or combination of embodiment 920, wherein the ablation variant modification comprises A327Q

938. The method or combination of embodiment 920, wherein the ablation variant modification comprises L328E

939. The method or combination of embodiment 920, wherein the ablation variant modification comprises L328R

940. The method or combination of embodiment 920, wherein the ablation variant modification comprises P329A

941. The method or combination of embodiment 920, wherein the ablation variant modification comprises P329H

942. The method or combination of embodiment 920, wherein the ablation variant modification comprises P329K

943. The method or combination of embodiment 920, wherein the ablation variant modification comprises A330L

944. The method or combination of embodiment 920, wherein the ablation variant modification comprises A330S/P331S

945. The method or combination of embodiment 920, wherein the ablation variant modification comprises I332K

946. The method or combination of embodiment 920, wherein the ablation variant modification comprises I332R

947. The method or combination of embodiment 920, wherein the ablation variant modification comprises V266D/A327Q

948. The method or combination of embodiment 920, wherein the ablation variant modification comprises V266D/P329K

949. The method or combination of embodiment 920, wherein the ablation variant modification comprises G236R/L328R

950. The method or combination of embodiment 920, wherein the ablation variant modification comprises E233P/L234V/L235A/G236del/S239K.

951. The method or combination of embodiment 920, wherein the ablation variant modification comprises E233P/L234V/L235A/G236del/S267K.

952. The method or combination of embodiment 920, wherein the ablation variant modification comprises E233P/L234V/L235A/G236del/S239K/A327G.

953. The method or combination of embodiment 920, wherein the ablation variant modification comprises E233P/L234V/L235A/G236del/S267K/A327G.

954. The method or combination of embodiment 920, wherein the ablation variant modification comprises E233P/L234V/L235A/G236del.

955. The method or combination of embodiment 920, wherein the ablation variant modification comprises S239K/S267K.

956. The method or combination of embodiment 920, wherein the ablation variant modification comprises 267K/P329K.

957. The method or combination of any one of embodiments 919 to 956, wherein the Fc region comprising the ablation variant modification is operably linked to ABM1.

958. The method or combination of any one of embodiments 919 to 956, wherein the Fc region comprising the ablation variant modification is operably linked to ABM2.

959. The method or combination of any one of embodiments 919 to 956, wherein the Fc region comprising the ablation variant modification is operably linked to ABM4.

960. The method or combination of any one of embodiments 919 to 956, wherein the Fc region comprising the ablation variant modification is operably linked to ABM5.

961. The method or combination of any one of embodiments 919 to 956, wherein both variant Fc regions comprise the ablation variant modification.

962. The method or combination of any one of embodiments 909 to 961, wherein at least one of the Fc regions further comprises pI variant substitutions.

963. The method or combination of embodiment 962 wherein the pI variant substitutions are selected from Table 3.

964. The method or combination of embodiment 963, wherein the pI variant substitutions comprise the substitutions present in pI_ISO(−).

965. The method or combination of embodiment 963, wherein the pI variant substitutions comprise the substitutions present in pI_(−)_isosteric_A.

966. The method or combination of embodiment 963, wherein the pI variant substitutions comprise the substitutions present in pI_(−)_isosteric_B.

967. The method or combination of embodiment 963, wherein the p1 variant substitutions comprise the substitutions present in PI_ISO(+RR).

968. The method or combination of embodiment 963, wherein the p1 variant substitutions comprise the substitutions present in pI_ISO(+).

969. The method or combination of embodiment 963, wherein the p1 variant substitutions comprise the substitutions present in pI_(+)_isosteric_A.

970. The method or combination of embodiment 963, wherein the p1 variant substitutions comprise the substitutions present in pI_(+)_isosteric_B.

971. The method or combination of embodiment 963, wherein the p1 variant substitutions comprise the substitutions present in pI_(+)_isosteric_E269Q/E272Q.

972. The method or combination of embodiment 963, wherein the p1 variant substitutions comprise the substitutions present in pI_(+)_isosteric_E269Q/E283Q.

973. The method or combination of embodiment 963, wherein the p1 variant substitutions comprise the substitutions present in pI_(+)_isosteric_E2720/E283Q.

974. The method or combination of embodiment 963, wherein the p1 variant substitutions comprise the substitutions present in pI_(+)_isosteric_E269Q.

975. The method or combination of any one of embodiments 909 to 974, wherein the first and/or second Fc region further comprises one or more amino acid substitution(s) selected from 434A, 434S, 428L, 308F, 259I, 428L/434S, 259I/308F, 436I/428L, 436I or V/434S, 436V/428L, 252Y, 252Y/254T/256E, 259I/308F/428L, 236A, 239D, 239E, 332E, 332D, 239D/332E, 267D, 267E, 328F, 267E/328F, 236A/332E, 239D/332E/330Y, 239D, 332E/330L, 236R, 328R, 236R/328R, 236N/267E, 243L, 298A and 299T.

976. The method or combination of any one of embodiments 909 to 974, wherein the first and/or second Fc region further comprises the amino acid substitution 434A, 434S or 434V.

977. The method or combination of embodiment 976, wherein the first and/or second Fc region further comprises the amino acid substitution 428L.

978. The method or combination of any one of embodiments 976 to 977, wherein the first and/or second Fc region further comprises the amino acid substitution 308F.

979. The method or combination of any one of embodiments 976 to 978, wherein the first and/or second Fc region further comprises the amino acid substitution 259I.

980. The method or combination of any one of embodiments 976 to 979, wherein the first and/or second Fc region further comprises the amino acid substitution 436I.

981. The method or combination of any one of embodiments 976 to 980, wherein the first and/or second Fc region further comprises the amino acid substitution 252Y.

982. The method or combination of any one of embodiments 976 to 981, wherein the first and/or second Fc region further comprises the amino acid substitution 254T.

983. The method or combination of any one of embodiments 976 to 982, wherein the first and/or second Fc region further comprises the amino acid substitution 256E.

984. The method or combination of any one of embodiments 976 to 983, wherein the first and/or second Fc region further comprises the amino acid substitution 239D or 239E.

985. The method or combination of any one of embodiments 976 to 984, wherein the first and/or second Fc region further comprises the amino acid substitution 332E or 332D.

986. The method or combination of any one of embodiments 976 to 985, wherein the first and/or second Fc region further comprises the amino acid substitution 267D or 267E.

987. The method or combination of any one of embodiments 976 to 986, wherein the first and/or second Fc region further comprises the amino acid substitution 330L.

988. The method or combination of any one of embodiments 976 to 987, wherein the first and/or second Fc region further comprises the amino acid substitution 236R or 236N.

989. The method or combination of any one of embodiments 976 to 988, wherein the first and/or second Fc region further comprises the amino acid substitution 328R.

990. The method or combination of any one of embodiments 976 to 989, wherein the first and/or second Fc region further comprises the amino acid substitution 243L.

991. The method or combination of any one of embodiments 976 to 990, wherein the first and/or second Fc region further comprises the amino acid substitution 298A.

992. The method or combination of any one of embodiments 976 to 991, wherein the first and/or second Fc region further comprises the amino acid substitution 299T.

993. The method or combination of embodiment 909, wherein:

(a) the first and second variant Fc regions comprise the amino acid substitutions S364K/E357Q:L368D/K370S;

(b) the first and/or second variant Fc regions comprises the ablation variant modifications E233P/L234V/L235A/G236del/S267K, and

(c) the first and/or second variant Fc regions comprises the pI variant substitutions N208D/Q295E/N384D/Q418E/N421D (pI_(−)_isosteric_A).

994. The method or combination of embodiment 993, wherein the first variant Fc region comprises the ablation variant modifications E233P/L234V/L235A/G236del/S267K.

995. The method or combination of any one of embodiments 993 to 994, wherein the second variant Fc region comprises the ablation variant modifications E233P/L234V/L235A/G236del/S267K.

996. The method or combination of any one of embodiments 993 to 995, wherein the first variant Fc region comprises the pI variant substitutions N208D/Q295E/N384D/Q418E/N421D (pI_(−)_isosteric_A).

997. The method or combination of any one of embodiments 993 to 996, wherein the second variant Fc region comprises the pI variant substitutions N208D/Q295E/N384D/Q418E/N421D (pI_(−)_isosteric_A).

998. The method or combination of any one of embodiments 1 to 908, wherein the first MBM and/or second MBM comprises an Fc domain.

999. The method or combination of embodiment 998, wherein the Fc domain is an Fc heterodimer.

1000. The method or combination of embodiment 999, wherein the Fc heterodimer comprises any of the Fc modifications set forth in Table 3.

1001. The method or combination of embodiment 999, wherein the Fc heterodimer comprises knob-in-hole (“KIH”) modifications.

1002. The method or combination of embodiment 1001, wherein the KIH modifications are any of the KIH modifications described in Section 7.3 or in Table 3.

1003. The method or combination of embodiment 1001, wherein the KIH modifications are any of the alternative KIH modifications described in Section 7.3 or in Table 3.

1004. The method or combination of any one of embodiments 999 to 1003, wherein the first MBM and/or second MBM comprises polar bridge modifications.

1005. The method or combination of embodiment 1004, wherein the polar bridge modifications are any of the polar bridge modifications described in Section 7.3 or in Table 3.

1006. The method or combination of any one of embodiments to 999 to 1005, wherein the first MBM and/or second MBM comprises at least one of the Fc modifications designated as Fc 1 through Fc 150.

1007. The method or combination of embodiment 1006, wherein the first MBM and/or second MBM comprises at least one of the Fc modifications designated as Fc 1 through Fc 5.

1008. The method or combination of embodiment 1006, wherein the first MBM and/or second MBM comprises at least one of the Fc modifications designated as Fc 6 through Fc 10.

1009. The method or combination of embodiment 1006, wherein the first MBM and/or second MBM comprises at least one of the Fc modifications designated as Fc 11 through Fc 15.

1010. The method or combination of embodiment 1006, wherein the first MBM and/or second MBM comprises at least one of the Fc modifications designated as Fc 16 through Fc 20.

1011. The method or combination of embodiment 1006, wherein the first MBM and/or second MBM comprises at least one of the Fc modifications designated as Fc 21 through Fc 25.

1012. The method or combination of embodiment 1006, wherein the first MBM and/or second MBM comprises at least one of the Fc modifications designated as Fc 26 through Fc 30.

1013. The method or combination of embodiment 1006, wherein the first MBM and/or second MBM comprises at least one of the Fc modifications designated as Fc 31 through Fc 35.

1014. The method or combination of embodiment 1006, wherein the first MBM and/or second MBM comprises at least one of the Fc modifications designated as Fc 36 through Fc 40.

1015. The method or combination of embodiment 1006, wherein the first MBM and/or second MBM comprises at least one of the Fc modifications designated as Fc 41 through Fc 45.

1016. The method or combination of embodiment 1006, wherein the first MBM and/or second MBM comprises at least one of the Fc modifications designated as Fc 46 through Fc 50.

1017. The method or combination of embodiment 1006, wherein the first MBM and/or second MBM comprises at least one of the Fc modifications designated as Fc 51 through Fc 55.

1018. The method or combination of embodiment 1006, wherein the first MBM and/or second MBM comprises at least one of the Fc modifications designated as Fc 56 through Fc 60.

1019. The method or combination of embodiment 1006, wherein the first MBM and/or second MBM comprises at least one of the Fc modifications designated as Fc 61 through Fc 65.

1020. The method or combination of embodiment 1006, wherein the first MBM and/or second MBM comprises at least one of the Fc modifications designated as Fc 66 through Fc 70.

1021. The method or combination of embodiment 1006, wherein the first MBM and/or second MBM comprises at least one of the Fc modifications designated as Fc 71 through Fc 75.

1022. The method or combination of embodiment 1006, wherein the first MBM and/or second MBM comprises at least one of the Fc modifications designated as Fc 76 through Fc 80.

1023. The method or combination of embodiment 1006, wherein the first MBM and/or second MBM comprises at least one of the Fc modifications designated as Fc 81 through Fc 85.

1024. The method or combination of embodiment 1006, wherein the first MBM and/or second MBM comprises at least one of the Fc modifications designated as Fc 86 through Fc 90.

1025. The method or combination of embodiment 1006, wherein the first MBM and/or second MBM comprises at least one of the Fc modifications designated as Fc 91 through Fc 95.

1026. The method or combination of embodiment 1006, wherein the first MBM and/or second MBM comprises at least one of the Fc modifications designated as Fc 96 through Fc 100.

1027. The method or combination of embodiment 1006, wherein the first MBM and/or second MBM comprises at least one of the Fc modifications designated as Fc 101 through Fc 105.

1028. The method or combination of embodiment 1006, wherein the first MBM and/or second MBM comprises at least one of the Fc modifications designated as Fc 106 through Fc 110.

1029. The method or combination of embodiment 1006, wherein the first MBM and/or second MBM comprises at least one of the Fc modifications designated as Fc 111 through Fc 115.

1030. The method or combination of embodiment 1006, wherein the first MBM and/or second MBM comprises at least one of the Fc modifications designated as Fc 116 through Fc 120.

1031. The method or combination of embodiment 1006, wherein the first MBM and/or second MBM comprises at least one of the Fc modifications designated as Fc 121 through Fc 125.

1032. The method or combination of embodiment 1006, wherein the first MBM and/or second MBM comprises at least one of the Fc modifications designated as Fc 126 through Fc 130.

1033. The method or combination of embodiment 1006, wherein the first MBM and/or second MBM comprises at least one of the Fc modifications designated as Fc 131 through Fc 135.

1034. The method or combination of embodiment 1006, wherein the first MBM and/or second MBM comprises at least one of the Fc modifications designated as Fc 136 through Fc 140.

1035. The method or combination of embodiment 1006, wherein the first MBM and/or second MBM comprises at least one of the Fc modifications designated as Fc 141 through Fc 145.

1036. The method or combination of embodiment 1006, wherein the first MBM and/or second MBM comprises at least one of the Fc modifications designated as Fc 146 through Fc 150.

1037. The method or combination of any one of embodiments 998 to 1036, wherein the Fc domain has altered effector function.

1038. The method or combination of embodiment 1037, wherein the Fc domain has altered binding to one or more Fc receptors.

1039. The method or combination of embodiment 1038, wherein the one or more Fc receptors comprise FcRN.

1040. The method or combination of embodiment 1038 or embodiment 1039, wherein the one or more Fc receptors comprise leukocyte receptors.

1041. The method or combination of any one of embodiments 998 to 1040, wherein the Fc has modified disulfide bond architecture.

1042. The method or combination of any one of embodiments 998 to 1041, wherein the Fc has altered glycosylation patterns.

1043. The method or combination of any one of embodiments 998 to 1042, wherein the Fc comprises a hinge region.

1044. The method or combination of embodiment 1043, wherein the hinge region comprises any one of the hinge regions described in Section 7.3.2.

1045. The method or combination of embodiment 1044, wherein the hinge region comprises the amino acid sequence of the hinge region designated H1.

1046. The method or combination of embodiment 1044, wherein the hinge region comprises the amino acid sequence of the hinge region designated H2.

1047. The method or combination of embodiment 1044, wherein the hinge region comprises the amino acid sequence of the hinge region designated H3.

1048. The method or combination of embodiment 1044, wherein the hinge region comprises the amino acid sequence of the hinge region designated H4.

1049. The method or combination of embodiment 1044, wherein the hinge region comprises the amino acid sequence of the hinge region designated H5.

1050. The method or combination of embodiment 1044, wherein the hinge region comprises the amino acid sequence of the hinge region designated H6.

1051. The method or combination of embodiment 1044, wherein the hinge region comprises the amino acid sequence of the hinge region designated H7.

1052. The method or combination of embodiment 1044, wherein the hinge region comprises the amino acid sequence of the hinge region designated H8.

1053. The method or combination of embodiment 1044, wherein the hinge region comprises the amino acid sequence of the hinge region designated H9.

1054. The method or combination of embodiment 1044, wherein the hinge region comprises the amino acid sequence of the hinge region designated H10.

1055. The method or combination of embodiment 1044, wherein the hinge region comprises the amino acid sequence of the hinge region designated H11.

1056. The method or combination of embodiment 1044, wherein the hinge region comprises the amino acid sequence of the hinge region designated H12.

1057. The method or combination of embodiment 1044, wherein the hinge region comprises the amino acid sequence of the hinge region designated H13.

1058. The method or combination of embodiment 1044, wherein the hinge region comprises the amino acid sequence of the hinge region designated H14.

1059. The method or combination of embodiment 1044, wherein the hinge region comprises the amino acid sequence of the hinge region designated H15.

1060. The method or combination of embodiment 1044, wherein the hinge region comprises the amino acid sequence of the hinge region designated H16.

1061. The method or combination of embodiment 1044, wherein the hinge region comprises the amino acid sequence of the hinge region designated H17.

1062. The method or combination of embodiment 1044, wherein the hinge region comprises the amino acid sequence of the hinge region designated H18.

1063. The method or combination of embodiment 1044, wherein the hinge region comprises the amino acid sequence of the hinge region designated H19.

1064. The method or combination of embodiment 1044, wherein the hinge region comprises the amino acid sequence of the hinge region designated H20.

1065. The method or combination of embodiment 1044, wherein the hinge region comprises the amino acid sequence of the hinge region designated H21.

1066. The method or combination of any one of embodiments 1 to 1065, wherein the first MBM and/or second MBM comprises at least one scFv domain.

1067. The method or combination of embodiment 1066, wherein at least one scFv comprises a linker connecting the VH and VL domains.

1068. The method or combination of embodiment 1067, wherein the linker is 5 to 25 amino acids in length.

1069. The method or combination of embodiment 1068, wherein the linker is 12 to 20 amino acids in length.

1070. The method or combination of any one of embodiments 1067 to 1069, wherein the linker is a charged linker and/or a flexible linker.

1071. The method or combination of any one of embodiments 1067 to 1070, wherein the linker is selected from any one of linkers L1 through L54.

1072. The method or combination of embodiment 1071, wherein the linker region comprises the amino acid sequence of the linker designated L1.

1073. The method or combination of embodiment 1071, wherein the linker region comprises the amino acid sequence of the linker designated L2.

1074. The method or combination of embodiment 1071, wherein the linker region comprises the amino acid sequence of the linker designated L3.

1075. The method or combination of embodiment 1071, wherein the linker region comprises the amino acid sequence of the linker designated L4.

1076. The method or combination of embodiment 1071, wherein the linker region comprises the amino acid sequence of the linker designated L5.

1077. The method or combination of embodiment 1071, wherein the linker region comprises the amino acid sequence of the linker designated L6.

1078. The method or combination of embodiment 1071, wherein the linker region comprises the amino acid sequence of the linker designated L7.

1079. The method or combination of embodiment 1071, wherein the linker region comprises the amino acid sequence of the linker designated L8.

1080. The method or combination of embodiment 1071, wherein the linker region comprises the amino acid sequence of the linker designated L9.

1081. The method or combination of embodiment 1071, wherein the linker region comprises the amino acid sequence of the linker designated L10.

1082. The method or combination of embodiment 1071, wherein the linker region comprises the amino acid sequence of the linker designated L11.

1083. The method or combination of embodiment 1071, wherein the linker region comprises the amino acid sequence of the linker designated L12.

1084. The method or combination of embodiment 1071, wherein the linker region comprises the amino acid sequence of the linker designated L13.

1085. The method or combination of embodiment 1071, wherein the linker region comprises the amino acid sequence of the linker designated L14.

1086. The method or combination of embodiment 1071, wherein the linker region comprises the amino acid sequence of the linker designated L15.

1087. The method or combination of embodiment 1071, wherein the linker region comprises the amino acid sequence of the linker designated L16.

1088. The method or combination of embodiment 1071, wherein the linker region comprises the amino acid sequence of the linker designated L17.

1089. The method or combination of embodiment 1071, wherein the linker region comprises the amino acid sequence of the linker designated L18.

1090. The method or combination of embodiment 1071, wherein the linker region comprises the amino acid sequence of the linker designated L19.

1091. The method or combination of embodiment 1071, wherein the linker region comprises the amino acid sequence of the linker designated L20.

1092. The method or combination of embodiment 1071, wherein the linker region comprises the amino acid sequence of the linker designated L21.

1093. The method or combination of embodiment 1071, wherein the linker region comprises the amino acid sequence of the linker designated L22.

1094. The method or combination of embodiment 1071, wherein the linker region comprises the amino acid sequence of the linker designated L23.

1095. The method or combination of embodiment 1071, wherein the linker region comprises the amino acid sequence of the linker designated L24.

1096. The method or combination of embodiment 1071, wherein the linker region comprises the amino acid sequence of the linker designated L25.

1097. The method or combination of embodiment 1071, wherein the linker region comprises the amino acid sequence of the linker designated L26.

1098. The method or combination of embodiment 1071, wherein the linker region comprises the amino acid sequence of the linker designated L27.

1099. The method or combination of embodiment 1071, wherein the linker region comprises the amino acid sequence of the linker designated L28.

1100. The method or combination of embodiment 1071, wherein the linker region comprises the amino acid sequence of the linker designated L29.

1101. The method or combination of embodiment 1071, wherein the linker region comprises the amino acid sequence of the linker designated L30.

1102. The method or combination of embodiment 1071, wherein the linker region comprises the amino acid sequence of the linker designated L31.

1103. The method or combination of embodiment 1071, wherein the linker region comprises the amino acid sequence of the linker designated L32.

1104. The method or combination of embodiment 1071, wherein the linker region comprises the amino acid sequence of the linker designated L33.

1105. The method or combination of embodiment 1071, wherein the linker region comprises the amino acid sequence of the linker designated L34.

1106. The method or combination of embodiment 1071, wherein the linker region comprises the amino acid sequence of the linker designated L35.

1107. The method or combination of embodiment 1071, wherein the linker region comprises the amino acid sequence of the linker designated L36.

1108. The method or combination of embodiment 1071, wherein the linker region comprises the amino acid sequence of the linker designated L37.

1109. The method or combination of embodiment 1071, wherein the linker region comprises the amino acid sequence of the linker designated L38.

1110. The method or combination of embodiment 1071, wherein the linker region comprises the amino acid sequence of the linker designated L39.

1111. The method or combination of embodiment 1071, wherein the linker region comprises the amino acid sequence of the linker designated L40.

1112. The method or combination of embodiment 1071, wherein the linker region comprises the amino acid sequence of the linker designated L41.

1113. The method or combination of embodiment 1071, wherein the linker region comprises the amino acid sequence of the linker designated L42.

1114. The method or combination of embodiment 1071, wherein the linker region comprises the amino acid sequence of the linker designated L43.

1115. The method or combination of embodiment 1071, wherein the linker region comprises the amino acid sequence of the linker designated L44.

1116. The method or combination of embodiment 1071, wherein the linker region comprises the amino acid sequence of the linker designated L45.

1117. The method or combination of embodiment 1071, wherein the linker region comprises the amino acid sequence of the linker designated L46.

1118. The method or combination of embodiment 1071, wherein the linker region comprises the amino acid sequence of the linker designated L47.

1119. The method or combination of embodiment 1071, wherein the linker region comprises the amino acid sequence of the linker designated L48.

1120. The method or combination of embodiment 1071, wherein the linker region comprises the amino acid sequence of the linker designated L49.

1121. The method or combination of embodiment 1071, wherein the linker region comprises the amino acid sequence of the linker designated L50.

1122. The method or combination of embodiment 1071, wherein the linker region comprises the amino acid sequence of the linker designated L51.

1123. The method or combination of embodiment 1071, wherein the linker region comprises the amino acid sequence of the linker designated L52.

1124. The method or combination of embodiment 1071, wherein the linker region comprises the amino acid sequence of the linker designated L53.

1125. The method or combination of embodiment 1071, wherein the linker region comprises the amino acid sequence of the linker designated L54.

1126. The method or combination of any one of embodiments 1 to 1125, wherein the first MBM and/or second MBM comprises at least one Fab domain.

1127. The method or combination of embodiment 1126, wherein at least one Fab domain comprises any of the Fab heterodimerization modifications set forth in Table 1.

1128. The method or combination of embodiment 1127, wherein at least one Fab domain comprises the Fab heterodimerization modifications designated as F1.

1129. The method or combination of embodiment 1127, wherein at least one Fab domain comprises the Fab heterodimerization modifications designated as F2.

1130. The method or combination of embodiment 1127, wherein at least one Fab domain comprises the Fab heterodimerization modifications designated as F3.

1131. The method or combination of embodiment 1127, wherein at least one Fab domain comprises the Fab heterodimerization modifications designated as F4.

1132. The method or combination of embodiment 1127, wherein at least one Fab domain comprises the Fab heterodimerization modifications designated as F5.

1133. The method or combination of embodiment 1127, wherein at least one Fab domain comprises the Fab heterodimerization modifications designated as F6.

1134. The method or combination of embodiment 1127, wherein at least one Fab domain comprises the Fab heterodimerization modifications designated as F7.

1135. The method or combination of any one of embodiments 1 to 1134, wherein the first MBM and/or second MBM comprises at least two ABMs, an ABM and an ABM chain, or two ABM chains connected to one another via a linker.

1136. The method or combination of embodiment 1135, wherein the linker is 5 to 25 amino acids in length.

1137. The method or combination of embodiment 1136, wherein the linker is 12 to 20 amino acids in length.

1138. The method or combination of any one of embodiments 1135 to 1137, wherein the linker is a charged linker and/or a flexible linker.

1139. The method or combination of any one of embodiments 1135 to 1138, wherein the linker is selected from any one of linkers L1 through L54.

1140. The method or combination of embodiment 1139, wherein the linker region comprises the amino acid sequence of the linker designated L1.

1141. The method or combination of embodiment 1139, wherein the linker region comprises the amino acid sequence of the linker designated L2.

1142. The method or combination of embodiment 1139, wherein the linker region comprises the amino acid sequence of the linker designated L3.

1143. The method or combination of embodiment 1139, wherein the linker region comprises the amino acid sequence of the linker designated L4.

1144. The method or combination of embodiment 1139, wherein the linker region comprises the amino acid sequence of the linker designated L5.

1145. The method or combination of embodiment 1139, wherein the linker region comprises the amino acid sequence of the linker designated L6.

1146. The method or combination of embodiment 1139, wherein the linker region comprises the amino acid sequence of the linker designated L7.

1147. The method or combination of embodiment 1139, wherein the linker region comprises the amino acid sequence of the linker designated L8.

1148. The method or combination of embodiment 1139, wherein the linker region comprises the amino acid sequence of the linker designated L9.

1149. The method or combination of embodiment 1139, wherein the linker region comprises the amino acid sequence of the linker designated L10.

1150. The method or combination of embodiment 1139, wherein the linker region comprises the amino acid sequence of the linker designated L11.

1151. The method or combination of embodiment 1139, wherein the linker region comprises the amino acid sequence of the linker designated L12.

1152. The method or combination of embodiment 1139, wherein the linker region comprises the amino acid sequence of the linker designated L13.

1153. The method or combination of embodiment 1139, wherein the linker region comprises the amino acid sequence of the linker designated L14.

1154. The method or combination of embodiment 1139, wherein the linker region comprises the amino acid sequence of the linker designated L15.

1155. The method or combination of embodiment 1139, wherein the linker region comprises the amino acid sequence of the linker designated L16.

1156. The method or combination of embodiment 1139, wherein the linker region comprises the amino acid sequence of the linker designated L17.

1157. The method or combination of embodiment 1139, wherein the linker region comprises the amino acid sequence of the linker designated L18.

1158. The method or combination of embodiment 1139, wherein the linker region comprises the amino acid sequence of the linker designated L19.

1159. The method or combination of embodiment 1139, wherein the linker region comprises the amino acid sequence of the linker designated L20.

1160. The method or combination of embodiment 1139, wherein the linker region comprises the amino acid sequence of the linker designated L21.

1161. The method or combination of embodiment 1139, wherein the linker region comprises the amino acid sequence of the linker designated L22.

1162. The method or combination of embodiment 1139, wherein the linker region comprises the amino acid sequence of the linker designated L23.

1163. The method or combination of embodiment 1139, wherein the linker region comprises the amino acid sequence of the linker designated L24.

1164. The method or combination of embodiment 1139, wherein the linker region comprises the amino acid sequence of the linker designated L25.

1165. The method or combination of embodiment 1139, wherein the linker region comprises the amino acid sequence of the linker designated L26.

1166. The method or combination of embodiment 1139, wherein the linker region comprises the amino acid sequence of the linker designated L27.

1167. The method or combination of embodiment 1139, wherein the linker region comprises the amino acid sequence of the linker designated L28.

1168. The method or combination of embodiment 1139, wherein the linker region comprises the amino acid sequence of the linker designated L29.

1169. The method or combination of embodiment 1139, wherein the linker region comprises the amino acid sequence of the linker designated L30.

1170. The method or combination of embodiment 1139, wherein the linker region comprises the amino acid sequence of the linker designated L31.

1171. The method or combination of embodiment 1139, wherein the linker region comprises the amino acid sequence of the linker designated L32.

1172. The method or combination of embodiment 1139, wherein the linker region comprises the amino acid sequence of the linker designated L33.

1173. The method or combination of embodiment 1139, wherein the linker region comprises the amino acid sequence of the linker designated L34.

1174. The method or combination of embodiment 1139, wherein the linker region comprises the amino acid sequence of the linker designated L35.

1175. The method or combination of embodiment 1139, wherein the linker region comprises the amino acid sequence of the linker designated L36.

1176. The method or combination of embodiment 1139, wherein the linker region comprises the amino acid sequence of the linker designated L37.

1177. The method or combination of embodiment 1139, wherein the linker region comprises the amino acid sequence of the linker designated L38.

1178. The method or combination of embodiment 1139, wherein the linker region comprises the amino acid sequence of the linker designated L39.

1179. The method or combination of embodiment 1139, wherein the linker region comprises the amino acid sequence of the linker designated L40.

1180. The method or combination of embodiment 1139, wherein the linker region comprises the amino acid sequence of the linker designated L41.

1181. The method or combination of embodiment 1139, wherein the linker region comprises the amino acid sequence of the linker designated L42.

1182. The method or combination of embodiment 1139, wherein the linker region comprises the amino acid sequence of the linker designated L43.

1183. The method or combination of embodiment 1139, wherein the linker region comprises the amino acid sequence of the linker designated L44.

1184. The method or combination of embodiment 1139, wherein the linker region comprises the amino acid sequence of the linker designated L45.

1185. The method or combination of embodiment 1139, wherein the linker region comprises the amino acid sequence of the linker designated L46.

1186. The method or combination of embodiment 1139, wherein the linker region comprises the amino acid sequence of the linker designated L47.

1187. The method or combination of embodiment 1139, wherein the linker region comprises the amino acid sequence of the linker designated L48.

1188. The method or combination of embodiment 1139, wherein the linker region comprises the amino acid sequence of the linker designated L49.

1189. The method or combination of embodiment 1071, wherein the linker region comprises the amino acid sequence of the linker designated L50.

1190. The method or combination of embodiment 1071, wherein the linker region comprises the amino acid sequence of the linker designated L51.

1191. The method or combination of embodiment 1071, wherein the linker region comprises the amino acid sequence of the linker designated L52.

1192. The method or combination of embodiment 1071, wherein the linker region comprises the amino acid sequence of the linker designated L53.

1193. The method or combination of embodiment 1139, wherein the linker region comprises the amino acid sequence of the linker designated L54.

1194. The method or combination of any one of embodiments 1 to 1193, wherein when the first MBM does not comprise an ABM2 or does not comprise an ABM3, the first MBM is a bispecific binding molecule (“BBM”).

1195. The method or combination of any one of embodiments 1 to 1194, wherein when the second MBM does not comprise an ABM5 or does not comprise an ABM6, the second MBM is a BBM.

1196. The method or combination of embodiment 1194 or embodiment 1195, wherein the first MBM and/or second MBM is bivalent.

1197. The method or combination of embodiment 1196, wherein the first MBM and/or second MBM, each independent of the other, has any one of the configurations depicted in FIGS. 1B-1F.

1198. The method or combination of embodiment 1197, wherein the first MBM and/or second MBM has the configuration depicted in FIG. 1B.

1199. The method or combination of embodiment 1197, wherein the first MBM and/or second MBM has the configuration depicted in FIG. 1C.

1200. The method or combination of embodiment 1197, wherein the first MBM and/or second MBM has the configuration depicted in FIG. 1D.

1201. The method or combination of embodiment 1197, wherein the first MBM and/or second MBM has the configuration depicted in FIG. 1E.

1202. The method or combination of embodiment 1197, wherein the first MBM and/or second MBM has the configuration depicted in FIG. 1F.

1203. The method or combination of any one of embodiments 1197 to 1202, in which the ABMs of the first MBM and/or second MBM have the configuration designated as B1.

1204. The method or combination of any one of embodiments 1197 to 1202, in which the ABMs of the first MBM and/or second MBM have the configuration designated as B2.

1205. The method or combination of embodiment 1194 or embodiment 1195, wherein the first MBM and/or the second MBM is trivalent.

1206. The method or combination of embodiment 1205, wherein the first MBM and/or second MBM, each independent of the other, has any one of the configurations depicted in FIGS. 1G-1Z.

1207. The method or combination of embodiment 1206, wherein the first MBM and/or second MBM has the configuration depicted in FIG. 1G.

1208. The method or combination of embodiment 1206, wherein the first MBM and/or second MBM has the configuration depicted in FIG. 1H.

1209. The method or combination of embodiment 1206, wherein the first MBM and/or second MBM has the configuration depicted in FIG. 1I.

1210. The method or combination of embodiment 1206, wherein the first MBM and/or second MBM has the configuration depicted in FIG. 1J.

1211. The method or combination of embodiment 1206, wherein the first MBM and/or second MBM has the configuration depicted in FIG. 1K.

1212. The method or combination of embodiment 1206, wherein the first MBM and/or second MBM has the configuration depicted in FIG. 1L.

1213. The method or combination of embodiment 1206, wherein the first MBM and/or second MBM has the configuration depicted in FIG. 1M.

1214. The method or combination of embodiment 1206, wherein the first MBM and/or second MBM has the configuration depicted in FIG. 1N.

1215. The method or combination of embodiment 1206, wherein the first MBM and/or second MBM has the configuration depicted in FIG. 1O.

1216. The method or combination of embodiment 1206, wherein the first MBM and/or second MBM has the configuration depicted in FIG. 1P.

1217. The method or combination of embodiment 1206, wherein the first MBM and/or second MBM has the configuration depicted in FIG. 1Q.

1218. The method or combination of embodiment 1206, wherein the first MBM and/or second MBM has the configuration depicted in FIG. 1R.

1219. The method or combination of embodiment 1206, wherein the first MBM and/or second MBM has the configuration depicted in FIG. 1s.

1220. The method or combination of embodiment 1206, wherein the first MBM and/or second MBM has the configuration depicted in FIG. 1T.

1221. The method or combination of embodiment 1206, wherein the first MBM and/or second MBM has the configuration depicted in FIG. 1U.

1222. The method or combination of embodiment 1206, wherein the first MBM and/or second MBM has the configuration depicted in FIG. 1V.

1223. The method or combination of embodiment 1206, wherein the first MBM and/or second MBM has the configuration depicted in FIG. 1W.

1224. The method or combination of embodiment 1206, wherein the first MBM and/or second MBM has the configuration depicted in FIG. 1X.

1225. The method or combination of embodiment 1206, wherein the first MBM and/or second MBM has the configuration depicted in FIG. 1Y.

1226. The method or combination of embodiment 1206, wherein the first MBM and/or second MBM has the configuration depicted in FIG. 1Z.

1227. The method or combination of any one of embodiments 1206 to 1226, in which the ABMs of the first MBM and/or second MBM have the configuration designated as T1.

1228. The method or combination of any one of embodiments 1206 to 1226, in which the ABMs of the first MBM and/or second MBM have the configuration designated as T2.

1229. The method or combination of any one of embodiments 1206 to 1226, in which the ABMs of the first MBM and/or second MBM have the configuration designated as T3.

1230. The method or combination of any one of embodiments 1206 to 1226, in which the ABMs of the first MBM and/or second MBM have the configuration designated as T4.

1231. The method or combination of any one of embodiments 1206 to 1226, in which the ABMs of the first MBM and/or second MBM have the configuration designated as T5.

1232. The method or combination of any one of embodiments 1206 to 1226, in which the ABMs of the first MBM and/or second MBM have the configuration designated as T6.

1233. The method or combination of embodiment 1194 or embodiment 1195, wherein the first MBM and/or second MBM is tetravalent.

1234. The method or combination of embodiment 1233, wherein the first MBM and/or second MBM, each independent of the other, has any one of the configurations depicted in FIGS. 1AA-1AH.

1235. The method or combination of embodiment 1234, wherein the first MBM and/or second MBM has the configuration depicted in FIG. 1AA.

1236. The method or combination of embodiment 1234, wherein the first MBM and/or second MBM has the configuration depicted in FIG. 1AB.

1237. The method or combination of embodiment 1234, wherein the first MBM and/or second MBM has the configuration depicted in FIG. 1AC.

1238. The method or combination of embodiment 1234, wherein the first MBM and/or second MBM has the configuration depicted in FIG. 1AD.

1239. The method or combination of embodiment 1234, wherein the first MBM and/or second MBM has the configuration depicted in FIG. 1AE.

1240. The method or combination of embodiment 1234, wherein the first MBM and/or second MBM has the configuration depicted in FIG. 1AF.

1241. The method or combination of embodiment 1234, wherein the first MBM and/or second MBM has the configuration depicted in FIG. 1AG.

1242. The method or combination of embodiment 1234, wherein the first MBM and/or second MBM has the configuration depicted in FIG. 1AH.

1243. The method or combination of any one of embodiments 1234 to 1242, in which the ABMs of the first MBM and/or second MBM have any one of the configurations designated Tv 1 through Tv 24.

1244. The method or combination of embodiment 1243, in which the ABMs of the first MBM and/or second MBM have the configuration designated Tv 1.

1245. The method or combination of embodiment 1243, in which the ABMs of the first MBM and/or second MBM have the configuration designated Tv 2.

1246. The method or combination of embodiment 1243, in which the ABMs of the first MBM and/or second MBM have the configuration designated Tv 3.

1247. The method or combination of embodiment 1243, in which the ABMs of the first MBM and/or second MBM have the configuration designated Tv 4.

1248. The method or combination of embodiment 1243, in which the ABMs of the first MBM and/or second MBM have the configuration designated Tv 5.

1249. The method or combination of embodiment 1243, in which the ABMs of the first MBM and/or second MBM have the configuration designated Tv 6.

1250. The method or combination of embodiment 1243, in which the ABMs of the first MBM and/or second MBM have the configuration designated Tv 7.

1251. The method or combination of embodiment 1243, in which the ABMs of the first MBM and/or second MBM have the configuration designated Tv 8.

1252. The method or combination of embodiment 1243, in which the ABMs of the first MBM and/or second MBM have the configuration designated Tv 9.

1253. The method or combination of embodiment 1243, in which the ABMs of the first MBM and/or second MBM have the configuration designated Tv 10.

1254. The method or combination of embodiment 1243, in which the ABMs of the first MBM and/or second MBM have the configuration designated Tv 11.

1255. The method or combination of embodiment 1243, in which the ABMs of the first MBM and/or second MBM have the configuration designated Tv 12.

1256. The method or combination of embodiment 1243, in which the ABMs of the first MBM and/or second MBM have the configuration designated Tv 13.

1257. The method or combination of embodiment 1243, in which the ABMs of the first MBM and/or second MBM have the configuration designated Tv 14.

1258. The method or combination of any one of embodiments 1 to 1193, wherein when the first MBM comprises an ABM1, an ABM2, and an ABM3, the first MBM is a trispecific binding molecule (“TBM”).

1259. The method or combination of any one of embodiments 1 to 1193 and 1258, wherein when the second MBM comprises an ABM4, an ABM5, and an ABM6, the second MBM is a TBM.

1260. The method or combination of embodiment 1258 or embodiment 1259, wherein the first MBM and/or second MBM is trivalent.

1261. The method or combination of embodiment 1260, wherein the first MBM and/or second MBM, each independent of the other, has any one of the configurations depicted in FIGS. 2B-2P.

1262. The method or combination of embodiment 1261, wherein the first MBM and/or second MBM has the configuration depicted in FIG. 2B.

1263. The method or combination of embodiment 1261, wherein the first MBM and/or second MBM has the configuration depicted in FIG. 2C.

1264. The method or combination of embodiment 1261, wherein the first MBM and/or second MBM has the configuration depicted in FIG. 2D.

1265. The method or combination of embodiment 1261, wherein the first MBM and/or second MBM has the configuration depicted in FIG. 2E.

1266. The method or combination of embodiment 1261, wherein the first MBM and/or second MBM has the configuration depicted in FIG. 2F.

1267. The method or combination of embodiment 1261, wherein the first MBM and/or second MBM has the configuration depicted in FIG. 2G.

1268. The method or combination of embodiment 1261, wherein the first MBM and/or second MBM has the configuration depicted in FIG. 2H.

1269. The method or combination of embodiment 1261, wherein the first MBM and/or second MBM has the configuration depicted in FIG. 2I.

1270. The method or combination of embodiment 1261, wherein the first MBM and/or second MBM has the configuration depicted in FIG. 2J.

1271. The method or combination of embodiment 1261, wherein the first MBM and/or second MBM has the configuration depicted in FIG. 2K.

1272. The method or combination of embodiment 1261, wherein the first MBM and/or second MBM has the configuration depicted in FIG. 2L.

1273. The method or combination of embodiment 1261, wherein the first MBM and/or second MBM has the configuration depicted in FIG. 2M.

1274. The method or combination of embodiment 1261, wherein the first MBM and/or second MBM has the configuration depicted in FIG. 2N.

1275. The method or combination of embodiment 1261, wherein the first MBM and/or second MBM has the configuration depicted in FIG. 2O.

1276. The method or combination of embodiment 1261, wherein the first MBM and/or second MBM has the configuration depicted in FIG. 2P.

1277. The method or combination of any one of embodiments 1260 to 1276, wherein the first MBM and/or second MBM has the configuration referred to as T1.

1278. The method or combination of any one of embodiments 1260 to 1276, wherein the first MBM and/or second MBM has the configuration referred to as T2.

1279. The method or combination of any one of embodiments 1260 to 1276, wherein the first MBM and/or second MBM has the configuration referred to as T3.

1280. The method or combination of any one of embodiments 1260 to 1276, wherein the first MBM and/or second MBM has the configuration referred to as T4.

1281. The method or combination of any one of embodiments 1260 to 1276, wherein the first MBM and/or second MBM has the configuration referred to as T5.

1282. The method or combination of any one of embodiments 1260 to 1276, wherein the first MBM and/or second MBM has the configuration referred to as T6.

1283. The method or combination of embodiment 1258 or embodiment 1259, wherein the first MBM and/or second MBM is tetravalent.

1284. The method or combination of embodiment 1283, wherein the first MBM and/or second MBM, each independent of the other, has any one of the configurations depicted in FIGS. 2Q-2S.

1285. The method or combination of embodiment 1284, wherein the first MBM and/or second MBM has the configuration depicted in FIG. 2Q.

1286. The method or combination of embodiment 1284, wherein the first MBM and/or second MBM has the configuration depicted in FIG. 2R.

1287. The method or combination of embodiment 1284, wherein the first MBM and/or second MBM has the configuration depicted in FIG. 2S.

1288. The method or combination of any one of embodiments 1283 to 1287, wherein the first MBM and/or the second MBM, each independent of the other, has any one of the configurations referred to as Tv1 through Tv24.

1289. The method or combination of embodiment 1258 or embodiment 1259, wherein the first MBM and/or second MBM is pentavalent.

1290. The method or combination of embodiment 1289, wherein the first MBM and/or the second MBM has the configuration depicted in FIG. 2T.

1291. The method or combination of embodiment 1289 or embodiment 1290, wherein the first MBM and/or the second MBM, each independent of the other, has any one of the configurations referred to as Pv1 through Pv100.

1292. The method or combination of embodiment 1258 or embodiment 1259, wherein the first MBM and/or the second MBM is hexavalent.

1293. The method or combination of embodiment 1292, wherein the first MBM and/or the second MBM, each independent of the other, has any one of the configurations depicted in FIGS. 2U-2V.

1294. The method or combination of embodiment 1293, wherein the first MBM and/or the second MBM has the configuration depicted in FIG. 2U.

1295. The method or combination of embodiment 1293, wherein the first MBM and/or the second MBM has the configuration depicted in FIG. 2V.

1296. The method or combination of any one of embodiments 1292 to 1295, wherein the first MBM and/or the second MBM, each independent of the other, has any of the configurations referred to as Hv1 through Hv330.

1297. The method or combination of any one of embodiments 1 to 1296, wherein each antigen-binding module of the first MBM is capable of binding its respective target at the same time as each of the other antigen-binding modules of the first MBM is bound to its respective target.

1298. The method or combination of any one of embodiments 1 to 1297, wherein each antigen-binding module of the second MBM is capable of binding its respective target at the same time as each of the other antigen-binding modules of the second MBM is bound to its respective target.

1299. The method or combination of any one of embodiments 1 to 1298, wherein any one, any two, three, four, five, or six of ABM1, ABM2, when present, ABM3, when present, ABM4, ABM5, when present, and ABM6, when present, has cross-species reactivity.

1300. The method or combination of embodiment 1299, wherein ABM1 further binds specifically to CD2 in one or more non-human mammalian species.

1301. The method or combination of embodiment 1299 or embodiment 1300, wherein ABM2, when present, further binds specifically to the TAA in one or more non-human mammalian species.

1302. The method or combination of any one of embodiments 1297 to 1301, wherein ABM3, when present, further binds specifically to the TMEA in one or more non-human mammalian species.

1303. The method or combination of any one of embodiments 1297 to 1302, wherein ABM4 further binds specifically to the component of a TCR complex or secondary T-cell signaling molecule in one or more non-human mammalian species.

1304. The method or combination of any one of embodiments 1297 to 1303, wherein ABM5, when present, further binds specifically to the TAA in one or more non-human mammalian species.

1305. The method or combination of any one of embodiments 1297 to 1304, wherein ABM6, when present, further binds specifically to the TMEA in one or more non-human mammalian species.

1306. The method or combination of any one of embodiments 1299 to 1305, wherein the one or more non-human mammalian species comprises one or more non-human primate species.

1307. The method or combination of embodiment 1306, wherein the one or more non-human primate species comprises Macaca fascicularis.

1308. The method or combination of embodiment 1306, wherein the one or more non-human primate species comprises Macaca mulatta.

1309. The method or combination of embodiment 1306, wherein the one or more non-human primate species comprises Macaca nemestrina.

1310. The method or combination of any one of embodiments 1299 to 1309, wherein the one or more non-human mammalian species comprises Mus musculus.

1311. The method or combination of any one of embodiments 1 to 1298, wherein any one, any two, three, four, five, or all six of ABM1, ABM2 ABM3, ABM4, ABM5, and ABM6 does not have cross-species reactivity.

1312. The method of embodiment 1 or the combination of embodiment 2, wherein the first MBM comprises polypeptides having the amino acid sequences of CD22_HA22-CD58 Bispecific set forth in Table 28.

1313. The method of embodiment 1 or the combination of embodiment 2, wherein the first MBM comprises polypeptides having the amino acid sequences of CD22_65-CD58 Bispecific set forth in Table 28.

1314. The method of embodiment 1 or the combination of embodiment 2, wherein the first MBM comprises polypeptides having the amino acid sequences of CD22_M971-CD58 Bispecific set forth in Table 28.

1315. The method of embodiment 1 or the combination of embodiment 2, wherein the first MBM comprises polypeptides having the amino acid sequences of CD22_HA22 CD2 Bispecific set forth in Table 28.

1316. The method of embodiment 1 or the combination of embodiment 2, wherein the first MBM comprises polypeptides having the amino acid sequences of CD22_65-CD2 Bispecific set forth in Table 28.

1317. The method of embodiment 1 or the combination of embodiment 2, wherein the first MBM comprises polypeptides having the amino acid sequences of CD22_M971-CD2 Bispecific set forth in Table 28.

1318. The method of embodiment 1 or the combination of embodiment 2, wherein the first MBM comprises polypeptides having the amino acid sequences of CD20_1-CD58 Bispecific set forth in Table 28.

1319. The method of embodiment 1 or the combination of embodiment 2, wherein the first MBM comprises polypeptides having the amino acid sequences of CD20_1-CD2_2 Bispecific set forth in Table 28.

1320. The method of embodiment 1 or the combination of embodiment 2, wherein the first MBM comprises polypeptides having the amino acid sequences of MSLN_SS1-CD58-Bispecific set forth in Table 29.

1321. The method of embodiment 1 or the combination of embodiment 2, wherein the first MBM comprises polypeptides having the amino acid sequences of MSLN_M5-CD58 Bispecific set forth in Table 29.

1322. The method of embodiment 1 or the combination of embodiment 2, wherein the first MBM comprises polypeptides having the amino acid sequences of MSLN_SS1-CD2 Bispecific set forth in Table 29.

1323. The method of embodiment 1 or the combination of embodiment 2, wherein the first MBM comprises polypeptides having the amino acid sequences of MSLN_M5-CD2 Bispecific set forth in Table 29.

1324. The method of embodiment 1 or the combination of embodiment 2, wherein the first MBM comprises polypeptides having the amino acid sequences of CD2×CD20 BBM set forth in Table 30A.

1325. The method of embodiment 1 or the combination of embodiment 2 or the method or combination of any one of embodiments 1312 to 1324, wherein the second MBM comprises polypeptides having the amino acid sequences of CD22_HA22-CD3 Bispecific set forth in Table 28.

1326. The method of embodiment 1 or the combination of embodiment 2 or the method or combination of any one of embodiments 1312 to 1324, wherein the second MBM comprises polypeptides having the amino acid sequences of CD22_M971-CD3 Bispecific set forth in Table 28.

1327. The method of embodiment 1 or the combination of embodiment 2 or the method or combination of any one of embodiments 1312 to 1324, wherein the second MBM comprises polypeptides having the amino acid sequences of CD22_M971-CD3 Bispecific set forth in Table 28.

1328. The method of embodiment 1 or the combination of embodiment 2 or the method or combination of any one of embodiments 1312 to 1324, wherein the second MBM comprises polypeptides having the amino acid sequences of MSLN_SS1-CD3-16 nM Bispecific set forth in Table 29.

1329. The method of embodiment 1 or the combination of embodiment 2 or the method or combination of any one of embodiments 1312 to 1324, wherein the second MBM comprises polypeptides having the amino acid sequences of MSLN_M5-CD3-16 nM Bispecific set forth in Table 29.

1330. The method of embodiment 1 or the combination of embodiment 2 or the method or combination of any one of embodiments 1312 to 1324, wherein the second MBM comprises polypeptides having the amino acid sequences of CD3×CD19 BBM set forth in Table 30B.

1331. The method or combination of any one of embodiments 1 to 1330, wherein the first MBM and/or second MBM is conjugated to an agent, optionally a therapeutic agent, a diagnostic agent, a masking moiety, a cleavable moiety, or any combination thereof.

I332. The method or combination of embodiment 1331, wherein the agent is a cytotoxic or cytostatic agent.

1333. The method or combination of embodiment 1332, wherein the agent is any one of the agents described in Section 7.12.

1334. The method or combination of any one of embodiments 1331 to 1333, wherein the first and/or second MBM is conjugated to a radionuclide.

1335. The method or combination of any one of embodiments 1331 to 1333, wherein the first and/or second MBM is conjugated to an alkylating agent.

1336. The method or combination of any one of embodiments 1331 to 1333, wherein the first and/or second MBM is conjugated to a topoisomerase inhibitor, which is optionally a topoisomerase I inhibitor or a topoisomerase II inhibitor.

1337. The method or combination of any one of embodiments 1331 to 1333, wherein the first and/or second MBM is conjugated to a DNA damaging agent.

1338. The method or combination of any one of embodiments 1331 to 1333, wherein the first and/or second MBM is conjugated to a DNA intercalating agent, optionally a groove binding agent such as a minor groove binding agent.

1339. The method or combination of any one of embodiments 1331 to 1333, wherein the first and/or second MBM is conjugated to a RNA/DNA antimetabolite.

1340. The method or combination of any one of embodiments 1331 to 1333, wherein the first and/or second MBM is conjugated to a kinase inhibitor.

1341. The method or combination of any one of embodiments 1331 to 1333, wherein the first and/or second MBM is conjugated to a protein synthesis inhibitor.

1342. The method or combination of any one of embodiments 1331 to 1333, wherein the first and/or second MBM is conjugated to a histone deacetylase (HDAC) inhibitor.

1343. The method or combination of any one of embodiments 1331 to 1333, wherein the first and/or second MBM is conjugated to a mitochondrial inhibitor, which is optionally an inhibitor of a phosphoryl transfer reaction in mitochondria.

1344. The method or combination of any one of embodiments 1331 to 1333, wherein the first and/or second MBM is conjugated to an antimitotic agent.

1345. The method or combination of any one of embodiments 1331 to 1333, wherein the first and/or second MBM is conjugated to a maytansinoid.

1346. The method or combination of any one of embodiments 1331 to 1333, wherein the first and/or second MBM is conjugated to a kinesin inhibitor.

1347. The method or combination of any one of embodiments 1331 to 1333, wherein the first and/or second MBM is conjugated to a kinesin-like protein KI F11 inhibitor.

1348. The method or combination of any one of embodiments 1331 to 1333, wherein the first and/or second MBM is conjugated to a V-ATPase (vacuolar-type H+-ATPase) inhibitor.

1349. The method or combination of any one of embodiments 1331 to 1333, wherein the first and/or second MBM is conjugated to a pro-apoptotic agent.

1350. The method or combination of any one of embodiments 1331 to 1333, wherein the first and/or second MBM is conjugated to a Bcl2 (B-cell lymphoma 2) inhibitor.

1351. The method or combination of any one of embodiments 1331 to 1333, wherein the first and/or second MBM is conjugated to an MCL1 (myeloid cell leukemia 1) inhibitor.

1352. The method or combination of any one of embodiments 1331 to 1333, wherein the first and/or second MBM is conjugated to a HSP90 (heat shock protein 90) inhibitor.

1353. The method or combination of any one of embodiments 1331 to 1333, wherein the first and/or second MBM is conjugated to an IAP (inhibitor of apoptosis) inhibitor.

1354. The method or combination of any one of embodiments 1331 to 1333, wherein the first and/or second MBM is conjugated to an mTOR (mechanistic target of rapamycin) inhibitor.

1355. The method or combination of any one of embodiments 1331 to 1333, wherein the first and/or second MBM is conjugated to a microtubule stabilizer.

1356. The method or combination of any one of embodiments 1331 to 1333, wherein the first and/or second MBM is conjugated to a microtubule destabilizer.

1357. The method or combination of any one of embodiments 1331 to 1333, wherein the first and/or second MBM is conjugated to an auristatin.

1358. The method or combination of any one of embodiments 1331 to 1333, wherein the first and/or second MBM is conjugated to a dolastatin.

1359. The method or combination of any one of embodiments 1331 to 1333, wherein the first and/or second MBM is conjugated to a MetAP (methionine aminopeptidase).

1360. The method or combination of any one of embodiments 1331 to 1333, wherein the first and/or second MBM is conjugated to a CRM1 (chromosomal maintenance 1) inhibitor.

1361. The method or combination of any one of embodiments 1331 to 1333, wherein the first and/or second MBM is conjugated to a DPPIV (dipeptidyl peptidase IV) inhibitor.

1362. The method or combination of any one of embodiments 1331 to 1333, wherein the first and/or second MBM is conjugated to a proteasome inhibitor.

1363. The method or combination of any one of embodiments 1331 to 1333, wherein the first and/or second MBM is conjugated to a protein synthesis inhibitor.

1364. The method or combination of any one of embodiments 1331 to 1333, wherein the first and/or second MBM is conjugated to a CDK2 (cyclin-dependent kinase 2) inhibitor.

1365. The method or combination of any one of embodiments 1331 to 1333, wherein the first and/or second MBM is conjugated to a CDK9 (cyclin-dependent kinase 9) inhibitor.

1366. The method or combination of any one of embodiments 1331 to 1333, wherein the first and/or second MBM is conjugated to a RNA polymerase inhibitor.

1367. The method or combination of any one of embodiments 1331 to 1333, wherein the first and/or second MBM is conjugated to a DHFR (dihydrofolate reductase) inhibitor.

1368. The method or combination of any one of embodiments 1331 to 1367, wherein the agent is attached to the first and/or second MBM with a linker, which is optionally a cleavable linker or a non-cleavable linker, e.g., a linker as described in Section 7.12.2.

1369. The method or combination of any one of embodiments 1331 to 1368, wherein the cytotoxic or cytostatic agent is conjugated to the first and/or second MBM via a linker as described in Section 7.12.2.

1370. The method or combination of any one of embodiments 1 to 1369, wherein the subject has a proliferative disease.

1371. The method or combination of embodiment 1370, wherein the proliferative disease is a cancer or a precancerous condition.

1372. The method or combination of embodiment 1370 or 1371, wherein the proliferative disease is a hematologic proliferative disease.

1373. The method or combination of 1372, wherein the proliferative disease is a lymphoma, a leukemia, multiple myeloma, a chronic myeloproliferative neoplasm, a macroglobulinemia, a myelodysplastic syndrome, a myelodysplastic/myeloproliferative neoplasm, or a plasmacytic dendritic cell neoplasm.

1374. The method or combination of embodiment 1373, wherein the proliferative disease is a lymphoma.

1375. The method or combination of embodiment 1374, wherein the lymphoma is Hodgkin's lymphoma.

1376. The method or combination of embodiment 1375, wherein the Hodgkin's lymphoma is nodular sclerosing Hodgkin's lymphoma, mixed-cellularity subtype Hodgkin's lymphoma, lymphocyte-rich or lymphocytic predominance Hodgkin's lymphoma, or lymphocyte depleted Hodgkin's lymphoma.

1377. The method or combination of embodiment 1376, wherein the Hodgkin's lymphoma is nodular sclerosing Hodgkin's lymphoma.

1378. The method or combination of embodiment 1376, wherein the Hodgkin's lymphoma is mixed-cellularity subtype Hodgkin's lymphoma.

1379. The method or combination of embodiment 1376, wherein the Hodgkin's lymphoma is lymphocyte-rich or lymphocytic predominance Hodgkin's lymphoma.

1380. The method or combination of embodiment 1376, wherein the Hodgkin's lymphoma is lymphocyte depleted Hodgkin's lymphoma.

1381. The method or combination of embodiment 1374, wherein the lymphoma is non-Hodgkin's lymphoma.

1382. The method or combination of embodiment 1381, wherein the non-Hodgkin's lymphoma is a B cell lymphoma or a T cell lymphoma.

1383. The method or combination of embodiment 1382, wherein the non-Hodgkin's lymphoma is a B cell lymphoma.

1384. The method or combination of embodiment 1382, wherein the non-Hodgkin's lymphoma is a T cell lymphoma 1385. The method or combination of embodiment 1381, wherein the non-Hodgkin's lymphoma is diffuse large B-cell lymphoma (DLBCL), follicular lymphoma, chronic lymphocytic leukemia (CLL)/small lymphocytic lymphoma (SLL), mantle cell lymphoma (MCL), marginal zone lymphoma, Burkitt lymphoma, lymphoplasmacytic lymphoma (Waldenstrom macroglobulinemia), primary central nervous system (CNS) lymphoma, primary mediastinal large B-cell lymphoma, mediastinal grey-zone lymphoma (MGZL), splenic marginal zone B-cell lymphoma, extranodal marginal zone B-cell lymphoma of MALT, nodal marginal zone B-cell lymphoma, primary effusion lymphoma, anaplastic large cell lymphoma (ALCL), adult T-cell lymphoma, angiocentric lymphoma, angioimmunoblastic T-cell lymphoma, cutaneous T-cell lymphoma, extranodal natural killer/T-cell lymphoma, enteropathy type intestinal T-cell lymphoma, precursor T-lymphoblastic lymphoma, or unspecified peripheral T-cell lymphoma.

1386. The method or combination of embodiment 1385, wherein the non-Hodgkin's lymphoma is diffuse large B-cell lymphoma (DLBCL).

1387. The method or combination of embodiment 1385, wherein the non-Hodgkin's lymphoma is follicular lymphoma.

1388. The method or combination of embodiment 1385, wherein the non-Hodgkin's lymphoma is chronic lymphocytic leukemia (CLL)/small lymphocytic lymphoma (SLL).

1389. The method or combination of embodiment 1385, wherein the non-Hodgkin's lymphoma is mantle cell lymphoma (MCL), marginal zone lymphoma.

1390. The method or combination of embodiment 1385, wherein the non-Hodgkin's lymphoma is Burkitt lymphoma.

1391. The method or combination of embodiment 1385, wherein the non-Hodgkin's lymphoma is lymphoplasmacytic lymphoma (Waldenstrom macroglobulinemia).

1392. The method or combination of embodiment 1385, wherein the non-Hodgkin's lymphoma is primary central nervous system (CNS) lymphoma.

1393. The method or combination of embodiment 1385, wherein the non-Hodgkin's lymphoma is primary mediastinal large B-cell lymphoma.

1394. The method or combination of embodiment 1385, wherein the non-Hodgkin's lymphoma is mediastinal grey-zone lymphoma (MGZL).

1395. The method or combination of embodiment 1385, wherein the non-Hodgkin's lymphoma is splenic marginal zone B-cell lymphoma.

1396. The method or combination of embodiment 1385, wherein the non-Hodgkin's lymphoma is extranodal marginal zone B-cell lymphoma of MALT.

1397. The method or combination of embodiment 1385, wherein the non-Hodgkin's lymphoma is nodal marginal zone B-cell lymphoma.

1398. The method or combination of embodiment 1385, wherein the non-Hodgkin's lymphoma is primary effusion lymphoma, anaplastic large cell lymphoma (ALCL).

1399. The method or combination of embodiment 1385, wherein the non-Hodgkin's lymphoma is adult T-cell lymphoma.

1400. The method or combination of embodiment 1385, wherein the non-Hodgkin's lymphoma is angiocentric lymphoma.

1401. The method or combination of embodiment 1385, wherein the non-Hodgkin's lymphoma is angioimmunoblastic T-cell lymphoma.

1402. The method or combination of embodiment 1385, wherein the non-Hodgkin's lymphoma is cutaneous T-cell lymphoma.

1403. The method or combination of embodiment 1385, wherein the non-Hodgkin's lymphoma is extranodal natural killer/T-cell lymphoma.

1404. The method or combination of embodiment 1385, wherein the non-Hodgkin's lymphoma is enteropathy type intestinal T-cell lymphoma.

1405. The method or combination of embodiment 1385, wherein the non-Hodgkin's lymphoma is precursor T-lymphoblastic lymphoma.

1406. The method or combination of embodiment 1385, wherein the non-Hodgkin's lymphoma is unspecified peripheral T-cell lymphoma.

1407. The method or combination of embodiment 1373, wherein the proliferative disease is a leukemia.

1408. The method or combination of embodiment 1407, wherein the leukemia is B-cell acute lymphoid leukemia (BALL), T-cell acute lymphoid leukemia (TALL), acute lymphoid leukemia (ALL), acute myeloid leukemia (AML), chronic myelogenous leukemia (CML), chronic lymphocytic leukemia (CLL), B-cell chronic lymphocytic leukemia (B-CLL), B-cell prolymphocytic leukemia (B-PLL), hairy cell leukemia, precursor B-lymphoblastic leukemia (PB-LBL), large granular lymphocyte leukemia, precursor T-lymphoblastic leukemia (T-LBL), or T-cell chronic lymphocytic leukemia/prolymphocytic leukemia (T-CLL/PLL).

1409. The method or combination of embodiment 1408, wherein the leukemia is B-cell acute lymphoid leukemia (BALL).

1410. The method or combination of embodiment 1408, wherein the leukemia is T-cell acute lymphoid leukemia (TALL).

1411. The method or combination of embodiment 1408, wherein the leukemia is acute lymphoid leukemia (ALL).

1412. The method or combination of embodiment 1408, wherein the leukemia is acute myeloid leukemia (AML).

1413. The method or combination of embodiment 1408, wherein the leukemia is chronic myelogenous leukemia (CML).

1414. The method or combination of embodiment 1408, wherein the leukemia is chronic lymphocytic leukemia (CLL).

1415. The method or combination of embodiment 1408, wherein the leukemia is B-cell chronic lymphocytic leukemia (B-CLL).

1416. The method or combination of embodiment 1408, wherein the leukemia is B-cell prolymphocytic leukemia (B-PLL).

1417. The method or combination of embodiment 1408, wherein the leukemia is hairy cell leukemia.

1418. The method or combination of embodiment 1408, wherein the leukemia is precursor B-lymphoblastic leukemia (PB-LBL).

1419. The method or combination of embodiment 1408, wherein the leukemia is large granular lymphocyte leukemia.

1420. The method or combination of embodiment 1408, wherein the leukemia is precursor T-lymphoblastic leukemia (T-LBL).

1421. The method or combination of embodiment 1408, wherein the leukemia is T-cell chronic lymphocytic leukemia/prolymphocytic leukemia (T-CLL/PLL).

1422. The method or combination of embodiment 1373, wherein the proliferative disease is multiple myeloma.

1423. The method or combination of embodiment 1373, wherein the proliferative disease is a chronic myeloproliferative neoplasm.

1424. The method or combination of embodiment 1373, wherein the proliferative disease is a macroglobulinemia.

1425. The method or combination of embodiment 1373, wherein the proliferative disease is a myelodysplastic syndrome.

1426. The method or combination of embodiment 1373, wherein the proliferative disease is a myelodysplastic/myeloproliferative neoplasm.

1427. The method or combination of embodiment 1373, wherein the proliferative disease is a plasmacytic dendritic cell neoplasm

1428. The method or combination of embodiment 1370 or 1371, wherein the proliferative disease is adrenocortical carcinoma, anal cancer, appendix cancer, bile duct cancer, bladder cancer, bone cancer, brain cancer, breast cancer, bronchial tumor, carcinoma of unknown primary origin, cervical cancer, a chordoma, colon cancer, colorectal cancer, embryonal tumor, endometrial cancer, ependymoma, esophageal cancer, esthesioneuroblastoma, Ewing sarcoma, eye cancer, malignant fibrous histiocytoma, germ cell tumor, gallbladder cancer, gastric cancer, gastrointestinal carcinoid tumor, gastrointestinal stromal tumor, gestational trophoblastic disease, glioma, head and neck cancer, heart cancer, HER2+ cancer, hypopharyngeal cancer, Kaposi sarcoma, kidney cancer, Langerhans cell histiocytosis, laryngeal cancer, lip and oral cavity cancer, liver cancer, lung cancer, mesothelioma, metastatic squamous neck cancer with occult primary, midline tract carcinoma involving NUT gene, mouth cancer, nasal cavity cancer, nasopharyngeal cancer, neuroblastoma, oropharyngeal cancer, ovarian cancer, pancreatic cancer, para-nasal sinus cancer, paraganglioma, parathyroid cancer, penile cancer, pharyngeal cancer, pituitary cancer, pleuropulmonary blastoma, prostate cancer, rectal cancer, renal cell cancer, renal pelvis and ureter cancer, retinoblastoma, a rhabdoid tumor, salivary gland cancer, skin cancer, small intestine cancer, soft tissue sarcoma, spinal cord tumor, stomach cancer, teratoid tumor, testicular cancer, throat cancer, thymoma, thymic carcinoma, thyroid cancer, urethral cancer, uterine cancer, vaginal cancer, vulvar cancer, or Wilms tumor.

1429. The method or combination of embodiment 1428, wherein the proliferative disease is adrenocortical carcinoma.

1430. The method or combination of embodiment 1428, wherein the proliferative disease is anal cancer.

1431. The method or combination of embodiment 1428, wherein the proliferative disease is appendix cancer.

1432. The method or combination of embodiment 1428, wherein the proliferative disease is bile duct cancer.

1433. The method or combination of embodiment 1428, wherein the proliferative disease is bladder cancer.

1434. The method or combination of embodiment 1428, wherein the proliferative disease is bone cancer.

1435. The method or combination of embodiment 1428, wherein the proliferative disease is brain cancer.

1436. The method or combination of embodiment 1428, wherein the proliferative disease is breast cancer.

1437. The method or combination of embodiment 1428, wherein the proliferative disease is bronchial tumor.

1438. The method or combination of embodiment 1428, wherein the proliferative disease is carcinoma of unknown primary origin.

1439. The method or combination of embodiment 1428, wherein the proliferative disease is cervical cancer.

1440. The method or combination of embodiment 1428, wherein the proliferative disease is a chordoma.

1441. The method or combination of embodiment 1428, wherein the proliferative disease is colon cancer.

1442. The method or combination of embodiment 1428, wherein the proliferative disease is colorectal cancer.

1443. The method or combination of embodiment 1428, wherein the proliferative disease is embryonal tumor.

1444. The method or combination of embodiment 1428, wherein the proliferative disease is endometrial cancer.

1445. The method or combination of embodiment 1428, wherein the proliferative disease is ependymoma.

1446. The method or combination of embodiment 1428, wherein the proliferative disease is esophageal cancer.

1447. The method or combination of embodiment 1428, wherein the proliferative disease is esthesioneuroblastoma.

1448. The method or combination of embodiment 1428, wherein the proliferative disease is Ewing sarcoma.

1449. The method or combination of embodiment 1428, wherein the proliferative disease is eye cancer.

1450. The method or combination of embodiment 1428, wherein the proliferative disease is malignant fibrous histiocytoma.

1451. The method or combination of embodiment 1428, wherein the proliferative disease is germ cell tumor.

1452. The method or combination of embodiment 1428, wherein the proliferative disease is gallbladder cancer.

1453. The method or combination of embodiment 1428, wherein the proliferative disease is gastric cancer.

1454. The method or combination of embodiment 1428, wherein the proliferative disease is gastrointestinal carcinoid tumor.

1455. The method or combination of embodiment 1428, wherein the proliferative disease is gastrointestinal stromal tumor.

1456. The method or combination of embodiment 1428, wherein the proliferative disease is gestational trophoblastic disease.

1457. The method or combination of embodiment 1428, wherein the proliferative disease is glioma.

1458. The method or combination of embodiment 1428, wherein the proliferative disease is head and neck cancer.

1459. The method or combination of embodiment 1428, wherein the proliferative disease is heart cancer.

1460. The method or combination of embodiment 1428, wherein the proliferative disease is HER2+ cancer.

1461. The method or combination of embodiment 1428, wherein the proliferative disease is hypopharyngeal cancer.

1462. The method or combination of embodiment 1428, wherein the proliferative disease is Kaposi sarcoma.

1463. The method or combination of embodiment 1428, wherein the proliferative disease is kidney cancer.

1464. The method or combination of embodiment 1428, wherein the proliferative disease is Langerhans cell histiocytosis.

1465. The method or combination of embodiment 1428, wherein the proliferative disease is laryngeal cancer.

1466. The method or combination of embodiment 1428, wherein the proliferative disease is lip and oral cavity cancer.

1467. The method or combination of embodiment 1428, wherein the proliferative disease is liver cancer.

1468. The method or combination of embodiment 1428, wherein the proliferative disease is lung cancer.

1469. The method or combination of embodiment 1428, wherein the proliferative disease is mesothelioma.

1470. The method or combination of embodiment 1428, wherein the proliferative disease is metastatic squamous neck cancer with occult primary.

1471. The method or combination of embodiment 1428, wherein the proliferative disease is midline tract carcinoma involving NUT gene.

1472. The method or combination of embodiment 1428, wherein the proliferative disease is mouth cancer.

1473. The method or combination of embodiment 1428, wherein the proliferative disease is nasal cavity cancer.

1474. The method or combination of embodiment 1428, wherein the proliferative disease is nasopharyngeal cancer.

1475. The method or combination of embodiment 1428, wherein the proliferative disease is neuroblastoma.

1476. The method or combination of embodiment 1428, wherein the proliferative disease is oropharyngeal cancer.

1477. The method or combination of embodiment 1428, wherein the proliferative disease is ovarian cancer.

1478. The method or combination of embodiment 1428, wherein the proliferative disease is pancreatic cancer.

1479. The method or combination of embodiment 1428, wherein the proliferative disease is para-nasal sinus cancer.

1480. The method or combination of embodiment 1428, wherein the proliferative disease is paraganglioma.

1481. The method or combination of embodiment 1428, wherein the proliferative disease is parathyroid cancer.

1482. The method or combination of embodiment 1428, wherein the proliferative disease is penile cancer.

1483. The method or combination of embodiment 1428, wherein the proliferative disease is pharyngeal cancer.

1484. The method or combination of embodiment 1428, wherein the proliferative disease is pituitary cancer.

1485. The method or combination of embodiment 1428, wherein the proliferative disease is pleuropulmonary blastoma.

1486. The method or combination of embodiment 1428, wherein the proliferative disease is prostate cancer.

1487. The method or combination of embodiment 1428, wherein the proliferative disease is rectal cancer.

1488. The method or combination of embodiment 1428, wherein the proliferative disease is renal cell cancer.

1489. The method or combination of embodiment 1428, wherein the proliferative disease is renal pelvis and ureter cancer.

1490. The method or combination of embodiment 1428, wherein the proliferative disease is retinoblastoma.

1491. The method or combination of embodiment 1428, wherein the proliferative disease is a rhabdoid tumor.

1492. The method or combination of embodiment 1428, wherein the proliferative disease is salivary gland cancer.

1493. The method or combination of embodiment 1428, wherein the proliferative disease is skin cancer.

1494. The method or combination of embodiment 1428, wherein the proliferative disease is small intestine cancer.

1495. The method or combination of embodiment 1428, wherein the proliferative disease is soft tissue sarcoma.

1496. The method or combination of embodiment 1428, wherein the proliferative disease is spinal cord tumor.

1497. The method or combination of embodiment 1428, wherein the proliferative disease is stomach cancer.

1498. The method or combination of embodiment 1428, wherein the proliferative disease is teratoid tumor.

1499. The method or combination of embodiment 1428, wherein the proliferative disease is testicular cancer.

1500. The method or combination of embodiment 1428, wherein the proliferative disease is throat cancer.

1501. The method or combination of embodiment 1428, wherein the proliferative disease is thymoma.

1502. The method or combination of embodiment 1428, wherein the proliferative disease is thymic carcinoma.

1503. The method or combination of embodiment 1428, wherein the proliferative disease is thyroid cancer.

1504. The method or combination of embodiment 1428, wherein the proliferative disease is urethral cancer.

1505. The method or combination of embodiment 1428, wherein the proliferative disease is uterine cancer.

1506. The method or combination of embodiment 1428, wherein the proliferative disease is vaginal cancer.

1507. The method or combination of embodiment 1428, wherein the proliferative disease is vulvar cancer.

1508. The method or combination of embodiment 1428, wherein the proliferative disease is Wilms tumor.

1509. The method or combination of any one of embodiments 1 to 1369, wherein the subject has an autoimmune disorder.

1510. The method or combination of embodiment 1509, wherein the autoimmune disorder is systemic lupus erythematosus (SLE), Sjögren's syndrome, scleroderma, rheumatoid arthritis (RA), juvenile idiopathic arthritis, graft versus host disease, dermatomyositis, type I diabetes mellitus, Hashimoto's thyroiditis, Graves's disease, Addison's disease, celiac disease, Crohn's Disease, pernicious anaemia, pemphigus vulgaris, vitiligo, autoimmune haemolytic anaemia, idiopathic thrombocytopenic purpura, giant cell arteritis, myasthenia gravis, multiple sclerosis (MS) (e.g., relapsing-remitting MS (RRMS)), glomerulonephritis, Goodpasture's syndrome, bullous pemphigoid, colitis ulcerosa, Guillain-Barré syndrome, chronic inflammatory demyelinating polyneuropathy, anti-phospholipid syndrome, narcolepsy, sarcoidosis, or Wegener's granulomatosis.

1511. The method or combination of embodiment 1510, wherein the autoimmune disorder is systemic lupus erythematosus (SLE).

1512. The method or combination of embodiment 1510, wherein the autoimmune disorder is Sjögren's syndrome.

1513. The method or combination of embodiment 1510, wherein the autoimmune disorder is scleroderma.

1514. The method or combination of embodiment 1510, wherein the autoimmune disorder is rheumatoid arthritis (RA).

1515. The method or combination of embodiment 1510, wherein the autoimmune disorder is juvenile idiopathic arthritis.

1516. The method or combination of embodiment 1510, wherein the autoimmune disorder is graft versus host disease.

1517. The method or combination of embodiment 1510, wherein the autoimmune disorder is dermatomyositis.

1518. The method or combination of embodiment 1510, wherein the autoimmune disorder is type I diabetes mellitus.

1519. The method or combination of embodiment 1510, wherein the autoimmune disorder is Hashimoto's thyroiditis.

1520. The method or combination of embodiment 1510, wherein the autoimmune disorder is Graves's disease.

1521. The method or combination of embodiment 1510, wherein the autoimmune disorder is Addison's disease.

1522. The method or combination of embodiment 1510, wherein the autoimmune disorder is celiac disease.

1523. The method or combination of embodiment 1510, wherein the autoimmune disorder is Crohn's Disease.

1524. The method or combination of embodiment 1510, wherein the autoimmune disorder is pernicious anaemia.

1525. The method or combination of embodiment 1510, wherein the autoimmune disorder is pemphigus vulgaris.

1526. The method or combination of embodiment 1510, wherein the autoimmune disorder is vitiligo.

1527. The method or combination of embodiment 1510, wherein the autoimmune disorder is autoimmune haemolytic anaemia.

1528. The method or combination of embodiment 1510, wherein the autoimmune disorder is idiopathic thrombocytopenic purpura.

1529. The method or combination of embodiment 1510, wherein the autoimmune disorder is giant cell arteritis.

1530. The method or combination of embodiment 1510, wherein the autoimmune disorder is myasthenia gravis.

1531. The method or combination of embodiment 1510, wherein the autoimmune disorder is multiple sclerosis (MS).

1532. The method or combination of embodiment 1531, wherein the autoimmune disorder is relapsing-remitting MS (RRMS).

1533. The method or combination of embodiment 1510, wherein the autoimmune disorder is glomerulonephritis.

1534. The method or combination of embodiment 1510, wherein the autoimmune disorder is Goodpasture's syndrome.

1535. The method or combination of embodiment 1510, wherein the autoimmune disorder is bullous pemphigoid.

1536. The method or combination of embodiment 1510, wherein the autoimmune disorder is colitis ulcerosa.

1537. The method or combination of embodiment 1510, wherein the autoimmune disorder is Guillain-Barré syndrome.

1538. The method or combination of embodiment 1510, wherein the autoimmune disorder is chronic inflammatory demyelinating polyneuropathy.

1539. The method or combination of embodiment 1510, wherein the autoimmune disorder is anti-phospholipid syndrome.

1540. The method or combination of embodiment 1510, wherein the autoimmune disorder is narcolepsy.

1541. The method or combination of embodiment 1510, wherein the autoimmune disorder is sarcoidosis.

1542. The method or combination of embodiment 1510, wherein the autoimmune disorder is Wegener's granulomatosis.

1543. The method of any one of embodiments 1 to 1542, further comprising administering to the subject one or more additional agents and/or therapies, optionally wherein the one or more additional agents and/or therapies comprises surgery, chemotherapy, antibodies, radiation, peptide vaccines, steroids, cytoxins, proteasome inhibitors, immunomodulatory drugs (e.g., IMiDs), BH3 mimetics, cytokine therapies, stem cell transplant or any combination thereof.

1544. A kit comprising a first MBM as described in any one of embodiments 1 to 1369 and a second MBM as described in any one of embodiments 1 to 1369.

1545. A first MBM as described in any one of embodiments 1 to 1369.

1546. A second MBM as described in any one of embodiments 1 to 1369.

1547. A first MBM for use in combination with a second MBM for treating a subject having a proliferative disease or an autoimmune disorder, wherein the first MBM is a first MBM as described in any one of embodiments 1 to 1369 and the second MBM is a second MBM as described in any one of embodiments 1 to 1369.

1548. A second MBM for use in combination with a first MBM for treating a subject having a proliferative disease or an autoimmune disorder, wherein the first MBM is a first MBM as described in any one of embodiments 1 to 1369 and the second MBM is a second MBM as described in any one of embodiments 1 to 1369.

1549. The first MBM for use according to embodiment 1547 or the second MBM for use according to embodiment 1548, wherein the proliferative disease or autoimmune disorder is a proliferative disease or autoimmune disorder described in any one of embodiments 1370 to 1542.

1550. A pharmaceutical composition comprising the first MBM of embodiment 1545 and/or the second MBM of embodiment 1546, and an excipient.

1551. A nucleic acid or plurality of nucleic acids encoding the first MBM of embodiment 1545 or the second MBM of embodiment 1546.

1552. The nucleic acid or plurality of nucleic acids of embodiment 1551 which is/are DNA.

1553. The nucleic acid or plurality of nucleic acids of embodiment 1551 which is/are mRNA.

1554. A cell engineered to express the first MBM of embodiment 1545 or the second MBM of embodiment 1546.

1555. A cell transfected with one or more expression vectors comprising one or more nucleic acid sequences encoding the first MBM of embodiment 1545 or the second MBM of embodiment 1546 under the control of one or more promoters.

1556. The cell of embodiment 1554 or embodiment 1555, wherein expression of the MBM is under the control of an inducible promoter.

1557. The cell of any one of embodiments 1554 to 1556, wherein the MBM is produced in secretable form.

1558. A method of producing a MBM, comprising:
(a) culturing the cell of any one of embodiments 1554 to 1557 in conditions under which the MBM is expressed; and
(b) recovering the MBM from the cell culture.

1559. Use of the first MBM of embodiment 1545 in the manufacture of a medicament to treat a proliferative disease or an autoimmune disorder, wherein the medicament is for administration in combination with the second MBM of embodiment 1546.

1560. Use of the second MBM of embodiment 1546 in the manufacture of a medicament to treat a proliferative disease or an autoimmune disorder, wherein the medicament is for administration in combination with the first MBM of embodiment 1545.

1561. The use of embodiment 1559 or 1560, wherein the proliferative disease or autoimmune disorder is a proliferative disease or autoimmune disorder described in any one of embodiments 1370 to 1542.

### 10. INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there are any inconsistencies between the teachings of one or more of the references incorporated herein and the present disclosure, the teachings of the present specification are intended.

What is claimed is:

1. A method of treating a subject having a proliferative disease or an autoimmune disorder, comprising administering to the subject:
(a) a first multispecific binding molecule (“a first MBM”) comprising (i) an antigen-binding module 1 (ABM1) that binds specifically to human CD2 and (ii) an antigen-binding module 2 (ABM2) that binds specifically to a human tumor-associated antigen and/or an antigen-binding module 3 (ABM3) that binds specifically to a human tumor microenvironment antigen; and
(b) a second multispecific binding molecule (“a second MBM”) comprising (i) an antigen-binding module 4 (ABM4) that binds specifically to a component of a human T-cell receptor (TCR) complex or a secondary T-cell signaling molecule and (ii) an antigen-binding module 5 (ABM5) that binds specifically to a human tumor-associated antigen and/or an antigen-binding module 6 (ABM6) that binds specifically to a human tumor microenvironment antigen.

2. A combination for treating a subject having a proliferative disease or an autoimmune disorder, the combination comprising:
(a) a first multispecific binding molecule (“a first MBM”) comprising (i) an antigen-binding module 1 (ABM1) that binds specifically to human CD2 and (ii) an antigen-binding module 2 (ABM2) that binds specifically to a human tumor-associated antigen and/or an antigen-binding module 3 (ABM3) that binds specifically to a human tumor microenvironment antigen; and
(b) a second multispecific binding molecule (“a second MBM”) comprising (i) an antigen-binding module 4 (ABM4) that binds specifically to a component of a human T-cell receptor (TCR) complex or a secondary T-cell signaling molecule and (ii) an antigen-binding module 5 (ABM5) that binds specifically to a human tumor-associated antigen and/or an antigen-binding module 6 (ABM6) that binds specifically to a human tumor microenvironment antigen.

3. The method or combination of claim 1 or claim 2, wherein ABM1 comprises a receptor binding domain of a CD2 ligand.

4. The method or combination of any one of claims 1 to 3, wherein ABM1 is a CD58 moiety.

5. The method or combination of any one of claims 1 to 4, wherein ABM4 binds specifically to a component of a TCR complex.

6. The method or combination of claim 5, wherein the component of the TCR complex is CD3.

7. The method or combination of any one of claims 1 to 6, wherein ABM4 binds specifically to a secondary T-cell signaling molecule.

8. The method or combination of claim 7, wherein the secondary T-cell signaling molecule is a receptor.

9. The method or combination of claim 7, wherein the secondary T-cell signaling molecule is a ligand.

10. The method or combination of claim 7, wherein the secondary T-cell signaling molecule is CD27, CD28, CD30, CD40L, CD150, CD160, CD226, CD244, BTLA, BTN3A1, B7-1, CTLA4, DR3, GITR, HVEM, ICOS, LAG3, LAIR1, LIGHT, OX40, PD1, PDL1, PDL2, TIGIT, TIM1, TIM2, TIM3, VISTA, CD70, or 4-1BB.

11. The method or combination of any one of claims 1 to 10, wherein the first MBM comprises an ABM2 and the second MBM comprises an ABM5 that bind specifically to the same TAA.

12. The method or combination of claim 11, wherein the TAA is CD19, CD20, CD22, CD123, BCMA, CD33, CLL-1, CD138, CS1, CD38, CD133, FLT3, CD52, TNFRSF13C, TNFRSF13B, CXCR4, PD-L1, LY9, CD200, FCGR2B, CD21, CD23, CD24, CD40L, CD72, CD79a, or CD79b.

13. The method or combination of claim 11, wherein the TAA is wherein the TAA is mesothelin, TSHR, CD171, CS-1, CLL-1, GD3, Tn Ag, FLT3, CD38, CD44v6, B7H3, KIT, IL-13Ra2, IL-11Ra, PSCA, PRSS21, VEGFR2, LewisY, CD24, PDGFR-beta, SSEA-4, MUC1, EGFR, NCAM, CAIX, LMP2, EphA2, fucosyl GM1, sLe, GM3, TGS5, HMWMAA, o-acetyl-GD2, GD2, folate receptor alpha, folate receptor beta, TEM1/CD248, TEM7R, CLDN6, GPRC5D, CXORF61, CD97, CD179a, ALK, polysialic acid, PLAC1, GloboH, NY-BR-1, UPK2, HAVCR1, ADRB3, PANX3, GPR20, LY6K, OR51E2, TAARP, WT1, ETV6-AML, sperm protein 17, XAGE1, Tie 2, MAD-CT-1, MAD-CT-2, Fos-related antigen 1, p53 mutant, hTERT, sarcoma translocation breakpoints, ML-IAP, ERG (TMPRSS2 ETS fusion gene), NA17, PAX3, Androgen receptor, Cyclin B1, MYCN, RhoC, CYP1B1, BORIS, SART3, PAX5, OY-TES1, LCK, AKAP-4, SSX2, CD79a, CD79b, CD72, LAIR1, FCAR, LILRA2, CD300LF, CLEC12A, BST2, EMR2, LY75, GPC3, FCRL5, IGLL1, CD19, CD20, CD30, ERBB2, ROR1, TAAG72, CD22, CD33, GD2, BCMA, gp100Tn, FAP, tyrosinase, EPCAM, CEA, Igf-I receptor, EphB2, Cadherin17, CD32b, EGFRvIII, GPNMB, GPR64, HER3, LRP6, LYPD8, NKG2D, SLC34A2, SLC39A6, SLITRK6, TACSTD2, CD123, CD33, CD138, CS1, CD133, CD52, TNFRSF13C, TNFRSF13B, CXCR4, PD-L1, LY9, CD200, FCGR2B, CD21, CD23, or CD40L.

14. The method or combination of any one of claims 11 to 13, wherein ABM2 and ABM5 bind specifically to different epitopes on the same TAA.

15. The method or combination of claim 14, wherein the different epitopes do not overlap.

16. The method or combination of any one of claims 11 to 15, wherein the first MBM and second MBM are capable of specifically binding the TAA simultaneously.

17. The method or combination of any one of claims 11 to 16, wherein binding of the first MBM to the TAA reduces binding of the second MBM to the TAA by less than 50% in a competition assay.

18. The method or combination of any one of claims 11 to 16, wherein binding of the first MBM to the TAA reduces binding of the second MBM to the TAA by less than 40% in a competition assay.

19. The method or combination of any one of claims 11 to 16, wherein binding of the first MBM to the TAA reduces binding of the second MBM to the TAA by less than 30% in a competition assay.

20. The method or combination of any one of claims 11 to 16, wherein binding of the first MBM to the TAA reduces binding of the second MBM to the TAA by less than 20% in a competition assay.

21. The method or combination of any one of claims 11 to 16, wherein binding of the first MBM to the TAA reduces binding of the second MBM to the TAA by less than 10% in a competition assay.

22. The method or combination of any one of claims 17 to 21, wherein the competition assay is an ELISA assay, a Biacore assay, a FACS assay.

23. The method or combination of any one of claims 1 to 10, wherein the first MBM comprises an ABM2 and the second MBM comprises an ABM5 that bind specifically to different TAAs.

24. The method or combination of claim 23, wherein the different TAAs are selected from CD19, CD20, CD22, CD123, BCMA, CD33, CLL-1, CD138, CS1, CD38, CD133, FLT3, CD52, TNFRSF13C, TNFRSF13B, CXCR4, PD-L1, LY9, CD200, FCGR2B, CD21, CD23, CD24, CD40L, CD72, CD79a, and CD79b.

25. The method or combination of claim 23, wherein the different TAAs are selected from mesothelin, TSHR, CD171, CS-1, CLL-1, GD3, Tn Ag, FLT3, CD38, CD44v6, B7H3, KIT, IL-13Ra2, IL-11Ra, PSCA, PRSS21, VEGFR2, LewisY, CD24, PDGFR-beta, SSEA-4, MUC1, EGFR, NCAM, CAIX, LMP2, EphA2, fucosyl GM1, sLe, GM3, TGS5, HMWMAA, o-acetyl-GD2, GD2, folate receptor alpha, folate receptor beta, TEM1/CD248, TEM7R, CLDN6, GPRC5D, CXORF61, CD97, CD179a, ALK, polysialic acid, PLAC1, GloboH, NY-BR-1, UPK2, HAVCR1, ADRB3, PANX3, GPR20, LY6K, OR51E2, TAARP, WT1, ETV6-AML, sperm protein 17, XAGE1, Tie 2, MAD-CT-1, MAD-CT-2, Fos-related antigen 1, p53 mutant, hTERT, sarcoma translocation breakpoints, ML-IAP, ERG (TMPRSS2 ETS fusion gene), NA17, PAX3, Androgen receptor, Cyclin B1, MYCN, RhoC, CYP1B1, BORIS, SART3, PAX5, OY-TES1, LCK, AKAP-4, SSX2, CD79a, CD79b, CD72, LAIR1, FCAR, LILRA2, CD300LF, CLEC12A, BST2, EMR2, LY75, GPC3, FCRL5, IGLL1, CD19, CD20, CD30, ERBB2, ROR1, TAAG72, CD22, CD33, GD2, BCMA, gp100Tn, FAP, tyrosinase, EPCAM, CEA, Igf-I receptor, EphB2, Cadherin17, CD32b, EGFRvIII, GPNMB, GPR64, HER3, LRP6, LYPD8, NKG2D, SLC34A2, SLC39A6, SLITRK6, TACSTD2, CD123, CD33, CD138, CS1, CD133, CD52, TNFRSF13C, TNFRSF13B, CXCR4, PD-L1, LY9, CD200, FCGR2B, CD21, CD23, and CD40L.

26. The method or combination of any one of claims 23 to 25, wherein the different TAAs are expressed on the same cell.

27. The method or combination of any one of claims 23 to 25, wherein the different TAAs are expressed on different cells.

28. The method or combination of any one of claims 1 to 27, wherein the first MBM comprises an ABM3 and the second MBM comprises an ABM6 that bind specifically to the same TMEA.

29. The method or combination of claim 28, wherein the TMEA is APRIL, FAP, BAFF, IL-1R, VEGF-A, VEGFR, CSF1R, ανβ3, or α5β1.

30. The method or combination of any one of claims 1 to 27, wherein the first MBM comprises an ABM3 and the second MBM comprises an ABM6 that bind specifically to different TMEAs.

31. The method or combination of claim 30, wherein the different TMEAs are selected from APRIL, FAP, BAFF, IL-1R, VEGF-A, VEGFR, CSF1R, ανβ3, and α5β1.

32. The method or combination of any one of claims 1 to 31, wherein the first MBM and the second MBM in combination show an additive amount of T cell mediated apoptosis in an in vitro re-directed T cell cytotoxicity assay as compared to the first MBM alone and the second MBM alone.

33. The method or combination of any one of claims 1 to 32, wherein the first MBM and the second MBM in combination show an additive amount of cytokine release in an in vitro cytokine release assay as compared to the first MBM alone and the second MBM alone.

34. The method or combination of any one of claims 1 to 33, wherein the first MBM and the second MBM in combination show an additive amount of T cell proliferation in an in vitro T cell proliferation assay as compared to the first MBM alone and the second MBM alone.

35. The method or combination of any one of claims 1 to 34, wherein the first MBM and the second MBM in combination show an increased amount of T cell activation in an in vitro T cell activation assay as compared to the first MBM alone and the second MBM alone

36. The method or combination of any one of claims 1 to 35, wherein when the first MBM does not comprise an ABM2 or does not comprise an ABM3, the first MBM is a bispecific binding molecule (BBM).

37. The method or combination of any one of claims 1 to 35, wherein the first MBM is a trispecific binding molecule (TBM).

38. The method or combination of any one of claims 1 to 37, wherein when the second MBM does not comprise an ABM5 or does not comprise an ABM6, the second MBM is a bispecific binding molecule (BBM).

39. The method or combination of any one of claims 1 to 37, wherein the second MBM is a trispecific binding molecule (TBM).

40. The method or combination of any one of claims 1 to 39, wherein the TAA to which ABM2 specifically binds, when present, is upregulated in the proliferative disease or autoimmune disorder.

41. The method or combination of any one of claims 1 to 40, wherein the TAA to which ABM5 specifically binds, when present, is upregulated in the proliferative disease or autoimmune disorder.

42. The method or combination of any one of claims 1 to 41, wherein the subject has a proliferative disease.

43. The method or combination of claim 42, wherein the proliferative disease is a cancer or a precancerous condition.

44. The method or combination of claim 42 or 43, wherein the proliferative disease is a hematologic proliferative disease.

45. The method or combination of 44, wherein the proliferative disease is a lymphoma, a leukemia, multiple myeloma, a chronic myeloproliferative neoplasm, a macroglobulinemia, a myelodysplastic syndrome, a myelodysplastic/myeloproliferative neoplasm, or a plasmacytic dendritic cell neoplasm.

46. The method or combination of claim 45, wherein the proliferative disease is a lymphoma.

47. The method or combination of claim 46, wherein the lymphoma is Hodgkin's lymphoma.

48. The method or combination of claim 47, wherein the Hodgkin's lymphoma is nodular sclerosing Hodgkin's lymphoma, mixed-cellularity subtype Hodgkin's lymphoma, lymphocyte-rich or lymphocytic predominance Hodgkin's lymphoma, or lymphocyte depleted Hodgkin's lymphoma.

49. The method or combination of claim 46, wherein the lymphoma is non-Hodgkin's lymphoma.

50. The method or combination of claim 49, wherein the non-Hodgkin's lymphoma is a B cell lymphoma or a T cell lymphoma.

51. The method or combination of claim 49, wherein the non-Hodgkin's lymphoma is diffuse large B-cell lymphoma (DLBCL), follicular lymphoma, chronic lymphocytic leukemia (CLL)/small lymphocytic lymphoma (SLL), mantle cell lymphoma (MCL), marginal zone lymphoma, Burkitt lymphoma, lymphoplasmacytic lymphoma (Waldenstrom macroglobulinemia), primary central nervous system (CNS) lymphoma, primary mediastinal large B-cell lymphoma, mediastinal grey-zone lymphoma (MGZL), splenic marginal zone B-cell lymphoma, extranodal marginal zone B-cell lymphoma of MALT, nodal marginal zone B-cell lymphoma, primary effusion lymphoma, anaplastic large cell lymphoma (ALCL), adult T-cell lymphoma, angiocentric lymphoma, angioimmunoblastic T-cell lymphoma, cutaneous T-cell lymphoma, extranodal natural killer/T-cell lymphoma, enteropathy type intestinal T-cell lymphoma, precursor T-lymphoblastic lymphoma, or unspecified peripheral T-cell lymphoma.

52. The method or combination of claim 45, wherein the proliferative disease is a leukemia.

53. The method or combination of claim 52, wherein the leukemia is B-cell acute lymphoid leukemia (BALL), T-cell acute lymphoid leukemia (TALL), acute lymphoid leukemia (ALL), acute myeloid leukemia (AML), chronic myelogenous leukemia (CML), chronic lymphocytic leukemia (CLL), B-cell chronic lymphocytic leukemia (B-CLL), B-cell prolymphocytic leukemia (B-PLL), hairy cell leukemia, precursor B-lymphoblastic leukemia (PB-LBL), large granular lymphocyte leukemia, precursor T-lymphoblastic leukemia (T-LBL), or T-cell chronic lymphocytic leukemia/prolymphocytic leukemia (T-CLL/PLL).

54. The method or combination of claim 45, wherein the proliferative disease is multiple myeloma.

55. The method or combination of claim 42 or 43, wherein the proliferative disease is adrenocortical carcinoma, anal cancer, appendix cancer, bile duct cancer, bladder cancer, bone cancer, brain cancer, breast cancer, bronchial tumor, carcinoma of unknown primary origin, cervical cancer, a chordoma, colon cancer, colorectal cancer, embryonal tumor, endometrial cancer, ependymoma, esophageal cancer, esthesioneuroblastoma, Ewing sarcoma, eye cancer, malignant fibrous histiocytoma, germ cell tumor, gallbladder cancer, gastric cancer, gastrointestinal carcinoid tumor, gastrointestinal stromal tumor, gestational trophoblastic disease, glioma, head and neck cancer, heart cancer, HER2+ cancer, hypopharyngeal cancer, Kaposi sarcoma, kidney cancer, Langerhans cell histiocytosis, laryngeal cancer, lip and oral cavity cancer, liver cancer, lung cancer, mesothelioma, metastatic squamous neck cancer with occult primary, midline tract carcinoma involving NUT gene, mouth cancer, nasal cavity cancer, nasopharyngeal cancer, neuroblastoma, oropharyngeal cancer, ovarian cancer, pancreatic cancer, para-nasal sinus cancer, paraganglioma, parathyroid cancer, penile cancer, pharyngeal cancer, pituitary cancer, pleuropulmonary blastoma, prostate cancer, rectal cancer, renal cell cancer, renal pelvis and ureter cancer, retinoblastoma, a rhabdoid tumor, salivary gland cancer, skin cancer, small intestine cancer, soft tissue sarcoma, spinal cord tumor, stomach cancer, teratoid tumor, testicular cancer, throat cancer, thymoma, thymic carcinoma, thyroid cancer, urethral cancer, uterine cancer, vaginal cancer, vulvar cancer, or Wilms tumor.

56. The method or combination of any one of claims 1 to 41, wherein the subject has an autoimmune disorder.

57. The method or combination of claim 56, wherein the autoimmune disorder is systemic lupus erythematosus (SLE), Sjögren's syndrome, scleroderma, rheumatoid arthritis (RA), juvenile idiopathic arthritis, graft versus host disease, dermatomyositis, type I diabetes mellitus, Hashimoto's thyroiditis, Graves's disease, Addison's disease, celiac disease, Crohn's Disease, pernicious anaemia, pemphigus vulgaris, vitiligo, autoimmune haemolytic anaemia, idiopathic thrombocytopenic purpura, giant cell arteritis, myasthenia gravis, multiple sclerosis (MS) (e.g., relapsing-remitting MS (RRMS)), glomerulonephritis, Goodpasture's syndrome, bullous pemphigoid, colitis ulcerosa, Guillain-Barré syndrome, chronic inflammatory demyelinating polyneuropathy, anti-phospholipid syndrome, narcolepsy, sarcoidosis, or Wegener's granulomatosis.

58. A kit comprising a first MBM as described in any one of claims 1 to 41 and a second MBM as described in any one of claims 1 to 41.

59. A first MBM as described in any one of claims 1 to 41.

60. A second MBM as described in any one of claims 1 to 41.

61. A first MBM for use in combination with a second MBM for treating a subject having a proliferative disease or an autoimmune disorder, wherein the first MBM is a first MBM as described in any one of claims 1 to 41 and the second MBM is a second MBM as described in any one of claims 1 to 41.

62. A second MBM for use in combination with a first MBM for treating a subject having a proliferative disease or an autoimmune disorder, wherein the first MBM is a first MBM as described in any one of claims 1 to 41 and the second MBM is a second MBM as described in any one of claims 1 to 41.

63. The first MBM for use according to claim 61 or the second MBM for use according to claim 62, wherein the proliferative disease or autoimmune disorder is a proliferative disease or autoimmune disorder described in any one of claims 43 to 57.
